SEQUENCE LISTING (437 sequences)

SEQ ID NO: 1 - DNA, 1536 bp, Homo sapiens
gtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtaaaaaaaaaaaaaaaaaaaaaaaaaaaaaaaaaaa

SEQ ID NO: 2 - DNA, 19 bp, Artificial Sequence
  Synthetic oligonucleotide
tgagagtgattcgcgtggg

SEQ ID NO: 3 - DNA, 21 bp, Artificial Sequence
  Synthetic oligonucleotide
ccagggtacgaggctttcaat

SEQ ID NO: 4 - DNA, 91 bp, Artificial Sequence
  Synthetic oligonucleotide
tgagagtgattcgcgtgggtacccgcaagagccagcttgctcgcatacagacggacagtgtggtggcaacattgaaagcctcgtaccctgg

SEQ ID NO: 5 - DNA, 1331 bp, Homo sapiens
tcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaattt

SEQ ID NO: 6 - DNA, 21 bp, Artificial Sequence
  Synthetic oligonucleotide
tgacactggcaaaacaatgca

SEQ ID NO: 7 - DNA, 21 bp, Artificial Sequence
  Synthetic oligonucleotide
ggtccttttcaccagcaagct

SEQ ID NO: 8 - DNA, 94 bp, Artificial Sequence
  Synthetic oligonucleotide
tgacactggcaaaacaatgcagactttgctttccttggtcaggcagtataatccaaagatggtcaaggtcgcaagcttgctggtgaaaaggacc

SEQ ID NO: 9 - DNA, 1261 bp, Homo sapiens
cttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaa

SEQ ID NO: 10 - DNA, 20 bp, Artificial Sequence
  Synthetic oligonucleotide
tgcaccaccaactgcttagc

SEQ ID NO: 11 - DNA, 19 bp, Artificial Sequence
  Synthetic oligonucleotide
ccatcacgccacagtttcc

SEQ ID NO: 12 - DNA, 116 bp, Artificial Sequence
  Synthetic oligonucleotide
tgcaccaccaactgcttagcacccctggccaaggtcatccatgacaactttggtatcgtggaaggactcatgaccacagtccatgccatcactgccacccagaagactgtggatgg

SEQ ID NO: 13 - DNA, 2650 bp, Homo sapiens
agggacagcccagaggaggcgtggccacgctgccggcggaagtggagccctccgcgagcgcgcgaggccgccggggcaggcggggaaaccggacagtaggggcggggccgggccggcgatggggatgcgggagcactacgcggagctgcacccgtgcccgccggaattggggatgcagagcagcggcagcgggtatggcaggcagccggcgggccggcctccagcgcaggtgcccgagaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtaacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatggatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagccaaaaaaaaaaaaaaaaaaaa

SEQ ID NO: 14 - DNA, 20 bp, Artificial Sequence
  Synthetic oligonucleotide
gaggccgtcaccaagaacat

SEQ ID NO: 15 - DNA, 19 bp, Artificial Sequence
  Synthetic oligonucleotide
ggacagccggtcagagctc

SEQ ID NO: 16 - DNA, 111 bp, Artificial Sequence
  Synthetic oligonucleotide
gaggccgtcaccaagaacattcacgagtcctgcatgagccagataggctggaaccgcatcatcgtggagaagcccttcgggagggacctgcagagctctgaccggctgtcc

SEQ ID NO: 17 - DNA, 541 bp, Homo sapiens
cttttcgatccgccatctgcggtggagccgccaccaaaatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaactgtatgagttaataaaagacatgaactaaca

SEQ ID NO: 18 - DNA, 20 bp, Artificial Sequence
  Synthetic oligonucleotide
ctggcaagcagctggaagat

SEQ ID NO: 19 - DNA, 23 bp, Artificial Sequence
  Synthetic oligonucleotide
tttcttagcaccaccacgaagtc

SEQ ID NO: 20 - DNA, 101 bp, Artificial Sequence
  Synthetic oligonucleotide
ctggcaagcagctggaagatggacgtactttgtctgactacaatattcaaaaggagtctactcttcatcttgtgttgagacttcgtggtggtgctaagaaa

SEQ ID NO: 21 - DNA, 748 bp, Homo sapiens
gcagataatgggaggagccgggcccgagcgagctctttcctttcgctgctgcggccgcagccatgagtatgctcaggcttcagaagaggctcgcctctagtgtcctccgctgtggcaagaagaaggtctggttagaccccaatgagaccaatgaaatcgccaatgccaactcccgtcagcagatccggaagctcatcaaagatgggctgatcatccgcaagcctgtgacggtccattcccgggctcgatgccggaaaaacaccttggcccgccggaagggcaggcacatgggcataggtaagcggaagggtacagccaatgcccgaatgccagagaaggtcacatggatgaggagaatgaggattttgcgccggctgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggcccgcaggtctaagaccaaggaagcacgcaagcgccgtgaagagcgcctccaggccaagaaggaggagatcatcaagactttatccaaggaggaagagaccaagaaataaaacctcccactttgtctgtacatactggcctctgtgattacatagatcagccattaaaataaaacaagccttaatctgcaaaaaaaaaaaaaaaa

SEQ ID NO: 22 - DNA, 23 bp, Artificial Sequence
  Synthetic oligonucleotide
tggcaagaagaaggtctggttag

SEQ ID NO: 23 - DNA, 22 bp, Artificial Sequence
  Synthetic oligonucleotide
tgatcagcccatctttgatgag

SEQ ID NO: 24 - DNA, 101 bp, Artificial Sequence
  Synthetic oligonucleotide
tggcaagaagaaggtctggttagaccccaatgagaccaatgaaatcgccaatgccaactcccgtcagcagatccggaagctcatcaaagatgggctgatca

SEQ ID NO: 25 - DNA, 1867 bp, Homo sapiens
ggttcgctgtggcgggcgcctgggccgccggctgtttaacttcgcttccgctggcccatagtgatctttgcagtgacccagcagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttaaaaaaaaaaaaaaaaaaaaaa

SEQ ID NO: 26 - DNA, 20 bp, Artificial Sequence
  Synthetic oligonucleotide
cggtttgctgcggtaatcat

SEQ ID NO: 27 - DNA, 21 bp, Artificial Sequence
  Synthetic oligonucleotide
tttcttgctgccagtctggac

SEQ ID NO: 28 - DNA, 122 bp, Artificial Sequence
  Synthetic oligonucleotide
cggtttgctgcggtaatcatgaggataagagagccacgaaccacggcactgattttcagttctgggaaaatggtgtgcacaggagccaagagtgaagaacagtccagactggcagcaagaaa

SEQ ID NO: 29 - DNA, 2201 bp, Homo sapiens
cgggatttgggtcgcggttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtggagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgatcagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaa

SEQ ID NO: 30 - DNA, 19 bp, Artificial Sequence
  Synthetic oligonucleotide
atttgggtcgcggttcttg

SEQ ID NO: 31 - DNA, 21 bp, Artificial Sequence
  Synthetic oligonucleotide
tgccttgacattctcgatggt

SEQ ID NO: 32 - DNA, 133 bp, Artificial Sequence
  Synthetic oligonucleotide
atttgggtcgcggttcttgtttgtggatcgctgtgatcgtcacttgacaatgcagatcttcgtgaagactctgactggtaagaccatcaccctcgaggttgagcccagtgacaccatcgagaatgtcaaggca

SEQ ID NO: 33 - DNA, 2301 bp, Homo sapiens
  misc_feature (1)..(2): n is a, c, g, or t
  misc_feature (4)..(4): n is a, c, g, or t
  misc_feature (6)..(11): n is a, c, g, or t
  misc_feature (13)..(16): n is a, c, g, or t
  misc_feature (18)..(23): n is a, c, g, or t
nntncnnnnnnannnntnnnnnnagactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcgcagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggacggtctctgcgatatgataccagctattttgtggagtattttgccttggatctcctgatggagaacggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccccacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtcgatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacagacctggtggagaccctggagctgcagaacctgatgctgtgtgcgctgcagaccatctacggagcagaggcgcggaaggagtcacggggcgcgcatgccagggaagactacaaggtgcggattgatgagtacgattactccaagcccatccaggggcaacagaagaagccctttgaggagcactggaggaagcacaccctgtcctttgtggacgttggcactgggaaggtcactctggaatatagacccgtaatcgacaaaactttgaacgaggctgactgtgccaccatcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgag

SEQ ID NO: 34 - DNA, 20 bp, Artificial Sequence
  Synthetic oligonucleotide
tgggaacaagagggcatctg

SEQ ID NO: 35 - DNA, 22 bp, Artificial Sequence
  Synthetic oligonucleotide
ccaccactgcatcaaattcatg

SEQ ID NO: 36 - DNA, 86 bp, Artificial Sequence
  Synthetic oligonucleotide
tgggaacaagagggcatctgctaaagtttcagattccatttctgctcagtatccagtagtggatcatgaatttgatgcagtggtgg

SEQ ID NO: 37 - DNA, 3566 bp, Homo sapiens
gctcaagtgccctgccttgccccacccagcccagcctggccagagccccctggagaaggagctctcttcttgcttggcagctggaccaagggagccagtcttgggcgctggagggcctgtcctgaccatggtccctgcctggctgtggctgctttgtgtctccgtcccccaggctctccccaaggcccagcctgcagagctgtctgtggaagttccagaaaactatggtggaaatttccctttatacctgaccaagttgccgctgccccgtgagggggctgaaggccagatcgtgctgtcaggggactcaggcaaggcaactgagggcccatttgctatggatccagattctggcttcctgctggtgaccagggccctggaccgagaggagcaggcagagtaccagctacaggtcaccctggagatgcaggatggacatgtcttgtggggtccacagcctgtgcttgtgcacgtgaaggatgagaatgaccaggtgccccatttctctcaagccatctacagagctcggctgagccggggtaccaggcctggcatccccttcctcttccttgaggcttcagaccgggatgagccaggcacagccaactcggatcttcgattccacatcctgagccaggctccagcccagccttccccagacatgttccagctggagcctcggctgggggctctggccctcagccccaaggggagcaccagccttgaccacgccctggagaggacctaccagctgttggtacaggtcaaggacatgggtgaccaggcctcaggccaccaggccactgccaccgtggaagtctccatcatagagagcacctgggtgtccctagagcctatccacctggcagagaatctcaaagtcctatacccgcaccacatggcccaggtacactggagtgggggtgatgtgcactatcacctggagagccatcccccgggaccctttgaagtgaatgcagagggaaacctctacgtgaccagagagctggacagagaagcccaggctgagtacctgctccaggtgcgggctcagaattcccatggcgaggactatgcggcccctctggagctgcacgtgctggtgatggatgagaatgacaacgtgcctatctgccctccccgtgaccccacagtcagcatccctgagctcagtccaccaggtactgaagtgactagactgtcagcagaggatgcagatgcccccggctcccccaattcccacgttgtgtatcagctcctgagccctgagcctgaggatggggtagaggggagagccttccaggtggaccccacttcaggcagtgtgacgctgggggtgctcccactccgagcaggccagaacatcctgcttctggtgctggccatggacctggcaggcgcagagggtggcttcagcagcacgtgtgaagtcgaagtcgcagtcacagatatcaatgatcacgcccctgagttcatcacttcccagattgggcctataagcctccctgaggatgtggagcccgggactctggtggccatgctaacagccattgatgctgacctcgagcccgccttccgcctcatggattttgccattgagaggggagacacagaagggacttttggcctggattgggagccagactctgggcatgttagactcagactctgcaagaacctcagttatgaggcagctccaagtcatgaggtggtggtggtggtgcagagtgtggcgaagctggtggggccaggcccaggccctggagccaccgccacggtgactgtgctagtggagagagtgatgccaccccccaagttggaccaggagagctacgaggccagtgtccccatcagtgccccagccggctctttcctgctgaccatccagccctccgaccccatcagccgaaccctcaggttctccctagtcaatgactcagagggctggctctgcattgagaaattctccggggaggtgcacaccgcccagtccctgcagggcgcccagcctggggacacctacacggtgcttgtggaggcccaggatacaggtatggccaggcagtgatgggagcccctgggagggggtacaaacctcagacagacagacagcaggactgagcttcctcccagaggccctcactggcaacccccagccccaagcaggagaggcagatatgaagctgggttcaatgttcctctctgagaatggatctactcctccggtgggcaagtctggcatctgcctcccctctctgatcattctcctagattgacacacaaaatctcccagccctgtcctttagccactggagggcctctcagggtgtcagcttcagggactggtatagggccggggaggtggcaggaggaccaggggctgcccctggggaaggagtgctggagtgaatgggccactggagccaggtgtggaggacactgaacactgctcaggacagagagctggccctggggtggggagccccgggaggcaggtttcaactctgagctgggctgtgatacagggcagacccagcagcagtgagctccgccaaagtcagggatgtccacagtggcatggggatgatgacaggtggagagaagcatggagggagcatggcaggggagtctataaggcgaggctcatgggctgcgggagcagcccagggacctcagagctcaccctccccctggcaacacctggccagctcagcactgcctcctctgcagatgagccgagactgagcgcttctgcacccctggtgatccacttcctaaaggcccctcctgccccagccctgactcttgcccctgtgccctcccaatacctctgcacaccccgccaagaccatggcttgatcgtgagtggacccagcaaggaccccgatctggccagtgggcacggtccctacagcttcacccttggtcccaaccccacggtgcaacgggattggcgcctccagactctcaatggttcccatgcctacctcaccttggccctgcattgggtggagccacgtgaacacataatccccgtggtggtcagccacaatgcccagatgtggcagctcctggttcgagtgatcgtgtgtcgctgcaacgtggaggggcagtgcatgcgcaaggtgggccgcatgaagggcatgcccacgaagctgtcggcagtgggcatccttgtaggcaccctggtagcaataggaatcttcctcatcctcattttcacccactggaccatgtcaaggaagaaggacccggatcaaccagcagacagcgtgcccctgaaggcgactgtctgaatggcccaggcagctctagctgggagcttggcctctggctccatctgagtcccctgggagagagcccagcacccaagatccagcaggggacaggacagagtagaagcccctccatctgccctggggtggaggcaccatcaccatcaccaggcatgtctgcagagcctggacaccaactttatggactgcccatgggagtgctccaaatgtcagggtgtttgcccaataataaagccccagagaactgggctgggccctatgggattggta

SEQ ID NO: 38 - DNA, 3096 bp, Homo sapiens
gctcaagtgccctgccttgccccacccagcccagcctggccagagccccctggagaaggagctctcttcttgcttggcagctggaccaagggagccagtcttgggcgctggagggcctgtcctgaccatggtccctgcctggctgtggctgctttgtgtctccgtcccccaggctctccccaaggcccagcctgcagagctgtctgtggaagttccagaaaactatggtggaaatttccctttatacctgaccaagttgccgctgccccgtgagggggctgaaggccagatcgtgctgtcaggggactcaggcaaggcaactgagggcccatttgctatggatccagattctggcttcctgctggtgaccagggccctggaccgagaggagcaggcagagtaccagctacaggtcaccctggagatgcaggatggacatgtcttgtggggtccacagcctgtgcttgtgcacgtgaaggatgagaatgaccaggtgccccatttctctcaagccatctacagagctcggctgagccggggtaccaggcctggcatccccttcctcttccttgaggcttcagaccgggatgagccaggcacagccaactcggatcttcgattccacatcctgagccaggctccagcccagccttccccagacatgttccagctggagcctcggctgggggctctggccctcagccccaaggggagcaccagccttgaccacgccctggagaggacctaccagctgttggtacaggtcaaggacatgggtgaccaggcctcaggccaccaggccactgccaccgtggaagtctccatcatagagagcacctgggtgtccctagagcctatccacctggcagagaatctcaaagtcctatacccgcaccacatggcccaggtacactggagtgggggtgatgtgcactatcacctggagagccatcccccgggaccctttgaagtgaatgcagagggaaacctctacgtgaccagagagctggacagagaagcccaggctgaggtgatatgaagggggagccagggatgcaggctgggggtgcttggtcagtgtccagttggcttttgggggtgtcacaactacaacagaggctgaggcagtgggaaagagccccaaagagctggagctggcccaccggaaggaaactcagagccaggcaaggctggagctgcccttggtcctgacctgaagccccctactgcccaccgcagtacctgctccaggtgcgggctcagaattcccatggcgaggactatgcggcccctctggagctgcacgtgctggtgatggatgagaatgacaacgtgcctatctgccctccccgtgaccccacagtcagcatccctgagctcagtccaccaggtactgaagtgactagactgtcagcagaggatgcagatgcccccggctcccccaattcccacgttgtgtatcagctcctgagccctgagcctgaggatggggtagaggggagagccttccaggtggaccccacttcaggcagtgtgacgctgggggtgctcccactccgagcaggccagaacatcctgcttctggtgctggccatggacctggcaggcgcagagggtggcttcagcagcacgtgtgaagtcgaagtcgcagtcacagatatcaatgatcacgcccctgagttcatcacttcccagattgggcctataagcctccctgaggatgtggagcccgggactctggtggccatgctaacagccattgatgctgacctcgagcccgccttccgcctcatggattttgccattgagaggggagacacagaagggacttttggcctggattgggagccagactctgggcatgttagactcagactctgcaagaacctcagttatgaggcagctccaagtcatgaggtggtggtggtggtgcagagtgtggcgaagctggtggggccaggcccaggccctggagccaccgccacggtgactgtgctagtggagagagtgatgccaccccccaagttggaccaggagagctacgaggccagtgtccccatcagtgccccagccggctctttcctgctgaccatccagccctccgaccccatcagccgaaccctcaggttctccctagtcaatgactcagagggctggctctgcattgagaaattctccggggaggtgcacaccgcccagtccctgcagggcgcccagcctggggacacctacacggtgcttgtggaggcccaggatacagatgagccgagactgagcgcttctgcacccctggtgatccacttcctaaaggcccctcctgccccagccctgactcttgcccctgtgccctcccaatacctctgcacaccccgccaagaccatggcttgatcgtgagtggacccagcaaggaccccgatctggccagtgggcacggtccctacagcttcacccttggtcccaaccccacggtgcaacgggattggcgcctccagactctcaatggttcccatgcctacctcaccttggccctgcattgggtggagccacgtgaacacataatccccgtggtggtcagccacaatgcccagatgtggcagctcctggttcgagtgatcgtgtgtcgctgcaacgtggaggggcagtgcatgcgcaaggtgggccgcatgaagggcatgcccacgaagctgtcggcagtgggcatccttgtaggcaccctggtagcaataggaatcttcctcatcctcattttcacccactggaccatgtcaaggaagaaggacccggatcaaccagcagacagcgtgcccctgaaggcgactgtctgaatggcccaggcagctctagctgggagcttggcctctggctccatctgagtcccctgggagagagcccagcacccaagatccagcaggggacaggacagagtagaagcccctccatctgccctggggtggaggcaccatcaccatcaccaggcatgtctgcagagcctggacaccaactttatggactgcccatgggagtgctccaaatgtcagggtgtttgcccaataataaagccccagagaactgggctgggccctatgggattggta

SEQ ID NO: 39 - DNA, 2877 bp, Homo sapiens
gctcaagtgccctgccttgccccacccagcccagcctggccagagccccctggagaaggagctctcttcttgcttggcagctggaccaagggagccagtcttgggcgctggagggcctgtcctgaccatggtccctgcctggctgtggctgctttgtgtctccgtcccccaggctctccccaaggcccagcctgcagagctgtctgtggaagttccagaaaactatggtggaaatttccctttatacctgaccaagttgccgctgccccgtgagggggctgaaggccagatcgtgctgtcaggggactcaggcaaggcaactgagggcccatttgctatggatccagattctggcttcctgctggtgaccagggccctggaccgagaggagcaggcagagtaccagctacaggtcaccctggagatgcaggatggacatgtcttgtggggtccacagcctgtgcttgtgcacgtgaaggatgagaatgaccaggtgccccatttctctcaagccatctacagagctcggctgagccggggtaccaggcctggcatccccttcctcttccttgaggcttcagaccgggatgagccaggcacagccaactcggatcttcgattccacatcctgagccaggctccagcccagccttccccagacatgttccagctggagcctcggctgggggctctggccctcagccccaaggccttgaccacgccctggagaggacctaccagctgttggtacaggtcaaggacatgggtgaccaggcctcaggccaccaggccactgccaccgtggaagtctccatcatagagagcacctgggtgtccctagagcctatccacctggcagagaatctcaaagtcctatacccgcaccacatggcccaggtacactggagtgggggtgatgtgcactatcacctggagagccatcccccgggaccctttgaagtgaatgcagagggaaacctctacgtgaccagagagctggacagagaagcccaggctgagtacctgctccaggtgcgggctcagaattcccatggcgaggactatgcggcccctctggagctgcacgtgctggtgatggatgagaatgacaacgtgcctatctgccctccccgtgaccccacagtcagcatccctgagctcagtccaccaggtactgaagtgactagactgtcagcagaggatgcagatgcccccggctcccccaattcccacgttgtgtatcagctcctgagccctgagcctgaggatggggtagaggggagagccttccaggtggaccccacttcaggcagtgtgacgctgggggtgctcccactccgagcaggccagaacatcctgcttctggtgctggccatggacctggcaggcgcagagggtggcttcagcagcacgtgtgaagtcgaagtcgcagtcacagatatcaatgatcacgcccctgagttcatcacttcccagattgggcctataagcctccctgaggatgtggagcccgggactctggtggccatgctaacagccattgatgctgacctcgagcccgccttccgcctcatggattttgccattgagaggggagacacagaagggacttttggcctggattgggagccagactctgggcatgttagactcagactctgcaagaacctcagttatgaggcagctccaagtcatgaggtggtggtggtggtgcagagtgtggcgaagctggtggggccaggcccaggccctggagccaccgccacggtgactgtgctagtggagagagtgatgccaccccccaagttggaccaggagagctacgaggccagtgtccccatcagtgccccagccggctctttcctgctgaccatccagccctccgaccccatcagccgaaccctcaggttctccctagtcaatgactcagagggctggctctgcattgagaaattctccggggaggtgcacaccgcccagtccctgcagggcgcccagcctggggacacctacacggtgcttgtggaggcccaggatacagatgagccgagactgagcgcttctgcacccctggtgatccacttcctaaaggcccctcctgccccagccctgactcttgcccctgtgccctcccaatacctctgcacaccccgccaagaccatggcttgatcgtgagtggacccagcaaggaccccgatctggccagtgggcacggtccctacagcttcacccttggtcccaaccccacggtgcaacgggattggcgcctccagactctcaatggttcccatgcctacctcaccttggccctgcattgggtggagccacgtgaacacataatccccgtggtggtcagccacaatgcccagatgtggcagctcctggttcgagtgatcgtgtgtcgctgcaacgtggaggggcagtgcatgcgcaaggtgggccgcatgaagggcatgcccacgaagctgtcggcagtgggcatccttgtaggcaccctggtagcaataggaatcttcctcatcctcattttcacccactggaccatgtcaaggaagaaggacccggatcaaccagcagacagcgtgcccctgaaggcgactgtctgaatggcccaggcagctctagctgggagcttggcctctggctccatctgagtcccctgggagagagcccagcacccaagatccagcaggggacaggacagagtagaagcccctccatctgccctggggtggaggcaccatcaccatcaccaggcatgtctgcagagcctggacaccaactttatggactgcccatgggagtgctccaaatgtcagggtgtttgcccaataataaagccccagagaactgggctgggccctatgggattggta

SEQ ID NO: 40 - DNA, 2864 bp, Homo sapiens
gctcaagtgccctgccttgccccacccagcccagcctggccagagccccctggagaaggagctctcttcttgcttggcagctggaccaagggagccagtcttgggcgctggagggcctgtcctgaccatggtccctgcctggctgtggctgctttgtgtctccgtcccccaggctctccccaaggcccagcctgcagagctgtctgtggaagttccagaaaactatggtggaaatttccctttatacctgaccaagttgccgctgccccgtgagggggctgaaggccagatcgtgctgtcaggggactcaggcaaggcaactgagggcccatttgctatggatccagattctggcttcctgctggtgaccagggccctggaccgagaggagcaggcagagtaccagctacaggtcaccctggagatgcaggatggacatgtcttgtggggtccacagcctgtgcttgtgcacgtgaaggatgagaatgaccaggtgccccatttctctcaagccatctacagagctcggctgagccggggtaccaggcctggcttcagaccgggatgagccaggcacagccaactcggatcttcgattccacatcctgagccaggctccagcccagccttccccagacatgttccagctggagcctcggctgggggctctggccctcagccccaaggggagcaccagccttgaccacgccctggagaggacctaccagctgttggtacaggtcaaggacatgggtgaccaggcctcaggccaccaggccactgccaccgtggaagtctccatcatagagagcacctgggtgtccctagagcctatccacctggcagagaatctcaaagtcctatacccgcaccacatggcccaggtacactggagtgggggtgatgtgcactatcacctggagagccatcccccgggaccctttgaagtgaatgcagagggaaacctctacgtgaccagagagctggacagagaagcccaggctgagtacctgctccaggtgcgggctcagaattcccatggcgaggactatgcggcccctctggagctgcacgtgctggtgatggatgagaatgacaacgtgcctatctgccctccccgtgaccccacagtcagcatccctgagctcagtccaccaggtactgaagtgactagactgtcagcagaggatgcagatgcccccggctcccccaattcccacgttgtgtatcagctcctgagccctgagcctgaggatggggtagaggggagagccttccaggtggaccccacttcaggcagtgtgacgctgggggtgctcccactccgagcaggccagaacatcctgcttctggtgctggccatggacctggcaggcgcagagggtggcttcagcagcacgtgtgaagtcgaagtcgcagtcacagatatcaatgatcacgcccctgagttcatcacttcccagattgggcctataagcctccctgaggatgtggagcccgggactctggtggccatgctaacagccattgatgctgacctcgagcccgccttccgcctcatggattttgccattgagaggggagacacagaagggacttttggcctggattgggagccagactctgggcatgttagactcagactctgcaagaacctcagttatgaggcagctccaagtcatgaggtggtggtggtggtgcagagtgtggcgaagctggtggggccaggcccaggccctggagccaccgccacggtgactgtgctagtggagagagtgatgccaccccccaagttggaccaggagagctacgaggccagtgtccccatcagtgccccagccggctctttcctgctgaccatccagccctccgaccccatcagccgaaccctcaggttctccctagtcaatgactcagagggctggctctgcattgagaaattctccggggaggtgcacaccgcccagtccctgcagggcgcccagcctggggacacctacacggtgcttgtggaggcccaggatacagatgagccgagactgagcgcttctgcacccctggtgatccacttcctaaaggcccctcctgccccagccctgactcttgcccctgtgccctcccaatacctctgcacaccccgccaagaccatggcttgatcgtgagtggacccagcaaggaccccgatctggccagtgggcacggtccctacagcttcacccttggtcccaaccccacggtgcaacgggattggcgcctccagactctcaatggttcccatgcctacctcaccttggccctgcattgggtggagccacgtgaacacataatccccgtggtggtcagccacaatgcccagatgtggcagctcctggttcgagtgatcgtgtgtcgctgcaacgtggaggggcagtgcatgcgcaaggtgggccgcatgaagggcatgcccacgaagctgtcggcagtgggcatccttgtaggcaccctggtagcaataggaatcttcctcatcctcattttcacccactggaccatgtcaaggaagaaggacccggatcaaccagcagacagcgtgcccctgaaggcgactgtctgaatggcccaggcagctctagctgggagcttggcctctggctccatctgagtcccctgggagagagcccagcacccaagatccagcaggggacaggacagagtagaagcccctccatctgccctggggtggaggcaccatcaccatcaccaggcatgtctgcagagcctggacaccaactttatggactgcccatgggagtgctccaaatgtcagggtgtttgcccaataataaagccccagagaactgggctgggccctatgggattggta

SEQ ID NO: 41 - DNA, 3203 bp, Homo sapiens
gctcaagtgccctgccttgccccacccagcccagcctggccagagccccctggagaaggagctctcttcttgcttggcagctggaccaagggagccagtcttgggcgctggagggcctgtcctgaccatggtccctgcctggctgtggctgctttgtgtctccgtcccccaggctctccccaaggcccagcctgcagagctgtctgtggaagttccagaaaactatggtggaaatttccctttatacctgaccaagttgccgctgccccgtgagggggctgaaggccagatcgtgctgtcaggggactcaggcaaggcaactgagggcccatttgctatggatccagattctggcttcctgctggtgaccagggccctggaccgagaggagcaggcagagtaccagctacaggtcaccctggagatgcaggatggacatgtcttgtggggtccacagcctgtgcttgtgcacgtgaaggatgagaatgaccaggtgccccatttctctcaagccatctacagagctcggctgagccggggtaccaggcctggcatccccttcctcttccttgaggcttcagaccgggatgagccaggcacagccaactcggatcttcgattccacatcctgagccaggctccagcccagccttccccagacatgttccagctggagcctcggctgggggctctggccctcagccccaaggggagcaccagccttgaccacgccctggagaggacctaccagctgttggtacaggtcaaggacatgggtgaccaggcctcaggccaccaggccactgccaccgtggaagtctccatcatagagagcacctgggtgtccctagagcctatccacctggcagagaatctcaaagtcctatacccgcaccacatggcccaggtacactggagtgggggtgatgtgcactatcacctggagagccatcccccgggaccctttgaagtgaatgcagagggaaacctctacgtgaccagagagctggacagagaagcccaggctgagtacctgctccaggtgcgggctcagaattcccatggcgaggactatgcggcccctctggagctgcacgtgctggtgatggatgagaatgacaacgtgcctatctgccctccccgtgaccccacagtcagcatccctgagctcagtccaccaggtactgaagtgactagactgtcagcagaggatgcagatgcccccggctcccccaattcccacgttgtgtatcagctcctgagccctgagcctgaggatggggtagaggggagagccttccaggtggaccccacttcaggcagtgtgacgctgggggtgctcccactccgagcaggccagaacatcctgcttctggtgctggccatggacctggcaggcgcagagggtggcttcagcagcacgtgtgaagtcgaagtcgcagtcacagatatcaatgatcacgcccctgagttcatcacttcccagattgggcctataagcctccctgaggatgtggagcccgggactctggtggccatgctaacagccattgatgctgacctcgagcccgccttccgcctcatggattttgccattgagaggggagacacagaagggacttttggcctggattgggagccagactctgggcatgttagactcagactctgcaagaacctcagttatgaggcagctccaagtcatgaggtggtggtggtggtgcagagtgtggcgaagctggtggggccaggcccaggccctggagccaccgccacggtgactgtgctagtggagagagtgatgccaccccccaagttggaccaggagagctacgaggccagtgtccccatcagtgccccagccggctctttcctgctgaccatccagccctccgaccccatcagccgaaccctcaggttctccctagtcaatgactcagagggctggctctgcattgagaaattctccggggaggtgcacaccgcccagtccctgcagggcgcccagcctggggacacctacacggtgcttgtggaggcccaggatacagatgagccgagactgagcgcttctgcacccctggtgatccacttcctaaaggcccctcctgccccagccctgactcttgcccctgtgccctcccaatacctctgcacaccccgccaagaccatggcttgatcgtgagtggacccagcaaggaccccgatctggccagtgggcacggtccctacagcttcacccttggtcccaaccccacggtgcaacgggattggcgcctccagactctcaatggttcccatgcctacctcaccttggccctgcattgggtggagccacgtgaacacataatccccgtggtggtcagccacaatgcccagatgtggcagctcctggttcgagtgatcgtgtgtcgctgcaacgtggaggggcagtgcatgcgcaaggtgggccgcatgaagggcatgcccacgaagctgtcggcagtgggcatccttgtaggcaccctggtagcaatagcacactgctgtgatgagcctttctcttggaggtccatggagttgggatccaacttactggttccccgatgtccccaagagaaggggactttggaggctgagtatactgggtcagcctcagaggggtaggggccaagccggccgaggagtcaagcttctccaggcaccaccacagagtcatacccagcccagcctcaggatgctgtccaggaaactcatctcttcattaatccaatgccaatcacaggaagtccctcgtgatgaacaagtcccacagccccttctgaggcctgatacatccctcttgggccccacaggaatcttcctcatcctcattttcacccactggaccatgtcaaggaagaaggacccggatcaaccagcagacagcgtgcccctgaaggcgactgtctgaatggcccaggcagctctagctgggagcttggcctctggctccatctgagtcccctgggagagagcccagcacccaagatccagcaggggacaggacagagtagaagcccctccatctgccctggggtggaggcaccatcaccatcaccaggcatgtctgcagagcctggacaccaactttatggactgcccatgggagtgctccaaatgtcagggtgtttgcccaataataaagccccagagaactgggctgggccctatgggattggta

SEQ ID NO: 42 - DNA, 3579 bp, Homo sapiens
gctcaagtgccctgccttgccccacccagcccagcctggccagagccccctggagaaggagctctcttcttgcttggcagctggaccaagggagccagtcttgggcgctggagggcctgtcctgaccatggtccctgcctggctgtggctgctttgtgtctccgtcccccaggctctccccaaggcccagcctgcagagctgtctgtggaagttccagaaaactatggtggaaatttccctttatacctgaccaagttgccgctgccccgtgagggggctgaaggccagatcgtgctgtcaggggactcaggcaaggcaactgagggcccatttgctatggatccagattctggcttcctgctggtgaccagggccctggaccgagaggagcaggcagagtaccagctacaggtcaccctggagatgcaggatggacatgtcttgtggggtccacagcctgtgcttgtgcacgtgaaggatgagaatgaccaggtgccccatttctctcaagccatctacagagctcggctgagccggggtaccaggcctgtttgccctcccaccgtgaaccaggcatccccttcctcttccttgaggcttcagaccgggatgagccaggcacagccaactcggatcttcgattccacatcctgagccaggctccagcccagccttccccagacatgttccagctggagcctcggctgggggctctggccctcagccccaaggccttgaccacgccctggagaggacctaccagctgttggtacaggtcaaggacatgggtgaccaggcctcaggccaccaggccactgccaccgtggaagtctccatcatagagagcacctgggtgtccctagagcctatccacctggcagagaatctcaaagtcctatacccgcaccacatggcccaggtacactggagtgggggtgatgtgcactatcacctggagagccatcccccgggaccctttgaagtgaatgcagagggaaacctctacgtgaccagagagctggacagagaagcccaggctgagtacctgctccaggtgcgggctcagaattcccatggcgaggactatgcggcccctctggagctgcacgtgctggtgatggatgagaatgacaacgtgcctatctgccctccccgtgaccccacagtcagcatccctgagctcagtccaccaggtactgaagtgactagactgtcagcagaggatgcagatgcccccggctcccccaattcccacgttgtgtatcagctcctgagccctgagcctgaggatggggtagaggggagagccttccaggtggaccccacttcaggcagtgtgacgctgggggtgctcccactccgagcaggccagaacatcctgcttctggtgctggccatggacctggcaggcgcagagggtggcttcagcagcacgtgtgaagtcgaagtcgcagtcacagatatcaatgatcacgcccctgagttcatcacttcccagattgggcctataagcctccctgaggatgtggagcccgggactctggtggccatgctaacagccattgatgctgacctcgagcccgccttccgcctcatggattttgccattgagaggggagacacagaagggacttttggcctggattgggagccagactctgggcatgttagactcagactctgcaagaacctcagttatgaggcagctccaagtcatgaggtggtggtggtggtgcagagtgtggcgaagctggtggggccaggcccaggccctggagccaccgccacggtgactgtgctagtggagagagtgatgccaccccccaagttggaccaggagagctacgaggccagtgtccccatcagtgccccagccggctctttcctgctgaccatccagccctccgaccccatcagccgaaccctcaggttctccctagtcaatgactcagagggctggctctgcattgagaaattctccggggaggtgcacaccgcccagtccctgcagggcgcccagcctggggacacctacacggtgcttgtggaggcccaggatacaggtatggccaggcagtgatgggagcccctgggagggggtacaaacctcagacagacagacagcaggactgagcttcctcccagaggccctcactggcaacccccagccccaagcaggagaggcagatatgaagctgggttcaatgttcctctctgagaatggatctactcctccggtgggcaagtctggcatctgcctcccctctctgatcattctcctagattgacacacaaaatctcccagccctgtcctttagccactggagggcctctcagggtgtcagcttcagggactggtatagggccggggaggtggcaggaggaccaggggctgcccctggggaaggagtgctggagtgaatgggccactggagccaggtgtggaggacactgaacactgctcaggacagagagctggccctggggtggggagccccgggaggcaggtttcaactctgagctgggctgtgatacagggcagacccagcagcagtgagctccgccaaagtcagggatgtccacagtggcatggggatgatgacaggtggagagaagcatggagggagcatggcaggggagtctataaggcgaggctcatgggctgcgggagcagcccagggacctcagagctcaccctccccctggcaacacctggccagctcagcactgcctcctctgcagatgagccgagactgagcgcttctgcacccctggtgatccacttcctaaaggcccctcctgccccagccctgactcttgcccctgtgccctcccaatacctctgcacaccccgccaagaccatggcttgatcgtgagtggacccagcaaggaccccgatctggccagtgggcacggtccctacagcttcacccttggtcccaaccccacggtgcaacgggattggcgcctccagactctcaatggttcccatgcctacctcaccttggccctgcattgggtggagccacgtgaacacataatccccgtggtggtcagccacaatgcccagatgtggcagctcctggttcgagtgatcgtgtgtcgctgcaacgtggaggggcagtgcatgcgcaaggtgggccgcatgaagggcatgcccacgaagctgtcggcagtgggcatccttgtaggcaccctggtagcaataggaatcttcctcatcctcattttcacccactggaccatgtcaaggaagaaggacccggatcaaccagcagacagcgtgcccctgaaggcgactgtctgaatggcccaggcagctctagctgggagcttggcctctggctccatctgagtcccctgggagagagcccagcacccaagatccagcaggggacaggacagagtagaagcccctccatctgccctggggtggaggcaccatcaccatcaccaggcatgtctgcagagcctggacaccaactttatggactgcccatgggagtgctccaaatgtcagggtgtttgcccaataataaagccccagagaactgggctgggccctatgggattggta

SEQ ID NO: 43 - DNA, 156 bp, Homo sapiens
ttgccgctgccccgtgagggggctgaaggccagatcgtgctgtcaggggactcaggcaaggcaactgagggcccatttgctatggatccagattctggcttcctgctggtgaccagggccctggaccgagaggagcaggcagagtaccagctacag

SEQ ID NO: 44 - DNA, 136 bp, Homo sapiens
gcttcagaccgggatgagccaggcacagccaactcggatcttcgattccacatcctgagccaggctccagcccagccttccccagacatgttccagctggagcctcggctgggggctctggccctcagccccaagg

SEQ ID NO: 45 - DNA, 123 bp, Homo sapiens
gcctcaggccaccaggccactgccaccgtggaagtctccatcatagagagcacctgggtgtccctagagcctatccacctggcagagaatctcaaagtcctatacccgcaccacatggcccag

SEQ ID NO: 46 - DNA, 123 bp, Homo sapiens
gtacactggagtgggggtgatgtgcactatcacctggagagccatcccccgggaccctttgaagtgaatgcagagggaaacctctacgtgaccagagagctggacagagaagcccaggctgag

SEQ ID NO: 47 - DNA, 209 bp, Homo sapiens
gtgatatgaagggggagccagggatgcaggctgggggtgcttggtcagtgtccagttggcttttgggggtgtcacaactacaacagaggctgaggcagtgggaaagagccccaaagagctggagctggcccaccggaaggaaactcagagccaggcaaggctggagctgcccttggtcctgacctgaagccccctactgcccaccgcag

SEQ ID NO: 48 - DNA, 151 bp, Homo sapiens
tacctgctccaggtgcgggctcagaattcccatggcgaggactatgcggcccctctggagctgcacgtgctggtgatggatgagaatgacaacgtgcctatctgccctccccgtgaccccacagtcagcatccctgagctcagtccaccag

SEQ ID NO: 49 - DNA, 228 bp, Homo sapiens
gtactgaagtgactagactgtcagcagaggatgcagatgcccccggctcccccaattcccacgttgtgtatcagctcctgagccctgagcctgaggatggggtagaggggagagccttccaggtggaccccacttcaggcagtgtgacgctgggggtgctcccactccgagcaggccagaacatcctgcttctggtgctggccatggacctggcaggcgcagagggtg

SEQ ID NO: 50 - DNA, 189 bp, Homo sapiens
attgggcctataagcctccctgaggatgtggagcccgggactctggtggccatgctaacagccattgatgctgacctcgagcccgccttccgcctcatggattttgccattgagaggggagacacagaagggacttttggcctggattgggagccagactctgggcatgttagactcagactctgcaag

SEQ ID NO: 51 - DNA, 220 bp, Homo sapiens
gtatggccaggcagtgatgggagcccctgggagggggtacaaacctcagacagacagacagcaggactgagcttcctcccagaggccctcactggcaacccccagccccaagcaggagaggcagatatgaagctgggttcaatgttcctctctgagaatggatctactcctccggtgggcaagtctggcatctgcctcccctctctgatcattctcctag

SEQ ID NO: 52 - DNA, 459 bp, Homo sapiens
attgacacacaaaatctcccagccctgtcctttagccactggagggcctctcagggtgtcagcttcagggactggtatagggccggggaggtggcaggaggaccaggggctgcccctggggaaggagtgctggagtgaatgggccactggagccaggtgtggaggacactgaacactgctcaggacagagagctggccctggggtggggagccccgggaggcaggtttcaactctgagctgggctgtgatacagggcagacccagcagcagtgagctccgccaaagtcagggatgtccacagtggcatggggatgatgacaggtggagagaagcatggagggagcatggcaggggagtctataaggcgaggctcatgggctgcgggagcagcccagggacctcagagctcaccctccccctggcaacacctggccagctcagcactgcctcctctgcag

SEQ ID NO: 53 - DNA, 316 bp, Homo sapiens
cacactgctgtgatgagcctttctcttggaggtccatggagttgggatccaacttactggttccccgatgtccccaagagaaggggactttggaggctgagtatactgggtcagcctcagaggggtaggggccaagccggccgaggagtcaagcttctccaggcaccaccacagagtcatacccagcccagcctcaggatgctgtccaggaaactcatctcttcattaatccaatgccaatcacaggaagtccctcgtgatgaacaagtcccacagccccttctgaggcctgatacatccctcttgggccccacag

SEQ ID NO: 54 - DNA, 142 bp, Homo sapiens
gaatcttcctcatcctcattttcacccactggaccatgtcaaggaagaaggacccggatcaaccagcagacagcgtgcccctgaaggcgactgtctgaatggcccaggcagctctagctgggagcttggcctctggctccat

SEQ ID NO: 55 - DNA, 114 bp, Homo sapiens
gctcaagtgccctgccttgccccacccagcccagcctggccagagccccctggagaaggagctctcttcttgcttggcagctggaccaagggagccagtcttgggcgctggagg

SEQ ID NO: 56 - DNA, 58 bp, Homo sapiens
gcctgtcctgaccatggtccctgcctggctgtggctgctttgtgtctccgtcccccag

SEQ ID NO: 57 - DNA, 84 bp, Homo sapiens
gctctccccaaggcccagcctgcagagctgtctgtggaagttccagaaaactatggtggaaatttccctttatacctgaccaag

SEQ ID NO: 58 - DNA, 81 bp, Homo sapiens
gtcaccctggagatgcaggatggacatgtcttgtggggtccacagcctgtgcttgtgcacgtgaaggatgagaatgaccag

SEQ ID NO: 59 - DNA, 23 bp, Homo sapiens
gtgccccatttctctcaagccat

SEQ ID NO: 60 - DNA, 35 bp, Homo sapiens
ctacagagctcggctgagccggggtaccaggcctg

SEQ ID NO: 61 - DNA, 23 bp, Homo sapiens
tttgccctcccaccgtgaaccag

SEQ ID NO: 62 - DNA, 23 bp, Homo sapiens
gcatccccttcctcttccttgag

SEQ ID NO: 63 - DNA, 10 bp, Homo sapiens
ggagcaccag

SEQ ID NO: 64 - DNA, 64 bp, Homo sapiens
ccttgaccacgccctggagaggacctaccagctgttggtacaggtcaaggacatgggtgaccag

SEQ ID NO: 65 - DNA, 57 bp, Homo sapiens
gcttcagcagcacgtgtgaagtcgaagtcgcagtcacagatatcaatgatcacgccc

SEQ ID NO: 66 - DNA, 20 bp, Homo sapiens
ctgagttcatcacttcccag

SEQ ID NO: 67 - DNA, 71 bp, Homo sapiens
aacctcagttatgaggcagctccaagtcatgaggtggtggtggtggtgcagagtgtggcgaagctggtggg

SEQ ID NO: 68 - DNA, 11 bp, Homo sapiens
gccaggcccag

SEQ ID NO: 69 - DNA, 3 bp, Homo sapiens
gcc

SEQ ID NO: 70 - DNA, 74 bp, Homo sapiens
ctggagccaccgccacggtgactgtgctagtggagagagtgatgccaccccccaagttggaccaggagagctac

SEQ ID NO: 71 - DNA, 3 bp, Homo sapiens
gag

SEQ ID NO: 72 - DNA, 80 bp, Homo sapiens
gccagtgtccccatcagtgccccagccggctctttcctgctgaccatccagccctccgaccccatcagccgaaccctcag

SEQ ID NO: 73 - DNA, 102 bp, Homo sapiens
gttctccctagtcaatgactcagagggctggctctgcattgagaaattctccggggaggtgcacaccgcccagtccctgcagggcgcccagcctggggacac

SEQ ID NO: 74 - DNA, 32 bp, Homo sapiens
ctacacggtgcttgtggaggcccaggatacag

SEQ ID NO: 75 - DNA, 66 bp, Homo sapiens
atgagccgagactgagcgcttctgcacccctggtgatccacttcctaaaggcccctcctgccccag

SEQ ID NO: 76 - DNA, 34 bp, Homo sapiens
ccctgactcttgcccctgtgccctcccaatacct

SEQ ID NO: 77 - DNA, 17 bp, Homo sapiens
ctgcacaccccgccaag

SEQ ID NO: 78 - DNA, 24 bp, Homo sapiens
accatggcttgatcgtgagtggac

SEQ ID NO: 79 - DNA, 1 bp, Homo sapiens
c

SEQ ID NO: 80 - DNA, 3 bp, Homo sapiens
cag

SEQ ID NO: 81 - DNA, 98 bp, Homo sapiens
caaggaccccgatctggccagtgggcacggtccctacagcttcacccttggtcccaaccccacggtgcaacgggattggcgcctccagactctcaatg

SEQ ID NO: 82 - DNA, 5 bp, Homo sapiens
gttcc

SEQ ID NO: 83 - DNA, 103 bp, Homo sapiens
catgcctacctcaccttggccctgcattgggtggagccacgtgaacacataatccccgtggtggtcagccacaatgcccagatgtggcagctcctggttcgag

SEQ ID NO: 84 - DNA, 117 bp, Homo sapiens
tgatcgtgtgtcgctgcaacgtggaggggcagtgcatgcgcaaggtgggccgcatgaagggcatgcccacgaagctgtcggcagtgggcatccttgtaggcaccctggtagcaatag

SEQ ID NO: 85 - DNA, 71 bp, Homo sapiens
ctgagtcccctgggagagagcccagcacccaagatccagcaggggacaggacagagtagaagcccctccat

SEQ ID NO: 86 - DNA, 41 bp, Homo sapiens
ctgccctggggtggaggcaccatcaccatcaccaggcatgt

SEQ ID NO: 87 - DNA, 17 bp, Homo sapiens
ctgcagagcctggacac

SEQ ID NO: 88 - DNA, 97 bp, Homo sapiens
caactttatggactgcccatgggagtgctccaaatgtcagggtgtttgcccaataataaagccccagagaactgggctgggccctatgggattggta

SEQ ID NO: 89 - protein, 829 aa, Homo sapiens
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPGIPFLFLEASDRDEPGTANSDLRFHILSQAPAQPSPDMFQLEPRLGALALSPKGSTSLDHALERTYQLLVQVKDMGDQASGHQATATVEVSIIESTWVSLEPIHLAENLKVLYPHHMAQVHWSGGDVHYHLESHPPGPFEVNAEGNLYVTRELDREAQAEYLLQVRAQNSHGEDYAAPLELHVLVMDENDNVPICPPRDPTVSIPELSPPGTEVTRLSAEDADAPGSPNSHVVYQLLSPEPEDGVEGRAFQVDPTSGSVTLGVLPLRAGQNILLLVLAMDLAGAEGGFSSTCEVEVAVTDINDHAPEFITSQIGPISLPEDVEPGTLVAMLTAIDADLEPAFRLMDFAIERGDTEGTFGLDWEPDSGHVRLRLCKNLSYEAAPSHEVVVVVQSVAKLVGPGPGPGATATVTVLVERVMPPPKLDQESYEASVPISAPAGSFLLTIQPSDPISRTLRFSLVNDSEGWLCIEKFSGEVHTAQSLQGAQPGDTYTVLVEAQDTDEPRLSASAPLVIHFLKAPPAPALTLAPVPSQYLCTPRQDHGLIVSGPSKDPDLASGHGPYSFTLGPNPTVQRDWRLQTLNGSHAYLTLALHWVEPREHIIPVVVSHNAQMWQLLVRVIVCRCNVEGQCMRKVGRMKGMPTKLSAVGILVGTLVAIGIFLILIFTHWTMSRKKDPDQPADSVPLKATV

SEQ ID NO: 90 - protein, 807 aa, Homo sapiens
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPGIPFLFLEASDRDEPGTANSDLRFHILSQAPAQPSPDMFQLEPRLGALALSPKGSTSLDHALERTYQLLVQVKDMGDQASGHQATATVEVSIIESTWVSLEPIHLAENLKVLYPHHMAQVHWSGGDVHYHLESHPPGPFEVNAEGNLYVTRELDREAQAEYLLQVRAQNSHGEDYAAPLELHVLVMDENDNVPICPPRDPTVSIPELSPPGTEVTRLSAEDADAPGSPNSHVVYQLLSPEPEDGVEGRAFQVDPTSGSVTLGVLPLRAGQNILLLVLAMDLAGAEGGFSSTCEVEVAVTDINDHAPEFITSQIGPISLPEDVEPGTLVAMLTAIDADLEPAFRLMDFAIERGDTEGTFGLDWEPDSGHVRLRLCKNLSYEAAPSHEVVVVVQSVAKLVGPGPGPGATATVTVLVERVMPPPKLDQESYEASVPISAPAGSFLLTIQPSDPISRTLRFSLVNDSEGWLCIEKFSGEVHTAQSLQGAQPGDTYTVLVEAQDTALTLAPVPSQYLCTPRQDHGLIVSGPSKDPDLASGHGPYSFTLGPNPTVQRDWRLQTLNGSHAYLTLALHWVEPREHIIPVVVSHNAQMWQLLVRVIVCRCNVEGQCMRKVGRMKGMPTKLSAVGILVGTLVAIGIFLILIFTHWTMSRKKDPDQPADSVPLKATV

SEQ ID NO: 91 - protein, 646 aa, Homo sapiens
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPGIPFLFLEASDRDEPGTANSDLRFHILSQAPAQPSPDMFQLEPRLGALALSPKGSTSLDHALERTYQLLVQVKDMGDQASGHQATATVEVSIIESTWVSLEPIHLAENLKVLYPHHMAQVHWSGGDVHYHLESHPPGPFEVNAEGNLYVTRELDREAQAEYLLQVRAQNSHGEDYAAPLELHVLVMDENDNVPICPPRDPTVSIPELSPPGTEVTRLSAEDADAPGSPNSHVVYQLLSPEPEDGVEGRAFQVDPTSGSVTLGVLPLRAGQNILLLVLAMDLAGAEGGFSSTCEVEVAVTDINDHAPEFITSQIGPISLPEDVEPGTLVAMLTAIDADLEPAFRLMDFAIERGDTEGTFGLDWEPDSGHVRLRLCKNLSYEAAPSHEVVVVVQSVAKLVGPGPGPGATATVTVLVERVMPPPKLDQESYEASVPISAPAGSFLLTIQPSDPISRTLRFSLVNDSEGWLCIEKFSGEVHTAQSLQGAQPGDTYTVLVEAQDTGMARQ

SEQ ID NO: 92 - protein, 175 aa, Homo sapiens
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPVCPPTVNQASPSSSLRLQTGMSQAQPTRIFDSTS

SEQ ID NO: 93 - protein, 303 aa, Homo sapiens
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPGIPFLFLEASDRDEPGTANSDLRFHILSQAPAQPSPDMFQLEPRLGALALSPKGSTSLDHALERTYQLLVQVKDMGDQASGHQATATVEVSIIESTWVSLEPIHLAENLKVLYPHHMAQVHWSGGDVHYHLESHPPGPFEVNAEGNLYVTRELDREAQAEVI

SEQ ID NO: 94 - protein, 230 aa, Homo sapiens [sequence omitted]

SEQ ID NO: 95 - protein, 146 aa, Homo sapiens
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPGFRPG

SEQ ID NO: 96 - protein, 839 aa, Homo sapiens
MVPAWLWLLCVSVPQALPKAQPAELSVEVPENYGGNFPLYLTKLPLPREGAEGQIVLSGDSGKATEGPFAMDPDSGFLLVTRALDREEQAEYQLQVTLEMQDGHVLWGPQPVLVHVKDENDQVPHFSQAIYRARLSRGTRPGIPFLFLEASDRDEPGTANSDLRFHILSQAPAQPSPDMFQLEPRLGALALSPKGSTSLDHALERTYQLLVQVKDMGDQASGHQATATVEVSIIESTWVSLEPIHLAENLKVLYPHHMAQVHWSGGDVHYHLESHPPGPFEVNAEGNLYVTRELDREAQAEYLLQVRAQNSHGEDYAAPLELHVLVMDENDNVPICPPRDPTVSIPELSPPGTEVTRLSAEDADAPGSPNSHVVYQLLSPEPEDGVEGRAFQVDPTSGSVTLGVLPLRAGQNILLLVLAMDLAGAEGGFSSTCEVEVAVTDINDHAPEFITSQIGPISLPEDVEPGTLVAMLTAIDADLEPAFRLMDFAIERGDTEGTFGLDWEPDSGHVRLRLCKNLSYEAAPSHEVVVVVQSVAKLVGPGPGPGATATVTVLVERVMPPPKLDQESYEASVPISAPAGSFLLTIQPSDPISRTLRFSLVNDSEGWLCIEKFSGEVHTAQSLQGAQPGDTYTVLVEAQDTDEPRLSASAPLVIHFLKAPPAPALTLAPVPSQYLCTPRQDHGLIVSGPSKDPDLASGHGPYSFTLGPNPTVQRDWRLQTLNGSHAYLTLALHWVEPREHIIPVVVSHNAQMWQLLVRVIVCRCNVEGQCMRKVGRMKGMPTKLSAVGILVGTLVAIAHCCDEPFSWRSMELGSNLLVPRCPQEKGTLEAEYTGSASEG

SEQ ID NO: 97 - DNA, 22 bp, Artificial Sequence
  Synthetic oligonucleotide
gtgcttggtcagtgtccagttg

SEQ ID NO: 98 - DNA, 21 bp, Artificial Sequence
  Synthetic oligonucleotide
gctctgagtttccttccggtg

SEQ ID NO: 99 - DNA, 115 bp, Artificial Sequence
  Synthetic oligonucleotide
gtgcttggtcagtgtccagttggcttttgggggtgtcacaactacaacagaggctgaggcagtgggaaagagccccaaagagctggagctggcccaccggaaggaaactcagagc

SEQ ID NO: 100 - DNA, 21 bp, Artificial Sequence
  Synthetic oligonucleotide
ggactgagcttcctcccagag

SEQ ID NO: 101 - DNA, 20 bp, Artificial Sequence
  Synthetic oligonucleotide
cccaccggaggagtagatcc

SEQ ID NO: 102 - DNA, 116 bp, Artificial Sequence
  Synthetic oligonucleotide
ggactgagcttcctcccagaggccctcactggcaacccccagccccaagcaggagaggcagatatgaagctgggttcaatgttcctctctgagaatggatctactcctccggtggg

SEQ ID NO: 103 - DNA, 20 bp, Artificial Sequence
  Synthetic oligonucleotide
catgcctacctcaccttggc

SEQ ID NO: 104 - DNA, 18 bp, Artificial Sequence
  Synthetic oligonucleotide
ctcgaaccaggagctgcc

SEQ ID NO: 105 - DNA, 1282 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggtccccaacctgccctgtccatccccctcccagactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 106 - DNA, 1351 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatggtgaggccagaggcgggcagtggggcggtggggggcggtgggggacggtggggggcggtggggcagaggcagtgcctgctggtgacaccctctccccacagagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 107 - DNA, 1335 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggtaggtggggggaggggcaacgcagcccgtccaccaggcccggtcctgcagccccaagtgagccgctgggtggccttgcccgcaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 108 - DNA, 1328 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacaggtgctccctgctgctgagggctgggcccccttccctgggcggctgctgagccctcctcaccctggtatctcactgcagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 109 - DNA, 1353 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaagtacgccccgctacccgccgaggggtgttgtgggagtgaaagagggggttgggtggggggcctgggggttcttcacggggctcaccgcccctccctgctgcagcaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 110 - DNA, 1236 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctggcaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 111 - DNA, 1205 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 112 - DNA, 1246 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 113 - DNA, 1367 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggtccccaacctgccctgtccatccccctcccagactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggtaggtggggggaggggcaacgcagcccgtccaccaggcccggtcctgcagccccaagtgagccgctgggtggccttgcccgcaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 114 - DNA, 1429 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacaggtgctccctgctgctgagggctgggcccccttccctgggcggctgctgagccctcctcaccctggtatctcactgcagcacagagctcgggatgtatcagccccactgtgggctcgacagggtcctcatgtcctggggccatgatggtgaggccagaggcgggcagtggggcggtggggggcggtgggggacggtggggggcggtggggcagaggcagtgcctgctggtgacaccctctccccacagagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 115 - DNA, 1808 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctggcaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctgaagccccactgggtgttactgcagcagggcgtggcccaggccgagggatggtgccagcagggtggaggccaagtcccaggctttcaggtgtgtgtgtcccaccacactggccgtggtaaacatctgcccatcctcccagctcctgtgtcagagcctgggggacgagtcacccagctcacccccacatgctcaaaacagtcaccaagtcctctgggctccgtccccttaaaaaataccccaatagctaaaataatacattcatagaaaacaacaaaaaacaaatgcaggctgggcacagcggctcacacctataatcccagcactttgggagggcgaggtgggcggatcacctgaggtcaggggttcaagaccagcctggccaacatggtgaaaccccgtctctactaaaaatacaaaaattagccgggcgtggtggtgggcgcctgtaatcccagctattcaggaggctaaggcaggagaatcgcttgaacccgggagacaggggttgccgtgagtcaagattgtgccactgcactccaacctgggtatcaagagtgaaattccatctcaaaa

SEQ ID NO: 116 - DNA, 1243 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacgggcccagacccttccctggtctaccgacctgatgtggacccagaggtggccaaagacaaggccagcttccggaactacacggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 117 - DNA, 1169 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgagtacatgtaccaggtgatgaagtttaacaagttctcactgccccctgaggctttctacatgatccggttccactccttctacccctggcacacgggccgcgactaccagcagctgtgcagccagcaggacctggccatgctgccctgggtgcgggagttcaacaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 118 - DNA, 1119 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggtccccaacctgccctgtccatccccctcccagactggttccacctcgtcgggctcctgcacgacctggggaaggtcctggccctgttcggggagccccagtgggctgtcgtcggcgacaccttccccgtcggatgccgtccgcaggcctccgtggttttctgcgactccaccttccaggacaaccctgacctccaggatcctcgatacagcacagagctcgggatgtatcagccccactgtgggctcgacagggtcctcatgtcctggggccatgatgcaagttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctga

SEQ ID NO: 119 - DNA, 721 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacggtgggcccagacccttccctggtctaccgacctgatgtggacccagaggtggccaaagacaaggccagcttccggaactacacgtcaggtcccctcctggaccgtgtcttcaccacctacaagctcatgcacacgcaccagacagtggacttcgtcaggagcaagcatgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaaggactggttccacctcgtcgggctcctgcacgacctggggaaggtcctggccctgttcggggagccccagtgggcagtcatcggcaacatcttcccccactccccccatggccgcccgtccctcctgtccctctgcagtgggcagtcatcggtgacaccttcccccactccccccatggccgccc

SEQ ID NO: 120 - DNA, 210 bp, Homo sapiens
ggctgagtcagggccagaggatgtgggactgctgggggctgctgctgtgctgacacattaactccctgaggccgggaggagggtggatggtcatttattactgcggacacttatgtaacctggggtgtatataggagcctggctgaggacacactcgccagcctgtgctttgccacctgagcgccgctccctctcaggatgaaggtgacg

SEQ ID NO: 121 - DNA, 163 bp, Homo sapiens
catgcccagtttgggggcttctcctacaagaaaatgacagtcatggaggccgtggacctgctggatgggctggtggatgagtcggacccggacgtagatttccccaactccttccatgccttccagacagcggagggcatccggaaggcccacccagacaagg

SEQ ID NO: 122 - DNA, 872 bp, Homo sapiens
gttcgacctctacaccaagtgcccggacctgccggacgtggacaagctgcggccctactaccaggggctcattgacaagtactgccctggcatcctgagctggtgaccctcctgccacccaagctgctgctggacctaggcctggccctccgcctgcctggagaggcctggccctgggcaaacagccgccatcagggttcacctcggtgggggaccccactcacccccttagggtcgccacccctcacggcaacttgtgcctggcgtcaataaagacctggaaggatgttgtgcttctgaagccccactgggtgttactgcagcagggcgtggcccaggccgagggatggtgccagcagggtggaggccaagtcccaggctttcaggtgtgtgtgtcccaccacactggccgtggtaaacatctgcccatcctcccagctcctgtgtcagagcctgggggacgagtcacccagctcacccccacatgctcaaaacagtcaccaagtcctctgggctccgtccccttaaaaaataccccaatagctaaaataatacattcatagaaaacaacaaaaaacaaatgcaggctgggcacagcggctcacacctataatcccagcactttgggagggcgaggtgggcggatcacctgaggtcaggggttcaagaccagcctggccaacatggtgaaaccccgtctctactaaaaatacaaaaattagccgggcgtggtggtgggcgcctgtaatcccagctattcaggaggctaaggcaggagaatcgcttgaacccgggagacaggggttgccgtgagtcaagattgtgccactgcactccaacctgggtatcaagagtgaaattccatctcaaaa

SEQ ID NO: 123 - DNA, 3 bp, Homo sapiens
gtg

SEQ ID NO: 124 - DNA, 81 bp, Homo sapiens
ggcccagacccttccctggtctaccgacctgatgtggacccagaggtggccaaagacaaggccagcttccggaactacacg

SEQ ID NO: 125 - DNA, 4 bp, Homo sapiens
tcag

SEQ ID NO: 126 - DNA, 77 bp, Homo sapiens
gtcccctcctggaccgtgtcttcaccacctacaagctcatgcacacgcaccagacagtggacttcgtcaggagcaag

SEQ ID NO: 127 - DNA, 32 bp, Homo sapiens
tccccaacctgccctgtccatccccctcccag

SEQ ID NO: 128 - DNA, 68 bp, Homo sapiens
actggttccacctcgtcgggctcctgcacgacctggggaaggtcctggccctgttcggggagccccag

SEQ ID NO: 129 - DNA, 115 bp, Homo sapiens
tgggcagtcatcggcaacatcttcccccactccccccatggccgcccgtccctcctgtccctctgcagtgggcagtcatcggtgacaccttcccccactccccccatggccgccc

SEQ ID NO: 130 - DNA, 45 bp, Homo sapiens
tgggctgtcgtcggcgacaccttccccgtcggatgccgtccgcag

SEQ ID NO: 131 - DNA, 65 bp, Homo sapiens
gcctccgtggttttctgcgactccaccttccaggacaaccctgacctccaggatcctcgatacag

SEQ ID NO: 132 - DNA, 78 bp, Homo sapiens
gtgctccctgctgctgagggctgggcccccttccctgggcggctgctgagccctcctcaccctggtatctcactgcag

SEQ ID NO: 133 - DNA, 68 bp, Homo sapiens
cacagagctcgggatgtatcagccccactgtgggctcgacagggtcctcatgtcctggggccatgatg

SEQ ID NO: 134 - DNA, 101 bp, Homo sapiens
gtgaggccagaggcgggcagtggggcggtggggggcggtgggggacggtggggggcggtggggcagaggcagtgcctgctggtgacaccctctccccacag

SEQ ID NO: 135 - DNA, 31 bp, Homo sapiens
agtacatgtaccaggtgatgaagtttaacaa

SEQ ID NO: 136 - DNA, 19 bp, Homo sapiens
gttctcactgccccctgag

SEQ ID NO: 137 - DNA, 85 bp, Homo sapiens
gtaggtggggggaggggcaacgcagcccgtccaccaggcccggtcctgcagccccaagtgagccgctgggtggccttgcccgcag

SEQ ID NO: 138 - DNA, 46 bp, Homo sapiens
gctttctacatgatccggttccactccttctacccctggcacacgg

SEQ ID NO: 139 - DNA, 53 bp, Homo sapiens
gccgcgactaccagcagctgtgcagccagcaggacctggccatgctgccctgg

SEQ ID NO: 140 - DNA, 14 bp, Homo sapiens
gtgcgggagttcaa

SEQ ID NO: 141 - DNA, 103 bp, Homo sapiens
gtacgccccgctacccgccgaggggtgttgtgggagtgaaagagggggttgggtggggggcctgggggttcttcacggggctcaccgcccctccctgctgcag

SEQ ID NO: 142 - DNA, 3 bp, Homo sapiens
caa

SEQ ID NO: 143 - protein, 285 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKDWFHLVGLLHDLGKVLALFGEPQWAVVGDTFPVGCRPQASVVFCDSTFQDNPDLQDPRYSTELGMYQPHCGLDRVLMSWGHDEYMYQVMKFNKFSLPPEAFYMIRFHSFYPWHTGRDYQQLCSQQDLAMLPWVREFNKFDLYTKCPDLPDVDKLRPYYQGLIDKYCPGILSW

SEQ ID NO: 144 - protein, 211 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKVPNLPCPSPSQTGSTSSGSCTTWGRSWPCSGSPSGLSSATPSPSDAVRRPPWFSATPPSRTTLTSRILDTAQSSGCISPTVGSTGSSCPGAMMSTCTR

SEQ ID NO: 145 - protein, 234 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKDWFHLVGLLHDLGKVLALFGEPQWAVVGDTFPVGCRPQASVVFCDSTFQDNPDLQDPRYSTELGMYQPHCGLDRVLMSWGHDGEARGGQWGGGGRWGTVGGGGAEAVPAGDTLSPQSTCTR

SEQ ID NO: 146 - protein, 303 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKDWFHLVGLLHDLGKVLALFGEPQWAVVGDTFPVGCRPQASVVFCDSTFQDNPDLQDPRYSTELGMYQPHCGLDRVLMSWGHDEYMYQVMKFNKFSLPPEVGGGRGNAARPPGPVLQPQVSRWVALPAGFLHDPVPLLLPLAHGPRLPAAVQPAGPGHAALGAGVQQVRPLHQVPGPAGRGQAAALLPGAH

SEQ ID NO: 147 - protein, 188 aa, Homo sapiens [sequence omitted]

SEQ ID NO: 148 - protein, 309 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKDWFHLVGLLHDLGKVLALFGEPQWAVVGDTFPVGCRPQASVVFCDSTFQDNPDLQDPRYSTELGMYQPHCGLDRVLMSWGHDEYMYQVMKFNKFSLPPEAFYMIRFHSFYPWHTGRDYQQLCSQQDLAMLPWVREFKYAPLPAEGCCGSERGGWVGGLGVLHGAHRPSLLQQVRPLHQVPGPAGRGQAAALLPGAH

SEQ ID NO: 149 - protein, 270 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKDWFHLVGLLHDLGKVLALFGEPQWAVVGDTFPVGCRPQASVVFCDSTFQDNPDLQDPRYSTELGMYQPHCGLDRVLMSWGHDEYMYQVMKFNKFSLPPEAFYMIRFHSFYPWHTGRDYQQLCSQQDLAMLPWQVRPLHQVPGPAGRGQAAALLPGAH

SEQ ID NO: 150 - protein, 270 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKDWFHLVGLLHDLGKVLALFGEPQASVVFCDSTFQDNPDLQDPRYSTELGMYQPHCGLDRVLMSWGHDEYMYQVMKFNKFSLPPEAFYMIRFHSFYPWHTGRDYQQLCSQQDLAMLPWVREFNKFDLYTKCPDLPDVDKLRPYYQGLIDKYCPGILSW

SEQ ID NO: 151 - protein, 258 aa, Homo sapiens
MKVTVGPDPSLVYRPDVDPEVAKDKASFRNYTHAQFGGFSYKKMTVMEAVDLLDGLVDESDPDVDFPNSFHAFQTAEGIRKAHPDKDWFHLVGLLHDLGKVLALFGEPQWAVVGDTFPVGCRPQASVVFCDSTFQDNPDLQDPRYSTELGMYQPHCGLDRVLMSWGHDEYMYQVMKFNKFSLPPEAFYMIRFHSFYPWHTGRDYQQLCSQQDLAMLPWVREFNKFDLYTKCPDLPDVDKLRPYYQGLIDKYCPGILSW

SEQ ID NO: 152 - protein, 231 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKVPNLPCPSPSQTGSTSSGSCTTWGRSWPCSGSPSGLSSATPSPSDAVRRPPWFSATPPSRTTLTSRILDTAQSSGCISPTVGSTGSSCPGAMMQVRPLHQVPGPAGRGQAAALLPGAH

SEQ ID NO: 153 - protein, 174 aa, Homo sapiens
MKVTVGPDPSLVYRPDVDPEVAKDKASFRNYTSGPLLDRVFTTYKLMHTHQTVDFVRSKHAQFGGFSYKKMTVMEAVDLLDGLVDESDPDVDFPNSFHAFQTAEGIRKAHPDKDWFHLVGLLHDLGKVLALFGEPQWAVIGNIFPHSPHGRPSLLSLCSGQSSVTPSPTPPMAA

SEQ ID NO: 154 - protein, 69 aa, Homo sapiens
MKVTVGPDPSLVYRPDVDPEVAKDKASFRNYTVPSWTVSSPPTSSCTRTRQWTSSGASMPSLGASPTRK

SEQ ID NO: 155 - protein, 68 aa, Homo sapiens
MKVTGPDPSLVYRPDVDPEVAKDKASFRNYTVPSWTVSSPPTSSCTRTRQWTSSGASMPSLGASPTRK

SEQ ID NO: 156 - DNA, 16 bp, Artificial Sequence
  Synthetic oligonucleotide
gggagccccaggcctc

SEQ ID NO: 157 - DNA, 21 bp, Artificial Sequence
  Synthetic oligonucleotide
cacagtggggctgatacatcc

SEQ ID NO: 158 - DNA, 108 bp, Artificial Sequence
  Synthetic oligonucleotide
gggagccccaggcctccgtggttttctgcgactccaccttccaggacaaccctgacctccaggatcctcgatacagcacagagctcgggatgtatcagccccactgtg

SEQ ID NO: 159 - DNA, 19 bp, Artificial Sequence
  Synthetic oligonucleotide
tgacagtcatggaggccgt

SEQ ID NO: 160 - DNA, 18 bp, Artificial Sequence
  Synthetic oligonucleotide
ctccgctgtctggaaggc

SEQ ID NO: 161 - DNA, 101 bp, Artificial Sequence
  Synthetic oligonucleotide
tgacagtcatggaggccgtggacctgctggatgggctggtggatgagtcggacccggacgtagatttccccaactccttccatgccttccagacagcggag

SEQ ID NO: 162 - DNA, 20 bp, Artificial Sequence
  Synthetic oligonucleotide
tcctcgatacaggtgctccc

SEQ ID NO: 163 - DNA, 19 bp, Artificial Sequence
  Synthetic oligonucleotide
ccgagctctgtgctgcagt

SEQ ID NO: 164 - DNA, 102 bp, Artificial Sequence
  Synthetic oligonucleotide
tcctcgatacaggtgctccctgctgctgagggctgggcccccttccctgggcggctgctgagccctcctcaccctggtatctcactgcagcacagagctcgg

SEQ ID NO: 165 - DNA, 1768 bp, Homo sapiens
cgcggacccgcagcgactccacagggactgcgctcccttgcccctagcgctcccgcgctgctgctccagccgcccggcagctctgaggatggagaggagggcgcggagctcctccagggagtcccgcgggcgaggcggcaggactccgcacaaggagaacaagagggcaaaggccgagaggagcggcgggggccgcgggcgccaggaggctgggcccgagccgtcgggctccggacgggcggggaccccgggggagccccgagcgcccgccgccacggtggtggacgtggatgaggtccgaggctccggcgaggagggcaccgaggtggtggcgctgttggagagcgagcggcccgaggaaggttgtacaagagtatgaaagagtaattatattccgactgggacatctgcttcctggaagagccaaaggccctggtcttttcttttttttgccctgcctggatacctaccacaaggttgaccttcgtctccaaactctggagataccttttcatgagatcgtgaccaaagacatgtttataatggagatagatgccatttgctactaccgaatggaaaatgcctctcttctcctaagcagtcttgctcatgtatctaaagctgtgcaattccttgtgcaaaccactatgaagcgtctcctagcacatcgatccctcactgaaattcttctagagaggaagagcatcgcccaagatgcaaaggttgccttggattcagtgacctgtatttggggaatcaaagtggagagaatagaaattaaagatgtgaggttgccagctgggcttcagcactcactggctgtggaggctgaagcgcaaagacaagccaaagtgcggatgattgctgcagaagcggaaaaggctgcttctgagtccctgaggatggcagctgagattctgtcaggcacccctgctgctgttcagcttcgatacctccacacccttcagtctctgtccacagagaagccttccactgtggttttacctttgccatttgacctactgaattgcctgtcttctcccagcaacagaactcagggaagcctccccttcccaagtccttccaaacctgttgagccactaaatcctaaaaagaaagactctcccatgttataggaaggatggggcataatgtgactgtaaaggggcctgccatagaaaagtcacatccctgagggagacactctgtcctcattccctgcccttcctttggttgccatatggaatggccatggaatgcacgaagtcacaatgcaccatccatgagaagacagtgaaatgatgtaatgacagagaaggcagacaacatgtttccgtgactcatctagtcagagcaattatgggaaacagctttggtcaacattctactttggaaagaattttgagtctagatgtggttaaattttgacttctgggaacttggttcagatgtccctttcactgtatgtcctctgacccctttggcaaggttgccacagctcccacagcccttcctacaagcacctatcattgggcttgtcacactctattgctcttctgtcccgaagatgcagtcttctctccaatgatactaccaagtcttagttttcctcaaccacactcaatctttttgctccaccctgaattcctcacacctaaccctgatagttacctaaagtgacacttaaatgtttcagagtgaatgcaaaaaagagagatgtacttggagtcggatatacaatttatccctaattaaagcatttaaaagg

SEQ ID NO: 166 - DNA, 1695 bp, Homo sapiens
cgcggacccgcagcgactccacagggactgcgctcccttgcccctagcgctcccgcgctgctgctccagccgcccggcagctctgaggatggagaggagggcgcggagctcctccagggagtcccgcgggcgaggcggcaggactccgcacaaggagaacaagagggcaaaggccgagaggagcggcgggggccgcgggcgccaggaggctgggcccgagccgtcgggctccggacgggcggggaccccgggggagccccgagcgcccgccgccacggtggtggacgtggatgaggtccgaggctccggcgaggagggcaccgaggtggtggcgctgttggagagcgagcggcccgaggaaggtcttttcttttttttgccctgcctggatacctaccacaaggttgaccttcgtctccaaactctggagataccttttcatgagatcgtgaccaaagacatgtttataatggagatagatgccatttgctactaccgaatggaaaatgcctctcttctcctaagcagtcttgctcatgtatctaaagctgtgcaattccttgtgcaaaccactatgaagcgtctcctagcacatcgatccctcactgaaattcttctagagaggaagagcatcgcccaagatgcaaaggttgccttggattcagtgacctgtatttggggaatcaaagtggagagaatagaaattaaagatgtgaggttgccagctgggcttcagcactcactggctgtggaggctgaagcgcaaagacaagccaaagtgcggatgattgctgcagaagcggaaaaggctgcttctgagtccctgaggatggcagctgagattctgtcaggcacccctgctgctgttcagcttcgatacctccacacccttcagtctctgtccacagagaagccttccactgtggttttacctttgccatttgacctactgaattgcctgtcttctcccagcaacagaactcagggaagcctccccttcccaagtccttccaaacctgttgagccactaaatcctaaaaagaaagactctcccatgttataggaaggatggggcataatgtgactgtaaaggggcctgccatagaaaagtcacatccctgagggagacactctgtcctcattccctgcccttcctttggttgccatatggaatggccatggaatgcacgaagtcacaatgcaccatccatgagaagacagtgaaatgatgtaatgacagagaaggcagacaacatgtttccgtgactcatctagtcagagcaattatgggaaacagctttggtcaacattctactttggaaagaattttgagtctagatgtggttaaattttgacttctgggaacttggttcagatgtccctttcactgtatgtcctctgacccctttggcaaggttgccacagctcccacagcccttcctacaagcacctatcattgggcttgtcacactctattgctcttctgtcccgaagatgcagtcttctctccaatgatactaccaagtcttagttttcctcaaccacactcaatctttttgctccaccctgaattcctcacacctaaccctgatagttacctaaagtgacacttaaatgtttcagagtgaatgcaaaaaagagagatgtacttggagtcggatatacaatttatccctaattaaagcatttaaaagg

SEQ ID NO: 167 - DNA, 1853 bp, Homo sapiens
cgcggacccgcagcgactccacagggactgcgctcccttgcccctagcgctcccgcgctgctgctccagccgcccggcagctctgaggatggagaggagggcgcggagctcctccagggagtcccgcgggcgaggcggcaggactccgcacaaggagaacaagagggcaaaggccgagaggagcggcgggggccgcgggcgccaggaggctgggcccgagccgtcgggctccggacgggcggggaccccgggggagccccgagcgcccgccgccacggtggtggacgtggatgaggtccgaggctccggcgaggagggcaccgaggtggtggcgctgttggagagcgagcggcccgaggaaggtcttttcttttttttgccctgcctggatacctaccacaaggttgaccttcgtctccaaactctggagataccttttcatgagatcgtgaccaaagacatgtttataatggagatagatgccatttgctactaccgaatggaaaatgcctctcttctcctaagcagtcttgctcatgtatctaaagctgtgcaattccttgtgcaaaccactatgaagcgtctcctagcacatcgatccctcactgaaattcttctagagaggaagagcatcgcccaagatgcaaaggttgccttggattcagtgacctgtatttggggaatcaaagtggagagaatagaaattaaagatgtgaggttgccagctgggcttcagcactcactggctgtggaggctgaagcgcaaagacaagccaaagtgcggctcaagacccttcagaaagaagaataaacatgttctatgcttaaccgtgcttgccacatagtagatgctcagtgcttgtctgctgagtcatactgcatagtggtgaagccttcagggaatgaagaacaatcactttgctttcgtcacatgttttctagatgattgctgcagaagcggaaaaggctgcttctgagtccctgaggatggcagctgagattctgtcaggcacccctgctgctgttcagcttcgatacctccacacccttcagtctctgtccacagagaagccttccactgtggttttacctttgccatttgacctactgaattgcctgtcttctcccagcaacagaactcagggaagcctccccttcccaagtccttccaaacctgttgagccactaaatcctaaaaagaaagactctcccatgttataggaaggatggggcataatgtgactgtaaaggggcctgccatagaaaagtcacatccctgagggagacactctgtcctcattccctgcccttcctttggttgccatatggaatggccatggaatgcacgaagtcacaatgcaccatccatgagaagacagtgaaatgatgtaatgacagagaaggcagacaacatgtttccgtgactcatctagtcagagcaattatgggaaacagctttggtcaacattctactttggaaagaattttgagtctagatgtggttaaattttgacttctgggaacttggttcagatgtccctttcactgtatgtcctctgacccctttggcaaggttgccacagctcccacagcccttcctacaagcacctatcattgggcttgtcacactctattgctcttctgtcccgaagatgcagtcttctctccaatgatactaccaagtcttagttttcctcaaccacactcaatctttttgctccaccctgaattcctcacacctaaccctgatagttacctaaagtgacacttaaatgtttcagagtgaatgcaaaaaagagagatgtacttggagtcggatatacaatttatccctaattaaagcatttaaaagg

SEQ ID NO: 168 - DNA, 218 bp, Homo sapiens
tccgcacaaggagaacaagagggcaaaggccgagaggagcggcgggggccgcgggcgccaggaggctgggcccgagccgtcgggctccggacgggcggggaccccgggggagccccgagcgcccgccgccacggtggtggacgtggatgaggtccgaggctccggcgaggagggcaccgaggtggtggcgctgttggagagcgagcggcccgaggaag

SEQ ID NO: 169 - DNA, 204 bp, Homo sapiens
atcgtgaccaaagacatgtttataatggagatagatgccatttgctactaccgaatggaaaatgcctctcttctcctaagcagtcttgctcatgtatctaaagctgtgcaattccttgtgcaaaccactatgaagcgtctcctagcacatcgatccctcactgaaattcttctagagaggaagagcatcgcccaagatgcaaag

SEQ ID NO: 170 - DNA, 158 bp, Homo sapiens
ctcaagacccttcagaaagaagaataaacatgttctatgcttaaccgtgcttgccacatagtagatgctcagtgcttgtctgctgagtcatactgcatagtggtgaagccttcagggaatgaagaacaatcactttgctttcgtcacatgttttctag

SEQ ID NO: 171 - DNA, 968 bp, Homo sapiens
atgattgctgcagaagcggaaaaggctgcttctgagtccctgaggatggcagctgagattctgtcaggcacccctgctgctgttcagcttcgatacctccacacccttcagtctctgtccacagagaagccttccactgtggttttacctttgccatttgacctactgaattgcctgtcttctcccagcaacagaactcagggaagcctccccttcccaagtccttccaaacctgttgagccactaaatcctaaaaagaaagactctcccatgttataggaaggatggggcataatgtgactgtaaaggggcctgccatagaaaagtcacatccctgagggagacactctgtcctcattccctgcccttcctttggttgccatatggaatggccatggaatgcacgaagtcacaatgcaccatccatgagaagacagtgaaatgatgtaatgacagagaaggcagacaacatgtttccgtgactcatctagtcagagcaattatgggaaacagctttggtcaacattctactttggaaagaattttgagtctagatgtggttaaattttgacttctgggaacttggttcagatgtccctttcactgtatgtcctctgacccctttggcaaggttgccacagctcccacagcccttcctacaagcacctatcattgggcttgtcacactctattgctcttctgtcccgaagatgcagtcttctctccaatgatactaccaagtcttagttttcctcaaccacactcaatctttttgctccaccctgaattcctcacacctaaccctgatagttacctaaagtgacacttaaatgtttcagagtgaatgcaaaaaagagagatgtacttggagtcggatatacaatttatccctaattaaagcatttaaaaggaattctttttgtggagattccttttttaaataaagaaataaataaaaggacaaaaac

SEQ ID NO: 172 - DNA, 111 bp, Homo sapiens
cgcggacccgcagcgactccacagggactgcgctcccttgcccctagcgctcccgcgctgctgctccagccgcccggcagctctgaggatggagaggagggcgcggagctc

SEQ ID NO: 173 - DNA, 33 bp, Homo sapiens
ctccagggagtcccgcgggcgaggcggcaggac

SEQ ID NO: 174 - DNA, 73 bp, Homo sapiens
gttgtacaagagtatgaaagagtaattatattccgactgggacatctgcttcctggaagagccaaaggccctg

SEQ ID NO: 175 - DNA, 83 bp, Homo sapiens
gtcttttcttttttttgccctgcctggatacctaccacaaggttgaccttcgtctccaaactctggagataccttttcatgag

SEQ ID NO: 176 - DNA, 56 bp, Homo sapiens
gttgccttggattcagtgacctgtatttggggaatcaaagtggagagaatagaaat

SEQ ID NO: 177 - DNA, 79 bp, Homo sapiens
taaagatgtgaggttgccagctgggcttcagcactcactggctgtggaggctgaagcgcaaagacaagccaaagtgcgg

SEQ ID NO: 178 - protein, 383 aa, Homo sapiens
MERRARSSSRESRGRGGRTPHKENKRAKAERSGGGRGRQEAGPEPSGSGRAGTPGEPRAPAATVVDVDEVRGSGEEGTEVVALLESERPEEGTKSSGLGACEWLLVLISLLFIIMTFPFSIWFCVKVVQEYERVIIFRLGHLLPGRAKGPGLFFFLPCLDTYHKVDLRLQTLEIPFHEIVTKDMFIMEIDAICYYRMENASLLLSSLAHVSKAVQFLVQTTMKRLLAHRSLTEILLERKSIAQDAKVALDSVTCIWGIKVERIEIKDVRLPAGLQHSLAVEAEAQRQAKVRMIAAEAEKAASESLRMAAEILSGTPAAVQLRYLHTLQSLSTEKPSTVVLPLPFDLLNCLSSPSNRTQGSLPFPSPSKPVEPLNPKKKDSPML

SEQ ID NO: 179 - protein, 315 aa, Homo sapiens
MERRARSSSRESRGRGGRTPHKENKRAKAERSGGGRGRQEAGPEPSGSGRAGTPGEPRAPAATVVDVDEVRGSGEEGTEVVALLESERPEEGTKSSGLGACEWLLVLISLLFIIMTFPFSIWFCVKVVQEYERVIIFRLGHLLPGRAKGPGLFFFLPCLDTYHKVDLRLQTLEIPFHEVALDSVTCIWGIKVERIEIKDVRLPAGLQHSLAVEAEAQRQAKVRMIAAEAEKAASESLRMAAEILSGTPAAVQLRYLHTLQSLSTEKPSTVVLPLPFDLLNCLSSPSNRTQGSLPFPSPSKPVEPLNPKKKDSPML

SEQ ID NO: 180 - protein, 324 aa, Homo sapiens
MERRARSSSRESRGRGGRTPHKENKRAKAERSGGGRGRQEAGPEPSGSGRAGTPGEPRAPAATVVDVDEVRGSGEEGTEVVALLESERPEEGLFFFLPCLDTYHKVDLRLQTLEIPFHEIVTKDMFIMEIDAICYYRMENASLLLSSLAHVSKAVQFLVQTTMKRLLAHRSLTEILLERKSIAQDAKVALDSVTCIWGIKVERIEIKDVRLPAGLQHSLAVEAEAQRQAKVRMIAAEAEKAASESLRMAAEILSGTPAAVQLRYLHTLQSLSTEKPSTVVLPLPFDLLNCLSSPSNRTQGSLPFPSPSKPVEPLNPKKKDSPML

SEQ ID NO: 181 - protein, 240 aa, Homo sapiens
MERRARSSSRESRGRGGRTPHKENKRAKAERSGGGRGRQEAGPEPSGSGRAGTPGEPRAPAATVVDVDEVRGSGEEGTEVVALLESERPEEGLFFFLPCLDTYHKVDLRLQTLEIPFHEIVTKDMFIMEIDAICYYRMENASLLLSSLAHVSKAVQFLVQTTMKRLLAHRSLTEILLERKSIAQDAKVALDSVTCIWGIKVERIEIKDVRLPAGLQHSLAVEAEAQRQAKVRLKTLQKEE

SEQ ID NO: 182 - protein, 269 aa, Homo sapiens
ADPQRLHRDCAPLPLALPRCCSSRPAALRMERRARSSSRESRGRGGRTPHKENKRAKAERSGGGRGRQEAGPEPSGSGRAGTPGEPRAPAATVVDVDEVRGSGEEGTEVVALLESERPEEGLFFFLPCLDTYHKVDLRLQTLEIPFHEIVTKDMFIMEIDAICYYRMENASLLLSSLAHVSKAVQFLVQTTMKRLLAHRSLTEILLERKSIAQDAKVALDSVTCIWGIKVERIEIKDVRLPAGLQHSLAVEAEAQRQAKVRLKTLQKEE

SEQ ID NO: 183 - protein, 96 aa, Homo sapiens
MERRARSSSRESRGRGGRTPHKENKRAKAERSGGGRGRQEAGPEPSGSGRAGTPGEPRAPAATVVDVDEVRGSGEEGTEVVALLESERPEEGCTRV

SEQ ID NO: 184 - DNA, 18 bp, Artificial Sequence
  Synthetic oligonucleotide
ggacgtggatgaggtccg

SEQ ID NO: 185 - DNA, 27 bp, Artificial Sequence
  Synthetic oligonucleotide
ctctttcatactcttgtacaaccttcc

SEQ ID NO: 186 - DNA, 102 bp, Artificial Sequence
  Synthetic oligonucleotide
ggacgtggatgaggtccgaggctccggcgaggagggcaccgaggtggtggcgctgttggagagcgagcggcccgaggaaggttgtacaagagtatgaaagag

SEQ ID NO: 187 - DNA, 21 bp, Artificial Sequence
  Synthetic oligonucleotide
gtaccaaatcctccggcttag

SEQ ID NO: 188 - DNA, 21 bp, Artificial Sequence
  Synthetic oligonucleotide
ctttacgcagaaccagatgga

SEQ ID NO: 189 - DNA, 104 bp, Artificial Sequence
  Synthetic oligonucleotide
gtaccaaatcctccggcttaggggcctgtgagtggcttcttgtcctcatttccctgctcttcatcatcatgaccttccctttttccatctggttctgcgtaaag

SEQ ID NO: 190 - DNA, 2367 bp, Homo sapiens
agactaactctacctttctggcttcaggtgctatctagacctgaagtagcgggaagagcagaaaggatggggcagccatctctgacttggatgctgatggtggtggtggcctcttggttcatcacaactgcagccactgacacctcagaagcaagatggtgctctgaatgtcacagcaatgccacctgcacggaggatgaggccgttacgacgtgcacctgtcaggagggcttcaccggcgatggcctgacctgcgtggacctggatgagtgcgccattcctggagctcacaactgctccgccaacagcagctgcgtaaacacgccaggctccttctcctgcgtctgccccgaaggcttccgcctgtcgcccggtctcggctgcacagacgtggatgagtgcgctgagcctgggcttagccactgccacgccctggccacatgtgtcaatgtggtgggcagctacttgtgcgtatgccccgcgggctaccggggggatggatggcactgtgagtgctccccgggctcctgcgggccggggttggactgcgtgcccgagggcgacgcgctcgtgtgcgcggatccgtgtcaggcgcaccgcaccctggacgagtactggcgcagcaccgagtacggggagggctacgcctgcgacacggacctgcgcggctggtaccgcttcgtgggccagggcggtgcgcgcatggccgagacctgcgtgccagtcctgcgctgcaacacggccgcccccatgtggctcaatggcacgcatccgtccagcgacgagggcatcgtgagccgcaaggcctgcgcgcactggagcggccactgctgcctgtgggatgcgtccgtccaggtgaaggcctgtgccggcggctactacgtctacaacctgacagcgccccccgagtgtcacctggcgtactgcacagaccccagctccgtggaggggacgtgtgaggagtgcagtatagacgaggactgcaaatcgaataatggcagatggcactgccagtgcaaacaggacttcaacatcactgcaccagaacacaagcccggcccggtgagcagtccaccaatcagctcaggcctgatgaccagtccaccaatcagctcagtactccgggccaggcacaagcctcagatttctcatagatatctccctcctggagcacaggctggaatgtggggccaatgacatgaaggtgtcgctgggcaagtgccagctgaagagtctgggcttcgacaaggtcttcatgtacctgagtgacagccggtgctcgggcttcaatgacagagacaaccgggactgggtgtctgtagtgaccccagcccgggatggcccctgtgggacagtgttgacgaggaatgaaacccatgccacttacagcaacaccctctacctggcagatgagatcatcatccgtgacctcaacatcaaaatcaactttgcatgctcctaccccctggacatgaaagtcagcctgaagaccgccctacagccaatggtcagtgctctaaacatcagagtgggcgggaccggcatgttcaccgtgcggatggcgctcttccagaccccttcctacacgcagccctaccaaggctcctccgtgacactgtccactgaggcttttctctacgtgggcaccatgttggatgggggcgacctgtcccgatttgcactgctcatgaccaactgctatgccacacccagtagcaatgccacggaccccctgaagtacttcatcatccaggacagatgcccacacactagagactcaactatccaagtggtggagaatggggagtcctcccagggccgattttccgtccagatgttccggtttgctggaaactatgacctagtctacctgcactgtgaagtctatctctgtgacaccatgaatgaaaagtgcaagcctacctgctctgggaccagattccgaagtgggagtgtcatagatcaatcccgtgtcctgaacttgggtcccatcacacggaaaggtgtccaggccacagtctcaagggcttttagcagcttggggctcctgaaagtctggctgcctctgcttctctcggccaccttgaccctgacttttcagtgactgacagcggaaagccctgtgctccatggctgccatctcacctcctgctgggcagggggcatgatgcgggccagtgctccagccacagaaaagaaagttcatgctttgttcagcctgccttcttttctcccttttaatcctggctgtcgagaaacagcctgtgtctttaaatgctgctttttctcaaaatgggacttgtgacggtgtacctgaggcccccatctccttaaagagtgtggcaaaataatgatttttaaatctca

SEQ ID NO: 191 - DNA, 2393 bp, Homo sapiens
agactaactctacctttctggcttcaggtgctatctagacctgaagtagcgggaagagcagaaaggatggggcagccatctctgacttggatgctgatggtggtggtggcctcttggttcatcacaactgcagccactgacacctcagaagcaagatggtgctctgaatgtcacagcaatgccacctgcacggaggatgaggccgttacgacgtgcacctgtcaggagggcttcaccggcgatggcctgacctgcgtggacctggatgagtgcgccattcctggagctcacaactgctccgccaacagcagctgcgtaaacacgccaggctccttctcctgcgtctgccccgaaggcttccgcctgtcgcccggtctcggctgcacagacgtggatgagtgcgctgagcctgggcttagccactgccacgccctggccacatgtgtcaatgtggtgggcagctacttgtgcgtatgccccgcgggctaccggggggatggatggcactgtgagtgctccccgggctcctgcgggccggggttggactgcgtgcccgagggcgacgcgctcgtgtgcgcggatccgtgtcaggcgcaccgcaccctggacgagtactggcgcagcaccgagtacggggagggctacgcctgcgacacggacctgcgcggctggtaccgcttcgtgggccagggcggtgcgcgcatggccgagacctgcgtgccagtcctgcgctgcaacacggccgcccccatgtggctcaatggcacgcatccgtccagcgacgagggcatcgtgagccgcaaggcctgcgcgcactggagcggccactgctgcctgtgggatgcgtccgtccaggtgaaggcctgtgccggcggctactacgtctacaacctgacagcgccccccgagtgtcacctggcgtactgcacagaccccagctccgtggaggggacgtgtgaggagtgcagtatagacgaggactgcaaatcgaataatggcagatggcactgccagtgcaaacaggacttcaacatcactgatatctccctcctggagcacaggctggaatgtggggccaatgacatgaaggtgtcgctgggcaagtgccagctgaagagtctgggcttcgacaaggtcttcatgtacctgagtgacagccggtgctcgggcttcaatgacagagacaaccgggactgggtgtctgtagtgaccccagcccgggatggcccctgtgggacagtgttgacgaggaatgaaacccatgccacttacagcaacaccctctacctggcagatgagatcatcatccgtgacctcaacatcaaaatcaactttgcatgctcctaccccctggacatgaaagtcagcctgaagaccgccctacagccaatggtcagccagactcaggaaaggatgctgggcactgatgagggtgcacagctgacttggaggaagaagagtgctcattgcaagctgttggctggaacctcacctggcctagcagtgtcctggagcaatgtggccccgtcctcggcctgtgctctaaacatcagagtgggcgggaccggcatgttcaccgtgcggatggcgctcttccagaccccttcctacacgcagccctaccaaggctcctccgtgacactgtccactgaggcttttctctacgtgggcaccatgttggatgggggcgacctgtcccgatttgcactgctcatgaccaactgctatgccacacccagtagcaatgccacggaccccctgaagtacttcatcatccaggacagatgcccacacactagagactcaactatccaagtggtggagaatggggagtcctcccagggccgattttccgtccagatgttccggtttgctggaaactatgacctagtctacctgcactgtgaagtctatctctgtgacaccatgaatgaaaagtgcaagcctacctgctctgggaccagattccgaagtgggagtgtcatagatcaatcccgtgtcctgaacttgggtcccatcacacggaaaggtgtccaggccacagtctcaagggcttttagcagcttggggctcctgaaagtctggctgcctctgcttctctcggccaccttgaccctgacttttcagtgactgacagcggaaagccctgtgctccatggctgccatctcacctcctgctgggcagggggcatgatgcgggccagtgctccagccacagaaaagaaagttcatgctttgttcagcctgccttcttttctcccttttaatcctggctgtcgagaaacagcctgtgtctttaaatgctgctttttctcaaaatgggacttgtgacggtgtacctgaggcccccatctccttaaagagtgtggcaaaataatgatttttaaatctca

SEQ ID NO: 192 - DNA, 2006 bp, Homo sapiens
agactaactctacctttctggcttcaggtgctatctagacctgaagtagcgggaagagcagaaaggatggggcagccatctctgacttggatgctgatggtggtggtggcctcttggttcatcacaactgcagccactgacacctcagaagcaagatggtgctctgaatgtcacagcaatgccacctgcacggaggatgaggccgttacgacgtgcacctgtcaggagggcttcaccggcgatggcctgacctgcgtggacctggatgagtgcgccattcctggagctcacaactgctccgccaacagcagctgcgtaaacacgccaggctccttctcctgcgtctgccccgaaggcttccgcctgtcgcccggtctcggctgcacagacgtggatgagtgcgctgagcctgggcttagccactgccacgccctggccacatgtgtcaatgtggtgggcagctacttgtgcgtatgccccgcgggctaccggggggatggatggcactgtgagtgctccccgggctcctgcgggccggggttggactgcgtgcccgagggcgacgcgctcgtgtgcgcggatccgtgtcaggcgcaccgcaccctggacgagtactggcgcagcaccgagtacggggagggctacgcctgcgacacggacctgcgcggctggtaccgcttcgtgggccagggcggtgcgcgcatggccgagacctgcgtgccagtcctgcgctgcaacacggccgcccccatgtggctcaatggcacgcatccgtccagcgacgagggcatcgtgagccgcaaggcctgcgcgcactggagcggccactgctgcctgtgggatgcgtccgtccaggtgaaggcctgtgccggcggctactacgtctacaacctgacagcgccccccgagtgtcacctggcgtactgcacagaccccagctccgtggaggggacgtgtgaggagtgcagtatagacgaggactgcaaatcgaataatggcagatggcactgccagtgcaaacaggacttcaacatcactgatatctccctcctggagcacaggctggaatgtggggccaatgacatgaaggtgtcgctgggcaagtgccagctgaagagtctgggcttcgacaaggtcttcatgtacctgagtgacagccggtgctcgggcttcaatgacagagacaaccgggactgggtgtctgtagtgaccccagcccgggatggcccctgtgggacagtgttgacgaggaatgaaacccatgccacttacagcaacaccctctacctggcagatgagatcatcatccgtgacctcaacatcaaaatcaactttgcatgctcctaccccctggacatgaaagtcagcctgaagaccgccctacagccaatggtcagatgcccacacactagagactcaactatccaagtggtggagaatggggagtcctcccagggccgattttccgtccagatgttccggtttgctggaaactatgacctagtctacctgcactgtgaagtctatctctgtgacaccatgaatgaaaagtgcaagcctacctgctctgggaccagattccgaagtgggagtgtcatagatcaatcccgtgtcctgaacttgggtcccatcacacggaaaggtgtccaggccacagtctcaagggcttttagcagcttggggctcctgaaagtctggctgcctctgcttctctcggccaccttgaccctgacttttcagtgactgacagcggaaagccctgtgctccatggctgccatctcacctcctgctgggcagggggcatgatgcgggccagtgctccagccacagaaaagaaagttcatgctttgttcagcctgccttcttttctcccttttaatcctggctgtcgagaaacagcctgtgtctttaaatgctgctttttctcaaaatgggacttgtgacggtgtacctgaggcccccatctccttaaagagtgtggcaaaataatgatttttaaatctca

SEQ ID NO: 193 - DNA, 2369 bp, Homo sapiens
agactaactctacctttctggcttcaggtgctatctagacctgaagtagcgggaagagcagaaaggatggggcagccatctctgacttggatgctgatggtggtggtggcctcttggttcatcacaactgcagccactgacacctcagaagcaagatggtgctctgaatgtcacagcaatgccacctgcacggaggatgaggccgttacgacgtgcacctgtcaggagggcttcaccggcgatggcctgacctgcgtggacctggatgagtgcgccattcctggagctcacaactgctccgccaacagcagctgcgtaaacacgccaggctccttctcctgcgtctgccccgaaggcttccgcctgtcgcccggtctcggctgcacagacgtggatgagtgcgctgagcctgggcttagccactgccacgccctggccacatgtgtcaatgtggtgggcagctacttgtgcgtatgccccgcgggctaccggggggatggatggcactgtgagtgctccccgggctcctgcgggccggggttggactgcgtgcccgagggcgacgcgctcgtgtgcgcggatccgtgtcaggcgcaccgcaccctggacgagtactggcgcagcaccgagtacggggagggctacgcctgcgacacggacctgcgcggctggtaccgcttcgtgggccagggcggtgcgcgcatggccgagacctgcgtgccagtcctgcgctgcaacacggccgcccccatgtggctcaatggcacgcatccgtccagcgacgagggcatcgtgagccgcaaggcctgcgcgcactggagcggccactgctgcctgtgggatgcgtccgtccaggtgaaggcctgtgccggcggctactacgtctacaacctgacagcgccccccgagtgtcacctggcgtactgcacagaccccagctccgtggaggggacgtgtgaggagtgcagtatagacgaggactgcaaatcgaataatggcagatggcactgccagtgcaaacaggacttcaacatcactgatatctccctcctggagcacaggctggaatgtggggccaatgacatgaaggtgtcgctgggcaagtgccagctgaagagtctgggcttcgacaaggtcttcatgtacctgagtgacagccggtgctcgggcttcaatgacagagacaaccgggactgggtgtctgtagtgaccccagcccgggatggcccctgtgggacagtgttgacgaggaatgaaacccatgccacttacagcaacaccctctacctggcagatgagatcatcatccgtgacctcaacatcaaaatcaactttgcatgctcctaccccctggacatgaaagtcagcctgaagaccgccctacagccaatggtcaggtgtggccagagagggtccctagggcccctagatggttctaaccccaaaccccttaaccatgagcttccctgtcaactgccacccacagggagctgggagtgagggctgggaatcagggttgcccaatggaagagccaggaattctggagcccaggttcaaatctagactttgtcataaatgatggttatgccctggccagtgggggacagagtcaaagcactgcctggttcaagccccagctctgtcacttactagttgtatgagcttgagtgagttattaagcctggctttgggaaattgatagtccctctctaggtgtcaatattttcatctgtgaaatgggtttaatagtactctaagaattaaatgatatgggaggatcgcttgagcccaggaatttgaggctgcttagagctatgattgtgatgtgaatcagattcaaccattaaactgatgtagatgattcagatgtgaatcaaatattaaagtctctacttttcaaagaggcagggtggttgtgccagagagcaggaatggtctcagtccagactgctatggaatgctgggcaaatcatatttcatatttggacctcaacttctttccttctctgtgaatgaaaggaatttgattaaagaggattttaaaaattacgctccttgcaagtgtcttggtaggggccaagggaggtgccctggcagagtgcctacaggggtaaactattcatactgggtgccacgtagtattttattttattttttttaaaaggaatctgttgctaaataaaattgaaaatcttcaaataggatgaatcacaggccaatgatggaaaattatagttgtaataagaaataagatttattgattatttatttatttatttatttatttattgagacggagtctcgtactgtcgcccaggctggagttcaatggcatgatcttggctcacttcaacctccgcctcccagg

SEQ ID NO: 194 - DNA, 127 bp, Homo sapiens
gtgctatctagacctgaagtagcgggaagagcagaaaggatggggcagccatctctgacttggatgctgatggtggtggtggcctcttggttcatcacaactgcagccactgacacctcagaagcaa

SEQ ID NO: 195 - DNA, 133 bp, Homo sapiens
ctgcgggccggggttggactgcgtgcccgagggcgacgcgctcgtgtgcgcggatccgtgtcaggcgcaccgcaccctggacgagtactggcgcagcaccgagtacggggagggctacgcctgcgacacggac

SEQ ID NO: 196 - DNA, 149 bp, Homo sapiens
aggaatgaaacccatgccacttacagcaacaccctctacctggcagatgagatcatcatccgtgacctcaacatcaaaatcaactttgcatgctcctaccccctggacatgaaagtcagcctgaagaccgccctacagccaatggtcag

SEQ ID NO: 197 - DNA, 972 bp, Homo sapiens
gtgtggccagagagggtccctagggcccctagatggttctaaccccaaaccccttaaccatgagcttccctgtcaactgccacccacagggagctgggagtgagggctgggaatcagggttgcccaatggaagagccaggaattctggagcccaggttcaaatctagactttgtcataaatgatggttatgccctggccagtgggggacagagtcaaagcactgcctggttcaagccccagctctgtcacttactagttgtatgagcttgagtgagttattaagcctggctttgggaaattgatagtccctctctaggtgtcaatattttcatctgtgaaatgggtttaatagtactctaagaattaaatgatatgggaggatcgcttgagcccaggaatttgaggctgcttagagctatgattgtgatgtgaatcagattcaaccattaaactgatgtagatgattcagatgtgaatcaaatattaaagtctctacttttcaaagaggcagggtggttgtgccagagagcaggaatggtctcagtccagactgctatggaatgctgggcaaatcatatttcatatttggacctcaacttctttccttctctgtgaatgaaaggaatttgattaaagaggattttaaaaattacgctccttgcaagtgtcttggtaggggccaagggaggtgccctggcagagtgcctacaggggtaaactattcatactgggtgccacgtagtattttattttattttttttaaaaggaatctgttgctaaataaaattgaaaatcttcaaataggatgaatcacaggccaatgatggaaaattatagttgtaataagaaataagatttattgattatttatttatttatttatttatttattgagacggagtctcgtactgtcgcccaggctggagttcaatggcatgatcttggctcacttcaacctccgcctcccagg

SEQ ID NO: 198 - DNA, 141 bp, Homo sapiens
ccagactcaggaaaggatgctgggcactgatgagggtgcacagctgacttggaggaagaagagtgctcattgcaagctgttggctggaacctcacctggcctagcagtgtcctggagcaatgtggccccgtcctcggcctg

SEQ ID NO: 199 - DNA, 246 bp, Homo sapiens
tgctctaaacatcagagtgggcgggaccggcatgttcaccgtgcggatggcgctcttccagaccccttcctacacgcagccctaccaaggctcctccgtgacactgtccactgaggcttttctctacgtgggcaccatgttggatgggggcgacctgtcccgatttgcactgctcatgaccaactgctatgccacacccagtagcaatgccacggaccccctgaagtacttcatcatccaggacag

SEQ ID NO: 200 - DNA, 161 bp, Homo sapiens
atgcccacacactagagactcaactatccaagtggtggagaatggggagtcctcccagggccgattttccgtccagatgttccggtttgctggaaactatgacctagtctacctgcactgtgaagtctatctctgtgacaccatgaatgaaaagtgcaagc

SEQ ID NO: 201 - DNA, 325 bp, Homo sapiens
ggctcctgaaagtctggctgcctctgcttctctcggccaccttgaccctgacttttcagtgactgacagcggaaagccctgtgctccatggctgccatctcacctcctgctgggcagggggcatgatgcgggccagtgctccagccacagaaaagaaagttcatgctttgttcagcctgccttcttttctcccttttaatcctggctgtcgagaaacagcctgtgtctttaaatgctgctttttctcaaaatgggacttgtgacggtgtacctgaggcccccatctccttaaagagtgtggcaaaataatgatttttaaatctca

SEQ ID NO: 202 - DNA, 27 bp, Homo sapiens
agactaactctacctttctggcttcag

SEQ ID NO: 203 - DNA, 31 bp, Homo sapiens
gatggtgctctgaatgtcacagcaatgccac

SEQ ID NO: 204 - DNA, 45 bp, Homo sapiens
ctgcacggaggatgaggccgttacgacgtgcacctgtcaggaggg

SEQ ID NO: 205 - DNA, 16 bp, Homo sapiens
cttcaccggcgatggc

SEQ ID NO: 206 - DNA, 5 bp, Homo sapiens
ctgac

SEQ ID NO: 207 - DNA, 10 bp, Homo sapiens
ctgcgtggac

SEQ ID NO: 208 - DNA, 8 bp, Homo sapiens
ctggatga

SEQ ID NO: 209 - DNA, 11 bp, Homo sapiens
gtgcgccattc

SEQ ID NO: 210 - DNA, 31 bp, Homo sapiens
ctggagctcacaactgctccgccaacagcag

SEQ ID NO: 211 - DNA, 27 bp, Homo sapiens
ctgcgtaaacacgccaggctccttctc

SEQ ID NO: 212 - DNA, 11 bp, Homo sapiens
ctgcgtctgcc

SEQ ID NO: 213 - DNA, 6 bp, Homo sapiens
ccgaag

SEQ ID NO: 214 - DNA, 25 bp, Homo sapiens
gcttccgcctgtcgcccggtctcgg

SEQ ID NO: 215 - DNA, 18 bp, Homo sapiens
ctgcacagacgtggatga

SEQ ID NO: 216 - DNA, 10 bp, Homo sapiens
gtgcgctgag

SEQ ID NO: 217 - DNA, 13 bp, Homo sapiens
ctgggcttagcca

SEQ ID NO: 218 - DNA, 10 bp, Homo sapiens
ctgccacgcc

SEQ ID NO: 219 - DNA, 10 bp, Homo sapiens
ctggccacat

SEQ ID NO: 220 - DNA, 11 bp, Homo sapiens
gtgtcaatgtg

SEQ ID NO: 221 - DNA, 48 bp, Homo sapiens
gtgggcagctacttgtgcgtatgccccgcgggctaccggggggatgga

SEQ ID NO: 222 - DNA, 19 bp, Homo sapiens
tggcactgtgagtgctccc

SEQ ID NO: 223 - DNA, 7 bp, Homo sapiens
cgggctc

SEQ ID NO: 224 - DNA, 21 bp, Homo sapiens
ctgcgcggctggtaccgcttc

SEQ ID NO: 225 - DNA, 32 bp, Homo sapiens
gtgggccagggcggtgcgcgcatggccgagac

SEQ ID NO: 226 - DNA, 50 bp, Homo sapiens
ctgcgtgccagtcctgcgctgcaacacggccgcccccatgtggctcaatg

SEQ ID NO: 227 - DNA, 4 bp, Homo sapiens
gcac

SEQ ID NO: 228 - DNA, 25 bp, Homo sapiens
gcatccgtccagcgacgagggcatc

SEQ ID NO: 229 - DNA, 24 bp, Homo sapiens
gtgagccgcaaggcctgcgcgcac

SEQ ID NO: 230 - DNA, 99 bp, Homo sapiens
tggagcggccactgctgcctgtgggatgcgtccgtccaggtgaaggcctgtgccggcggctactacgtctacaacctgacagcgccccccgagtgtcac

SEQ ID NO: 231 - DNA, 16 bp, Homo sapiens
ctggcgtactgcacag

SEQ ID NO: 232 - DNA, 108 bp, Homo sapiens
accccagctccgtggaggggacgtgtgaggagtgcagtatagacgaggactgcaaatcgaataatggcagatggcactgccagtgcaaacaggacttcaacatcactg

SEQ ID NO: 233 - DNA, 23 bp, Homo sapiens
caccagaacacaagcccggcccg

SEQ ID NO: 234 - DNA, 92 bp, Homo sapiens
gtgagcagtccaccaatcagctcaggcctgatgaccagtccaccaatcagctcagtactccgggccaggcacaagcctcagatttctcatag

SEQ ID NO: 235 - DNA, 107 bp, Homo sapiens
atatctccctcctggagcacaggctggaatgtggggccaatgacatgaaggtgtcgctgggcaagtgccagctgaagagtctgggcttcgacaaggtcttcatgtac

SEQ ID NO: 236 - DNA, 55 bp, Homo sapiens
ctgagtgacagccggtgctcgggcttcaatgacagagacaaccgggactgggtgt

SEQ ID NO: 237 - DNA, 26 bp, Homo sapiens
ctgtagtgaccccagcccgggatggc

SEQ ID NO: 238 - DNA, 21 bp, Homo sapiens
ccctgtgggacagtgttgacg

SEQ ID NO: 239 - DNA, 2 bp, Homo sapiens
ct

SEQ ID NO: 240 - DNA, 82 bp, Homo sapiens
acctgctctgggaccagattccgaagtgggagtgtcatagatcaatcccgtgtcctgaacttgggtcccatcacacggaaag

SEQ ID NO: 241 - DNA, 39 bp, Homo sapiens
gtgtccaggccacagtctcaagggcttttagcagcttgg

SEQ ID NO: 242 - protein, 640 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

SEQ ID NO: 243 - protein, 640 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLDCEVYLCDTMNEKCKPTCSGTRFRSGSVIDQSRVLNLGPITRKGVQATVSRAFSSLGLLKVWLPLLLSATLTLTFQ

SEQ ID NO: 244 - protein, 611 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRPHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

SEQ ID NO: 245 - protein, 507 aa, Homo sapiens
MGQPSLTWMLMVVVASWFITTAATDTSEARWCSECHSNATCTEDEAVTTCTCQEGFTGDGLTCVDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

SEQ ID NO: 246 - protein, 376 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITAPEHKPGPVSSPPISSGLMTSPPISSVLRARHKPQISHRYLPPGAQAGMWGQ

SEQ ID NO: 247 - protein, 687 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSQTQERMLGTDEGAQLTWRKKSAHCKLLAGTSPGLAVSWSNVAPSSACALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

SEQ ID NO: 248 - protein, 558 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

SEQ ID NO: 249 - protein, 477 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVRCGQRGSLGPLDGSNPKPLNHELPCQLPPTGSWE

SEQ ID NO: 250 - DNA, 16 bp, Artificial Sequence
  Synthetic oligonucleotide
ggcccggtgagcagtc

SEQ ID NO: 251 - DNA, 24 bp, Artificial Sequence
  Synthetic oligonucleotide
ctatgagaaatctgaggcttgtgc

SEQ ID NO: 252 - DNA, 98 bp, Artificial Sequence
  Synthetic oligonucleotide
ggcccggtgagcagtccaccaatcagctcaggcctgatgaccagtccaccaatcagctcagtactccgggccaggcacaagcctcagatttctcatag

SEQ ID NO: 253 - DNA, 20 bp, Artificial Sequence
  Synthetic oligonucleotide
gtggccagagagggtcccta

SEQ ID NO: 254 - DNA, 19 bp, Artificial Sequence
  Synthetic oligonucleotide
cctgtgggtggcagttgac

SEQ ID NO: 255 - DNA, 87 bp, Artificial Sequence
  Synthetic oligonucleotide
tggccagagagggtccctagggcccctagatggttctaaccccaaaccccttaaccatgagcttccctgtcaactgccacccacagg

SEQ ID NO: 256 - DNA, 20 bp, Artificial Sequence
  Synthetic oligonucleotide
ctaccaaggctcctccgtga

SEQ ID NO: 257 - DNA, 21 bp, Artificial Sequence
  Synthetic oligonucleotide
tggtcatgagcagtgcaaatc

SEQ ID NO: 258 - DNA, 101 bp, Artificial Sequence
  Synthetic oligonucleotide
ctaccaaggctcctccgtgacactgtccactgaggcttttctctacgtgggcaccatgttggatgggggcgacctgtcccgatttgcactgctcatgacca

SEQ ID NO: 259 - DNA, 5724 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaaggtggcacctccatgtaagagaagcccaagacaaccccaaataatataatttcaaataggcagtgtcttttctgtgcttccaaggggaaggaggaaccttaccgataattgtttgtgaaagatccagggaagaaacaaaatttaaatgcactgctccccaacccctgcaataaccctccccatgtgtaggtacacacacacacacacacacacacacttaaaatggagaaaaatgaacagtttttttggtaagattcctgcactgtggctgcaggctttggtctttcctgggtctttcgagttgaggattgttgtaattagaaatggtaaattcaaagggaggaaattatgtaaagaaaaccaatctatgtctttcctccctaaaatggggaaaaagggaacatacaaatgaagaaaatataaatgtaaatatatttgaagatttaaaaagccctgaaaggttaacctttagtgcaaatggaaagttttaatttctgctcctgggtcttacacaaacaacttacatcatgaaaaactatttccattaatgccctttttattagagcagtcactgataacactagaatatcaaagactctaacagacaagccactaaatttaacgccaataacttgaacaaagtgaagagagccaaatatgagaaagaaaactttagtctttaaataacacttgcaaccatgtggttacatgtggttgcatgtaaaccacacaaaaggatggtcttgctcaaaaaaataatggtgagtaagtaacatggaggcatttggggaattcatgtgtgcaaatgcctggcatgcagagtatggcactacagggtaaccctacacacacagccacagagaagattcctcctgtgtaacaaccgtgaaagaagagggagattactgcttggttaagaaaggcgagatagtttttaaagctatagtaaaaattacagaacaaatagcaaatatgaagtaaaaatcaacactagataaaaaattaaagagaaatttgacttttgcaaagagaaaaggtattgcatgagactgaaagcagcagactgaataaaaaaagatagtttgaactatctttggcccataaaatcagtggccatgtttatcagtctgaatcatctttaaataagtccaataaagctttgagcaagtttacattaattatgagtcttagaaaagcctgcttagctttaaatttcttacaggcagggactatatattctgtatttctagctaagtacccaaatccaatacaatagtttgcttactgaatgtataacaacatattaattcaatgaagagaaacttttgaaacttttttgacataatttcagattcccatatacctttcacccagattccccaaatattaatgatgtgcgtagccacattggctttctcattcttttccctatctccctctcatgctcaccctctatatgtacacacacagacgtgtatagatacacatacaactatctttcctaaaaacaagaatattcttgtacatgaccacaatacaatgatcgaaattagaaaagcaacaatgatttaaaaactagagatcttcaaatttcgctaataatcctaatcatgttctttacagaaaaagaaaatcccggatcatgtactgcattgttgccatgtcttttaagtctcctttactctgaataattccttaatacagggcactttgtagaatgtccttcaatttgagtttgtctaatgtttccttatgattagatttaggttatgcacttttggcaagactatcatggcatgatgttgtgtttttcttagtacaccatatcaggaggcacaagatattgatcagttctattactggttatgttaacttcaagctttggttaagatggtgtctgccaggtgtctccattgtaaagttattcattttccctttacaattaataaagatcttgtagggaagaaagtaatctttctttaaataaagcagcaatagaatgagagatttttaaacatataaaaagcagaaaaatataaatgaatcaggagtaaagtaacattaaaaattggaatacatttaaagaaccatctactaatttctaaccatatattatttttaataatggtcttaaaatttctttttctatagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcata

SEQ ID NO: 260 - DNA, 3635 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaagtaattctccagactctgattcccaatttcagcagtaattttaggtttccttttcagagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcata

SEQ ID NO: 261 - DNA, 4670 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaaggtgaatatccagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcatacaactttgtacatttgtgggggaaaactattaattttttgcaaatggaaagatcaacagactatataatgatacatgactgacacttgtacactaggtaataaaactgattcatacagtctaatgatatcaccgctgttagggttttataaaactgcatttaaaaaaagatctatgaccagatattctcctgggtgctcctcaaaggaacactattaaggttcattgaaatgttttcaatcattgccttcccattgatccttctaacatgctgttgacatcacacctaatattcagagggaatgggcaaggtatgagggaaggaaataaaaaataaaataaataaaatagaatgacacaaatttgagttttgtgaacccctgaacagatggtcttaaggacgttatctggaactggagaaaagcagagttgagagacaattctatagattaaatcctggtaaggacaaacattgccattagaagaaaagcttcaaaatagacctgtggcagatgtcacatgagtagaatttctgcccagccttaactgcattcagaggataatatcaatgaactaaacttgaactaaaaattttttaaacaaaaagttataaatgaagacacatggttgtgaatacaatgatgtatttctttattttcacatacactctagctaaaagagcaagagtacacatcaacaaaaatggaaacaaggctttggctgaaaaaaacatgcatttgacaaatcatgttaatagctagacaagaagaaagttagctttgtaaacttctacttcatttgattcagagaaacagagcatgagttttcttaaaagtaacaagaaaaggaacaaaaaaaatgaggtttgaaatcttttaccatggcaaaacattaacatctttctcaaaaacatagagaaatctggaaaaatcaagaagataaaattctggaccagttagtgacattctttcaagcatacttgtaaaatgtttccttaaagtgttcttgggatgaaaatgattgtcatgtctccaacaacagtgaactgatgttgttccttggaataaaagtcaatccccacctta

SEQ ID NO: 262 - DNA, 3569 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcata

SEQ ID NO: 263 - DNA, 3513 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcata

SEQ ID NO: 264 - DNA, 3321 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaaggtaagagctatccatggcaattactcttgctctgtttgaaaatgttttaatac

SEQ ID NO: 265 - DNA, 3361 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcata

SEQ ID NO: 266 - DNA, 3326 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacgggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcata

SEQ ID NO: 267 - DNA, 3187 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgaggctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcata

SEQ ID NO: 268 - DNA, 2939 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtgaaactctcacttacgtatggaaaatcccagaaagatctggagctggaacagaggattctgcttgtattccatgggcttattattcaactgtggatcaagttaaggacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccatgtgaccgaccacattcatgctggaatggaaaccacttacaccgttctacaaaatgaagacaccaaatctggctgaatgaaataaattggtgataagtggaaaaaagagaaaaaccaatgattcataacaatgtatgtgaaagtgtaaaatagaatgttactttggaatgactataaacattaaaagaagactggaagcata

SEQ ID NO: 269 - DNA, 3996 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagaggggacttttaatgttgaatgccttacaactgatcattacacaggcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaatgtttcaaatgcatttttagataagggagagttttacataggctcaaagtacaagaaagttgtgtatcggcagtatactgatagcacattccgtgttccagtggagagaaaagctgaagaagaacatctgggaattctaggtccacaacttcatgcagatgttggagacaaagtcaaaattatctttaaaaacatggccacaaggccctactcaatacatgcccatggggtacaaacagagagttctacagttactccaacattaccaggtactcacgggggcgggggtgggggtggtgcattttaacaggctgtacagcctttccacagattactctcatcatagagagtctggaccaagaaagtgaaaagggaggtagttgtgaagaggggatgcaaggaggtgtctgtgtgtatgcaagtacatgcacatgcttgtactaaaaaaaaaagtccacaggagagcttaagagaaaaacctgaaggtagtagacatctatgaatcctcaagcgtgaatcagagagatgctgtctcctcgctcagctagaacacagctcaaatggcatctaccacaccatatgtggctggatgttcccatgcctgtctctagcacatgtggttctaagggccagaggctgccacttggaactctaaattccccctatctagtacagtatctggcatatattaggagatcatcaaatgtttctttaataaacatagagtaaaatcttgaacagattatatacataggtgagcattttctttaataagaaccatatttttcttctattagttgtaaagttggcttccaggaaaatactatttctcaataacttagcagggtgtagcaagggtgctattttaggtagagaggtcaggagggcttccttgaggaggtggcattgctgaaaacacctaaagtttgagaagtaatgaaattcaggtaattgtgcacgaattgattctaggccccgaagacaggagcaatgcctggtgtgtgtgagcagcagcaaggcagccagaggctaaagaaaagcacacaagggacagaatgctagagatgagatccagggagagcaggggccagatggagtagggaggacctcagactcgagatttaagactgatgttctgatggacaccttttaaggggctgttggtgatgtgtggttaagtgaccctaagggggcaaggacagaatctggggtcagttaaggaccactgcaataatctgggtaaaatcatcatcatcatcatcatcatcgagatttggaatatggtggaagcggctgaagtggtgagatgttatcatattctggttccattttaaaggtggtactaaaaggacctgctggaaaatgggcactagggagtgttagggaagagaggagtcaagcatgaccccaaggttttgggcactgaccactggtggaagagcaggttgtaaaggggtgtgg

SEQ ID NO: 270 - DNA, 2247 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacagaaagatgtagacaaggaattctatttgtttcctacagtatttgatgagaatgagagtttactcctggaagataatattagaatgtttacaactgcacctgatcaggtggataaggaagatgaagactttcaggaatctaataaaatgcactgtaagtactgcatcatccaccaatctaccaagttattttagtactgcatatgtaatgcttttaactaaacaaaatgtgaagagctttgctcattgattattttttcaataaacgattacaatagtgtcctgaa

SEQ ID NO: 271 - DNA, 1779 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacacggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgcctctaggccctacacctttcattcacatggaataacttactataaggaacatgagggggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcatcggacctttaataatctgtaaaaaagattctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtattctgtgaatggatacacttttggaagtctcccaggactctccatgtgtgctgaagacagagtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaaccctggagaatggatgctcagctgtcagaatctaaaccatctgaaagccggtttgcaagcctttttccaggtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaagtgactcagcggtgttttttgaacaaggtaccacaagaattggaggctcttataaaaagctggtttatcgtgagtacacagatgcctccttcacaaatcgaaaggagagaggccctgaagaagagcatcttggcatcctgggtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaagtgagtatacagcactgtgtaacaaatc

SEQ ID NO: 272 - DNA, 396 bp, Homo sapiens
ccctaatgcctccaacaataactgttgactttttattttcagtcagagaagcctggcaaccaagaactgtttttttggtggtttacgagaacttaactgaattggaaaatatttgctttaatgaaacaatttactcttgtgcaacactaaattgtgtcaatcaagcaaataaggaagaaagtcttatttataaaattgcctgctcctgattttacttcatttcttctcaggctccaagaaggggaaaaaaatgaagattttgatacttggtatttttctgtttttatgtagtaccccagcctgggcgaaagaaaagcattattacattggaattattgaaacgacttgggattatgcctctgaccatggggaaaagaaacttatttctgttgacac

SEQ ID NO: 273 - DNA, 192 bp, Homo sapiens
ggaacattccaatatctatcttcaaaatggcccagatagaattgggagactatataagaaggccctttatcttcagtacacagatgaaacctttaggacaactatagaaaaaccggtctggcttgggtttttaggccctattatcaaagctgaaactggagataaagtttatgtacacttaaaaaaccttgc

SEQ ID NO: 274 - DNA, 187 bp, Homo sapiens
gggccatctaccctgataacaccacagattttcaaagagcagatgacaaagtatatccaggagagcagtatacatacatgttgcttgccactgaagaacaaagtcctggggaaggagatggcaattgtgtgactaggatttaccattcccacattgatgctccaaaagatattgcctcaggactcat

SEQ ID NO: 275 - DNA, 174 bp, Homo sapiens
attctctagataaagaaaaagaaaaacatattgaccgagaatttgtggtgatgttttctgtggtggatgaaaatttcagctggtacctagaagacaacattaaaacctactgctcagaaccagagaaagttgacaaagacaacgaagacttccaggagagtaacagaatgtatt

SEQ ID NO: 276 - DNA, 152 bp, Homo sapiens
agtaaaatggtacctttttggtatgggtaatgaagttgatgtgcacgcagctttctttcacgggcaagcactgactaacaagaactaccgtattgacacaatcaacctctttcctgctaccctgtttgatgcttatatggtggcccagaacc

SEQ ID NO: 277 - DNA, 149 bp, Homo sapiens
gtccaggagtgtaacaagtcttcatcaaaggataatatccgtgggaagcatgttagacactactacattgccgctgaggaaatcatctggaactatgctccctctggtatagacatcttcactaaagaaaacttaacagcacctggaag

SEQ ID NO: 278 - DNA, 140 bp, Homo sapiens
tgactcagcggtgttttttgaacaaggtaccacaagaattggaggctcttataaaaagctggtttatcgtgagtacacagatgcctccttcacaaatcgaaaggagagaggccctgaagaagagcatcttggcatcctgg

SEQ ID NO: 279 - DNA, 153 bp, Homo sapiens
gtcctgtcatttgggcagaggtgggagacaccatcagagtaaccttccataacaaaggagcatatcccctcagtattgagccgattggggtgagattcaataagaacaacgagggcacatactattccccaaattacaacccccagagcagaa

SEQ ID NO: 280 - DNA, 212 bp, Homo sapiens
gtgtgcctccttcagcctcccatgtggcacccacagaaacattcacctatgaatggactgtccccaaagaagtaggacccactaatgcagatcctgtgtgtctagctaagatgtattattctgctgtggatcccactaaagatatattcactgggcttattgggccaatgaaaatatgcaagaaaggaagtttacatgcaaatgggagacag

SEQ ID NO: 281 - DNA, 151 bp, Homo sapiens
aaagatgtagacaaggaattctatttgtttcctacagtatttgatgagaatgagagtttactcctggaagataatattagaatgtttacaactgcacctgatcaggtggataaggaagatgaagactttcaggaatctaataaaatgcact

SEQ ID NO: 282 - DNA, 133 bp, Homo sapiens
gtaagtactgcatcatccaccaatctaccaagttattttagtactgcatatgtaatgcttttaactaaacaaaatgtgaagagctttgctcattgattattttttcaataaacgattacaatagtgtcctgaa

SEQ ID NO: 283 - DNA, 213 bp, Homo sapiens
ccatgaatggattcatgtatgggaatcagccgggtctcactatgtgcaaaggagattcggtcgtgtggtacttattcagcgccggaaatgaggccgatgtacatggaatatacttttcaggaaacacatatctgtggagaggagaacggagagacacagcaaacctcttccctcaaacaagtcttacgctccacatgtggcctgacacagagg

SEQ ID NO: 284 - DNA, 166 bp, Homo sapiens
gcggcatgaagcaaaaatatactgtgaaccaatgcaggcggcagtctgaggattccaccttctacctgggagagaggacatactatatcgcagcagtggaggtggaatgggattattccccacaaagggagtgggaaaaggagctgcatcatttacaagagcagaa

SEQ ID NO: 285 - DNA, 140 bp, Homo sapiens
tgtttcaaatgcatttttagataagggagagttttacataggctcaaagtacaagaaagttgtgtatcggcagtatactgatagcacattccgtgttccagtggagagaaaagctgaagaagaacatctgggaattctag

SEQ ID NO: 286 - DNA, 1192 bp, Homo sapiens
gtactcacgggggcgggggtgggggtggtgcattttaacaggctgtacagcctttccacagattactctcatcatagagagtctggaccaagaaagtgaaaagggaggtagttgtgaagaggggatgcaaggaggtgtctgtgtgtatgcaagtacatgcacatgcttgtactaaaaaaaaaagtccacaggagagcttaagagaaaaacctgaaggtagtagacatctatgaatcctcaagcgtgaatcagagagatgctgtctcctcgctcagctagaacacagctcaaatggcatctaccacaccatatgtggctggatgttcccatgcctgtctctagcacatgtggttctaagggccagaggctgccacttggaactctaaattccccctatctagtacagtatctggcatatattaggagatcatcaaatgtttctttaataaacatagagtaaaatcttgaacagattatatacataggtgagcattttctttaataagaaccatatttttcttctattagttgtaaagttggcttccaggaaaatactatttctcaataacttagcagggtgtagcaagggtgctattttaggtagagaggtcaggagggcttccttgaggaggtggcattgctgaaaacacctaaagtttgagaagtaatgaaattcaggtaattgtgcacgaattgattctaggccccgaagacaggagcaatgcctggtgtgtgtgagcagcagcaaggcagccagaggctaaagaaaagcacacaagggacagaatgctagagatgagatccagggagagcaggggccagatggagtagggaggacctcagactcgagatttaagactgatgttctgatggacaccttttaaggggctgttggtgatgtgtggttaagtgaccctaagggggcaaggacagaatctggggtcagttaaggaccactgcaataatctgggtaaaatcatcatcatcatcatcatcatcgagatttggaatatggtggaagcggctgaagtggtgagatgttatcatattctggttccattttaaaggtggtactaaaaggacctgctggaaaatgggcactagggagtgttagggaagagaggagtcaagcatgaccccaaggttttgggcactgaccactggtggaagagcaggttgtaaaggggtgtgg

SEQ ID NO: 287 - DNA, 217 bp, Homo sapiens
gacctctacagtggattaattggccccctgattgtttgtcgaagaccttacttgaaagtattcaatcccagaaggaaactggaatttgcccttctgtttctagtttttgatgagaatgaatcttggtacttagatgacaacatcaaaacatactctgatcaccccgagaaagtaaacaaagatgatgaggaattcatagaaagcaataaaatgcatg

SEQ ID NO: 288 - DNA, 140 bp, Homo sapiens
ctattaatggaagaatgtttggaaacctacaaggcctcacaatgcacgtgggagatgaagtcaactggtatctgatgggaatgggcaatgaaatagacttacacactgtacattttcacggccatagcttccaatacaag

SEQ ID NO: 289 - DNA, 2150 bp, Homo sapiens
gtggcacctccatgtaagagaagcccaagacaaccccaaataatataatttcaaataggcagtgtcttttctgtgcttccaaggggaaggaggaaccttaccgataattgtttgtgaaagatccagggaagaaacaaaatttaaatgcactgctccccaacccctgcaataaccctccccatgtgtaggtacacacacacacacacacacacacacttaaaatggagaaaaatgaacagtttttttggtaagattcctgcactgtggctgcaggctttggtctttcctgggtctttcgagttgaggattgttgtaattagaaatggtaaattcaaagggaggaaattatgtaaagaaaaccaatctatgtctttcctccctaaaatggggaaaaagggaacatacaaatgaagaaaatataaatgtaaatatatttgaagatttaaaaagccctgaaaggttaacctttagtgcaaatggaaagttttaatttctgctcctgggtcttacacaaacaacttacatcatgaaaaactatttccattaatgccctttttattagagcagtcactgataacactagaatatcaaagactctaacagacaagccactaaatttaacgccaataacttgaacaaagtgaagagagccaaatatgagaaagaaaactttagtctttaaataacacttgcaaccatgtggttacatgtggttgcatgtaaaccacacaaaaggatggtcttgctcaaaaaaataatggtgagtaagtaacatggaggcatttggggaattcatgtgtgcaaatgcctggcatgcagagtatggcactacagggtaaccctacacacacagccacagagaagattcctcctgtgtaacaaccgtgaaagaagagggagattactgcttggttaagaaaggcgagatagtttttaaagctatagtaaaaattacagaacaaatagcaaatatgaagtaaaaatcaacactagataaaaaattaaagagaaatttgacttttgcaaagagaaaaggtattgcatgagactgaaagcagcagactgaataaaaaaagatagtttgaactatctttggcccataaaatcagtggccatgtttatcagtctgaatcatctttaaataagtccaataaagctttgagcaagtttacattaattatgagtcttagaaaagcctgcttagctttaaatttcttacaggcagggactatatattctgtatttctagctaagtacccaaatccaatacaatagtttgcttactgaatgtataacaacatattaattcaatgaagagaaacttttgaaacttttttgacataatttcagattcccatatacctttcacccagattccccaaatattaatgatgtgcgtagccacattggctttctcattcttttccctatctccctctcatgctcaccctctatatgtacacacacagacgtgtatagatacacatacaactatctttcctaaaaacaagaatattcttgtacatgaccacaatacaatgatcgaaattagaaaagcaacaatgatttaaaaactagagatcttcaaatttcgctaataatcctaatcatgttctttacagaaaaagaaaatcccggatcatgtactgcattgttgccatgtcttttaagtctcctttactctgaataattccttaatacagggcactttgtagaatgtccttcaatttgagtttgtctaatgtttccttatgattagatttaggttatgcacttttggcaagactatcatggcatgatgttgtgtttttcttagtacaccatatcaggaggcacaagatattgatcagttctattactggttatgttaacttcaagctttggttaagatggtgtctgccaggtgtctccattgtaaagttattcattttccctttacaattaataaagatcttgtagggaagaaagtaatctttctttaaataaagcagcaatagaatgagagatttttaaacatataaaaagcagaaaaatataaatgaatcaggagtaaagtaacattaaaaattggaatacatttaaagaaccatctactaatttctaaccatatattatttttaataatggtcttaaaatttctttttctatag

SEQ ID NO: 290 - DNA, 1403 bp, Homo sapiens
acaccaaatctggctgaatgaaataaattggtgataagtggaaaaaagagaaaaaccaatgattcataacaatgtatgtgaaagtgtaaaatagaatgttactttggaatgactataaacattaaaagaagactggaagcatacaactttgtacatttgtgggggaaaactattaattttttgcaaatggaaagatcaacagactatataatgatacatgactgacacttgtacactaggtaataaaactgattcatacagtctaatgatatcaccgctgttagggttttataaaactgcatttaaaaaaagatctatgaccagatattctcctgggtgctcctcaaaggaacactattaaggttcattgaaatgttttcaatcattgccttcccattgatccttctaacatgctgttgacatcacacctaatattcagagggaatgggcaaggtatgagggaaggaaataaaaaataaaataaataaaatagaatgacacaaatttgagttttgtgaacccctgaacagatggtcttaaggacgttatctggaactggagaaaagcagagttgagagacaattctatagattaaatcctggtaaggacaaacattgccattagaagaaaagcttcaaaatagacctgtggcagatgtcacatgagtagaatttctgcccagccttaactgcattcagaggataatatcaatgaactaaacttgaactaaaaattttttaaacaaaaagttataaatgaagacacatggttgtgaatacaatgatgtatttctttattttcacatacactctagctaaaagagcaagagtacacatcaacaaaaatggaaacaaggctttggctgaaaaaaacatgcatttgacaaatcatgttaatagctagacaagaagaaagttagctttgtaaacttctacttcatttgattcagagaaacagagcatgagttttcttaaaagtaacaagaaaaggaacaaaaaaaatgaggtttgaaatcttttaccatggcaaaacattaacatctttctcaaaaacatagagaaatctggaaaaatcaagaagataaaattctggaccagttagtgacattctttcaagcatacttgtaaaatgtttccttaaagtgttcttgggatgaaaatgattgtcatgtctccaacaacagtgaactgatgttgttccttggaataaaagtcaatccccaccttaaaaaatgtatggcttctttgaggaattcttatgtcttaaagactttttacattctagacaattaaattgattgaggtcataaattaagaagtgaatagttaccactacacggtaaggtaagcagcctgaaagcatttgtatcatatatgtgtgtgtgtgtgtgtgtgtgtgtgtgt

SEQ ID NO: 291 - DNA, 56 bp, Homo sapiens
ctctaggccctacacctttcattcacatggaataacttactataaggaacatgagg

SEQ ID NO: 292 - DNA, 26 bp, Homo sapiens
cggacctttaataatctgtaaaaaag

SEQ ID NO: 293 - DNA, 58 bp, Homo sapiens
ctgtgaatggatacacttttggaagtctcccaggactctccatgtgtgctgaagacag

SEQ ID NO: 294 - DNA, 45 bp, Homo sapiens
ctggagaatggatgctcagctgtcagaatctaaaccatctgaaag

SEQ ID NO: 295 - DNA, 23 bp, Homo sapiens
ccggtttgcaagcctttttccag

SEQ ID NO: 296 - DNA, 28 bp, Homo sapiens
gtgagtatacagcactgtgtaacaaatc

SEQ ID NO: 297 - DNA, 42 bp, Homo sapiens
ggacttttaatgttgaatgccttacaactgatcattacacag

SEQ ID NO: 298 - DNA, 61 bp, Homo sapiens
gtaattctccagactctgattcccaatttcagcagtaattttaggtttccttttcagagaa

SEQ ID NO: 299 - DNA, 57 bp, Homo sapiens
gtccacaacttcatgcagatgttggagacaaagtcaaaattatctttaaaaacatgg

SEQ ID NO: 300 - DNA, 72 bp, Homo sapiens
ccacaaggccctactcaatacatgcccatggggtacaaacagagagttctacagttactccaacattaccag

SEQ ID NO: 301 - DNA, 77 bp, Homo sapiens
gtgaaactctcacttacgtatggaaaatcccagaaagatctggagctggaacagaggattctgcttgtattccatgg

SEQ ID NO: 302 - DNA, 30 bp, Homo sapiens
gcttattattcaactgtggatcaagttaag

SEQ ID NO: 303 - DNA, 53 bp, Homo sapiens
gtaagagctatccatggcaattactcttgctctgtttgaaaatgttttaatac

SEQ ID NO: 304 - DNA, 5 bp, Homo sapiens
cacag

SEQ ID NO: 305 - DNA, 56 bp, Homo sapiens
gggagtttatagttctgatgtctttgacattttccctggaacataccaaaccctag

SEQ ID NO: 306 - DNA, 102 bp, Homo sapiens
aaatgtttccaagaacacctggaatttggttactccactgccatgtgaccgaccacattcatgctggaatggaaaccacttacaccgttctacaaaatgaag

SEQ ID NO: 307 - DNA, 12 bp, Homo sapiens
gtgaatatccag

SEQ ID NO: 308 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 309 - protein, 215 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKKKKKKKKK

SEQ ID NO: 310 - protein, 1065 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEGGTSM

SEQ ID NO: 311 - protein, 762 aa, Homo sapiens [sequence omitted]

SEQ ID NO: 312 - protein, 1069 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEGEYPDTKSG

SEQ ID NO: 313 - protein, 1009 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKGSL

SEQ ID NO: 314 - protein, 1021 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKKCFQEHLEFGYSTAM

SEQ ID NO: 315 - protein, 1018 aa, Homo sapiens [sequence omitted]

SEQ ID NO: 316 - protein, 994 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHWTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 317 - protein, 936 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEAVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 318 - protein, 863 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGTHGGGGGGGAF

SEQ ID NO: 319 - protein, 634 aa, Homo sapiens [sequence omitted]

SEQ ID NO: 320 - protein, 509 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSEYTALCNK

SEQ ID NO: 321 - protein, 53 aa, Homo sapiens
MKILILGIFLFLCSTPAWAKEKHYYIGIIETTWDYASDHGEKKLISVDTGHLP

SEQ ID NO: 322 - protein, 65 aa, Homo sapiens
MKILILGIFLFLCSTPAWAKEKHYYIGIIETTWDYASDHGEKKLISVDTCEWIHFWKSPRTLHVC

SEQ ID NO: 323 - DNA, 26 bp, Artificial Sequence
  Synthetic oligonucleotide
ggaattattgaaacgacttgggatta

SEQ ID NO: 324 - DNA, 25 bp, Artificial Sequence
  Synthetic oligonucleotide
aaagtgtatccattcacaggtgtca

SEQ ID NO: 325 - DNA, 87 bp, Artificial Sequence
  Synthetic oligonucleotide
ggaattattgaaacgacttgggattatgcctctgaccatggggaaaagaaacttatttctgttgacacctgtgaatggatacacttt

SEQ ID NO: 326 - DNA, 23 bp, Artificial Sequence
  Synthetic oligonucleotide
tcaaaatggcccagatagaattg

SEQ ID NO: 327 - DNA, 25 bp, Artificial Sequence
  Synthetic oligonucleotide
gccagaccggtttttctatagttgt

SEQ ID NO: 328 - DNA, 101 bp, Artificial Sequence
  Synthetic oligonucleotide
tcaaaatggcccagatagaattgggagactatataagaaggccctttatcttcagtacacagatgaaacctttaggacaactatagaaaaaccggtctggc

SEQ ID NO: 329 - DNA, 3292 bp, Homo sapiens
attgagctaagaggagctgacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggtcagctttggggtggggaccagaggtggcataggagatgttccctgttggagccacagggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcca

SEQ ID NO: 330 - DNA, 4096 bp, Homo sapiens
attgagctaagaggagctgacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaaggtggccctgcttcccgcccactgcctgggttgggagaggggagattgtttctggaaggggccatctcccttctgtgcccaggtttcctttcccagacgctgaggaaggctggccctgcctcccttgtgtccacagcactggctgctcagctatgacctgtctccttcctgcgcctgggctcagccagctggcagccagggccgtccatttgctcactgcctctggcaggccaagggagagagggttgttcctccttggctgacaggagtcctgttggtggaatcactggctctttgttaggttgggagagggttctgggaggagaccttctcagtgccccccaagcctccatgctcagtcctgtttccgccccactgagagggcagccccaacccccatcacacagaaagagagagcctgggggtctggtgggtgccgtgggacagggctgggagtgggcagggtggtcgaggcactgctgagtagggaaggaggaggagagaaaggagagagcgcctgagagagggagagacagacaggagaatgtcagggccagagggagagcaggcacagcagagagaaggggagagagacgggcgacgctttagtgaggagggatccgaggcctggcggtgcccagacccaggggttgcccaggttcccagttcagtgttctgctcattaaccctgcacctcagaggctgttgctgatggcgccctaggcagcgatccctttcgggcctccggaggcctctctgcaggttgagggtaccagaggtcctgagggcagggggagatccagttctgcctgggcaccttccttgccctcggtgagccagtctagcctctctctgtgcctcagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcca

SEQ ID NO: 331 - DNA, 3200 bp, Homo sapiens
attgagctaagaggagctgacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcca

SEQ ID NO: 332 - DNA, 3729 bp, Homo sapiens
attgagctaagaggagctgacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtaagcaggacctaggccaggagcgagtgggctgacagctaaaggtggggtagggatcaccttggctctagggagctagggtggggagcagtctgctttcctcccatcttggagatctggtctgactctctagcctcagttcctctgactctgcttccatatctgataataaacagtggggtggggatggccctcagagggaactggctacttcctatgtgttgtggctcgctgtgacccaggaagccagggcctgggagcatcccaatcttgcaagtctgaatggcataatctcctgagacctatatgtccctggggtcatgtagtgaccggtgaggacctgctcactggggccaccaccttggcaccaggcactgcttgtgcccagccctcttctcgaatcctcctgaccctacctggacttgtccctgactccaatctgagctctgatactgagccagatacttcccctgacctggctcctctggtccccaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcca

SEQ ID NO: 333 - DNA, 3316 bp, Homo sapiens
attgagctaagaggagctgacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaaggtggggcacgaggcactgcccagctctgggcaaggatgtcccaggcctttcagagaagcagcaggcagcagggagcttgaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcaggggacacccgttccagtgagatgcccgagctcacctccatgcacaccctcttacttcgggagcacaaccggctggccacagagctcaagagcctgaaccctaggtgggatggggagaggctctaccaggaagcccggaagatcgtgggggccatggtccagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcca

SEQ ID NO: 334 - DNA, 3073 bp, Homo sapiens
attgagctaagaggagctgacaatatcaggtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcagatgcatggtggacttaggaccttgctgggctgggggtctcactgcagagatgaagctgcttctggccctagcagggctcctggccattctggccacgccccagccctctgaaggtgctgctccagctgtcctgggggaggtggacacctcgttggtgctgagctccatggaggaggccaagcagctggtggacaaggcctacaaggagcggcgggaaagcatcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactgatgtgctgacgcccgcccagctgaatgtgttgtccaagtcaagcggctgcgcctaccaggacgtgggggtgacttgcccggagcaggacaaataccgcaccatcaccgggatgtgcaacaacagacgcagccccacgctgggggcctccaaccgtgcctttgtgcgctggctgccggcggagtatgaggacggcttctctcttccctacggctggacgcccggggtcaagcgcaacggcttcccggtggctctggctcgcgcggtctccaacgagatcgtgcgcttccccactgatcagctgactccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaagatcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcagatcatcacttaccgggactacctgcccctggtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaaggtggcattgaccccatcctccggggcctcatggccacccctgccaagctgaatcgtcagaaccaaattgcagtggatgagatccgggagcgattgtttgagcaggtcatgaggattgggctggacctgcctgctctgaacatgcagcgcagcagggaccacggcctcccaggatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcggtacccagttcaggaagctccgggatggtgatcggttttggtgggagaacgagggtgtgttcagcatgcagcagcgacaggccctggcccagatctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctggccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgcctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctcca

SEQ ID NO: 335 - DNA, 195 bp, Homo sapiens
gtgagctgtggaggtggggtccttggaagctggatgacagcagctggcaaggggataagagagcagtgagcccctccctcaaggaggtctggctttatccatagacagggccctctgaggtggggctgaggtacaaagggggattgagcagcccaggagaagagagatgggggttcccttcttctcttctctcag

SEQ ID NO: 336 - DNA, 125 bp, Homo sapiens
atgcatggtggacttaggaccttgctgggctgggggtctcactgcagagatgaagctgcttctggccctagcagggctcctggccattctggccacgccccagccctctgaaggtgctgctccag

SEQ ID NO: 337 - DNA, 176 bp, Homo sapiens
catcaagcagcggcttcgcagcggctcagccagccccatggaactcctatcctacttcaagcagccggtggcagccaccaggacggcggtgagggccgctgactacctgcacgtggctctagacctgctggagaggaagctgcggtccctgtggcgaaggccattcaatgtcactg

SEQ ID NO: 338 - DNA, 124 bp, Homo sapiens
atgtgctgacgcccgcccagctgaatgtgttgtccaagtcaagcggctgcgcctaccaggacgtgggggtgacttgcccggagcaggacaaataccgcaccatcaccgggatgtgcaacaacag

SEQ ID NO: 339 - DNA, 130 bp, Homo sapiens
acgcagccccacgctgggggcctccaaccgtgcctttgtgcgctggctgccggcggagtatgaggacggcttctctcttccctacggctggacgcccggggtcaagcgcaacggcttcccggtggctctg

SEQ ID NO: 340 - DNA, 156 bp, Homo sapiens
ccggaccaggagcgctcactcatgttcatgcaatggggccagctgttggaccacgacctcgacttcacccctgagccggccgcccgggcctccttcgtcactggcgtcaactgcgagaccagctgcgttcagcagccgccctgcttcccgctcaag

SEQ ID NO: 341 - DNA, 862 bp, Homo sapiens
gtggccctgcttcccgcccactgcctgggttgggagaggggagattgtttctggaaggggccatctcccttctgtgcccaggtttcctttcccagacgctgaggaaggctggccctgcctcccttgtgtccacagcactggctgctcagctatgacctgtctccttcctgcgcctgggctcagccagctggcagccagggccgtccatttgctcactgcctctggcaggccaagggagagagggttgttcctccttggctgacaggagtcctgttggtggaatcactggctctttgttaggttgggagagggttctgggaggagaccttctcagtgccccccaagcctccatgctcagtcctgtttccgccccactgagagggcagccccaacccccatcacacagaaagagagagcctgggggtctggtgggtgccgtgggacagggctgggagtgggcagggtggtcgaggcactgctgagtagggaaggaggaggagagaaaggagagagcgcctgagagagggagagacagacaggagaatgtcagggccagagggagagcaggcacagcagagagaaggggagagagacgggcgacgctttagtgaggagggatccgaggcctggcggtgcccagacccaggggttgcccaggttcccagttcagtgttctgctcattaaccctgcacctcagaggctgttgctgatggcgccctaggcagcgatccctttcgggcctccggaggcctctctgcaggttgagggtaccagaggtcctgagggcagggggagatccagttctgcctgggcaccttccttgccctcggtgagccagtctagcctctctctgtgcctcag

SEQ ID NO: 342 - DNA, 319 bp, Homo sapiens
atcccgcccaatgacccccgcatcaagaaccaagccgactgcatcccgttcttccgctcctgcccggcttgccccgggagcaacatcaccatccgcaaccagatcaacgcgctcacttccttcgtggacgccagcatggtgtacggcagcgaggagcccctggccaggaacctgcgcaacatgtccaaccagctggggctgctggccgtcaaccagcgcttccaagacaacggccgggccctgctgccctttgacaacctgcacgatgacccctgtctcctcaccaaccgctcagcgcgcatcccctgcttcctggcag

SEQ ID NO: 343 - DNA, 226 bp, Homo sapiens
gtgctggggccaacggccatgaggaagtacctgcccacgtaccgttcctacaatgactcagtggacccacgcatcgccaacgtcttcaccaatgccttccgctacggccacaccctcatccaacccttcatgttccgcctggacaatcggtaccagcccatggaacccaacccccgtgtccccctcagcagggtcttttttgcctcctggagggtcgtgctggaag

SEQ ID NO: 344 - DNA, 495 bp, Homo sapiens
gtaagcaggacctaggccaggagcgagtgggctgacagctaaaggtggggtagggatcaccttggctctagggagctagggtggggagcagtctgctttcctcccatcttggagatctggtctgactctctagcctcagttcctctgactctgcttccatatctgataataaacagtggggtggggatggccctcagagggaactggctacttcctatgtgttgtggctcgctgtgacccaggaagccagggcctgggagcatcccaatcttgcaagtctgaatggcataatctcctgagacctatatgtccctggggtcatgtagtgaccggtgaggacctgctcactggggccaccaccttggcaccaggcactgcttgtgcccagccctcttctcgaatcctcctgaccctacctggacttgtccctgactccaatctgagctctgatactgagccagatacttcccctgacctggctcctctggtccccag

SEQ ID NO: 345 - DNA, 204 bp, Homo sapiens
gatacaatgcctggaggcgcttctgtgggctcccgcagcctgaaactgtgggccagctgggcacggtgctgaggaacctgaaattggcgaggaaactgatggagcagtatggcacgcccaacaacatcgacatctggatgggcggcgtgtccgagcctctgaagcgcaaaggccgcgtgggcccactcctcgcctgcatcatcg

SEQ ID NO: 346 - DNA, 194 bp, Homo sapiens
atctcattgccccggatcatctgcgacaacacaggcatcaccaccgtgtctaagaacaacatcttcatgtccaactcatatccccgggactttgtcaactgcagtacacttcctgcattgaacctggcttcctggagggaagcctcctagaggccaggtaagggggtgcagcagtgaggggtatatctgggctg

SEQ ID NO: 347 - DNA, 282 bp, Homo sapiens
gccagttggaaccacggagatctccttgccctagatgagcccagccctgttctgggtgcagctgagaaaatgagtgactagacgttcatttgtgtgctcatgtatgtgcgaagtatataaattggcttttcatgcgtgtgtgttgtctgaacatggggagtgtttcatgggttatgtgtatgtgccatttatgtgagtgtgtgtttgtgctgatgagaatactgagtatgtggaaggcagcagagcggactggtgaggagcacagctcaggaactagactgc

SEQ ID NO: 348 - DNA, 596 bp, Homo sapiens
ctgggttccaatcctggctctgtggcttgctagctatgtgaccttgagcaaattaccctccttaaacaagagttttcttccttgtaaattacatctgtcatggtttcttggagggcccacttgtatcctctggttcttcatttattgagcacctactacatgcaaggcactgtactaggcgtgagaagcatatagaggcaagaaagagataccaagatgccatctgtgtcctggttagcagagctggaccagtggtgccttggagggataagccagctgcagctgggctgtgtggttgacttatgggcccagccagccaggctcaggccatggctcccctttttcttcctcaccctgatttcttgcttattcactgaagttctcctgaagaggaactgggcctgttgccctttctgtaccatttatttgctcccaatgtttatgataataaaggcaccgctgatggggacctccactctgtctgtgtctgaggaggaactggcgcttttgcagaggcttactctgggctctgagcttgaaccagtgtggtctgtgctcagggccctgtgtttctgaaccgagggtgaggatgttgg

SEQ ID NO: 349 - DNA, 29 bp, Homo sapiens
attgagctaagaggagctgacaatatcag

SEQ ID NO: 350 - DNA, 94 bp, Homo sapiens
ctgtcctgggggaggtggacacctcgttggtgctgagctccatggaggaggccaagcagctggtggacaaggcctacaaggagcggcgggaaag

SEQ ID NO: 351 - DNA, 82 bp, Homo sapiens
gtggggcacgaggcactgcccagctctgggcaaggatgtcccaggcctttcagagaagcagcaggcagcagggagcttgaag

SEQ ID NO: 352 - DNA, 51 bp, Homo sapiens
gctcgcgcggtctccaacgagatcgtgcgcttccccactgatcagctgact

SEQ ID NO: 353 - DNA, 58 bp, Homo sapiens
gtcagctttggggtggggaccagaggtggcataggagatgttccctgttggagccaca

SEQ ID NO: 354 - DNA, 71 bp, Homo sapiens
gggacacccgttccagtgagatgcccgagctcacctccatgcacaccctcttacttcgggagcacaaccgg

SEQ ID NO: 355 - DNA, 90 bp, Homo sapiens
ctggccacagagctcaagagcctgaaccctaggtgggatggggagaggctctaccaggaagcccggaagatcgtgggggccatggtccag

SEQ ID NO: 356 - DNA, 30 bp, Homo sapiens
atcatcacttaccgggactacctgcccctg

SEQ ID NO: 357 - DNA, 104 bp, Homo sapiens
gtggcattgaccccatcctccggggcctcatggccacccctgccaagctgaatcgtcagaaccaaattgcagtggatgagatccgggagcgattgtttgagcag

SEQ ID NO: 358 - DNA, 67 bp, Homo sapiens
gtcatgaggattgggctggacctgcctgctctgaacatgcagcgcagcagggaccacggcctcccag

SEQ ID NO: 359 - DNA, 34 bp, Homo sapiens
gtacccagttcaggaagctccgggatggtgatcg

SEQ ID NO: 360 - DNA, 58 bp, Homo sapiens
gttttggtgggagaacgagggtgtgttcagcatgcagcagcgacaggccctggcccag

SEQ ID NO: 361 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 362 - protein, 650 aa, Homo sapiens
MELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 363 - protein, 58 aa, Homo sapiens [sequence omitted]

SEQ ID NO: 364 - protein, 777 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRCGWLGVAAGTGLREASRTPQASRCQRPVLPCRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 365 - protein, 724 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGFGGRTRVCSACSSDRPWPRSHCPGSSATTQASPPCLRTTSSCPTHIPGTLSTAVHFLH

SEQ ID NO: 366 - protein, 551 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGKQDLGQERVG

SEQ ID NO: 367 - protein, 429 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLADHHLPGLPAPGAGANGHEEVPAHVPFLQ

SEQ ID NO: 368 - protein, 426 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGQLWGGDQRWHRRCSLLEPQGHPFQ

SEQ ID NO: 369 - protein, 328 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKVALLPAHCLGWERGDCFWKGPSPFCAQVSFPRR

SEQ ID NO: 370 - protein, 143 aa, Homo sapiens
MGVPFFSSLRCMVDLGPCWAGGLTAEMKLLLALAGLLAILATPQPSEGAAPAVLGEVDTSLVLSSMEEAKQLVDKAYKERRERWGTRHCPALGKDVPGLSEKQQAAGSLKHQAAASQRLSQPHGTPILLQAAGGSHQDGGEGR

SEQ ID NO: 371 - protein, 810 aa, Homo sapiens
  misc_feature (5)..(5): Xaa can be any naturally occurring amino acid
  misc_feature (9)..(10): Xaa can be any naturally occurring amino acid
  misc_feature (30)..(30): Xaa can be any naturally occurring amino acid
  misc_feature (34)..(35): Xaa can be any naturally occurring amino acid
  misc_feature (52)..(52): Xaa can be any naturally occurring amino acid
  misc_feature (54)..(54): Xaa can be any naturally occurring amino acid
MYELXPERXXIDASELIGHTCHAINMYELXPERXXIDASEHEAVYCHAINHXMXSAPIENSHMANMGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 372 - protein, 5 aa, Artificial Sequence
  Synthetic peptide
GMARQ

SEQ ID NO: 373 - protein, 36 aa, Artificial Sequence
  Synthetic peptide
ALTTPWRGPTSCWYRSRTWVTRPQATRPLPPWKSPS

SEQ ID NO: 374 - protein, 4 aa, Artificial Sequence
  Synthetic peptide
FRPG

SEQ ID NO: 375 - protein, 42 aa, Artificial Sequence
  Synthetic peptide
AHCCDEPFSWRSMELGSNLLVPRCPQEKGTLEAEYTGSASEG

SEQ ID NO: 376 - protein, 22 aa, Artificial Sequence
  Synthetic peptide
DEPRLSASAPLVIHFLKAPPAP

SEQ ID NO: 377 - protein, 98 aa, Artificial Sequence
  Synthetic peptide
VPNLPCPSPSQTGSTSSGSCTTWGRSWPCSGSPSGLSSATPSPSDAVRRPPWFSATPPSRTTLTSRILDTAQSSGCISPTVGSTGSSCPGAMMSTCTR

SEQ ID NO: 378 - protein, 39 aa, Artificial Sequence
  Synthetic peptide
GEARGGQWGGGGRWGTVGGGGAEAVPAGDTLSPQSTCTR

SEQ ID NO: 379 - protein, 91 aa, Artificial Sequence
  Synthetic peptide
VGGGRGNAARPPGPVLQPQVSRWVALPAGFLHDPVPLLLPLAHGPRLPAAVQPAGPGHAALGAGVQQVRPLHQVPGPAGRGQAAALLPGAH

SEQ ID NO: 380 - protein, 16 aa, Artificial Sequence
  Synthetic peptide
RCSLLLRAGPPSLGGC

SEQ ID NO: 381 - protein, 60 aa, Artificial Sequence
  Synthetic peptide
KYAPLPAEGCCGSERGGWVGGLGVLHGAHRPSLLQQVRPLHQVPGPAGRGQAAALLPGAH

SEQ ID NO: 382 - protein, 25 aa, Artificial Sequence
  Synthetic peptide
QVRPLHQVPGPAGRGQAAALLPGAH

SEQ ID NO: 383 - protein, 118 aa, Artificial Sequence
  Synthetic peptide
VPNLPCPSPSQTGSTSSGSCTTWGRSWPCSGSPSGLSSATPSPSDAVRRPPWFSATPPSRTTLTSRILDTAQSSGCISPTVGSTGSSCPGAMMQVRPLHQVPGPAGRGQAAALLPGAH

SEQ ID NO: 384 - protein, 35 aa, Artificial Sequence
  Synthetic peptide
IGNIFPHSPHGRPSLLSLCSGQSSVTPSPTPPMAA

SEQ ID NO: 385 - protein, 37 aa, Artificial Sequence
  Synthetic peptide
VPSWTVSSPPTSSCTRTRQWTSSGASMPSLGASPTRK

SEQ ID NO: 386 - protein, 37 aa, Artificial Sequence; identical to SEQ ID NO: 385
  Synthetic peptide

SEQ ID NO: 387 - protein, 68 aa, Artificial Sequence
  Synthetic peptide
IVTKDMFIMEIDAICYYRMENASLLLSSLAHVSKAVQFLVQTTMKRLLAHRSLTEILLERKSIAQDAK

SEQ ID NO: 388 - protein, 8 aa, Artificial Sequence
  Synthetic peptide
LKTLQKEE

SEQ ID NO: 389 - protein, 68 aa, Artificial Sequence; identical to SEQ ID NO: 387
  Synthetic peptide

SEQ ID NO: 390 - protein, 29 aa, Artificial Sequence
  Synthetic peptide
ADPQRLHRDCAPLPLALPRCCSSRPAALR

SEQ ID NO: 391 - protein, 8 aa, Artificial Sequence
  Synthetic peptide
LKTLQKEE

SEQ ID NO: 392 - protein, 68 aa, Artificial Sequence; identical to SEQ ID NO: 387
  Synthetic peptide

SEQ ID NO: 393 - protein, 4 aa, Artificial Sequence
  Synthetic peptide
CTRV

SEQ ID NO: 394 - protein, 52 aa, Artificial Sequence
  Synthetic peptide
APEHKPGPVSSPPISSGLMTSPPISSVLRARHKPQISHRYLPPGAQAGMWGQ

SEQ ID NO: 395 - protein, 30 aa, Artificial Sequence
  Synthetic peptide
FVGQGGARMAETCVPVLRCNTAAPMWLNGT

SEQ ID NO: 396 - protein, 133 aa, Artificial Sequence
  Synthetic peptide
LDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTD

SEQ ID NO: 397 - protein, 47 aa, Artificial Sequence
  Synthetic peptide
QTQERMLGTDEGAQLTWRKKSAHCKLLAGTSPGLAVSWSNVAPSSAC

SEQ ID NO: 398 - protein, 30 aa, Artificial Sequence; identical to SEQ ID NO: 395
  Synthetic peptide

SEQ ID NO: 399 - protein, 133 aa, Artificial Sequence; identical to SEQ ID NO: 396
  Synthetic peptide

SEQ ID NO: 400 - protein, 30 aa, Artificial Sequence; identical to SEQ ID NO: 395
  Synthetic peptide

SEQ ID NO: 401 - protein, 133 aa, Artificial Sequence; identical to SEQ ID NO: 396
  Synthetic peptide

SEQ ID NO: 402 - protein, 34 aa, Artificial Sequence
  Synthetic peptide
RCGQRGSLGPLDGSNPKPLNHELPCQLPPTGSWE

SEQ ID NO: 403 - protein, 30 aa, Artificial Sequence; identical to SEQ ID NO: 395
  Synthetic peptide

SEQ ID NO: 404 - protein, 133 aa, Artificial Sequence; identical to SEQ ID NO: 396
  Synthetic peptide

SEQ ID NO: 405 - protein, 5 aa, Artificial Sequence
  Synthetic peptide
GGTSM

SEQ ID NO: 406 - protein, 858 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEGGTSM

SEQ ID NO: 407 - protein, 555 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQK

SEQ ID NO: 408 - protein, 4 aa, Artificial Sequence
  Synthetic peptide
GEYP

SEQ ID NO: 409 - protein, 862 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEGEYPDTKSG

SEQ ID NO: 410 - protein, 802 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKGSL

SEQ ID NO: 411 - protein, 3 aa, Artificial Sequence
  Synthetic peptide
GSL

SEQ ID NO: 412 - protein, 15 aa, Artificial Sequence
  Synthetic peptide
KCFQEHLEFGYSTAM

SEQ ID NO: 413 - protein, 814 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKKCFQEHLEFGYSTAM

SEQ ID NO: 414 - protein, 12 aa, Artificial Sequence
  Synthetic peptide
VRAIHGNYSCSV

SEQ ID NO: 415 - protein, 811 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKVRAIHGNYSCSV

SEQ ID NO: 416 - protein, 787 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHWTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 417 - protein, 805 aa, Artificial Sequence
  Synthetic peptide
AVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 418 - protein, 11 aa, Artificial Sequence
  Synthetic peptide
THGGGGGGGAF

SEQ ID NO: 419 - protein, 656 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGTHGGGGGGGAF

SEQ ID NO: 420 - protein, 13 aa, Artificial Sequence
  Synthetic peptide
CKYCIIHQSTKLF

SEQ ID NO: 421 - protein, 427 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHCKYCIIHQSTKLF

SEQ ID NO: 422 - protein, 8 aa, Artificial Sequence
  Synthetic peptide
EYTALCNK

SEQ ID NO: 423 - protein, 302 aa, Artificial Sequence
  Synthetic peptide
EKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSEYTALCNK

SEQ ID NO: 424 - protein, 4 aa, Artificial Sequence
  Synthetic peptide
GHLP

SEQ ID NO: 425 - protein, 16 aa, Artificial Sequence
  Synthetic peptide
CEWIHFWKSPRTLHVC

SEQ ID NO: 426 - protein, 58 aa, Artificial Sequence
  Synthetic peptide
FGGRTRVCSACSSDRPWPRSHCPGSSATTQASPPCLRTTSSCPTHIPGTLSTAVHFLH

SEQ ID NO: 427 - protein, 95 aa, Artificial Sequence
  Synthetic peptide
MGVPFFSSLRCMVDLGPCWAGGLTAEMKLLLALAGLLAILATPQPSEGAAPAVLGEVDTSLVLSSMEEAKQLVDKAYKERRESIKQRLRSGSASP

SEQ ID NO: 428 - protein, 10 aa, Artificial Sequence
  Synthetic peptide
KQDLGQERVG

SEQ ID NO: 429 - protein, 95 aa, Artificial Sequence; identical to SEQ ID NO: 427
  Synthetic peptide

SEQ ID NO: 430 - protein, 28 aa, Artificial Sequence
  Synthetic peptide
DHHLPGLPAPGAGANGHEEVPAHVPFLQ

SEQ ID NO: 431 - protein, 95 aa, Artificial Sequence; identical to SEQ ID NO: 427
  Synthetic peptide

SEQ ID NO: 432 - protein, 24 aa, Artificial Sequence
  Synthetic peptide
QLWGGDQRWHRRCSLLEPQGHPFQ

SEQ ID NO: 433 - protein, 95 aa, Artificial Sequence; identical to SEQ ID NO: 427
  Synthetic peptide

SEQ ID NO: 434 - protein, 33 aa, Artificial Sequence
  Synthetic peptide
VALLPAHCLGWERGDCFWKGPSPFCAQVSFPRR

SEQ ID NO: 435 - protein, 95 aa, Artificial Sequence; identical to SEQ ID NO: 427
  Synthetic peptide

SEQ ID NO: 436 - protein, 34 aa, Artificial Sequence
  Synthetic peptide
VCPPTVNQASPSSSLRLQTGMSQAQPTRIFDSTS

SEQ ID NO: 437 - protein, 61 aa, Artificial Sequence
  Synthetic peptide
RWGTRHCPALGKDVPGLSEKQQAAGSLKHQAAASQRLSQPHGTPILLQAAGGSHQDGGEGR

(19) United States
(12) Patent Application Publication
(10) Pub. No.: US 2009/0317808 A1
(43) Pub. Date: Dec. 24, 2009

(54) NOVEL NUCLEOTIDE AND AMINO ACID SEQUENCES AND METHODS OF USE THEREOF FOR DIAGNOSIS

(76) Inventors: Shirley Sameah-Greenwald, Kfar Saba (IL); Dan Sztybel, Tel-Aviv (IL); Osnat Sella-Tavor, Kfar Kish (IL); Sarah Pollock, Tel-Aviv (IL); Elena Tsypkin, Tel-Aviv (IL)

Correspondence Address: FENNEMORE CRAIG, 3003 NORTH CENTRAL AVENUE, SUITE 2600, PHOENIX, AZ, 85012, US

(21) Appl. No.: 12/293,068

(22) PCT Filed: Mar. 21, 2007
(86) PCT No.: PCT/IL2007/000370

§ 371 Date: Mar. 16, 2009

(30) Foreign Application Priority Data
Mar. 21, 2006 (IL) 174465

(51) Int. Cl. C12Q 1/68; C07H 21/00; C07K 14/00; C07K 16/00; C12N 15/74; C12N 1/20; C12P 21/00; G01N 33/53

(52) U.S. Cl. 435/6; 536/23.1; 530/324; 530/350; 530/387.9; 435/320.1; 435/252.33; 435/69.1; 435/7.92; 435/7.1

(57) ABSTRACT

Novel splice variants, amino acid sequences and nucleotide sequences thereof, and methods of using same.

### FIELD OF THE INVENTION

The present invention is related to novel nucleotide and protein sequences, and assays and methods of use thereof.

### BACKGROUND OF THE INVENTION

Diagnostic markers are important for early diagnosis of many diseases, as well as for disease predisposition, predicting a response to treatment, monitoring treatment progress and determining prognosis of the disease.

Serum markers are examples of diagnostic markers, and are used for diagnosis of many different diseases. Typically, serum markers encompass secreted proteins and/or peptides; however, some serum markers may be released to the blood upon tissue lysis, for example from myocardial infarction (Troponin-I being a specific example). Serum markers can also be used as indicative risk factors of a disease (for example base-line levels of CRP, as a predictor of cardiovascular disease); to monitor disease activity and progression (for example, determination of CRP levels to monitor acute phase inflammatory response); and to predict and monitor drug response (for example, as shedded fragments of the protein Erb-B2).

Immunohistochemistry (IHC) is the study of the distribution of an antigen of choice in a sample based on specific antibody-antigen binding, typically performed on tissue slices. The antibody features a label which can be detected, for example as a stain which is detectable under a microscope. Preparation of the tissue slices for the assay involves fixation; IHC is therefore particularly suitable for antibody-antigen reactions that are not disturbed or destroyed by the process of fixing the tissue slices.

IHC permits determining the localization of the bound antibody-antigen, and hence mapping the presence of the antigen within the tissue and even within different compartments in the cell. Such mapping can provide useful diagnostic information, including:

1) The histological type of the tissue sample
2) The presence of specific cell types within the sample
3) Information regarding the physiological and/or pathological state of cells (e.g. which phase of the cell-cycle they are in)
4) The presence of disease related changes within the sample
5) Differentiation between specific disease subtypes where it is already known that the tissue is diseased (for example, the differentiation between different tumor types when it is already known the sample was taken from cancerous tissue).

IHC information is valuable for more than diagnosis. It can also be used to determine prognosis and progression of a therapy treatment (for example, as in the case of HER-2 in breast cancer) as well as to monitor the disease state.

IHC protein markers could be from any cellular location. Most often these markers are membrane proteins but secreted proteins or intracellular proteins (including intranuclear) can also be used as an IHC marker.

Although widely used as diagnostic tool, the IHC technique has at least two major disadvantages. It is performed on tissue samples and therefore a tissue sample has to be collected from the patient, which most often requires invasive procedures like biopsy associated with pain, discomfort, hospitalization and risk of infection. In addition, the interpretation of the result is observer dependent and therefore subjective. There is no measured value but rather only estimation (on a scale of 1-4) of how prevalent the antigen is on the target.

Thus, there is a recognized need for, and it would be highly advantageous to have, an alternative diagnostic tool for diagnosing and monitoring diseases.

### SUMMARY OF THE INVENTION

The present invention provides novel nucleic acid and amino acid sequences, which can be used as diagnostic markers.

According to one aspect, the present invention provides a number of novel variants of known proteins which are found in serum and can be used as diagnostic markers. The present invention overcomes the many deficiencies of the background art with regard to the need to obtain tissue samples and subjective interpretations of results. In certain embodiments of the present invention, tissue specific markers are identifiable in serum or plasma. Thus, according to the teachings of the present invention, a simple blood test can provide qualitative and/or quantitative indication of various diseases and/or pathological conditions, according to the expression of certain marker(s).

According to another aspect, the present invention discloses the novel use of known proteins as diagnostic markers. In some embodiments, the markers disclosed can also be used for in-vivo imaging applications.

It is disclosed in the present invention for the first time that the protein variants of the invention are useful as diagnostic markers for various diseases and/or pathological conditions as described in greater detail below. The variants themselves are described by “cluster” or by gene, as these variants are splice variants of known proteins. Therefore, as used in the present invention, the term “marker-detectable disease” refers to a disease that may be detected by a particular marker, with regard to the description of the disease provided herein below. The markers of the present invention, alone or in combination, show a high degree of differential diagnosis between disease and non-disease states.

The present invention further relates to diagnostic assays for detecting a disease, particularly in a sample taken from a subject (patient), preferably a blood sample or a body secretion sample. According to certain embodiments, the diagnostic assays disclosed in the present invention are NAT (nucleic acid amplification technology)-based assays, including, for example, PCR or variations thereof, e.g. real-time PCR. According to other embodiments, the assays encompass nucleic acid hybridization assays. The diagnostic assays can be qualitative or quantitative.

According to certain embodiments, the present invention provides a diagnostic marker comprising a novel splice variant of a known protein or a polynucleotide encoding same, wherein the protein is selected from the group consisting of Cadherin-16 precursor (SwissProt accession identifier CADG_HUMAN (SEQ ID NO:89); known also according to the synonyms Kidney-specific cadherin; Ksp-cadherin); Inositol oxygenase (SwissProt accession identifier MIOX_HUMAN (SEQ ID NO:143); known also according to the synonyms EC 1.13.99.1; Myo-inositol oxygenase; Aldehyde reductase-like 6; Renal-specific oxidoreductase; Kidney-specific protein 32); Podocin (SwissProt accession identifier PODO_HUMAN (SEQ ID NO:178)); Uromodulin precursor (SwissProt accession identifier UROM_HUMAN (SEQ ID NO:242); known also according to the synonyms Tamm-Horsfall urinary glycoprotein; THP); Ceruloplasmin precursor (SwissProt accession identifier CERU_HUMAN (SEQ ID NO:308); known also according to the synonyms EC 1.16.3.1; Ferroxidase); and Myeloperoxidase precursor (SwissProt accession identifier PERM_HUMAN (SEQ ID NO:371); known also according to the synonyms EC 1.11.1.7; MPO). According to certain embodiments, the diagnostic marker is found in a body fluid or secretion.

According to one embodiment, the novel splice variant is an isolated polynucleotide comprising a nucleic acid having a nucleic acid sequence as set forth in any one of SEQ. ID NOs: 37-42, 105-119, 165-167, 190-193, 259-271, 329-334, or a sequence homologous thereto. According to one embodiment, the isolated polynucleotide is at least 85% homologous to any one of SEQ. ID NOs: 37-42, 105-119, 165-167, 190-193, 259-271, 329-334.

According to another embodiment, the novel splice variant is an isolated polynucleotide comprising a nucleic acid having a nucleic acid sequence as set forth in any one of SEQ. ID NOs: 43-88, 120-142, 168-177, 194-241, 272-307, 335-360, or a sequence homologous thereto. According to one embodiment, the isolated polynucleotide is at least 85% homologous to any one of SEQ. ID NOs: 43-88, 120-142, 168-177, 194-241, 272-307, 335-360.

According to certain embodiments, the present invention also encompasses isolated polynucleotides having a sequence complementary to any one of the nucleic acid sequences listed herein. According to other embodiments, this invention provides an oligonucleotide of at least about 12 nucleotides, specifically hybridizable with the polynucleotides of this invention. The present invention further provides vectors, cells, liposomes and compositions comprising the isolated polynucleotides of this invention.

According to yet another embodiment, the novel splice variant is an isolated protein or polypeptide having an amino acid sequence as set forth in any one of SEQ. ID NOs: 91-96, 145-149, 153-155, 180-183, 246, 247, 315, 318, 320-322, 365, 366, 368, 370, or a sequence homologous thereto. According to one embodiment, the isolated protein or polypeptide is at least 85% homologous to any one of SEQ. ID NOs: 91-96, 145-149, 153-155, 180-183, 246, 247, 315, 318, 320-322, 365, 366, 368, 370.

According to some embodiments, the sample taken from a subject (patient) to perform the diagnostic assay according to the present invention is selected from the group consisting of a body fluid or secretion including but not limited to blood, serum, urine, plasma, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, secretions of the breast ductal system (and/or lavage thereof), broncho alveolar lavage, lavage of the reproductive system and lavage of any other part of the body or system in the body; samples of any organ including isolated cell(s) or tissue(s), wherein the cell or tissue can be obtained from an organ selected from, but not limited to lung, colon, ovarian and/or breast tissue; stool or a tissue sample, or any combination thereof. In some embodiments, the term encompasses samples of in vivo cell culture constituents. Prior to be subjected to the diagnostic assay, the sample can optionally be diluted with a suitable eluant.

The term “homology”, as used herein, refers to a degree of sequence similarity in terms of shared amino acid or nucleotide sequences. There may be partial homology or complete homology (i.e., identity). For amino acid sequence homology amino acid similarity matrices may be used as are known in different bioinformatics programs (e.g. BLAST, Smith Waterman). Different results may be obtained when performing a particular search with a different matrix. Homologous peptide or polypeptides are characterized by one or more amino acid substitutions, insertions or deletions, such as, but not limited to, conservative substitutions, provided that these changes do not affect the biological activity of the peptide or polypeptide as described herein.

Degrees of homology for nucleotide sequences are based upon identity matches with penalties made for gaps or insertions required to optimize the alignment, as is well known in the art (e.g. Altschul S. F. et al., 1990, J Mol Biol 215(3):403-10; Altschul S. F. et al., 1997, Nucleic Acids Res. 25:3389-3402). The degree of sequence homology is presented in terms of percentage, e.g. “70% homology”. As used herein, the term “at least” with regard to a certain degree of homology encompasses any degree of homology from the specified percentage up to 100%.

The terms “correspond” or “corresponding to” or “correspondence with” are used herein to indicate identity between two corresponding amino acid or nucleic acid sequences.

In some embodiments, the proteins or polypeptides of this invention comprise chimeric protein or polypeptides.

As used herein, the terms “chimeric protein or polypeptide”, or “chimeric polynucleotide” or “chimera” refers to an assembly or a string of amino acids in a particular sequence, or nucleotides encoding the same, respectively, which does not correspond in their entirety to the sequence of the known (wild type) polypeptide or protein, or the nucleic acid encoding same.

In some embodiments, the variants of this invention are derived from two exons, or an exon and an intron of a known protein, or fragments thereof, or segments having sequences with the indicated homology.

According to certain embodiments, the present invention now discloses a novel cluster designated herein AA340453, comprising novel amino acid and nucleic acid sequences that are variants of the known protein Cadherin-16 precursor (SwissProt accession identifier CADG_HUMAN (SEQ ID NO:89); known also according to the synonyms Kidney-specific cadherin; Ksp-cadherin). The novel variant polynucleotides and polypeptides described by the present invention are useful as diagnostic markers, preferably as serum markers.

The present invention now discloses that AA340453 variants are specifically expressed in kidney tissues, and thus can be indicative of the presence, onset, severity or prognosis and/or staging and or progression of renal disease and/or condition, as described in a greater detail below.

According to one embodiment, the present invention provides an isolated polypeptide comprising an edge portion of AA340453_P27 (SEQ ID NO:91), comprising an amino acid sequence being at least about 70%, optionally at least about 80%, preferably at least about 85%, more preferably at least about 90% and most preferably at least about 95% homologous to the sequence GMARQ (SEQ ID NO:372) of AA340453_P27 (SEQ ID NO:91).

According to one embodiment, the isolated polypeptide is a chimeric polypeptide comprising a first amino acid sequence being at least about 90% homologous to amino acids 1-641 of CADG_HUMAN (SEQ ID NO:89), which also corresponds to amino acids 1-641 of AA340453_P27 (SEQ ID NO:91), and a second amino acid sequence being at least about 70%, optionally at least about 80%, preferably at least about 85%, more preferably at least about 90% and most preferably at least about 95% homologous to a polypeptide having the sequence GMARQ (SEQ ID NO:372) corresponding to amino acids 642-646 of AA340453_P27 (SEQ ID NO:91), wherein said first amino acid sequence and second amino acid sequence are contiguous and in a sequential order.

According to another embodiment, the isolated polypeptide comprises an amino acid sequence as set forth in SEQ. ID NO: 91 (AA340453_P27).

According to one embodiment, the isolated polypeptide is a chimeric polypeptide comprising a first amino acid sequence being at least about 90% homologous to amino acids 1-301 of CADG_HUMAN (SEQ ID NO:89), which also corresponds to amino acids 1-301 of AA340453_P30 (SEQ ID NO:93), and a second amino acid sequence being having the sequence VI corresponding to amino acids 302-303 of AA340453_P30 (SEQ ID NO:93), wherein said first amino acid sequence and second amino acid sequence are contiguous and in a sequential order.

According to another embodiment, the isolated polypeptide comprises an amino acid sequence as set forth in SEQ. ID NO: 93 (AA340453_P30).

According to one embodiment, the present invention provides an isolated polypeptide comprising an edge portion of AA340453_P31 (SEQ ID NO:94), comprising an amino acid sequence being at least about 70%, optionally at least about 80%, preferably at least about 85%, more preferably at least about 90% and most preferably at least about 95% homologous to the sequence ALTTPWRGPTSCWYRSRTWVTRPQATRPLPPWKSPS (SEQ ID NO:373).

According to one embodiment, the isolated polypeptide is a chimeric polypeptide comprising a first amino acid sequence being at least about 90% homologous to amino acids 1-194 of CADG_HUMAN (SEQ ID NO:89), which also corresponds to amino acids 1-194 of AA340453_P31 (SEQ ID NO:94), and a second amino acid sequence being at least about 70%, optionally at least about 80%, preferably at least about 85%, more preferably at least about 90% and most preferably at least about 95% homologous to a polypeptide having the sequence ALTTPWRGPTSCWYRSRTWVTRPQATRPLPPWKSPS (SEQ ID NO:373) corresponding to amino acids 195-230 of AA340453_P31 (SEQ ID NO:94), wherein said first amino acid sequence and second amino acid sequence are contiguous and in a sequential order.

According to another embodiment, the isolated polypeptide comprises an amino acid sequence as set forth in SEQ. ID NO: 94 (AA340453_P31).

According to one embodiment, the present invention provides an isolated polypeptide comprising an edge portion of AA340453_P32 (SEQ ID NO:95), comprising an amino acid sequence being at least about 70%, optionally at least about 80%, preferably at least about 85%, more preferably at least about 90% and most preferably at least about 95% homologous to the sequence FRPG (SEQ ID NO:374) of AA340453_P32 (SEQ ID NO:95).

According to one embodiment, the isolated polypeptide is a chimeric polypeptide comprising a first amino acid sequence being at least about 90% homologous to corresponding to amino acids 1-142 of CADG_HUMAN (SEQ ID NO:89), which also corresponds to amino acids 1-142 of AA340453_P32 (SEQ ID NO:95), and a second amino acid sequence being at least about 70%, optionally at least about 80%, preferably at least about 85%, more preferably at least about 90% and most preferably at least about 95% homologous to a polypeptide having the sequence FRPG (SEQ ID NO:374) corresponding to amino acids 143-146 of AA340453_P32 (SEQ ID NO:95), wherein said first amino acid sequence and second amino acid sequence are contiguous and in a sequential order.

According to another embodiment, the isolated polypeptide comprises an amino acid sequence as set forth in SEQ. ID NO: 95 (AA340453_P32).

According to one embodiment, the present invention provides an isolated polypeptide comprising an edge portion of AA340453_P34 (SEQ ID NO:96), comprising an amino acid sequence being at least about 70%, optionally at least about 80%, preferably at least about 85%, more preferably at least about 90% and most preferably at least about 95% homologous to the sequence AHCCDEPFSWRSMELGSNLLVPRCPQEKGTLEAEYTGSASEG (SEQ ID NO:375) of AA340453_P34 (SEQ ID NO:96).

According to one embodiment, the isolated polypeptide is a chimeric polypeptide comprising a first amino acid sequence being at least about 90% homologous to corresponding to amino acids 1-797 of CADG_HUMAN (SEQ ID NO:89), which also corresponds to amino acids 1-797 of AA340453_P34 (SEQ ID NO:96), and a second amino acid sequence being at least about 70%, optionally at least about 80%, preferably at least about 85%, more preferably at least about 90% and most preferably at least about 95% homologous to a polypeptide having the sequence AHCCDEPFSWRSMELGSNLLVPRCPQEKGTLEAEYTGSASEG (SEQ ID NO:375) corresponding to amino acids 798-839 of AA340453_P34 (SEQ ID NO:96), wherein said first amino acid sequence and second amino acid sequence are contiguous and in a sequential order.

According to one embodiment, the present invention provides an isolated polypeptide comprising an edge portion of AA340453_P34 (SEQ ID NO:96), comprising an amino acid sequence being at least about 70%, optionally at least about 80%, preferably at least about 85%, more preferably at least about 90% and most preferably at least about 95% homologous to the sequence DEPRLSASAPLVIHFLKAPPAP (SEQ ID NO:376) of AA340453_P34 (SEQ ID NO:96).

According to one embodiment, the present invention provides an isolated polypeptide comprising an edge portion of AA340453_P34 (SEQ ID NO:96), comprising an amino acid sequence being at least about 70%, optionally at least about 80%, preferably at least about 85%, more preferably at least about 90% and most preferably at least about 95% homologous to the sequence AHCCDEPFSWRSMELGSNLLVPRCPQEKGTLEAEYTGSASEG (SEQ ID NO:375) of AA340453_P34 (SEQ ID NO:96).

According to one embodiment, the isolated polypeptide is a chimeric polypeptide comprising a first amino acid sequence being at least about 90% homologous to amino acids 1-641 of Q6UW93_HUMAN (SEQ ID NO:90), which also corresponds to amino acids 1-641 of AA340453_P34 (SEQ ID NO:96), a second amino acid sequence being at least about 70%, optionally at least about 80%, preferably at least about 85%, more preferably at least about 90% and most preferably at least about 95% homologous to a polypeptide having the sequence DEPRLSASAPLVIHFLKAPPAP (SEQ ID NO:376) corresponding to amino acids 642-663 of AA340453_P34 (SEQ ID NO:96), a third amino acid sequence being at least about 90% homologous to amino acids 642-775 of Q6UW93_HUMAN (SEQ ID NO:90), which also corresponds to amino acids 664-797 of AA340453_P34 (SEQ ID NO:96), and a fourth amino acid sequence being at least about 70%, optionally at least about 80%, preferably at least about 85%, more preferably at least about 90% and most preferably at least about 95% homologous to a polypeptide having the sequence AHCCDEPFSWRSMELGSNLLVPRCPQEKGTLEAEYTGSASEG (SEQ ID NO:375) corresponding to amino acids 798-839 of AA340453_P34 (SEQ ID NO:96), wherein said first amino acid sequence, second amino acid sequence, third amino acid sequence and fourth amino acid sequence are contiguous and in a sequential order.

According to another embodiment, the isolated polypeptide comprises an amino acid sequence as set forth in SEQ. ID NO: 96 (AA340453_P34).

According to certain embodiments, the present invention now discloses a novel cluster designated herein AA703666, comprising novel amino acid and nucleic acid sequences that are variants of the known protein Inositol oxygenase (SwissProt accession identifier MIOX_HUMAN (SEQ ID NO:143); known also according to the synonyms EC 1.13.99.1; Myo-inositol oxygenase; Aldehyde reductase-like 6; Renal-specific oxidoreductase; Kidney-specific protein 32). The novel variant polynucleotides and polypeptides described by the present invention are useful as diagnostic markers, preferably as serum markers.

The present invention now discloses that AA703666 variants are specifically expressed in kidney tissues, and thus can be indicative of the presence, onset, severity or prognosis and/or staging and or progression of renal disease and/or condition, as described in a greater detail below.

According to one embodiment, the present invention provides an isolated polypeptide comprising an edge portion of AA703666_P1 (SEQ ID NO:144), comprising an amino acid sequence being at least about 70%, optionally at least about 80%, preferably at least about 85%, more preferably at least about 90% and most preferably at least about 95% homologous to the sequence VPNLPCPSPSQTGSTSSGSCTTWGRSWPCSGSPSGLSSATPSPSDAVRRPPWFSATPPSRTT LTSRILDTAQSSGCISPTVGSTGSSCPGAMMSTCTR (SEQ ID NO:377) of AA703666_P1 (SEQ ID NO:144).

According to one embodiment, the isolated polypeptide is a chimeric polypeptide comprising a first amino acid sequence being at least about 90% homologous to amino acids 1-113 of MIOX_HUMAN (SEQ ID NO:143), which also corresponds to amino acids 1-113 of AA703666_P1 (SEQ ID NO:144), and a second amino acid sequence being at least about 70%, optionally at least about 80%, preferably at least about 85%, more preferably at least about 90% and most preferably at least about 95% homologous to a polypeptide having the sequence VPNLPCPSPSQTGSTSSGSCTTWGRSWPCSGSPSGLSSATPSPSDAVRRPPWFSATPPSRTT LTSRILDTAQSSGCISPTVGSTGSSCPGAMMSTCTR (SEQ ID NO:377) corresponding to amino acids 114-211 of AA703666_P1 (SEQ ID NO:144), wherein said first amino acid sequence and second amino acid sequence are contiguous and in a sequential order.

According to one embodiment, the present invention provides an isolated polypeptide comprising an edge portion of AA703666_P3 (SEQ ID NO:145), comprising an amino acid sequence being at least about 70%, optionally at least about 80%, preferably at least about 85%, more preferably at least about 90% and most preferably at least about 95% homologous to the sequence GEARGGQWGGGGRWGTVGGGGAEAVPAGDTLSPQSTCTR (SEQ ID NO:378) of AA703666_P3 (SEQ ID NO:145).

According to one embodiment, the isolated polypeptide is a chimeric polypeptide comprising a first amino acid sequence being at least about 90% homologous to amino acids 1-195 of MIOX_HUMAN (SEQ ID NO:143), which also corresponds to amino acids 1-195 of AA703666_P3 (SEQ ID NO:145), and a second amino acid sequence being at least about 70%, optionally at least about 80%, preferably at least about 85%, more preferably at least about 90% and most preferably at least about 95% homologous to a polypeptide having the sequence GEARGGQWGGGGRWGTVGGGGAEAVPAGDTLSPQSTCTR (SEQ ID NO:378) corresponding to amino acids 196-234 of AA703666_P3 (SEQ ID NO:145), wherein said first amino acid sequence and second amino acid sequence are contiguous and in a sequential order.

According to another embodiment, the isolated polypeptide comprises an amino acid sequence as set forth in SEQ. ID NO: 145 (AA703666_P3).

According to one embodiment, the present invention provides an isolated polypeptide comprising an edge portion of AA703666_P4 (SEQ ID NO:146), comprising an amino acid sequence being at least about 70%, optionally at least about 80%, preferably at least about 85%, more preferably at least about 90% and most preferably at least about 95% homologous to the sequence VGGGRGNAARPPGPVLQPQVSRWVALPAGFLHDPVPLLLPLAHGPRLPAAVQPAGPGH AALGAGVQQVRPLHQVPGPAGRGQAAALLPGAH (SEQ ID NO:379) of AA703666_P4 (SEQ ID NO:146).

According to one embodiment, the isolated polypeptide is a chimeric polypeptide comprising a first amino acid sequence being at least about 90% homologous to amino acids 1-212 of MIOX_HUMAN (SEQ ID NO:143), which also corresponds to amino acids 1-212 of AA703666_P4 (SEQ ID NO:146), and a second amino acid sequence being at least about 70%, optionally at least about 80%, preferably at least about 85%, more preferably at least about 90% and most preferably at least about 95% homologous to a polypeptide having the sequence VGGGRGNAARPPGPVLQPQVSRWVALPAGFLHDPVPLLLPLAHGPRLPAAVQPAGPGH AALGAGVQQVRPLHQVPGPAGRGQAAALLPGAH (SEQ ID NO:379) corresponding to amino acids 213-303 of AA703666_P4 (SEQ ID NO:146), wherein said first amino acid sequence and second amino acid sequence are contiguous and in a sequential order.

According to another embodiment, the isolated polypeptide comprises an amino acid sequence as set forth in SEQ. ID NO: 146 (AA703666_P4).

According to one embodiment, the present invention provides an isolated polypeptide comprising an edge portion of AA703666_P5 (SEQ ID NO:147), comprising an amino acid sequence being at least about 70%, optionally at least about 80%, preferably at least about 85%, more preferably at least about 90% and most preferably at least about 95% homologous to the sequence RCSLLLRAGPPSLGGC (SEQ ID NO:380) of AA703666_P5 (SEQ ID NO:147).

According to one embodiment, the isolated polypeptide is a chimeric polypeptide comprising a first amino acid sequence being at least about 90% homologous to amino acids 1-172 of MIOX_HUMAN (SEQ ID NO:143), which also corresponds to amino acids 1-172 of AA703666_P5 (SEQ ID NO:147), and a second amino acid sequence being at least about 70%, optionally at least about 80%, preferably at least about 85%, more preferably at least about 90% and most preferably at least about 95% homologous to a polypeptide having the sequence RCSLLLRAGPPSLGGC (SEQ ID NO:380) corresponding to amino acids 173-188 of AA703666_P5 (SEQ ID NO:147), wherein said first amino acid sequence and second amino acid sequence are contiguous and in a sequential order.

According to another embodiment, the isolated polypeptide comprises an amino acid sequence as set forth in SEQ. ID NO: 147 (AA703666_P5).

According to one embodiment, the present invention provides an isolated polypeptide comprising an edge portion of AA703666_P6 (SEQ ID NO:148), comprising an amino acid sequence being at least about 70%, optionally at least about 80%, preferably at least about 85%, more preferably at least about 90% and most preferably at least about 95% homologous to the sequence KYAPLPAEGCCGSERGGWVGGLGVLHGAHRPSLLQQVRPLHQVPGPAGRGQAAALLPG AH (SEQ ID NO:381) of AA703666_P6 (SEQ ID NO:148).

According to one embodiment, the isolated polypeptide is a chimeric polypeptide comprising a first amino acid sequence being at least about 90% homologous to amino acids 1-249 of MIOX_HUMAN (SEQ ID NO:143), which also corresponds to amino acids 1-249 of AA703666_P6 (SEQ ID NO:148), and a second amino acid sequence being at least about 70%, optionally at least about 80%, preferably at least about 85%, more preferably at least about 90% and most preferably at least about 95% homologous to a polypeptide having the sequence KYAPLPAEGCCGSERGGWVGGLGVLHGAHRPSLLQQVRPLHQVPGPAGRGQAAALLPG AH (SEQ ID NO:381) corresponding to amino acids 250-309 of AA703666_P6 (SEQ ID NO:148), wherein said first amino acid sequence and second amino acid sequence are contiguous and in a sequential order.

According to another embodiment, the isolated polypeptide comprises an amino acid sequence as set forth in SEQ. ID NO: 148 AA703666_P6 (SEQ ID NO:148).

According to one embodiment, the present invention provides an isolated polypeptide comprising an edge portion of AA703666_P7 (SEQ ID NO:149), comprising an amino acid sequence being at least about 70%, optionally at least about 80%, preferably at least about 85%, more preferably at least about 90% and most preferably at least about 95% homologous to the sequence QVRPLHQVPGPAGRGQAAALLPGAH (SEQ ID NO:382) of AA703666_P7 (SEQ ID NO:149).

According to one embodiment, the isolated polypeptide is a chimeric polypeptide comprising a first amino acid sequence being at least about 90% homologous to amino acids 1-245 of MIOX_HUMAN (SEQ ID NO:143), which also corresponds to amino acids 1-245 of AA703666_P7 (SEQ ID NO:149), and a second amino acid sequence being at least about 70%, optionally at least about 80%, preferably at least about 85%, more preferably at least about 90% and most preferably at least about 95% homologous to a polypeptide having the sequence QVRPLHQVPGPAGRGQAAALLPGAH (SEQ ID NO:382) corresponding to amino acids 246-270 of AA703666_P7 (SEQ ID NO:149), wherein said first amino acid sequence and second amino acid sequence are contiguous and in a sequential order.

According to another embodiment, the isolated polypeptide comprises an amino acid sequence as set forth in SEQ. ID NO: 149 (AA703666_P7).

According to one embodiment, the isolated polypeptide is a chimeric polypeptide comprising a first amino acid sequence being at least about 90% homologous to amino acids 1-136 of MIOX_HUMAN (SEQ ID NO:143), which also corresponds to amino acids 1-136 of AA703666_P9 (SEQ ID NO:150), and a second amino acid sequence being at least about 90% homologous to amino acids 152-285 of MIOX_HUMAN (SEQ ID NO:143), which also corresponds to amino acids 137-270 of AA703666_P9 (SEQ ID NO:150), wherein said first amino acid sequence and second amino acid sequence are contiguous and in a sequential order.

According to one embodiment, the present invention provides an isolated polypeptide comprising an edge portion of AA703666_P9 (SEQ ID NO:150), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise QA, having a structure as follows: a sequence starting from any of amino acid numbers 136-x to 136; and ending at any of amino acid numbers 137+((n−2)−x), in which x varies from 0 to n−2.

According to one embodiment, the isolated polypeptide is a chimeric polypeptide comprising a first amino acid sequence being at least about 90% homologous to amino acids 1-32 of MIOX_HUMAN (SEQ ID NO:143), which also corresponds to amino acids 1-32 of AA703666_P10 (SEQ ID NO:151), and a second amino acid sequence being at least about 90% homologous to amino acids 60-285 of MIOX_HUMAN (SEQ ID NO:143), which also corresponds to amino acids 33-258 of AA703666_P10 (SEQ ID NO:151), wherein said first amino acid sequence and second amino acid sequence are contiguous and in a sequential order.

According to one embodiment, the present invention provides an isolated polypeptide comprising an edge portion of AA703666_P10 (SEQ ID NO:151), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise TH, having a structure as follows: a sequence starting from any of amino acid numbers 32-x to 32; and ending at any of amino acid numbers 33+((n−2)−x), in which x varies from 0 to n−2.

According to one embodiment, the present invention provides an isolated polypeptide comprising an edge portion of AA703666_P12 (SEQ ID NO:152), comprising an amino acid sequence being at least about 70%, optionally at least about 80%, preferably at least about 85%, more preferably at least about 90% and most preferably at least about 95% homologous to the sequence VPNLPCPSPSQTGSTSSGSCTTWGRSWPCSGSPSGLSSATPSPSDAVRRPPWFSATPPSRTT LTSRILDTAQSSGCISPTVGSTGSSCPGAMMQVRPLHQVPGPAGRGQAAALLPGAH (SEQ ID NO:383) of AA703666_P12 (SEQ ID NO:152).

According to one embodiment, the isolated polypeptide is a chimeric polypeptide comprising a first amino acid sequence being at least about 90% homologous to amino acids 1-113 of MIOX_HUMAN (SEQ ID NO:143), which also corresponds to amino acids 1-113 of AA703666_P12 (SEQ ID NO:152), and a second amino acid sequence being at least about 70%, optionally at least about 80%, preferably at least about 85%, more preferably at least about 90% and most preferably at least about 95% homologous to a polypeptide having the sequence VPNLPCPSPSQTGSTSSGSCTTWGRSWPCSGSPSGLSSATPSPSDAVRRPPWFSATPPSRTT LTSRILDTAQSSGCISPTVGSTGSSCPGAMMQVRPLHQVPGPAGRGQAAALLPGAH (SEQ ID NO:383) corresponding to amino acids 114-231 of AA703666_P12 (SEQ ID NO:152), wherein said first amino acid sequence and second amino acid sequence are contiguous and in a sequential order.

According to one embodiment, the present invention provides an isolated polypeptide comprising an edge portion of AA703666_P13 (SEQ ID NO:153), comprising an amino acid sequence being at least about 70%, optionally at least about 80%, preferably at least about 85%, more preferably at least about 90% and most preferably at least about 95% homologous to the sequence IGNIFPHSPHGRPSLLSLCSGQSSVTPSPTPPMAA (SEQ ID NO:384) of AA703666_P13 (SEQ ID NO:153).

According to one embodiment, the isolated polypeptide is a chimeric polypeptide comprising a first amino acid sequence being at least about 90% homologous to amino acids 1-139 of MIOX_HUMAN (SEQ ID NO:143), which also corresponds to amino acids 1-139 of AA703666_P13 (SEQ ID NO:153), and a second amino acid sequence being at least about 70%, optionally at least about 80%, preferably at least about 85%, more preferably at least about 90% and most preferably at least about 95% homologous to a polypeptide having the sequence IGNIFPHSPHGRPSLLSLCSGQSSVTPSPTPPMAA (SEQ ID NO:384) corresponding to amino acids 140-174 of AA703666_P13 (SEQ ID NO:153), wherein said first amino acid sequence and second amino acid sequence are contiguous and in a sequential order.

According to another embodiment, the isolated polypeptide comprises an amino acid sequence as set forth in SEQ. ID NO:153 (AA703666_P13).

According to one embodiment, the present invention provides an isolated polypeptide comprising an edge portion of AA703666P115 (SEQ ID NO:154), comprising an amino acid sequence being at least about 70%, optionally at least about 80%, preferably at least about 85%, more preferably at least about 90% and most preferably at least about 95% homologous to the sequence VPSWTVSSPPTSSCTRTRQWTSSGASMPSLGASPTRK (SEQ ID NO:386) of AA703666_P15 (SEQ ID NO:154).

According to one embodiment, the isolated polypeptide is a chimeric polypeptide comprising a first amino acid sequence being at least about 90% homologous to amino acids 1-32 of MIOX_HUMAN (SEQ ID NO:143), which also corresponds to amino acids 1-32 of AA703666_P15 (SEQ ID NO:154), and a second amino acid sequence being at least about 70%, optionally at least about 80%, preferably at least about 85%, more preferably at least about 90% and most preferably at least about 95% homologous to a polypeptide having the sequence VPSWTVSSPPTSSCTRTRQWTSSGASMPSLGASPTRK (SEQ ID NO:385) corresponding to amino acids 33-69 of AA703666_P15 (SEQ ID NO:154), wherein said first amino acid sequence and second amino acid sequence are contiguous and in a sequential order.

According to another embodiment, the isolated polypeptide comprises an amino acid sequence as set forth in SEQ. ID NO: 154 (AA703666_P15).

According to one embodiment, the present invention provides an isolated polypeptide comprising an edge portion of AA703666_P16 (SEQ ID NO:155), comprising an amino acid sequence being at least about 70%, optionally at least about 80%, preferably at least about 85%, more preferably at least about 90% and most preferably at least about 95% homologous to the sequence VPSWTVSSPPTSSCTRTRQWTSSGASMPSLGASPTRK (SEQ ID NO:385) of AA703666_P16 (SEQ ID NO:155).

According to one embodiment, the isolated polypeptide is a chimeric polypeptide comprising a first amino acid sequence of MKVT corresponding to amino acids 1-4 of MIOX_HUMAN (SEQ ID NO:143), which also corresponds to amino acids 1-4 of AA703666_P16 (SEQ ID NO:155), a second amino acid sequence being at least about 90% homologous to GPDPSLVYRPDVDPEVAKDKASFRNYT corresponding to amino acids 6-32 of MIOX_HUMAN (SEQ ID NO:143), which also corresponds to amino acids 5-31 of AA703666_P16 (SEQ ID NO:155), and a third amino acid sequence being at least about 70%, optionally at least about 80%, preferably at least about 85%, more preferably at least about 90% and most preferably at least about 95% homologous to a polypeptide having the sequence VPSWTVSSPPTSSCTRTRQWTSSGASMPSLGASPTRK (SEQ ID NO:385) corresponding to amino acids 32-68 of AA703666_P16 (SEQ ID NO:155), wherein said first amino acid sequence, second amino acid sequence and third amino acid sequence are contiguous and in a sequential order.

According to another embodiment, the isolated polypeptide comprises an amino acid sequence as set forth in SEQ. ID NO: 155 (AA703666_P16).

According to certain embodiments, the present invention now discloses a novel cluster designated herein AI590292, comprising novel amino acid and nucleic acid sequences that are variants of the known protein Podocin (SwissProt accession identifier PODO_HUMAN (SEQ ID NO:178). The novel variant polynucleotides and polypeptides described by the present invention are useful as diagnostic markers, preferably as serum markers.

The present invention now discloses that AI590292 variants are specifically expressed in kidney tissues, and thus can be indicative of the presence, onset, severity or prognosis and/or staging and or progression of renal disease and/or condition, as described in a greater detail below.

According to one embodiment, the isolated polypeptide is a chimeric polypeptide comprising a first amino acid sequence being at least about 90% homologous to amino acids 1-91 of PODO_HUMAN (SEQ ID NO:178), which also corresponds to amino acids 1-91 of AI590292_P5 (SEQ ID NO:180), and a second amino acid sequence being at least about 90% homologous to amino acids 151-383 of PODO_HUMAN (SEQ ID NO:178), which also corresponds to amino acids 92-324 of AI590292_P5 (SEQ ID NO:180), wherein said first amino acid sequence and second amino acid sequence are contiguous and in a sequential order.

According to one embodiment, the present invention provides an isolated polypeptide comprising an edge portion of AI590292_P5 (SEQ ID NO:180), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise EG, having a structure as follows: a sequence starting from any of amino acid numbers 91-x to 91; and ending at any of amino acid numbers 92+((n−2)−x), in which x varies from 0 to n−2.

AI590292_P5 (SEQ ID NO:180) amino acid sequence being at least about 90% homologous to amino acids 1-91 of Q9NP85-2 (SEQ ID NO:179), which also corresponds to amino acids 1-91 of AI590292_P5 (SEQ ID NO:180), a second amino acid sequence being at least about 90% homologous to amino acids 151-178 of Q9NP85-2 (SEQ ID NO:179), which also corresponds to amino acids 92-119 of AI590292_P5 (SEQ ID NO:180), a third amino acid sequence being at least about 70%, optionally at least about 80%, preferably at least about 85%, more preferably at least about 90% and most preferably at least about 95% homologous to a polypeptide having the sequence IVTKDMFIMEIDAICYYRMENASLLLSSLAHVSKAVQFLVQTTMKRLLAHRSLTEILLER KSIAQDAK (SEQ ID NO:387) corresponding to amino acids 120-187 of AI590292_P5 (SEQ ID NO:180), and a fourth amino acid sequence being at least about 90% homologous to amino acids 179-315 of Q9NP85-2 (SEQ ID NO:179), which also corresponds to amino acids 188-324 of AI590292_P5 (SEQ ID NO:180), wherein said first amino acid sequence, second amino acid sequence, third amino acid sequence and fourth amino acid sequence are contiguous and in a sequential order.

According to one embodiment, the present invention provides an isolated polypeptide comprising an edge portion of AI590292_P5 (SEQ ID NO:180), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise EG, having a structure as follows: a sequence starting from any of amino acid numbers 91-x to 91; and ending at any of amino acid numbers 92+((n−2)−x), in which x varies from 0 to n−2.

According to one embodiment, the present invention provides an isolated polypeptide comprising an edge portion of AI590292_P5 (SEQ ID NO:180), comprising an amino acid sequence being at least about 70%, optionally at least about 80%, preferably at least about 85%, more preferably at least about 90% and most preferably at least about 95% homologous to the sequence IVTKDMFIMEIDAICYYRMENASLLLSSLAHVSKAVQFLVQTTMKRLLAHRSLTEILLER KSIAQDAK (SEQ ID NO:387) of AI590292_P5 (SEQ ID NO:180).

According to another embodiment, the isolated polypeptide comprises an amino acid sequence as set forth in SEQ. ID NO: 180 (AI590292_P5).

According to one embodiment, the isolated polypeptide is a chimeric polypeptide comprising a first amino acid sequence being at least about 90% homologous to amino acids 1-91 of PODO_HUMAN (SEQ ID NO:178), which also corresponds to amino acids 1-91 of AI590292_P6 (SEQ ID NO:181), a second amino acid sequence being at least about 90% homologous to amino acids 151-291 of PODO_HUMAN (SEQ ID NO:178), which also corresponds to amino acids 92-232 of AI590292_P6 (SEQ ID NO:181), and a third amino acid sequence being at least about 70%, optionally at least about 80%, preferably at least about 85%, more preferably at least about 90% and most preferably at least about 95% homologous to a polypeptide having the sequence LKTLQKEE (SEQ ID NO:388) corresponding to amino acids 233-240 of AI590292_P6 (SEQ ID NO:181), wherein said first amino acid sequence, second amino acid sequence and third amino acid sequence are contiguous and in a sequential order.

According to one embodiment, the present invention provides an isolated polypeptide comprising an edge portion of AI590292_P6 (SEQ ID NO:181), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise EG, having a structure as follows: a sequence starting from any of amino acid numbers 91-x to 91; and ending at any of amino acid numbers 92+((n−2)−x), in which x varies from 0 to n−2.

According to one embodiment, the present invention provides an isolated polypeptide comprising an edge portion of AI590292_P6 (SEQ ID NO:181), comprising an amino acid sequence being at least about 70%, optionally at least about 80%, preferably at least about 85%, more preferably at least about 90% and most preferably at least about 95% homologous to the sequence LKTLQKEE (SEQ ID NO:388) of AI590292_P6 (SEQ ID NO:181).

According to one embodiment, the isolated polypeptide is a chimeric polypeptide comprising a first amino acid sequence being at least about 90% homologous to MERRARSSSRESRGRGGRTPHKENKRAKAERSGGGRGRQEAGPEPSGSGRAGTPGEPRA PAATVVDVDEVRGSGEEGTEVVALLESERPEE corresponding to amino acids 1-91 of Q9NP85-2 (SEQ ID NO:179), which also corresponds to amino acids 1-91 of AI590292_P6 (SEQ ID NO:181), a second amino acid sequence being at least about 90% homologous to GLFFFLPCLDTYHKVDLRLQTLEIPFHE corresponding to amino acids 151-178 of Q9NP85-2 (SEQ ID NO:179), which also corresponds to amino acids 92-119 of AI590292_P6 (SEQ ID NO:181), a third amino acid sequence being at least about 70%, optionally at least about 80%, preferably at least about 85%, more preferably at least about 90% and most preferably at least about 95% homologous to a polypeptide having the sequence IVTKDMFIMEIDAICYYRMENASLLLSSLAHVSKAVQFLVQTTMKRLLAHRSLTEILLER KSIAQDAK (SEQ ID NO:389) corresponding to amino acids 120-187 of AI590292_P6 (SEQ ID NO:181), a fourth amino acid sequence being at least about 90% homologous to VALDSVTCIWGIKVERIEIKDVRLPAGLQHSLAVEAEAQRQAKVR corresponding to amino acids 179-223 of Q9NP85-2 (SEQ ID NO:179), which also corresponds to amino acids 188-232 of AI590292_P6 (SEQ ID NO:181), and a fifth amino acid sequence being at least about 70%, optionally at least about 80%, preferably at least about 85%, more preferably at least about 90% and most preferably at least about 95% homologous to a polypeptide having the sequence LKTLQKEE (SEQ ID NO:391) corresponding to amino acids 233-240 of AI590292_P6 (SEQ ID NO:181), wherein said first amino acid sequence, second amino acid sequence, third amino acid sequence, fourth amino acid sequence and fifth amino acid sequence are contiguous and in a sequential order.

According to one embodiment, the present invention provides an isolated polypeptide comprising an edge portion of AI590292_P6 (SEQ ID NO:181), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise EG, having a structure as follows: a sequence starting from any of amino acid numbers 91-x to 91; and ending at any of amino acid numbers 92+((n−2)−x), in which x varies from 0 to n−2.

According to one embodiment, the present invention provides an isolated polypeptide comprising an edge portion of AI590292_P6 (SEQ ID NO:181), comprising an amino acid sequence being at least about 70%, optionally at least about 80%, preferably at least about 85%, more preferably at least about 90% and most preferably at least about 95% homologous to the sequence IVTKDMFIMEIDAICYYRMENASLLLSSLAHVSKAVQFLVQTTMKRLLAHRSLTEILLER KSIAQDAK (SEQ ID NO:392) of AI590292_P6 (SEQ ID NO:181).

According to one embodiment, the present invention provides an isolated polypeptide comprising an edge portion of AI590292_P6 (SEQ ID NO:181), comprising an amino acid sequence being at least about 70%, optionally at least about 80%, preferably at least about 85%, more preferably at least about 90% and most preferably at least about 95% homologous to the sequence LKTLQKEE (SEQ ID NO:388) of AI590292_P6 (SEQ ID NO:181).

According to another embodiment, the isolated polypeptide comprises an amino acid sequence as set forth in SEQ. ID NO: 181 (AI590292_P6).

According to one embodiment, the isolated polypeptide is a chimeric polypeptide comprising a first amino acid sequence being at least about 70%, optionally at least about 80%, preferably at least about 85%, more preferably at least about 90% and most preferably at least about 95%, homologous to a polypeptide having the sequence ADPQRLHRDCAPLPLALPRCCSSRPAALR (SEQ ID NO:390) corresponding to amino acids 1-29 of AI590292_P12 (SEQ ID NO:182), a second amino acid sequence being at least about 90% homologous to amino acids 1-91 of PODO_HUMAN (SEQ ID NO:178), which also corresponds to amino acids 30-120 of AI590292_P12 (SEQ ID NO:182), a third amino acid sequence being at least about 90% homologous to amino acids 151-291 of PODO_HUMAN (SEQ ID NO:178), which also corresponds to amino acids 121-261 of AI590292_P12 (SEQ ID NO:182), and a fourth amino acid sequence being at least about 70%, optionally at least about 80%, preferably at least about 85%, more preferably at least about 90% and most preferably at least about 95% homologous to a polypeptide having the sequence LKTLQKEE (SEQ ID NO:388) corresponding to amino acids 262-269 of AI590292_P12 (SEQ ID NO:182), wherein said first amino acid sequence, second amino acid sequence, third amino acid sequence and fourth amino acid sequence are contiguous and in a sequential order.

According to one embodiment, the present invention provides an isolated polypeptide comprising a head of AI590292_P12 (SEQ ID NO:182), comprising a polypeptide being at least about 70%, optionally at least about 80%, preferably at least about 85%, more preferably at least about 90% and most preferably at least about 95% homologous to the sequence ADPQRLHRDCAPLPLALPRCCSSRPAALR (SEQ ID NO:390) of AI590292_P12 (SEQ ID NO:182).

According to one embodiment, the present invention provides an isolated polypeptide comprising an edge portion of AI590292_P12 (SEQ ID NO:182), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise EG, having a structure as follows: a sequence starting from any of amino acid numbers 120-x to 120; and ending at any of amino acid numbers 121+((n−2)−x), in which x varies from 0 to n−2.

According to one embodiment, the present invention provides an isolated polypeptide comprising an edge portion of AI590292_P12 (SEQ ID NO:182), comprising an amino acid sequence being at least about 70%, optionally at least about 80%, preferably at least about 85%, more preferably at least about 90% and most preferably at least about 95% homologous to the sequence LKTLQKEE (SEQ ID NO:388) of AI590292_P12 (SEQ ID NO:182).

According to one embodiment, the isolated polypeptide is a chimeric polypeptide comprising a first amino acid sequence being at least about 70%, optionally at least about 80%, preferably at least about 85%, more preferably at least about 90% and most preferably at least about 95%, homologous to a polypeptide having the sequence ADPQRLHRDCAPLPLALPRCCSSRPAALR (SEQ ID NO:390) corresponding to amino acids 1-29 of AI590292_P12 (SEQ ID NO:182), a second amino acid sequence being at least about 90% homologous to amino acids 1-91 of Q9NP85-2 (SEQ ID NO:179), which also corresponds to amino acids 30-120 of AI590292_P12 (SEQ ID NO:182), a third amino acid sequence being at least about 90% homologous to amino acids 151-178 of Q9NP85-2 (SEQ ID NO:179), which also corresponds to amino acids 121-148 of AI590292_P12 (SEQ ID NO:182), a fourth amino acid sequence being at least about 70%, optionally at least about 80%, preferably at least about 85%, more preferably at least about 90% and most preferably at least about 95% homologous to a polypeptide having the sequence IVTKDMFIMEIDAICYYRMENASLLLSSLAHVSKAVQFLVQTTMKRLLAHRSLTEILLER KSIAQDAK (SEQ ID NO:387) corresponding to amino acids 149-216 of AI590292_P12 (SEQ ID NO:182), a fifth amino acid sequence being at least about 90% homologous to amino acids 179-223 of Q9NP85-2 (SEQ ID NO:179), which also corresponds to amino acids 217-261 of AI590292_P12 (SEQ ID NO:182), and a sixth amino acid sequence being at least about 70%, optionally at least about 80%, preferably at least about 85%, more preferably at least about 90% and most preferably at least about 95% homologous to a polypeptide having the sequence LKTLQKEE (SEQ ID NO:388) corresponding to amino acids 262-269 of AI590292_P12 (SEQ ID NO:182), wherein said first amino acid sequence, second amino acid sequence, third amino acid sequence, fourth amino acid sequence, fifth amino acid sequence and sixth amino acid sequence are contiguous and in a sequential order.

According to one embodiment, the present invention provides an isolated polypeptide comprising a head of AI590292_P12 (SEQ ID NO:182), comprising a polypeptide being at least about 70%, optionally at least about 80%, preferably at least about 85%, more preferably at least about 90% and most preferably at least about 95% homologous to the sequence ADPQRLHRDCAPLPLALPRCCSSRPAALR (SEQ ID NO:390) of AI590292_P12 (SEQ ID NO:182).

According to one embodiment, the present invention provides an isolated polypeptide comprising an edge portion of AI590292_P12 (SEQ ID NO:182), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise EG, having a structure as follows: a sequence starting from any of amino acid numbers 120-x to 120; and ending at any of amino acid numbers 121+((n−2)−x), in which x varies from 0 to n−2.

According to one embodiment, the present invention provides an isolated polypeptide comprising an edge portion of AI590292_P12 (SEQ ID NO:182), comprising an amino acid sequence being at least about 70%, optionally at least about 80%, preferably at least about 85%, more preferably at least about 90% and most preferably at least about 95% homologous to the sequence IVTKDMFIMEIDAICYYRMENASLLLSSLAHVSKAVQFLVQTTMKRLLAHRSLTEILLER KSIAQDAK (SEQ ID NO:387) of AI590292_P12 (SEQ ID NO:182).

According to one embodiment, the present invention provides an isolated polypeptide comprising an edge portion of AI590292_P12 (SEQ ID NO:182), comprising an amino acid sequence being at least about 70%, optionally at least about 80%, preferably at least about 85%, more preferably at least about 90% and most preferably at least about 95% homologous to the sequence LKTLQKEE (SEQ ID NO:388) of AI590292_P12 (SEQ ID NO:182).

According to another embodiment, the isolated polypeptide comprises an amino acid sequence as set forth in SEQ. ID NO: 182 (AI590292_P12).

According to one embodiment, the isolated polypeptide is a chimeric polypeptide comprising a first amino acid sequence being at least about 90% homologous to amino acids 1-92 of PODO_HUMAN (SEQ ID NO:178), which also corresponds to amino acids 1-92 of AI590292_P13 (SEQ ID NO:183), and a second amino acid sequence being at least about 70%, optionally at least about 80%, preferably at least about 85%, more preferably at least about 90% and most preferably at least about 95% homologous to a polypeptide having the sequence CTRV (SEQ ID NO:393) corresponding to amino acids 93-96 of AI590292_P13 (SEQ ID NO:183), wherein said first amino acid sequence and second amino acid sequence are contiguous and in a sequential order.

According to one embodiment, the present invention provides an isolated polypeptide comprising an edge portion of AI590292_P13 (SEQ ID NO:183), comprising an amino acid sequence being at least about 70%, optionally at least about 80%, preferably at least about 85%, more preferably at least about 90% and most preferably at least about 95% homologous to the sequence CTRV (SEQ ID NO:393) of AI590292_P13 (SEQ ID NO:183).

According to another embodiment, the isolated polypeptide comprises an amino acid sequence as set forth in SEQ. ID NO: 183 (AI590292_P13).

According to certain embodiments, the present invention now discloses a novel cluster designated herein HUMUMOD, comprising novel amino acid and nucleic acid sequences that are variants of the known protein Uromodulin precursor (SEQ ID NO:242) (SwissProt accession identifier UROM_HUMAN (SEQ ID NO:242); known also according to the synonyms Tamm-Horsfall urinary glycoprotein; THP). The novel variant polynucleotides and polypeptides described by the present invention are useful as diagnostic markers, preferably as serum markers.

The present invention now discloses that HUMUMOD variants are specifically expressed in kidney tissues, and thus can be indicative of the presence, onset, severity or prognosis and/or staging and or progression of renal disease and/or condition, as described in a greater detail below.

According to one embodiment, the present invention provides an isolated polypeptide comprising an edge portion of HUMUMOD_P6 (SEQ ID NO:246), comprising an amino acid sequence being at least about 70%, optionally at least about 80%, preferably at least about 85%, more preferably at least about 90% and most preferably at least about 95% homologous to the sequence APEHKPGPVSSPPISSGLMTSPPISSVLRARHKPQISHRYLPPGAQAGMWGQ (SEQ ID NO:394) of HUMUMOD_P6 (SEQ ID NO:246).

According to one embodiment, the present invention provides an isolated polypeptide comprising an edge portion of HUMUMOD_P6 (SEQ ID NO:246), comprising an amino acid sequence being at least about 70%, optionally at least about 80%, preferably at least about 85%, more preferably at least about 90% and most preferably at least about 95% homologous to the sequence FVGQGGARMAETCVPVLRCNTAAPMWLNGT (SEQ ID NO:395) of HUMUMOD_P6 (SEQ ID NO:246).

According to one embodiment, the present invention provides an isolated polypeptide comprising an edge portion of HUMUMOD_P6 (SEQ ID NO:246), comprising an amino acid sequence being at least about 70%, optionally at least about 80%, preferably at least about 85%, more preferably at least about 90% and most preferably at least about 95% homologous to the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) of HUMUMOD_P6 (SEQ ID NO:246).

According to one embodiment, the isolated polypeptide is a chimeric polypeptide comprising a first amino acid sequence being at least about 90% homologous to amino acids 1-324 of UROM_HUMAN (SEQ ID NO:242), which also corresponds to amino acids 1-324 of HUMUMOD_P6 (SEQ ID NO:246), and a second amino acid sequence being at least about 70%, optionally at least about 80%, preferably at least about 85%, more preferably at least about 90% and most preferably at least about 95% homologous to a polypeptide having the sequence APEHKPGPVSSPPISSGLMTSPPISSVLRARHKPQISHRYLPPGAQAGMWGQ (SEQ ID NO:394) corresponding to amino acids 325-376 of HUMUMOD_P6 (SEQ ID NO:246), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-204 of Q8IYG0_HUMAN (SEQ ID NO:244), which also corresponds to amino acids 1-204 of HUMUMOD_P6 (SEQ ID NO:246), a second amino acid sequence being at least about 70%, optionally at least about 80%, preferably at least about 85%, more preferably at least about 90% and most preferably at least about 95% homologous to a polypeptide having the sequence FVGQGGARMAETCVPVLRCNTAAPMWLNGT (SEQ ID NO:398) corresponding to amino acids 205-234 of HUMUMOD_P6 (SEQ ID NO:246), a third amino acid sequence being at least about 90% homologous to amino acids 206-295 of Q8IYG0_HUMAN (SEQ ID NO:244), which also corresponds to amino acids 235-324 of HUMUMOD_P6 (SEQ ID NO:246), and a fourth amino acid sequence being at least about 70%, optionally at least about 80%, preferably at least about 85%, more preferably at least about 90% and most preferably at least about 95% homologous to a polypeptide having the sequence APEHYKPGPVSSPPISSGLMTSPPISSVLRARHKPQISHRYLPPGAQAGMWGQ (SEQ ID NO:394) corresponding to amino acids 325-376 of HUMUMOD_P6 (SEQ ID NO:246), wherein said first amino acid sequence, second amino acid sequence, third amino acid sequence and fourth amino acid sequence are contiguous and in a sequential order.

According to one embodiment, the isolated polypeptide is a chimeric polypeptide comprising a first amino acid sequence being at least about 90% homologous to amino acids 1-65 of Q6ZS84_HUMAN (SEQ ID NO:245), which also corresponds to amino acids 1-65 of HUMUMOD_P6 (SEQ ID NO:246), a second amino acid sequence being at least about 70%, optionally at least about 80%, preferably at least about 85%, more preferably at least about 90% and most preferably at least about 95% homologous to a polypeptide having the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) corresponding to amino acids 66-198 of HUMUMOD_P6 (SEQ ID NO:246), a third amino acid sequence being at least about 90% homologous to amino acids 66-191 of Q6ZS84_HUMAN (SEQ ID NO:245), which also corresponds to amino acids 199-324 of HUMUMOD_P6 (SEQ ID NO:246), and a fourth amino acid sequence being at least about 70%, optionally at least about 80%, preferably at least about 85%, more preferably at least about 90% and most preferably at least about 95% homologous to a polypeptide having the sequence APEHKPGPVSSPPISSGLMTSPPISSVLRARHKPQISHRYLPPGAQAGMWGQ (SEQ ID NO:394) corresponding to amino acids 325-376 of HUMUMOD_P6 (SEQ ID NO:246), wherein said first amino acid sequence, second amino acid sequence, third amino acid sequence and fourth amino acid sequence are contiguous and in a sequential order.

According to another embodiment, the isolated polypeptide comprises an amino acid sequence as set forth in SEQ. ID NO: 246 (HUMUMOD_P6).

According to one embodiment, the present invention provides an isolated polypeptide comprising an edge portion of HUMUMOD_P7 (SEQ ID NO:247), comprising an amino acid sequence being at least about 70%, optionally at least about 80%, preferably at least about 85%, more preferably at least about 90% and most preferably at least about 95% homologous to the sequence QTQERMLGTDEGAQLTWRKKSAHCKLLAGTSPGLAVSWSNVAPSSAC (SEQ ID NO:397) of HUMUMOD_P7 (SEQ ID NO:247).

According to one embodiment, the present invention provides an isolated polypeptide comprising an edge portion of HUMUMOD_P7 (SEQ ID NO:247), comprising an amino acid sequence being at least about 70%, optionally at least about 80%, preferably at least about 85%, more preferably at least about 90% and most preferably at least about 95% homologous to the sequence FVGQGGARMAETCVPVLRCNTAAPMWLNGT (SEQ ID NO:398) of HUMUMOD_P7 (SEQ ID NO:247).

According to one embodiment, the present invention provides an isolated polypeptide comprising an edge portion of HUMUMOD_P7 (SEQ ID NO:247), comprising an amino acid sequence being at least about 70%, optionally at least about 80%, preferably at least about 85%, more preferably at least about 90% and most preferably at least about 95% homologous to the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:399) of HUMUMOD_P7 (SEQ ID NO:247).

According to one embodiment, the isolated polypeptide is a chimeric polypeptide comprising a first amino acid sequence being at least about 90% homologous to amino acids 1-444 of UROM_HUMAN (SEQ ID NO:242), which also corresponds to amino acids 1-444 of HUMUMOD_P7 (SEQ ID NO:247), a second amino acid sequence being at least about 70%, optionally at least about 80%, preferably at least about 85%, more preferably at least about 90% and most preferably at least about 95% homologous to a polypeptide having the sequence QTQERMLGTDEGAQLTWRKKSAHCKLLAGTSPGLAVSWSNVAPSSAC (SEQ ID NO:397) corresponding to amino acids 445-491 of HUMUMOD_P7 (SEQ ID NO:247), and a third amino acid sequence being at least about 90% homologous to amino acids 445-640 of UROM_HUMAN (SEQ ID NO:242), which also corresponds to amino acids 492-687 of HUMUMOD_P7 (SEQ ID NO:247), wherein said first amino acid sequence, second amino acid sequence and third amino acid sequence are contiguous and in a sequential order.

According to one embodiment, the isolated polypeptide is a chimeric polypeptide comprising a first amino acid sequence being at least about 90% homologous to amino acids 1-204 of Q8IYG0_HUMAN (SEQ ID NO:244), which also corresponds to amino acids 1-204 of HUMUMOD_P7 (SEQ ID NO:247), a second amino acid sequence being at least about 70%, optionally at least about 80%, preferably at least about 85%, more preferably at least about 90% and most preferably at least about 95% homologous to a polypeptide having the sequence FVGQGGARMAETCVPVLRCNTAAPMWLNGT (SEQ ID NO:400) corresponding to amino acids 205-234 of HUMUMOD_P7 (SEQ ID NO:247), a third amino acid sequence being at least about 90% homologous to amino acids 206-415 of Q8IYG0_HUMAN (SEQ ID NO:244), which also corresponds to amino acids 235-444 of HUMUMOD_P7 (SEQ ID NO:247), a fourth amino acid sequence being at least about 70%, optionally at least about 80%, preferably at least about 85%, more preferably at least about 90% and most preferably at least about 95% homologous to a polypeptide having the sequence QTQERMLGTDEGAQLTWRKKSAHCKLLAGTSPGLAVSWSNVAPSSAC (SEQ ID NO:397) corresponding to amino acids 445-491 of HUMUMOD_P7 (SEQ ID NO:247), and a fifth amino acid sequence being at least about 90% homologous to amino acids 416-611 of Q8IYG0_HUMAN (SEQ ID NO:244), which also corresponds to amino acids 492-687 of HUMUMOD_P7 (SEQ ID NO:247), wherein said first amino acid sequence, second amino acid sequence, third amino acid sequence, fourth amino acid sequence and fifth amino acid sequence are contiguous and in a sequential order.

According to one embodiment, the isolated polypeptide is a chimeric polypeptide comprising a first amino acid sequence being at least about 90% homologous to amino acids 1-65 of Q6ZS84_HUMAN (SEQ ID NO:245), which also corresponds to amino acids 1-65 of HUMUMOD_P7 (SEQ ID NO:247), a second amino acid sequence being at least about 70%, optionally at least about 80%, preferably at least about 85%, more preferably at least about 90% and most preferably at least about 95% homologous to a polypeptide having the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:401) corresponding to amino acids 66-198 of HUMUMOD_P7 (SEQ ID NO:247), a third amino acid sequence being at least about 90% homologous to amino acids 66-311 of Q6ZS84_HUMAN (SEQ ID NO:245), which also corresponds to amino acids 199-444 of HUMUMOD_P7 (SEQ ID NO:247), a fourth amino acid sequence being at least about 70%, optionally at least about 80%, preferably at least about 85%, more preferably at least about 90% and most preferably at least about 95% homologous to a polypeptide having the sequence QTQERMLGTDEGAQLTWRKKSAHCKLLAGTSPGLAVSWSNVAPSSAC (SEQ ID NO:397) corresponding to amino acids 445′-491 of HUMUMOD_P7 (SEQ ID NO:247), and a fifth amino acid sequence being at least about 90% homologous to amino acids 312-507 of Q6ZS84_HUMAN (SEQ ID NO:245), which also corresponds to amino acids 492-687 of HUMUMOD_P7 (SEQ ID NO:247), wherein said first amino acid sequence, second amino acid sequence, third amino acid sequence, fourth amino acid sequence and fifth amino acid sequence are contiguous and in a sequential order.

According to another embodiment, the isolated polypeptide comprises an amino acid sequence as set forth in SEQ. ID NO: 247 (HUMUMOD_P7).

According to one embodiment, the isolated polypeptide is a chimeric polypeptide comprising a first amino acid sequence being at least about 90% homologous to amino acids 1-443 of UROM_HUMAN (SEQ ID NO:242), which also corresponds to amino acids 1-443 of HUMUMOD_P14 (SEQ ID NO:248), and a second amino acid sequence being at least about 90% homologous to amino acids 526-640 of UROM_HUMAN (SEQ ID NO:242), which also corresponds to amino acids 444-558 of HUMUMOD_P14 (SEQ ID NO:248), wherein said first amino acid sequence and second amino acid sequence are contiguous and in a sequential order.

According to one embodiment, the present invention provides an isolated polypeptide comprising an edge portion of HUMUMOD_P14 (SEQ ID NO:248), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise VR, having a structure as follows: a sequence starting from any of amino acid numbers 443-x to 443; and ending at any of amino acid numbers 444+((n−2)−x), in which x varies from 0 to n−2.

According to one embodiment, the isolated polypeptide is a chimeric polypeptide comprising a first amino acid sequence being at least about 90% homologous to amino acids 1-204 of Q8IYG0_HUMAN (SEQ ID NO:244), which also corresponds to amino acids 1-204 of HUMUMOD_P14 (SEQ ID NO:248), a second amino acid sequence being at least about 70%, optionally at least about 80%, preferably at least about 85%, more preferably at least about 90% and most preferably at least about 95% homologous to a polypeptide having the sequence FVGQGGARMAETCVPVLRCNTAAPMWLNGT (SEQ ID NO:395) corresponding to amino acids 205-234 of HUMUMOD_P14 (SEQ ID NO:248), a third amino acid sequence being at least about 90% homologous to amino acids 206-414 of Q8IYG0_HUMAN (SEQ ID NO:244), which also corresponds to amino acids 235-443 of HUMUMOD_P14 (SEQ ID NO:248), and a fourth amino acid sequence being at least about 90% homologous to amino acids 497-611 of Q8IYG0_HUMAN (SEQ ID NO:244), which also corresponds to amino acids 444-558 of HUMUMOD_P14 (SEQ ID NO:248), wherein said first amino acid sequence, second amino acid sequence, third amino acid sequence and fourth amino acid sequence are contiguous and in a sequential order.

According to one embodiment, the present invention provides an isolated polypeptide comprising an edge portion of HUMUMOD_P14 (SEQ ID NO:248), comprising an amino acid sequence being at least about 70%, optionally at least about 80%, preferably at least about 85%, more preferably at least about 90% and most preferably at least about 95% homologous to the sequence FVGQGGARMAETCVPVLRCNTAAPMWLNGT (SEQ ID NO:395) of HUMUMOD_P14 (SEQ ID NO:248).

According to one embodiment, the isolated polypeptide is a chimeric polypeptide comprising a first amino acid sequence being at least about 90% homologous to amino acids 1-65 of Q6ZS84_HUMAN (SEQ ID NO:245), which also corresponds to amino acids 1-65 of HUMUMOD_P14 (SEQ ID NO:248), a second amino acid sequence being at least about 70%, optionally at least about 80%, preferably at least about 85%, more preferably at least about 90% and most preferably at least about 95% homologous to a polypeptide having the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) corresponding to amino acids 66-198 of HUMUMOD_P14 (SEQ ID NO:248), a third amino acid sequence being at least about 90% homologous to amino acids 66-310 of Q6ZS84_HUMAN (SEQ ID NO:245), which also corresponds to amino acids 199-443 of HUMUMOD_P14 (SEQ ID NO:248), and a fourth amino acid sequence being at least about 90% homologous to amino acids 393-507 of Q6ZS84_HUMAN (SEQ ID NO:245), which also corresponds to amino acids 444-558 of HUMUMOD_P14 (SEQ ID NO:248), wherein said first amino acid sequence, second amino acid sequence, third amino acid sequence and fourth amino acid sequence are contiguous and in a sequential order.

According to one embodiment, the present invention provides an isolated polypeptide comprising an edge portion of HUMUMOD_P14 (SEQ ID NO:248), comprising an amino acid sequence being at least about 70%, optionally at least about 80%, preferably at least about 85%, more preferably at least about 90% and most preferably at least about 95% homologous to the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) of HUMUMOD_P14 (SEQ ID NO:248).

According to one embodiment, the present invention provides an isolated polypeptide comprising an edge portion of HUMUMOD_P24 (SEQ ID NO:249), comprising an amino acid sequence being at least about 70%, optionally at least about 80%, preferably at least about 85%, more preferably at least about 90% and most preferably at least about 95% homologous to the sequence RCGQRGSLGPLDGSNPKPLNHELPCQLPPTGSWE (SEQ ID NO:402) of HUMUMOD_P24 (SEQ ID NO:249).

According to one embodiment, the present invention provides an isolated polypeptide comprising an edge portion of HUMUMOD_P24 (SEQ ID NO:249), comprising an amino acid sequence being at least about 70%, optionally at least about 80%, preferably at least about 85%, more preferably at least about 90% and most preferably at least about 95% homologous to the sequence FVGQGGARMAETCVPVLRCNTAAPMWLNGT (SEQ ID NO:403) of HUMUMOD_P24 (SEQ ID NO:249).

According to one embodiment, the present invention provides an isolated polypeptide comprising an edge portion of HUMUMOD_P24 (SEQ ID NO:249), comprising an amino acid sequence being at least about 70%, optionally at least about 80%, preferably at least about 85%, more preferably at least about 90% and most preferably at least about 95% homologous to the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:404) of HUMUMOD_P24 (SEQ ID NO:249).

According to one embodiment, the isolated polypeptide is a chimeric polypeptide comprising a first amino acid sequence being at least about 90% homologous to amino acids 1-443 of UROM_HUMAN (SEQ ID NO:242), which also corresponds to amino acids 1-443 of HUMUMOD_P24 (SEQ ID NO:249), and a second amino acid sequence being at least about 70%, optionally at least about 80%, preferably at least about 85%, more preferably at least about 90% and most preferably at least about 95% homologous to a polypeptide having the sequence RCGQRGSLGPLDGSNPKPLNHELPCQLPPTGSWE corresponding to amino acids 444-477 of HUMUMOD_P24 (SEQ ID NO:249), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-204 of Q8IYG0_HUMAN (SEQ ID NO:244), which also corresponds to amino acids 1-204 of HUMUMOD_P24 (SEQ ID NO:249), a second amino acid sequence being at least about 70%, optionally at least about 80%, preferably at least about 85%, more preferably at least about 90% and most preferably at least about 95% homologous to a polypeptide having the sequence FVGQGGARMAETCVPVLRCNTAAPMWLNGT (SEQ ID NO:395) corresponding to amino acids 205-234 of HUMUMOD_P24 (SEQ ID NO:249), a third amino acid sequence being at least about 90% homologous to amino acids 206-414 of Q8IYG0_HUMAN (SEQ ID NO:244), which also corresponds to amino acids 235-443 of HUMUMOD_P24 (SEQ ID NO:249), and a fourth amino acid sequence being at least about 70%, optionally at least about 80%, preferably at least about 85%, more preferably at least about 90% and most preferably at least about 95% homologous to a polypeptide having the sequence RCGQRGSLGPLDGSNPKPLNHELPCQLPPTGSWE (SEQ ID NO:402) corresponding to amino acids 444-477 of HUMUMOD_P24 (SEQ ID NO:249), wherein said first amino acid sequence, second amino acid sequence, third amino acid sequence and fourth amino acid sequence are contiguous and in a sequential order.

According to one embodiment, the isolated polypeptide is a chimeric polypeptide comprising a first amino acid sequence being at least about 90% homologous to amino acids 1-65 of Q6ZS84_HUMAN (SEQ ID NO:245), which also corresponds to amino acids 1-65 of HUMUMOD_P24 (SEQ ID NO:249), a second amino acid sequence being at least about 70%, optionally at least about 80%, preferably at least about 85%, more preferably at least about 90% and most preferably at least about 95% homologous to a polypeptide having the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) corresponding to amino acids 66-198 of HUMUMOD_P24 (SEQ ID NO:249), a third amino acid sequence being at least about 90% homologous to amino acids 66-310 of Q6ZS84_HUMAN (SEQ ID NO:245), which also corresponds to amino acids 199-443 of HUMUMOD_P24 (SEQ ID NO:249), and a fourth amino acid sequence being at least about 70%, optionally at least about 80%, preferably at least about 85%, more preferably at least about 90% and most preferably at least about 95% homologous to a polypeptide having the sequence amino acids 444-477 of HUMUMOD_P24 (SEQ ID NO:249), wherein said first amino acid sequence, second amino acid sequence, third amino acid sequence and fourth amino acid sequence are contiguous and in a sequential order.

According to certain embodiments, the present invention now discloses a novel cluster designated herein HSCP2, comprising novel amino acid and nucleic acid sequences that are variants of the known protein Ceruloplasmin precursor (SEQ ID NO:308) (SwissProt accession identifier CERU_HUMAN (SEQ ID NO:308); known also according to the synonyms EC 1.16.3.1; Ferroxidase). The novel variant polynucleotides and polypeptides described by the present invention are useful as diagnostic markers, preferably as serum markers.

The present invention now discloses that HSCP2 variants are overexpressed in cancerous tissues, particularly in cancerous lung tissues and cancerous ovarian tissues, and thus can be used for the diagnosis, prognosis, treatment selection, and treatment monitoring and/or assessment of cancers, particularly lung cancer and ovarian cancer, as is described in a greater detail below.

According to one embodiment, the present invention provides an isolated polypeptide comprising an edge portion of HSCP2_P3 (SEQ ID NO:310), comprising an amino acid sequence being at least about 70%, optionally at least about 80%, preferably at least about 85%, more preferably at least about 90% and most preferably at least about 95% homologous to the sequence GGTSM (SEQ ID NO:405) of HSCP2_P3 (SEQ ID NO:310).

According to one embodiment, the present invention provides an isolated polypeptide comprising an edge portion of HSCP2_P3 (SEQ ID NO:310), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAGVQTESSTVTPTLPGETLT YVWKIPERSGAGTEDSACIPWAYYSTVDQVKDLYSGLIGPLIvCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKHRGVYSSDVFDIFPGTYQTLEMFPRTP GIWLLHCHVTDHIHAGMETTYTVLQNEGGTSM (SEQ ID NO:406) of HSCP2_P3 (SEQ ID NO:310).

According to one embodiment, the isolated polypeptide is a chimeric polypeptide comprising a first amino acid sequence being at least about 90% homologous to amino acids 1-1060 of CERU_HUMAN (SEQ ID NO:308), which also corresponds to amino acids 1-1060 of HSCP2_P3 (SEQ ID NO:310), and a second amino acid sequence being at least about 70%, optionally at least about 80%, preferably at least about 85%, more preferably at least about 90% and most preferably at least about 95% homologous to a polypeptide having the sequence GGTSM (SEQ ID NO:405) corresponding to amino acids 1061-1065 of HSCP2_P3 (SEQ ID NO:310), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P3 (SEQ ID NO:310), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKHRGVYSSDVFDIFPGTYQTLEMFPRTP GIWLLHCHVTDHIHAGMETTYTVLQNEGGTSM (SEQ ID NO:406) corresponding to amino acids 208-1065 of HSCP2_P3 (SEQ ID NO:310), wherein said first amino acid sequence and second amino acid sequence are contiguous and in a sequential order.

According to one embodiment, the present invention provides an isolated polypeptide comprising an edge portion of HSCP2_P4 (SEQ ID NO:311), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQK (SEQ ID NO:407) of HSCP2_P4 (SEQ ID NO:311).

According to one embodiment, the isolated polypeptide is a chimeric polypeptide comprising a first amino acid sequence being at least about 90% homologous to amino acids 1-761 of CERU_HUMAN (SEQ ID NO:308), which also corresponds to amino acids 1-761 of HSCP2_P4 (SEQ ID NO:311), and a second amino acid K corresponding to amino acid 762 of HSCP2_P4 (SEQ ID NO:311), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P4 (SEQ ID NO:311), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNBLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQK (SEQ ID NO:407) corresponding to amino acids 208-762 of HSCP2_P4 (SEQ ID NO:311), wherein said first amino acid sequence and second amino acid sequence are contiguous and in a sequential order.

According to one embodiment, the present invention provides an isolated polypeptide comprising an edge portion of HSCP2_P5 (SEQ ID NO:312), comprising an amino acid sequence being at least about 70%, optionally at least about 80%, preferably at least about 85%, more preferably at least about 90% and most preferably at least about 95% homologous to the sequence GEYP (SEQ ID NO:408) of HSCP2P5 (SEQ ID NO:312).

According to one embodiment, the present invention provides an isolated polypeptide comprising an edge portion of HSCP2_P5 (SEQ ID NO:312), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKHRGVYSSDVFDIFPGTYQTLEMFPRTP GIWLLHCHVTDHIHAGMETTYTVLQNEGEYPDTKSG (SEQ ID NO:409) of HSCP2_P5 (SEQ ID NO:312).

According to one embodiment, the isolated polypeptide is a chimeric polypeptide comprising a first amino acid sequence being at least about 90% homologous to amino acids 1-1060 of CERU_HUMAN (SEQ ID NO:308), which also corresponds to amino acids 1-1060 of HSCP2_P5 (SEQ ID NO:312), a second amino acid sequence being at least about 70%, optionally at least about 80%, preferably at least about 85%, more preferably at least about 90% and most preferably at least about 95% homologous to a polypeptide having the sequence GEYP (SEQ ID NO:408) corresponding to amino acids 1061-1064 of HSCP2_P5 (SEQ ID NO:312), and a third amino acid sequence being at least about 90% homologous to DTKSG corresponding to amino acids 1061-1065 of CERU_HUMAN (SEQ ID NO:308), which also corresponds to amino acids 1065-1069 of HSCP2_P5 (SEQ ID NO:312), wherein said first amino acid sequence, second amino acid sequence and third amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P5 (SEQ ID NO:312), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHTYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKHRGVYSSDVFDIFPGTYQTLEMFPRTP GIWLLHCHVTDHIHAGMETTYTVLQNEGEYPDTKSG (SEQ ID NO:409) corresponding to amino acids 208-1069 of HSCP2_P5 (SEQ ID NO:312), wherein said first amino acid sequence and second amino acid sequence are contiguous and in a sequential order.

According to one embodiment, the present invention provides an isolated polypeptide comprising an edge portion of HSCP2_P6 (SEQ ID NO:313), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKGSL (SEQ ID NO:410) of HSCP2_P6 (SEQ ID NO:313).

According to one embodiment, the present invention provides an isolated polypeptide comprising an edge portion of HSCP2_P6 (SEQ ID NO:313), comprising an amino acid sequence being at least about 70%, optionally at least about 80%, preferably at least about 85%, more preferably at least about 90% and most preferably at least about 95% homologous to the sequence GSL (SEQ ID NO:411) of HSCP2_P6 (SEQ ID NO:313).

According to one embodiment, the isolated polypeptide is a chimeric polypeptide comprising a first amino acid sequence being at least about 90% homologous to amino acids 1-1006 of CERU_HUMAN (SEQ ID NO:308), which also corresponds to amino acids 1-1006 of HSCP2_P6 (SEQ ID NO:313), and a second amino acid sequence being at least about 70%, optionally at least about 80%, preferably at least about 85%, more preferably at least about 90% and most preferably at least about 95% homologous to a polypeptide having the sequence GSL (SEQ ID NO:411) corresponding to amino acids 1007-1009 of HSCP2_P6 (SEQ ID NO:313), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P6 (SEQ ID NO:313), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKGSL (SEQ ID NO:410) corresponding to amino acids 208-1009 of HSCP2_P6 (SEQ ID NO:313), wherein said first amino acid sequence and second amino acid sequence are contiguous and in a sequential order.

According to one embodiment, the present invention provides an isolated polypeptide comprising an edge portion of HSCP2_P8 (SEQ ID NO:314), comprising an amino acid sequence being at least about 70%, optionally at least about 80%, preferably at least about 85%, more preferably at least about 90% and most preferably at least about 95% homologous to the sequence KCFQEHLEFGYSTAM (SEQ ID NO:412) of HSCP2_P8 (SEQ ID NO:314).

According to one embodiment, the present invention provides an isolated polypeptide comprising an edge portion of HSCP2_P8 (SEQ ID NO:314), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKKCFQEHLEFGYSTAM (SEQ ID NO:413) of HSCP2_P8 (SEQ ID NO:314).

According to one embodiment, the isolated polypeptide is a chimeric polypeptide comprising a first amino acid sequence being at least about 90% homologous to amino acids 1-1006 of CERU_HUMAN (SEQ ID NO:308), which also corresponds to amino acids 1-1006 of HSCP2_P8 (SEQ ID NO:314), and a second amino acid sequence being at least about 70%, optionally at least about 80%, preferably at least about 85%, more preferably at least about 90% and most preferably at least about 95% homologous to a polypeptide having the sequence KCFQEHLEFGYSTAM (SEQ ID NO:412) corresponding to amino acids 1007-1021 of HSCP2_P8 (SEQ ID NO:314), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P8 (SEQ ID NO:314), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKKCFQEHLEFGYSTAM (SEQ ID NO:413) corresponding to amino acids 208-1021 of HSCP2_P8 (SEQ ID NO:314), wherein said first amino acid sequence and second amino acid sequence are contiguous and in a sequential order.

According to one embodiment, the present invention provides an isolated polypeptide comprising an edge portion of HSCP2_P15 (SEQ ID NO:315), comprising an amino acid sequence being at least about 70%, optionally at least about 80%, preferably at least about 85%, more preferably at least about 90% and most preferably at least about 95% homologous to the sequence VRAIHGNYSCSV (SEQ ID NO:414) of HSCP2_P15 (SEQ ID NO:315).

According to one embodiment, the present invention provides an isolated polypeptide comprising an edge portion of HSCP2_P15 (SEQ ID NO:315), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKVRAIHGNYSCSV (SEQ ID NO:415) of HSCP2_P15 (SEQ ID NO:315).

According to one embodiment, the isolated polypeptide is a chimeric polypeptide comprising a first amino acid sequence being at least about 90% homologous to amino acids 1-1006 of CERU_HUMAN (SEQ ID NO:308), which also corresponds to amino acids 1-1006 of HSCP2_P15 (SEQ ID NO:315), and a second amino acid sequence being at least about 70%, optionally at least about 80%, preferably at least about 85%, more preferably at least about 90% and most preferably at least about 95% homologous to a polypeptide having the sequence VRAIHGNYSCSV (SEQ ID NO:414) corresponding to amino acids 1007-1018 of HSCP2_P15 (SEQ ID NO:315), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P15 (SEQ ID NO:315), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKVRAIHGNYSCSV (SEQ ID NO:415) corresponding to amino acids 208-1018 of HSCP2_P15 (SEQ ID NO:315), wherein said first amino acid sequence and second amino acid sequence are contiguous and in a sequential order.

According to another embodiment, the isolated polypeptide comprises an amino acid sequence as set forth in SEQ. ID NO: 315 (HSCP2_P15).

According to one embodiment, the isolated polypeptide is a chimeric polypeptide comprising a first amino acid sequence being at least about 90% homologous to amino acids 1-621 of CERU_HUMAN (SEQ ID NO:308), which also corresponds to amino acids 1-621 of HSCP2_P16 (SEQ ID NO:316), a second bridging amino acid sequence comprising of W, and a third amino acid sequence being at least about 90% homologous to amino acids 694-1065 of CERU_HUMAN (SEQ ID NO:308), which also corresponds to amino acids 623-994 of HSCP2_P16 (SEQ ID NO:316), wherein said first amino acid sequence, second amino acid sequence and third amino acid sequence are contiguous and in a sequential order.

According to one embodiment, the present invention provides an isolated polypeptide comprising an edge portion of HSCP2_P16 (SEQ ID NO:316), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about 3 amino acids comprise HWT having a structure as follows (numbering according to HSCP2_P16 (SEQ ID NO:316): a sequence starting from any of amino acid numbers 621-x to 621; and ending at any of amino acid numbers 623+((n−3)−x), in which x varies from 0 to n−3.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P16 (SEQ ID NO:316), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMH WTFNVECLTTDHYTGGMKQKYTVNQCRRQSEDSTFYLGERTYYIAAVEVEWDYSPQRE WEKELHHLQEQNVSNAFLDKGEFYIGSKYKKVVYRQYTDSTFRVPVERKAEEEHLGILG PQLHADVGDKVKIIFKNMATRPYSIHAHGVQTESSTVTPTLPGETLTYVWKIPERSGAGT EDSACIPWAYYSTVDQVKDLYSGLIGPLIVCRRPYLKVFNPRRKLEFALLFLVFDENESW YLDDNIKTYSDHPEKVNKDDEEFIESNKMHAINGRMFGNLQGLTMHVGDEVNWYLMG MGNEIDLHTVHFHGHSFQYKHRGVYSSDVFDIFPGTYQTLEMFPRTPGIWLLHCHVTDHI HAGMETTYTVLQNEDTKSG (SEQ ID NO:416) corresponding to amino acids 208-994 of HSCP2_P16 (SEQ ID NO:316), wherein said first amino acid sequence and second amino acid sequence are contiguous and in a sequential order.

According to one embodiment, the present invention provides an isolated polypeptide comprising an edge portion of HSCP2_P16 (SEQ ID NO:316), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMH WTFNVECLTTDHYTGGMKQKYTVNQCRRQSEDSTFYLGERTYYIAAVEVEWDYSPQRE WEKELHHLQEQNVSNAFLDKGEFYIGSKYKKVVYRQYTDSTFRVPVERKAEEEHLGILG PQLHADVGDKVKIIFKNMATRPYSIHAHGVQTESSTVTPTLPGETLTYVWKIPERSGAGT EDSACIPWAYYSTVDQVKDLYSGLIGPLIVCRRPYLKVFNPRRKLEFALLFLVFDENESW YLDDNIKTYSDHPEKVNKDDEEFIESNKMHAINGRMFGNLQGLTMHVGDEVNWYLMG MGNEIDLHTVHFHGHSFQYKHRGVYSSDVFDIFPGTYQTLEMFPRTPGIWLLHCHVTDHI HAGMETTYTVLQNEDTKSG (SEQ ID NO:416) of HSCP2_P16 (SEQ ID NO:316).

According to one embodiment, the isolated polypeptide is a chimeric polypeptide comprising a first amino acid sequence being at least about 90% homologous to amino acids 1-131 of CERU_HUMAN (SEQ ID NO:308), which also corresponds to amino acids 1-131 of HSCP2_P18 (SEQ ID NO:317), a second bridging amino acid sequence comprising of A, and a third amino acid sequence being at least about 90% homologous to amino acids 262-1065 of CERU_HUMAN (SEQ ID NO:308), which also corresponds to amino acids 133-936 of HSCP2_P18 (SEQ ID NO:317), wherein said first amino acid sequence, second amino acid sequence and third amino acid sequence are contiguous and in a sequential order.

According to one embodiment, the present invention provides an isolated polypeptide comprising an edge portion of HSCP2_P18 (SEQ ID NO:317), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about 3 amino acids comprise EAV having a structure as follows (numbering according to HSCP2_P18 (SEQ ID NO:317): a sequence starting from any of amino acid numbers 131-x to 131; and ending at any of amino acid numbers 133+((n−3)−x), in which x varies from 0 to n−3.

According to one embodiment, the isolated polypeptide is a chimeric polypeptide comprising a a first amino acid sequence being at least about 90% homologous to amino acids 1-131 of Q6NSB2_HUMAN (SEQ ID NO:309), which also corresponds to amino acids 1-131 of HSCP2_P18 (SEQ ID NO:317), and a second amino acid sequence being at least about 70%, optionally at least about 80%, preferably at least about 85%, more preferably at least about 90% and most preferably at least about 95% homologous to a polypeptide having the sequence AVNGYTFGSLPGLSMCAEDRVKWYLFGMGNEVDVHAAFFHGQALTNKNYRIDTINLFP ATLFDAYMVAQNPGEWMLSCQNLNHLKAGLQAFFQVQECNKSSSKDNIRGKHVRHYYI AAEEIIWNYAPSGIDIFTKENLTAPGSDSAVFFEQGTTRIGGSYKKLVYREYTDASFTNRK ERGPEEEHLGILGPVIWAEVGDTIRVTFHNKGAYPLSIEPIGVRFNKNNEGTYYSPNYNPQ SRSVPPSASHVAPTETFTYEWTVPKEVGPTNADPVCLAKMYYSAVDPTKDIFTGLIGPMK ICKKGSLHANGRQKDVDKEFYLFPTVFDENESLLLEDNIRMFTTAPDQVDKEDEDFQESN KMHSMNGFMYGNQPGLTMCKGDSVVWYLFSAGNEADVHGIYFSGNTYLWRGERRDT ANLFPQTSLTLHMWPDTEGTFNVECLTTDHYTGGMKQKYTVNQCRRQSEDSTFYLGER TYYIAAVEVEWDYSPQREWEKELHHLQEQNVSNAFLDKGEFYIGSKYKKVVYRQYTDS TFRVPVEKEEEHLGILGPQLHADVGDKVKIIFKNMATRPYSIHAHGVQTESSTVTPTLP GETLTYVWKIPERSGAGTEDSACIPWAYYSTVDQVKDLYSGLIGPLIVCRRPYLKVFNPR RKLEFALLFLVFDENESWYLDDNIKTYSDHPEKVNKDDEEFIESNKMHAINGRMFGNLQ GLTMIIVGDEVNWYLMGMGNEIDLHTVHFHGHSFQYKHRGVYSSDVFDIFPGTYQTLE MFPRTPGIWLLHCHVTDHIHAGMETTYTVLQNEDTKSG (SEQ ID NO:417) corresponding to amino acids 132-936 of HSCP2_P18 (SEQ ID NO:317), wherein said first amino acid sequence and second amino acid sequence are contiguous and in a sequential order.

According to one embodiment, the present invention provides an isolated polypeptide comprising an edge portion of HSCP2_P18 (SEQ ID NO:317), comprising an amino acid sequence being at least about 70%, optionally at least about 80%, preferably at least about 85%, more preferably at least about 90% and most preferably at least about 95% homologous to the sequence AVNGYTFGSLPGLSMCAEDRVKWYLFGMGNEVDVHAAFFHGQALTNKNYRIDTINLFP ATLFDAYMVAQNPGEWMLSCQNLNHLKAGLQAFFQVQECNKSSSKDNIRGKHVRHYYI AAEEIIWNYAPSGIDIFTKENLTAPGSDSAVFFEQGTTRIGGSYKKLVYREYTDASFTNRK ERGPEEEHLGILGPVIWAEVGDTIRVTFHNKGAYPLSIEPIGVRFNKNEGTYYSPNYNPQ SRSVPPSASHVAPTETFTYEWTVPKEVGPTNADPVCLAKMYYSAVDPTKDIFTGLIGPMK ICKKGSLHANGRQKDVDKEFYLFPTVFDENESLLLEDNIRMFTTAPDQVDKEDEDFQESN KMHSMNGFMYGNQPGLTMCKGDSVVWYLFSAGNEADVHGIYFSGNTYLWRGERRDT ANLFPQTSLTLHMWPDTEGTFNVECLTTDHYTGGMKQKYTVNQCRRQSEDSTFYLGER TYYIAAVEVEWDYSPQREWEKELHHLQEQNVSNAFLDKGEFYIGSKYKKVVYRQYTDS TFRVPVERKAEEEIHLGILGPQLHADVGDKVKIIFKNMATRPYSIHAHGVQTESSTVTPTLP GETLTYVWIPERSGAGTEDSACIPWAYYSTVDQVKDLYSGLIGPLIVCRRPYLKVFNPR RKLEFALLFLVFDENESWYLDDNIKTYSDHPEKVNKDDEEFIESNKMHAINGRMFGNLQ GLTMHVGDEVNWYLMGMGNEIDLHTVHFHGHSFQYKHRGVYSSDVFDIFPGTYQTLE MFPRTPGIWLLHCHVTDHIHAGMETTYTVLQNEDTKSG (SEQ ID NO:417) of HSCP2_P18 (SEQ ID NO:317).

According to one embodiment, the present invention provides an isolated polypeptide comprising an edge portion of HSCP2_P21 (SEQ ID NO:318), comprising an amino acid sequence being at least about 70%, optionally at least about 80%, preferably at least about 85%, more preferably at least about 90% and most preferably at least about 95% homologous to the sequence THGGGGGGGAF (SEQ ID NO:418) of HSCP2_P21 (SEQ ID NO:318).

According to one embodiment, the present invention provides an isolated polypeptide comprising an edge portion of HSCP2_P21 (SEQ ID NO:318), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGTHG GGGGGGAF (SEQ ID NO:419) of HSCP2_P21 (SEQ ID NO:318).

According to one embodiment, the isolated polypeptide is a chimeric polypeptide comprising a first amino acid sequence being at least about 90% homologous to amino acids 1-852 of CERU_HUMAN (SEQ ID NO:308), which also corresponds to amino acids 1-852 of HSCP2_P21 (SEQ ID NO:318), and a second amino acid sequence being at least about 70%, optionally at least about 80%, preferably at least about 85%, more preferably at least about 90% and most preferably at least about 95% homologous to a polypeptide having the sequence THGGGGGGGAF (SEQ ID NO:418) corresponding to amino acids 853-863 of HSCP2_P21 (SEQ ID NO:318), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P21 (SEQ ID NO:318), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGTHG GGGGGGAF (SEQ ID NO:419) corresponding to amino acids 208-863 of HSCP2_P21 (SEQ ID NO:318), wherein said first amino acid sequence and second amino acid sequence are contiguous and in a sequential order.

According to another embodiment, the isolated polypeptide comprises an amino acid sequence as set forth in SEQ. ID NO: 318 (HSCP2_P21).

According to one embodiment, the present invention provides an isolated polypeptide comprising an edge portion of HSCP2_P23 (SEQ ID NO:319), comprising an amino acid sequence being at least about 70%, optionally at least about 80%, preferably at least about 85%, more preferably at least about 90% and most preferably at least about 95% homologous to the sequence CKYCIIHQSTKLF (SEQ ID NO:420) of HSCP2_P23 (SEQ ID NO:319).

According to one embodiment, the present invention provides an isolated polypeptide comprising an edge portion of HSCP2_P23 (SEQ ID NO:319), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHC KYCIIHQSTKLF (SEQ ID NO:421) of HSCP2_P23 (SEQ ID NO:319).

According to one embodiment, the isolated polypeptide is a chimeric polypeptide comprising a a first amino acid sequence being at least about 90% homologous to amino acids 1-621 of CERU_HUMAN (SEQ ID NO:308), which also corresponds to amino acids 1-621 of HSCP2_P23 (SEQ ID NO:319), and a second amino acid sequence being at least about 70%, optionally at least about 80%, preferably at least about 85%, more preferably at least about 90% and most preferably at least about 95% homologous to a polypeptide having the sequence CKYCIIHQSTKLF (SEQ ID NO:420) corresponding to amino acids 622-634 of HSCP2_P23 (SEQ ID NO:319), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P23 (SEQ ID NO:319), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHC KYCIIHQSTKLF (SEQ ID NO:421) corresponding to amino acids 208-634 of HSCP2_P23 (SEQ ID NO:319), wherein said first amino acid sequence and second amino acid sequence are contiguous and in a sequential order.

According to one embodiment, the present invention provides an isolated polypeptide comprising an edge portion of HSCP2_P24 (SEQ ID NO:320), comprising an amino acid sequence being at least about 70%, optionally at least about 80%, preferably at least about 85%, more preferably at least about 90% and most preferably at least about 95% homologous to the sequence EYTALCNK (SEQ ID NO:422) of HSCP2_P24 (SEQ ID NO:320).

According to one embodiment, the present invention provides an isolated polypeptide comprising an edge portion of HSCP2_P24 (SEQ ID NO:320), comprising an amino acid sequence being at least about 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSEY TALCNK (SEQ ID NO:423) of HSCP2_P24 (SEQ ID NO:320).

According to one embodiment, the isolated polypeptide is a chimeric polypeptide comprising a first amino acid sequence being at least about 90% homologous to amino acids 1-501 of CERU_HUMAN (SEQ ID NO:308), which also corresponds to amino acids 1-501 of HSCP2_P24 (SEQ ID NO:320), and a second amino acid sequence being at least about 70%, optionally at least about 80%, preferably at least about 85%, more preferably at least about 90% and most preferably at least about 95% homologous to a polypeptide having the sequence EYTALCNK (SEQ ID NO:422) corresponding to amino acids 502-509 of HSCP2_P24 (SEQ ID NO:320), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-207 of Q6NSB2_HUMAN (SEQ ID NO:309), which also corresponds to amino acids 1-207 of HSCP2_P24 (SEQ ID NO:320), and a second amino acid sequence being at least about 70%, optionally at least about 80%, preferably at least about 85%, more preferably at least about 90% and most preferably at least about 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSEY TALCNK (SEQ ID NO:423) corresponding to amino acids 208-509 of HSCP2_P24 (SEQ ID NO:320), wherein said first amino acid sequence and second amino acid sequence are contiguous and in a sequential order.

According to another embodiment, the isolated polypeptide comprises an amino acid sequence as set forth in SEQ. ID NO: 320 (HSCP2_P24).

According to one embodiment, the present invention provides an isolated polypeptide comprising an edge portion of HSCP2_P37 (SEQ ID NO:321), comprising an amino acid sequence being at least about 70%, optionally at least about 80%, preferably at least about 85%, more preferably at least about 90% and most preferably at least about 95% homologous to the sequence GHLP (SEQ ID NO:424) of HSCP2_P37 (SEQ ID NO:321).

According to one embodiment, the isolated polypeptide is a chimeric polypeptide comprising a first amino acid sequence being at least about 90% homologous to amino acids 1-49 of CERU_HUMAN (SEQ ID NO:308), which also corresponds to amino acids 1-49 of HSCP2_P37 (SEQ ID NO:321), and a second amino acid sequence being at least about 70%, optionally at least about 80%, preferably at least about 85%, more preferably at least about 90% and most preferably at least about 95% homologous to a polypeptide having the sequence GHLP (SEQ ID NO:424) corresponding to amino acids 50-53 of HSCP2_P37 (SEQ ID NO:321), wherein said first amino acid sequence and second amino acid sequence are contiguous and in a sequential order.

According to another embodiment, the isolated polypeptide comprises an amino acid sequence as set forth in SEQ. ID NO: 321 (HSCP2_P37).

According to one embodiment, the present invention provides an isolated polypeptide comprising an edge portion of HSCP2_P39 (SEQ ID NO:322), comprising an amino acid sequence being at least about 70%, optionally at least about 80%, preferably at least about 85%, more preferably at least about 90% and most preferably at least about 95% homologous to the sequence CEWIHFWKSPRTLHVC (SEQ ID NO:425) of HSCP2_P39 (SEQ ID NO:322).

According to one embodiment, the isolated polypeptide is a chimeric polypeptide comprising a first amino acid sequence being at least about 90% homologous to amino acids 1-49 of CERU_HUMAN (SEQ ID NO:308), which also corresponds to amino acids 1-49 of HSCP2_P39 (SEQ ID NO:322), and a second amino acid sequence being at least about 70%, optionally at least about 80%, preferably at least about 85%, more preferably at least about 90% and most preferably at least about 95% homologous to a polypeptide having the sequence CEWIHFWKSPRTLHVC (SEQ ID NO:425) corresponding to amino acids 50-65 of HSCP2_P39 (SEQ ID NO:322), wherein said first amino acid sequence and second amino acid sequence are contiguous and in a sequential order.

According to another embodiment, the isolated polypeptide comprises an amino acid sequence as set forth in SEQ. ID NO: 322 (HSCP2_P39).

According to certain embodiments, the present invention now discloses a novel cluster designated herein S56200, comprising novel amino acid and nucleic acid sequences that are variants of the known protein Myeloperoxidase precursor (SEQ ID NO:361) (SwissProt accession identifier PERM_HUMAN (SEQ ID NO:371); known also according to the synonyms EC 1.11.1.7; MPO). The novel variant polynucleotides and polypeptides described by the present invention are useful as diagnostic markers, preferably as serum markers.

According to the present invention S56200 variants are predicted to be overexpressed in cancerous tissues, particularly in cancerous lung tissues, Lymphoma and Leukemia, and thus can be used for the diagnosis, prognosis, treatment selection, and treatment monitoring and/or assessment of cancers, particularly lung cancer, Lymphoma and Leukemia.

According to the present invention S56200 variants are predicted to be overexpressed in cardiovascular and cerebrovascular conditions, and thus can be used for the diagnosis, prognosis, treatment selection, and treatment monitoring and/or assessment of cardiovascular and cerebrovascular conditions, particularly atherosclerotic cardiovascular disease, coronary artery disease, heart failure, myocardial infarction, cardiomyopathy, myocarditis, Congestive Heart Failure (CHF), acute and chronic inflammation and stroke.

According to one embodiment, the present invention provides an isolated polypeptide comprising an edge portion of S56200_P6 (SEQ ID NO:365), comprising an amino acid sequence being at least about 70%, optionally at least about 80%, preferably at least about 85%, more preferably at least about 90% and most preferably at least about 95% homologous to the sequence FGGRTRVCSACSSDRPWPRSHCPGSSATTQASPPCLRTTSSCPTHIPGTLSTAVHFLH (SEQ ID NO:426) of S56200_P6 (SEQ ID NO:365).

According to one embodiment, the present invention provides an isolated polypeptide comprising a head of S56200_P6 (SEQ ID NO:365), comprising a polypeptide being at least about 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) of S56200_P6 (SEQ ID NO:365).

According to one embodiment, the present invention provides an isolated polypeptide comprising an edge portion of S56200_P6 (SEQ ID NO:365), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise RR, having a structure as follows: a sequence starting from any of amino acid numbers 183-x to 183; and ending at any of amino acid numbers 184+((n−2)−x), in which x varies from 0 to n−2.

According to one embodiment, the isolated polypeptide is a chimeric polypeptide comprising a first amino acid sequence being at least about 90% homologous to amino acids 1-666 of PERM_HUMAN (SEQ ID NO:371), which also corresponds to amino acids 1-666 of S56200_P6 (SEQ ID NO:365), and a second amino acid sequence being at least about 70%, optionally at least about 80%, preferably at least about 85%, more preferably at least about 90% and most preferably at least about 95% homologous to a polypeptide having the sequence FGGRTRVCSACSSDRPWPRSHCPGSSATTQASPPCLRTTSSCPTHIPGTLSTAVHFLH (SEQ ID NO:426) corresponding to amino acids 667-724 of S56200_P6 (SEQ ID NO:365), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-183 of P05164-3 (SEQ ID NO:364), which also corresponds to amino acids 1-183 of S56200_P6 (SEQ ID NO:365), a second amino acid sequence being at least about 90% homologous to amino acids 216-698 of P05164-3 (SEQ ID NO:364), which also corresponds to amino acids 184-666 of S56200_P6 (SEQ ID NO:365), and a third amino acid sequence being at least about 70%, optionally at least about 80%, preferably at least about 85%, more preferably at least about 90% and most preferably at least about 95% homologous to a polypeptide having the sequence FGGRTRVCSACSSDRPWPRSHCPGSSATTQASPPCLRTTSSCPTHIPGTLSTAVHFLH (SEQ ID NO:426) corresponding to amino acids 667-724 of S56200_P6 (SEQ ID NO:365), wherein said first amino acid sequence, second amino acid sequence and third amino acid sequence are contiguous and in a sequential order.

According to one embodiment, the isolated polypeptide is a chimeric polypeptide comprising a first amino acid sequence being at least about 70%, optionally at least about 80%, preferably at least about 85%, more preferably at least about 90% and most preferably at least about 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) corresponding to amino acids 1-95 of S56200_P6 (SEQ ID NO:365), a second amino acid sequence being at least about 90% homologous to amino acids 1-571 of P05164-2 (SEQ ID NO:362), which also corresponds to amino acids 96-666 of S56200_P6 (SEQ ID NO:365), and a third amino acid sequence being at least about 70%, optionally at least about 80%, preferably at least about 85%, more preferably at least about 90% and most preferably at least about 95% homologous to a polypeptide having the sequence FGGRTRVCSACSSDRPWPRSHCPGSSATTQASPPCLRTTSSCPTHIPGTLSTAVHFLH (SEQ ID NO:426) corresponding to amino acids 667-724 of S56200_P6 (SEQ ID NO:365), wherein said first amino acid sequence, second amino acid sequence and third amino acid sequence are contiguous and in a sequential order.

According to another embodiment, the isolated polypeptide comprises an amino acid sequence as set forth in SEQ. ID NO: 365 (S56200_P6).

According to one embodiment, the present invention provides an isolated polypeptide comprising an edge portion of S56200_P7 (SEQ ID NO:366), comprising an amino acid sequence being at least about 70%, optionally at least about 80%, preferably at least about 85%, more preferably at least about 90% and most preferably at least about 95% homologous to the sequence KQDLGQERVG (SEQ ID NO:428) of S56200_P7 (SEQ ID NO:366).

According to one embodiment, the present invention provides an isolated polypeptide comprising an edge portion of S56200_P7 (SEQ ID NO:366), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise RR, having a structure as follows: a sequence starting from any of amino acid numbers 183-x to 183; and ending at any of amino acid numbers 184+((n−2)−x), in which x varies from 0 to n−2.

According to one embodiment, the present invention provides an isolated polypeptide comprising a head of S56200_P7 (SEQ ID NO:366), comprising a polypeptide being at least about 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:429) of S56200_P7 (SEQ ID NO:366).

According to one embodiment, the isolated polypeptide is a chimeric polypeptide comprising a first amino acid sequence being at least about 90% homologous to amino acids 1-541 of PERM_HUMAN (SEQ ID NO:371), which also corresponds to amino acids 1-541 of S56200_P7 (SEQ ID NO:366), and a second amino acid sequence being at least about 70%, optionally at least about 80%, preferably at least about 85%, more preferably at least about 90% and most preferably at least about 95% homologous to a polypeptide having the sequence KQDLGQERVG (SEQ ID NO:428) corresponding to amino acids 542-551 of S56200_P7 (SEQ ID NO:366), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-183 of P05164-3 (SEQ ID NO:364), which also corresponds to amino acids 1-183 of S56200_P7 (SEQ ID NO:366), a second amino acid sequence being at least about 90% homologous to amino acids 184-541 of S56200_P7 (SEQ ID NO:366), and a third amino acid sequence being at least about 70%, optionally at least about 80%, preferably at least about 85%, more preferably at least about 90% and most preferably at least about 95% homologous to a polypeptide having the sequence KQDLGQERVG (SEQ ID NO:428) corresponding to amino acids 542-551 of S56200_P7 (SEQ ID NO:366), wherein said first amino acid sequence, second amino acid sequence and third amino acid sequence are contiguous and in a sequential order.

According to one embodiment, the isolated polypeptide is a chimeric polypeptide comprising a first amino acid sequence being at least about 70%, optionally at least about 80%, preferably at least about 85%, more preferably at least about 90% and most preferably at least about 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:429) corresponding to amino acids 1-95 of S56200_P7 (SEQ ID NO:366), a second amino acid sequence being at least about 90% homologous to amino acids 1-446 of P05164-2 (SEQ ID NO:362), which also corresponds to amino acids 96-541 of S56200_P7 (SEQ ID NO:366), and a third amino acid sequence being at least about 70%, optionally at least about 80%, preferably at least about 85%, more preferably at least about 90% and most preferably at least about 95% homologous to a polypeptide having the sequence KQDLGQERVG (SEQ ID NO:428) corresponding to amino acids 542-551 of S56200_P7 (SEQ ID NO:366), wherein said first amino acid sequence, second amino acid sequence and third amino acid sequence are contiguous and in a sequential order.

According to another embodiment, the isolated polypeptide comprises an amino acid sequence as set forth in SEQ. ID NO: 365 (S56200_P6).

According to one embodiment, the present invention provides an isolated polypeptide comprising an edge portion of S56200_P10 (SEQ ID NO:367), comprising an amino acid sequence being at least about 70%, optionally at least about 80%, preferably at least about 85%, more preferably at least about 90% and most preferably at least about 95% homologous to the sequence DHHLPGLPAPGAGANGHEEVPAHVPFLQ (SEQ ID NO:430) of S56200_P10 (SEQ ID NO:367).

According to one embodiment, the present invention provides an isolated polypeptide comprising an edge portion of S56200_P10 (SEQ ID NO:367), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise RR, having a structure as follows: a sequence starting from any of amino acid numbers 183-x to 183; and ending at any of amino acid numbers 184+((n−2)−x), in which x varies from 0 to n−2.

According to one embodiment, the present invention provides an isolated polypeptide comprising a head of S56200_P10 (SEQ ID NO:367), comprising a polypeptide being at least about 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:431) of S56200_P10 (SEQ ID NO:367).

According to one embodiment, the isolated polypeptide is a chimeric polypeptide comprising a first amino acid sequence being at least about 90% homologous to amino acids 1-401 of PERM_HUMAN (SEQ ID NO:371), which also corresponds to amino acids 1-401 of S56200_P10 (SEQ ID NO:367), and a second amino acid sequence being at least about 70%, optionally at least about 80%, preferably at least about 85%, more preferably at least about 90% and most preferably at least about 95% homologous to a polypeptide having the sequence DHHLPGLPAPGAGANGHEEVPAHVPFLQ (SEQ ID NO:430) corresponding to amino acids 402-429 of S56200_P10 (SEQ ID NO:367), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-183 of P05164-3 (SEQ ID NO:364), which also corresponds to amino acids 1-183 of S56200_P10 (SEQ ID NO:367), a second amino acid sequence being at least about 90% homologous to amino acids 216-433 of P05164-3 (SEQ ID NO:364), which also corresponds to amino acids 184-401 of S56200_P10 (SEQ ID NO:367), and a third amino acid sequence being at least about 70%, optionally at least about 80%, preferably at least about 85%, more preferably at least about 90% and most preferably at least about 95% homologous to a polypeptide having the sequence DHHLPGLPAPGAGANGHEEVPAHVPFLQ (SEQ ID NO:430) corresponding to amino acids 402-429 of S56200_P10 (SEQ ID NO:367), wherein said first amino acid sequence, second amino acid sequence and third amino acid sequence are contiguous and in a sequential order.

According to one embodiment, the isolated polypeptide is a chimeric polypeptide comprising a first amino acid sequence being at least about 70%, optionally at least about 80%, preferably at least about 85%, more preferably at least about 90% and most preferably at least about 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:431) corresponding to amino acids 1-95 of S56200_P10 (SEQ ID NO:367), a second amino acid sequence being at least about 90% homologous to amino acids 1-306 of P05164-2 (SEQ ID NO:362), which also corresponds to amino acids 96-401 of S56200_P10 (SEQ ID NO:367), and a third amino acid sequence being at least about 70%, optionally at least about 80%, preferably at least about 85%, more preferably at least about 90% and most preferably at least about 95% homologous to a polypeptide having the sequence DHHLPGLPAPGAGANGHEEVPAHVPFLQ (SEQ ID NO:430) corresponding to amino acids 402-429 of S56200_P10 (SEQ ID NO:367), wherein said first amino acid sequence, second amino acid sequence and third amino acid sequence are contiguous and in a sequential order.

According to one embodiment, the present invention provides an isolated polypeptide comprising an edge portion of S56200_P21 (SEQ ID NO:368), comprising an amino acid sequence being at least about 70%, optionally at least about 80%, preferably at least about 85%, more preferably at least about 90% and most preferably at least about 95% homologous to the sequence QLWGGDQRWHRRCSLLEPQGHPFQ (SEQ ID NO:432) of S56200_P21 (SEQ ID NO:368).

According to one embodiment, the present invention provides an isolated polypeptide comprising an edge portion of S56200_P21 (SEQ ID NO:368), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise RR, having a structure as follows: a sequence starting from any of amino acid numbers 183-x to 183; and ending at any of amino acid numbers 184+((n−2)−x), in which x varies from 0 to n−2.

According to one embodiment, the present invention provides an isolated polypeptide comprising a head of S56200_P21 (SEQ ID NO:368), comprising a polypeptide being at least about 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:433) of S56200_P21 (SEQ ID NO:368).

According to one embodiment, the isolated polypeptide is a chimeric polypeptide comprising a first amino acid sequence being at least about 90% homologous to amino acids 1-402 of PERM_HUMAN (SEQ ID NO:371), which also corresponds to amino acids 1-402 of S56200_P21 (SEQ ID NO:368), and a second amino acid sequence being at least about 70%, optionally at least about 80%, preferably at least about 85%, more preferably at least about 90% and most preferably at least about 95% homologous to a polypeptide having the sequence QLWGGDQRWHRRCSLLEPQGHPFQ (SEQ ID NO:432) corresponding to amino acids 403-426 of S56200_P21 (SEQ ID NO:368), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-183 of P05164-3 (SEQ ID NO:364), which also corresponds to amino acids 1-183 of S56200_P21 (SEQ ID NO:368), a second amino acid sequence being at least about 90% homologous to amino acids 216-434 of P05164-3 (SEQ ID NO:364), which also corresponds to amino acids 184-402 of S56200_P21 (SEQ ID NO:368), and a third amino acid sequence being at least about 70%, optionally at least about 80%, preferably at least about 85%, more preferably at least about 90% and most preferably at least about 95% homologous to a polypeptide having the sequence QLWGGDQRWHRRCSLLEPQGHPFQ (SEQ ID NO:432) corresponding to amino acids 403-426 of S56200_P21 (SEQ ID NO:368), wherein said first amino acid sequence, second amino acid sequence and third amino acid sequence are contiguous and in a sequential order.

According to one embodiment, the isolated polypeptide is a chimeric polypeptide comprising a first amino acid sequence being at least about 70%, optionally at least about 80%, preferably at least about 85%, more preferably at least about 90% and most preferably at least about 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:433) corresponding to amino acids 1-95 of S56200_P21 (SEQ ID NO:368), a second amino acid sequence being at least about 90% homologous to amino acids 1-307 of P05164-2 (SEQ ID NO:362), which also corresponds to amino acids 96-402 of S56200_P21 (SEQ ID NO:368), and a third amino acid sequence being at least about 70%, optionally at least about 80%, preferably at least about 85%, more preferably at least about 90% and most preferably at least about 95% homologous to a polypeptide having the sequence QLWGGDQRWHRRCSLLEPQGHPFQ (SEQ ID NO:432) corresponding to amino acids 403-426 of S56200_P21 (SEQ ID NO:368), wherein said first amino acid sequence, second amino acid sequence and third amino acid sequence are contiguous and in a sequential order.

According to another embodiment, the isolated polypeptide comprises an amino acid sequence as set forth in SEQ. ID NO: 368 (S56200_P21).

According to one embodiment, the present invention provides an isolated polypeptide comprising edge portion of S56200_P24 (SEQ ID NO:369), comprising an amino acid sequence being at least about 70%, optionally at least about 80%, preferably at least about 85%, more preferably at least about 90% and most preferably at least about 95% homologous to the sequence VALLPAHCLGWERGDCFWKGPSPFCAQVSFPRR (SEQ ID NO:434) of S56200_P24 (SEQ ID NO:369).

According to one embodiment, the present invention provides an isolated polypeptide comprising an edge portion of S56200_P24 (SEQ ID NO:369), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least about two amino acids comprise RR, having a structure as follows: a sequence starting from any of amino acid numbers 183-x to 183; and ending at any of amino acid numbers 184+((n−2)−x), in which x varies from 0 to n−2.

According to one embodiment, the present invention provides an isolated polypeptide comprising a head of S56200_P24 (SEQ ID NO:369), comprising a polypeptide being at least about 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:435) of S56200_P24 (SEQ ID NO:369).

According to one embodiment, the present invention provides an isolated polypeptide comprising an amino acid sequence being at least about 70%, optionally at least about 80%, preferably at least about 85%, more preferably at least about 90% and most preferably at least about 95% homologous to the sequence RWGTRHCPALGKDVPGLSEKQQAAGSLKHQAAASQRLSQPHGTPILLQAAGGSHQDGG EGR (SEQ ID NO:437) of S56200_P31 (SEQ ID NO:370).

According to one embodiment, the isolated polypeptide is a chimeric polypeptide comprising a first amino acid sequence being at least about 90% homologous to amino acids 1-295 of PERM_HUMAN (SEQ ID NO:371), which also corresponds to amino acids 1-295 of S56200_P24 (SEQ ID NO:369), and a second amino acid sequence being at least about 70%, optionally at least about 80%, preferably at least about 85%, more preferably at least about 90% and most preferably at least about 95% homologous to a polypeptide having the sequence VALLPAHCLGWERGDCFWKGPSPFCAQVSFPRR (SEQ ID NO:434) corresponding to amino acids 296-328 of S56200_P24 (SEQ ID NO:369), wherein said first amino acid sequence and second amino acid sequence are contiguous and in a sequential order.

According to one embodiment, the isolated polypeptide is a chimeric polypeptide comprising a first amino acid sequence being at least about 90% homologous to amino acids 1-183 of P05164-3 (SEQ ID NO:364), which also corresponds to amino acids 1-183 of S56200_P24 (SEQ ID NO:369), a second amino acid sequence being at least about 90% homologous to amino acids 216-327 of P05164-3 (SEQ ID NO:364), which also corresponds to amino acids 184-295 of S56200_P24 (SEQ ID NO:369), and a third amino acid sequence being at least about 70%, optionally at least about 80%, preferably at least about 85%, more preferably at least about 90% and most preferably at least about 95% homologous to a polypeptide having the sequence VALLPAHCLGWERGDCFWKGPSPFCAQVSFPRR (SEQ ID NO:434) corresponding to amino acids 296-328 of S56200_P24 (SEQ ID NO:369), wherein said first amino acid sequence, second amino acid sequence and third amino acid sequence are contiguous and in a sequential order.

According to one embodiment, the isolated polypeptide is a chimeric polypeptide comprising a first amino acid sequence being at least about 70%, optionally at least about 80%, preferably at least about 85%, more preferably at least about 90% and most preferably at least about 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:435) corresponding to amino acids 1-95 of S56200_P24 (SEQ ID NO:369), a second amino acid sequence being at least about 90% homologous to amino acids 1-200 of P05164-2 (SEQ ID NO:362), which also corresponds to amino acids 96-295 of S56200_P24 (SEQ ID NO:369), and a third amino acid sequence being at least about 70%, optionally at least about 80%, preferably at least about 85%, more preferably at least about 90% and most preferably at least about 95% homologous to a polypeptide having the sequence VALLPAHCLGWERGDCFWKGPSPFCAQVSFPRR (SEQ ID NO:434) corresponding to amino acids 296-328 of S56200_P24 (SEQ ID NO:369), wherein said first amino acid sequence, second amino acid sequence and third amino acid sequence are contiguous and in a sequential order.

According to one embodiment, the isolated polypeptide is a chimeric polypeptide comprising a first amino acid sequence being at least about 90% homologous to amino acids 1-82 of PERM_HUMAN (SEQ ID NO:371), which also corresponds to amino acids 1-82 of S56200_P31 (SEQ ID NO:370), and a second amino acid sequence being at least about 70%, optionally at least about 80%, preferably at least about 85%, more preferably at least about 90% and most preferably at least about 95% homologous to a polypeptide having the sequence RWGTRHCPALGKDVPGLSEKQQAAGSLKHQAAASQRLSQPHGTPILLQAAGGSHQDGG EGR (SEQ ID NO:437) corresponding to amino acids 83-143 of S56200_P31 (SEQ ID NO:370), wherein said first amino acid sequence and second amino acid sequence are contiguous and in a sequential order.

According to another embodiment, the isolated polypeptide comprises an amino acid sequence as set forth in SEQ. ID NO: 370 (S56200_P31).

According to certain embodiments, the polypeptides of this invention comprise variants of known proteins, and in other embodiments the polypeptides of this invention comprise splice variants of native proteins expressed in a given subject. In some embodiments, the polypeptides may be obtained through known protein evolution techniques available in the art. In other embodiments, the polypeptides of this invention may be obtained via rational design, based on a particular native polypeptide sequence.

According to another aspect the present invention provides antibodies or antibody fragments specifically interacting with or recognizing a polypeptide of this invention.

According to certain embodiments, the antibody recognizes one or more epitopes (antigen determinants) contained within the polypeptides of this invention, wherein such that binding of the antibody to an epitope distinguish between the splice variants of the present invention and a known polypeptide or protein. Reference to the antibody property of “specific interaction” or “recognition” is to be understood as including covalent and non-covalent associations with a variance of affinity over several orders of magnitude. These terms are to be understood as relative with respect to an index molecule, for which the antibody is thought to have little to no specific interaction or recognition. In one embodiment, the antibodies specifically interact or recognize a particular antigen determinant.

In certain embodiments, the antibodies or antibody fragments of this invention recognize or interact with a polypeptide or protein of the invention, while not substantially recognize or interact with other molecules, even when present in the same sample, for example a biological sample. According to one embodiment, the antibodies of this invention have a specificity such that the specific interaction with or binding to the antigen is at least about 2, or in another embodiment, at least about 5, or in still further embodiment, at least about 10-fold greater than interaction or binding observed under the same reaction conditions with a molecule that does not include the antigenic determinant.

According to certain embodiments, the antibodies are useful in detecting qualitative and/or quantitative changes in the expression of the polypeptides or polynucleotides of this invention. In some embodiments, changes in expression are associated with a particular disease or disorder, such that detection of the changes comprises a diagnostic method of the present invention.

According to other embodiments, the present invention provides an antibody capable of specifically binding to at least one epitope of a polypeptide comprising an amino acid sequence as set forth in any one of SEQ. ID NOs: 91-96, 144-155, 180-183, 246-249, 310-322, 365-370.

According to additional aspect the present invention provides a diagnostic kit for detecting a disease, comprising markers and reagents for detecting qualitative and/or quantitative changes in the expression of a polypeptide or a polynucleotide of this invention.

According to on embodiment, the kit comprises markers and reagents for detecting the changes by employing a NAT-based technology. In one embodiment, the NAT-based assay is selected from the group consisting of a PCR, Real-Time PCR, LCR, Self-Sustained Synthetic Reaction, Q-Beta Replicase, Cycling Probe Reaction, Branched DNA, RFLP analysis, DGGE/TGGE, Single-Strand Conformation Polymorphism, Dideoxy Fingerprinting, Microarrays, Fluorescence In Situ Hybridization or Comparative Genomic Hybridization.

According to certain currently preferred embodiments, the kit comprises at least one nucleotide probe or primer. In one embodiment, the kit comprises at least one primer pair capable of selectively hybridizing to a nucleic acid sequence according to the teaching of the present invention. In another embodiment, the kit comprises at least one oligonucleotide capable of selectively hybridizing to a nucleic acid sequence according to the teaching of the present invention.

According to other currently preferred embodiments, the kit comprises an antibody capable of recognizing or interacting with a polypeptide or protein of the present invention. According to certain embodiments, the kit further comprises at least one reagent for performing an ELISA, an RIA, a slot blot, an immunohistochemical assay, FACS, in-vivo imaging, a radio-imaging assay, or a Western blot.

The present invention further provides diagnostic methods for screening for a disease, disorder or conditions, comprising the detection of a polypeptide or polynucleotide of this invention, whereby expression, or relative changes in expression of the polypeptide or polynucleotide herald the onset, severity, or prognosis of an individual with regard to a particular disease, disorder or condition. The detection may comprise detection of the expression of a specific splice variant, or other polypeptide or polynucleotide of this invention, via any means known in the art, and as described herein.

As used herein, the term “screening for a disease” encompasses diagnosing the presence of a disease, its prognosis and/or severity, as well as selecting a therapy or a treatment and monitoring the treatment of the disease. According to certain currently preferred embodiments, the disease is a marker-detectable disease, wherein the marker is a polynucleotide, polypeptide or protein according to the present invention.

Thus, according to certain aspects, the present invention provides methods for screening for a marker detectable disease, comprising detecting in a subject or in a sample obtained from the subject at least one transcript and/or protein or polypeptide being a member of a cluster selected from the group consisting of cluster AA340453, cluster AA703666, cluster AI590292, cluster HUMUMOD, cluster HSCP2, cluster S56200, or any combination thereof. According to certain currently preferred embodiments, the method comprises detecting the expression of a splice variant transcript or a product thereof.

According to one aspect, the present invention provide a method for screening for a marker detectable disease in a subject, comprising (a) obtaining a sample from the subject and (b) detecting in the sample at least one polynucleotide and/or polypeptide being a member of a cluster selected from the group consisting of cluster AA340453, cluster AA703666, cluster AI590292, cluster HUMUMOD, cluster HSCP2, cluster S56200, or any combination thereof. According to one embodiment, the presence of the polynucleotide or polypeptide in the sample is indicative of the presence of the disease and/or its severity and/or its progress. According to another embodiment, a change in the level of the polynucleotide or polypeptide in the sample compared to its level in a sample obtained from a healthy subject is indicative of the presence of the disease and/or its severity. According to another embodiment, a change in the level of the polynucleotide or polypeptide in the sample compared to its level in a sample previously obtained from said subject is indicative of the presence of the disease, its severity and/or the progress of the disease.

According to one embodiment, the present invention provides a method for screening for a cardiovascular disease in a subject, comprising (a) obtaining a sample from the subject and (b) detecting in the sample at least one polypeptide being a member of cluster S56200. According to one embodiment, the presence of the polypeptide in the sample is indicative of the presence of the disease and/or its severity and/or its progress. According to another embodiment, a change in the level of the polypeptide in the sample compared to its level in a sample obtained from a healthy subject is indicative of the presence of the disease and/or its severity. According to another embodiment, a change in the level of the polypeptide in the sample compared to its level in a sample previously obtained from said subject is indicative of the presence of the disease, its severity and/or the progress of the disease. According to currently preferred embodiments, the sample is a serum sample.

According to other embodiments, the cardiovascular disease include inter alia, myocardial infarct, acute coronary syndrome, coronary artery disease, angina pectoris (stable and unstable), cardiomyopathy, myocarditis, congestive heart failure or any type of heart failure and reinfarction. According to other embodiments, the method is useful for the assessment of thrombolytic therapy, assessment of myocardial infarct size, differential diagnosis between heart-related versus lung-related conditions (such as pulmonary embolism), the differential diagnosis of Dyspnea, cardiac valves related conditions, vascular disease, or any combination thereof. In further embodiments, the polypeptides of the cluster S56200, or a combination thereof, are useful in the diagnosis, treatment or assessment of the prognosis of a subject with congestive heart failure (CHF). According to still other embodiments, they are useful in the diagnosis, treatment or assessment of the prognosis of a subject with sudden cardiac death, from arrhythmia or any other heart related reason; rejection of a transplanted heart; conditions that lead to heart failure including but not limited to myocardial infarction, angina, arrhythmias, valvular diseases, atrial and/or ventricular septal defects; conditions that cause atrial and or ventricular wall volume overload, including but not limited to systemic arterial hypertension, pulmonary hypertension and pulmonary embolism; conditions which have similar clinical symptoms as heart failure and as states that cause atrial and or ventricular pressure-overload, where the differential diagnosis between these conditions to the latter is of clinical importance including but not limited to breathing difficulty and/or hypoxia due to pulmonary disease, anemia or anxiety.

These markers are specifically released to the bloodstream under conditions of cardiac disease and/or cardiac pathology, including but not limited to cardiac damage, and/or are otherwise expressed at a much higher level and/or specifically expressed in heart. The method of the present invention identifies clusters (genes) which are characterized in that the transcripts are differentially expressed in heart muscle tissue compared with other normal tissues, preferably in comparison to skeletal muscle tissue. In acute conditions under which heart muscle tissue experiences hypoxia (with or without necrosis), intracellular proteins that are not normally secreted can leak through the cell membrane to the extracellular space. Therefore, heart muscle tissue differentially expressed proteins, as through analysis of EST expression, are potential acute heart damage markers.

Each polypeptide of the S56200 variants described herein as a marker for cardiovascular conditions, can be used alone or in combination with one or more other variant markers described herein, and/or in combination with known markers for cardiovascular conditions, including but not limited to Heart-type fatty acid binding protein (H-FABP), Angiotensin, C-reactive protein (CRP), myeloperoxidase (MPO), and/or in combination with the known protein(s) for the variant marker as described herein.

In some embodiments, detecting in the sample at least one polypeptide being a member of a cluster selected from the group consisting of cluster S56200, or any combination thereof, or relative changes in expression of the genes, their products or certain variants thereof herald the onset, severity or prognosis of cerebrovascular disease in a subject. In some embodiments, the polypeptides, polynucleotides and/or methods of this invention may be useful in the treatment, diagnosis or prognosis assessment of any cerebrovascular disease, including, inter alia, stroke, including any type of stroke or neural tissue injury, or any type of cerebrovascular accident, ischemic stroke, hemorrhagic stroke or transient ischemic attacks, thrombotic, embolic, lacunar or hypoperfusion types of strokes, brain trauma, etc. In some embodiments, the polypeptides, polynucleotides and/or methods of this invention may be useful in the establishment of the timing of stroke; the type of stroke; the extent of tissue damage as a result of the stroke; response to immediate treatments that are meant to alleviate the extent of stroke and brain damage, when available, or any combination thereof.

In some embodiments, the polypeptides, polynucleotides and/or methods of this invention may be useful in the diagnosis of stroke and indication if an ischemic stroke has occurred; or a hemorrhagic stroke has occurred; or prognosis of a subsequent cerebral vasospasm; etc.

In some embodiments, the polypeptides, polynucleotides and/or methods of this invention may be useful in identifying a patient at risk for cerebral vasospasm. Such methods preferably comprise comparing an amount of one or more marker(s) predictive of a subsequent cerebral vasospasm in a test sample from a patient diagnosed with a subarachnoid hemorrhage. Such markers may be one or more markers related to blood pressure regulation, markers related to inflammation, markers related to apoptosis, and/or specific markers of neural tissue injury.

In some embodiments, the polypeptides, polynucleotides and/or methods of this invention may be useful in the diagnosis, treatment or assessment of the prognosis of a subject with acute and chronic inflammation, and/or CVS diseases, and in some embodiments, the marker comprises one or more of S56200 variants, including for a spectrum of diseases where an inflammatory process plays a substantial role. In some embodiments, the polypeptides, polynucleotides and/or methods of this invention may be useful in the diagnosis, treatment or assessment of the prognosis of a subject with hypercholesterolemia, diabetes, atherosclerosis, inflammation that involves blood vessels—whether acute or chronic including but not limited to the coronary arteries and blood vessels of the brain, myocardial infarction, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including but not limited to coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina; rheumatic/autoimmune diseases that involve systemic immune reaction including but not limited to rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus; acute and/or chronic infective processes that involve systemic immune reaction including but not limited to pneumonia, bacteremia, sepsis, pyelonephritis, cellulitis, osteomyelitis, meningitis and viral hepatitis; malignant and idiopathic processes that involve systemic immune reaction and/or proliferation of immune cells including but not limited to granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, amyloidosis, lymphoma, histiocytosis, renal cell carcinoma and paraneoplastic syndromes; conditions where CRP was shown to have a positive correlation with the presence of the condition including but not limited to weight loss, anorexia-cachexia syndrome, extent of disease, recurrence in advanced cancer, diabetes (types 1 & 2), obesity, hypertension, preterm delivery; conditions which have similar symptoms, signs and complications as the conditions above and where the differential diagnosis between them and the conditions above is of clinical importance including but not limited to: other (non vascular) causes of heart disease, renal disease and cerebral disease; other (non rheumatic) causes of arthropathy and musculoskeletal pain; other causes of non-specific symptoms and signs such as fever of unknown origin, loss of appetite, weight loss, nonspecific pains, breathing difficulties, anxiety, or any combination thereof, or any disease disorder or condition associated with inflammation.

The present invention further discloses that detecting in a subject at least one polypeptide or polynucleotide of cluster S56200, are indicative of cancer, particularly lung cancer. Detecting the presence of the polynucleotide or polypeptide in the subject or detecting a relative change in their expression and/or level compared to a healthy subject or compared to their expression and/or level in said subject at an earlier stage is indicative of the presence, onset, severity or prognosis, and/or staging, and/or progression, of lung cancer in said subject. These polynucleotides and polypeptides of cluster S56200 are also useful for treatment selection and treatment monitoring of lung cancer, which may be an invasive lung cancer and/or metastatic lung cancer.

Thus, according to another aspect, the present invention provides a method for screening for a lung cancer in a subject, comprising detecting in the subject at least one polynucleotide and/or polypeptide being a member of cluster S56200.

In other aspects, the present invention discloses that detection of polypeptides or polynucleotides of cluster HSCP2 variants, or relative changes in expression and/or level of these variants and their products is indicative of the presence, onset, severity or prognosis, and/or staging, and/or progression of cancer, including but not limited to ovarian cancer, or lung cancer in a subject. In some embodiments, the polypeptides, polynucleotides and/or methods of this invention may be useful in the treatment selection and monitoring, diagnosis or prognosis assessment of cancer, including but not limited to ovarian cancer or lung cancer, or ovarian or lung cancer invasion and metastasis.

With regard to lung cancer, the disease is selected from the group consisting of invasive or metastatic lung cancer; squamous cell lung carcinoma, lung adenocarcinoma, carcinoid, small cell lung cancer or non-small cell lung cancer; detection of overexpression in lung metastasis (vs. primary tumor); detection of overexpression in lung cancer, for example non small cell lung cancer, for example adenocarcinoma, squamous cell cancer or carcinoid, or large cell carcinoma; identification of a metastasis of unknown origin which originated from a primary lung cancer; assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors; distinguishing between different types of lung cancer, therefore potentially affecting treatment choice (e.g. small cell vs. non small cell tumors); analysis of unexplained dyspnea and/or chronic cough and/or hemoptysis; differential diagnosis of the origin of a pleural effusion; diagnosis of conditions which have similar symptoms, signs and complications as lung cancer and where the differential diagnosis between them and lung cancer is of clinical importance including but not limited to: non-malignant causes of lung symptoms and signs, including but not limited to: lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or detecting a cause of any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

The polypeptides and/or polynucleotides of cluster HSCP2 and/or S56200 used as markers for lung cancer can be used alone or in combination with one or more alternative polynucleotides or polypeptides described herein, and/or in combination with known markers for lung cancer, including but not limited to CEA, CA15-3, Beta-2-microglobulin, CA19-9, TPA, and/or in combination with the known protein(s) for the variant marker as described herein.

With regard to ovarian cancer, the polypeptides and/or polynucleotide of cluster HSCP2 of the present invention can be used in the diagnosis, treatment or prognostic assessment of invasive or metastatic ovarian cancer; correlating stage and malignant potential; identification of a metastasis of unknown origin which originated from a primary ovarian cancer; differential diagnosis between benign and malignant ovarian cysts; diagnosing a cause of infertility, for example differential diagnosis of various causes thereof; detecting of one or more non-ovarian cancer conditions that may elevate serum levels of ovary related markers, including but not limited to: cancers of the endometrium, cervix, fallopian tubes, pancreas, breast, lung and colon; nonmalignant conditions such as pregnancy, endometriosis, pelvic inflammatory disease and uterine fibroids; diagnosing conditions which have similar symptoms, signs and complications as ovarian cancer and where the differential diagnosis between them and ovarian cancer is of clinical importance including but not limited to: non-malignant causes of pelvic mass, including, but not limited to: benign (functional) ovarian cyst, uterine fibroids, endometriosis, benign ovarian neoplasms and inflammatory bowel lesions; determining a cause of any condition suggestive of a malignant tumor including but not limited to anorexia, cachexia, weight loss, fever, hypercalcemia, skeletal or abdominal pain, paraneoplastic syndrome, or ascites.

The polypeptides and/or polynucleotides of cluster HSCP2 used in the diagnosis, treatment or prognostic assessment of ovarian cancer can be used alone or in combination with one or more polypeptides and/or polynucleotides of this invention, and/or in combination with known markers for ovarian cancer, including but not limited to CEA, CA125 (Mucin 16), CA72-4TAG, CA-50, CA 54-61, CA-195 and CA 19-9 in combination with CA-125, and/or in combination with the known protein(s) associated with the indicated polypeptide or polynucleotide, as described herein.

According to one embodiment, the present invention provides a method for screening for a renal disease, and/or condition in a subject, comprising (a) obtaining a sample from the subject and (b) detecting in the sample at least one polynucleotide or at least one polypeptide being a member of a cluster selected from the group consisting of cluster AA340453, cluster AA703666, cluster AI590292, or cluster HUMUMOD. According to one embodiment, the presence of the polynucleotide or polypeptide in the sample is indicative of the presence of the disease and/or its severity and/or its progress. According to another embodiment, a change in the level of the polynucleotide or polypeptide in the sample compared to its level in a sample obtained from a healthy subject is indicative of the presence of the disease and/or its severity. According to another embodiment, a change in the level of the polynucleotide or polypeptide in the sample compared to its level in a sample previously obtained from said subject is indicative of the presence of the disease, its severity and/or the progress of the disease. According to currently preferred embodiments, the sample is a serum sample.

These markers are specifically released to the bloodstream under conditions of kidney-related diseases. The method of the present invention identifies clusters (genes) which are characterized in that the transcripts are differentially expressed in kidney tissue compared with other normal tissues. In acute conditions under which kidney tissue experiences damage (with or without necrosis), intracellular proteins that are not normally secreted can leak through the cell membrane to the extracellular space. In addition, secretion of soluble protein can be also altered in pathological conditions of kidney. Therefore, kidney tissue differentially expressed proteins, as through analysis of EST expression, are potential kidney damage markers whereas the damage is acute or chronic.

With regard to renal disease, and/or condition, the polypeptides and/or polynucleotides of this invention may be used for the diagnosis, treatment selection and monitoring, or assessment of prognosis of any type of renal disease, and/or condition including but not limited to any type of renal damage; renal cancer, including but not limited to renal cell carcinoma; polycystic kidney disease; Diabetes induced nephropathy; Chronic Kidney Disease; Total Kidney Failure; Autoimmune nephropathy, including but not limited to Systemic lupus erythematosus (SLE), Goodpasture's syndrome, IgA nephropathy; Hereditary Nephritis (Alport Syndrome); Infection-related Glomerular Disease, including but not limited to Acute post-streptococcal glomerulonephritis (PSGN), Bacterial endocarditis, HIV; Glomerulosclerosis; Focal segmental glomerulosclerosis (FSGS); Membranous nephropathy; Minimal change disease (MCD).

In some embodiments, the polypeptides/polynucleotides of this invention may be used for the diagnosis, treatment selection and monitoring, or assessment of prognosis of any type of renal disease, and/or condition in conjunction with other screening procedures and/or other markers, including but not limited to urinary protein, creatinine or creatinine clearance, and/or markers used for the diagnosis or assessment of prognosis of renal cancer, specifically of renal cell carcinoma, including but not limited to vascular endothelial growth factor, interleukin-12, the soluble interleukin-2 receptor, intercellular adhesion molecule-1, human chorionic gonadotropin beta, insulin-like growth factor-1 receptor, Carbonic anhydrase 9 (CA 9), endostatin, Thymidine phosphorylase or combinations thereof.

According to certain embodiments, a combination of anyone of the polynucleotides or polypeptides markers of the present invention with another marker can be used for determining a ratio between a quantitative or semi-quantitative measurement of any marker described herein to any other marker described herein, and/or any other known marker, and/or any other marker. With regard to such a ratio between any marker described herein (or a combination thereof) and a known marker, the known marker preferably comprises the “known protein” as described in greater detail below with regard to each cluster or gene.

It is to be understood that any polynucleotide or polypeptide of this invention may be useful as a marker for a disease, disorder or condition, and such use is to be considered a part of this invention.

According to certain embodiments, detecting the expression of a polynucleotide or polypeptide according to the teaching of the present invention is performed by employing a NAT-based technology (optionally by employing at least one nucleotide probe or primer), or by employing an immunoassay (optionally by employing an antibody according to any of the embodiments described herein), respectively.

In some embodiments, this invention provides a method for screening for a disease in a subject, comprising detecting in the subject or in a sample obtained from said subject at least one polypeptide or polynucleotide selected from the group consisting of:
a. a polypeptide having an amino acid sequence as set forth in any one of SEQ. ID NOs: 91-96, 144-155, 180-183, 246-249, 310-322, 365-370, or a homologue or a fragment thereof;
b. a polypeptide comprising a bridge, edge portion, tail, or head portion, wherein the polypeptide has an amino acid sequence as set forth in any one of SEQ. ID NOs: 372-437, or a homologue or a fragment thereof;
c. a polynucleotide having a nucleic acid sequence as set forth in any one of SEQ. ID NOs: 37-42, 105-119, 165-167, 190-193, 259-271, 329-334, or a homologue or a fragment thereof;
d. a polynucleotide comprising a node having a nucleic acid sequence as set forth in any one of SEQ. ID NOs: 43-88, 120-142, 168-177, 194-241, 272-307, 335-360;
e. an oligonucleotide having a nucleic acid sequence as set forth in SEQ. ID NOs: 83, 99, 102, 158, 161, 164, 186, 189, 252, 255, 258, 325, 328, 440.

According to one embodiment, detecting the presence of the polypeptide or polynucleotide is indicative of the presence of the disease and/or its severity and/or its progress. According to another embodiment, a change in the expression and/or the level of the polynucleotide or polypeptide compared to its expression and/or level in a healthy subject or a sample obtained therefrom is indicative of the presence of the disease and/or its severity and/or its progress. According to a further embodiment, a change in the expression and/or level of the polynucleotide or polypeptide compared to its level and/or expression in said subject or in a sample obtained therefrom at earlier stage is indicative of the progress of the disease. According to still further embodiment, detecting the presence and/or relative change in the expression and/or level of the polynucleotide or polypeptide is useful for selecting a treatment and/or monitoring a treatment of the disease.

According to one embodiment, detecting a polynucleotide of the invention comprises employing a primer pair, comprising a pair of isolated oligonucleotides capable of specifically hybridizing to at least a portion of a polynucleotide having a nucleic acid sequence as set forth in SEQ. ID NOs: 83, 99, 102, 158, 161, 164, 186, 189, 252, 255, 258, 325, 328, 440 or polynucleotides homologous thereto.

According to another embodiment, detecting a polynucleotide of the invention comprises employing a primer pair, comprising a pair of isolated oligonucleotides as set forth in SEQ. ID NOs:97-98, 100-101, 156-157, 159-160, 162-163, 184-185, 187-188, 250-251, 253-254, 256-257, 323-324, 326-327, 438-439.

According to further embodiment, detecting a polypeptide of the invention comprises employing an antibody capable of specifically binding to at least one epitope of a polypeptide comprising an amino acid sequence as set forth in any one of SEQ. ID NOs: 91-96, 144-155, 180-183, 246-249, 310-322, 365-370, 372-437.

In some embodiments, a method of this invention may make use of a polynucleotide, polypeptide, vector, antibody, biomarker, or combination thereof, as described herein, including any embodiments thereof.

In some embodiments, the methods of this invention are conducted on a whole body. According to other embodiments, the methods of the present invention are conducted with a sample isolated from a subject having, predisposed to, or suspected of having the disease, disorder or condition. According to certain embodiments, the sample is a cell or tissue or a body fluid sample. In some embodiments, the methods are directed to the monitoring of disease progression and/or treatment efficacy and/or relapse of the indicated disease, disorder or condition.

In another embodiment, this invention provides methods for the selection of a particular therapy, or optimization of a given therapy for a disease, disorder or condition, the method comprising quantitatively and/or qualitatively determining or assessing expression of the polypeptides and/or polynucleotides, whereby differences in expression from an index sample, or a sample taken from a subject prior to the initiation of the therapy, or during the course of therapy, is indicative of the efficacy, or optimal activity of the therapy.

According to still further aspect, the present invention provides a method for detecting a splice variant nucleic acid sequence in a biological sample, comprising: hybridizing the isolated splice variant nucleic acid molecules or oligonucleotide fragments thereof of at least about 12 nucleotides to a nucleic acid material of the biological sample and detecting a hybridization complex; wherein the presence of the hybridization complex correlates with the presence of said splice variant nucleic acid sequence in the biological sample.

The nucleic acid sequences and/or amino acid sequences shown herein as embodiments of the present invention relate, in some embodiments, to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that the terms “oligonucleotide” and “polynucleotide” and “nucleic acid molecule”, or “peptide” and “polypeptide” and “protein”, may optionally be used interchangeably.

All technical and scientific terms used herein should be understood to have the meaning commonly understood by a person skilled in the art to which this invention belongs, as well as any other specified description. The following references provide one of skill in the art with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a schematic description of the cancer biomarker selection engine.

FIG. 2 shows a schematic presentation of the oligonucleotide based microarray fabrication.

FIG. 3 shows a schematic summary of the oligonucleotide based microarray experimental flow.

FIG. 4 shows a schematic summary of quantitative real-time PCR analysis.

FIG. 5 is a histogram showing relative expression of the Homo sapiens cadherin 16, KSP-cadherin (CDH16) transcripts which are detectable by amplicon as depicted in sequence name AA340453_seg24F2R2 (SEQ ID NO:99) in kidney tissue samples as opposed to other tissues.

FIG. 6 is a histogram showing relative expression of the Homo sapiens cadherin 16, KSP-cadherin (CDH16) AA340453 transcripts which are detectable by amplicon as depicted in sequence name AA340453_seg44 (SEQ ID NO:102) in kidney tissue samples as opposed to other tissues.

FIG. 7 is a histogram showing relative expression of the Homo sapiens cadherin 16, KSP-cadherin (CDH16) AA340453 transcripts which are detectable by amplicon as depicted in sequence name AA340453_seg55WT (SEQ ID NO:83) in kidney tissue samples as opposed to other tissues.

FIG. 8 is a histogram showing relative expression of the Homo sapiens aldehyde reductase (aldose reductase) like 6 (ALDRL6) AA703666 transcripts which are detectable by amplicon as depicted in sequence name AA703666_junc12-17 (SEQ ID NO:158) in kidney tissue samples as opposed to other tissues.

FIG. 9 is a histogram showing relative expression of the Homo sapiens aldehyde reductase (aldose reductase) like 6 (ALDRL6) AA703666 transcripts which are detectable by amplicon as depicted in sequence name AA703666_seg9WT (SEQ ID NO:161) in kidney tissue samples as opposed to other tissues.

FIG. 10 is a histogram showing relative expression of the Homo sapiens aldehyde reductase (aldose reductase) like 6 (ALDRL6) AA703666 transcripts which are detectable by amplicon as depicted in sequence name AA703666_seg18 (SEQ ID NO:164) in kidney tissue samples as opposed to other tissues.

FIG. 11 is a histogram showing relative expression of the Homo sapiens nephrosis 2, idiopathic, steroid-resistant (podocin) (NPHS2) AI590292 transcripts which are detectable by amplicon as depicted in sequence name AI590292_junc2-6 (SEQ ID NO:186) in kidney tissue samples as opposed to other tissues.

FIG. 12 is a histogram showing relative expression of the Homo sapiens nephrosis 2, idiopathic, steroid-resistant (podocin) (NPHS2) AI590292 transcripts which are detectable by amplicon as depicted in sequence name AI590292_seg4WT (SEQ ID NO:189) in kidney tissue samples as opposed to other tissues.

FIG. 13 is a histogram showing relative expression of the Homo sapiens uromodulin (uromucoid, Tamm-Horsfall glycoprotein) (UMOD) HUMUMOD transcripts which are detectable by amplicon as depicted in sequence name HUMUMOD_seg47-48 (SEQ ID NO:252) in kidney tissue samples as opposed to other tissues.

FIG. 14 is a histogram showing relative expression of the Homo sapiens uromodulin (uromucoid, Tamm-Horsfall glycoprotein) (UMOD) HUMUMOD transcripts which are detectable by amplicon as depicted in sequence name HUMUMOD_seg56 (SEQ ID NO:255) in kidney tissue samples as opposed to other tissues.

FIG. 15 is a histogram showing relative expression of the Homo sapiens uromodulin (uromucoid, Tamm-Horsfall glycoprotein) (UMOD) HUMUMOD transcripts which are detectable by amplicon as depicted in sequence name HUMUMOD_seg61WT (SEQ ID NO:258) in kidney tissue samples as opposed to other tissues.

FIG. 16 is a histogram showing that cluster HSCP2 is overexpressed (at least at a minimum level) in the following pathological conditions: ovarian carcinoma and kidney malignant tumors.

FIG. 17 is a histogram showing over expression of the Homo sapiens ceruloplasmin (ferroxidase) (CP) HSCP2 transcripts which are detectable by amplicon as depicted in sequence name HSCP2_junc0-13 (SEQ ID NO:325) in cancerous Lung samples relative to the normal samples.

FIGS. 18a-b are histograms showing expression of Homo sapiens ceruloplasmin (ferroxidase) (CP) HSCP2 transcripts which are detectable by amplicon as depicted in sequence name HSCP2_junc0-13 (SEQ ID NO:325) in different normal tissues, relative to median of the lung samples (FIG. 18a) or relative to median of the ovary samples (FIG. 18b).

FIG. 19 is a histogram showing over expression of the Homo sapiens ceruloplasmin (ferroxidase) (CP) HSCP2 transcripts which are detectable by amplicon as depicted in sequence name HSCP2_junc0-13 (SEQ ID NO:325) in cancerous Ovary samples relative to the normal samples.

FIG. 20 is a histogram showing over expression of the Homo sapiens ceruloplasmin (ferroxidase) (CP) HSCP2 transcripts which are detectable by amplicon as depicted in sequence name HSCP2_seg3WT (SEQ ID NO:328) in cancerous Lung samples relative to the normal samples.

FIGS. 21a-b are histograms showing expression of Homo sapiens ceruloplasmin (ferroxidase) (CP) HSCP2 transcripts which are detectable by amplicon as depicted in sequence name seg3WT (SEQ ID NO:328) in different normal tissues, relative to median of the lung samples (FIG. 21a) or relative to median of the ovary samples (FIG. 21b).

FIG. 22 is a histogram showing over expression of the Homo sapiens ceruloplasmin (ferroxidase) (CP) HSCP2 transcripts which are detectable by amplicon as depicted in sequence name seg3WT (SEQ ID NO:328) in cancerous Ovary samples relative to the normal samples.

FIG. 23 is a histogram showing over expression of the Homo sapiens aldehyde reductase like 6 (ALDRL6) AA7030666 transcripts which are detectable by amplicon as depicted in sequence name AA7030666_junc4-7F2R2 (SEQ ID NO:440) specifically in kidney tissue.

### DESCRIPTION OF PREFERRED EMBODIMENTS

The present invention provides polynucleotides, polypeptides, particularly variants of known proteins, and uses thereof, particularly as diagnostic markers.

In some embodiments, the polypeptides and polynucleotides of the present invention are useful as diagnostic markers for certain diseases, and as such the term “marker-detectable” or “variant-detectable” with regard to a disease is to be understood as encompassing use of the described polynucleotides and/or polypeptides for diagnosis.

In some embodiments, certain diseases are associated with differential expression, qualitatively or quantitatively, of the polynucleotides and polypeptides of this invention. Assessment of such expression, in turn, can therefore serve as a marker for a particular disease state, susceptibility to a disease, pathogenesis, etc., including any desired disease-specific event, whose analysis is useful, as will be appreciated by one skilled in the art. In one embodiment, such use as a marker is also referred to herein as the polynucleotides and polypeptides being “variant disease markers”.

The markers of the present invention, alone or in combination, can be used for prognosis, prediction, screening, early diagnosis, staging, therapy selection and treatment monitoring of a marker-detectable disease. For example, optionally and preferably, these markers may be used for staging the disease in patient (for example if the disease features cancer) and/or monitoring the progression of the disease. Furthermore, the markers of the present invention, alone or in combination, can be used for detection of the source of metastasis found in anatomical places other than the originating tissue, again in the example of cancer. Also, one or more of the markers may optionally be used in combination with one or more other disease markers (other than those described herein).

Biomolecular sequences (amino acid and/or nucleic acid sequences) uncovered using the methodology of the present invention and described herein can be efficiently utilized as tissue or pathological markers and/or as drugs or drug targets for treating or preventing a disease.

In some embodiments, these markers are specifically released to the bloodstream under conditions of a particular disease, and/or are otherwise expressed at a much higher level and/or specifically expressed in tissue or cells afflicted with or demonstrating the disease. The measurement of these markers, alone or in combination, in patient samples provides information that the diagnostician can correlate with a probable diagnosis of a particular disease and/or a condition that is indicative of a higher risk for a particular disease.

The present invention provides, in some embodiments, diagnostic assays for a marker-detectable disease and/or an indicative condition, and methods of use of such markers for detection of marker-detectable disease and/or an indicative condition, for example in a sample taken from a subject (patient), which in some embodiments, is a blood sample.

Some embodiments of this invention have been exemplified herein wherein cellular localization was determined according to four different software programs: (i) tmhmm (from Center for Biological Sequence Analysis, Technical University of Denmark DTU, http://www.cbs.dtu.dk/services/TMHMM/TMHMM2.0b.guide.php) or (ii) tmpred (from EMBnet, maintained by the ISREC Bionformatics group and the LICR Information Technology Office, Ludwig Institute for Cancer Research, Swiss Institute of Bioinformatics, http://www.ch.embnet.org/software/TMPRED_form.html) for transmembrane region prediction; (iii) signalp_hmm or (iv) signalp_nn (both from Center for Biological Sequence Analysis, Technical University of Denmark DTU, http://www.cbs.dtu.dk/services/SignalP/background/prediction.php) for signal peptide prediction. The terms “signalp_hmm” and “signalp_nn” refer to two modes of operation for the program SignalP:hmm refers to Hidden Markov Model, while nn refers to neural networks. Localization was also determined through manual inspection of known protein localization and/or gene structure, and the use of heuristics by the individual inventor. In some cases for the manual inspection of cellular localization prediction inventors used the ProLoc computational platform [Einat Hazkani-Covo, Erez Levanon, Galit Rotman, Dan Graur and Amit Novik; (2004) “Evolution of multicellularity in metazoa: comparative analysis of the subcellular localization of proteins in Saccharomyces, Drosophila and Caenorhabditis.” Cell Biology International 2004; 28(3):171-8.], which predicts protein localization based on various parameters including, protein domains (e.g., prediction of trans-membranous regions and localization thereof within the protein), pI, protein length, amino acid composition, homology to pre-annotated proteins, recognition of sequence patterns which direct the protein to a certain organelle (such as, nuclear localization signal, NLS, mitochondria localization signal), signal peptide and anchor modeling and using unique domains from Pfam that are specific to a single compartment.

Information is given in the text with regard to SNPs (single nucleotide polymorphisms). A description of the abbreviations is as follows. “T−>C”, for example, means that the SNP results in a change at the position given in the table from T to C. Similarly, “M−>Q”, for example, means that the SNP has caused a change in the corresponding amino acid sequence, from methionine (M) to glutamine (Q). If, in place of a letter at the right hand side for the nucleotide sequence SNP, there is a space, it indicates that a frame shift has occurred. A frame shift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows construction of links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number. In the table of the amino acid mutations of the wild type proteins of the selected splice variants of the invention, the header of the first column is “SNP position(s) on amino acid sequence”, representing a position of a known mutation on amino acid sequence. SNPs may optionally be used as diagnostic markers according to the present invention, alone or in combination with one or more other SNPs and/or any other diagnostic marker. Preferred embodiments of the present invention comprise such SNPs, including but not limited to novel SNPs on the known (WT or wild type) protein sequences given below, as well as novel nucleic acid and/or amino acid sequences formed through such SNPs, and/or any SNP on a variant amino acid and/or nucleic acid sequence described herein.

Information given in the text with regard to the Homology to the known proteins was determined by Smith-Waterman version 5.1.2 using special (non default) parameters as follows:

model=sw.model

GAPEXT=0

GAPOP=100.0
MATRIX=blosum100

Information is given with regard to overexpression of a cluster in cancer based on ESTs. A key to the p values with regard to the analysis of such overexpression is as follows:
Library-based statistics: P-value without including the level of expression in cell-lines (P1)
Library based statistics: P-value including the level of expression in cell-lines (P2)
EST clone statistics: P-value without including the level of expression in cell-lines (SP1)
EST clone statistics: predicted overexpression ratio without including the level of expression in cell-lines (R3)
EST clone statistics: P-value including the level of expression in cell-lines (SP2)
EST clone statistics: predicted overexpression ratio including the level of expression in cell-lines (R4)

Library-based statistics refer to statistics over an entire library, while EST clone statistics refer to expression only for ESTs from a particular tissue or cancer.

Some embodiments of this invention have been exemplified herein wherein overexpression of a cluster in cancer was a determination based on microarray use. As a microarray reference, in the specific segment paragraphs, the unabbreviated tissue name was used as the reference to the type of chip for which expression was measured. There are two types of microarray results: those from microarrays prepared according to a design by the present inventors, for which the microarray fabrication procedure is described in detail in Materials and Experimental Procedures section herein; and those results from microarrays using Affymetrix technology. As a microarray reference, in the specific segment paragraphs, the unabbreviated tissue name was used as the reference to the type of chip for which expression was measured. For microarrays prepared according to a design by the present inventors, the probe name begins with the name of the cluster (gene), followed by an identifying number.

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgul 33.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 April 09).

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgu133.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgul33plus.affx). The probe names follow the Affymetrix naming convention. The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 April 09).

The following list of abbreviations for tissues was used in the TAA histograms. The term “TAA” stands for “Tumor Associated Antigen”, and the TAA histograms, given in the text, represent the cancerous tissue expression pattern as predicted by the biomarkers selection engine, as described in detail in examples 1-5 below (the first word is the abbreviation while the second word is the full name):

(“BONE”, “bone”);
(“COL”, “colon”);
(“EPI”, “epithelial”);
(“GEN”, “general”);
(“LIVER”, “liver”);
(“LUN”, “lung”);
(“LYMPH”, “lymph nodes”);
(“MARROW”, “bone marrow”);
(“OVA”, “ovary”);
(“PANCREAS”, “pancreas”);
(“PRO”, “prostate”);
(“STOMACH”, “stomach”);
(“TCELL”, “T cells”);

### (“THYROID”, “Thyroid”);

(“MAM”, “breast”);
(“BRAIN”, “brain”);
(“UTERUS”, “uterus”);
(“SKIN”, “skin”);
(“KIDNEY”, “kidney”);
(“MUSCLE”, “muscle”);
(“ADREN”, “adrenal”);
(“HEAD”, “head and neck”);
(“BLADDER”, “bladder”);

It should be noted that the terms “segment”, “seg” and “node” (abbreviated as “N” in the names of nodes) are used interchangeably in reference to nucleic acid sequences of the present invention, they refer to portions of nucleic acid sequences that were shown to have one or more properties as described herein. They are also the building blocks that were used to construct complete nucleic acid sequences as described in greater detail elsewhere herein. Optionally and preferably, they are examples of oligonucleotides which are embodiments of the present invention, for example as amplicons, hybridization units and/or from which primers and/or complementary oligonucleotides may optionally be derived, and/or for any other use.

In some embodiments, the phrase “disease” refers to its commonly understood meaning, and includes, inter alia, any type of pathology and/or damage, including both chronic and acute damage, as well as a progress from acute to chronic damage.

In some embodiments, the phrase “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

In some embodiments, the term “polypeptide” is to be understood to refer to a molecule comprising from at least 2 to several thousand or more amino acids. The term “polypeptide” is to be understood to include, inter alia, native peptides (either degradation products, synthetically synthesized peptides or recombinant peptides), peptidomimetics, such as peptoids and semipeptoids or peptide analogs, which may comprise, for example, any desirable modification, including, inter alia, modifications rendering the peptides more stable while in a body or more capable of penetrating into cells, or others as will be appreciated by one skilled in the art. Such modifications include, but are not limited to N terminus modification, C terminus modification, peptide bond modification, backbone modifications, residue modification, or others. Inclusion of such peptides within the polypeptides of this invention may produce a polypeptide sharing identity with the polypeptides described herein, for example, those provided in the sequence listing.

In some embodiments, the phrase “differentially present” refers to differences in the quantity or quality of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the peptide and polypeptide, may optionally be used interchangeably other sample. It should be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

In some embodiments, the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In some embodiments, the phrase “qualitative” when in reference to differences in expression levels of a polynucleotide, polypeptide or cluster as described herein, refers to the presence versus absence of expression, or in some embodiments, the temporal regulation of expression, or in some embodiments, the timing of expression, or in some embodiments, the variant expressed, or in some embodiments, any post-translational modifications to the expressed molecule, and others, as will be appreciated by one skilled in the art. In some embodiments, the phrase “quantitative” when in reference to differences in expression levels of a polynucleotide, polypeptide or cluster as described herein, refers to absolute differences in quantity of expression, as determined by any means, known in the art, or in other embodiments, relative differences, which may be statistically significant, or in some embodiments, when viewed as a whole or over a prolonged period of time, etc., indicate a trend in terms of differences in expression.

In some embodiments, the term “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

In some embodiments, the term “level” refers to expression levels of RNA and/or protein or to DNA copy number of a marker of the present invention.

Typically the level of the marker in a biological sample obtained from the subject is different (i.e., increased or decreased) from the level of the same variant in a similar sample obtained from a healthy individual (examples of biological samples are described herein).

Numerous well known tissue or fluid collection methods can be utilized to collect the biological sample from the subject in order to determine the level of DNA, RNA and/or polypeptide of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

In some embodiments, the term “test amount” of a marker refers to an amount of a marker in a subject's sample that is consistent with a diagnosis of a particular disease or condition. A test amount can be either in absolute amount (e.g., microgram/ml) or a relative amount (e.g., relative intensity of signals).

In some embodiments, the term “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/ml) or a relative amount (e.g., relative intensity of signals).

In some embodiments, the term “detect” refers to identifying the presence, absence or amount of the object to be detected.

In some embodiments, the term “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavadin, dioxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” or “specifically interacts or binds” when referring to a protein or peptide (or other epitope), refers, in some embodiments, to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, the present invention relates to bridges, tails, heads and/or insertions, and/or analogs, homologs and derivatives of such peptides. Such bridges, tails, heads and/or insertions are described in greater detail below with regard to the Examples.

In some embodiments, the term “tail” refers to a peptide sequence at the end of an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding known protein, while at least a second portion of the variant comprises the tail.

In some embodiments, the term “head” refers to a peptide sequence at the beginning of an amino acid sequence that is unique to a splice variant according to the present invention. Therefore, a splice variant having such a head may optionally be considered as a chimera, in that at least a first portion of the splice variant comprises the head, while at least a second portion is typically highly homologous (often 100% identical) to a portion of the corresponding known protein.

In some embodiments, the term “an edge portion” refers to a connection between two portions of a splice variant according to the present invention that were not joined in the wild type or known protein. An edge may optionally arise due to a join between the above “known protein” portion of a variant and the tail, for example, and/or may occur if an internal portion of the wild type sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

In some embodiments, a bridge between a tail or a head or a unique insertion, and a “known protein” portion of a variant, comprises at least about 10 amino acids, or in some embodiments at least about 20 amino acids, or in some embodiments at least about 30 amino acids, or in some embodiments at least about 40 amino acids, in which at least one amino acid is from the tail/head/insertion and at least one amino acid is from the “known protein” portion of a variant. In some embodiments, the bridge may comprise any number of amino acids from about 10 to about 40 amino acids (for example, 10, 11, 12, 13 . . . 37, 38, 39, 40 amino acids in length, or any number in between).

It should be noted that a bridge cannot be extended beyond the length of the sequence in either direction, and it should be assumed that every bridge description is to be read in such manner that the bridge length does not extend beyond the sequence itself.

Furthermore, bridges are described with regard to a sliding window in certain contexts below. For example, certain descriptions of the bridges feature the following format: a bridge between two edges (in which a portion of the known protein is not present in the variant) may optionally be described as follows: a bridge portion of CONTIG-NAME_P1 (representing the name of the protein), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise XX (2 amino acids in the center of the bridge, one from each end of the edge), having a structure as follows (numbering according to the sequence of CONTIG-NAME_P1): a sequence starting from any of amino acid numbers 49-x to 49 (for example); and ending at any of amino acid numbers 50+((n−2)−x) (for example), in which x varies from 0 to n−2. In this example, it should also be read as including bridges in which n is any number of amino acids between 10-50 amino acids in length. Furthermore, the bridge polypeptide cannot extend beyond the sequence, so it should be read such that 49-x (for example) is not less than 1, nor 50+((n−2)−x) (for example) greater than the total sequence length.

In another embodiment, this invention provides isolated nucleic acid molecules, which in some embodiments encode for splice variants, having a nucleotide sequence as set forth in any one of the sequences listed herein, being homologous to such sequences, at a percent as described herein, or a sequence complementary thereto. In another embodiment, this invention provides an oligonucleotide of at least about 12 nucleotides, which specifically hybridizes with the nucleic acid molecules of this invention. In another embodiment, this invention provides vectors, cells, liposomes and compositions comprising the isolated nucleic acids or polypeptides of this invention, as appropriate.

In another embodiment, this invention provides antibodies specifically recognizing the splice variants and polypeptide fragments thereof of this invention. Preferably such antibodies differentially recognize splice variants of the present invention but do not recognize a corresponding known protein (such known proteins are discussed with regard to their splice variants in the Examples below).

In another embodiment, this invention provides a method for detecting a splice variant according to the present invention in a biological sample, comprising: contacting a biological sample with an antibody specifically recognizing a splice variant according to the present invention under conditions whereby the antibody specifically interacts with the splice variant in the biological sample but do not recognize known corresponding proteins (wherein the known protein is discussed with regard to its splice variant(s) in the Examples below), and detecting said interaction; wherein the presence of an interaction correlates with the presence of a splice variant in the biological sample.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

According to the present invention, the splice variants described herein are non-limiting examples of markers for diagnosing marker-detectable disease and/or an indicative condition. Each splice variant marker of the present invention can be used alone or in combination, for various uses, including but not limited to, prognosis, prediction, screening, early diagnosis, determination of progression, therapy selection and treatment monitoring of marker-detectable disease and/or an indicative condition, including a transition from an indicative condition to marker-detectable disease.

According to optional but preferred embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

In some embodiments of the present invention, there are provided of methods, uses, devices and assays for the diagnosis of a disease or condition. Optionally a plurality of biomarkers (or markers) may be used with the present invention. The plurality of markers may optionally include a plurality of markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlates with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers are outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. For example, with stroke as a non-limiting example of a disease or condition, certain markers in a panel may be commonly used to diagnose the existence of a stroke, while other members of the panel may indicate if an acute stroke has occurred, while still other members of the panel may indicate if a non-acute stroke has occurred. Markers may also be commonly used for multiple purposes by, for example, applying a different threshold or a different weighting factor to the marker for the different purpose(s). For example, again with stroke as a non-limiting example of a disease or condition, a marker at one concentration or weighting may be used, alone or as part of a larger panel, to indicate if an acute stroke has occurred, and the same marker at a different concentration or weighting may be used, alone or as part of a larger panel, to indicate if a non-acute stroke has occurred.

In one embodiment, the panels comprise markers for the following purposes: diagnosis of a disease; diagnosis of disease and indication if the disease is in an acute phase and/or if an acute attack of the disease has occurred (for example for CVS, heart disease, stroke and/or cerebrovascular accident); diagnosis of disease and indication if the disease is in a non-acute phase and/or if a non-acute attack of the disease has occurred (for example for CVS, heart disease, stroke and/or cerebrovascular accident); indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art (see for example Hanley et al., Radiology 143: 29-36 (1982), incorporated by reference as if fully set forth herein).

One or more markers may lack diagnostic or prognostic value when considered alone, but when used as part of a panel, such markers may be of great value in determining a particular diagnosis/prognosis. In preferred embodiments, particular thresholds for one or more markers in a panel are not relied upon to determine if a profile of marker levels obtained from a subject are indicative of a particular diagnosis/prognosis. Rather, the present invention may utilize an evaluation of the entire marker profile by plotting ROC curves for the sensitivity of a particular panel of markers versus 1-(specificity) for the panel at various cutoffs. In these methods, a profile of marker measurements from a subject is considered together to provide a global probability (expressed either as a numeric score or as a percentage risk) that an individual has had a disease, is at risk for developing such a disease, optionally the type of disease which the individual has had or is at risk for, and so forth etc. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In some embodiments, markers and/or marker panels are selected to exhibit at least 70% sensitivity, more preferably at least 80% sensitivity, even more preferably at least 85% sensitivity, still more preferably at least 90% sensitivity, and most preferably at least 95% sensitivity, combined with at least 70% specificity, more preferably at least 80% specificity, even more preferably at least 85% specificity, still more preferably at least 90% specificity, and most preferably at least 95% specificity. In particularly preferred embodiments, both the sensitivity and specificity are at least 75%, more preferably at least 80%, even more preferably at least 85%, still more preferably at least 90%, and most preferably at least 95%. Sensitivity and/or specificity may optionally be determined as described above, with regard to the construction of ROC graphs and so forth, for example.

According to some embodiments of the present invention, individual markers and/or combinations (panels) of markers may optionally be used for diagnosis of time of onset of a disease or condition. Such diagnosis may optionally be useful for a wide variety of conditions, preferably including those conditions with an abrupt onset.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In preferred embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

The skilled artisan will understand that associating a prognostic indicator with a predisposition to an adverse outcome is a statistical analysis. For example, a marker level of greater than 80 pg/mL may signal that a patient is more likely to suffer from an adverse outcome than patients with a level less than or equal to 80 pg/mL, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described hereinafter.

In other embodiments, a threshold degree of change in the level of a prognostic or diagnostic indicator can be established, and the degree of change in the level of the indicator in a patient sample can simply be compared to the threshold degree of change in the level. A preferred threshold change in the level for markers of the invention is about 5%, about 10%, about 15%, about 20%, about 25%, about 30%, about 50%, about 75%, about 100%, and about 150%. The term “about” in this context refers to +/−10%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

Exemplary, non-limiting methods and systems for identification of suitable biomarkers for marker panels are now described. Methods and systems for the identification of a one or more markers for the diagnosis, and in particular for the differential diagnosis, of disease have been described previously. Suitable methods for identifying markers useful for the diagnosis of disease states are described in detail in U.S. patent application no. 2004-0126767, entitled METHOD AND SYSTEM FOR DISEASE DETECTION USING MARKER COMBINATIONS, filed Dec. 27, 2002, hereby incorporated by reference in its entirety as if fully set forth herein. One skilled in the art will also recognize that univariate analysis of markers can be performed and the data from the univariate analyses of multiple markers can be combined to form panels of markers to differentiate different disease conditions.

In developing a panel of markers useful in diagnosis, data for a number of potential markers may be obtained from a group of subjects by testing for the presence or level of certain markers. The group of subjects is divided into two sets, and preferably the first set and the second set each have an approximately equal number of subjects. The first set includes subjects who have been confirmed as having a disease or, more generally, being in a first condition state. For example, this first set of patients may be those that have recently had a disease and/or a particular type of the disease. The confirmation of this condition state may be made through more rigorous and/or expensive testing, preferably according to a previously defined diagnostic standard. Hereinafter, subjects in this first set will be referred to as “diseased”.

The second set of subjects is simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects.

The data obtained from subjects in these sets includes levels of a plurality of markers. Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve as described above.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself. For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In a preferred embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides: In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels which require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

Individual panel response values may also be used as markers in the methods described herein. For example, a panel may be constructed from a plurality of markers, and each marker of the panel may be described by a function and a weighting factor to be applied to that marker (as determined by the methods described above). Each individual marker level is determined for a sample to be tested, and that level is applied to the predetermined function and weighting factor for that particular marker to arrive at a sample value for that marker. The sample values for each marker are added together to arrive at the panel response for that particular sample to be tested. For a “diseased” and “non-diseased” group of patients, the resulting panel responses may be treated as if they were just levels of another disease marker.

Measures of test accuracy may be obtained as described in Fischer et al., Intensive Care Med. 29: 1043-51, 2003 (hereby incorporated by reference as if fully set forth herein), and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures: at least 75% sensitivity, combined with at least 75% specificity; ROC curve area of at least 0.7, more preferably at least 0.8, even more preferably at least 0.9, and most preferably at least 0.95; and/or a positive likelihood ratio (calculated as sensitivity/(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

According to other preferred embodiments of the present invention, a splice variant protein or a fragment thereof, or a splice variant nucleic acid sequence or a fragment thereof, may be featured as a biomarker for detecting marker-detectable disease and/or an indicative condition, such that a biomarker may optionally comprise any of the above.

According to still other preferred embodiments, the present invention optionally and preferably encompasses any amino acid sequence or fragment thereof encoded by a nucleic acid sequence corresponding to a splice variant protein as described herein. Any oligopeptide or peptide relating to such an amino acid sequence or fragment thereof may optionally also (additionally or alternatively) be used as a biomarker, including but not limited to the unique amino acid sequences of these proteins that are depicted as tails, heads, insertions, edges or bridges. The present invention also optionally encompasses antibodies capable of recognizing, and/or being elicited by, such oligopeptides or peptides.

The present invention also optionally and preferably encompasses any nucleic acid sequence or fragment thereof, or amino acid sequence or fragment thereof, corresponding to a splice variant of the present invention as described above, optionally for any application.

Non-limiting examples of methods or assays are described below.

The present invention also relates to kits based upon such diagnostic methods or assays.

### Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof, sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioetlier, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed Examples of cell type-specific and/or tissue-specific promoters include promoters such as albumin that is liver specific, lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., includingRetro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, Herpes simplex I, virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Variant Recombinant Expression Vectors and Host Cells

Another aspect of the invention pertains to vectors, preferably expression vectors, containing a nucleic acid encoding a variant protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., variant proteins, mutant forms of variant proteins, fusion proteins, etc.).

The recombinant expression vectors of the invention can be designed for production of variant proteins in prokaryotic or eukaryotic cells. For example, variant proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or carboxyl terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) and pTrcHis (Invitrogen Life Technologies) that fuse glutathione S-transferase (GST), maltose E binding protein, protein A or 6×His, respectively, to the target recombinant protein.

Examples of suitable inducible non-fusion E. coli expression vectors include pTrc (Amrann et al., (1988) Gene 69:301-315).

One strategy to maximize recombinant protein expression in E. coli is to express the protein in host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another optional strategy to solve codon bias is by using BL21-codon plus bacterial strains (Invitrogen) or Rosetta bacterial strain (Novagen), as these strains contain extra copies of rare E. coli tRNA genes.

In another embodiment, the expression vector encoding for the variant protein is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, variant protein can be produced in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for variant protein. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, variant protein can be produced in bacterial cells such as E. coli, insect cells, yeast or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, puromycin, blasticidin and methotrexate. Nucleic acids encoding a selectable marker can be introduced into a host cell on the same vector as that encoding variant protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) variant protein. Accordingly, the invention further provides methods for producing variant protein using the host cells of the invention. In one embodiment, the method comprises culturing the host cell of the present invention (into which a recombinant expression vector encoding variant protein has been introduced) in a suitable medium such that variant protein is produced. In another embodiment, the method further comprises isolating variant protein from the medium or the host cell.

For efficient production of the protein, it is preferable to place the nucleotide sequences encoding the variant protein under the control of expression control sequences optimized for expression in a desired host. For example, the sequences may include optimized transcriptional and/or translational regulatory sequences (such as altered Kozak sequences).

### Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ; (ii) hybridization solution of 6×SSC and 0.1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S, Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include ³H, ¹⁴C, ³²P, and ³⁵S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

### NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. NatI. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 220, or 1,048,576 copies of the starting material. If the reaction conditions reduce the mean efficiency to 85%, then the yield in those 20 cycles will be only 1.8520, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station. describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

### Amino Acid Sequences and Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include but are not limited to exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaba Young, Solid Phase Peptide Syntheses (2nd Ed., Pierce Chemical Company, 1984).

Synthetic polypeptides can optionally be purified by preparative high performance liquid chromatography [Creighton T. (1983) Proteins, structures and molecular principles. WH Freeman and Co. N.Y.], after which their composition can be confirmed via amino acid sequencing.

In cases where large amounts of a polypeptide are desired, it can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

The present invention also encompasses polypeptides encoded by the polynucleotide sequences of the present invention, as well as polypeptides according to the amino acid sequences described herein. The present invention also encompasses homologues of these polypeptides, such homologues can be at least 50%, at least 55%, at least 60%, at least 65%, at least 70%, at least 75%, at least 80%, at least 85%, at least 95% or more say 100% homologous to the amino acid sequences set forth below, as can be determined using BlastP software of the National Center of Biotechnology Information (NCBI) using default parameters, optionally and preferably including the following: filtering on (this option filters repetitive or low-complexity sequences from the query using the Seg (protein) program), scoring matrix is BLOSUM62 for proteins, word size is 3, E value is 10, gap costs are 11, 1 (initialization and extension), and number of alignments shown is 50. Preferably, nucleic acid sequence homology/identity is determined by using BlastN software of the National Center of Biotechnology Information (NCBI) using default parameters, which preferably include using the DUST filter program, and also preferably include having an E value of 10, filtering low complexity sequences and a word size of 11. Finally, the present invention also encompasses fragments of the above described polypeptides and polypeptides having mutations, such as deletions, insertions or substitutions of one or more amino acids, either naturally occurring or artificially induced, either randomly or in a targeted fashion.

It will be appreciated that peptides identified according the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Non-conventional or modified amino acids can be incorporated in the polypeptides of this invention as well, as will be known to one skilled in the art.

Since the peptides of the present invention are preferably utilized in diagnostics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclization does not severely interfere with peptide characteristics, cyclic forms of the peptide can also be utilized.

The peptides of present invention can be biochemically synthesized such as by using standard solid phase techniques. These methods include exclusive solid phase synthesis well known in the art, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Synthetic peptides can be purified by preparative high performance liquid chromatography and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and also as described above.

### Antibodies:

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Monoclonal antibody development may optionally be performed according to any method that is known in the art. The method described below is provided for the purposes of description only and is not meant to be limiting in any way.

### Antibody Engineering in Phage Display Libraries:

Antibodies of this invention may be prepared through the use of phage display libraries, as is known in the art, for example, as described in PCT Application No. WO 94/18219, U.S. Pat. No. 6,096,551, both of which are hereby fully incorporated by reference, The method involves inducing mutagenesis in a complementarity determining region (CDR) of an immunoglobulin light chain gene for the purpose of producing light chain gene libraries for use in combination with heavy chain genes and gene libraries to produce antibody libraries of diverse and novel immuno-specificities. The method comprises amplifying a CDR portion of an immunoglobulin light chain gene by polymerase chain reaction (PCR) using a PCR primer oligonucleotide. The resultant gene portions are inserted into phagemids for production of a phage display library, wherein the engineered light chains are displayed by the phages, for example for testing their binding specificity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using Papain produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. A scFv antibody fragment is an engineered antibody derivative that includes heavy- and light chain variable regions joined by a peptide linker. The minimal size of antibody molecules are those that still comprise the complete antigen binding site. ScFv antibody fragments are potentially more effective than unmodified IgG antibodies. The reduced size of 27-30 kDa permits them to penetrate tissues and solid tumors more readily. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)]. Optionally, there may be 1, 2 or 3 CDRs of different chains, but preferably there are 3 CDRs of 1 chain. The chain could be the heavy or the light chain.

Humanized forms of non-human (e.g., murine) antibodies, are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin, or fragments thereof may comprise the antibodies of this invention. Humanized antibodies are well known in the art. Methods for humanizing non-human antibodies are well known in the art, for example, as described in Winter and co-workers [Jones et al., Nature, 321:522-525 (1986); Riechmann et al., Nature 332:323-327 (1988); Verhoeyen et al., Science, 239:1534-1536 (1988)], U.S. Pat. No. 4,816,567, Hoogenboom and Winter, J. Mol. Biol., 227:381 (1991); Marks et al., J. Mol. Biol., 222:581 (1991), Cole et al., Monoclonal Antibodies and Cancer Therapy, Alan R. Liss, p. 77 (1985), Boerner et al., J. Immunol., 147(1):86-95 (1991), U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995), all of which are incorporated herein by reference.

Preferably, the antibody of this aspect of the present invention specifically binds at least one epitope of the polypeptide variants of the present invention. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

### Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Preferably used are antibodies which specifically interact with the polypeptides of the present invention and not with wild type proteins or other isoforms thereof, for example. Such antibodies are directed, for example, to the unique sequence portions of the polypeptide variants of the present invention, including but not limited to bridges, heads, tails and insertions described in greater detail below. Preferred embodiments of antibodies according to the present invention are described in greater detail with regard to the section entitled “Antibodies”.

Radio-immunoassay (RIA): In one version, this method involves precipitation of the desired substrate and in the methods detailed hereinbelow, with a specific antibody and radiolabelled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a calorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, calorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

### Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

### Display Libraries

According to still another aspect of the present invention there is provided a display library comprising a plurality of display vehicles (such as phages, viruses or bacteria) each displaying at least 6, at least 7, at least 8, at least 9, at least 10, 10-15, 12-17, 15-20, 15-30 or 20-50 consecutive amino acids derived from the polypeptide sequences of the present invention.

Methods of constructing such display libraries are well known in the art. Such methods are described in, for example, Young A C, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47):15430-5; Davies E L et al., “Selection of specific phage-display antibodies using libraries derived from chicken immunoglobulin genes” J Immunol Methods 1995 Oct. 12; 186(1):125-35; Jones C R T al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci USA 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

### Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

### Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities).

Surrogate endpoints were used first mainly in the cardiovascular area. For example, antihypertensive drugs have been approved based on their effectiveness in lowering blood pressure. Similarly, in the past, cholesterol-lowering agents have been approved based on their ability to decrease serum cholesterol, not on the direct evidence that they decrease mortality from atherosclerotic heart disease. The measurement of cholesterol levels is now an accepted surrogate marker of atherosclerosis. In addition, currently two commonly used surrogate markers in HIV studies are CD4+ T cell counts and quantitative plasma HIV RNA (viral load). In some embodiments of this invention, the polypeptide/polynucleotide expression pattern may serve as a surrogate marker for a particular disease, as will be appreciated by one skilled in the art.

### Monoclonal Antibody Therapy:

In some embodiments, monoclonal antibodies are useful for the identification of cancer cells. In some embodiments, monoclonal antibody therapy is a form of passive immunotherapy useful in cancer treatment. Such antibodies may comprise naked monoclonal antibodies or conjugated monoclonal antibodies—joined to a chemotherapy drug, radioactive particle, or a toxin (a substance that poisons cells). In some embodiments, the former is directly cytotoxic to the target (cancer) cell, or in another embodiment, stimulates or otherwise participates in an immune response ultimately resulting in the lysis of the target cell.

In some embodiments, the conjugated monoclonal antibodies are joined to drugs, toxins, or radioactive atoms. They are used as delivery vehicles to take those substances directly to the cancer cells. The MAb acts as a homing device, circulating in the body until it finds a cancer cell with a matching antigen. It delivers the toxic substance to where it is needed most, minimizing damage to normal cells in other parts of the body. Conjugated MAbs are also sometimes referred to as “tagged,” “labeled,” or “loaded” antibodies. MAbs with chemotherapy drugs attached are generally referred to as chemolabeled. MAbs with radioactive particles attached are referred to as radiolabeled, and this type of therapy is known as radioimmunotherapy (RIT). MAbs attached to toxins are called immunotoxins.

An illustrative, non-limiting example is provided herein of a method of treatment of a patient with an antibody to a variant as described herein, such that the variant is a target of the antibody. A patient with breast cancer is treated with a radiolabeled humanized antibody against an appropriate breast cancer target as described herein. The patient is optionally treated with a dosage of labeled antibody ranging from 10 to 30 mCi. Of course any type of therapeutic label may optionally be used.

The following sections relate to Candidate Marker Examples. It should be noted that Table numbering is restarted within each Example, which starts with the words “Description for Cluster”.

### Candidate Marker Examples Section

This Section relates to Examples of sequences according to the present invention, including illustrative methods of selection thereof with regard to cancer; other markers were selected as described below for the individual markers.

Description of the Methodology Undertaken to Uncover the Biomolecular Sequences of the Present Invention

Human ESTs and cDNAs were obtained from GenBank versions 136 (Jun. 15, 2003 ftp.ncbi.nih.gov/genbank/release.notes/gb136.release.notes); NCBI genome assembly of April 2003; RefSeq sequences from June 2003; Genbank version 139 (December 2003); Human Genome from NCBI (Build 34) (from October 2003); and RefSeq sequences from December 2003. With regard to GenBank sequences, the human EST sequences from the EST (GBEST) section and the human mRNA sequences from the primate (GBPRI) section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat. Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, J). Alu-containing exons are alternatively spliced. Genome Res 12, 1060-7 (2002); U.S. Pat. No. 6,625,545; and U.S. patent application Ser. No. 10/426,002, published as US20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

A brief explanation is provided with regard to the method of selecting the candidates. However, it should be noted that this explanation is provided for descriptive purposes only, and is not intended to be limiting in any way. The potential markers were identified by a computational process that was designed to find genes and/or their splice variants that are specifically expressed in cardiac tissue, as opposed to other types of tissues and also particularly as opposed to muscle tissue, by using databases of expressed sequences. Various parameters related to the information in the EST libraries, determined according to classification by library annotation, were used to assist in locating genes and/or splice variants thereof that are specifically and/or differentially expressed in heart tissues. The detailed description of the selection method and of these parameters is presented in Example 1 below.

### Cardiac Disease Markers

### Example 1

### Identification of Differentially Expressed Gene Products—Algorithm

In order to distinguish between differentially expressed gene products and constitutively expressed genes (i.e., house keeping genes), an algorithm based on an analysis of frequencies was configured. A specific algorithm for identification of transcripts specifically expressed in heart tissue is described hereinbelow.

EST Analysis

ESTs were taken from the following main sources: libraries contained in Genbank version 136 (Jun. 15, 2003 ftp.ncbi.nih.gov/genbank/release.notes/gb136.release.notes) and Genbank version 139 (December 2003); and from the LifeSeq library of Incyte Corporation (ESTs only; Wilmington, Del., USA). With regard to GenBank sequences, the human EST sequences from the EST (GBEST) section were used.

Library annotation—EST libraries were manually classified according to:
1. Tissue origin
2. Biological source—Examples of frequently used biological sources for construction of EST libraries include cancer cell-lines; normal tissues; cancer tissues; foetal tissues; and others such as normal cell lines and pools of normal cell-lines, cancer cell-lines and combinations thereof. A specific description of abbreviations used below with regard to these tissues/cell lines etc is given above.
3. Protocol of library construction—various methods are known in the art for library construction including normalized library construction; non-normalized library construction; subtracted libraries; ORESTES and others (described in the annotation available in Genbank). It will be appreciated that at times the protocol of library construction is not indicated in the information available about that library.

The following rules were followed:

EST libraries originating from identical biological samples were considered as a single library.

EST libraries which included above-average levels of contamination, such as DNA contamination for example, were eliminated. The presence of such contamination was determined as follows. For each library, the number of unspliced ESTs that are not fully contained within other spliced sequences was counted. If the percentage of such sequences (as compared to all other sequences) was at least 4 standard deviations above the average for all libraries being analyzed, this library was tagged as being contaminated and was eliminated from further consideration in the below analysis (see also Sorek, R. & Safer, H. M. A novel algorithm for computational identification of contaminated EST libraries. Nucleic Acids Res 31, 1067-74 (2003) for further details).

Clusters (genes) having at least five sequences including at least two sequences from the tissue of interest were analyzed. Splice variants were identified by using the LEADS software package as described above.

### Example 2

### Identification of Heart Tissue Specific Genes

For detection of heart tissue specific clusters, heart tissue libraries/sequences were compared to the total number of libraries/sequences in the cluster and in Genebank, and to the relevant numbers for muscle tissue libraries/sequences. Statistical tools were employed to identify clusters that were heart tissue specific, both as compared to all other tissues and also in comparison to muscle tissue.

The algorithm—for each tested tissue T and for each tested cluster the following were examined:

1. Each cluster includes at least 2 libraries from the tissue T. At least 3 clones (weighed—as described above) from tissue T in the cluster;

2. The following equation was then used to determine heart tissue-specific expression as compared to expression in all tissue types for a particular cluster:

$$\frac{t}{T} / \frac{n - t - m}{N - T - M}$$

in which n is the total number of ESTs available for a cluster, while N is the total number of ESTs available in all of the libraries considered in the analysis (effectively all ESTs in Genbank, except for those that were rejected as belonging to contaminated libraries). This ratio was preferably set to be at least about 8, although optionally the ratio could be set to be at least about 5.

3. The following equation was then used to determine heart tissue-specific expression vs. expression in skeletal muscle tissue for a particular cluster:

$$1 / \underline{T} / m / M$$

in which t represents the number of heart tissue-specific ESTs for the cluster, while T is the number of all heart tissue-specific ESTs in the analysis; m is the number of skeletal muscle tissue-specific ESTs for the cluster, while M is the number of all skeletal muscle tissue-specific ESTs in the analysis. This ratio was preferably set to be at least about 4, although optionally the ratio could be set to be at least about 2.

4. Fisher exact test P-values were computed for weighted clone counts to check that the counts are statistically significant according to the following function: F(t,T,n,N) which is the probability of a cluster actually being overexpressed in heart tissue, as compared to its overall level of expression. The P-value was preferably set to be less than about 1e-5, although optionally it could be set to be less than about 1e-3.

Selecting Candidates with Regard to Cancer

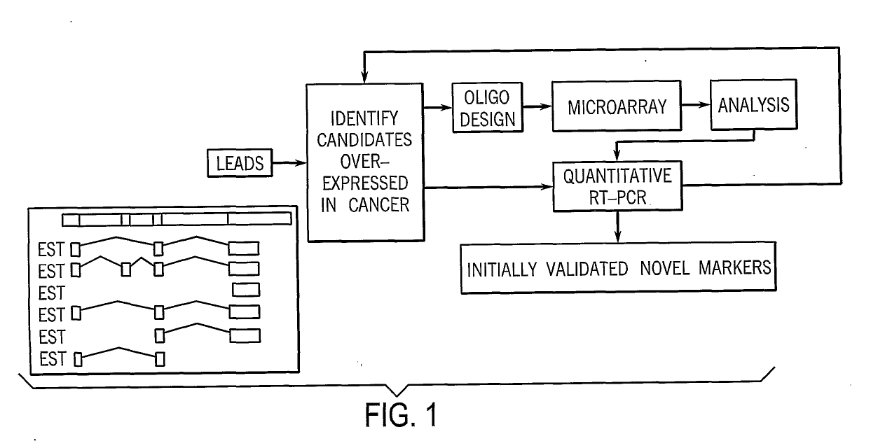

A brief explanation is provided with regard to a non-limiting method of selecting the candidates for cancer diagnostics. However, it should noted that this explanation is provided for descriptive purposes only, and is not intended to be limiting in any way. The potential markers were identified by a computational process that was designed to find genes and/or their splice variants that are over-expressed in tumor tissues, by using databases of expressed sequences. Various parameters related to the information in the EST libraries, determined according to a manual classification process, were used to assist in locating genes and/or splice variants thereof that are over-expressed in cancerous tissues. The detailed description of the selection method is presented in Example 1 below. The cancer biomarkers selection engine and the following wet validation stages are schematically summarized in FIG. 1.

### Part II—Cancer Markers

### Example 3

### Identification of Differentially Expressed Gene Products—Algorithm

In order to distinguish between differentially expressed gene products and constitutively expressed genes (i.e., house keeping genes) an algorithm based on an analysis of frequencies was configured. A specific algorithm for identification of transcripts over expressed in cancer is described hereinbelow.

Dry Analysis

Library annotation—EST libraries are manually classified according to:
(i) Tissue origin
(ii) Biological source—Examples of frequently used biological sources for construction of EST libraries include cancer cell-lines; normal tissues; cancer tissues; fetal tissues; and others such as normal cell lines and pools of normal cell-lines, cancer cell-lines and combinations thereof. A specific description of abbreviations used below with regard to these tissues/cell lines etc is given above.
(iii) Protocol of library construction—various methods are known in the art for library construction including normalized library construction; non-normalized library construction; subtracted libraries; ORESTES and others. It will be appreciated that at times the protocol of library construction is not indicated.

The following rules are followed:

EST libraries originating from identical biological samples are considered as a single library.

EST libraries which include above-average levels of DNA contamination are eliminated.

Dry computation—development of engines which are capable of identifying genes and splice variants that are temporally and spacially expressed.

Clusters (genes) having at least five sequences including at least two sequences from the tissue of interest are analyzed.

### Example 4

### Identification of Genes Over Expressed in Cancer

Two Different Scoring Algorithms were Developed.

Libraries score—candidate sequences which are supported by a number of cancer libraries, are more likely to serve as specific and effective diagnostic markers.

The basic algorithm—for each cluster the number of cancer and normal libraries contributing sequences to the cluster was counted. Fisher exact test was used to check if cancer libraries are significantly over-represented in the cluster as compared to the total number of cancer and normal libraries.

Library counting: Small libraries (e.g., less than 1000 sequences) were excluded from consideration unless they participate in the cluster. For this reason, the total number of libraries is actually adjusted for each cluster.

Clones no. score—Generally, when the number of ESTs is much higher in the cancer libraries relative to the normal libraries it might indicate actual over-expression.

The algorithm—

Clone counting: For counting EST clones each library protocol class was given a weight based on our belief of how much the protocol reflects actual expression levels:

(i) non-normalized: 1

(ii) normalized: 0.2

(iii) all other classes: 0.1

Clones number score—The total weighted number of EST clones from cancer libraries was compared to the EST clones from normal libraries. To avoid cases where one library contributes to the majority of the score, the contribution of the library that gives most clones for a given cluster was limited to 2 clones.

The score was computed as

$$\frac{c + 1}{C} / \frac{n + 1}{N}$$

where:

c—weighted number of “cancer” clones in the cluster.

C—weighted number of clones in all “cancer” libraries.

n—weighted number of “normal” clones in the cluster.

N—weighted number of clones in all “normal” libraries.

Clones number score significance—Fisher exact test was used to check if EST clones from cancer libraries are significantly over-represented in the cluster as compared to the total number of EST clones from cancer and normal libraries.

Two search approaches were used to find either general cancer-specific candidates or tumor specific candidates.
Libraries/sequences originating from tumor tissues are counted as well as libraries originating from cancer cell-lines (“normal” cell-lines were ignored).
Only libraries/sequences originating from tumor tissues are counted

### Example 5

### Identification of Tissue Specific Genes

For detection of tissue specific clusters, tissue libraries/sequences were compared to the total number of libraries/sequences in cluster. Similar statistical tools to those described in above were employed to identify tissue specific genes. Tissue abbreviations are the same as for cancerous tissues, but are indicated with the header “normal tissue”.

The algorithm—for each tested tissue T and for each tested cluster the following were examined:

1. Each cluster includes at least 2 libraries from the tissue T. At least 3 clones (weighed—as described above) from tissue T in the cluster; and

2. Clones from the tissue T are at least 40% from all the clones participating in the tested cluster

Fisher exact test P-values were computed both for library and weighted clone counts to check that the counts are statistically significant.

### Example 6

### Identification of Cancer Specific Splice Variants of Genes Over Expressed in Cancer

A search for EST supported (no mRNA) regions for genes of:

(i) known cancer markers

(ii) Genes shown to be over-expressed in cancer in published micro-array experiments.

Reliable EST supported-regions were defined as supported by minimum of one of the following:

(i) 3 spliced ESTs; or

(ii) 2 spliced ESTs from 2 libraries;

(iii) 10 unspliced ESTs from 2 libraries, or

(iv) 3 libraries.

### Example 7

### Oligonucleotide-Based Micro-Array Experiment Protocol

### Microarray Fabrication

Microarrays (chips) were printed by pin deposition using the MicroGrid II MGII 600 robot from BioRobotics Limited (Cambridge, UK). 50-mer oligonucleotides target sequences were designed by Compugen Ltd (Tel-Aviv, Ill.) as described by A. Shoshan et al, “Optical technologies and informatics”, Proceedings of SPIE. Vol 4266, pp. 86-95 (2001). The designed oligonucleotides were synthesized and purified by desalting with the Sigma-Genosys system (The Woodlands, Tex., US) and all of the oligonucleotides were joined to a C6 amino-modified linker at the 5′ end, or being attached directly to CodeLink slides (Cat #25-6700-01. Amersham Bioscience, Piscataway, N.J., US). The 50-mer oligonucleotides, forming the target sequences, were first suspended in Ultra-pure DDW (Cat # 01-866-1A Kibbutz Beit-Haemek, Israel) to a concentration of 50 μM. Before printing the slides, the oligonucleotides were resuspended in 300 mM sodium phosphate (pH 8.5) to final concentration of 150 mM and printed at 35-40% relative humidity at 21° C.

Each slide contained a total of 9792 features in 32 subarrays. Of these features, 4224 features were sequences of interest according to the present invention and negative controls that were printed in duplicate. An additional 288 features (96 target sequences printed in triplicate) contained housekeeping genes from Human Evaluation Library2, Compugen Ltd, Israel. Another 384 features are E. coli spikes 1-6, which are oligos to E. Coli genes which are commercially available in the Array Control product (Array control—sense oligo spots, Ambion Inc. Austin, Tex. Cat # 1781, Lot #112K06).

### Post-Coupling Processing of Printed Slides

After the spotting of the oligonucleotides to the glass (CodeLink) slides, the slides were incubated for 24 hours in a sealed saturated NaCl humidification chamber (relative humidity 70-75%).

Slides were treated for blocking of the residual reactive groups by incubating them in blocking solution at 50° C. for 15 minutes (10 ml/slide of buffer containing 0.1M Tris, 50 mM ethanolamine, 0.1% SDS). The slides were then rinsed twice with Ultra-pure DDW (double distilled water). The slides were then washed with wash solution (10 ml/slide. 4×SSC, 0.1% SDS) at 50° C. for 30 minutes on the shaker. The slides were then rinsed twice with Ultra-pure DDW, followed by drying by centrifugation for 3 minutes at 800 rpm.

Next, in order to assist in automatic operation of the hybridization protocol, the slides were treated with Ventana Discovery hybridization station barcode adhesives. The printed slides were loaded on a Bio-Optica (Milan, Italy) hematology staining device and were incubated for 10 minutes in 50 ml of 3-Aminopropyl Triethoxysilane (Sigma A3648 lot #122K589). Excess fluid was dried and slides were then incubated for three hours in 20 mm/Hg in a dark vacuum desiccator (Pelco 2251, Ted Pella, Inc. Redding Calif.).

The following protocol was then followed with the Genisphere 900-RP (random primer), with mini elute columns on the Ventana Discovery HybStation™, to perform the microarray experiments. Briefly, the protocol was performed as described with regard to the instructions and information provided with the device itself. The protocol included cDNA synthesis and labeling. cDNA concentration was measured with the TBS-380 (Turner Biosystems. Sunnyvale, Calif.) PicoFlour, which is used with the OliGreen ssDNA Quantitation reagent and kit.

Hybridization was performed with the Ventana Hybridization device, according to the provided protocols (Discovery Hybridization Station Tuscon Ariz.).

The slides were then scanned with GenePix 4000B dual laser scanner from Axon Instruments Inc, and analyzed by GenePix Pro 5.0 software.

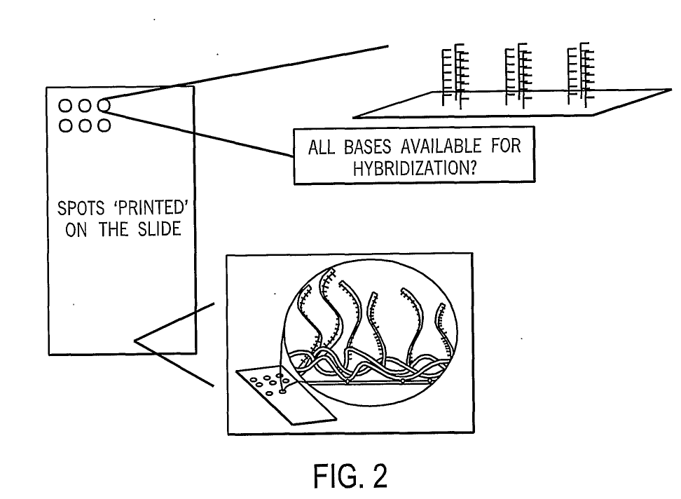

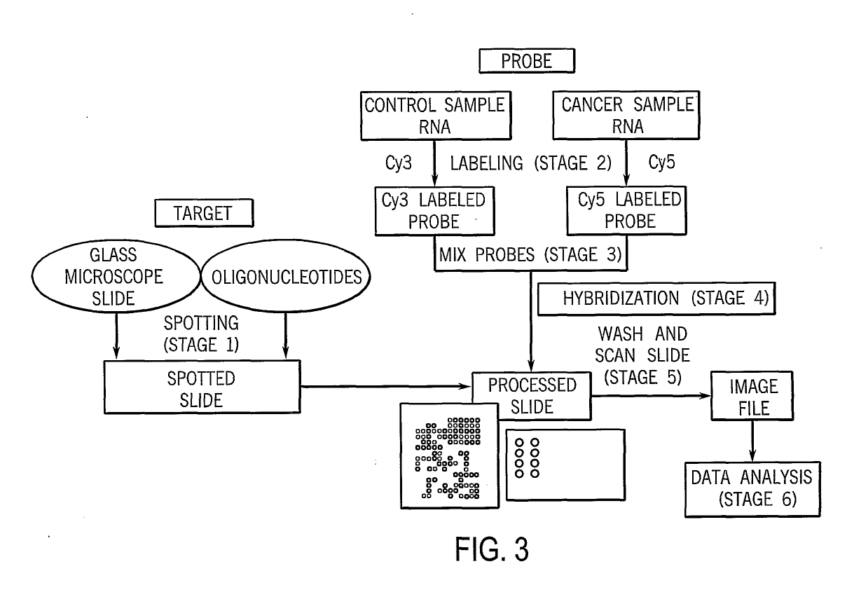

Schematic summary of the oligonucleotide based microarray fabrication and the experimental flow is presented in FIGS. 2 and 3.

Briefly, as shown in FIG. 2, DNA oligonucleotides at 25 uM were deposited (printed) onto Amersham ‘CodeLink’ glass slides generating a well defined ‘spot’. These slides are covered with a long-chain, hydrophilic polymer chemistry that creates an active 3-D surface that covalently binds the DNA oligonucleotides 5′-end via the C6-amine modification. This binding ensures that the full length of the DNA oligonucleotides is available for hybridization to the cDNA and also allows lower background, high sensitivity and reproducibility.

FIG. 3 shows a schematic method for performing the microarray experiments. It should be noted that stages on the left-hand or right-hand side may optionally be performed in any order, including in parallel, until stage 4 (hybridization). Briefly, on the left-hand side, the target oligonucleotides are being spotted on a glass microscope slide (although optionally other materials could be used) to form a spotted slide (stage 1). On the right hand side, control sample RNA and cancer sample RNA are Cy3 and Cy5 labeled, respectively (stage 2), to form labeled probes. It should be noted that the control and cancer samples come from corresponding tissues (for example, normal prostate tissue and cancerous prostate tissue). Furthermore, the tissue from which the RNA was taken is indicated below in the specific examples of data for particular clusters, with regard to overexpression of an oligonucleotide from a “chip” (microarray), as for example “prostate” for chips in which prostate cancerous tissue and normal tissue were tested as described above. In stage 3, the probes are mixed. In stage 4, hybridization is performed to form a processed slide. In stage 5, the slide is washed and scanned to form an image file, followed by data analysis in stage 6.

### Example 8

### Diseases and Conditions that May be Diagnosed with One or More Variants According to the Present Invention

### Cardiovascular and Cerebrovascular Conditions

Various examples are listed below for conditions that affect the vascular system, including various cardiovascular and cerebrovascular conditions, for which one or more variants according to the present invention may have a diagnostic utility. Based on these diseases mechanisms and the correlation between the known proteins and the cardiovascular and cerebrovascular conditions, such correlation was predicted also for one or more variants of cluster S56200 according to the present invention, as described below. Each variant marker of the present invention described herein as potential marker for cardiovascular conditions, might optionally be used alone or in combination with one or more other variant markers described herein, and or in combination with known markers for cardiovascular conditions, including but not limited to Heart-type fatty acid binding protein (H-FABP), Angiotensin, C-reactive protein (CRP), myeloperoxidase (MPO), and/or in combination with the known protein(s) for the variant marker as described herein. Each variant marker of the present invention described herein as potential marker for cerebrovascular conditions, might optionally be used alone or in combination with one or more other variant markers described herein, and or in combination with known markers for cerebrovascular conditions, including but not limited to CRP, S100b, BNGF, CD40, MCP1, N-Acetyl-Aspartate (NAA), N-methyl-d-aspartate (NMDA) receptor antibodies (NR2Ab), and/or in combination with the known protein(s) for the variant marker as described herein.

### Myocardial Infarction

Cluster S56200 variants are potential markers for myocardial infarction. Other conditions that may be diagnosed by these markers or variants of them include but are not limited to the presence, risk and/or extent of the following:
1. Myocarditis—in myocarditis cardiac muscle cells can go through cell lysis and leakage with the release of intracellular content to the extracellular space and blood, a similar process as happens in myocardial infarction (see also extended description below).
2. Angina—stable or unstable, as the reduction of oxygen delivery to part of the heart often leads to local ischemic conditions that facilitate leakage of intracellular content.
3. Traumatic injury to myocardial tissue—blunt or penetrating, may also result in myocardial cell leakage.
4. Opening an occluded coronary artery following thrombolytic therapy—If such treatment is successful, proteins and other products of the local tissue are washed into the blood and can be detected there.
5. Cardiomyopathy—which is characterized by slow degeneration of the heart muscle (see also extended description below).
6. Myocardial injury after rejection of heart transplant.
7. Congestive heart failure where heart myocytes slowly degenerate (as had been shown for Troponin-I; see also extended description below).
8. Future cardiovascular disease (as a risk factor).
9. Conditions which have similar clinical symptoms as myocardial infarction and where the differential diagnosis between them and myocardial infarction is of clinical importance including but not limited to:
   a. Clinical symptoms resulting from lung related tissue (e.g. Pleuritis, pulmonary embolism)
   b. Musculoskeletal origin of pain
   c. Clinical symptoms resulting from heart related tissue which are not due to myocardial infarction, e.g. acute pericarditis
   d. Upper abdominal pain from abdominal organs including but nor limited to esophagitis, gastro-esophageal reflux, gastritis, gastric ulcer, duodenitis, duodenal ulcer, enteritis, gastroenteritis, cholecystitis, cholelithiasis, cholangiolithiasis, pancreatitis, splenic infarction, splenic trauma, Aortic dissection.

One or more of these markers (variants according to the present invention) may optionally be used a tool to decide on treatment options e.g. anti platelet inhibitors (as has been shown for Troponin-I); as a tool in the assessment of pericardial effusion; and/or as a tool in the assessment of endocarditis and/or rheumatic fever, where progressive damage to the heart muscle may occur.

### Cardiomyopathy and Myocarditis

Cardiomyopathy may be treated with the polynucleotides/polypeptides and/or methods of this invention. Cardiomyopathy is a general diagnostic term designating primary myocardial disease which may progress to heart failure. The disease comprises inflammatory cardiomyopathies, cardiomyopathies resulting from a metabolic disorder such as a nutritional deficiency or by altered endocrine function, exposure to toxic substances, for example from alcohol or exposure to cobalt or lead, infiltration and deposition of abnormal. In some embodiments, the marker(s) for diagnosis of cardiomyopathy and myocarditis, and related conditions as described herein, may optionally be selected from the group consisting of cluster S56200 variants

### Congestive Heart Failure (CHF)

Cluster S56200 variants are potential markers for, and may be used to treat, etc., CHF.

The invention provides a means for the identification/prognostication, etc., of a number of conditions including the assessment of the presence, risk and/or extent of the following:
1. A risk factor for sudden cardiac death, from arrhythmia or any other heart related reason.
2. Rejection of a transplanted heart.
3. Conditions that lead to heart failure including but not limited to myocardial infarction, angina, arrhythmias, valvular diseases, atrial and/or ventricular septal defects.
4. Conditions that cause atrial and or ventricular wall volume overload. Wall stretch results in enhanced secretion of cardiac extracellular regulators. Such conditions include but are not limited to systemic arterial hypertension, pulmonary hypertension and pulmonary embolism.
5. Conditions which have similar clinical symptoms as heart failure and as states that cause atrial and or ventricular pressure-overload, where the differential diagnosis between these conditions to the latter is of clinical importance including but not limited to breathing difficulty and/or hypoxia due to pulmonary disease, anemia or anxiety.

### Acute and Chronic Inflammation and Risk Factors for CVS Diseases

Cluster S56200 variants are potential markers for inflammation, including a spectrum of diseases where an inflammatory process plays a substantial role. In addition CRP levels and in particular baseline levels serve as a risk factor for various diseases, particularly cardiovascular diseases where inflammation is thought to participate in the pathogenesis. Conditions that may be diagnosed by these markers or variants of them include but are not limited to the presence, risk and/or extent of the following:
1. Conditions that entail an inflammatory process that involves blood vessels including but not limited to hypercholesterolemia, diabetes, atherosclerosis, inflammation that involves blood vessels—whether acute or chronic including but not limited to the coronary arteries and blood vessels of the brain, myocardial infarction, cerebral stroke, peripheral vascular disease, vasculitis, polyarteritis nodosa, ANCA associated small vessel vasculitis, Churg-Strauss syndrome, Henoch-Schonlein purpura, scleroderma, thromboangiitis obliterans, temporal arteritis, Takayasu's arteritis, hypersensitivity vasculitis, Kawasaki disease, Behçet syndrome, and their complications including but not limited to coronary disease, angina pectoris, deep vein thrombosis, renal disease, diabetic nephropathy, lupus nephritis, renal artery thrombosis, renal artery stenosis, atheroembolic disease of the renal arteries, renal vein thrombosis, hemolytic uremic syndrome, thrombotic thrombocytopenic purpura, arteriolar nephrosclerosis, preeclampsia, eclampsia, albuminuria, microalbuminuria, glomerulonephritis, renal failure, hypertension, uremia, cerebrovascular disease, peripheral vascular disease, intermittent claudication, abdominal angina.
2. Rheumatic/autoimmune diseases that involve systemic immune reaction including but not limited to rheumatoid arthritis, scleroderma, mixed connective tissue disease, Sjogren syndrome, ankylosing spondylitis, spondyloarthropathy, psoriasis, psoriatic arthritis, myositis and systemic lupus erythematosus.
3. Acute and/or chronic infective processes that involve systemic immune reaction including but not limited to pneumonia, bacteremia, sepsis, pyelonephritis, cellulitis, osteomyelitis, meningitis and viral hepatitis.
4. Malignant and idiopathic processes that involve systemic immune reaction and/or proliferation of immune cells including but not limited to granulomatous disorders, Wegener's granulomatosis, lymphomatoid granulomatosis/polymorphic reticulosis, idiopathic midline granuloma, multiple myeloma, Waldenstrom's macroglobulinemia, Castleman's disease, amyloidosis, lymphoma, histiocytosis, renal cell carcinoma and paraneoplastic syndromes.
5. Conditions where CRP was shown to have a positive correlation with the presence of the condition including but not limited to weight loss, anorexia-cachexia syndrome, extent of disease, recurrence in advanced cancer, diabetes (types 1 & 2), obesity, hypertension, preterm delivery.
6. Conditions which have similar symptoms, signs and complications as the conditions above and where the differential diagnosis between them and the conditions above is of clinical importance including but not limited to:
   a. Other (non vascular) causes of heart disease, renal disease and cerebral disease.
   b. Other (non rheumatic) causes of arthropathy and musculoskeletal pain.
   c. Other causes of non-specific symptoms and signs such as fever of unknown origin, loss of appetite, weight loss, nonspecific pains, breathing difficulties and anxiety.

### Stroke

Stroke is a manifestation of vascular injury to the brain which is commonly secondary to atherosclerosis or hypertension, and is the third leading cause of death (and the second most common cause of neurologic disability) in the United States. Embodiments of marker(s) for diagnosis of stroke and related conditions as described herein may optionally be selected from the group consisting of cluster S56200 variants or markers related thereto.

Specific markers of neural tissue injury are found in the blood or in blood components such as serum and plasma, as well as the CSF of a patient experiencing stroke or TIAs. Furthermore, clearance of the obstructing object in ischemic stroke can cause injury from oxidative insult during reperfusion, and patients with ischemic stroke can sometimes experience hemorrhagic transformation as a result of reperfusion or thrombolytic therapy.

Fibrinolysis is the process of proteolytic clot dissolution. In a manner analogous to coagulation, fibrinolysis is mediated by serine proteinases that are activated from their zymogen form. The serine proteinase plasmin is responsible for the degradation of fibrin into smaller degradation products that are liberated from the clot, resulting in clot dissolution. Fibrinolysis is activated soon after coagulation in order to regulate clot formation. Endogenous serine proteinase inhibitors also function as regulators of fibrinolysis.

The presence of a coagulation or fibrinolysis marker in cerebrospinal fluid would indicate that activation of coagulation or fibrinolysis, depending upon the marker used, coupled with increased permeability of the blood-brain barrier has occurred. In this regard, more definitive conclusions regarding the presence of coagulation or fibrinolysis markers associated with acute stroke may be obtained using cerebrospinal fluid.

Stroke can be categorized into two broad types, “ischemic stroke” and “hemorrhagic stroke.” Additionally, a patient may experience transient ischemic attacks, which are in turn a high risk factor for the future development of a more severe episode.

Ischemic stroke encompasses thrombotic, embolic, lacunar and hypoperfusion types of strokes. Thrombi are occlusions of arteries created in situ within the brain, while emboli are occlusions caused by material from a distant source, such as the heart and major vessels, often dislodged due to myocardial infarct or atrial fibrillation. Less frequently, thrombi may also result from vascular inflammation due to disorders such as meningitis. Thrombi or emboli can result from atherosclerosis or other disorders, for example, arteritis, and lead to physical obstruction of arterial blood supply to the brain. Lacunar stroke refers to an infarct within non-cortical regions of the brain. Hypoperfusion embodies diffuse injury caused by non-localized cerebral ischemia, typically caused by myocardial infarction and arrhythmia.

The onset of ischemic stroke is often abrupt, and can become an “evolving stroke” manifested by neurologic deficits that worsen over a 24-48 hour period. In evolving stroke, “stroke-associated symptom(s)” commonly include unilateral neurologic dysfunction which extends progressively, without producing headache or fever. Evolving stroke may also become a “completed stroke,” in which symptoms develop rapidly and are maximal within a few minutes.

Hemorrhagic stroke is caused by intracerebral or subarachnoid hemorrhage, i.e., bleeding into brain tissue, following blood vessel rupture within the brain. Intracerebral and subarachnoid hemorrhage are subsets of a broader category of hemorrhage referred to as intracranial hemorrhage. Intracerebral hemorrhage is typically due to chronic hypertension, and a resulting rupture of an arteriosclerotic vessel. Stroke-associated symptom(s) of intracerebral hemorrhage are abrupt, with the onset of headache and steadily increasing neurological deficits. Nausea, vomiting, delirium, seizures and loss of consciousness are additional common stroke-associated symptoms.

In contrast, most subarachnoid hemorrhage is caused by head trauma or aneurysm rupture which is accompanied by high pressure blood release which also causes direct cellular trauma. Prior to rupture, aneurysms may be asymptomatic, or occasionally associated with tension or migraine headaches. However, headache typically becomes acute and severe upon rupture, and may be accompanied by varying degrees of neurological deficit, vomiting, dizziness, and altered pulse and respiratory rates.

Transient ischemic attacks (TIAs) have a sudden onset and brief duration, typically 2-30 minutes. Most TIAs are due to emboli from atherosclerotic plaques, often originating in the arteries of the neck, and can result from brief interruptions of blood flow. The symptoms of TIAs are identical to those of stroke, but are only transient. Concomitant with underlying risk factors, patients experiencing TIAs are at a markedly increased risk for stroke.

Current diagnostic methods for stroke include costly and time-consuming procedures such as noncontrast computed tomography (CT) scan, electrocardiogram, magnetic resonance imaging (MRI), and angiography. Determining the immediate cause of stroke and differentiating ischemic from hemorrhagic stroke is difficult. CT scans can detect parenchymal bleeding greater than 1 cm and 95% of all subarachnoid hemorrhages. CT scan often cannot detect ischemic strokes until 6 hours from onset, depending on the infarct size. MRI may be more effective than CT scan in early detection of ischemic stroke, but it is less accurate at differentiating ischemic from hemorrhagic stroke, and is not widely available. An electrocardiogram (ECG) can be used in certain circumstances to identify a cardiac cause of stroke. Angiography is a definitive test to identify stenosis or occlusion of large and small cranial blood vessels, and can locate the cause of subarachnoid hemorrhages, define aneurysms, and detect cerebral vasospasm. It is, however, an invasive procedure that is also limited by cost and availability. Coagulation studies can also be used to rule out a coagulation disorder (coagulopathy) as a cause of hemorrhagic stroke.

Immediate diagnosis and care of a patient experiencing stroke can be critical. For example, tissue plasminogen activator (TPA) given within three hours of symptom onset in ischemic stroke is beneficial for selected acute stroke patients. Alternatively, patients may benefit from anticoagulants (e.g., heparin) if they are not candidates for TPA therapy. In contrast, thrombolytics and anticoagulants are strongly contraindicated in hemorrhagic strokes. Thus, early differentiation of ischemic events from hemorrhagic events is imperative. Moreover, delays in the confirmation of stroke diagnosis and the identification of stroke type limit the number of patients that may benefit from early intervention therapy. Finally, there are currently no diagnostic methods that can identify a TIA, or predict delayed neurological deficits which are often detected at a time after onset concurrent with the presentation of symptoms.

Accordingly, there is a present need in the art for a rapid, sensitive and specific diagnostic assay for stroke and TIA that can also differentiate the stroke type and identify those individuals at risk for delayed neurological deficits. Such a diagnostic assay would greatly increase the number of patients that can receive beneficial stroke treatment and therapy, and reduce the costs associated with incorrect stroke diagnosis.

The present invention relates to the identification and use of diagnostic markers for stroke and neural tissue injury. The methods and compositions described herein can meet the need in the art for rapid, sensitive and specific diagnostic assay to be used in the diagnosis and differentiation of various forms of stroke and TIAs. Moreover, the methods and compositions of the present invention can also be used to facilitate the treatment of stroke patients and the development of additional diagnostic and/or prognostic indicators.

In various aspects, the invention relates to materials and procedures for identifying markers that are associated with the diagnosis, prognosis, or differentiation of stroke and/or TIA in a patient; to using such markers in diagnosing and treating a patient and/or to monitor the course of a treatment regimen; to using such markers to identify subjects at risk for one or more adverse outcomes related to stroke and/or TIA; and for screening compounds and pharmaceutical compositions that might provide a benefit in treating or preventing such conditions.

In a first aspect, the invention discloses methods for determining a diagnosis or prognosis related to stroke, or for differentiating between types of strokes and/or TIA. These methods comprise analyzing a test sample obtained from a subject for the presence or amount of one or more markers for neural tissue injury. These methods can comprise identifying one or more markers, the presence or amount of which is associated with the diagnosis, prognosis, or differentiation of stroke and/or TIA. Once such marker(s) are identified, the level of such marker(s) in a sample obtained from a subject of interest can be measured. In certain embodiments, these markers can be compared to a level that is associated with the diagnosis, prognosis, or differentiation of stroke and/or TIA. By correlating the subject's marker level(s) to the diagnostic marker level(s), the presence or absence of stroke, the probability of future adverse outcomes, etc., in a patient may be rapidly and accurately determined.

In a related aspect, the invention discloses methods for determining the presence or absence of a disease in a subject that is exhibiting a perceptible change in one or more physical characteristics (that is, one or more “symptoms”) that are indicative of a plurality of possible etiologies underlying the observed symptom(s), one of which is stroke. These methods comprise analyzing a test sample obtained from the subject for the presence or amount of one or more markers selected to rule in or out stroke, or one or more types of stroke, as a possible etiology of the observed symptom(s). Etiologies other than stroke that are within the differential diagnosis of the symptom(s) observed are referred to herein as “stroke mimics”, and marker(s) able to differentiate one or more types of stroke from stroke mimics are referred to herein as “stroke differential diagnostic markers”. The presence or amount of such marker(s) in a sample obtained from the subject can be used to rule in or rule out one or more of the following: stroke, thrombotic stroke, embolic stroke, lacunar stroke, hypoperfusion, intracerebral hemorrhage, and subarachnoid hemorrhage, thereby either providing a diagnosis (rule-in) and/or excluding a diagnosis (rule-out).

Obtaining information on the true time of onset can be critical, as early treatments have been reported to be critical for proper treatment. Obtaining this time-of-onset information may be difficult, and is often based upon interviews with companions of the stroke victim. Thus, in various embodiments, markers and marker panels are selected to distinguish the approximate time since stroke onset. For purposes of the present invention, the term “acute stroke” refers to a stroke that has occurred within the prior 12 hours, more preferably within the prior 6 hours, and most preferably within the prior 3 hours; while the term “non-acute stroke” refers to a stroke that has occurred more than 12 hours ago, preferably between 12 and 48 hours ago, and most preferably between 12 and 24 hours ago. Embodiments of markers for differentiating between acute and non-acute strokes, referred to herein as stroke “time of onset markers” are described hereinafter.

For markers appearing in the patent which are already linked to stroke, either ischemic or hemorrhagic, variants could also help to diagnose, directly or by elimination of other conditions including but not limited to:
1. Transient ischemic attack
2. Brain trauma, in case it is unclear whether accompanied by stroke or not
3. Migraine
4. Bleeding in any part of the brain or inside the skull that cause or didn't cause damage to brain tissue
5. Tumor
   In addition, such markers may help determine:
1. The time of stroke
2. The type of stroke
3. The extent of tissue damage as a result of the stroke
4. Response to immediate treatments that are meant to alleviate the extent of stroke and brain damage, when available.

With regard to stroke, according to embodiments of the present invention, the panel may optionally and preferably provide diagnosis of stroke and indication if an ischemic stroke has occurred; diagnosis of stroke and indication if a hemorrhagic stroke has occurred; diagnosis of stroke, indication if an ischemic stroke has occurred, and indication if a hemorrhagic stroke has occurred; diagnosis of stroke and prognosis of a subsequent cerebral vasospasm; and diagnosis of stroke, indication if a hemorrhagic stroke has occurred, and prognosis of a subsequent cerebral vasospasm.

According to other optional embodiments of the present invention, there are provided methods of identifying a patient at risk for cerebral vasospasm. Such methods preferably comprise comparing an amount of one or more marker(s) predictive of a subsequent cerebral vasospasm in a test sample from a patient diagnosed with a subarachnoid hemorrhage. Such markers may be one or more markers related to blood pressure regulation, markers related to inflammation, markers related to apoptosis, and/or specific markers of neural tissue injury. As discussed herein, such marker may be used in panels comprising 2, 3, 4, 5, 6, 7, 8, 9, 10, 15, 20, or more or individual markers. Embodiments of marker(s) may be selected from the group consisting of cluster S56200 variants or markers related thereto. The levels of one or more markers may be compared to a predictive level of said marker(s), wherein said patient is identified as being at risk for cerebral vasospasm by a level of said marker(s) equal to or greater than said predictive level. In the alternative, a panel response value for a plurality of such markers may be determined, optionally considering a change in the level of one or more such markers as an additional independent marker.

According to yet other embodiments of the present invention, there are provided methods of differentiating ischemic stroke from hemorrhagic stroke using such marker panels.

### Cancerous Conditions

Various non-limiting examples are given below of cancerous conditions for which one or more variants according to the present invention may have a diagnostic, or therapeutic utility.

### Ovarian Cancer

Ovarian cancer causes more deaths than any other cancer of the female reproductive system, however, only 25% of ovarian cancers are detected in stage I. No single marker has been shown to be sufficiently sensitive or specific to contribute to the diagnosis of ovarian cancer.

In one embodiment, the markers of this invention are utilized alone, or in combination with other markers, for the diagnosis, treatment or assessment of prognosis of ovarian cancer. Such other markers may comprise CA-125 or mucin 16, CA-50, CA 54-61, CA-195 and CA 19-9, STN and TAG-72, kallikreins, cathepsin L, urine gonadotropin, inhibins, cytokeratins, such as TPA and TPS, members of the Transforming Growth Factors (TGF) beta superfamily, Epidermal Growth Factor, p53 and HER-2 or any combination thereof.

Immunohistochemistry may be used to assess the origin of the tumor and staging as part of the methods of this invention, and as protected uses for the polypeptides of this invention.

In some embodiments, this invention provides polypeptides/polynucleotides which serves as markers for ovarian cancer. In some embodiments, the marker is any polypeptide/polynucleotide as described herein. In some embodiments, the marker is HSCP2, or variants as described herein or markers related thereto. Each variant marker of the present invention described herein may be used alone or in combination with one or more other variant ovarian cancer described herein, and/or in combination with known markers for ovarian cancer, as described herein. Diagnosis of ovarian cancer and/or of other conditions that may be diagnosed by these markers or variants of them, include but are not limited to the presence, risk and/or extent of the following:
1. The identification of a metastasis of unknown origin which originated from a primary ovarian cancer.
2. As a marker to distinguish between different types of ovarian cancer, therefore potentially affect treatment choice (e.g. discrimination between epithelial tumors and germ cell tumors).
3. As a tool in the assessment of abdominal mass and in particular in the differential diagnosis between a benign and malignant ovarian cysts.
4. As a tool for the assessment of infertility.
5. Other conditions that may elevate serum levels of ovary related markers. These include but are not limited to: cancers of the endometrium, cervix, fallopian tubes, pancreas, breast, lung and colon; nonmalignant conditions such as pregnancy, endometriosis, pelvic inflammatory disease and uterine fibroids.
6. Conditions which have similar symptoms, signs and complications as ovarian cancer and where the differential diagnosis between them and ovarian cancer is of clinical importance including but not limited to:
   a. Non-malignant causes of pelvic mass. Including, but not limited to: benign (functional) ovarian cyst, uterine fibroids, endometriosis, benign ovarian neoplasms and inflammatory bowel lesions
   b. Any condition suggestive of a malignant tumor including but not limited to anorexia, cachexia, weight loss, fever, hypercalcemia, skeletal or abdominal pain, paraneoplastic syndrome.
   c. Ascites.
7. Prediction of patient's drug response
8. As surrogate markers for clinical outcome of a treated cancer.
9. Screening for early detection of ovarian cancer.

### Lung Cancer

Lung cancer is the primary cause of cancer death among both men and women in the U. S. In one embodiment, the polypeptides and/or polynucleotides of this invention are utilized alone, or in combination with other markers, for the diagnosis, treatment or assessment of prognosis of lung cancer. In one embodiment, the term “lung cancer” is to be understood as encompassing small cell or non-small cell lung cancers, including adenocarcinomas, bronchoalveolar-alveolar, squamous cell and large cell carcinomas.

In some embodiments, the polypeptides/polynucleotides of this invention are utilized in conjunction with other screening procedures, as well as the use of other markers, for the diagnosis, or assessment of prognosis of lung cancer in a subject. In some embodiments, such screening procedures may comprise the use of chest x-rays, analysis of the type of cells contained in sputum, fiberoptic examination of the bronchial passages, or any combination thereof. Such evaluation in turn may impact the type of treatment regimen pursued, which in turn may reflect the type and stage of the cancer, and include surgery, radiation therapy and/or chemotherapy.

Current radiotherapeutic agents, chemotherapeutic agents and biological toxins are potent cytotoxins, yet do not discriminate between normal and malignant cells, producing adverse effects and dose-limiting toxicities. In some embodiments of this invention, the polypeptides/polynucleotides provide a means for more specific targeting to neoplastic versus normal cells.

In some embodiments, the polypeptides for use in the diagnosis, treatment and/or assessment of progression of lung cancer may comprise: cluster HSCP2 variants, cluster S56200 variants or homologous thereof, or polynucleotides encoding the same. In some embodiments, these polypeptides/polynucleotides may be used alone or in combination with one or more other appropriate markers, including, inter alia, other polypeptides/polynucleotides of this invention. In some embodiments, such use may be in combination with other known markers for lung cancer, including but not limited to CEA, CA15-3, Beta-2-microglobulin, CA19-9, TPA, and/or in combination with native sequences associated with the polypeptides/polynucleotides of this invention, as herein described.

In some embodiments, the polypeptides/polynucleotides of this invention may be useful in, inter alia, assessing the presence, risk and/or extent of the following:
1. The identification of a metastasis of unknown origin which originated from a primary lung cancer.
2. The assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors.
3. Distinguishing between different types of lung cancer, therefore potentially affect treatment choice (e.g. small cell vs. non small cell tumors).
4. Unexplained dyspnea and/or chronic cough and/or hemoptysis, and analysis thereof.
5. Differential diagnosis of the origin of a pleural effusion.
6. Conditions which have similar symptoms, signs and complications as lung cancer and where the differential diagnosis between them and lung cancer is of clinical importance including but not limited to:
   a. Non-malignant causes of lung symptoms and signs. Symptoms and signs include, but are not limited to: lung lesions and infiltrates, wheeze, stridor.
   b. Other symptoms, signs and complications suggestive of lung cancer, such as tracheal obstruction, esophageal compression, dysphagia, recurrent laryngeal nerve paralysis, hoarseness, phrenic nerve paralysis with elevation of the hemidiaphragm and Horner syndrome.
   c. Any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.
7. Prediction of patient's drug response
8. As surrogate markers for clinical outcome of a treated cancer.
9. Screening for early detection of lung cancer.
   Renal Disease, and/or Condition

In one embodiment, the polypeptides and/or polynucleotides of this invention are utilized alone, or in combination with other markers, for the diagnosis, treatment selection and monitoring, or assessment of prognosis of any type of renal disease, and/or condition including but not limited to any type of renal damage; renal cancer, including but not limited to renal cell carcinoma; polycystic kidney disease; Diabetes induced nephropathy; Chronic Kidney Disease; Total Kidney Failure; Autoimmune nephropathy, including but not limited to Systemic lupus erythematosus (SLE), Goodpasture's syndrome, IgA nephropathy; Hereditary Nephritis—(Alport Syndrome); Infection-related Glomerular Disease, including but not limited to Acute post-streptococcal glomerulonephritis (PSGN), Bacterial endocarditis, HIV; Glomerulosclerosis; Focal segmental glomerulosclerosis (FSGS); Membranous nephropathy; Minimal change disease (MCD).

In some embodiments, the polypeptides/polynucleotides of this invention are utilized in conjunction with other screening procedures, as well as the use of other markers, for the diagnosis, or assessment of prognosis of renal disease and/or in a subject. In some embodiment, such markers and/or screening procedures may comprise urinary protein, creatinine or creatinine clearance. In another embodiments, the other markers may comprise markers used for the diagnosis or assessment of prognosis of renal cancer, specifically of renal cell carcinoma, including but not limited to vascular endothelial growth factor, interleukin-12, the soluble interleukin-2 receptor, intercellular adhesion molecule-1, human chorionic gonadotropin beta, insulin-like growth factor-1 receptor, Carbonic anhydrase 9 (CA 9), endostatin, Thymidine phosphorylase or combinations thereof.

In some embodiments, the polypeptides/polynucleotides of this invention may be useful in the diagnosis, treatment and/or assessment of prognosis of renal diseases and/or conditions. According to this aspect and in one embodiment, the polypeptides/polynucleotides useful in this context are: cluster AA340453 variants, cluster AA703666 variants, AI590292 variants, cluster HUMUMOD variants, or homologues thereof. In some embodiments, these polypeptides/polynucleotides are used alone or in combination with one or more other polypeptides/polynucleotides of this invention, and/or in combination with other markers known to detect kidney-related disorders, and/or screening procedures, including, inter alia, urinary protein, creatinine or creatinine clearance, and/or a native protein associated with the polypeptides of this invention, for example, native proteins of which the polypeptides are variants thereof.

### Candidate Marker Examples Section

This section relates to examples of sequences according to the present invention, including illustrative methods of selection thereof.

The markers of the present invention were tested with regard to their expression in various cancerous and non-cancerous tissue samples. A description of the samples used in the ovarian cancer testing panel is provided in Table 1_{—}1 below. A description of the samples used in the colon cancer testing panel is provided in Table 1_{—}2 below. A description of the samples used in the lung cancer testing panel is provided in Table 1_{—}3 below. A description of the samples used in the breast cancer testing panel is provided in Table 1_{—}4 below. A description of the samples used in the normal tissue panel, used also for the testing of the markers of the present invention with regard to their expression in various heart and non-heart tissue samples, and with regard to their expression in various kidney and non-kidney tissue samples, is provided in Table 1_{—}5 and 1_{—}6 below. Tests were then performed as described in the “Materials and Experimental Procedures” section below.

TABLE 1_1
Tissue samples in ovarian cancer testing panel

| | | | | | | gender/ | |
|---|---|---|---|---|---|---|---|
| Sample name | Lot number | Source | Tissue | Pathology | Grade | age | age |
| 33-B-Pap | A503175 | BioChain | ovary | Serous | 1 | 41/F | 41 |
| Sero CystAde | | | | papillary | | | |
| G1 | | | | cystadenocarcinoma | | | |
| 41-G-Mix | 98-03- | GOG | ovary | Mixed | 2 | 38 | 38 |
| Sero/Muc/Endo | G803 | | | epithelial | | | |
| G2 | | | | cystadenocarcinoma | | | |
| | | | | with | | | |
| | | | | mucinous, | | | |
| | | | | endometrioid, | | | |
| | | | | squamous | | | |
| | | | | and | | | |
| | | | | papillary | | | |
| | | | | serous | | | |
| | | | | (Stage2) | | | |
| 35-G-Endo | 94-08-7604 | GOG | right | Endometrioid | 2 | 39/F | 39 |
| Adeno G2 | | | ovary | adenocarcinoma | | | |
| 14-B-Adeno | A501111 | BioChain | ovary | Adenocarcinoma | 2 | 41/F | 41 |
| G2 | | | | | | | |
| 12-B-Adeno | A406023 | Biochain | ovary | Adenocarcinoma | 3 | 45/F | 45 |
| G3 | | | | | | | |
| 40-G-Mix | 95-11- | GOG | ovary, endometrium | Papillary | 2 | 49/F | 49 |
| Sero/Endo | G006 | | | serous and | | | |
| G2 | | | | endometrioid | | | |
| | | | | cystadenocarcinoma | | | |
| | | | | (Stage3C) | | | |
| 4-A-Pap | ILS-7286 | ABS | ovary | Papillary | 2 | 50/F | 50 |
| CystAdeno | | | | cystadenocarcinoma | | | |
| G2 | | | | | | | |
| 3-A-Pap | ILS-1431 | ABS | ovary | Papillary | 2 | 52/F | 52 |
| Adeno G2 | | | | adenocarcinoma | | | |
| 2-A-Pap | ILS-1408 | ABS | ovary | Papillary | 2 | 53/F | 53 |
| Adeno G2 | | | | adenocarcinoma | | | |
| 5-G-Adeno | 99-12- | GOG | ovary | Adenocarcinoma | 3 | 46/F | 46 |
| G3 | G432 | | | (Stage3C) | | | |
| 11-B-Adeno | A407068 | Biochain | ovary | Adenocarcinoma | 3 | 49/F | 49 |
| G3 | | | | | | | |
| 39--G-Mix | 2001-12- | GOG | ovary | Mixed | 3 | F | 49 |
| Sero/Endo | G037 | | | serous and | | | |
| G3 | | | | endometrioid | | | |
| | | | | adenocarcinoma | | | |
| 29-G-Sero | 2001-12- | GOG | right | Serous | 3 | 50/F | 50 |
| Adeno G3 | G035 | | ovary | adenocarcinoma | | | |
| | | | | (Stage3A) | | | |
| 70-G-Pap | 95-08- | GOG | ovary | Papillary | 3 | F/50 | 50 |
| Sero Adeno | G069 | | | serous | | | |
| G3 | | | | adenocarcinoma | | | |
| 6-A-Adeno | A0106 | ABS | ovary | adenocarcinoma | 3 | 51/F | 51 |
| G3 | | | | | | | |
| 31-B-Pap | A503176 | BioChain | ovary | Serous | 3 | 52/F | 52 |
| Sero CystAde | | | | papillary | | | |
| G3 | | | | cystadenocarcinoma | | | |
| 25-A-Pap | N0021 | ABS | ovary | Papillary | 3 | 55/F | 55 |
| Sero Adeno | | | | serous | | | |
| G3 | | | | adenocarcinoma | | | |
| | | | | (StageT3C | | | |
| | | | | N1MX) | | | |
| 37-G-Mix | 2002-05- | GOG | ovary | Mixed | 3 | 56/F | 56 |
| Sero/Endo | G513 | | | serous and | | | |
| G3 | | | | endometrioid | | | |
| | | | | adenocarcinoma | | | |
| 7-A-Adeno | IND-00375 | ABS | ovary | adenocarcinoma | 3 | 59/F | 59 |
| G3 | | | | | | | |
| 8-B-Adeno | A501113 | BioChain | ovary | adenocarcinoma | 3 | 60/F | 60 |
| G3 | | | | | | | |
| 10-B-Adeno | A407069 | Biochain | ovary | Adenocarcinoma | 3 | 60/F | 60 |
| G3 | | | | | | | |
| 38-G-Mix | 2002-05- | GOG | ovary | Mixed | 3 | 64/F | 64 |
| Sero/Endo | G509 | | | serous and | | | |
| G3 | | | | endometrioid | | | |
| | | | | adenocarcinoma | | | |
| | | | | of | | | |
| | | | | mullerian | | | |
| | | | | (Stage3C) | | | |
| 13-G-Adeno | 94-05-7603 | GOG | right | Poorly | 3 | 67/F | 67 |
| G3 | | | ovary | differentiated | | | |
| | | | | adenocarcinoma | | | |
| | | | | from | | | |
| | | | | primary | | | |
| | | | | peritoneal | | | |
| 24-G-Pap | 2001-07- | GOG | ovary | Papillary | 3 | 68/F | 68 |
| Sero Adeno | G801 | | | serous | | | |
| G3 | | | | adenocarcinoma | | | |
| 34-G-Pap | 95-04-2002 | GOG | ovary | Papillary | 3 | 68/F | 68 |
| Endo Adeno | | | | endometrioid | | | |
| G3 | | | | adenocarcinoma | | | |
| | | | | (Stage3C) | | | |
| 30-G-Pap | 2001-08- | GOG | ovary | Papillary | 3 | 72/F | 72 |
| Sero Adeno | G011 | | | serous | | | |
| G3 | | | | carcinoma | | | |
| | | | | (Stage1C) | | | |
| 1-A-Pap | ILS-1406 | ABS | ovary | Papillary | 3 | 73/F | 73 |
| Adeno G3 | | | | adenocarcinoma | | | |
| 9-G-Adeno | 99-06- | GOG | ovary | Adenocarcinoma | 3 | 84/F | 84 |
| G3 | G901 | | | (maybe | | | |
| | | | | serous) | | | |
| 32-G-Pap | 93-09-4901 | GOG | ovary | Serous | 3 | F | 67 |
| Sero CystAde | | | | papillary | | | |
| G3 | | | | cystadenocarcinoma | | | |
| 66-G-Pap | 2000-01- | GOG | ovary | Papillary | 3 | F | 67 |
| Sero Adeno | G413 | | | serous | | | |
| G3 SIV | | | | carcinoma | | | |
| | | | | (metastais | | | |
| | | | | of primary | | | |
| | | | | peritoneum) | | | |
| | | | | (Stage4) | | | |
| 19-B-Muc | A504085 | BioChain | ovary | Mucinous | 3 | 34/F | 34 |
| Adeno G3 | | | | adenocarcinoma | | | |
| 21-G-Muc | 95-10- | GOG | ovary | Mucinous | 2-3 | 44/F | 44 |
| CystAde G2-3 | G020 | | | cystadenocarcinoma | | | |
| | | | | (Stage2) | | | |
| 18-B-Muc | A504083 | BioChain | ovary | Mucinous | 3 | 45/F | 45 |
| Adeno G3 | | | | adenocarcinoma | | | |
| 20-A-Pap | USA- | ABS | ovary | Papillary | ?? | 46/F | 46 |
| Muc CystAde | 00273 | | | mucinous | | | |
| | | | | cystadenocarcinoma | | | |
| 17-B-Muc | A504084 | BioChain | ovary | Mucinous | 3 | 51/F | 51 |
| Adeno G3 | | | | adenocarcinoma | | | |
| 22-A-Muc | A0139 | ABS | ovary | Mucinous | 2 | 72/F | 72 |
| CystAde G2 | | | | cystadenocarcinoma | | | |
| | | | | (Stage1C) | | | |
| 43-G-Clear | 2001-10- | GOG | ovary | Clear cell | 3 | 74/F | 74 |
| cell Adeno | G002 | | | adenocarcinoma | | | |
| G3 | | | | | | | |
| 44-G-Clear | 2001-07- | GOG | ovary | Clear cell | | 73/F | 73 |
| cell Adeno | G084 | | | adenocarcinoma | | | |
| | | | | (Stage3A) | | | |
| 15-B-Adeno | A407065 | BioChain | ovary | Carcinoma | 3 | 27/F | 27 |
| G3 | | | | | | | |
| 16-Ct-Adeno | 1090387 | Clontech | ovary | Carcinoma | NA | 58/F | 58 |
| | | | | NOS | | | |
| 23-A-Muc | VNM- | ABS | ovary | Mucinous | 3 | 45/F | 45 |
| CystAde G3 | 00187 | | | cystadenocarcinoma | | | |
| | | | | with low | | | |
| | | | | malignant | | | |
| 42-G-Adeno | 98-08- | GOG | ovary | Epithelial | | 46/F | 46 |
| borderline | G001 | | | adenocarcinoma | | | |
| | | | | of | | | |
| | | | | borderline | | | |
| | | | | malignancy | | | |
| 63-G-Sero | 2000-10- | GOG | ovary | Serous | | 71/F | 71 |
| CysAdenoFibroma | G620 | | | CysAdeno | | | |
| | | | | Fibroma of | | | |
| | | | | borderline | | | |
| | | | | malignancy | | | |
| 62-G-Ben | 99-10- | GOG | ovary | Benbin | | 32/F | 32 |
| Muc | G442 | | | mucinus | | | |
| CysAdenoma | | | | cysadenoma | | | |
| 60-G-Muc | 99-01- | GOG | ovary | Mucinous | | 40/F | 40 |
| CysAdenoma | G043 | | | Cysadenoma | | | |
| 56-G-Ben | 99-01- | GOG | left | Bengin | | 46/F | 46 |
| Muc | G407 | | ovary | mucinus | | | |
| CysAdeno | | | | cysadenoma | | | |
| 64-G-Ben | 99-06- | GOG | ovary | Bengin | | 57/F | 57 |
| Sero | G039 | | | Serous | | | |
| CysAdenoma | | | | CysAdenoma | | | |
| 61-G-Muc | 99-07- | GOG | ovary | Mucinous | | 63/F | 63 |
| CysAdenoma | G011 | | | Cysadenoma | | | |
| 59-G-Sero | 98-12- | GOG | ovary | Serous | | 77/F | 77 |
| CysAdenoFibroma | G401 | | | CysAdeno | | | |
| | | | | Fibroma | | | |
| 51-G-N M41 | 98-03- | GOG | ovary | Normal | | 38/F | 38 |
| | G803N | | | (matched | | | |
| | | | | tumor 98- | | | |
| | | | | 03-G803) | | | |
| 75-G-N M60 | 99-01- | GOG | ovary | Normal | | 40/F | 40 |
| | G043N | | | (matched | | | |
| | | | | tumor 99- | | | |
| | | | | 01-G043) | | | |
| 49-B-N M14 | A501112 | BioChain | ovary | Normal | | 41/F | 41 |
| | | | | (matched | | | |
| | | | | tumor | | | |
| | | | | A501111) | | | |
| 52-G-N M42 | 98-08- | GOG | ovary | Normal | | 46/F | 46 |
| | G001N | | | (matched | | | |
| | | | | tumor 98- | | | |
| | | | | 08-G001) | | | |
| 68-G-N M56 | 99-01- | GOG | ovary | Normal | | 46/F | 46 |
| | G407N | | | (matched | | | |
| | | | | bengin 99- | | | |
| | | | | 01-G407) | | | |
| 50-B-N M8 | A501114 | BioChain | ovary | Normal | | 60/F | 60 |
| | | | | (matched | | | |
| | | | | tumor | | | |
| | | | | A501113) | | | |
| 67-G-N M38 | 2002-05- | GOG | ovary | Normal | | 64/F | 64 |
| | 509N | | | (matched | | | |
| | | | | tumor | | | |
| | | | | 2002-05- | | | |
| | | | | G509) | | | |
| 69-G-N M24 | 2001-07- | GOG | ovary | Normal | | 68/F | 68 |
| | G801N | | | (matched | | | |
| | | | | tumor | | | |
| | | | | 2001-07- | | | |
| | | | | G801) | | | |
| 73-G-N M59 | 98-12- | GOG | ovary | Normal | | 77/F | 77 |
| | G401N | | | (matched | | | |
| | | | | tumor 98- | | | |
| | | | | 12-G401) | | | |
| 72-G-N M66 | 2000-01- | GOG | ovary | Normal | | F | F |
| | G413N | | | (matched | | | |
| | | | | tumor | | | |
| | | | | 2000-01- | | | |
| | | | | G413) | | | |
| 45-B-N | A503274 | BioChain | ovary | Normal | | 41/F | 41 |
| | | | | PM | | | |
| 46-B-N | A504086 | BioChain | ovary | Normal | | 41/F | 41 |
| | | | | PM | | | |
| 71-CG-N | CG-188-7 | Ichilov | ovary | Normal | | 49/F | 49 |
| | | | | PM | | | |
| 48-B-N | A504087 | BioChain | ovary | Normal | | 51/F | 51 |
| | | | | PM | | | |

TABLE 1_2
Tissue samples in colon cancer testing panel

| | | | | | | gender/ |
|---|---|---|---|---|---|---|
| sample rename | Lot No. | tissue | source | pathology | Grade | age |
| 58-B-Adeno G1 | A609152 | Colon | biochain | Adenocarcinoma | 1 | M/73 |
| 59-B-Adeno G1 | A609059 | Colon | biochain | Adenocarcinoma, | 1 | M/58 |
| | | | | Ulcer | | |
| 14-CG-Polypoid | CG-222 (2) | Rectum | Ichilov | Well polypoid | | F/49 |
| Adeno G1 D-C | | | | adeocarcinoma | | |
| | | | | Duke's C | | |
| 17-CG-Adeno | CG-163 | Rectum | Ichilov | Adenocarcinoma | 2 | M/73 |
| G1-2 | | | | | | |
| 10-CG-Adeno | CG-311 | Sigmod | Ichilov | Adenocarcinoma | 1-2 | M/88 |
| G1-2 D-B2 | | colon | | Astler-Coller B2. | | |
| 11-CG-Adeno | CG-337 | Colon | Ichilov | Adenocarcinoma | 1-2 | NA |
| G1-2 D-C2 | | | | Astler-Coller C2. | | |
| 6-CG-Adeno G1- | CG-303 (3) | Colon | Ichilov | Adenocarcinoma | 1-2 | F/77 |
| 2 D-C2 | | | | Astler-Coller C2. | | |
| 5-CG-Adeno G2 | CG-308 | Colon | Ichilov | Adenocarcinoma. | 2 | F/80 |
| | | Sigma | | | | |
| 16-CG-Adeno G2 | CG-278C | colon | Ichilov | Adenocarcinoma | 2 | F/60 |
| 56-B-Adeno G2 | A609148 | Colon | biochain | Adenocarcinoma | 2 | F48 |
| 61-B-Adeno G2 | A606258 | Colon | biochain | Adenocarcinoma, | 2 | M/41 |
| | | | | Ulcer | | |
| 60-B-Adeno G2 | A609058 | Colon | biochain | Adenocarcinoma, | 2 | M/67 |
| | | | | Ulcer | | |
| 22-CG-Adeno G2 | CG-229C | Colon | Ichilov | Adenocarcinoma | 2 | F/55 |
| D-B | | | | Duke's B | | |
| 1-CG-Adeno G2 | CG-335 | Cecum | Ichilov | Adenocarcinoma | 2 | F/66 |
| D-B2 | | | | Dukes B2. | | |
| 12-CG-Adeno G2 | CG-340 | Colon | Ichilov | Adenocarcinoma | 2 | M/66 |
| D-B2 | | Sigma | | Astler-Coller B2. | | |
| 28-CG-Adeno G2 | CG-284 | sigma | Ichilov | Adenocarcinoma | 2 | F/72 |
| D-B2 | | | | Duke's B2 | | |
| 2-CG-Adeno G2 | CG-307 X2 | Cecum | Ichilov | Adenocarcinoma | 2 | F/89 |
| D-C2 | | | | Astler-Coller C2. | | |
| 9-CG-Adeno G2 | CG-297 X2 | Rectum | Ichilov | Adenocarcinoma | 2 | M/62 |
| D-D | | | | Dukes D. | | |
| 13-CG-Adeno G2 | CG-290 X2 | Rectosigmoidal | Ichilov | Adenocarcinoma | 2 | M/47 |
| D-D | | colon | | Dukes D. | | |
| 26-CG-Adeno G2 | CG-283 | sigma | Ichilov | Colonic | 2 | F/63 |
| D-D | | | | adenocarcinoma | | |
| | | | | Duke's D | | |
| 4-CG-Adeno G3 | CG-276 | Colon | Ichilov | Carcinoma. | 3 | M/64 |
| 53-B-Adeno G3 | A609161 | Colon | biochain | Adenocarcinoma | 3 | F/53 |
| 54-B-Adeno G3 | A609142 | Colon | biochain | Adenocarcinoma | 3 | M/53 |
| 55-B-Adeno G3 | A609144 | Colon | biochain | Adenocarcinoma | 3 | M/68 |
| 57-B-Adeno G3 | A609150 | Colon | biochain | Adenocarcinoma | 3 | F/45 |
| 72-CG-Adeno G3 | CG-309 | colon | Ichilov | Adenocarcinoma | 3 | F/88 |
| 20-CG-Adeno G3 | CG-249 | Colon | Ichilov | Ulcerated | 3 | M/36 |
| D-B2 | | | | adenocarcinoma | | |
| | | | | Duke's B2 | | |
| 7-CG-Adeno D-A | CG-235 | Rectum | Ichilov | Adenocarcinoma | UN | F/66 |
| | | | | intramucosal | | |
| | | | | Duke's A. | | |
| 23-CG-Adeno D-C | CG-282 | sigma | Ichilov | Mucinus | UN | M/51 |
| | | | | adenocarcinoma | | |
| | | | | Astler Coller C | | |
| 3-CG-Muc adeno | CG-224 | Colon | Ichilov | Mucinois | UN | M/48 |
| D-D | | | | adenocarcinoma | | |
| | | | | Duke's D | | |
| 18-CG-Adeno | CG-22C | Colon | Ichilov | Adenocarcinoma | UN | NA |
| 19-CG-Adeno | CG-19C (1) | Colon | Ichilov | Adenocarcinoma | UN | NA |
| 21-CG-Adeno | CG-18C | Colon | Ichilov | Adenocarcinoma | UN | NA |
| 24-CG-Adeno | CG-12 (2) | Colon | Ichilov | Adenocarcinoma | UN | NA |
| 25-CG-Adeno | CG-2 | Colon | Ichilov | Adenocarcinoma | UN | NA |
| 27-CG-Adeno | CG-4 | Colon | Ichilov | Adenocarcinoma | UN | NA |
| 8-CG- | CG-291 | Wall of | Ichilov | Diverticolosis and | | F/65 |
| diverticolosis, | | sigma | | diverticulitis of the | | |
| diverticulitis | | | | Colon | | |
| 46-CG-Crohn's | CG-338C | Cecum | Ichilov | Crohn's disease | | M/22 |
| disease | | | | | | |
| 47-CG-Crohn's | CG-338AC | Colon | Ichilov | Crohn's disease. | | M/22 |
| disease | | | | | | |
| 42-CG-N M20 | CG-249N | Colon | Ichilov | Normal | | M/36 |
| 43-CG-N M8 | CG-291N | Wall of | Ichilov | Normal | | F/65 |
| | | sigma | | | | |
| 44-CG-N M21 | CG-18N | Colon | Ichilov | Normal | | NA |
| 45-CG-N M11 | CG-337N | Colon | Ichilov | Normal | | M/75 |
| 49-CG-N M14 | CG-222N | Rectum | Ichilov | Normal | | F/49 |
| 50-CG-N M5 | CG-308N | Sigma | Ichilov | Within normal | | F/80 |
| | | | | limits | | |
| 51-CG-N M26 | CG-283N | Sigma | Ichilov | Normal | | F/63 |
| 41-B-N | A501156 | Colon | biochain | Normal PM | | M/78 |
| 52-CG-N | CG-309TR | Colon | Ichilov | Within normal | | F/88 |
| | | | | limits | | |
| 62-B-N | A608273 | Colon | biochain | Normal PM | | M/66 |
| 63-B-N | A609260 | Colon | biochain | Normal PM | | M/61 |
| 64-B-N | A609261 | Colon | biochain | Normal PM | | F/68 |
| 65-B-N | A607115 | Colon | biochain | Normal PM | | M/24 |
| 66-B-N | A609262 | Colon | biochain | Normal PM | | M/58 |
| 67-B-N | A406029 | Colon | biochain | Normal PM (Pool | | |
| | | | | 10) | | |
| 69-B-N | A411078 | Colon | biochain | Normal PM (Pool | | F&M |
| | | | | 10) | | |
| 70-Cl-N | 1110101 | Colon | clontech | Normal PM (Pool | | |
| | | | | of 3) | | |
| 71-Am-N | 071P10B | Colon | Ambion | Normal (IC | | F/34 |
| | | | | BLEED) | | |

TABLE 1_3
Tissue samples in lung cancer testing panel

| sample rename | Lot No. | source | pathology | Grade | gender/age |
|---|---|---|---|---|---|
| 1-B-Adeno G1 | A504117 | Biochain | Adenocarcinoma | 1 | F/29 |
| 2-B-Adeno G1 | A504118 | Biochain | Adenocarcinoma | 1 | M/64 |
| 95-B-Adeno G1 | A610063 | Biochain | Adenocarcinoma | 1 | F/54 |
| 12-B-Adeno G2 | A504119 | Biochain | Adenocarcinoma | 2 | F/74 |
| 75-B-Adeno G2 | A609217 | Biochain | Adenocarcinoma | 2 | M/65 |
| 77-B-Adeno G2 | A608301 | Biochain | Adenocarcinoma | 2 | M/44 |
| 13-B-Adeno G2-3 | A504116 | Biochain | Adenocarcinoma | 2-3 | M/64 |
| 89-B-Adeno G2-3 | A609077 | Biochain | Adenocarcinoma | 2-3 | M/62 |
| 76-B-Adeno G3 | A609218 | Biochain | Adenocarcinoma | 3 | M/57 |
| 94-B-Adeno G3 | A610118 | Biochain | Adenocarcinoma | 3 | M/68 |
| 3-CG-Adeno | CG-200 | Ichilov | Adenocarcinoma | | NA |
| 14-CG-Adeno | CG-111 | Ichilov | Adenocarcinoma | | M/68 |
| 15-CG-Bronch adeno | CG-244 | Ichilov | Bronchioloalveolar | | M/74 |
| | | | adenocarcinoma | | |
| 45-B-Alvelous Adeno | A501221 | Biochain | Alveolus carcinoma | | F/50 |
| 44-B-Alvelous Adeno | A501123 | Biochain | Alveolus carcinoma | 2 | F/61 |
| G2 | | | | | |
| 19-B-Squamous G1 | A408175 | Biochain | Squamous carcinoma | 1 | M/78 |
| 16-B-Squamous G2 | A409091 | Biochain | Squamous carcinoma | 2 | F/68 |
| 17-B-Squamous G2 | A503183 | Biochain | Squamous carcinoma | 2 | M/57 |
| 21-B-Squamous G2 | A503187 | Biochain | Squamous carcinoma | 2 | M/52 |
| 78-B-Squamous G2 | A607125 | Biochain | Squamous Cell | 2 | M/62 |
| | | | Carcinoma | | |
| 80-B-Squamous G2 | A609163 | Biochain | Squamous Cell | 2 | M/74 |
| | | | Carcinoma | | |
| 18-B-Squamous G2-3 | A503387 | Biochain | Squamous Cell | 2-3 | M/63 |
| | | | Carcinoma | | |
| 81-B-Squamous G3 | A609076 | Biochain | Squamous Carcinoma | 3 | m/53 |
| 79-B-Squamous G3 | A609018 | Biochain | Squamous Cell | 3 | M/67 |
| | | | Carcinoma | | |
| 20-B-Squamous | A501121 | Biochain | Squamous Carcinoma | | M/64 |
| 22-B-Squamous | A503386 | Biochain | Squamous Carcinoma | | M/48 |
| 88-B-Squamous | A609219 | Biochain | Squamous Cell | | M/64 |
| | | | Carcinoma | | |
| 100-B-Squamous | A409017 | Biochain | Squamous Carcinoma | | M/64 |
| 23-CG-Squamous | CG-109 (1) | Ichilov | Squamous Carcinoma | | M/65 |
| 24-CG-Squamous | CG-123 | Ichilov | Squamous Carcinoma | | M/76 |
| 25-CG-Squamous | CG-204 | Ichilov | Squamous Carcinoma | | M/72 |
| 87-B-Large cell G3 | A609165 | Biochain | Large Cell Carcinoma | 3 | F/47 |
| 38-B-Large cell | A504113 | Biochain | Large cell | | M/58 |
| 39-B-Large cell | A504114 | Biochain | Large cell | | F/35 |
| 82-B-Large cell | A609170 | Biochain | Large Cell | | M/68 |
| | | | Neuroendocrine | | |
| | | | Carcinoma | | |
| 30-B-Small cell carci | A501389 | Biochain | small cell | 3 | M/34 |
| G3 | | | | | |
| 31-B-Small cell carci | A501390 | Biochain | small cell | 3 | F/59 |
| G3 | | | | | |
| 32-B-Small cell carci | A501391 | Biochain | small cell | 3 | M/30 |
| G3 | | | | | |
| 33-B-Small cell carci | A504115 | Biochain | small cell | 3 | M |
| G3 | | | | | |
| 86-B-Small cell carci | A608032 | Biochain | Small Cell Carcinoma | 3 | F/52 |
| G3 | | | | | |
| 83-B-Small cell carci | A609162 | Biochain | Small Cell Carcinoma | | F/47 |
| 84-B-Small cell carci | A609167 | Biochain | Small Cell Carcinoma | | F/59 |
| 85-B-Small cell carci | A609169 | Biochain | Small Cell Carcinoma | | M/66 |
| 46-B-N M44 | A501124 | Biochain | Normal M44 | | F/61 |
| 47-B-N | A503205 | Biochain | Normal PM | | M/26 |
| 48-B-N | A503206 | Biochain | Normal PM | | M/44 |
| 49-B-N | A503384 | Biochain | Normal PM | | M/27 |
| 50-B-N | A503385 | Biochain | Normal PM | | M/28 |
| 90-B-N | A608152 | Biochain | Normal (Pool 2) PM | | pool 2 |
| 91-B-N | A607257 | Biochain | Normal (Pool 2) PM | | pool 2 |
| 92-B-N | A503204 | Biochain | Normal PM | | m/28 |
| 93-Am-N | 111P0103A | Ambion | Normal PM | | F/61 |
| 96-Am-N | 36853 | Ambion | Normal PM | | F/43 |
| 97-Am-N | 36854 | Ambion | Normal PM | | M/46 |
| 98-Am-N | 36855 | Ambion | Normal PM | | F/72 |
| 99-Am-N | 36856 | Ambion | Normal PM | | M/31 |

TABLE 1_4
Tissue samples in breast cancer testing panel

| sample rename | Lot no | source | pathology | grade | age | TNM | stage |
|---|---|---|---|---|---|---|---|
| 14-A-IDC G2 | A0135T | ABS | IDC | 2 | 37 | T2N2Mx | |
| 43-B-IDC G2 | A609183 | Biochain | IDC | 2 | 40 | | |
| 54-B-IDC G2 | A605353 | Biochain | IDC | 2 | 41 | | |
| 55-B-IDC G2 | A609179 | Biochain | IDC | 2 | 42 | | |
| 47-B-IDC G2 | A609221 | Biochain | IDC | 2 | 42 | | |
| 17-A-IDC G2 | 4904020036T | ABS | IDC | 2-3 | 42 | T3N1Mx | |
| 42-A-IDC G3 | 6005020031T | ABS | IDC | 3 | 42 | T1cN0Mx | |
| 7-A-IDC G2 | 7263T | ABS | IDC | 2 | 43 | T1N0M0 | stage 1 |
| 48-B-IDC G2 | A609222 | Biochain | IDC | 2 | 44 | | |
| 53-B-IDC G2 | A605151 | Biochain | IDC | 2 | 44 | | |
| 12-A-IDC G2 | 1432T | ABS | IDC | 2 | 46 | T2N0M0 | stage 2A |
| 61-B-IDC G2 | A610029 | Biochain | IDC | 2 | 46 | | |
| 46-B-Carci G2 | A609177 | Biochain | Carcinoma | 2 | 48 | | |
| 16-A-IDC G2 | 4904020032T | ABS | IDC | 2 | 49 | T3N1Mx | |
| 62-B-IDC G2 | A609194 | Biochain | IDC | 2 | 51 | | |
| 49-B-IDC G2 | A609223 | Biochain | IDC | 2 | 54 | | |
| 32-A-Muc | 7116T | ABS | Mucinous | | 54 | T2N0M0 | stage 2A |
| Carci | | | carcinoma | | | | |
| 45-B-IDC G2 | A609181 | Biochain | IDC | 2 | 58 | | |
| 15-A-IDC G2 | 7259T | ABS | IDC | 2 | 59 | T3N1M0 | stage 3A |
| 52-B-ILC G1 | A605360 | Biochain | Invasive | 1 | 60 | | |
| | | | Lobular | | | | |
| | | | Carcinoma | | | | |
| 6-A-IDC G1 | 7238T | ABS | IDC | 1 | 60 | T2N0M0 | stage 2A |
| 26-A-IDC G3 | 7249T | ABS | IDC | 3 | 60 | T2N0M0 | stage 2A |
| 13-A-IDC G2 | A0133T | ABS | IDC | 2 | 63 | T2N1aMx | |
| 50-B-IDC G2 | A609224 | Biochain | IDC | 2 | 69 | | |
| 44-B-IDC G2 | A609198 | Biochain | IDC | 2 | 77 | | |
| 51-B-IDC G1 | A605361 | Biochain | IDC | 1 | 79 | | |
| 31-CG-IDC | CG-154 | Ichilov | IDC | | 83 | | |
| 27-A-IDC G3 | 4907020072T | ABS | IDC | 3 | 91 | T2N0Mx | |
| 36-A-N M7 | 7263N | ABS | Normal | | 43 | | |
| | | | matched to | | | | |
| | | | 7T | | | | |
| 40-A-N M12 | 1432N | ABS | Normal | | 46 | | |
| | | | matched to | | | | |
| | | | 12T | | | | |
| 39-A-N M15 | 7259N | ABS | Normal | | 59 | | |
| | | | matched to | | | | |
| | | | 15T | | | | |
| 35-A-N M6 | 7238N | ABS | Normal | | 60 | | |
| | | | matched to | | | | |
| | | | 6T | | | | |
| 41-A-N M26 | 7249N | ABS | Normal | | 60 | | |
| | | | matched to | | | | |
| | | | 26T | | | | |
| 57-B-N | A609233 | Biochain | Normal PM | | 34 | | |
| 59-B-N | A607155 | Biochain | Normal PM | | 35 | | |
| 60-B-N | A609234 | Biochain | Normal PM | | 36 | | |
| 63-Am-N | 26486 | Ambion | Normal PS | | 43 | | |
| 66-Am-N | 36678 | Ambion | Normal PM | | 45 | | |
| 64-Am-N | 23036 | Ambion | Normal PM | | 57 | | |
| 56-B-N | A609235 | Biochain | Normal PM | | 59 | | |
| 65-Am-N | 31410 | Ambion | Normal PM | | 63 | | |
| 67-Am-N | 073P010602086A | Ambion | Normal PM | | 64 | | |
| 58-B-N | A609232 | Biochain | Normal PM | | 65 | | |

TABLE 1_5
Tissue samples in normal panel:

| | Lot no. | Source | Tissue | Pathology | Sex/Age | comments |
|---|---|---|---|---|---|---|
| 1-Am- | 071P10B | Ambion | Colon | PM IC bleed | F/43 | IC- |
| Colon (C71) | | | | | | intracarnial |
| | | | | | | bleed |
| 2-B-Colon | A411078 | Biochain | Colon | PM-Pool of 10 | M (26-78) | |
| (C69) | | | | | &F (53-77) | |
| 3-Cl-Colon | 1110101 | Clontech | Colon | PM-Pool of 3 | M&F | |
| (C70) | | | | sudden death | (20-50) | |
| 4-Am-Small | 091P0201A | Ambion | Small | PM ICH | M/85 | |
| Intestine | | | Intestine | | | |
| 5-B-Small | A501158 | Biochain | Small | PM | M/63 | |
| Intestine | | | Intestine | | | |
| 6-B-Rectum | A605138 | Biochain | Rectum | PM | M/25 | |
| 7-B-Rectum | A610297 | Biochain | Rectum | PM | M/24 | |
| 8-B-Rectum | A610298 | Biochain | Rectum | PM | M/27 | |
| 9-Am- | 110P04A | Ambion | Stomach | PM GSW | M/16 | |
| Stomach | | | | | | |
| 10-B- | A501159 | Biochain | Stomach | PM | M/24 | |
| Stomach | | | | | | |
| 11-B- | A603814 | Biochain | Esophagus | PM | M/26 | |
| Esophagus | | | | | | |
| 12-B- | A603813 | Biochain | Esophagus | PM | M/41 | |
| Esophagus | | | | | | |
| 13-Am- | 071P25C | Ambion | Pancreas | PM MVA | F/25 | |
| Pancreas | | | | | | |
| 14-CG- | CG-255-2 | Ichilov | Pancreas | PM | M/75 | |
| Pancreas | | | | | | |
| 15-B-Lung | A409363 | Biochain | Lung | PM-Pool of 5 | M (24-28) | |
| | | | | | &F62 | |
| 16-Am- | 111P0103A | Ambion | Lung | PM ICH | F/61 | |
| Lung (L93) | | | | | | |
| 17-B-Lung | A503204 | Biochain | Lung | PM | M/28 | |
| (L92) | | | | | | |
| 19-B-Ovary | A504087 | Biochain | Ovary | PM | F/51 | |
| (O48) | | | | | | |
| 20-B-Ovary | A504086 | Biochain | Ovary | PM | F/41 | |
| (O46) | | | | | | |
| 75-G-Ovary | L629FRV1 | GCI | Ovary | PS | F/47 | |
| | | | | DIGESTIVE | | |
| | | | | HEMORRHAGE | | |
| | | | | (ALCOHOLISM) | | |
| 76-G-Ovary | DWHTZRQX | GCI | Ovary | PS | F/42 | |
| | | | | LEIOMYOMAS | | |
| 77-G-Ovary | FDPL9NJ6 | GCI | Ovary | PS VAGINAL | F/56 | |
| | | | | BLEEDING | | |
| 78-G-Ovary | GWXUZN5M | GCI | Ovary | PS | F/53 | |
| | | | | ABNORMAL | | |
| | | | | PAP SMEARS | | |
| 21-Am- | 101P0101A | Ambion | Cervix | PM Surgery | F/40 | |
| Cervix | | | | | | |
| 23-B-Cervix | A504089 | Biochain | Cervix | PM-Pool of 5 | F (36-55) | |
| 24-B-Uterus | A411074 | Biochain | Uterus | PM-Pool of 10 | F (32-53) | |
| 25-B-Uterus | A409248 | Biochain | Uterus | PM | F/35 | |
| 26-B-Uterus | A504090 | Biochain | Uterus | PM-Pool of 5 | F (40-53) | |
| 28-Am- | 071P02C | Ambion | Bladder | PM GSW | M/28 | |
| Bladder | | | | | | |
| 29-B- | A504088 | Biochain | Bladder | PM-Pool of 5 | M (26-44) | |
| Bladder | | | | | &F30 | |
| 30-Am- | 021P33A | Ambion | Placenta | PB | F/33 | PB - |
| Placenta | | | | | | post |
| | | | | | | birth |
| 31-B- | A410165 | Biochain | Placenta | PB | F/26 | |
| Placenta | | | | | | |
| 32-B- | A411073 | Biochain | Placenta | PB-Pool of 5 | F (24-30) | |
| Placenta | | | | | | |
| 33-B-Breast | A607155 | Biochain | Breast | PM | F/36 | |
| (B59) | | | | | | |
| 34-Am- | 26486 | Ambion | Breast | PS bilateral | F/43 | |
| Breast | | | | breast | | |
| (B63) | | | | reduction | | |
| 35-Am- | 23036 | Ambion | Breast | PM lung cancer | F/57 | |
| Breast | | | | | | |
| (B64) | | | | | | |
| 36-Cl- | 1070317 | Clontech | Prostate | PM-Pool of 47 | M (14-57) | |
| Prostate | | | | sudden death | | |
| (P53) | | | | | | |
| 37-Am- | 061P04A | Ambion | Prostate | PM IC bleed | M/47 | |
| Prostate | | | | | | |
| (P42) | | | | | | |
| 38-Am- | 25955 | Ambion | Prostate | PM head | M/62 | |
| Prostate | | | | trauma | | |
| (P59) | | | | | | |
| 39-Am- | 111P0104A | Ambion | Testis | PM GSW | M/25 | |
| Testis | | | | | | |
| 40-B-Testis | A411147 | Biochain | Testis | PM | M/74 | |
| 41-Cl-Testis | 1110320 | Clontech | Testis | PM-Pool of 45 | M (14-64) | |
| | | | | sudden death | | |
| 42-CG- | CG-184-10 | Ichilov | Adrenal | PM | F/81 | |
| Adrenal | | | | | | |
| 43-B- | A610374 | Biochain | Adrenal | PM | F/83 | |
| Adrenal | | | | | | |
| 44-B-Heart | A411077 | Biochain | Heart | PM-Pool of 5 | M (23-70) | |
| 45-CG- | CG-255-9 | Ichilov | Heart | PM | M/75 | |
| Heart | | | focal | | | |
| | | | fibrosis | | | |
| 46-CG- | CG-227-1 | Ichilov | Heart | PM | F/36 | |
| Heart | | | | | | |
| 47-Am- | 081P0101A | Ambion | Liver | PM ICH | M/64 | |
| Liver | | | | | | |
| 48-CG- | CG-93-3 | Ichilov | Liver | PM | F/19 | |
| Liver | | | | | | |
| 49-CG- | CG-124-4 | Ichilov | Liver of | PM | fetus | |
| Liver | | | fetus | | | |
| 50-Cl-BM | 1110932 | Clontech | Bone | PM-Pool of 8 | M&F | |
| | | | Marrow | sudden death | (22-65) | |
| 51-CGEN- | WBC#5 | CGEN | Blood | — | M | |
| Blood | | | | | | |
| 52-CGEN- | WBC#4 | CGEN | Blood | — | M | |
| Blood | | | | | | |
| 53-CGEN- | WBC#3 | CGEN | Blood | — | M | |
| Blood | | | | | | |
| 54-CG- | CG-267 | Ichilov | Spleen | PM | F/25 | |
| Spleen | | | | | | |
| 55-CG- | 111P0106B | Ambion | Spleen | PM GSW | M/25 | |
| Spleen | | | | | | |
| 56-CG- | A409246 | Biochain | Spleen | PM | F/12 | |
| Spleen | | | | | | |
| 57-CG- | CG-98-7 | Ichilov | Thymus | PM | F/28 | |
| Thymus | | | | | | |
| 58-Am- | 101P0101A | Ambion | Thymus | PM head injury | M/14 | |
| Thymus | | | | | | |
| 59-B- | A409278 | Biochain | Thymus | PM | M/28 | |
| Thymus | | | | | | |
| 60-B- | A610287 | Biochain | Thyroid | PM | M/27 | |
| Thyroid | | | | | | |
| 61-B- | A610286 | Biochain | Thyroid | PM | M/24 | |
| Thyroid | | | | | | |
| 62-CG- | CG-119-2 | Ichilov | Thyroid | PM | F/66 | |
| Thyroid | | | | | | |
| 63-Cl- | 1070319 | Clontech | Salivary | PM-Pool of 24 | M&F 15-60 | |
| Salivary | | | Gland | sudden death | | |
| Gland | | | | | | |
| 64-Am- | 111P0101B | Ambion | Kidney | PM ICH | M 60 | |
| Kidney | | | | | | |
| 65-Cl- | 1110970 | Clontech | Kidney | PM-Pool of 14 | M&F 18-59 | |
| Kidney | | | | sudden death | | |
| 66-B- | A411080 | Biochain | Kidney | PM-Pool of 5 | M24-46 | |
| Kidney | | | | | | |
| 67-CG- | CG-183-5 | Ichilov | Cerebellum | PM | M/74 | |
| Cerebellum | | | | | | |
| 68-CG- | CG-212-5 | Ichilov | Cerebellum | PM | M/54 | |
| Cerebellum | | | | | | |
| 69-B-Brain | A411322 | Biochain | Brain | PM | M/28 | |
| 70-Cl-Brain | 1120022 | Clontech | Brain | PM | — | |
| 71-B-Brain | A411079 | Biochain | Brain | PM-Pool of 2 | M27-28 | |
| 72-CG- | CG-151-1 | Ichilov | Brain | PM | F/86 | |
| Brain | | | | | | |
| 73-Am- | 101P013A | Ambion | Skeletal | PM head injury | F/28 | |
| Skeletal | | | Muscle | | | |
| Muscle | | | | | | |
| 74-Cl- | 1061038 | Clontech | Skeletal | PM-Pool of 2 | M&F 43-46 | |
| Skeletal | | | Muscle | sudden death | | |
| Muscle | | | | | | |
| 18-Am- | 061P43A | Ambion | Ovary | PM | F/16 | |
| Ovary | | | | | | |
| (O47) | | | | | | |
| 22-B-Cervix | A408211 | Biochain | Cervix | PM | F/36 | |
| 27-B- | A501157 | Biochain | Bladder | PM | M/29 | |
| Bladder | | | | | | |

TABLE 1_6

| | | Sample | | |
|---|---|---|---|---|
| | | id (GCI)/case | Tissue id | Sample id |
| | | id (Asterand) | (GCI)/Specimen | (Asterand)/RNA |
| sample name | Source | Lot no. | id (Asternd) | id (GCI) |
| 1-(7)-Bc-Rectum | Biochain | A610297 | | |
| 2-(8)-Bc-Rectum | Biochain | A610298 | | |
| 3-GC-Colon | GCI | CDSUV | CDSUVNR3 | |
| 4-As-Colon | Asterand | 16364 | 31802 | 31802B1 |
| 5-As-Colon | Asterand | 22900 | 74446 | 74446B1 |
| 6-GC-Small bowl | GCI | V9L7D | V9L7DN6Z | |
| 7-GC-Small bowl | GCI | M3GVT | M3GVTN5R | |
| 8-GC-Small bowl | GCI | 196S2 | 196S2AJN | |
| 9-(9)-Am-Stomach | Ambion | 110P04A | | |
| 10-(10)-Bc- | Biochain | A501159 | | |
| Stomach | | | | |
| 11-(11)-Bc-Esoph | Biochain | A603814 | | |
| 12-(12)-Bc-Esoph | Biochain | A603813 | | |
| 13-As-Panc | Asterand | 8918 | 9442 | 9442C1 |
| 14-As-Panc | Asterand | 10082 | 11134 | 11134B1 |
| 15-(48)-Ic-Liver | Ichilov | CG-93-3 | | |
| 16-As-Liver | Asterand | 7916 | 7203 | 7203B1 |
| 17-(28)-Am- | Ambion | 071P02C | | |
| Bladder | | | | |
| 18-(29)-Bc-Bladder | Biochain | A504088 | | |
| 19-(64)-Am- | Ambion | 111P0101B | | |
| Kidney | | | | |
| 20-(65)-Cl-Kidney | Clontech | 1110970 | | |
| 21-(66)-Bc-Kidney | Biochain | A411080 | | |
| 22-GC-Kidney | GCI | N1EVZ | N1EVZN91 | |
| 23-GC-Kidney | GCI | BMI6W | BMI6WN9F | |
| 24-(42)-Ic-Adrenal | Ichilov | CG-184-10 | | |
| 25-(43)-Bc-Adrenal | Biochain | A610374 | | |
| 26-(16)-Am-Lung | Ambion | 111P0103A | | |
| 27-(17)-Bc-Lung | Biochain | A503204 | | |
| 28-As-Lung | Asterand | 9078 | 9275 | 9275B1 |
| 29-As-Lung | Asterand | 6692 | 6161 | 6161A1 |
| 30-As-Lung | Asterand | 7900 | 7180 | 7180F1 |
| 31-(75)-GC-Ovary | GCI | L629FRV1 | | |
| 32-(76)-GC-Ovary | GCI | DWHTZRQX | | |
| 33-(77)-GC-Ovary | GCI | FDPL9NJ6 | | |
| 34-(78)-GC-Ovary | GCI | GWXUZN5M | | |
| 35-(21)-Am-Cerix | Ambion | 101P0101A | | |
| 36-GC-cervix | GCI | E2P2N | E2P2NAP4 | |
| 37-(24)-Bc-Uterus | Biochain | A411074 | | |
| 38-(26)-Bc-Uterus | Biochain | A504090 | | |
| 39-(30)-Am-Placen | Ambion | 021P33A | | |
| 40-(32)-Bc-Placen | Biochain | A411073 | | |
| 41-GC-Breast | GCI | DHLR1 | | |
| 42-GC-Breast | GCI | TG6J6 | | |
| 43-GC-Breast | GCI | E6UDD | E6UDDNCF | |
| 44-(38)-Am- | Ambion | 25955 | | |
| Prostate | | | | |
| 45-Bc-Prostate | Biochain | A609258 | | |
| 46-As-Testis | Asterand | 13071 | 19567 | 19567B1 |
| 47-As-Testis | Asterand | 19671 | 42120 | 42120A1 |
| 48-GC-Artery | GCI | 7FUUP | 7FUUPAMP | |
| 49-GC-Artery | GCI | YGTVY | YGTVYAIN | |
| 50-Th-Blood- | Tel- | 52497 | | |
| PBMC | Hashomer | | | |
| 51-Th-Blood- | Tel- | 31055 | | |
| PBMC | Hashomer | | | |
| 52-Th-Blood- | Tel- | 31058 | | |
| PBMC | Hashomer | | | |
| 53-(54)-Ic-Spleen | Ichilov | CG-267 | | |
| 54-(55)-Ic-Spleen | Ichilov | 111P0106B | | |
| 55-(57)-Ic-Thymus | Ichilov | CG-98-7 | | |
| 56-(58)-Am- | Ambion | 101P0101A | | |
| Thymus | | | | |
| 57-(60)-Bc-Thyroid | Biochain | A610287 | | |
| 58-(62)-Ic-Thyroid | Ichilov | CG-119-2 | | |
| 59-Ge-Sali gland | GCI | NNSMV | NNSMVNJC | |
| 60-(67)-Ic- | Ichilov | CG-183-5 | | |
| Cerebellum | | | | |
| 61-(68)-Ic- | Ichilov | CG-212-5 | | |
| Cerebellum | | | | |
| 62-(69)-Bc-Brain | Biochain | A411322 | | |
| 63-(71)-Bc-Brain | Biochain | A411079 | | |
| 64-(72)-Ic-Brain | Ichilov | CG-151-1 | | |
| 65-(44)-Bc-Heart | Biochain | A411077 | | |
| 66-(46)-Ic-Heart | Ichilov | CG-227-1 | | |
| 67-(45)-Ic-Heart | Ichilov | CG-255-9 | | |
| (Fibrotic) | | | | |
| 68-GC-Skel Mus | GCI | T8YZS | T8YZSN7O | |
| 69-GC-Skel Mus | GCI | Q3WKA | Q3WKANCJ | |
| 70-As-Skel Mus | Asterand | 8774 | 8235 | 8235G1 |
| 71-As-Skel Mus | Asterand | 8775 | 8244 | 8244A1 |
| 72-As-Skel Mus | Asterand | 10937 | 12648 | 12648C1 |
| 73-As-Skel Mus | Asterand | 6692 | 6166 | 6166A1 |

### Materials and Experimental Procedures

RNA preparation—RNA was obtained from Clontech (Franklin Lakes, N.J. USA 07417, www.clontech.com), BioChain Inst. Inc. (Hayward, Calif. 94545 USA www.biochain.com), ABS (Wilmington, Del. 19801, USA, www.absbioreagents.com), Ambion (Austin, Tex. 78744 USA, www.ambion.com), or GOG for ovary samples—Pediatic Cooperative Human Tissue Network, Gynecologic Oncology Group Tissue Bank, Children Hospital of Columbus (Columbus Ohio 43205 USA). Alternatively, RNA was generated from tissue samples using TRI-Reagent (Molecular Research Center), according to Manufacturer's instructions. Tissue and RNA samples were obtained from patients or from postmortem. Total RNA samples were treated with DNaseI (Ambion).

RT PCR—Purified RNA (1 μg) was mixed with 150 ng Random Hexamer primers (Invitrogen) and 500 μM dNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 min at 25° C., followed by further incubation at 42° C. for 2 min. Then, 1 μl (200units) of SuperscriptII (Invitrogen) was added and the reaction (final volume of 25 μl) was incubated for 50 min at 42° C. and then inactivated at 70° C. for 15 min. The resulting cDNA was diluted 1:20 in TE buffer (10 mM Tris pH=8, 1 mM EDTA pH=8).

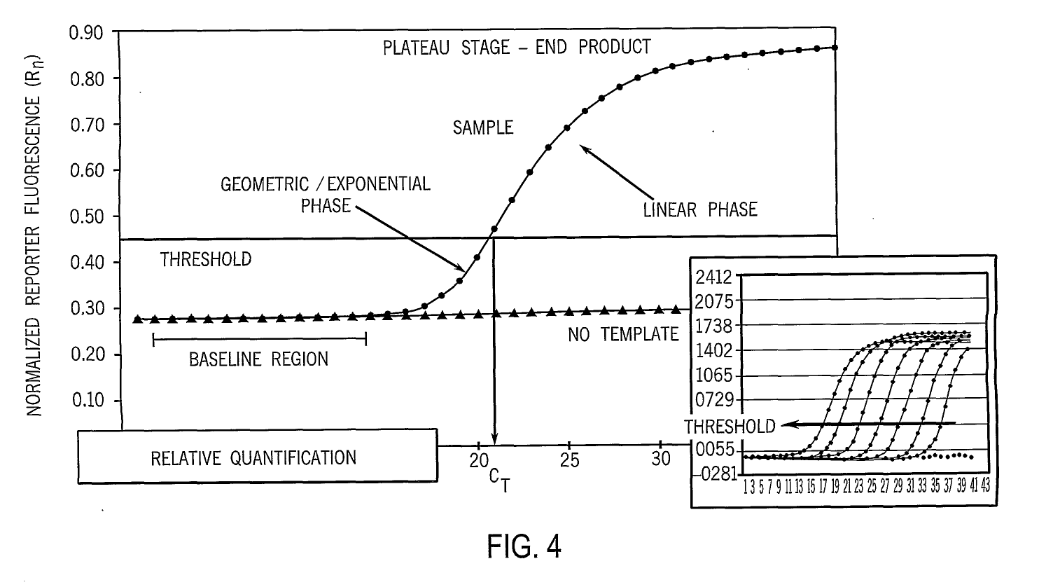

Real-Time RT-PCR analysis—cDNA (5 μl), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. The relative quantity was calculated using the equation Q=efficiency^{̂-Ct}. The efficiency of the PCR reaction was calculated from a standard curve, created by using serial dilutions of several reverse transcription (RT) reactions. To minimize inherent differences in the RT reaction, the resulting relative quantities were normalized to the geometric mean of the relative quantities of several housekeeping (HSKP) genes. Schematic summary of quantitative real-time PCR analysis is presented in FIG. 4. As shown, the x-axis shows the cycle number. The C_{T}=Threshold Cycle point, which is the cycle that the amplification curve crosses the fluorescence threshold that was set in the experiment. This point is a calculated cycle number in which PCR product signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

The sequences of the housekeeping genes measured in all the examples on ovarian cancer panel were as follows:

SDHA (GenBank Accession No. NM_004168

(SEQ ID NO: 33)

SDHA Forward primer (SEQ ID NO: 34):
TGGGAACAAGAGGGCATCTG
SDHA Reverse primer (SEQ ID NO: 35):
CCACCACTGCATCAAATTCATG
SDHA-amplicon (SEQ ID NO: 36):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323,

(SEQ ID NO: 1)

PBGD Forward primer (SEQ ID NO: 2):
TGAGAGTGATTCGCGTGGG
PBGD Reverse primer (SEQ ID NO: 3):
CCAGGGTACGAGGCTTTCAAT
PBGD-amplicon (SEQ ID NO: 4)
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194

(SEQ ID NO: 5)

HPRT1 Forward primer (SEQ ID NO: 6):
TGACACTGGCAAAACAATGCA
HPRT1 Reverse primer (SEQ ID NO: 7):
GGTCCTTTTCACCAGCAAGCT
HPRT1-amplicon (SEQ ID NO: 8):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
GAPDH (GenBank Accession No. BC026907

(SEQ ID NO: 9)

GAPDH Forward primer (SEQ ID NO: 10):
TGCACCACCAACTGCTTAGC
GAPDH Reverse primer (SEQ ID NO: 11):
CCATCACGCCACAGTTTCC
GAPDH-amplicon (SEQ ID NO: 12):
TGCACCACCAACTGCTTAGCACCCCTGGCCAAGGTCATCCATGACAACTT
TGGTATCGTGGAAGGACTCATGACCACAGTCCATGCCATCACTGCCACCC
AGAAGACTGTGGATGG

The sequences of the housekeeping genes measured in all the examples on colon cancer tissue testing panel were as follows:

PBGD (GenBank Accession No. BC019323,

(SEQ ID NO: 1)

PBGD Forward primer (SEQ ID NO: 2):
TGAGAGTGATTCGCGTGGG
PBGD Reverse primer (SEQ ID NO: 3):
CCAGGGTACGAGGCTTTCAAT
PBGD-amplicon (SEQ ID NO: 4):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194,

(SEQ ID NO: 5)

HPRT1 Forward primer (SEQ ID NO: 6):
TGACACTGGCAAAACAATGCA
HPRT1 Reverse primer (SEQ ID NO: 7):
GGTCCTTTTCACCAGCAAGCT
HPRT1-amplicon (SEQ ID NO: 8):
TGACACTGGCAAAACAATGCAGACTTTGCTTTCCTTGGTCAGGCAGTATA
ATCCAAAGATGGTCAAGGTCGCAAGCTTGCTGGTGAAAAGGACC
G6PD (GenBank Accession No. NM_000402

(SEQ ID NO: 13)

G6PD Forward primer (SEQ ID NO: 14):
gaggccgtcaccaagaacat
G6PD Reverse primer (SEQ ID NO: 15):
ggacagccggtcagagctc
G6PD-amplicon (SEQ ID NO: 16):
gaggccgtcaccaagaacatteaegagtcctgcatgagccagataggctg
gaaccgcatcatcgtggagaagcccttcgggagggacctgcagagctctg
accggctgtcc
RPS27A (GenBank Accession No. NM_002954

(SEQ ID NO: 17)

RPS27A Forward primer (SEQ ID NO: 18):
CTGGCAAGCAGCTGGAAGAT
RPS27A Reverse primer (SEQ ID NO: 19):
TTTCTTAGCACCACCACGAAGTC
RPS27A-amplicon (SEQ ID NO: 20):
CTGGCAAGCAGCTGGAAGATGGACGTACTTTGTCTGACTACAATATTCAA
AAGGAGTCTACTCTTCATCTTGTGTTGAGACTTCGTGGTGGTGCTAAGAA
A

The sequences of the housekeeping genes measured in all the examples in testing panel were as follows:

Ubiquitin (GenBank Accession No. BC000449

(SEQ ID NO: 29)

Ubiquitin Forward primer (SEQ ID NO: 30):
ATTTGGGTCGCGGTTCTTG
Ubiquitin Reverse primer (SEQ ID NO: 31):
TGCCTTGACATTCTCGATGGT
Ubiquitin-amplicon

(SEQ ID NO: 32)

ATTTGGGTCGCGGTTCTTGTTTGTGGATCGCTGTGATCGTCACTTGACAA
TGCAGATCTTCGTGAAGACTCTGACTGGTAAGACCATCACCCTCGAGGTT
GAGCCCAGTGACACCATCGAGAATGTCAAGGCA
SDHA (GenBank Accession No. NM_004168

(SEQ ID NO: 33)

SDHA Forward primer (SEQ ID NO: 34):
TGGGAACAAGAGGGCATCTG
SDHA Reverse primer (SEQ ID NO: 35):
CCACCACTGCATCAAATTCATG
SDHA-amplicon (SEQ ID NO: 36):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323,

(SEQ ID NO: 1)

PBGD Forward primer (SEQ ID NO: 2):
TGAGAGTGATTCGCGTGGG
PBGD Reverse primer (SEQ ID NO: 3):
CCAGGGTACGAGGCTTTCAAT
PBGD-amplicon (SEQ ID NO: 4):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194,

(SEQ ID NO: 5)

HPRT1 Forward primer (SEQ ID NO: 6):
TGACACTGGCAAAACAATGCA
HPRT1 Reverse primer (SEQ ID NO: 7):
GGTCCTTTTCACCAGCAAGCT
HPRT1-amplicon (SEQ ID NO: 8):
TGACACTGGCAAAACAATGCAGACTTTGCTTTCCTTGGTCAGGCAGTATA
ATCCAAAGATGGTCAAGGTCGCAAGCTTGCTGGTGAAAAGGACC

The sequences of the housekeeping genes measured in all the examples on breast cancer panel were as follows:

G6PD (Genflank Accession No. NM_000402

(SEQ ID NO: 13)

G6PD Forward primer (SEQ ID NO: 14):
gaggccgtcaccaagaacat
G6PD Reverse primer (SEQ ID NO: 15):
ggacagccggtcagagctc
G6PD-amplicon (SEQ ID NO: 16):
gaggccgtcaccaagaacattcacgagtcctgcatgagccagataggctg
gaaccgcatcatcgtggagaagcccttcgggagggacctgcagagctctg
accggctgtcc
SDHA (GenBank Accession No. NM_004168

(SEQ ID NO: 33)

SDHA Forward primer (SEQ ID NO: 34):
TGGGAACAAGAGGGCATCTG
SDHA Reverse primer (SEQ ID NO: 35):
CCACCAGTGCATCAAATTCATG
SDHA-amplicon (SEQ ID NO: 36):
TGGGAACAAGAGGGCATCTGCTAAAGTTTCAGATTCCATTTCTGCTCAGT
ATCCAGTAGTGGATCATGAATTTGATGCAGTGGTGG
PBGD (GenBank Accession No. BC019323,

(SEQ ID NO: 1)

PBGD Forward primer (SEQ ID NO: 2):
TGAGAGTGATTCGCGTGGG
PBGD Reverse primer (SEQ ID NO: 3):
CCAGGGTACGAGGCTTTCAAT
PBGD-amplicon (SEQ ID NO: 4):
TGAGAGTGATTCGCGTGGGTACCCGCAAGAGCCAGCTTGCTCGCATACAG
ACGGACAGTGTGGTGGCAACATTGAAAGCCTCGTACCCTGG
HPRT1 (GenBank Accession No. NM_000194,

(SEQ ID NO: 5)

HPRT1 Forward primer (SEQ ID NO: 6):
TGACACTGGCAAAACAATGCA
HPRT1 Reverse primer (SEQ ID NO: 7):
GGTCCTTTTCACCAGCAAGCT
HPRT1-amplicon (SEQ ID NO: 8):
TGACACTGGCAAAACAATGCAGACTTTGCTTTCCTTGGTCAGGCAGTATA
ATCCAAAGATGGTCAAGGTCGCAAGCTTGGTGGTGAAAAGGACC

The sequences of the housekeeping genes measured in all the examples on normal tissue samples panel were as follows:

RPL19 (GenBank Accession No. NM_000981

(SEQ ID NO: 21)

RPL19Forward primer (SEQ ID NO: 22):
TGGCAAGAAGAAGGTCTGGTTAG
RPL19Reverse primer (SEQ ID NO: 23):
TGATCAGCCCATCTTTGATGAG
RPL19-amplicon (SEQ ID NO: 24):
TGGCAAGAAGAAGGTCTGGTTAGACCCCAATGAGACCAATGAAATCGCCA
ATGCCAACTCCCGTCAGCAGATCCGGAAGCTCATCAAAGATGGGCTGATC
A
TATA box (GenBank Accession No. NM_003194,

(SEQ ID NO: 25)

TATA box Forward primer (SEQ ID NO: 26):
CGGTTTGCTGCGGTAATCAT
TATA box Reverse primer (SEQ ID NO: 27):
TTTCTTGCTGCCAGTCTGGAC
TATA box-amplicon (SEQ ID NO: 28):
CGGTTTGCTGCGGTAATCATGAGGATAAGAGAGCCACGAACCACGGCACT
GATTTTCAGTTCTGGGAAAATGGTGTGCACAGGAGCCAAGAGTGAAGAAC
AGTCCAGACTGGCAGCAAGAAA
Ubiquitin (GenBank Accession No. BC000449

(SEQ ID NO: 29)

Ubiquitin Forward primer (SEQ ID NO: 30):
ATTTGGGTCGCGGTTCTTG
Ubiquitin Reverse primer (SEQ ID NO: 31):
TGCCTTGACATTCTCGATGGT
Ubiquitin-amplicon

(SEQ ID NO: 32)

ATTTGGGTCGCGGTTCTTGTTTGTGGATCGCTGTGATCGTCACTTGACAA
TGCAGATCTTCGTGAAGACTCTGACTGGTAAGACCATCACCCTCGAGGTT
GAGCCCAGTGACACCATCGAGAATGTCAAGGCA
SDHA (GenBank Accession No. NM_004168

(SEQ ID NO: 33)

SDHA Forward primer (SEQ ID NO: 34):
TGGGAACAAGAGGGCATCTG
SDHA Reverse primer (SEQ ID NO: 35):
CCACCACTGCATCAAATTCATG
SDHA-amplicon (SEQ ID NO: 36):
TGGGAACAAGAGGGCATCTGCTAAAGTTTCAGATTCCATTTCTGCTCAGT
ATCCAGTAGTGGATCATGAATTTGATGCAGTGGTGG

### Actual Marker Examples

The following examples relate to specific actual marker examples.

### Description for Cluster AA340453

Cluster AA340453 features 6 transcript(s) and 46 segment(s) of interest, the names for which are given in Tables 2 and 3, respectively. The selected protein variants are given in table 4.

TABLE 2
Transcripts of interest
Transcript Name

AA340453_T3 (SEQ ID NO: 37)
AA340453_T7 (SEQ ID NO: 38)
AA340453_T8 (SEQ ID NO: 39)
AA340453_T9 (SEQ ID NO: 40)
AA340453_T11 (SEQ ID NO: 41)
AA340453_T17 (SEQ ID NO: 42)

TABLE 3
Segments of interest
Segment Name

AA340453_N9 (SEQ ID NO: 43)
AA340453_N17 (SEQ ID NO: 44)
AA340453_N21 (SEQ ID NO: 45)
AA340453_N23 (SEQ ID NO: 46)
AA340453_N24 (SEQ ID NO: 47)
AA340453_N25 (SEQ ID NO: 48)
AA340453_N27 (SEQ ID NO: 49)
AA340453_N32 (SEQ ID NO: 50)
AA340453_N44 (SEQ ID NO: 51)
AA340453_N45 (SEQ ID NO: 52)
AA340453_N61 (SEQ ID NO: 53)
AA340453_N62 (SEQ ID NO: 54)
AA340453_N0 (SEQ ID NO: 55)
AA340453_N4 (SEQ ID NO: 56)
AA340453_N6 (SEQ ID NO: 57)
AA340453_N11 (SEQ ID NO: 58)
AA340453_N12 (SEQ ID NO: 59)
AA340453_N13 (SEQ ID NO: 60)
AA340453_N15 (SEQ ID NO: 61)
AA340453_N16 (SEQ ID NO: 62)
AA340453_N19 (SEQ ID NO: 63)
AA340453_N20 (SEQ ID NO: 64)
AA340453_N29 (SEQ ID NO: 65)
AA340453_N30 (SEQ ID NO: 66)
AA340453_N34 (SEQ ID NO: 67)
AA340453_N35 (SEQ ID NO: 68)
AA340453_N36 (SEQ ID NO: 69)
AA340453_N37 (SEQ ID NO: 70)
AA340453_N38 (SEQ ID NO: 71)
AA340453_N39 (SEQ ID NO: 72)
AA340453_N42 (SEQ ID NO: 73)
AA340453_N43 (SEQ ID NO: 74)
AA340453_N46 (SEQ ID NO: 75)
AA340453_N47 (SEQ ID NO: 76)
AA340453_N48 (SEQ ID NO: 77)
AA340453_N49 (SEQ ID NO: 78)
AA340453_N50 (SEQ ID NO: 79)
AA340453_N51 (SEQ ID NO: 80)
AA340453_N52 (SEQ ID NO: 81)
AA340453_N54 (SEQ ID NO: 82)
AA340453_N55 (SEQ ID NO: 83)
AA340453_N58 (SEQ ID NO: 84)
AA340453_N63 (SEQ ID NO: 85)
AA340453_N64 (SEQ ID NO: 86)
AA340453_N65 (SEQ ID NO: 87)
AA340453_N66 (SEQ ID NO: 88)

TABLE 4
Proteins of interest

| Protein Name | Corresponding Transcript(s) |
|---|---|
| AA340453_P27 (SEQ ID NO: 91) | AA340453_T3 (SEQ ID NO: 37) |
| AA340453_P29 (SEQ ID NO: 92) | AA340453_T17 (SEQ ID NO: 42) |
| AA340453_P30 (SEQ ID NO: 93) | AA340453_T7 (SEQ ID NO: 38) |
| AA340453_P31 (SEQ ID NO: 94) | AA340453_T8 (SEQ ID NO: 39) |
| AA340453_P32 (SEQ ID NO: 95) | AA340453_T9 (SEQ ID NO: 40) |
| AA340453_P34 (SEQ ID NO: 96) | AA340453_T11 (SEQ ID NO: 41) |

These sequences are variants of the known protein Cadherin-16 precursor (SwissProt accession identifier CADG_HUMAN (SEQ ID NO:89); known also according to the synonyms Kidney-specific cadherin; Ksp-cadherin), referred to herein as the previously known protein.

Protein Cadherin-16 precursor (SEQ ID NO:89) is known or believed to have the following function(s): Cadherins are calcium dependent cell adhesion proteins. They preferentially interact with themselves in a homophilic manner in connecting cells; cadherins may thus contribute to the sorting of heterogeneous cell types. Protein Cadherin-16 precursor localization is believed to be Type I membrane protein (Probable).

The following GO Annotation(s) apply to the previously known protein. The following annotation(s) were found: cell adhesion, which are annotation(s) related to Biological Process.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of AA340453) may optionally have one or more of the following utilities, as described with regard to Table 5. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 5
Table of Utilities for Variants of AA340453, related to Cadherin-16:

| Utility | Reason | Reference |
|---|---|---|
| under expression in the | | 15886705 |
| detection of renal cell | | |
| carcinoma | | |
| differential diagnosis of | | 15712178 |
| chromophobe renal cell | | |
| carcinoma from renal | | |
| oncocytoma | | |

According to other optional embodiments of the present invention, variants of this cluster according to the present invention (amino acid and/or nucleic acid sequences of AA340453) may optionally have one or more of the following utilities, some of which are related to utilities described above. It should be noted that these utilities are optionally and preferably suitable for human and non-human animals as subjects, except where otherwise noted.

Table 6 below describes diagnostic utilities for the cluster AA340453 that were found through microarrays, including the statistical significance thereof and a reference. One or more AA340453 variants according to the present invention may optionally have one or more of these utilities.

TABLE 6

| Utility | Microarray source |
|---|---|
| 1. identification of kidney | 1. jp_atlas, GNF1, GNF2, |
| cellular damage, due to very | med_all_avg (internal |
| high expression in kidney | database). |
| 2. identification of thyroid | 2. jp_atlas, GNF1, GNF2, |
| cellular damage, due to very | med_all_avg (internal |
| high expression in thyroid | database) |
| (second to kidney). | |

As noted above, cluster AA340453 features 6 transcript(s), which were listed in Table 2 above. These transcript(s) encode for protein(s) which are variant(s) of protein Cadherin-16 precursor (SEQ ID NO:89). A description of each variant protein according to the present invention is now provided.

Variant protein AA340453_P27 (SEQ ID NO:91) according to the present invention is encoded by transcript AA340453_T3 (SEQ ID NO:37). One or more alignments to one or more previously published protein sequences are given in the alignment table on the attached CD-ROM. A brief description of the relationship of the variant protein according to the present invention to each such aligned protein is as follows:

1. Comparison report between AA340453_P27 (SEQ ID NO:91) and CADG_HUMAN (SEQ ID NO:89):

A. An isolated chimeric polypeptide encoding for AA340453_P27 (SEQ ID NO:91), comprising a first amino acid sequence being at least 90% homologous to MVPAWLWLLCVSVPQALPKAQPAELSVEVPENYGGNFPLYLTKLPLPREGAEGQIVLSG DSGKATEGPFAMDPDSGFLLVTRALDREEQAEYQLQVTLEMQDGHVLWGPQPVLVHV KDENDQVPHFSQAIyRARLSRGTRPGIPFLFLEASDRDEPGTANSDLRFHILSQAPAQPSP DMFQLEPRLGALALSPKGSTSLDHALERTYQLLVQVKDMGDQASGHQATATVEVSIIES TWVSLEPIHLAENLKVLYPHHMAQVHWSGGDVHYHLESHPPGPFEVNAEGNLYVTREL DREAQAEYLLQVRAQNSHGEDYAAPLELHVLVMDENDNVPICPPRDPTVSIPELSPPGTE VTRLSAEDADAPGSPNSHVVYQLLSPEPEDGVEGRAFQVDPTSGSVTLGVLPLRAGQNIL LLVLAMDLAGAEGGFSSTCEVEVAVTDINDHAPEFITSQIGPISLPEDVEPGTLVAMLTAI DADLEPAFRLMDFAIERGDTEGTFGLDWEPDSGHVRLRLCKNLSYEAAPSHEVVVVVQS VAKLVGPGPGPGATATVTVLVERVMPPPKLDQESYEASVPISAPAGSFLLTIQPSDPISRTL RFSLVNDSEGWLCIEKFSGEVHTAQSLQGAQPGDTYTVLVEAQDT corresponding to amino acids 1-641 of CADG_HUMAN (SEQ ID NO:89), which also corresponds to amino acids 1-641 of AA340453_P27 (SEQ ID NO:91), and a second amino acid sequence being at least 70%, optionally at least 80%, preferably at least 85%, more preferably at least 90% and most preferably at least 95% homologous to a polypeptide having the sequence GMARQ (SEQ ID NO:372) corresponding to amino acids 642-646 of AA340453_P27 (SEQ ID NO:91), wherein said first amino acid sequence and second amino acid sequence are contiguous and in a sequential order.

B. An isolated polypeptide encoding for an edge portion of AA340453_P27 (SEQ ID NO:91), comprising an amino acid sequence being at least 70%, optionally at least about 80%, preferably at least about 85%, more preferably at least about 90% and most preferably at least about 95% homologous to the sequence GMARQ (SEQ ID NO:372) of AA340453_P27 (SEQ ID NO:91).

The localization of the variant protein was determined according to results from a number of different software programs and analyses, including analyses from SignalP and other specialized programs. The variant protein is believed to be secreted.

Variant protein AA340453 P27 (SEQ ID NO:91) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein AA340453_P27 (SEQ ID NO:91) sequence provides support for the deduced sequence of this variant protein according to the present invention).

TABLE 7
Amino acid mutations

| SNP position(s) on | | |
|---|---|---|
| amino acid sequence | Alternative amino acid(s) | Previously known SNP? |
| 11 | V -> A | No |
| 74 | D -> N | No |
| 82 | R -> | No |
| 118 | D -> N | No |
| 191 | L -> F | Yes |
| 257 | H -> Y | Yes |
| 403 | G -> E | No |

The glycosylation sites of variant protein AA340453_P27 (SEQ ID NO:91), as compared to the known protein Cadherin-16 precursor (SEQ ID NO:89), are described in Table 8 (given according to their position(s) on the amino acid sequence in the first column; the second column indicates whether the glycosylation site is present in the variant protein; and the last column indicates whether the position is different on the variant protein).

TABLE 8
Glycosylation site(s)

| Position(s) on known amino | Present | Position(s) |
|---|---|---|
| acid sequence | in variant protein? | on variant protein |
| 517 | Yes | 517 |
| 602 | Yes | 602 |
| 722 | No | |

Variant protein AA340453_P27 (SEQ ID NO:91) is encoded by transcript AA340453_T3 (SEQ ID NO:37), for which the coding portion starts at position 128 and ends at position 2065. The transcript also has the following SNPs as listed in Table 9 (given according to their position on the nucleotide sequence, with the alternative nucleic acid listed).

TABLE 9
Nucleic acid SNPs

| SNIP position(s) on nucleotide | |
|---|---|
| sequence | Alternative nucleic acid(s) |
| 159 | T -> C |
| 347 | G -> A |
| 372 | G -> |
| 479 | G -> A |
| 698 | C -> T |
| 896 | C -> T |
| 1335 | G -> A |
| 2270 | A -> G |

Variant protein AA340453 P29 (SEQ ID NO:92) according to the present invention is encoded by transcript AA340453_T17 (SEQ ID NO:42). One or more alignments to one or more previously published protein sequences are given in the alignment table on the attached CD-ROM. A brief description of the relationship of the variant protein according to the present invention to each such aligned protein is as follows:

1. Comparison report between AA340453_P29 (SEQ ID NO:92) and CADG_{—}1HUMAN (SEQ ID NO:89):

A. An isolated chimeric polypeptide encoding for AA340453_P29 (SEQ ID NO:92), comprising a first amino acid sequence being at least 90% homologous to MVPAWLWLLCVSVPQALPKAQPAELSVEVPENYGGNFPLYLTKLPLPREGAEGQIVLSG DSGKATEGPFAMDPDSGFLLVTRALDREEQAEYQLQVTLEMQDGHVLWGPQPVLVHV KDENDQVPHFSQAIYRARLSRGTRP corresponding to amino acids 1-141 of CADG_HUMAN (SEQ ID NO:89), which also corresponds to amino acids 1-141 of AA340453_P29 (SEQ ID NO:92), and a second amino acid sequence being at least 70%, optionally at least 80%, preferably at least 85%, more preferably at least 90% and most preferably at least 95% homologous to a polypeptide having the sequence VCPPTVNQASPSSSLRLQTGMSQAQPTRIFDSTS (SEQ ID NO:436) corresponding to amino acids 142-175 of AA340453_P29 (SEQ ID NO:92), wherein said first amino acid sequence and second amino acid sequence are contiguous and in a sequential order.

B. An isolated polypeptide encoding for an edge portion of AA340453_P29 (SEQ ID NO:92), comprising an amino acid sequence being at least 70%, optionally at least about 80%, preferably at least about 85%, more preferably at least about 90% and most preferably at least about 95% homologous to the sequence VCPPTVNQASPSSSLRLQTGMSQAQPTRIFDSTS (SEQ ID NO:436) of AA340453_P29 (SEQ ID NO:92).

2. Comparison report between AA340453_P29 (SEQ ID NO:92) and Q6UW93_HUMAN (SEQ ID NO:90):

A. An isolated chimeric polypeptide encoding for AA340453_P29 (SEQ ID NO:92), comprising a first amino acid sequence being at least 90% homologous to MVPAWLWLLCVSVPQALPKAQPAELSVEVPENYGGNFPLYLTKLPLPREGAEGQIVLSG DSGKATEGPFAMDPDSGFLLVTRALDREEQAEYQLQVTLEMQDGHVLWGPQPVLVHV KDENDQVPHFSQAIYRARLSRGTRP corresponding to amino, acids 1-141 of Q6UW93_HUMAN (SEQ ID NO:90), which also corresponds to amino acids 1-141 of AA340453_P29 (SEQ ID NO:92), and a second amino acid sequence being at least 70%, optionally at least 80%, preferably at least 85%, more preferably at least 90% and most preferably at least 95% homologous to a polypeptide having the sequence VCPPTVNQASPSSSLRLQTGMSQAQPTRIFDSTS (SEQ ID NO:436) corresponding to amino acids 142-175 of AA340453_P29 (SEQ ID NO:92), wherein said first amino acid sequence and second amino acid sequence are contiguous and in a sequential order.

B. An isolated polypeptide encoding for an edge portion of AA340453_P29 (SEQ ID NO:92), comprising an amino acid sequence being at least 70%, optionally at least about 80%, preferably at least about 85%, more preferably at least about 90% and most preferably at least about 95% homologous to the sequence VCPPTVNQASPSSSLRLQTGMSQAQPTRIFDSTS (SEQ ID NO:436) of AA340453_P29 (SEQ ID NO:92).

The localization of the variant protein was determined according to results from a number of different software programs and analyses, including analyses from SignalP and other specialized programs. The variant protein is believed to be secreted.

Variant protein AA340453_P29 (SEQ ID NO:92) also has the following non-silent SNPs (Single Nucleotide Polymorphisms) as listed in Table 10, (given according to their position(s) on the amino acid sequence, with the alternative amino acid(s) listed).

TABLE 10
Amino acid mutations

| SNP position(s) on amino acid | |
|---|---|
| sequence | Alternative amino acid(s) |
| 11 | V -> A |
| 74 | D -> N |
| 82 | R -> |
| 118 | D -> N |

The glycosylation sites of variant protein AA340453_P29 (SEQ ID NO:92), as compared to the known protein Cadherin-16 precursor (SEQ ID NO:89), are described in Table 11 (given according to their position(s) on the amino acid sequence in the first column; the second column indicates whether the glycosylation site is present in the variant protein; and the last column indicates whether the position is different on the variant protein).

TABLE 11
Glycosylation site(s)

| Position(s) on known amino | |
|---|---|
| acid sequence | Present in variant protein? |
| 517 | No |
| 602 | No |
| 722 | No |

Variant protein AA340453_P29 (SEQ ID NO:92) is encoded by transcript AA340453_T17 (SEQ ID NO:42), for which the coding portion starts at position 128 and ends at position 652. The transcript also has the following SNPs as listed in Table 12 (given according to their position on the nucleotide sequence, with the alternative nucleic acid listed.).

TABLE 12
Nucleic acid SNPs

| SNP position(s) on nucleotide | |
|---|---|
| sequence | Alternative nucleic acid(s) |
| 159 | T -> C |
| 347 | G -> A |
| 372 | G -> |
| 479 | G -> A |
| 721 | C -> T |
| 909 | C -> T |
| 1348 | G -> A |
| 2283 | A -> G |

Variant protein AA340453_P30 (SEQ ID NO:93) according to the present invention is encoded by transcript AA340453_T7 (SEQ ID NO:38). One or more alignments to one or more previously published protein sequences are given in the alignment table on the attached CD-ROM. A brief description of the relationship of the variant protein according to the present invention to each such aligned protein is as follows:

1. Comparison report between AA340453 P30 (SEQ ID NO:93) and CADG_Human (SEQ ID NO:89):

A. An isolated chimeric polypeptide encoding for AA340453_P30 (SEQ ID NO:93), comprising a first amino acid sequence being at least 90% homologous to MVPAWLWLLCVSVPQALPKAQPAELSVEVPENYGGNFPLYLTKLPLPREGAEGQIVLSG DSGKATEGPFAMDPDSGFLLVTRALDREEQAEYQLQVTLEMQDGHVLWGPQPVLVHV KDENDQVPHFSQAIYRARLSRGTRPGIPFLFLEASDRDEPGTANSDLRFHILSQAPAQPSP DMFQLEPRLGALALSPKGSTSLDHALERTYQLLVQVKDMGDQASGHQATATVEVSIIES TWVSLEPIHLAENLKVLYPHHMAQVHWSGGDVHYHLESHPPGPFEVNAEGNLYVTREL DREAQAE corresponding to amino acids 1-301 of CADG_HUMAN (SEQ ID NO:89), which also corresponds to amino acids 1-301 of AA340453_P30 (SEQ ID NO:93), and a second amino acid sequence being having the sequence VI corresponding to amino acids 302-303 of AA340453_P30 (SEQ ID NO:93), wherein said first amino acid sequence and second amino acid sequence are contiguous and in a sequential order.

The localization of the variant protein was determined according to results from a number of different software programs and analyses, including analyses from SignalP and other specialized programs. The variant protein is believed to be secreted.

Variant protein AA340453_P30 (SEQ ID NO:93) also has the following non-silent SNPs (Single Nucleotide Polymorphisms) as listed in Table 13, (given according to their position(s) on the amino acid sequence, with the alternative amino acid(s) listed).

TABLE 13
Amino acid mutations

| SNP position(s) on amino acid | |
|---|---|
| sequence | Alternative amino acid(s) |
| 11 | V -> A |
| 74 | D -> N |
| 82 | R -> |
| 118 | D -> N |
| 191 | L -> F |
| 257 | H -> Y |

The glycosylation sites of variant protein AA340453_P30 (SEQ ID NO:93), as compared to the known protein Cadherin-16 precursor (SEQ ID NO:89), are described in Table 14 (given according to their position(s) on the amino acid sequence in the first column; the second column indicates whether the glycosylation site is present in the variant protein; and the last column indicates whether the position is different on the variant protein).

TABLE 14
Glycosylation site(s)

| Position(s) on known amino | |
|---|---|
| acid sequence | Present in variant protein? |
| 517 | No |
| 602 | No |
| 722 | No |

Variant protein AA340453_P30 (SEQ ID NO:93) is encoded by the transcript AA340453_T7 (SEQ ID NO:38), for which the coding portion starts at position 128 and ends at position 1036. The transcript also has the following SNPs as listed in Table 15 (given according to their position on the nucleotide sequence, with the alternative nucleic acid listed).

TABLE 15
Nucleic acid SNPs

| SNP position(s) on nucleotide | |
|---|---|
| sequence | Alternative nucleic acid(s) |
| 159 | T -> C |
| 347 | G -> A |
| 372 | G -> |
| 479 | G -> A |
| 698 | C -> T |
| 896 | C -> T |
| 1041 | G -> C |
| 1061 | C -> T |
| 1544 | G -> A |

Variant protein AA340453_P31 (SEQ ID NO:94) according to the present invention is encoded by transcript AA340453_T8 (SEQ ID NO:39). One or more alignments to one or more previously published protein sequences are given in the alignment table on the attached CD-ROM. A brief description of the relationship of the variant protein according to the present invention to each such aligned protein is as follows:

1. Comparison report between AA340453_P31 (SEQ ID NO:94) and CADG_HUMAN (SEQ ID NO:89):

A. An isolated chimeric polypeptide encoding for AA340453_P31 (SEQ ID NO:94), comprising a first amino acid sequence being at least 90% homologous to MVPAWLWLLCVSVPQALPKAQPAELSVEVPENYGGNFPLYLTKLPLPREGAEGQIVLSG DSGKATEGPFAMDPDSGFLLVTRALDREEQAEYQLQVTLEMQDGHVLWGPQPVLVHV KDENDQVPHFSQAIYRARLSRGTRPGIPFLFLEASDRDEPGTANSDLRFHILSQAPAQPSP DMFQLEPRLGALALSPK corresponding to amino acids 1-194 of CADG_HUMAN (SEQ ID NO:89), which also corresponds to amino acids 1-194 of AA340453_P31 (SEQ ID NO:94), and a second amino acid sequence being at least 70%, optionally at least 80%, preferably at least 85%, more preferably at least 90% and most preferably at least 95% homologous to a polypeptide having the sequence ALTTPWRGPTSCWYRSRTWVTRPQATRPLPPWKSPS (SEQ ID NO:373) corresponding to amino acids 195-230 of AA340453_P31 (SEQ ID NO:94), wherein said first amino acid sequence and second amino acid sequence are contiguous and in a sequential order.

B. An isolated polypeptide encoding for an edge portion of AA340453_P31 (SEQ ID NO:94), comprising an amino acid sequence being at least 70%, optionally at least about 80%, preferably at least about 85%, more preferably at least about 90% and most preferably at least about 95% homologous to the sequence ALTTPWRGPTSCWYRSRTWVTRPQATRPLPPWKSPS (SEQ ID NO:373) of AA340453_P31 (SEQ ID NO:94).

The localization of the variant protein was determined according to results from a number of different software programs and analyses, including analyses from SignalP and other specialized programs. The variant protein is believed to be secreted.

Variant protein AA340453_P31 (SEQ ID NO:94) also has the following non-silent SNPs (Single Nucleotide Polymorphisms) as listed in Table 16, (given according to their position(s) on the amino acid sequence, with the alternative amino acid(s) listed).

TABLE 16
Amino acid mutations

| SNP position(s) on amino acid | |
|---|---|
| sequence | Alternative amino acid(s) |
| 11 | V -> A |
| 74 | D -> N |
| 82 | R -> |
| 118 | D -> N |
| 191 | L -> F |

The glycosylation sites of variant protein AA340453_P31 (SEQ ID NO:94), as compared to the known protein Cadherin-16 precursor (SEQ ID NO:89), are described in Table 17 (given according to their position(s) on the amino acid sequence in the first column; the second column indicates whether the glycosylation site is present in the variant protein; and the last column indicates whether the position is different on the variant protein).

TABLE 17
Glycosylation site(s)

| Position(s) on known amino | |
|---|---|
| acid sequence | Present in variant protein? |
| 517 | No |
| 602 | No |
| 722 | No |

Variant protein AA340453_P31 (SEQ ID NO:94) is encoded by the transcript AA340453_T8 (SEQ ID NO:39), for which the coding portion starts at position 128 and ends at position 817. The transcript also has the following SNPs as listed in Table 18 (given according to their position on the nucleotide sequence, with the alternative nucleic acid listed

TABLE 18
Nucleic acid SNPs

| SNP position(s) on nucleotide | |
|---|---|
| sequence | Alternative nucleic acid(s) |
| 159 | T -> C |
| 347 | G -> A |
| 372 | G -> |
| 479 | G -> A |
| 698 | C -> T |
| 886 | C -> T |
| 1325 | G -> A |

Variant protein AA340453_P32 (SEQ ID NO:95) according to the present invention is encoded by transcript AA340453_T9 (SEQ ID NO:40). One or more alignments to one or more previously published protein sequences are given in the alignment table on the attached CD-ROM. A brief description of the relationship of the variant protein according to the present invention to each such aligned protein is as follows:

1. Comparison report between AA340453_P32 (SEQ ID NO:95) and CADG_HUMAN (SEQ ID NO:89):

A. An isolated chimeric polypeptide encoding for AA340453_P32 (SEQ ID NO:95), comprising a first amino acid sequence being at least 90% homologous to MVPAWLWLLCVSVPQALPKAQPAELSVEVPENYGGNFPLYLTKLPLPREGAEGQIVLSG DSGKATEGPFAMDPDSGFLLVTRALDREEQAEYQLQVTLEMQDGHVLWGPQPVLVHV KDENDQVPHFSQAIYRARLSRGTRPG corresponding to amino acids 1-142 of CADG_HUMAN (SEQ ID NO:89), which also corresponds to amino acids 1-142 of AA340453_P32 (SEQ ID NO:95), and a second amino acid sequence being at least 70%, optionally at least 80%, preferably at least 85%, more preferably at least 90% and most preferably at least 95% homologous to a polypeptide having the sequence FRPG (SEQ ID NO:374) corresponding to amino acids 143-146 of AA340453_P32 (SEQ ID NO:95), wherein said first amino acid sequence and second amino acid sequence are contiguous and in a sequential order.

B. An isolated polypeptide encoding for an edge portion of AA340453_P32 (SEQ ID NO:95), comprising an amino acid sequence being at least 70%, optionally at least about 80%, preferably at least about 85%, more preferably at least about 90% and most preferably at least about 95% homologous to the sequence FRPG (SEQ ID NO:374) of AA340453_P32 (SEQ ID NO:95).

The localization of the variant protein was determined according to results from a number of different software programs and analyses, including analyses from SignalP and other specialized programs. The variant protein is believed to be secreted.

Variant protein AA340453_P32 (SEQ ID NO:95) also has the following non-silent SNPs (Single Nucleotide Polymorphisms) as listed in Table 19, (given according to their position(s) on the amino acid sequence, with the alternative amino acid(s) listed).

TABLE 19
Amino acid mutations

| SNP position(s) on amino acid | |
|---|---|
| sequence | Alternative amino acid(s) |
| 11 | V -> A |
| 74 | D -> N |
| 82 | R -> |
| 118 | D -> N |

The glycosylation sites of variant protein AA340453_P32 (SEQ ID NO:95), as compared to the known protein Cadherin-16 precursor (SEQ ID NO:89), are described in Table 20 (given according to their position(s) on the amino acid sequence in the first column; the second column indicates whether the glycosylation site is present in the variant protein; and the last column indicates whether the position is different on the variant protein).

TABLE 20
Glycosylation site(s)

| Position(s) on known amino | |
|---|---|
| acid sequence | Present in variant protein? |
| 517 | No |
| 602 | No |
| 722 | No |

Variant protein AA340453_P32 (SEQ ID NO:95) is encoded by the transcript AA340453_T9 (SEQ ID NO:40), for which the coding portion starts at position 128 and ends at position 565. The transcript also has the following SNPs as listed in Table 21 (given according to their position on the nucleotide sequence, with the alternative nucleic acid listed).

TABLE 21
Nucleic acid SNPs

| SNP position(s) on nucleotide | |
|---|---|
| sequence | Alternative nucleic acid(s) |
| 159 | T -> C |
| 347 | G -> A |
| 372 | G -> |
| 479 | G -> A |
| 675 | C -> T |
| 873 | C -> T |
| 1312 | G -> A |

Variant protein AA340453_P34 (SEQ ID NO:96) according to the present invention is encoded by transcript AA340453_T11 (SEQ ID NO:41). One or more alignments to one or more previously published protein sequences are given in the alignment table on the attached CD-ROM. A brief description of the relationship of the variant protein according to the present invention to each such aligned protein is as follows:

1. Comparison report between AA340453_P34 (SEQ ID NO:96) and CADG_HUMAN (SEQ ID NO:89):

A. An isolated chimeric polypeptide encoding for AA340453_P34 (SEQ ID NO:96), comprising a first amino acid sequence being at least 90% homologous to MVPAWLWLLCVSVPQALPKAQPAELSVEVPENYGGNFPLYLTKLPLPREGAEGQIVLSG DSGKATEGPFAMDPDSGFLLVTRALDREEQAEYQLQVTLEMQDGHVLWGPQPVLVHV KDENDQVPHFSQAIYRARLSRGTRPGIPFLFLEASDRDEPGTANSDLRFHILSQAPAQPSP DMFQLEPRLGALALSPKGSTSLDHALERTYQLLVQVKDMGDQASGHQATATVEVSIIES TWVSLEPIHLAENLKVLYPHHMAQVHWSGGDVHYHLESHPPGPFEVNAEGNLYVTREL DREAQAEYLLQVRAQNSHGEDYAAPLELHVLVMDENDNVPICPPRDPTVSIPELSPPGTE VTRLSAEDADAPGSPNSHVVYQLLSPEPEDGVEGRAFQVDPTSGSVTLGVLPLRAGQNIL LLVLAMDLAGAEGGFSSTCEVEVAVTDINDHAPEFITSQIGPISLPEDVEPGTLVAMLTAI DADLEPAFRLMDFAIERGDTEGTFGLDWEPDSGHVRLRLCKNLSYEAAPSHEVVVVVQS VAKLVGPGPGPGATATVTVLVERVMPPPKLDQESYEASVPISAPAGSFLLTIQPSDPISRTL RFSLVNDSEGWLCIEKFSGEVHTAQSLQGAQPGDTYTVLVEAQDTDEPRLSASAPLVIHF LKAPPAPALTLAPVPSQYLCTPRQDHGLIVSGPSKDPDLASGHGPYSFTLGPNPTVQRDW RLQTLNGSHAYLTLALHWVEPREMIPVVVSHNAQMWQLLVRVIVCRCNVEGQCMRKV GRMKGMPTKSAVGILVGTLVAI corresponding to amino acids 1-797 of CADG_HUMAN (SEQ ID NO:89), which also corresponds to amino acids 1-797 of AA340453_P34 (SEQ ID NO:96), and a second amino acid sequence being at least 70%, optionally at least 80%, preferably at least 85%, more preferably at least 90% and most preferably at least 95% homologous to a polypeptide having the sequence AHCCDEPFSWRSMELGSNLLVPRCPQEKGTLEAEYTGSASEG (SEQ ID NO:375) corresponding to amino acids 798-839 of AA340453_P34 (SEQ ID NO:96), wherein said first amino acid sequence and second amino acid sequence are contiguous and in a sequential order.

B. An isolated polypeptide encoding for an edge portion of AA340453_P34 (SEQ ID NO:96), comprising an amino acid sequence being at least 70%, optionally at least about 80%, preferably at least about 85%, more preferably at least about 90% and most preferably at least about 95% homologous to the sequence AHCCDEPFSWRSMELGSNLLVPRCPQEKGTLEAEYTGSASEG (SEQ ID NO:375) of AA340453_P34 (SEQ ID NO:96).3. Comparison report between AA340453_P34 (SEQ ID NO:96) and Q6UW93_HUMAN (SEQ ID NO:90):

A. An isolated chimeric polypeptide encoding for AA340453_P34 (SEQ ID NO:96), comprising a first amino acid sequence being at least 90% homologous to MVPAWLWLLCVSVPQALPKAQPAELSVEVPENYGGNFPLYLTKLPLPREGAEGQIVLSG DSGKATEGPFAMDPDSGFLLVTRALDREEQAEYQLQVTLEMQDGHVLWGPQPVLVHV KDENDQVPHFSQAIYRARLSRGTRPGIPFLFLEASDRDEPGTANSDLRFHILSQAPAQPSP DMFQLEPRLGALALSPKGSTSLDHALERTYQLLVQVKDMGDQASGHQATATVEVSIIES TWVSLEPIHLAENLKVLYPHHMAQVHWSGGDVHYHLESHPPGPFEVNAEGNLYVTREL DREAQAEYLLQVRAQNSHGEDYAAPLELHVLVMDENDNVPICPPRDPTVSIPELSPPGTE VTRLSAEDADAPGSPNSHVVYQLLSPEPEDGVEGRAFQVDPTSGSVTLGVLPLRAGQNIL LLVLAMDLAGAEGGFSSTCEVEVAVTDINDHAPEFITSQIGPISLPEDVEPGTLVAMLTAI DADLEPAFRLMDFAIERGDTEGTFGLDWEPDSGHVRLRLCKNLSYEAAPSHEVVVVVQS VAKLVGPGPGPGATATVTVLVERVMPPPKLDQESYEASVPISAPAGSFLLTIQPSDPISRTL RFSLVNDSEGWLCIEKFSGEVHTAQSLQGAQPGDTYTVLVEAQDT corresponding to amino acids 1-641 of Q6UW93 HUMAN (SEQ ID NO:90), which also corresponds to amino acids 1-641 of AA340453_P34 (SEQ ID NO:96), a second amino acid sequence being at least 70%, optionally at least 80%, preferably at least 85%, more preferably at least 90% and most preferably at least 95% homologous to a polypeptide having the sequence DEPRLSASAPLVIBFLKAPPAP (SEQ ID NO:376) corresponding to amino acids 642-663 of AA340453_P34 (SEQ ID NO:96), a third amino acid sequence being at least 90% homologous to ALTLAPVPSQYLCTPRQDHGLIVSGPSKDPDLASGHGPYSFTLGPNPTVQRDWRLQTLNG SHAYLTLALHWVEPREHIIPVVVSHNAQMWQLLVRVIVCRCNVEGQCMRKVGRMKGM PTKLSAVGILVGTLVAI corresponding to amino acids 642-775 of Q6UW93_HUMAN (SEQ ID NO:90), which also corresponds to amino acids 664-797 of AA340453_P34 (SEQ ID NO:96), and a fourth amino acid sequence being at least 70%, optionally at least 80%, preferably at least 85%, more preferably at least 90% and most preferably at least 95% homologous to a polypeptide having the sequence AHCCDEPFSWRSMELGSNLLVPRCPQEKGTLEAEYTGSASEG (SEQ ID NO:375) corresponding to amino acids 798-839 of AA340453_P34 (SEQ ID NO:96), wherein said first amino acid sequence, second amino acid sequence, third amino acid sequence and fourth amino acid sequence are contiguous and in a sequential order.

B. An isolated polypeptide encoding for an edge portion of AA340453 P34 (SEQ ID NO:96), comprising an amino acid sequence being at least 70%, optionally at least about 80%, preferably at least about 85%, more preferably at least about 90% and most preferably at least about 95% homologous to the sequence DEPRLSASAPLVIHFLKAPPAP (SEQ ID NO:376) of AA340453_P34 (SEQ ID NO:96).

C. An isolated polypeptide encoding for an edge portion of AA340453_P34 (SEQ ID NO:96), comprising an amino acid sequence being at least 70%, optionally at least about 80%, preferably at least about 85%, more preferably at least about 90% and most preferably at least about 95% homologous to the sequence AHCCDEPFSWRSMELGSNLLVPRCPQEKGTLEAEYTGSASEG (SEQ ID NO:375) of AA340453 P34 (SEQ ID NO:96). The localization of the variant protein was determined according to results from a number of different software programs and analyses, including analyses from SignalP and other specialized programs. The variant protein is believed to be secreted.

Variant protein AA340453 P34 (SEQ ID NO:96) also has the following non-silent SNPs (Single Nucleotide Polymorphisms) as listed in Table 22, (given according to their position(s) on the amino acid sequence, with the alternative amino acid(s) listed).

TABLE 22
Amino acid mutations

| SNP position(s) on amino acid | |
|---|---|
| sequence | Alternative amino acid(s) |
| 11 | V -> A |
| 74 | D -> N |
| 82 | R -> |
| 118 | D -> N |
| 191 | L -> F |
| 257 | H -> Y |
| 403 | G -> E |

The glycosylation sites of variant protein AA340453_P34 (SEQ ID NO:96), as compared to the known protein Cadherin-16 precursor (SEQ ID NO:89), are described in Table 23 (given according to their position(s) on the amino acid sequence in the first column; the second column indicates whether the glycosylation site is present in the variant protein; and the last column indicates whether the position is different on the variant protein).

TABLE 23
Glycosylation site(s)

| Position(s) on known | Present in | Position(s) on |
|---|---|---|
| amino acid sequence | variant protein? | variant protein |
| 517 | Yes | 517 |
| 602 | Yes | 602 |
| 722 | Yes | 722 |

Variant protein AA340453_P34 (SEQ ID NO:96) is encoded by the transcript AA340453_T11 (SEQ ID NO:41), for which the coding portion starts at position 128 and ends at position 2644. The transcript also has the following SNPs as listed in Table 24 (given according to their position on the nucleotide sequence, with the alternative nucleic acid listed; the last column indicates whether the SNP is known or not; the presence of known SNPs in variant protein AA340453_P34 (SEQ ID NO:96) sequence provides support for the deduced sequence of this variant protein according to the present invention).

TABLE 24
Nucleic acid SNPs

| SNP position(s) on nucleotide | |
|---|---|
| sequence | Alternative nucleic acid(s) |
| 159 | T -> C |
| 347 | G -> A |
| 372 | G -> |
| 479 | G -> A |
| 698 | C -> T |
| 896 | C -> T |
| 1335 | G -> A |

As noted above, cluster AA340453 features 46 segments, which were listed in Table 3. These segments are portions of nucleic acid sequence(s), some of which are described herein separately because they are of particular interest. A description of some of the segment according to the present invention is now provided.

Segment cluster AA340453_N17 (SEQ ID NO:44) according to the present invention is supported by 33 libraries. The number of libraries was determined as previously described. This segment can be found in the following transcript(s): AA340453_T11 (SEQ ID NO:41), AA340453_T17 (SEQ ID NO:42), AA340453_T3 (SEQ ID NO:37), AA340453_T7 (SEQ ID NO:38), AA340453_T8 (SEQ ID NO:39) and AA340453_T9 (SEQ ID NO:40). Table 25 below describes the starting and ending position of this segment on each transcript.

TABLE 25
Segment location on transcripts

| | Segment | Segment |
|---|---|---|
| Transcript name | starting position | ending position |
| AA340453_T11 (SEQ ID NO: 41) | 575 | 710 |
| AA340453_T17 (SEQ ID NO: 42) | 598 | 733 |
| AA340453_T3 (SEQ ID NO: 37) | 575 | 710 |
| AA340453_T7 (SEQ ID NO: 38) | 575 | 710 |
| AA340453_T8 (SEQ ID NO: 39) | 575 | 710 |
| AA340453_T9 (SEQ ID NO: 40) | 552 | 687 |

Segment cluster AA340453_N24 (SEQ ID NO:14) according to the present invention is supported by 3 libraries. The number of libraries was determined as previously described. This segment can be found in the following transcript(s): AA340453_T7 (SEQ ID NO:38). Table 26 below describes the starting and ending position of this segment on each transcript.

TABLE 26
Segment location on transcripts

| | Segment | Segment |
|---|---|---|
| Transcript name | starting position | ending position |
| AA340453_T7 (SEQ ID NO: 38) | 1031 | 1239 |

Segment cluster AA340453_N44 (SEQ ID NO:51) according to the present invention is supported by 3 libraries. The number of libraries was determined as previously described. This segment can be found in the following transcript(s): AA340453_T17 (SEQ ID NO:42) and AA340453_T3 (SEQ ID NO:37). Table 27 below describes the starting and ending position of this segment on each transcript.

TABLE 27
Segment location on transcripts

| | Segment | Segment |
|---|---|---|
| Transcript name | starting position | ending position |
| AA340453_T17 (SEQ ID NO: 42) | 2065 | 2284 |
| AA340453_T3 (SEQ ID NO: 37) | 2052 | 2271 |

Segment cluster AA340453 N45 (SEQ ID NO:52) according to the present invention is supported by 5 libraries. The number of libraries was determined as previously described. This segment can be found in the following transcript(s): AA340453_T17 (SEQ ID NO:42) and AA340453_T3 (SEQ ID NO:37). Table 28 below describes the starting and ending position of this segment on each transcript.

TABLE 28
Segment location on transcripts

| | Segment | Segment |
|---|---|---|
| Transcript name | starting position | ending position |
| AA340453_T17 (SEQ ID NO: 42) | 2285 | 2743 |
| AA340453_T3 (SEQ ID NO: 37) | 2272 | 2730 |

Segment cluster AA340453_N61 (SEQ ID NO:53) according to the present invention is supported by 3 libraries. The number of libraries was determined as previously described. This segment can be found in the following transcript(s): AA340453_T11 (SEQ ID NO:41). Table 29 below describes the starting and ending position of this segment on each transcript.

TABLE 29
Segment location on transcripts

| | Segment | Segment |
|---|---|---|
| Transcript name | starting position | ending position |
| AA340453_T11 (SEQ ID NO: 41) | 2520 | 2835 |

According to an optional embodiment of the present invention, short segments related to the above cluster are also provided. These segments are up to about 120 bp in length, and so are included in a separate description.

Segment cluster AA340453_N13 (SEQ ID NO:60) according to the present invention is supported by 29 libraries. The number of libraries was determined as previously described. This segment can be found in the following transcript(s): AA340453_T11 (SEQ ID NO:41), AA340453_T17 (SEQ ID NO:42), AA340453_T3 (SEQ ID NO:37), AA340453_T7 (SEQ ID NO:38), AA340453_T8 (SEQ ID NO:39) and AA340453_T9 (SEQ ID NO:40). Table 30 below describes the starting and ending position of this segment on each transcript.

TABLE 30
Segment location on transcripts

| | Segment | Segment |
|---|---|---|
| Transcript name | starting position | ending position |
| AA340453_T11 (SEQ ID NO: 41) | 517 | 551 |
| AA340453_T17 (SEQ ID NO: 42) | 517 | 551 |
| AA340453_T3 (SEQ ID NO: 37) | 517 | 551 |
| AA340453_T7 (SEQ ID NO: 38) | 517 | 551 |
| AA340453_T8 (SEQ ID NO: 39) | 517 | 551 |
| AA340453_T9 (SEQ ID NO: 40) | 517 | 551 |

Segment cluster AA340453_N15 (SEQ ID NO:61) according to the present invention is supported by 4 libraries. The number of libraries was determined as previously described. This segment can be found in the following transcript(s): AA340453_T17 (SEQ ID NO:42). Table 31 below describes the starting and ending position of this segment on each transcript.

TABLE 31
Segment location on transcripts

| | Segment | Segment |
|---|---|---|
| Transcript name | starting position | ending position |
| AA340453_T17 (SEQ ID NO: 42) | 552 | 574 |

Segment cluster AA340453_N20 (SEQ ID NO:64) according to the present invention is supported by 26 libraries. The number of libraries was determined as previously described. This segment can be found in the following transcript(s): AA340453_T11 (SEQ ID NO:41), AA340453_T17 (SEQ ID NO:42), AA340453_T3 (SEQ ID NO:37), AA340453_T7 (SEQ ID NO:38), AA340453_T8 (SEQ ID NO:39) and AA340453_T9 (SEQ ID NO:40). Table 32 below describes the starting and ending position of this segment on each transcript.

TABLE 32
Segment location on transcripts

| | Segment | Segment |
|---|---|---|
| Transcript name | starting position | ending position |
| AA340453_T11 (SEQ ID NO: 41) | 721 | 784 |
| AA340453_T17 (SEQ ID NO: 42) | 734 | 797 |
| AA340453_T3 (SEQ ID NO: 37) | 721 | 784 |
| AA340453_T7 (SEQ ID NO: 38) | 721 | 784 |
| AA340453_T8 (SEQ ID NO: 39) | 711 | 774 |
| AA340453_T9 (SEQ ID NO: 40) | 698 | 761 |

Segment cluster AA340453_N55 (SEQ ID NO:83) according to the present invention is supported by 40 libraries. The number of libraries was determined as previously described. This segment can be found in the following transcript(s): AA340453_T11 (SEQ ID NO:41), AA340453_T17 (SEQ ID NO:42), AA340453_T3 (SEQ ID NO:37), AA340453_T7 (SEQ ID NO:38), AA340453_T8 (SEQ ID NO:39) and AA340453_T9 (SEQ ID NO:40). Table 33 below describes the starting and ending position of this segment on each transcript.

TABLE 33
Segment location on transcripts

| | Segment | Segment |
|---|---|---|
| Transcript name | starting position | ending position |
| AA340453_T11 (SEQ ID NO: 41) | 2300 | 2402 |
| AA340453_T17 (SEQ ID NO: 42) | 2992 | 3094 |
| AA340453_T3 (SEQ ID NO: 37) | 2979 | 3081 |
| AA340453_T7 (SEQ ID NO: 38) | 2509 | 2611 |
| AA340453_T8 (SEQ ID NO: 39) | 2290 | 2392 |
| AA340453_T9 (SEQ ID NO: 40) | 2277 | 2379 |

Expression of Homo sapiens Cadherin 16, KSP-Cadherin (CDH16) AA340453 Transcripts which are Detectable by Amplicon as Depicted in Sequence Name AA340453_seg24F2R2 (SEQ ID NO:99) Specifically in Kidney Tissue

Expression of Homo sapiens cadherin 16, KSP-cadherin (CDH16) transcripts detectable by or according to seg24F2R2—AA340453_seg24F2R2 (SEQ ID NO:99) amplicon and primers AA340453_seg24F2 (SEQ ID NO:97) and AA340453_seg24R2 (SEQ ID NO:98) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

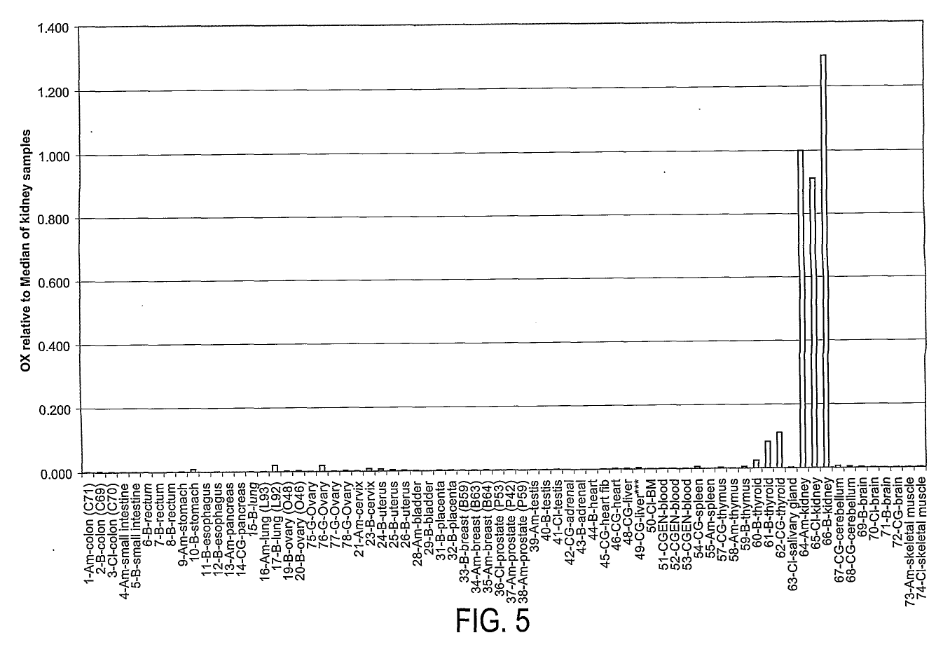

FIG. 5 is a histogram showing relative expression of the above-indicated Homo sapiens cadherin 16, KSP-cadherin (CDH16) transcripts in kidney tissue samples as opposed to other tissues.

As is evident from FIG. 5, the expression of Homo sapiens cadherin 16, KSP-cadherin (CDH16) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: AA340453_seg24F2 (SEQ ID NO:97) forward primer; and AA340453_seg4R2 (SEQ ID NO:98) reverse primer.

The present invention also preferably encompasses any amplicon obtained through the use of any suitable primer pair; for example, for the above experiment, the following amplicon was obtained as a non-limiting illustrative example only of a suitable amplicon: AA340453_seg24F2R2 (SEQ ID NO:99)

Forward Primer (AA340453_seg24F2 (SEQ ID NO: 97):
GTGCTTGGTCAGTGTCCAGTTG
Reverse Primer (AA340453_seg24R2 (SEQ ID NO: 98):
GCTCTGAGTTTCCTTCCGGTG
Amplicon (AA340453_seg24F2R2 (SEQ ID NO: 99):
GTGCTTGGTCAGTGTCCAGTTGGCTTTTGGGGGTGTCACAACTACAACAG
AGGCTGAGGCAGTGGGAAAGAGCCCCAAAGAGCTGGAGCTGGCCCACCGG
AAGGAAACTCAGAGC

Expression of Homo sapiens Cadherin 16, KSP-Cadherin (CDH16) AA340453 Transcripts which are Detectable by Amplicon as Depicted in Sequence Name AA340453_seg44 (SEQ ID NO:102) Specifically in Kidney Tissue

Expression of Homo sapiens cadherin 16, KSP-cadherin (CDH16) transcripts detectable by or according to seg44—AA340453_seg44 (SEQ ID NO:102) amplicon and primers AA340453_seg44F (SEQ ID NO:100) and AA340453_seg44R (SEQ ID NO:101) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

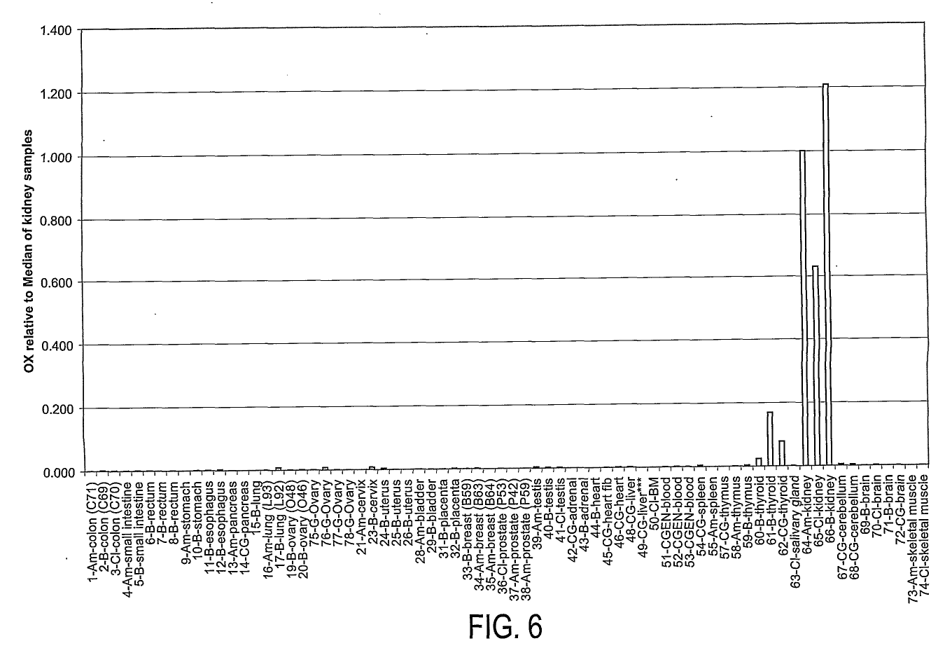

FIG. 6 is a histogram showing relative expression of the above-indicated Homo sapiens cadherin 16, KSP-cadherin (CDH16) transcripts in kidney tissue samples as opposed to other tissues.

As is evident from FIG. 6, the expression of Homo sapiens cadherin 16, KSP-cadherin (CDH16) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: AA340453_seg44F (SEQ ID NO:100) forward primer; and AA340453_seg44R (SEQ ID NO:101) reverse primer.

The present invention also preferably encompasses any amplicon obtained through the use of any suitable primer pair; for example, for the above experiment, the following amplicon was obtained as a non-limiting illustrative example only of a suitable amplicon: AA340453_seg44 (SEQ ID NO:102).

Forward Primer (AA340453_seg44F (SEQ ID NO: 100):
GGACTGAGCTTCCTCCCAGAG
Reverse Primer (AA340453_seg44R (SEQ ID NO: 101):
CCCACCGGAGGAGTAGATCC
Amplicon (AA340453_seg44 (SEQ ID NO: 102):
GGACTGAGCTTCCTCCCAGAGGCCCTCACTGGCAACCCCCAGCCCCAAGC
AGGAGAGGCAGATATGAAGCTGGGTTCAATGTTCCTCTCTGAGAATGGAT
CTACTCCTCCGGTGGG

Expression of Homo sapiens Cadherin 16, KSP-Cadherin (CDH16) AA340453 Transcripts which are Detectable by Amplicon as Depicted in Sequence Name AA340453_seg55WT (SEQ ID NO:83) Specifically in Kidney Tissue

Expression of Homo sapiens cadherin 16, KSP-cadherin (CDH16) transcripts detectable by or according to seg55WT—AA340453_seg55WT (SEQ ID NO:83) amplicon and primers AA340453_seg55WTF (SEQ ID NO:103) and AA340453_seg55WTR (SEQ ID NO:104) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

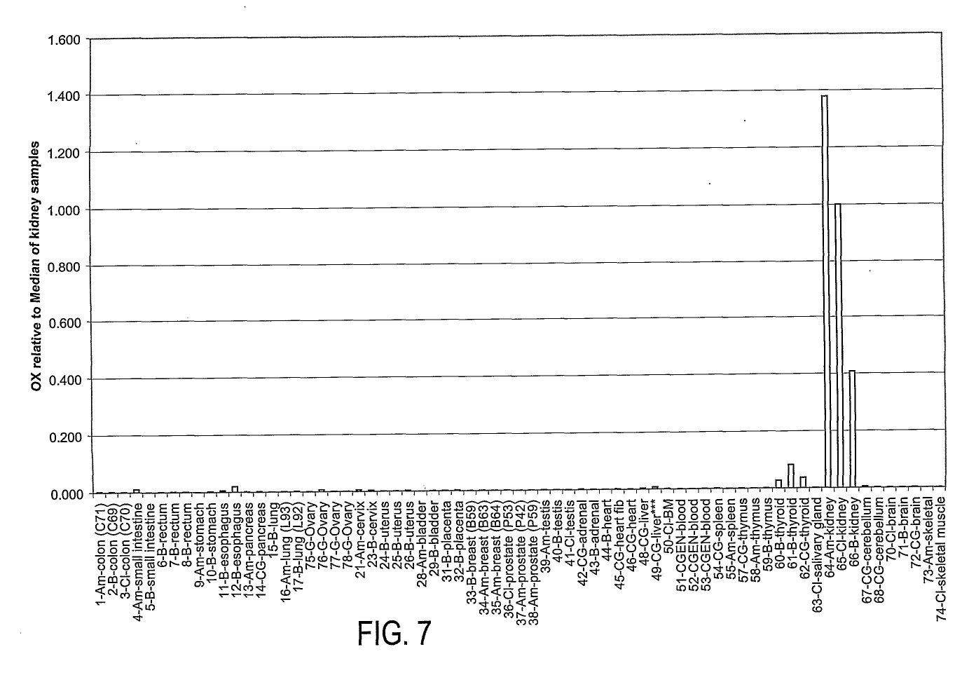

FIG. 7 is a histogram showing relative expression of the above-indicated Homo sapiens cadherin 16, KSP-cadherin (CDH16) transcripts in kidney tissue samples as opposed to other tissues.

As is evident from FIG. 7, the expression of Homo sapiens cadherin 16, KSP-cadherin (CDH16) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: AA340453_seg55WTF (SEQ ID NO:103) forward primer; and AA340453_seg55WTR (SEQ ID NO:104) reverse primer.

The present invention also preferably encompasses any amplicon obtained through the use of any suitable primer pair; for example, for the above experiment, the following amplicon was obtained as a non-limiting illustrative example only of a suitable amplicon: AA340453_seg55WT (SEQ ID NO:83).

Forward Primer (AA340453_seg55WTF (SEQ ID NO:
103):
CATGCCTACCTCACCTTGGC
Reverse Primer (AA340453_seg55WTR (SEQ ID NO:
104):
CTCGAACCAGGAGCTGCC
Amplicon (AA340453_seg55WT (SEQ ID NO: 83):
CATGCCTACCTCACCTTGGCCCTGCATTGGGTGGAGCCACGTGAACACAT
AATCCCCGTGGTGGTCAGCCACAATGCCCAGATGTGGCAGCTCCTGGTTC
GAG

### Description for Cluster AA703666

Cluster AA703666 features 15 transcript(s) and 23 segment(s) of interest, the names for which are given in Tables 34 and 35, respectively. The selected protein variants are given in table 36.

TABLE 34
Transcripts of interest
Transcript Name

AA703666_T1 (SEQ ID NO: 105)
AA703666_T5 (SEQ ID NO: 106)
AA703666_T6 (SEQ ID NO: 107)
AA703666_T7 (SEQ ID NO: 108)
AA703666_T8 (SEQ ID NO: 109)
AA703666_T9 (SEQ ID NO: 110)
AA703666_T11 (SEQ ID NO: 111)
AA703666_T12 (SEQ ID NO: 112)
AA703666_T13 (SEQ ID NO: 113)
AA703666_T14 (SEQ ID NO: 114)
AA703666_T15 (SEQ ID NO: 115)
AA703666_T16 (SEQ ID NO: 116)
AA703666_T17 (SEQ ID NO: 117)
AA703666_T22 (SEQ ID NO: 118)
AA703666_T23 (SEQ ID NO: 119)

TABLE 35
Segments of interest
Segment Name

AA703666_N0 (SEQ ID NO: 120)
AA703666_N9 (SEQ ID NO: 121)
AA703666_N29 (SEQ ID NO: 122)
AA703666_N1 (SEQ ID NO: 123)
AA703666_N4 (SEQ ID NO: 124)
AA703666_N6 (SEQ ID NO: 125)
AA703666_N7 (SEQ ID NO: 126)
AA703666_N11 (SEQ ID NO: 127)
AA703666_N12 (SEQ ID NO: 128)
AA703666_N14 (SEQ ID NO: 129)
AA703666_N16 (SEQ ID NO: 130)
AA703666_N17 (SEQ ID NO: 131)
AA703666_N18 (SEQ ID NO: 132)
AA703666_N19 (SEQ ID NO: 133)
AA703666_N20 (SEQ ID NO: 134)
AA703666_N21 (SEQ ID NO: 135)
AA703666_N22 (SEQ ID NO: 136)
AA703666_N23 (SEQ ID NO: 137)
AA703666_N24 (SEQ ID NO: 138)
AA703666_N25 (SEQ ID NO: 139)
AA703666_N26 (SEQ ID NO: 140)
AA703666_N27 (SEQ ID NO: 141)
AA703666_N28 (SEQ ID NO: 142)

TABLE 36
Proteins of interest

| Protein Name | Corresponding Transcript(s) |
|---|---|
| AA703666_P1 (SEQ ID NO: 144) | AA703666_T1 (SEQ ID NO: 105); |
| | AA703666_T13 (SEQ ID NO: 113) |
| AA703666_P3 (SEQ ID NO: 145) | AA703666_T5 (SEQ ID NO: 106) |
| AA703666_P4 (SEQ ID NO: 146) | AA703666_T6 (SEQ ID NO: 107) |
| AA703666_P5 (SEQ ID NO: 147) | AA703666_T14 (SEQ ID NO: 114); |
| | AA703666_T7 (SEQ ID NO: 108) |
| AA703666_P6 (SEQ ID NO: 148) | AA703666_T8 (SEQ ID NO: 109) |
| AA703666_P7 (SEQ ID NO: 149) | AA703666_T15 (SEQ ID NO: 115); |
| | AA703666_T9 (SEQ ID NO: 110) |
| AA703666_P9 (SEQ ID NO: 150) | AA703666_T11 (SEQ ID NO: 111) |
| AA703666_P10 | AA703666_T17 (SEQ ID NO: 117) |
| (SEQ ID NO: 151) | |
| AA703666_P12 | AA703666_T22 (SEQ ID NO: 118) |
| (SEQ ID NO: 152) | |
| AA703666_P13 | AA703666_T23 (SEQ ID NO: 119) |
| (SEQ ID NO: 153) | |
| AA703666_P15 | AA703666_T12 (SEQ ID NO: 112) |
| (SEQ ID NO: 154) | |
| AA703666_P16 | AA703666_T16 (SEQ ID NO: 116) |
| (SEQ ID NO: 155) | |

These sequences are variants of the known protein Inositol oxygenase (SEQ ID NO:143) (SwissProt accession identifier MIOX_HUMAN (SEQ ID NO:143); known also according to the synonyms EC 1.13.99.1; Myo-inositol oxygenase; Aldehyde reductase-like 6; Renal-specific oxidoreductase; Kidney-specific protein 32), referred to herein as the previously known protein.

Known polymorphisms for this sequence are as shown in Table 37.

TABLE 37
Amino acid mutations for Known Protein

| SNP position(s) on | |
|---|---|
| amino acid sequence | Comment |
| 4 | T -> D |
| 199 | Y -> F |
| 219-221 | FHS -> VHF |
| 282 | I -> T |

Protein Inositol oxygenase (SEQ ID NO:143) localization is believed to be Cytoplasmic (By similarity).

The following GO Annotation(s) apply to the previously known protein. The following annotation(s) were found: cellular osmoregulation; inositol phosphate-mediated signaling; membrane organization and biogenesis; myo-inositol catabolism, which are annotation(s) related to Biological Process; aldo-keto reductase activity; oxidoreductase activity, acting on single donors with incorporation of molecular oxygen, which are annotation(s) related to Molecular Function; and cytoplasm; inclusion body, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

The variant AA703666_P1 (SEQ ID NO:144) was previously disclosed by the inventors in published PCT application no WO2005/071058 and U.S. application Ser. No. 11/043,860; The variants AA703666_P9 (SEQ ID NO:150), AA703666_P10 (SEQ ID NO:151), and AA703666_P12 (SEQ ID NO:152) were previously disclosed by the inventors in published PCT application no WO2005/071058 and WO2004/096979 and U.S. application Ser. No. 11/043,860, 10/242,799 and 10/426,002, hereby incorporated by reference as if fully set forth herein, but have now been shown to have novel and surprising diagnostic uses as described herein for other variants of cluster AA703666.

According to optional but preferred embodiments of the present invention, variants of this cluster according to the present invention (amino acid and/or nucleic acid sequences of AA703666) may optionally have one or more of the following utilities, as described with regard to Table 38 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 38
Table of Utilities for Variants of AA703666, related to
Inositol oxygenase (SEQ ID NO: 143):

| Utility | Reason | Reference |
|---|---|---|
| Renal failure/renal disease | down regulated in acute | 10966922 |
| | ischemic renal failure | |

As noted above, cluster AA703666 features 15 transcript(s), which were listed in Table 34 above. These transcript(s) encode for protein(s) which are variant(s) of protein inositol oxygenase (SEQ ID NO:143). A description of each variant protein according to the present invention is now provided.

Variant protein AA703666_P1 (SEQ ID NO:144) according to the present invention is encoded by transcripts AA703666_T1 (SEQ ID NO:105) and AA703666_T13 (SEQ ID NO:113). One or more alignments to one or more previously published protein sequences are given in the alignment table on the attached CD-ROM. A brief description of the relationship of the variant protein according to the present invention to each such aligned protein is as follows:

1. Comparison report between AA703666_P1 (SEQ ID NO:144) and MIOX_HUMAN (SEQ ID NO:143):

A. An isolated chimeric polypeptide encoding for AA703666_P1 (SEQ ID NO:144), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDK corresponding to amino acids 1-113 of MIOX_HUMAN (SEQ ID NO:143), which also corresponds to amino acids 1-113 of AA703666_P1 (SEQ ID NO:144), and a second amino acid sequence being at least 70%, optionally at least 80%, preferably at least 85%, more preferably at least 90% and most preferably at least 95% homologous to a polypeptide having the sequence VPNLPCPSPSQTGSTSSGSCTTWGRSWPCSGSPSGLSSATPSPSDAVRRPPWFSATPPSRTT LTSRILDTAQSSGCISPTVGSTGSSCPGAMMSTCTR (SEQ ID NO:377) corresponding to amino acids 114-211 of AA703666_P1 (SEQ ID NO:144), wherein said first amino acid sequence and second amino acid sequence are contiguous and in a sequential order.

B. An isolated polypeptide encoding for an edge portion of AA703666_P1 (SEQ ID NO:144), comprising an amino acid sequence being at least 70%, optionally at least about 80%, preferably at least about 85%, more preferably at least about 90% and most preferably at least about 95% homologous to the sequence VPNLPCPSPSQTGSTSSGSCTTWGRSWPCSGSPSGLSSATPSPSDAVRRPPWFSATPPSRTT LTSRILDTAQSSGCISPTVGSTGSSCPGAMMSTCTR (SEQ ID NO:377) of AA703666_P1 (SEQ ID NO:144).

The localization of the variant protein was determined according to results from a number of different software programs and analyses, including analyses from SignalP and other specialized programs. The variant protein is believed to be Cytoplasmic.

Variant protein AA703666_P1 (SEQ ID NO:144) also has the following non-silent SNPs (Single Nucleotide Polymorphisms) as listed in Table 39, (given according to their position(s) on the amino acid sequence, with the alternative amino acid(s) listed).

TABLE 39
Amino acid mutations

| SNP position(s) on amino acid | Alternative |
|---|---|
| sequence | amino acid(s) |
| 146 | P -> L |

Variant protein AA703666_P1 (SEQ ID NO:144) is encoded by transcripts: AA703666_T1 (SEQ ID NO:105) and AA703666_T13 (SEQ ID NO:113).

The coding portion of transcript AA703666_T1 (SEQ ID NO:105) starts at position 199 and ends at position 831. The transcript also has the following SNPs as listed in Table 40 (given according to their position on the nucleotide sequence, with the alternative nucleic acid listed).

TABLE 40
Nucleic acid SNPs

| SNP position(s) on nucleotide | Alternative |
|---|---|
| sequence | nucleic acid(s) |
| 41 | G -> A |
| 468 | C -> T |
| 635 | C -> T |
| 911 | G -> A |
| 932 | G -> A |
| 980 | C -> T |
| 1052 | C -> T |
| 1134 | G -> A |
| 1143 | G -> C |
| 1145 | G -> A |
| 1175 | C -> G |
| 1222 | A -> G |
| 1247 | C -> G |

The coding portion of transcript AA703666_T13 (SEQ ID NO:113) starts at position 199 and ends at position 831. The transcript also has the following SNPs as listed in Table 41 (given according to their position on the nucleotide sequence, with the alternative nucleic acid listed).

TABLE 41
Nucleic acid SNPs

| SNP position(s) on nucleotide | Alternative |
|---|---|
| sequence | nucleic acid(s) |
| 41 | G -> A |
| 468 | C -> T |
| 635 | C -> T |
| 996 | G -> A |
| 1017 | G -> A |
| 1065 | C -> T |
| 1137 | C -> T |
| 1219 | G -> A |
| 1228 | G -> C |
| 1230 | G -> A |
| 1260 | C -> G |
| 1307 | A -> G |
| 1332 | C -> G |

Variant protein AA703666_P3 (SEQ ID NO:145) according to the present invention is encoded by transcript AA703666_T5 (SEQ ID NO:106). One or more alignments to one or more previously published protein sequences are given in the alignment table on the attached CD-ROM. A brief description of the relationship of the variant protein according to the present invention to each such aligned protein is as follows:

1. Comparison report between AA703666_P3 (SEQ ID NO:145) and MIOX_HUMAN (SEQ ID NO:143):

A. An isolated chimeric polypeptide encoding for AA703666_P3 (SEQ ID NO:145), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDKDWF HLVGLLHDLGKVLALFGEPQWAVVGDTFPVGCRPQASVVFCDSTFQDNPDLQDPRYSTE LGMYQPHCGLDRVLMSWGHD corresponding to amino acids 1-195 of MIOX_HUMAN (SEQ ID NO:143), which also corresponds to amino acids 1-195 of AA703666_P3 (SEQ ID NO:145), and a second amino acid sequence being at least 70%, optionally at least 80%, preferably at least 85%, more preferably at least 90% and most preferably at least 95% homologous to a polypeptide having the sequence GEARGGQWGGGGRWGTVGGGGAEAVPAGDTLSPQSTCTR (SEQ ID NO:378) corresponding to amino acids 196-234 of AA703666_P3 (SEQ ID NO:145), wherein said first amino acid sequence and second amino acid sequence are contiguous and in a sequential order.

B. An isolated polypeptide encoding for an edge portion of AA703666_P3 (SEQ ID NO:145), comprising an amino acid sequence being at least 70%, optionally at least about 80%, preferably at least about 85%, more preferably at least about 90% and most preferably at least about 95% homologous to the sequence GEARGGQWGGGGRWGTVGGGGAEAVPAGDTLSPQSTCTR (SEQ ID NO:378) of AA703666_P3 (SEQ ID NO:145).

The localization of the variant protein Was determined according to results from a number of different software programs and analyses, including analyses from SignalP and other specialized programs. The variant protein is believed to be Cytoplasmic.

Variant protein AA703666_P3 (SEQ ID NO:145) is encoded by the transcript AA703666_T5 (SEQ ID NO:106), for which the coding portion starts at position 199 and ends at position 900. The transcript also has the following SNPs as listed in Table 42 (given according to their position on the nucleotide sequence, with the alternative nucleic acid listed).

TABLE 42
Nucleic acid SNPs

| SNP position(s) on nucleotide | |
|---|---|
| sequence | Alternative nucleic acid(s) |
| 41 | G -> A |
| 468 | C -> T |
| 603 | C -> T |
| 980 | G -> A |
| 1001 | G -> A |
| 1049 | C -> T |
| 1121 | C -> T |
| 1203 | G -> A |
| 1212 | G -> C |
| 1214 | G -> A |
| 1244 | C -> G |
| 1291 | A -> G |
| 1316 | C -> G |

Variant protein AA703666_P4 (SEQ ID NO:146) according to the present invention is encoded by transcript AA703666_T6 (SEQ ID NO:107). One or more alignments to one or more previously published protein sequences are given in the alignment table on the attached CD-ROM. A brief description of the relationship of the variant protein according to the present invention to each such aligned protein is as follows:

1. Comparison report between AA703666_P4 (SEQ ID NO:146) and MIOX_HUMAN (SEQ ID NO:143):

A. An isolated chimeric polypeptide encoding for AA703666_P4 (SEQ ID NO:146), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDKDWF HLVGLLHDLGKVLALFGEPQWAVVGDTFPVGCRPQASVVFCDSTFQDNPDLQDPRYSTE LGMYQPHCGLDRVLMSWGHDEYMYQVMKFNKFSLPPE corresponding to amino acids 1-212 of MIOX_HUMAN (SEQ ID NO:143), which also corresponds to amino acids 1-212 of AA703666_P4 (SEQ ID NO:146), and a second amino acid sequence being at least 70%, optionally at least 80%, preferably at least 85%, more preferably at least 90% and most preferably at least 95% homologous to a polypeptide having the sequence VGGGRGNAARPPGPVLQPQVSRWVALPAGFLHDPVPLLLPLAHGPRLPAAVQPAGPGH AALGAGVQQVRPLHQVPGPAGRGQAAALLPGAH (SEQ ID NO:379) corresponding to amino acids 213-303 of AA703666_P4 (SEQ ID NO:146), wherein said first amino acid sequence and second amino acid sequence are contiguous and in a sequential order.

B. An isolated polypeptide encoding for an edge portion of AA703666_P4 (SEQ ID NO:146), comprising an amino acid sequence being at least 70%, optionally at least about 80%, preferably at least about 85%, more preferably at least about 90% and most preferably at least about 95% homologous to the sequence VGGGRGNAARPPGPVLQPQVSRWVALPAGFLHDPVPLLLPLAHGPRLPAAVQPAGPGH AALGAGVQQVRPLHQVPGPAGRGQAAALLPGAH (SEQ ID NO:379) of AA703666_P4 (SEQ ID NO:146).

The localization of the variant protein was determined according to results from a number of different software programs and analyses, including analyses from SignalP and other specialized programs. The variant protein is believed to be Cytoplasmic.

Variant protein AA703666_P4 (SEQ ID NO:146) also has the following non-silent SNPs (Single Nucleotide Polymorphisms) as listed in Table 43, (given according to their position(s) on the amino acid sequence, with the alternative amino acid(s) listed).

TABLE 43
Amino acid mutations

| SNP position(s) on amino acid | |
|---|---|
| sequence | Alternative amino acid(s) |
| 256 | G -> R |
| 263 | V -> M |
| 279 | Q -> * |
| 303 | H -> Y |

Variant protein AA703666_P4 (SEQ ID NO:146) is encoded by the transcript AA703666_T6 (SEQ ID NO:107), for which the coding portion starts at position 199 and ends at position 1107. The transcript also has the following SNPs as listed in Table 44 (given according to their position on the nucleotide sequence, with the alternative nucleic acid listed).

TABLE 44
Nucleic acid SNPs

| SNP position(s) on nucleotide | |
|---|---|
| sequence | Alternative nucleic acid(s) |
| 41 | G -> A |
| 468 | C -> T |
| 603 | C -> T |
| 964 | G -> A |
| 985 | G -> A |
| 1033 | C -> T |
| 1105 | C -> T |
| 1187 | G -> A |
| 1196 | G -> C |
| 1198 | G -> A |
| 1228 | C -> G |
| 1275 | A -> G |
| 1300 | C -> G |

Variant protein AA703666_P5 (SEQ ID NO:147) according to the present invention is encoded by transcripts AA703666_T14 (SEQ ID NO:114) and AA703666_T7 (SEQ ID NO:108). One or more alignments to one or more previously published protein sequences are given in the alignment table on the attached CD-ROM. A brief description of the relationship of the variant protein according to the present invention to each such aligned protein is as follows:

1. Comparison report between AA703666_P5 (SEQ ID NO:147) and MIOX_HUMAN (SEQ ID NO:143):

A. An isolated chimeric polypeptide encoding for AA703666_P5 (SEQ ID NO:147), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDKDWF HLVGLLHDLGKVLALFGEPQWAVVGDTFPVGCRPQASVVFCDSTFQDNPDLQDPRY corresponding to amino acids 1-172 of MIOX_HUMAN (SEQ ID NO:143), which also corresponds to amino acids 1-172 of AA703666_P5 (SEQ ID NO:147), and a second amino acid sequence being at least 70%, optionally at least 80%, preferably at least 85%, more preferably at least 90% and most preferably at least 95% homologous to a polypeptide having the sequence RCSLLLRAGPPSLGGC (SEQ ID NO:380) corresponding to amino acids 173-188 of AA703666_P5 (SEQ ID NO:147), wherein said first amino acid sequence and second amino acid sequence are contiguous and in a sequential order.

B. An isolated polypeptide encoding for an edge portion of AA703666_P5 (SEQ ID NO:147), comprising an amino acid sequence being at least 70%, optionally at least about 80%, preferably at least about 85%, more preferably at least about 90% and most preferably at least about 95% homologous to the sequence RCSLLLRAGPPSLGGC (SEQ ID NO:380) of AA703666_P5 (SEQ ID NO:147).

The localization of the variant protein was determined according to results from a number of different software programs and analyses, including analyses from SignalP and other specialized programs. The variant protein is believed to be Cytoplasmic.

Variant protein AA703666_P5 (SEQ ID NO:147) is encoded by the transcripts AA703666_T14 (SEQ ID NO:114) and AA703666_T7 (SEQ ID NO:108).

The coding portion of transcript AA703666-T14 (SEQ ID NO:114) starts at position 199 and ends at position 762. The transcript also has the following SNPs as listed in Table 45 (given according to their position on the nucleotide sequence, with the alternative nucleic acid listed).

TABLE 45
Nucleic acid SNPs

| SNP position(s) on nucleotide | |
|---|---|
| sequence | Alternative nucleic acid(s) |
| 41 | G -> A |
| 468 | C -> T |
| 603 | C -> T |
| 1058 | G -> A |
| 1079 | G -> A |
| 1127 | C -> T |
| 1199 | C -> T |
| 1281 | G -> A |
| 1290 | G -> C |
| 1292 | G -> A |
| 1322 | C -> G |
| 1369 | A -> G |
| 1394 | C -> G |

The coding portion of transcript AA703666_T7 (SEQ ID NO:108) starts at position 199 and ends at position 762. The transcript also has the following SNPs as listed in Table 46 (given according to their position on the nucleotide sequence, with the alternative nucleic acid listed).

TABLE 46
Nucleic acid SNPs

| SNP position(s) on nucleotide | |
|---|---|
| sequence | Alternative nucleic acid(s) |
| 41 | G -> A |
| 468 | C -> T |
| 603 | C -> T |
| 957 | G -> A |
| 978 | G -> A |
| 1026 | C -> T |
| 1098 | C -> T |
| 1180 | G -> A |
| 1189 | G -> C |
| 1191 | G -> A |
| 1221 | C -> G |
| 1268 | A -> G |
| 1293 | C -> G |

Variant protein AA703666_P6 (SEQ ID NO:148) according to the present invention is encoded by transcript AA703666_T8 (SEQ ID NO:109) One or more alignments to one or more previously published protein sequences are given in the alignment table on the attached CD-ROM. A brief description of the relationship of the variant protein according to the present invention to each such aligned protein is as follows:

1. Comparison report between AA703666_P6 (SEQ ID NO:148) and MIOX_HUMAN (SEQ ID NO:143):

A. An isolated chimeric polypeptide encoding for AA703666_P6 (SEQ ID NO:148), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDKDWF HLVGLLHDLGKVLALFGEPQWAVVGDTFPVGCRPQASVVFCDSTFQDNPDLQDPRYSTE LGMYQPHCGLDRVLMSWGHDEYMYQVMKFNKFSLPPEAFYMIRFHSFYPWHTGRDYQ QLCSQQDLAMLPWVREF corresponding to amino acids 1-249 of MIOX_HUMAN (SEQ ID NO:143), which also corresponds to amino acids 1-249 of AA703666_P6 (SEQ ID NO:148), and a second amino acid sequence being at least 70%, optionally at least 80%, preferably at least 85%, more preferably at least 90% and most preferably at least 95% homologous to a polypeptide having the sequence KYAPLPAEGCCGSERGGWVGGLGVLHGAHRPSLLQQVRPLHQVPGPAGRGQAAALLPG AH (SEQ ID NO:381) corresponding to amino acids 250-309 of AA703666_P6 (SEQ ID NO:148), wherein said first amino acid sequence and second amino acid sequence are contiguous and in a sequential order.

B. An isolated polypeptide encoding for an edge portion of AA703666_P6 (SEQ ID NO:148), comprising an amino acid sequence being at least 70%, optionally at least about 80%, preferably at least about 85%, more preferably at least about 90% and most preferably at least about 95% homologous to the sequence KYAPLPAEGCCGSERGGWVGGLGVLHGAHRPSLLQQVRPLHQVPGPAGRGQAAALLPG AH (SEQ ID NO:381) of AA703666_P6 (SEQ ID NO:148).

The localization of the variant protein was determined according to results from a number of different software programs and analyses, including analyses from SignalP and other specialized programs. The variant protein is believed to: Cytoplasmic.

Variant protein AA703666_P6 (SEQ ID NO:148) also has the following non-silent SNPs (Single Nucleotide Polymorphisms) as listed in Table 47, (given according to their position(s) on the amino acid sequence, with the alternative amino acid(s) listed).

TABLE 47
Amino acid mutations

| SNP position(s) on amino acid | |
|---|---|
| sequence | Alternative amino acid(s) |
| 285 | Q -> * |
| 309 | H -> Y |

Variant protein AA703666_P6 (SEQ ID NO:148) is encoded by the transcript: AA703666_T8 (SEQ ID NO:109), for which the coding portion starts at position 199 and ends at position 1125. The transcript also has the following SNPs as listed in Table 48 (given according to their position on the nucleotide sequence, with the alternative nucleic acid listed).

TABLE 48
Nucleic acid SNPs

| SNP position(s) on nucleotide | |
|---|---|
| sequence | Alternative nucleic acid(s) |
| 41 | G -> A |
| 468 | C -> T |
| 603 | C -> T |
| 879 | G -> A |
| 900 | G -> A |
| 1051 | C -> T |
| 1123 | C -> T |
| 1205 | G -> A |
| 1214 | G -> C |
| 1216 | G -> A |
| 1246 | C -> G |
| 1293 | A -> G |
| 1318 | C -> G |

Variant protein AA703666_P7 (SEQ ID NO:149) according to the present invention is encoded by transcripts AA703666_T15 (SEQ ID NO:115) and AA703666_T9 (SEQ ID NO:110). One or more alignments to one or more previously published protein sequences are given in the alignment table on the attached CD-ROM. A brief description of the relationship of the variant protein according to the present invention to each such aligned; protein is as follows:

1. Comparison report between AA703666_P7 (SEQ ID NO:149) and MIOX_HUMAN (SEQ ID NO:143):

A. An isolated chimeric polypeptide encoding for AA703666_P7 (SEQ ID NO:149), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDKDWF HLVGLLHDLGKVLALFGEPQWAVVGDTFPVGCRPQASVVFCDSTFQDNPDLQDPRYSTE LGMYQPHCGLDRVLMSWGHDEYMYQVMKFNKFSLPPEAFYMIRFHSFYPWHTGRDYQ QLCSQQDLAMLPW corresponding to amino acids 1-245 of MIOX_HUMAN (SEQ ID NO:143), which also corresponds to amino acids 1-245 of AA703666_P7 (SEQ ID NO:149), and a second amino acid sequence being at least 70%, optionally at least 80%, preferably at least 85%, more preferably at least 90% and most preferably at least 95% homologous to a polypeptide having the sequence QVRPLHQVPGPAGRGQAAALLPGAH (SEQ ID NO:382) corresponding to amino acids 246-270 of AA703666_P7 (SEQ ID NO:149), wherein said first amino acid sequence and second amino acid sequence are contiguous and in a sequential order.

B. An isolated polypeptide encoding for an edge portion of AA703666_P7 (SEQ ID NO:149), comprising an amino acid sequence being at least 70%, optionally at least about 80%, preferably at least about 85%, more preferably at least about 90% and most preferably at least about 95% homologous to the sequence QVRPLHQVPGPAGRGQAAALLPGAH (SEQ ID NO:382) of AA703666_P7 (SEQ ID NO:149).

The localization of the variant protein was determined according to results from a number of different software programs and analyses, including analyses from SignalP and other specialized programs. The variant protein is believed to be Cytoplasmic.

Variant protein AA703666_P7 (SEQ ID NO:149) also has the following non-silent SNPs (Single Nucleotide Polymorphisms) as listed in Table 49, (given according to their position(s) on the amino acid sequence, with the alternative amino acid(s) listed).

TABLE 49
Amino acid mutations

| SNP position(s) on amino acid | |
|---|---|
| sequence | Alternative amino acid(s) |
| 246 | Q -> * |
| 270 | H -> Y |

Variant protein AA703666_P7 (SEQ ID NO:149) is encoded by the following transcripts: AA703666_T15 (SEQ ID NO:115) and AA703666_T9 (SEQ ID NO:110).

The coding portion of transcript AA703666_T15 (SEQ ID NO:115) starts at position 199 and ends at position 1008. The transcript also has the following SNPs as listed in Table 50 (given according to their position on the nucleotide sequence, with the alternative nucleic acid listed).

TABLE 50
Nucleic acid SNPs

| SNP position(s) on nucleotide | |
|---|---|
| sequence | Alternative nucleic acid(s) |
| 41 | G -> A |
| 468 | C -> T |
| 603 | C -> T |
| 879 | G -> A |
| 900 | G -> A |
| 934 | C -> T |
| 1006 | C -> T |
| 1088 | G -> A |
| 1097 | G -> C |
| 1129 | C -> G |
| 1176 | A -> G |
| 1201 | C -> G |
| 1322 | G -> A |
| 1409 | G -> A |
| 1751 | T -> C |

The coding portion of transcript AA703666_T9 (SEQ ID NO:110) starts at position 199 and ends at position 1008. The transcript also has the following SNPs as listed in Table 51 (given according to their position on the nucleotide sequence, with the alternative nucleic acid listed).

TABLE 51
Nucleic acid SNPs

| SNP position(s) on nucleotide | |
|---|---|
| sequence | Alternative nucleic acid(s) |
| 41 | G -> A |
| 468 | C -> T |
| 603 | C -> T |
| 879 | G -> A |
| 900 | G -> A |
| 934 | C -> T |
| 1006 | C -> T |
| 1088 | G -> A |
| 1097 | G -> C |
| 1099 | G -> A |
| 1129 | C -> G |
| 1176 | A -> G |
| 1201 | C -> G |

Variant protein AA703666_P9 (SEQ ID NO:150) according to the present invention is encoded by transcript AA703666_T11 (SEQ ID NO:111) One or more alignments to one or more previously published protein sequences are given in the alignment table on the attached CD-ROM. A brief description of the relationship of the variant protein according to the present invention to each such aligned protein is as follows:

1. Comparison report between AA703666_P9 (SEQ ID NO:150) and MIOX_HUMAN (SEQ ID NO:143):

A. An isolated chimeric polypeptide encoding for AA703666_P9 (SEQ ID NO:150), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDKDWF HLVGLLHDLGKVLALFGEPQ corresponding to amino acids 1-136 of MIOX_HUMAN (SEQ ID NO:143), which also corresponds to amino acids 1-136 of AA703666_P9 (SEQ ID NO:150), and a second amino acid sequence being at least 90% homologous to ASVVFCDSTFQDNPDLQDPRYSTELGMYQPHCGLDRVLMSWGHDEYMYQVMKNKFS LPPEAFYMIRFHSFYPWHTGRDYQQLCSQQDLAMLPWVREFNKFDLYTKCPDLPDVDK LRPYYQGLIDKYCPGILSW corresponding to amino acids 152-285 of MIOX_HUMAN (SEQ ID NO:143), which also corresponds to amino acids 137-270 of AA703666_P9 (SEQ ID NO:150), wherein said first amino acid sequence and second amino acid sequence are contiguous and in a sequential order.

B. An isolated chimeric polypeptide encoding for an edge portion of AA703666_P9 (SEQ ID NO:150), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise QA, having a structure as follows: a sequence starting from any of amino acid numbers 136-x to 136; and ending at any of amino acid numbers 137+((n−2)−x), in which x varies from 0 to n−2.

The localization of the variant protein was determined according to results from a number of different software programs and analyses, including analyses from SignalP and other specialized programs. The variant protein is believed to be Cytoplasmic.

Variant protein AA703666_P9 (SEQ ID NO:150) is encoded by the transcript AA703666_T11 (SEQ ID NO:111), for which the coding portion starts at position 199 and ends at position 1008. The transcript also has the following SNPs as listed in Table 52 (given according to their position on the nucleotide sequence, with the alternative nucleic acid listed).

TABLE 52
Nucleic acid SNPs

| SNP position(s) on nucleotide | |
|---|---|
| sequence | Alternative nucleic acid(s) |
| 41 | G -> A |
| 468 | C -> T |
| 603 | C -> T |
| 834 | G -> A |
| 855 | G -> A |
| 903 | C -> T |
| 975 | C -> T |
| 1057 | G -> A |
| 1066 | G -> C |
| 1068 | G -> A |
| 1098 | C -> G |
| 1145 | A -> G |
| 1170 | C -> G |

Variant protein AA703666_P10 (SEQ ID NO:151) according to the present invention is encoded by transcript AA703666_T17 (SEQ ID NO:117) One or more alignments to one or more previously published protein sequences are given in the alignment table on the attached CD-ROM. A brief description of the relationship of the variant protein according to the present invention to each such aligned protein is as follows:

1. Comparison report between AA703666_P10 (SEQ ID NO:151) and MIOX_HUMAN (SEQ ID NO:143):

A. An isolated chimeric polypeptide encoding for AA703666_P10 (SEQ ID NO:151), comprising a first amino acid sequence being at least 90% homologous to MKVTVGPDPSLVYRPDVDPEVAKDKASFRNYT corresponding to amino acids 1-32 of MIOX_HUMAN (SEQ ID NO:143), which also corresponds to amino acids 1-32 of AA703666_P10 (SEQ ID NO:151), and a second amino acid sequence being at least 90% homologous to HAQFGGFSYKKMTVMEAVDLLDGLVDESDPDVDFPNSFHAFQTAEGIRKAHPDKDWFH LVGLLHDLGKVLALFGEPQWAVVGDTFPVGCRPQASVVFCDSTFQDNPDLQDPRYSTEL GMYQPHCGLDRVLMSWGHDEYMYQVMKFNKFSLPPEAFYMIRFHSFYPWHTGRDYQQ LCSQQDLAMLPWVREFNKFDLYTKCPDLPDVDKLRPYYQGLIDKYCPGILSW corresponding to amino acids 60-285 of MIOX_HUMAN (SEQ ID NO:143), which also corresponds to amino acids 33-258 of AA703666_P10 (SEQ ID NO:151), wherein said first amino acid sequence and second amino acid sequence are contiguous and in a sequential order.

B. An isolated chimeric polypeptide encoding for an edge portion of AA703666_P10 (SEQ ID NO:151), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise TH, having a structure as follows: a sequence starting from any of amino acid numbers 32-x to 32; and ending at any of amino acid numbers 33+((n−2)−x), in which x varies from 0 to n−2.

The localization of the variant protein was determined according to results from a number of different software programs and analyses, including analyses from SignalP and other specialized programs. The variant protein is believed to Cytoplasmic.

Variant protein AA703666_P10 (SEQ ID NO:151) is encoded by the transcript AA703666_T17 (SEQ ID NO:117), for which the coding portion starts at position 199 and ends at position 972. The transcript also has the following SNPs as listed in Table 53 (given according to their position on the nucleotide sequence, with the alternative nucleic acid listed).

TABLE 53
Nucleic acid SNPs

| SNP position(s) on nucleotide | |
|---|---|
| sequence | Alternative nucleic acid(s) |
| 41 | G -> A |
| 387 | C -> T |
| 522 | C -> T |
| 798 | G -> A |
| 819 | G -> A |
| 867 | C -> T |
| 939 | C -> T |
| 1021 | G -> A |
| 1030 | G -> C |
| 1032 | G -> A |
| 1062 | C -> G |
| 1109 | A -> G |
| 1134 | C -> G |

Variant protein AA703666_P12 (SEQ ID NO:152) according to the present invention is encoded by transcript AA703666_T22 (SEQ ID NO:118) One or more alignments to one or more previously published protein sequences are given in the alignment table on the attached CD-ROM. A brief description of the relationship of the variant protein according to the present invention to each such aligned protein is as follows:

1. Comparison report between AA703666_P12 (SEQ ID NO:152) and MIOX_HUMAN (SEQ ID NO:143):

A. An isolated chimeric polypeptide encoding for AA703666_P12 (SEQ ID NO:152), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDK corresponding to amino acids 1-113 of MIOX_HUMAN (SEQ ID NO:143), which also corresponds to amino acids 1-113 of AA703666_P12 (SEQ ID NO:152), and a second amino acid sequence being at least 70%, optionally at least 80%, preferably at least 85%, more preferably at least 90% and most preferably at least 95% homologous to a polypeptide having the sequence VPNLPCPSPSQTGSTSSGSCTTWGRSWPCSGSPSGLSSATPSPSDAVRRPPWFSATPPSRTT LTSRILDTAQSSGCISPTVGSTGSSCPGAMMQVRPLHQVPGPAGRGQAAALLPGAH (SEQ ID NO:383) corresponding to amino acids 114-231 of AA703666_P12 (SEQ ID NO:152), wherein said first amino acid sequence and second amino acid sequence are contiguous and in a sequential order.

B. An isolated polypeptide encoding for an edge portion of AA703666_P12 (SEQ ID NO:152), comprising an amino acid sequence being at least 70%, optionally at least about 80%, preferably at least about 85%, more preferably at least about 90% and most preferably at least about 95% homologous to the sequence VPNLPCPSPSQTGSTSSGSCTTWGRSWPCSGSPSGLSSATPSPSDAVRRPPWFSATPPSRTT LTSRILDTAQSSGCISPTVGSTGSSCPGAMMQVRPLHQVPGPAGRGQAAALLPGAH (SEQ ID NO:383) of AA703666_P12 (SEQ ID NO:152).

The localization of the variant protein was determined according to results from a number of different software programs and analyses, including analyses from SignalP and other specialized programs. The variant protein is believed to be Cytoplasmic.

Variant protein AA703666_P12 (SEQ ID NO:152) also has the following non-silent SNPs (Single Nucleotide Polymorphisms) as listed in Table 54, (given according to their position(s) on the amino acid sequence, with the alternative amino acid(s) listed).

TABLE 54
Amino acid mutations

| SNP position(s) on amino acid | |
|---|---|
| sequence | Alternative amino acid(s) |
| 146 | P -> L |
| 207 | Q -> * |
| 231 | H -> Y |

Variant protein AA703666_P12 (SEQ ID NO:152) is encoded by the following transcript AA703666_T22 (SEQ ID NO:118), for which the coding portion starts at position 199 and ends at position 891. The transcript also has the following SNPs as listed in Table 55 (given according to their position on the nucleotide sequence, with the alternative nucleic acid listed).

TABLE 55
Nucleic acid SNPs

| SNP position(s) on nucleotide | |
|---|---|
| sequence | Alternative nucleic acid(s) |
| 41 | G -> A |
| 468 | C -> T |
| 635 | C -> T |
| 817 | C -> T |
| 889 | C -> T |
| 971 | G -> A |
| 980 | G -> C |
| 982 | G -> A |
| 1012 | C -> G |
| 1059 | A -> G |
| 1084 | C -> G |

Variant protein AA703666_P13 (SEQ ID NO:153) according to the present invention is encoded by transcript AA703666_T23 (SEQ ID NO:119). One or more alignments to one or more previously published protein sequences are given in the alignment table on the attached CD-ROM. A brief description of the relationship of the variant protein according to the present invention to each such aligned protein is as follows:

1. Comparison report between AA703666_P13 (SEQ ID NO:153) and MIOX_HUMAN (SEQ ID NO:143):

A. An isolated chimeric polypeptide encoding for AA703666_P13 (SEQ ID NO:153), comprising a first amino acid sequence being at least 90% homologous to MKVTVGPDPSLVYRPDVDPEVAKDKASFRNYTSGPLLDRVFTTYKLMHTHQTVDFVRS KHAQFGGFSYKKMTVMEAVDLLDGLVDESDPDVDFPNSFHAFQTAEGIRKAHPDKDWF HLVGLLHDLGKVLALFGEPQWAV corresponding to amino acids 1-139 of MIOX_HUMAN (SEQ ID NO:143), which also corresponds to amino acids 1-139 of AA703666_P13 (SEQ ID NO:153), and a second amino acid sequence being at least 70%, optionally at least 80%, preferably at least 85%, more preferably at least 90% and most preferably at least 95% homologous to a polypeptide having the sequence IGNIFPHSPHGRPSLLSLCSGQSSVTPSPTPPMAA (SEQ ID NO:384) corresponding to amino acids 140-174 of AA703666_P13 (SEQ ID NO:153), wherein said first amino acid sequence and second amino acid sequence are contiguous and in a sequential order.

B. An isolated polypeptide encoding for an edge portion of AA703666_P13 (SEQ ID NO:153), comprising an amino acid sequence being at least 70%, optionally at least about 80%, preferably at least about 85%, more preferably at least about 90% and most preferably at least about 95% homologous to the sequence IGNIFPHSPHGRPSLLSLCSGQSSVTPSPTPPMAA (SEQ ID NO:384) of AA703666_P13 (SEQ ID NO:153).

The localization of the variant protein was determined according to results from a number of different software programs and analyses, including analyses from SignalP and other specialized programs. The variant protein is believed to be Cytoplasmic.

Variant protein AA703666P113 (SEQ ID NO:153) is encoded by the transcript AA703666_T23 (SEQ ID NO:119), for which the coding portion starts at position 199 and ends at position 720. The transcript also has the following SNPs as listed in Table 56 (given according to their position on the nucleotide sequence, with the alternative nucleic acid listed).

TABLE 56
Nucleic acid SNPs

| SNP position(s) on nucleotide | |
|---|---|
| sequence | Alternative nucleic acid(s) |
| 41 | G -> A |
| 468 | C -> T |
| 603 | C -> T |

Variant protein AA703666_P15 (SEQ ID NO:154) according to the present invention is encoded by transcript AA703666_T12 (SEQ ID NO:112). One or more alignments to one or more previously published protein sequences are given in the alignment table on the attached CD-ROM. A brief description of the relationship of the variant protein according to the present invention to each such aligned protein is as follows:

1. Comparison report between AA703666_P15 (SEQ ID NO:154) and MIOX_HUMAN (SEQ ID NO:143):

A. An isolated chimeric polypeptide encoding for AA703666_P15 (SEQ ID NO:154), comprising a first amino acid sequence being at least 90% homologous to MKVTVGPDPSLVYRPDVDPEVAKDKASFRNYT corresponding to amino acids 1-32 of MIOX_HUMAN (SEQ ID NO:143), which also corresponds to amino acids 1-32 of AA703666_P15 (SEQ ID NO:154), and a second amino acid sequence being at least 70%, optionally at least 80%, preferably at least 85%, more preferably at least 90% and most preferably at least 95% homologous to a polypeptide having the sequence VPSWTVSSPPTSSCTRTRQWTSSGASMPSLGASPTRK (SEQ ID NO:385) corresponding to amino acids 33-69 of AA703666_P15 (SEQ ID NO:154), wherein said first amino acid sequence and second amino acid sequence are contiguous and in a sequential order.

B. An isolated polypeptide encoding for an edge portion of AA703666_P15 (SEQ ID NO:154), comprising an amino acid sequence being at least 70%, optionally at least about 80%, preferably at least about 85%, more preferably at least about 90% and most preferably at least about 95% homologous to the sequence VPSWTVSSPPTSSCTRTRQWTSSGASMPSLGASPTRK (SEQ ID NO:385) of AA703666_P15 (SEQ ID NO:154).

The localization of the variant protein was determined according to results from a number of different software programs and analyses, including analyses from SignalP and other specialized programs. The variant protein is believed to be Cytoplasmic.

Variant protein AA703666_P15 (SEQ ID NO:154) is encoded by the following transcript AA703666_T12 (SEQ ID NO:112), for which the coding portion starts at position 199 and ends at position 405. The transcript also has the following SNPs as listed in Table 57 (given according to their position on the nucleotide sequence, with the alternative nucleic acid listed).

TABLE 57
Nucleic acid SNPs

| SNP position(s) on nucleotide | |
|---|---|
| sequence | Alternative nucleic acid(s) |
| 41 | G -> A |
| 464 | C -> T |
| 599 | C -> T |
| 875 | G -> A |
| 896 | G -> A |
| 944 | C -> T |
| 1016 | C -> T |
| 1098 | G -> A |
| 1107 | G -> C |
| 1109 | G -> A |
| 1139 | C -> G |
| 1186 | A -> G |
| 1211 | C -> G |

Variant protein AA703666_P16 (SEQ ID NO:155) according to the present invention is encoded by transcript AA703666_T16 (SEQ ID NO:116). One or more alignments to one or more previously published protein sequences are given in the alignment table on the attached CD-ROM. A brief description of the relationship of the variant protein according to the present invention to each such aligned protein is as follows:

1. Comparison report between AA703666_P16 (SEQ ID NO:155) and MIOX_HUMAN (SEQ ID NO:143):

A. An isolated chimeric polypeptide encoding for AA703666-P16 (SEQ ID NO:155), comprising a first amino acid sequence of MKVT corresponding to amino acids 1-4 of MIOX_HUMAN (SEQ ID NO:143), which also corresponds to amino acids 1-4 of AA703666_P16 (SEQ ID NO:155), a second amino acid sequence being at least 90% homologous to GPDPSLVYRPDVDPEVAKDKASFRNYT corresponding to amino acids 6-32 of MIOX_HUMAN (SEQ ID NO:143), which also corresponds to amino acids 5-31 of AA703666_P16 (SEQ ID NO:155), and a third amino acid sequence being at least 70%, optionally at least 80%, preferably at least 85%, more preferably at least 90% and most preferably at least 95% homologous to a polypeptide having the sequence VPSWTVSSPPTSSCTRTRQWTSSGASMPSLGASPTRK (SEQ ID NO:385) corresponding to amino acids 32-68 of AA703666_P16 (SEQ ID NO:155), wherein said first amino acid sequence, second amino acid sequence and third amino acid sequence are contiguous and in a sequential order.

C. An isolated polypeptide encoding for an edge portion of AA703666_P16 (SEQ ID NO:155), comprising an amino acid sequence being at least 70%, optionally at least about 80%, preferably at least about 85%, more preferably at least about 90% and most preferably at least about 95% homologous to the sequence VPSWTVSSPPTSSCTRTRQWTSSGASMPSLGASPTRK (SEQ ID NO:385) of AA703666_P16 (SEQ ID NO:155).

The localization of the variant protein was determined according to results from a number of different software programs and analyses, including analyses from SignalP and other specialized programs. The variant protein is believed to be Cytoplasmic.

Variant protein AA703666_P16 (SEQ ID NO:155) is encoded by the transcript AA703666_T16 (SEQ ID NO:116), for which the coding portion starts at position 199 and ends at position 402. The transcript also has the following SNPs as listed in Table 58 (given according to their position on the nucleotide sequence, with the alternative nucleic acid listed).

TABLE 58
Nucleic acid SNPs

| SNP position(s) on nucleotide | |
|---|---|
| sequence | Alternative nucleic acid(s) |
| 41 | G -> A |
| 461 | C -> T |
| 596 | C -> T |
| 872 | G -> A |
| 893 | G -> A |
| 941 | C -> T |
| 1013 | C -> T |
| 1095 | G -> A |
| 1104 | G -> C |
| 1106 | G -> A |
| 1136 | C -> G |
| 1183 | A -> G |
| 1208 | C -> G |

As noted above, cluster AA703666 features 23 segments, which were listed in Table 35 These segments are portions of nucleic acid sequences which are described herein separately because they are of particular interest. A description of some of these segments according to the present invention is now provided.

Segment cluster AA703666_N0 (SEQ ID NO:120) according to the present invention is supported by 22 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T17 (SEQ ID NO:117), AA703666_T22 (SEQ ID NO:118), AA703666_T23 (SEQ ID NO:119), AA703666_T5 (SEQ ID NO:106), AA703666_T6 (SEQ ID NO:107), AA703666_T7 (SEQ ID NO:108), AA703666_T8 (SEQ ID No:109) and AA703666_T9 (SEQ ID NO:110). Table 59 below describes the starting and ending position of this segment on each transcript.

TABLE 59
Segment location on transcripts

| | Segment | Segment |
|---|---|---|
| Transcript name | starting position | ending position |
| AA703666_T1 (SEQ ID NO: 105) | 1 | 210 |
| AA703666_T11 (SEQ ID NO: 111) | 1 | 210 |
| AA703666_T12 (SEQ ID NO: 112) | 1 | 210 |
| AA703666_T13 (SEQ ID NO: 113) | 1 | 210 |
| AA703666_T14 (SEQ ID NO: 114) | 1 | 210 |
| AA703666_T15 (SEQ ID NO: 115) | 1 | 210 |
| AA703666_T16 (SEQ ID NO: 116) | 1 | 210 |
| AA703666_T17 (SEQ ID NO: 117) | 1 | 210 |
| AA703666_T22 (SEQ ID NO: 118) | 1 | 210 |
| AA703666_T23 (SEQ ID NO: 119) | 1 | 210 |
| AA703666_T5 (SEQ ID NO: 106) | 1 | 210 |
| AA703666_T6 (SEQ ID NO: 107) | 1 | 210 |
| AA703666_T7 (SEQ ID NO: 108) | 1 | 210 |
| AA703666_T8 (SEQ ID NO: 109) | 1 | 210 |
| AA703666_T9 (SEQ ID NO: 110) | 1 | 210 |

Segment cluster AA703666_N9 (SEQ ID NO:121) according to the present invention is supported by 32 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T17 (SEQ ID NO:117), AA703666_T22 (SEQ ID NO:118), AA703666_T23 (SEQ ID NO:119), AA703666_T5 (SEQ ID NO:106), AA703666_T6 (SEQ ID NO:107), AA703666_T7 (SEQ ID NO:108), AA703666_T8 (SEQ ID NO:109) and AA703666_T9 (SEQ ID NO:110). Table 60 below describes the starting and ending position of this segment on each transcript.

TABLE 60
Segment location on transcripts

| | Segment | Segment |
|---|---|---|
| Transcript name | starting position | ending position |
| AA703666_T1 (SEQ ID NO: 105) | 376 | 538 |
| AA703666_T11 (SEQ ID NO: 111) | 376 | 538 |
| AA703666_T12 (SEQ ID NO: 112) | 372 | 534 |
| AA703666_T13 (SEQ ID NO: 113) | 376 | 538 |
| AA703666_T14 (SEQ ID NO: 114) | 376 | 538 |
| AA703666_T15 (SEQ ID NO: 115) | 376 | 538 |
| AA703666_T16 (SEQ ID NO: 116) | 369 | 531 |
| AA703666_T17 (SEQ ID NO: 117) | 295 | 457 |
| AA703666_T22 (SEQ ID NO: 118) | 376 | 538 |
| AA703666_T23 (SEQ ID NO: 119) | 376 | 538 |
| AA703666_T5 (SEQ ID NO: 106) | 376 | 538 |
| AA703666_T6 (SEQ ID NO: 107) | 376 | 538 |
| AA703666_T7 (SEQ ID NO: 108) | 376 | 538 |
| AA703666_T8 (SEQ ID NO: 109) | 376 | 538 |
| AA703666_T9 (SEQ ID NO: 110) | 376 | 538 |

According to an optional embodiment of the present invention, short segments related to the above cluster are also provided. These segments are up to about 120 bp in length, and so are included in a separate description.

Segment cluster AA703666_N4 (SEQ ID NO:124) according to the present invention is supported by 27 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T17 (SEQ ID NO:117), AA703666_T22 (SEQ ID NO:118), AA703666_T23 (SEQ ID NO:119), AA703666_T5 (SEQ ID NO:106), AA703666_T6 (SEQ ID NO:107), AA703666_T7 (SEQ ID NO:108), AA703666_T8 (SEQ ID NO:109) and AA703666_T9 (SEQ ID NO:110). Table 61 below describes the starting and ending position of this segment on each transcript.

TABLE 61
Segment location on transcripts

| | Segment | Segment |
|---|---|---|
| Transcript name | starting position | ending position |
| AA703666_T1 (SEQ ID NO: 105) | 214 | 294 |
| AA703666_T11 (SEQ ID NO: 111) | 214 | 294 |
| AA703666_T12 (SEQ ID NO: 112) | 214 | 294 |
| AA703666_T13 (SEQ ID NO: 113) | 214 | 294 |
| AA703666_T14 (SEQ ID NO: 114) | 214 | 294 |
| AA703666_T15 (SEQ ID NO: 115) | 214 | 294 |
| AA703666_T16 (SEQ ID NO: 116) | 211 | 291 |
| AA703666_T17 (SEQ ID NO: 117) | 214 | 294 |
| AA703666_T22 (SEQ ID NO: 118) | 214 | 294 |
| AA703666_T23 (SEQ ID NO: 119) | 214 | 294 |
| AA703666_T5 (SEQ ID NO: 106) | 214 | 294 |
| AA703666_T6 (SEQ ID NO: 107) | 214 | 294 |
| AA703666_T7 (SEQ ID NO: 108) | 214 | 294 |
| AA703666_T8 (SEQ ID NO: 109) | 214 | 294 |
| AA703666_T9 (SEQ ID NO: 110) | 214 | 294 |

Segment cluster AA703666_N7 (SEQ ID NO: 126) according to the present invention is supported by 26 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T22 (SEQ ID NO:118), AA703666_T23 (SEQ ID NO:119), AA703666_T5 (SEQ ID NO:106), AA703666_T6 (SEQ ID NO:107), AA703666_T7 (SEQ ID NO:108), AA703666_T8 (SEQ ID NO:109) and AA703666_T9 (SEQ ID NO:110). Table 62 below describes the starting and ending position of this segment on each transcript.

TABLE 62
Segment location on transcripts

| | Segment | Segment |
|---|---|---|
| Transcript name | starting position | ending position |
| AA703666_T1 (SEQ ID NO: 105) | 299 | 375 |
| AA703666_T11 (SEQ ID NO: 111) | 299 | 375 |
| AA703666_T12 (SEQ ID NO: 112) | 295 | 371 |
| AA703666_T13 (SEQ ID NO: 113) | 299 | 375 |
| AA703666_T14 (SEQ ID NO: 114) | 299 | 375 |
| AA703666_T15 (SEQ ID NO: 115) | 299 | 375 |
| AA703666_T16 (SEQ ID NO: 116) | 292 | 368 |
| AA703666_T22 (SEQ ID NO: 118) | 299 | 375 |
| AA703666_T23 (SEQ ID NO: 119) | 299 | 375 |
| AA703666_T5 (SEQ ID NO: 106) | 299 | 375 |
| AA703666_T6 (SEQ ID NO: 107) | 299 | 375 |
| AA703666_T7 (SEQ ID NO: 108) | 299 | 375 |
| AA703666_T8 (SEQ ID NO: 109) | 299 | 375 |
| AA703666_T9 (SEQ ID NO: 110) | 299 | 375 |

Segment cluster AA703666_N11 (SEQ ID NO:127) according to the present invention is supported by 5 libraries. The number of libraries was determined as previously described. This segment can be found in the following transcript(s): AA703666_T1 (SEQ ID NO:105), AA703666_T13 (SEQ ID NO:113) and AA703666_T22 (SEQ ID NO:118). Table 63 below describes the starting and ending position of this segment on each transcript.

TABLE 63
Segment location on transcripts

| | Segment | Segment |
|---|---|---|
| Transcript name | starting position | ending position |
| AA703666_T1 (SEQ ID NO: 105) | 539 | 570 |
| AA703666_T13 (SEQ ID NO: 113) | 539 | 570 |
| AA703666_T22 (SEQ ID NO: 118) | 539 | 570 |

Segment cluster AA703666_N12 (SEQ ID NO:128) according to the present invention is supported by 30 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T17 (SEQ ID NO:117), AA703666_T22 (SEQ ID NO:118), AA703666_T23 (SEQ ID NO:119), AA703666_T5 (SEQ ID NO:106), AA703666_T6 (SEQ ID NO:107), AA703666_T7 (SEQ ID NO:108), AA703666_T8 (SEQ ID NO:109) and AA703666_T9 (SEQ ID NO:110). Table 64 below describes the starting and ending position of this segment on each transcript.

TABLE 64
Segment location on transcripts

| | Segment | Segment |
|---|---|---|
| Transcript name | starting position | ending position |
| AA703666_T1 (SEQ ID NO: 105) | 571 | 638 |
| AA703666_T11 (SEQ ID NO: 111) | 539 | 606 |
| AA703666_T12 (SEQ ID NO: 112) | 535 | 602 |
| AA703666_T13 (SEQ ID NO: 113) | 571 | 638 |
| AA703666_T14 (SEQ ID NO: 114) | 539 | 606 |
| AA703666_T15 (SEQ ID NO: 115) | 539 | 606 |
| AA703666_T16 (SEQ ID NO: 116) | 532 | 599 |
| AA703666_T17 (SEQ ID NO: 117) | 458 | 525 |
| AA703666_T22 (SEQ ID NO: 118) | 571 | 638 |
| AA703666_T23 (SEQ ID NO: 119) | 539 | 606 |
| AA703666_T5 (SEQ ID NO: 106) | 539 | 606 |
| AA703666_T6 (SEQ ID NO: 107) | 539 | 606 |
| AA703666_T7 (SEQ ID NO: 108) | 539 | 606 |
| AA703666_T8 (SEQ ID NO: 109) | 539 | 606 |
| AA703666_T9 (SEQ ID NO: 110) | 539 | 606 |

Segment cluster AA703666_N14 (SEQ ID NO:129) according to the present invention is supported by 1 libraries. The number of libraries was determined as previously described. This segment can be found in the following transcript(s): AA703666_T23 (SEQ ID NO:119). Table 65 below describes the starting and ending position of this segment on each transcript.

TABLE 65
Segment location on transcripts

| | Segment | Segment |
|---|---|---|
| Transcript name | starting position | ending position |
| AA703666_T23 (SEQ ID NO: 119) | 607 | 721 |

Segment cluster AA703666_N17 (SEQ ID NO:131) according to the present invention is supported by 27 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T17 (SEQ ID NO:117), AA703666_T22 (SEQ ID NO:118), AA703666_T5 (SEQ ID NO:106), AA703666_T6 (SEQ ID NO:107), AA703666_T7 (SEQ ID NO:108), AA703666_T8 (SEQ ID NO:109) and AA703666_T9 (SEQ ID NO:110). Table 66 below describes the starting and ending position of this segment on each transcript.

TABLE 66
Segment location on transcripts

| | Segment | Segment |
|---|---|---|
| Transcript name | starting position | ending position |
| AA703666_T1 (SEQ ID NO: 105) | 684 | 748 |
| AA703666_T11 (SEQ ID NO: 111) | 607 | 671 |
| AA703666_T12 (SEQ ID NO: 112) | 648 | 712 |
| AA703666_T13 (SEQ ID NO: 113) | 684 | 748 |
| AA703666_T14 (SEQ ID NO: 114) | 652 | 716 |
| AA703666_T15 (SEQ ID NO: 115) | 652 | 716 |
| AA703666_T16 (SEQ ID NO: 116) | 645 | 709 |
| AA703666_T17 (SEQ ID NO: 117) | 571 | 635 |
| AA703666_T22 (SEQ ID NO: 118) | 684 | 748 |
| AA703666_T5 (SEQ ID NO: 106) | 652 | 716 |
| AA703666_T6 (SEQ ID NO: 107) | 652 | 716 |
| AA703666_T7 (SEQ ID NO: 108) | 652 | 716 |
| AA703666_T8 (SEQ ID NO: 109) | 652 | 716 |
| AA703666_T9 (SEQ ID NO: 110) | 652 | 716 |

Segment cluster AA703666_N18 (SEQ ID NO:132) according to the present invention is supported by 4 libraries. The number of libraries was determined as previously described. This segment can be found in the following transcript(s): AA703666_T14 (SEQ ID NO:114) and AA703666_T7 (SEQ ID NO:108). Table 67 below describes the starting and ending position of this segment on each transcript.

TABLE 67
Segment location on transcripts

| | Segment | Segment |
|---|---|---|
| Transcript name | starting position | ending position |
| AA703666_T14 (SEQ ID NO: 114) | 717 | 794 |
| AA703666_T7 (SEQ ID NO: 108) | 717 | 794 |

Segment cluster AA703666_N19 (SEQ ID NO:133) according to the present invention is supported by 24 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T17 (SEQ ID NO:117), AA703666_T22 (SEQ ID NO:118), AA703666_T5 (SEQ ID NO:106), AA703666_T6 (SEQ ID NO:107), AA703666_T7 (SEQ ID NO:108), AA703666_T8 (SEQ ID NO:109) and AA703666_T9 (SEQ ID NO:110). Table 68 below describes the starting and ending position of this segment on each transcript.

TABLE 68
Segment location on transcripts

| | Segment | Segment |
|---|---|---|
| Transcript name | starting position | ending position |
| AA703666_T1 (SEQ ID NO: 105) | 749 | 816 |
| AA703666_T11 (SEQ ID NO: 111) | 672 | 739 |
| AA703666_T12 (SEQ ID NO: 112) | 713 | 780 |
| AA703666_T13 (SEQ ID NO: 113) | 749 | 816 |
| AA703666_T14 (SEQ ID NO: 114) | 795 | 862 |
| AA703666_T15 (SEQ ID NO: 115) | 717 | 784 |
| AA703666_T16 (SEQ ID NO: 116) | 710 | 777 |
| AA703666_T17 (SEQ ID NO: 117) | 636 | 703 |
| AA703666_T22 (SEQ ID NO: 118) | 749 | 816 |
| AA703666_T5 (SEQ ID NO: 106) | 717 | 784 |
| AA703666_T6 (SEQ ID NO: 107) | 717 | 784 |
| AA703666_T7 (SEQ ID NO: 108) | 795 | 862 |
| AA703666_T8 (SEQ ID NO: 109) | 717 | 784 |
| AA703666_T9 (SEQ ID NO: 110) | 717 | 784 |

Segment cluster AA703666_N20 (SEQ ID NO:134) according to the present invention is supported by 8 libraries. The number of libraries was determined as previously described. This segment can be found in the following transcript(s): AA703666_T14 (SEQ ID NO:114) and AA703666_T5 (SEQ ID NO:106). Table 69 below describes the starting and ending position of this segment on each transcript.

TABLE 69
Segment location on transcripts

| | Segment | Segment |
|---|---|---|
| Transcript name | starting position | ending position |
| AA703666_T14 (SEQ ID NO: 114) | 863 | 963 |
| AA703666_T5 (SEQ ID NO: 106) | 785 | 885 |

Segment cluster AA703666_N23 (SEQ ID NO:137) according to the present invention is supported by 5 libraries. The number of libraries was determined as previously described. This segment can be found in the following transcript(s): AA703666_T13 (SEQ ID NO:113) and AA703666_T6 (SEQ. ID NO:107). Table 70 below describes the starting and ending position of this segment on each transcript.

TABLE 70
Segment location on transcripts

| | Segment | Segment |
|---|---|---|
| Transcript name | starting position | ending position |
| AA703666_T13 (SEQ ID NO: 113) | 867 | 951 |
| AA703666_T6 (SEQ ID NO: 107) | 835 | 919 |

Segment cluster AA703666_N25 (SEQ ID NO:139) according to the present invention is supported by 28 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T17 (SEQ ID NO:117), AA703666_T5 (SEQ ID NO:106), AA703666_T6 (SEQ ID NO:107), AA703666_T7 (SEQ ID NO:108), AA703666_T8 (SEQ ID NO:109) and AA703666_T9 (SEQ ID NO:110). Table 71 below describes the starting and ending position of this segment on each transcript.

TABLE 71
Segment location on transcripts

| | Segment | Segment |
|---|---|---|
| Transcript name | starting position | ending position |
| AA703666_T1 (SEQ ID NO: 105) | 913 | 965 |
| AA703666_T11 (SEQ ID NO: 111) | 836 | 888 |
| AA703666_T12 (SEQ ID NO: 112) | 877 | 929 |
| AA703666_T13 (SEQ ID NO: 113) | 998 | 1050 |
| AA703666_T14 (SEQ ID NO: 114) | 1060 | 1112 |
| AA703666_T15 (SEQ ID NO: 115) | 881 | 933 |
| AA703666_T16 (SEQ ID NO: 116) | 874 | 926 |
| AA703666_T17 (SEQ ID NO: 117) | 800 | 852 |
| AA703666_T5 (SEQ ID NO: 106) | 982 | 1034 |
| AA703666_T6 (SEQ ID NO: 107) | 966 | 1018 |
| AA703666_T7 (SEQ ID NO: 108) | 959 | 1011 |
| AA703666_T8 (SEQ ID NO: 109) | 881 | 933 |
| AA703666_T9 (SEQ ID NO: 110) | 881 | 933 |

Segment cluster AA703666_N27 (SEQ ID NO:141) according to the present invention is supported by 7 libraries. The number of libraries was determined as previously described. This segment can be found in the following transcript(s): AA703666_T8 (SEQ ID NO:109). Table 72 below describes the starting and ending position of this segment on each transcript.

TABLE 72
Segment location on transcripts

| | Segment | Segment |
|---|---|---|
| Transcript name | starting position | ending position |
| AA703666_T8 (SEQ ID NO: 109) | 948 | 1050 |

Segment cluster AA703666_N28 (SEQ ID NO:142) according to the present invention is supported by 31 libraries. The number of libraries was determined as previously described. This segment can be found in the following transcript(s): AA703666_T1 (SEQ ID NO:105), AA703666_T11 (SEQ ID NO:111), AA703666_T12 (SEQ ID NO:112), AA703666_T13 (SEQ ID NO:113), AA703666_T14 (SEQ ID NO:114), AA703666_T15 (SEQ ID NO:115), AA703666_T16 (SEQ ID NO:116), AA703666_T17 (SEQ ID NO:117), AA703666_T22 (SEQ ID NO:118), AA703666_T5 (SEQ ID NO:106), AA703666_T6 (SEQ ID NO:107), AA703666_T7 (SEQ ID NO:108), AA703666_T8 (SEQ ID NO:109) and AA703666_T9 (SEQ ID NO:110). Table 73 below describes the starting and ending position of this segment on each transcript.

TABLE 73
Segment location on transcripts

| | Segment | Segment |
|---|---|---|
| Transcript name | starting position | ending position |
| AA703666_T1 (SEQ ID NO: 105) | 980 | 982 |
| AA703666_T11 (SEQ ID NO: 111) | 903 | 905 |
| AA703666_T12 (SEQ ID NO: 112) | 944 | 946 |
| AA703666_T13 (SEQ ID NO: 113) | 1065 | 1067 |
| AA703666_T14 (SEQ ID NO: 114) | 1127 | 1129 |
| AA703666_T15 (SEQ ID NO: 115) | 934 | 936 |
| AA703666_T16 (SEQ ID NO: 116) | 941 | 943 |
| AA703666_T17 (SEQ ID NO: 117) | 867 | 869 |
| AA703666_T22 (SEQ ID NO: 118) | 817 | 819 |
| AA703666_T5 (SEQ ID NO: 106) | 1049 | 1051 |
| AA703666_T6 (SEQ ID NO: 107) | 1033 | 1035 |
| AA703666_T7 (SEQ ID NO: 108) | 1026 | 1028 |
| AA703666_T8 (SEQ ID NO: 109) | 1051 | 1053 |
| AA703666_T9 (SEQ ID NO: 110) | 934 | 936 |

Expression of Homo sapiens aldehyde reductase (aldose reductase) like 6 (ALDRL6) AA703666 transcripts which are detectable by amplicon as depicted in sequence name AA703666_junc12-17 (SEQ ID NO:158) specifically in kidney tissue

Expression of Homo sapiens aldehyde reductase (aldose reductase) like 6 (ALDRL6) transcripts detectable by or according to junc12-17—AA703666_junc12-17 (SEQ ID NO:158) amplicon and primers AA703666_junc12-17F (SEQ ID NO:156) and AA703666juncl2-17R (SEQ ID NO:157) was measured by real time PCR. In parallel the expression of four housekeeping genes—SDHA (GenBank Accession No. NM_{—}004168 (SEQ ID NO:33); amplicon —SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

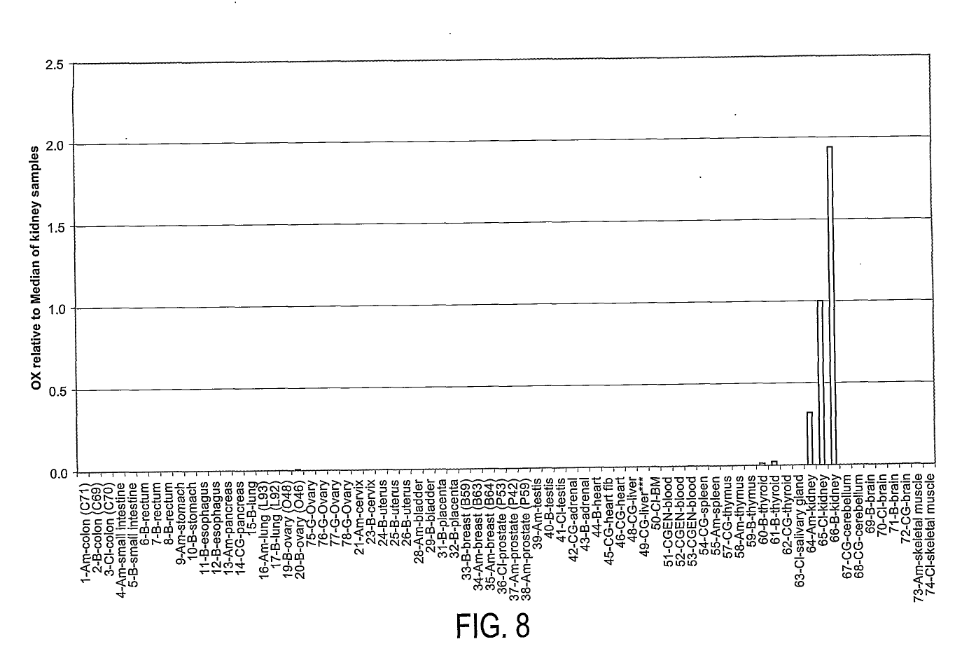

FIG. 8 is a histogram showing relative expression of the above-indicated Homo sapiens aldehyde reductase (aldose reductase) like 6 (ALDRL6) transcripts in kidney tissue samples as opposed to other tissues.

As is evident from FIG. 8, the expression of Homo sapiens aldehyde reductase (aldose reductase) like 6 (ALDRL6) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: AA703666junc12-17F (SEQ ID NO:156) forward primer; and AA703666junc12-17R (SEQ ID NO:157) reverse primer.

The present invention also preferably encompasses any amplicon obtained through the use of any suitable primer pair; for example, for the above experiment, the following amplicon was obtained as a non-limiting illustrative example only of a suitable amplicon: AA703666junc12-17 (SEQ ID NO:158).

Forward Primer (AA703666_junc12-17F
(SEQ ID NO: 156):
GGGAGCCCCAGGCCTC
Reverse Primer (AA703666_junc12-17R
(SEQ ID NO: 157):
CACAGTGGGGCTGATACATCC
Amplicon (AA703666_junc12-17 (SEQ ID NO: 158):
GGGAGCCCCAGGCCTCCGTGGTTTTCTGCGACTCCACCTTCCAGGACAAC
CCTGACCTCCAGGATCCTCGATACAGCACAGAGCTCGGGATGTATCAGCC
CCACTGTG

Expression of Homo sapiens aldehyde reductase (aldose reductase) like 6 (ALDRL6) AA703666 transcripts which are detectable by amplicon as depicted in sequence name AA703666_seg9WT (SEQ ID NO:161) specifically in kidney tissue

Expression of Homo sapiens aldehyde reductase (aldose reductase) like 6 (ALDRL6) transcripts detectable by or according to seg9WT—AA703666_seg9WT (SEQ ID NO:161) amplicon and primers AA703666_seg9WTF (SEQ ID NO:159) and AA703666_seg9WTR (SEQ ID NO:160) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 65 and 66, Table 1_{—}5 above), to obtain a value of relative expression of each sample relative to median of the kidney samples.

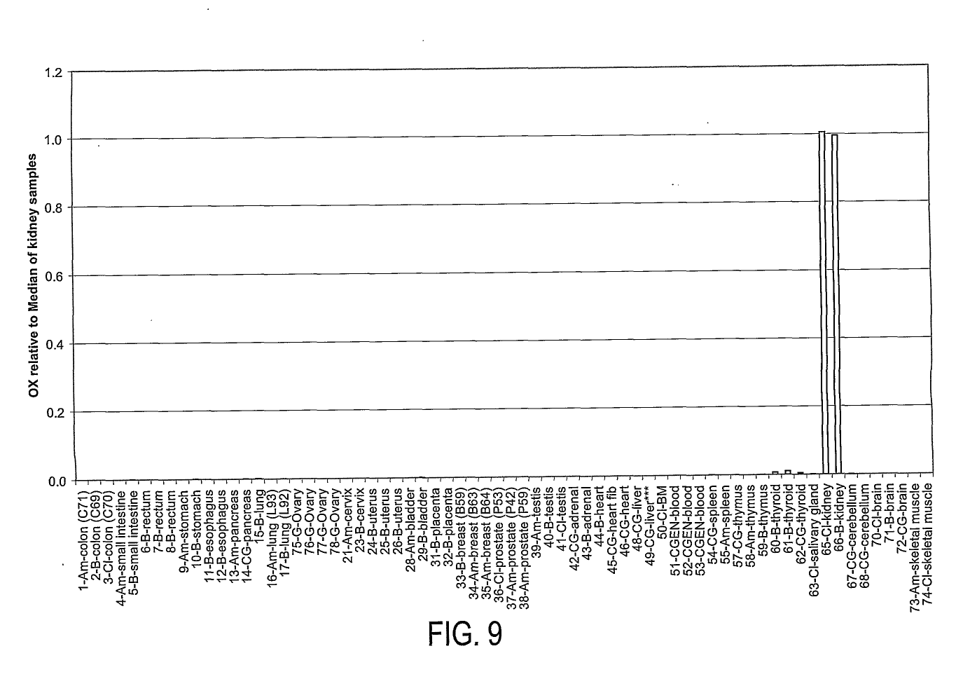

FIG. 9 is a histogram showing relative expression of the above-indicated Homo sapiens aldehyde reductase (aldose reductase) like 6 (ALDRL6) transcripts in kidney tissue samples as opposed to other tissues.

As is evident from FIG. 9, the expression of Homo sapiens aldehyde reductase (aldose reductase) like 6 (ALDRL6) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: AA703666_seg9WTF (SEQ ID NO:159) forward primer; and AA703666_seg9WTR (SEQ ID NO:160) reverse primer.

The present invention also preferably encompasses any amplicon obtained through the use of any suitable primer pair; for example, for the above experiment, the following amplicon was obtained as a non-limiting illustrative example only of a suitable amplicon: AA703666_seg9WT (SEQ ID NO:161).

Forward Primer (AA703666_seg9WTF (SEQ ID NO: 159):
TGACAGTCATGGAGGCCGT
Reverse Primer (AA703666_seg9WTR (SEQ ID NO: 160):
CTCCGCTGTCTGGAAGGC
Amplicon (AA703666_seg9WT (SEQ ID NO: 161):
TGACAGTCATGGAGGCCGTGGACCTGCTGGATGGGCTGGTGGATGAGTCG
GACCCGGACGTAGATTTCCCCAACTCCTTCCATGCCTTCCAGACAGCGGA
G

Expression of Homo sapiens aldehyde reductase (aldose reductase) like 6 (ALDRL6) AA703666 transcripts which are detectable by amplicon as depicted in sequence name AA703666_seg18 (SEQ ID NO:164) specifically in kidney tissue

Expression of Homo sapiens aldehyde reductase (aldose reductase) like 6 (ALDRL6) transcripts detectable by or according to seg18—AA703666_seg18 (SEQ ID NO:164) amplicon and primers AA703666_seg18F (SEQ ID NO:162) and AA703666_seg18R (SEQ ID NO:163) was measured by real time PCR. In parallel the expression of four housekeeping genes—SDHA (GenBank Accession No. NM_{—}004168 (SEQ ID NO:33); amplicon—SDHA-amplicon (SEQ ID NO:36)), Ubiquitin (GenBank Accession No. BC000449 (SEQ ID NO:29); amplicon—Ubiquitin-amplicon (SEQ ID NO:32)), RPL19 (GenBank Accession No. NM_{—}000981 (SEQ ID NO:21); RPL19 amplicon (SEQ ID NO:24)) and TATA box (GenBank Accession No. NM_{—}003194 (SEQ ID NO:25); TATA amplicon (SEQ ID NO:28)) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

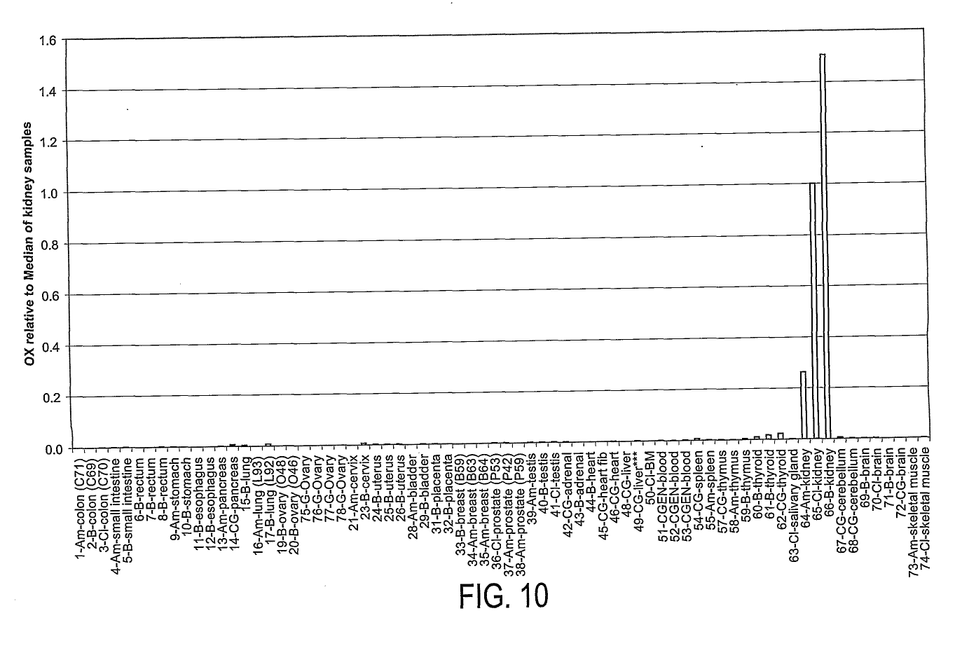

FIG. 10 is a histogram showing relative expression of the above-indicated Homo sapiens aldehyde reductase (aldose reductase) like 6 (ALDRL6) transcripts in kidney tissue samples as opposed to other tissues.

As is evident from FIG. 10, the expression of Homo sapiens aldehyde reductase (aldose reductase) like 6 (ALDRL6) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: AA703666_seg18F (SEQ ID NO:162) forward primer; and AA703666_seg18R (SEQ ID NO:163) reverse primer.

The present invention also preferably encompasses any amplicon obtained through the use of any suitable primer pair; for example, for the above experiment, the following amplicon was obtained as a non-limiting illustrative example only of a suitable amplicon: AA703666_seg18 (SEQ ID NO:164).

Forward Primer (AA703666_seg18F (SEQ ID NO: 162):
TCCTCGATACAGGTGCTCCC
Reverse Primer (AA703666_seg18R (SEQ ID NO: 163):
CCGAGCTCTGTGCTGCAGT
Amplicon (AA703666_seg18 (SEQ ID NO: 164):
TCCTCGATACAGGTGCTCCCTGCTGCTGAGGGCTGGGCCCCCTTCCCTGG
GCGGCTGCTGAGCCCTCCTCACCCTGGTATCTCACTGCAGCACAGAGCTC
GG

Expression of Homo sapiens aldehyde reductase (aldose reductase) like 6 (ALDRL6) AA7030666 transcripts which are detectable by amplicon as depicted in sequence name AA7030666junc4-7F2R2 (SEQ ID NO:440) specifically in kidney tissue

Expression of Homo sapiens aldehyde reductase (aldose reductase) like 6 (ALDRL6) transcripts detectable by or according to junc4-7F2R2—AA7030666junc4-7F2R2 amplicon (SEQ ID NO:440) and primers AA7030666_junc4-7F2 (SEQ ID NO:438) and AA7030666_junc4-7R2 (SEQ ID NO:439) was measured by real time PCR. Non-detected samples (samples no. 3, 11, 16, 17, 25, 26, 28, 32, 33, 39, 40, 41, 42 and 49, Table 1_{—}6) were assigned Ct value of 41 and were calculated accordingly. In parallel the expression of several housekeeping genes—SDHA (GenBank Accession No. NM_{—}004168 (SEQ ID NO:33); amplicon—SDHA-amplicon (SEQ ID NO:36)), Ubiquitin (GenBank Accession No. BC000449 (SEQ ID NO:29); amplicon—Ubiquitin-amplicon (SEQ ID NO:32)) and TATA box (GenBank Accession No. NM_{—}003194 (SEQ ID NO:25); TATA amplicon (SEQ ID NO:28)) was measured similarly. For each RT sample, the expression of the above amplicon was normalized to the normalization factor calculated from the expression of these house keeping genes as described in normalization method 2 in the “materials and methods” section. The normalized quantity of each RT sample was then divided by the median of the quantities of the kidney samples (sample numbers 19, 20, 21, 22 and 23, Table 1_{—}6 above), to obtain a value of relative expression of each sample relative to median of the kidney samples.

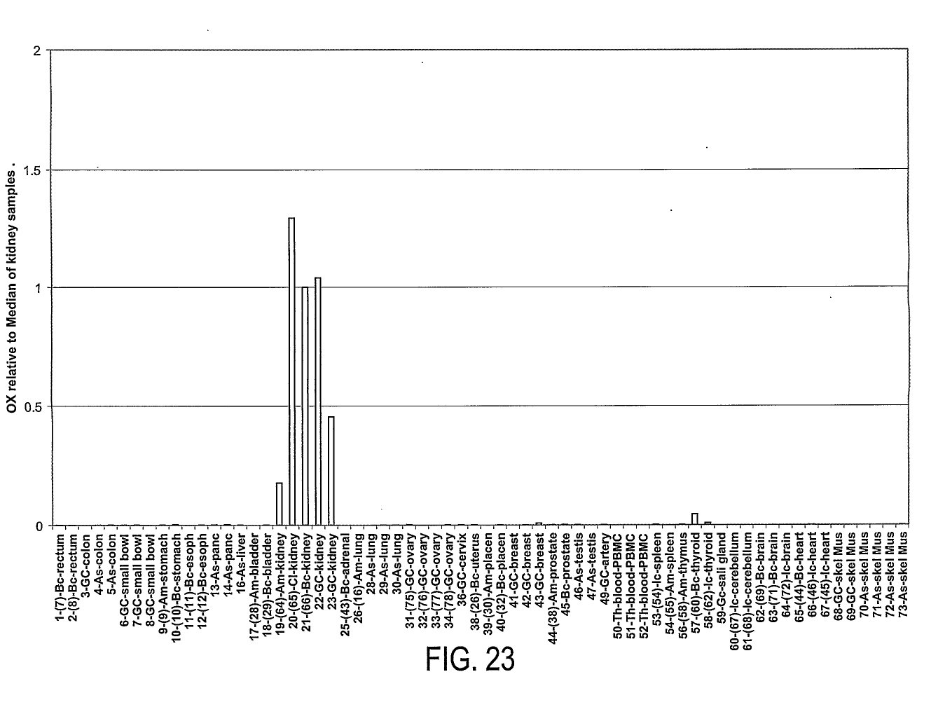

FIG. 23 is a histogram showing relative expression of the above-indicated Homo sapiens aldehyde reductase (aldose reductase) like 6 (ALDRL6) transcripts in kidney tissue samples as opposed to other tissues.

As is evident from FIG. 23, the expression of Homo sapiens aldehyde reductase (aldose reductase) like 6 (ALDRL6) transcripts detectable by the above amplicon in kidney tissue samples was significantly higher than in all the other samples.

Forward Primer (AA7030666_junc4-7F2)
(SEQ ID NO: 438):
CTTCCGGAACTACACGGTCC
Reverse Primer (AA7030666_junc4-7R2)
(SEQ ID NO: 439):
CTTGCTCCTGACGAAGTCCAC
Amplicon (AA7030666_junc4-7F2R2) (SEQ ID NO: 440):
CTTCCGGAACTACACGGTCCCCTCCTGGACCGTGTCTTCACCACCTACAA
GCTCATGCACACGCACCAGACAGTGGACTTCGTCAGGAGCAAG

### Description for Cluster AI590292

Cluster AI590292 features 3 transcript(s) and 10 segment(s) of interest, the names for which are given in Tables 74 and 75, respectively. The selected protein variants are given in table 76.

TABLE 74
Transcripts of interest
Transcript Name

AI590292_T5 (SEQ ID NO: 165)
AI590292_T6 (SEQ ID NO: 166)
AI590292_T7 (SEQ ID NO: 167)

TABLE 75
Segments of interest
Segment Name

AI590292_N2 (SEQ ID NO: 168)
AI590292_N10 (SEQ ID NO: 169)
AI590292_N16 (SEQ ID NO: 170)
AI590292_N17 (SEQ ID NO: 171)
AI590292_N0 (SEQ ID NO: 172)
AI590292_N1 (SEQ ID NO: 173)
AI590292_N6 (SEQ ID NO: 174)
AI590292_N8 (SEQ ID NO: 175)
AI590292_N12 (SEQ ID NO: 176)
AI590292_N14 (SEQ ID NO: 177)

TABLE 76
Proteins of interest

| Protein Name | Corresponding Transcript(s) |
|---|---|
| AI590292_P5 (SEQ ID NO: 180) | AI590292_T6 (SEQ ID NO: 166) |
| AI590292_P6 (SEQ ID NO: 181) | AI590292_T7 (SEQ ID NO: 167) |
| AI590292_P12 (SEQ ID NO: 182) | AI590292_T7 (SEQ ID NO: 167) |
| AI590292_P13 (SEQ ID NO: 183) | AI590292_T5 (SEQ ID NO: 165) |

These sequences are variants of the known protein Podocin (SEQ ID NO:178) (SwissProt accession identifier PODO_HUMAN (SEQ ID NO:178), referred to herein as the previously known protein.

Protein Podocin (SEQ ID NO:178) is known or believed to have the following function(s): Plays a role in the regulation of glomerular permeability, acting probably as a linker between the plasma membrane and the cytoskeleton. Known polymorphisms for this sequence are as shown in Table 77.

TABLE 77
Amino acid mutations for Known Protein

| SNP position(s) on | |
|---|---|
| amino acid sequence | Comment |
| 20 | P -> L (in SRN). /FTId = VAR_010231 |
| 92 | G -> C (in SRN). /FTId = VAR_010232 |
| 138 | R -> Q (in SRN). /FTId = VAR_010233 |
| 160 | D -> G (in SRN). /FTId = VAR_010234 |
| 180 | V -> M (in SRN). /FTId = VAR_010235 |
| 291 | R -> W (in SRN). /FTId = VAR_010236 |

Protein Podocin (SEQ ID NO:178) localization is believed to be Integral membrane protein.

The following GO Annotation(s) apply to the previously known protein. The following annotation(s) were found: excretion, which are annotation(s) related to Biological Process; protein binding, which are annotation(s) related to Molecular Function; and integral to plasma membrane, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

According to optional but preferred embodiments of the present invention, variants of this cluster according to the present invention (amino acid and/or nucleic acid sequences of AI590292) may optionally have one or more of the following utilities, as described in greater detail below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

A non-limiting example of such a utility is the detection, diagnosis and/or determination of steroid resistant nephrotic syndrome. The method comprises detecting a AI590292 variant, for example a variant protein, protein fragment, peptide, polynucleotide, polynucleotide fragment and/or oligonucleotide as described herein, optionally and preferably in a serum sample. The expression levels of the AI590292 variant as determined in a patient can be further compared to those in a normal individual.

Mutations of the known protein podocin were shown to cause steroid resistant nephrotic syndrome, as described with regard to US Patent Application No. 2003/0152954, hereby incorporated by reference as if fully set forth herein.

Another non-limiting example of such a utility is for monitoring, prognosing, diagnosing or treating renal disorders, e.g. renal diseases, injuries or toxicities. The method comprises detecting a AI590292 variant, for example a variant protein, protein fragment, peptide, polynucleotide, polynucleotide fragment and/or oligonucleotide as described herein, optionally and preferably in a serum sample. The expression levels of the AI590292 variant as determined in a patient can be further compared to those in a normal individual.

Differential expression of podocin was shown to be related to kidney disease and damage, as described with regard to US Patent Application No. 2006/0008804, hereby incorporated by reference as if fully set forth herein.

According to optional but preferred embodiments of the present invention, variants of this cluster according to the present invention (amino acid and/or nucleic acid sequences of AI590292) may optionally have one or more of the following utilities, as described with regard to Table 78 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 78
Table of Utilities for Variants of AI590292, related to podocin:

| Utility | Reason | Reference |
|---|---|---|
| mutations and genetic | any under expression of the | 10742096 |
| alteration for diagnosis of | gene/protein will be | |
| steroid-resistant nephrotic | manifested in any type of | |
| syndrome. | nephropathy | |
| under expression for the | any under expression of the | 15942677 |
| detection of focal | gene/protein will be | |
| segmental | manifested in any type of | |
| glomerulosclerosis | nephropathy | |
| under expression for the | any under expression of the | 15149332 |
| detection of proteinuria of | gene/protein will be | |
| patients with diabetes and | manifested in any type of | |
| nephropathy | nephropathy | |
| | | 11555666 |
| | | 11865096 |

According to other optional embodiments of the present invention, variants of this cluster according to the present invention (amino acid and/or nucleic acid sequences of AI590292) may optionally have one or more of the following utilities, some of which are related to utilities described above. It should be noted that these utilities are optionally and preferably suitable for human and non-human animals as subjects, except where otherwise noted.

Table 79 below describes diagnostic utilities for the cluster AI590292 that were found through microarrays, including the statistical significance thereof and a reference. One or more AI590292 variants according to the present invention may optionally have one or more of these utilities.

TABLE 79

| Utility | Microarray source |
|---|---|
| identification of kidney | jp_atlas, GNF1, GNF2 |
| cellular damage, due to very | |
| high expression in kidney. | |

As noted above, cluster AI590292 features 3 transcript(s), which were listed in Table 74 above. These transcript(s) encode for protein(s) which are variant(s) of protein Podocin (SEQ ID NO:178). A description of each variant protein according to the present invention is now provided.

Variant protein AI590292_P5 (SEQ ID NO:180) according to the present invention is encoded by transcript AI590292_T6 (SEQ ID NO:166). One or more alignments to one or more previously published protein sequences are given in the alignment table on the attached CD-ROM. A brief description of the relationship of the variant protein according to the present invention to each such aligned protein is as follows:

1. Comparison report between AI590292_P5 (SEQ ID NO:180) and PODO_HUMAN (SEQ ID NO:178):

A. An isolated chimeric polypeptide encoding for AI590292_P5 (SEQ ID NO:180), comprising a first amino acid sequence being at least 90% homologous to MERRARSSSRESRGRGGRTPHKENKRAKAERSGGGRGRQEAGPEPSGSGRAGTPGEPRA PAATVVDVDEVRGSGEEGTEVVALLESERPEE corresponding to amino acids 1-91 of PODO_HUMAN (SEQ ID NO:178), which also corresponds to amino acids 1-91 of AI590292_P5 (SEQ ID NO:180), and a second amino acid sequence being at least 90% homologous to GLFFFLPCLDTYHKVDLRLQTLEIPFHEIVTKDMFIMEIDAICYYRMENASLLLSSLAIIVS KAVQFLVQTTMKRLLAHRSLTEILLERKSIAQDAKVALDSVTCIWGIKVERIEIKDVRLPA GLQHSLAVEAEAQRQAKVRMIAAEAEKAASESLRMAAEILSGTPAAVQLRYLHTLQSLS TEKPSTVVLPLPFDLLNCLSSPSNRTQGSLPFPSPSKPVEPLNPKKKDSPML corresponding to amino acids 151-383 of PODO_HUMAN (SEQ ID NO:178), which also corresponds to amino acids 92-324 of AI590292_P5 (SEQ ID NO:180), wherein said first amino acid sequence and second amino acid sequence are contiguous and in a sequential order.

B. An isolated chimeric polypeptide encoding for an edge portion of AI590292_P5 (SEQ ID NO:180), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EG, having a structure as follows: a sequence starting from any of amino acid numbers 91-x to 91; and ending at any of amino acid numbers 92+((n−2)−x), in which x varies from 0 to n−2.

3. Comparison report between AI590292_P5 (SEQ ID NO:180) and Q9NP85-2 (SEQ ID NO:179):

A. An isolated chimeric polypeptide encoding for AI590292_P5 (SEQ ID NO:180), comprising a first amino acid sequence being at least 90% homologous to MERRARSSSRESRGRGGRTPHKENKRAKAERSGGGRGRQEAGPEPSGSGRAGTPGEPRA PAATVVDVDEVRGSGEEGTEVVALLESERPEE corresponding to amino acids 1-91 of Q9NP85-2 (SEQ ID NO:179), which also corresponds to amino acids 1-91 of AI590292_P5 (SEQ ID NO:180), a second amino acid sequence being at least 90% homologous to GLFFFLPCLDTYHKVDLRLQTLEIPFHE corresponding to amino acids 151-178 of Q9NP85-2 (SEQ ID NO:179), which also corresponds to amino acids 92-119 of AI590292_P5 (SEQ ID NO:180), a third amino acid sequence being at least 70%, optionally at least 80%, preferably at least 85%, more preferably at least 90% and most preferably at least 95% homologous to a polypeptide having the sequence IVTKDMFIMEIDAICYYRMENASLLLSSLAHVSKAVQFLVQTTMKRLLAHRSLTEILLER KSIAQDAK (SEQ ID NO:387) corresponding to amino acids 120-187 of AI590292_P5 (SEQ ID NO:180), and a fourth amino acid sequence being at least 90% homologous to VALDSVTCIWGIKVERIEIKDVRLPAGLQHSLAVEAEAQRQAKVRMIAAEAEKAASESLR MAAEILSGTPAAVQLRYLHTLQSLSTEKPSTVVLPLPFDLLNCLSSPSNRTQGSLPFPSPSK PVEPLNPKKKDSPML corresponding to amino acids 179-315 of Q9NP85-2 (SEQ ID NO:179), which also corresponds to amino acids 188-324 of AI590292_P5 (SEQ ID NO:180), wherein said first amino acid sequence, second amino acid sequence, third amino acid sequence and fourth amino acid sequence are contiguous and in a sequential order.

B. An isolated chimeric polypeptide encoding for an edge portion of AI590292 P5 (SEQ ID NO:180), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EG, having a structure as follows: a sequence starting from any of amino acid numbers 91-x to 91; and ending at any of amino acid numbers 92+((n−2)−x), in which x varies from 0 to n−2.

C. An isolated polypeptide encoding for an edge portion of AI590292_P5 (SEQ ID NO:180), comprising an amino acid sequence being at least 70%, optionally at least about 80%, preferably at least about 85%, more preferably at least about 90% and most preferably at least about 95% homologous to the sequence IVTKDMFIMEIDAICYYRMENASLLLSSLAHVSKAVQFLVQTTMKRLLAHRSLTEILLER KSIAQDAK (SEQ ID NO:387) of AI590292_P5 (SEQ ID NO:180).

The localization of the variant protein was determined according to results from a number of different software programs and analyses, including analyses from SignalP and other specialized programs. The variant protein is believed to be located intracellularly with regard to the cell.

Variant protein AI590292_P5 (SEQ ID NO:180) also has the following non-silent SNPs (Single Nucleotide Polymorphisms) as listed in Table 80, (given according to their position(s) on the amino acid sequence, with the alternative amino acid(s) listed).

TABLE 80
Amino acid mutations

| SNP position(s) on amino acid | |
|---|---|
| sequence | Alternative amino acid(s) |
| 89 | P -> |
| 137 | R -> * |
| 160 | Q -> P |
| 236 | A -> V |
| 250 | A -> V |
| 316 | P -> L |

Variant protein AI590292_P5 (SEQ ID NO:180) is encoded by the transcript AI590292_T6 (SEQ ID NO:166), for which the coding portion starts at position 89 and ends at position 1060. The transcript also has the following SNPs as listed in Table 81 (given according to their position on the nucleotide sequence, with the alternative nucleic acid listed).

TABLE 81
Nucleic acid SNPs

| SNP position(s) on nucleotide | |
|---|---|
| sequence | Alternative nucleic acid(s) |
| 38 | T -> G |
| 175 | C -> G |
| 190 | G -> A |
| 353 | C -> |
| 497 | C -> T |
| 567 | A -> C |
| 795 | C -> T |
| 802 | G -> A |
| 837 | C -> T |
| 865 | T -> C |
| 949 | A -> G |
| 1035 | C -> T |
| 1117 | C -> G |
| 1220 | A -> G |
| 1263 | G -> A |
| 1321 | A -> G |
| 1491 | G -> A |

Variant protein AI590292_P6 (SEQ ID NO:181) according to the present invention is encoded by transcript AI590292_T7 (SEQ ID NO:167). One or more alignments to one or more previously published protein sequences are given in the alignment table on the attached CD-ROM. A brief description of the relationship of the variant protein according to the present invention to each such aligned protein is as follows:

1. Comparison report between AI590292_P6 (SEQ ID NO:181) and PODO_HUMAN (SEQ ID NO:178):

A. An isolated chimeric polypeptide encoding for AI590292_P6 (SEQ ID NO:181), comprising a first amino acid sequence being at least 90% homologous to MERRARSSSRESRGRGGRTPHKENKRAKAERSGGGRGRQEAGPEPSGSGRAGTPGEPRA PAATVVDVDEVRGSGEEGTEVVALLESERPEE corresponding to amino acids 1-91 of PODO_HUMAN (SEQ ID NO:178), which also corresponds to amino acids 1-91 of AI590292_P6 (SEQ ID NO:181), a second amino acid sequence being at least 90% homologous GLFFFLPCLDTYHKVDLRLQTLEIPFHEIVTKDMFIMEIDAICYYRMENASLLLSSLAHVS KAVQFLVQTTMKRLLAHRSLTEILLERKSIAQDAKVALDSVTCIWGIKVERIEIKDVRLPA GLQHSLAVEAEAQRQAKVR corresponding to amino acids 151-291 of PODO_HUMAN (SEQ ID NO:178), which also corresponds to amino acids 92-232 of AI590292_P6 (SEQ ID NO:181), and a third amino acid sequence being at least 70%, optionally at least 80%, preferably at least 85%, more preferably at least 90% and most preferably at least 95% homologous to a polypeptide having the sequence LKTLQKEE (SEQ ID NO:388) corresponding to amino acids 233-240 of AI590292_P6 (SEQ ID NO:181), wherein said first amino acid sequence, second amino acid sequence and third amino acid sequence are contiguous and in a sequential order.

B. An isolated chimeric polypeptide encoding for an edge portion of AI590292_P6 (SEQ ID NO:181), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EG, having a structure as follows: a sequence starting from any of amino acid numbers 91-x to 91; and ending at any of amino acid numbers 92+((n−2)−x), in which x varies from 0 to n−2.

C. An isolated polypeptide encoding for an edge portion of AI590292_P6 (SEQ ID NO:181), comprising an amino acid sequence being at least 70%, optionally at least about 80%, preferably at least about 85%, more preferably at least about 90% and most preferably at least about 95% homologous to the sequence LKTLQKEE (SEQ ID NO:388) of AI590292_P6 (SEQ ID NO:181).

3. Comparison report between AI590292_P6 (SEQ ID NO:181) and Q9NP85-2 (SEQ ID NO:179):

A. An isolated chimeric polypeptide encoding for AI590292_P6 (SEQ ID NO:181), comprising a first amino acid sequence being at least 90% homologous to MERRARSSSRESRGRGGRTPHKENKRAKAERSGGGRGRQEAGPEPSGSGRAGTPGEPRA PAATVVDVDEVRGSGEEGTEVVALLESERPEE corresponding to amino acids 1-91 of Q9NP85-2 (SEQ ID NO:179), which also corresponds to amino acids 1-91 of AI590292_P6 (SEQ ID NO:181), a second amino acid sequence being at least 90% homologous to GLFFFLPCLDTYHKVDLRLQTLEIPFHE corresponding to amino acids 151-178 of Q9NP85-2 (SEQ ID NO:179), which also corresponds to amino acids 92-119 of AI590292_P6 (SEQ ID NO:181), a third amino acid sequence being at least 70%, optionally at least 80%, preferably at least 85%, more preferably at least 90% and most preferably at least 95% homologous to a polypeptide having the sequence IVTKDMFIMEIDAICYYRMENASLLLSSLAHVSKAVQFLVQTTMKRLLAHRSLTEILLER KSIAQDAK (SEQ ID NO:387) corresponding to amino acids 120-187 of AI590292_P6 (SEQ ID NO:181), a fourth amino acid sequence being at least 90% homologous to VALDSVTCIWGIKVERIEIKDVRLPAGLQHSLAVEAEAQRQAKVR corresponding to amino acids 179-223 of Q9NP85-2 (SEQ ID NO:179), which also corresponds to amino acids 188-232 of AI590292_P6 (SEQ ID NO:181), and a fifth amino acid sequence being at least 70%, optionally at least 80%, preferably at least 85%, more preferably at least 90% and most preferably at least 95% homologous to a polypeptide having the sequence LKTLQKEE (SEQ ID NO:388) corresponding to amino acids 233-240 of AI590292_P6 (SEQ ID NO:181), wherein said first amino acid sequence, second amino acid sequence, third amino acid sequence, fourth amino acid sequence and fifth amino acid sequence are contiguous and in a sequential order.

B. An isolated chimeric polypeptide encoding for an edge portion of AI590292_P6 (SEQ ID NO:181), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EG, having a structure as follows: a sequence starting from any of amino acid numbers 91-x to 91; and ending at any of amino acid numbers 92+((n−2)−x), in which x varies from 0 to n−2.

C. An isolated polypeptide encoding for an edge portion of AI590292_P6 (SEQ ID NO:181), comprising an amino acid sequence being at least 70%, optionally at least about 80%, preferably at least about 85%, more preferably at least about 90% and most preferably at least about 95% homologous to the sequence IVTKDMFIMEIDAICYYRMENASLLLSSLAHVSKAVQFLVQTTMKRLLAHRSLTEILLER KSIAQDAK (SEQ ID NO:387) of AI590292_P6 (SEQ ID NO:181).

D. An isolated polypeptide encoding for an edge portion of AI590292_P6 (SEQ ID NO:181), comprising an amino acid sequence being at least 70%, optionally at least about 80%, preferably at least about 85%, more preferably at least about 90% and most preferably at least about 95% homologous to the sequence LKTLQKEE (SEQ ID NO:388) of AI590292_P6 (SEQ ID NO:181).

The localization of the variant protein was determined according to results from a number of different software programs and analyses, including analyses from SignalP and other specialized programs. The variant protein is believed to be located intracellularly with regard to the cell.

Variant protein AI590292_P6 (SEQ ID NO:181) also has the following non-silent SNPs (Single Nucleotide Polymorphisms) as listed in Table 82, (given according to their position(s) on the amino acid sequence, with the alternative amino acid(s) listed).

TABLE 82
Amino acid mutations

| SNP position(s) on amino acid | |
|---|---|
| sequence | Alternative amino acid(s) |
| 89 | P -> |
| 137 | R -> * |
| 160 | Q -> P |

Variant protein AI590292_P6 (SEQ ID NO:181) is encoded by the transcript: AI590292_T7 (SEQ ID NO:167), for which the coding portion starts at position 89 and ends at position 808. The transcript also has the following SNPs as listed in Table 83 (given according to their position on the nucleotide sequence, with the alternative nucleic acid listed).

TABLE 83
Nucleic acid SNPs

| SNP position(s) on nucleotide | |
|---|---|
| sequence | Alternative nucleic acid(s) |
| 38 | T -> G |
| 175 | C -> G |
| 190 | G -> A |
| 353 | C -> |
| 497 | C -> T |
| 567 | A -> C |
| 814 | A -> G |
| 869 | G -> C |
| 953 | C -> T |
| 960 | G -> A |
| 995 | C -> T |
| 1023 | T -> C |
| 1107 | A -> G |
| 1193 | C -> T |
| 1275 | C -> G |
| 1378 | A -> G |
| 1421 | G -> A |
| 1479 | A -> G |
| 1649 | G -> A |

Variant protein AI590292_P12 (SEQ ID NO:182) according to the present invention is encoded by transcript AI590292_T7 (SEQ ID NO:167). One or more alignments to one or more previously published protein sequences are given in the alignment table on the attached CD-ROM. A brief description of the relationship of the variant protein according to the present invention to each such aligned protein is as follows:

1. Comparison report between AI590292_P12 (SEQ ID NO:182) and PODO_HUMAN (SEQ ID NO:178):

A. An isolated chimeric polypeptide encoding for AI590292_P12 (SEQ ID NO:182), comprising a first amino acid sequence being at least 70%, optionally at least 80%, preferably at least 85%, more preferably at least 90% and most preferably at least 95%, homologous to a polypeptide having the sequence ADPQRLHDCAPLPLALPRCCSSRPAALR (SEQ ID NO:390) corresponding to amino acids 1-29 of AI590292_P12 (SEQ ID NO:182), a second amino acid sequence being at least 90% homologous to MERRARSSSRESRGRGGRTPHKENKRAKAERSGGGRGRQEAGPEPSGSGRAGTPGEPRA PAATVVDVDEVRGSGEEGTEVVALLESERPEE corresponding to amino acids 1-91 of PODO_HUMAN (SEQ ID NO:178), which also corresponds to amino acids 30-120 of AI590292_P12 (SEQ ID NO:182), a third amino acid sequence being at least 90% homologous to GLFFFLPCLDTYHKVDLRLQTLEIPFHEIVTKDMFIMEIDAICYYRMENASLLLSSLAHVS KAVQFLVQTTMKRLLAHRSLTEILLERKSIAQDAKVALDSVTCIWGIKVERIEIKDVRLPA GLQHSLAVEAEAQRQAKVR corresponding to amino acids 151-291 of PODO_HUMAN (SEQ ID NO:178), which also corresponds to amino acids 121-261 of AI590292_P12 (SEQ ID NO:182), and a fourth amino acid sequence being at least 70%, optionally at least 80%, preferably at least 85%, more preferably at least 90% and most preferably at least 95% homologous to a polypeptide having the sequence LKTLQKEE (SEQ ID NO:388) corresponding to amino acids 262-269 of AI590292_P12 (SEQ ID NO:182), wherein said first amino acid sequence, second amino acid sequence, third amino acid sequence and fourth amino acid sequence are contiguous and in a sequential order.

B. An isolated polypeptide encoding for a head of AI590292_P12 (SEQ ID NO:182), comprising a polypeptide being at least 70%, optionally at least about 80%, preferably at least about 85%, more preferably at least about 90% and most preferably at least about 95% homologous to the sequence ADPQRLHDCAPLPLALPRCCSSRPAALR (SEQ ID NO:390) of AI590292_P12 (SEQ ID NO:182).

C. An isolated chimeric polypeptide encoding for an edge portion of AI590292_P12 (SEQ ID NO:182), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EG, having a structure as follows: a sequence starting from any of amino acid numbers 120-x to 120; and ending at any of amino acid numbers 121+((n−2)−x), in which x varies from 0 to n−2.

D. An isolated polypeptide encoding for an edge portion of AI590292_P12 (SEQ ID NO:182), comprising an amino acid sequence being at least 70%, optionally at least about 80%, preferably at least about 85%, more preferably at least about 90% and most preferably at least about 95% homologous to the sequence LKTLQKEE (SEQ ID NO:388) of AI590292_P12 (SEQ ID NO:182).

3. Comparison report between AI590292_P12 (SEQ ID NO:182) and Q9NP85-2 (SEQ ID NO:179):

A. An isolated chimeric polypeptide encoding for AI590292_P12 (SEQ ID NO:182), comprising a first amino acid sequence being at least 70%, optionally at least 80%, preferably at least 85%, more preferably at least 90% and most preferably at least 95%, homologous to a polypeptide having the sequence ADPQRLHRDCAPLPLALPRCCSSRPAALR (SEQ ID NO:390) corresponding to amino acids 1-29 of AI590292_P12 (SEQ ID NO:182), a second amino acid sequence being at least 90% homologous to MERRARSSSRESRGRGGRTPHKENKRAKAERSGGGRGRQEAGPEPSGSGRAGTPGEPRA PAATVVDVDEVRGSGEEGTEVVALILESERPEE corresponding to amino acids 1-91 of Q9NP85-2 (SEQ ID NO:179), which also corresponds to amino acids 30-120 of AI590292_P12 (SEQ ID NO:182), a third amino acid sequence being at least 90% homologous to GLFFFLPCLDTYHKVDLRLQTLEIPFHE corresponding to amino acids 151-178 of Q9NP85-2 (SEQ ID NO:179), which also corresponds to amino acids 121-148 of AI590292_P12 (SEQ ID NO:182), a fourth amino acid sequence being at least 70%, optionally at least 80%, preferably at least 85%, more preferably at least 90% and most preferably at least 95% homologous to a polypeptide having the sequence IVTKDMFIMEIDAICYYRMENASLLLSSLAHVSKAVQFLVQTTKRLLAHRSLTEILLER KSIAQDAK (SEQ ID NO:387) corresponding to amino acids 149-216 of AI590292_P12 (SEQ ID NO:182), a fifth amino acid sequence being at least 90% homologous to VALDSVTCIWGIKVERIEIKVRLPAGLQHSLAVEAEAQRQAKVR corresponding to amino acids 179-223 of Q9NP85-2 (SEQ ID NO:179), which also corresponds to amino acids 217-261 of AI590292_P12 (SEQ ID NO:182), and a sixth amino acid sequence being at least 70%, optionally at least 80%, preferably at least 85%, more preferably at least 90% and most preferably at least 95% homologous to a polypeptide having the sequence LKTLQKEE (SEQ ID NO:388) corresponding to amino acids 262-269 of AI590292_P12 (SEQ ID NO:182), wherein said first amino acid sequence, second amino acid sequence, third amino acid sequence, fourth amino acid sequence, fifth amino acid sequence and sixth amino acid sequence are contiguous and in a sequential order.

B. An isolated polypeptide encoding for a head of AI590292_P12 (SEQ ID NO:182), comprising a polypeptide being at least 70%, optionally at least about 80%, preferably at least about 85%, more preferably at least about 90% and most preferably at least about 95% homologous to the sequence ADPQRLHRDCAPLPLALPRCCSSRPAALR (SEQ ID NO:390) of AI590292_P12 (SEQ ID NO:182).

C. An isolated chimeric polypeptide encoding for an edge portion of AI590292_P12 (SEQ ID NO:182), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise EG, having a structure as follows: a sequence starting from any of amino acid numbers 120-x to 120; and ending at any of amino acid numbers 121+((n−2)−x), in which x varies from 0 to n−2.

D. An isolated polypeptide encoding for an edge portion of AI590292_P12 (SEQ ID NO:182), comprising an amino acid sequence being at least 70%, optionally at least about 80%, preferably at least about 85%, more preferably at least about 90% and most preferably at least about 95% homologous to the sequence IVTKDMFIMEIDAICYYRMENASLLLSSLAHVSKAVQFLVQTTMKRLLAHRSLTEILLER KSIAQDAK (SEQ ID NO:387) of AI590292_P12 (SEQ ID NO:182).

E. An isolated polypeptide encoding for an edge portion of AI590292_P12 (SEQ ID NO:182), comprising an amino acid sequence being at least 70%, optionally at least about 80%, preferably at least about 85%, more preferably at least about 90% and most preferably at least about 95% homologous to the sequence LKTLQKEE (SEQ ID NO:388) of AI590292_P12 (SEQ ID NO:182).

The localization of the variant protein was determined according to results from a number of different software programs and analyses, including analyses from SignalP and other specialized programs. The variant protein is believed to be located intracellularly with regard to the cell.

Variant protein AI590292_P12 (SEQ ID NO:182) also has the following non-silent SNPs (Single Nucleotide Polymorphisms) as listed in Table 84, (given according to their position(s) on the amino acid sequence, with the alternative amino acid(s) listed).

TABLE 84
Amino acid mutations

| SNP position(s) on amino acid | |
|---|---|
| sequence | Alternative amino acid(s) |
| 13 | L -> V |
| 118 | P -> |
| 166 | R -> * |
| 189 | Q -> P |

Variant protein AI590292P112 (SEQ ID NO:182) is encoded by the transcript AI590292_T7 (SEQ ID NO:167), for which the coding portion starts at position 2 and ends at position 808. The transcript also has the following SNPs as listed in Table 85 (given according to their position on the nucleotide sequence, with the alternative nucleic acid listed).

TABLE 85
Nucleic acid SNPs

| SNP position(s) on nucleotide | |
|---|---|
| sequence | Alternative nucleic acid(s) |
| 38 | T -> G |
| 175 | C -> G |
| 190 | G -> A |
| 353 | C -> |
| 497 | C -> T |
| 567 | A -> C |
| 814 | A -> G |
| 869 | G -> C |
| 953 | C -> T |
| 960 | G -> A |
| 995 | C -> T |
| 1023 | T -> C |
| 1107 | A -> G |
| 1193 | C -> T |
| 1275 | C -> G |
| 1378 | A -> G |
| 1421 | G -> A |
| 1479 | A -> G |
| 1649 | G -> A |

Variant protein AI590292_P13 (SEQ ID NO:183) according to the present invention is encoded by transcript AI590292_T5 (SEQ ID NO:165). One or more alignments to one or more previously published protein sequences are given in the alignment table on the attached CD-ROM. A brief description of the relationship of the variant protein according to the present invention to each such aligned protein is as follows:

1. Comparison report between AI590292_P13 (SEQ ID NO:183) and PODO_HUMAN (SEQ ID NO:178):

A. An isolated chimeric polypeptide encoding for AI590292_P13 (SEQ ID NO:183), comprising a first amino acid sequence being at least 90% homologous to MERRARSSSRESRGRGGRTPHKENKRAKAERSGGGRGRQEAGPEPSGSGRAGTPGEPRA PAATWDVDEVRGSGEEGTEVVALLESERPEEG corresponding to amino acids 1-92 of PODO_HUMAN (SEQ ID NO:178), which also corresponds to amino acids 1-92 of AI590292_P13 (SEQ ID NO:183), and a second amino acid sequence being at least 70%, optionally at least 80%, preferably at least 85%, more preferably at least 90% and most preferably at least 95% homologous to a polypeptide having the sequence CTRV (SEQ ID NO:393) corresponding to amino acids 93-96 of AI590292_P13 (SEQ ID NO:183), wherein said first amino acid sequence and second amino acid sequence are contiguous and in a sequential order.

B. An isolated polypeptide encoding for an edge portion of AI590292_P13 (SEQ ID NO:183), comprising an amino acid sequence being at least 70%, optionally at least about 80%, preferably at least about 85%, more preferably at least about 90% and most preferably at least about 95% homologous to the sequence CTRV (SEQ ID NO:393) of AI590292_P13 (SEQ ID NO:183).

The localization of the variant protein was determined according to results from a number of different software programs and analyses, including analyses from SignalP and other specialized programs. The variant protein is believed to be located intracellularly with regard to the cell.

Variant protein AI590292_P13 (SEQ ID NO:183) also has the following non-silent SNPs (Single Nucleotide Polymorphisms) as listed in Table 86, (given according to their position(s) on the amino acid sequence, with the alternative amino acid(s) listed).

TABLE 86
Amino acid mutations

| SNP position(s) on amino acid | |
|---|---|
| sequence | Alternative amino acid(s) |
| 89 | P -> |

Variant protein AI590292_P13 (SEQ ID NO:183) is encoded by the transcript AI590292_T5 (SEQ ID NO:165), for which the coding portion starts at position 89 and ends at position 376. The transcript also has the following SNPs as listed in Table 87 (given according to their position on the nucleotide sequence, with the alternative nucleic acid listed).

TABLE 87
Nucleic acid SNPs

| SNP position(s) on nucleotide | Alternative |
|---|---|
| sequence | nucleic acid(s) |
| 38 | T -> G |
| 175 | C -> G |
| 190 | G -> A |
| 353 | C -> |
| 409 | T -> C |
| 570 | C -> T |
| 640 | A -> C |
| 868 | C -> T |
| 875 | G -> A |
| 910 | C -> T |
| 938 | T -> C |
| 1022 | A -> G |
| 1108 | C -> T |
| 1190 | C -> G |
| 1293 | A -> G |
| 1336 | G -> A |
| 1394 | A -> G |
| 1564 | G -> A |

As noted above, cluster AI590292 features 10 segments, which were listed in Table 75. These segments are portions of nucleic acid sequences which are described herein separately because they are of particular interest. A description of some of these segment according to the present invention is now provided.

Segment cluster AI590292_N2 (SEQ ID NO:168) according to the present invention is supported by 6 libraries. The number of libraries was determined as previously described. This segment can be found in the following transcript(s): AI590292_T5 (SEQ ID NO:165), AI590292_T6 (SEQ ID NO:166) and AI590292_T7 (SEQ ID NO:167). Table 88 below describes the starting and ending position of this segment on each transcript.

TABLE 88
Segment location on transcripts

| | Segment | Segment |
|---|---|---|
| Transcript name | starting position | ending position |
| AI590292_T5 (SEQ ID NO: 165) | 145 | 362 |
| AI590292_T6 (SEQ ID NO: 166) | 145 | 362 |
| AI590292_T7 (SEQ ID NO: 167) | 145 | 362 |

Segment cluster AI590292N116 (SEQ ID NO:170) according to the present invention is supported by 1 libraries. The number of libraries was determined as previously described. This segment can be found in the following transcript(s): AI590292_T7 (SEQ ID NO:167). Table 89 below describes the starting and ending position of this segment on each transcript.

TABLE 89
Segment location on transcripts

| | Segment | Segment |
|---|---|---|
| Transcript name | starting position | ending position |
| AI590292_T7 (SEQ ID NO: 167) | 785 | 942 |

According to an optional embodiment of the present invention, short segments related to the above cluster are also provided. These segments are up to about 120 bp in length, and so are included in a separate description.

Segment cluster AI590292 N6 (SEQ ID NO:174) according to the present invention is supported by 6 libraries. The number of libraries was determined as previously described. This segment can be found in the following transcript(s): AI590292_T5 (SEQ ID NO:165). Table 90 below describes the starting and ending position of this segment on each transcript.

TABLE 90
Segment location on transcripts

| | Segment | Segment |
|---|---|---|
| Transcript name | starting position | ending position |
| AI590292_T5 (SEQ ID NO: 165) | 363 | 435 |

Segment cluster AI590292_N8 (SEQ ID NO:175) according to the present invention is supported by 6 libraries. The number of libraries was determined as previously described. This segment can be found in the following transcript(s): AI590292_T5 (SEQ ID NO:165), AI590292_T6 (SEQ ID NO:166) and AI590292_T7 (SEQ ID NO:167). Table 91 below describes the starting and ending position of this segment on each transcript.

TABLE 91
Segment location on transcripts

| | Segment | Segment |
|---|---|---|
| Transcript name | starting position | ending position |
| AI590292_T5 (SEQ ID NO: 165) | 436 | 518 |
| AI590292_T6 (SEQ ID NO: 166) | 363 | 445 |
| AI590292_T7 (SEQ ID NO: 167) | 363 | 445 |

Expression of Homo sapiens nephrosis 2, idiopathic, steroid-resistant (podocin) (NPHS2) AI590292 transcripts which are detectable by amplicon as depicted in sequence name AI590292junc2-6 (SEQ ID NO:186) specifically in kidney tissue

Expression of Homo sapiens nephrosis 2, idiopathic, steroid-resistant (podocin) (NPHS2) transcripts detectable by or according to junc2-6—AI590292_junc2-6 (SEQ ID NO:186) amplicon and primers AI590292_junc2-6F (SEQ ID NO:184) and AI590292_junc2-6R (SEQ ID NO:185) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

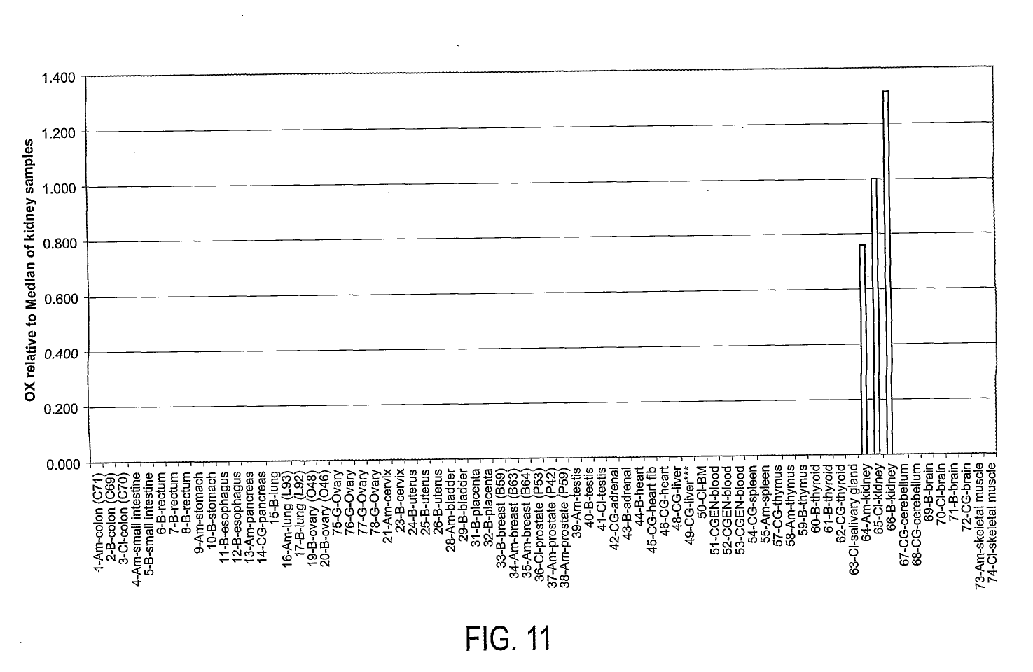

FIG. 11 is a histogram showing relative expression of the above-indicated Homo sapiens nephrosis 2, idiopathic, steroid-resistant (podocin) (NPHS2) transcripts in kidney tissue samples as opposed to other tissues.

As is evident from FIG. 11, the expression of Homo sapiens nephrosis 2, idiopathic, steroid-resistant (podocin) (NPHS2) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: AI590292_junc2-6F (SEQ ID NO:184) forward primer; and AI590292_junc2-6R (SEQ ID NO:185) reverse primer.

The present invention also preferably encompasses any amplicon obtained through the use of any suitable primer pair; for example, for the above experiment, the following amplicon was obtained as a non-limiting illustrative example only of a suitable amplicon: AI590292_junc2-6 (SEQ ID NO:186).

Forward Primer (AI590292_junc2-6F
(SEQ ID NO: 184):
GGACGTGGATGAGGTCCG
Reverse Primer (AI590292_junc2-6R
(SEQ ID NO: 185):
CTCTTTCATACTCTTGTACAACCTTCC
Amplicon (AI590292_junc2-6 (SEQ ID NO: 186):
GGACGTGGATGAGGTCCGAGGCTCCGGCGAGGAGGGCACCGAGGTGGTGG
CGCTGTTGGAGAGCGAGCGGCCCGAGGAAGGTTGTACAAGAGTATGAAAG
AG

Expression of Homo sapiens nephrosis 2, idiopathic, steroid-resistant (podocin) (NPHS2) AI590292 transcripts which are detectable by amplicon as depicted in sequence name AI590292_seg4WT (SEQ ID NO:189) specifically in kidney tissue

Expression of Homo sapiens nephrosis 2, idiopathic, steroid-resistant (podocin) (NPHS2) transcripts detectable by or according to seg4WT—AI590292_seg4WT (SEQ ID NO:189) amplicon and primers AI590292_seg4WTF (SEQ ID NO:187) and AI590292_seg4WTR (SEQ ID NO:188) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

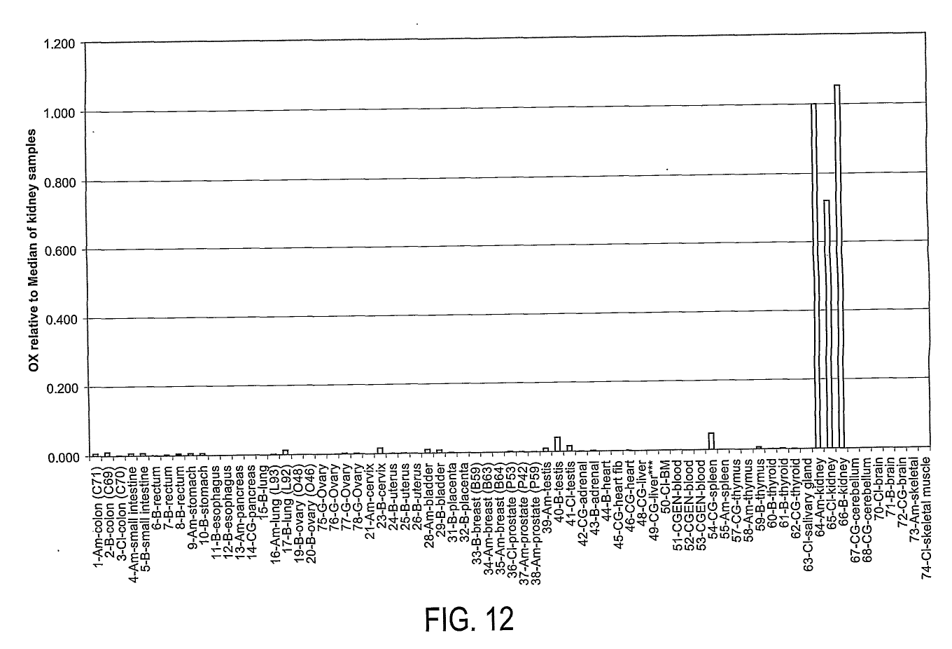

FIG. 12 is a histogram showing relative expression of the above-indicated Homo sapiens nephrosis 2, idiopathic, steroid-resistant (podocin) (NPHS2) transcripts in kidney tissue samples as opposed to other tissues.

As is evident from FIG. 12, the expression of Homo sapiens nephrosis 2, idiopathic, steroid-resistant (podocin) (NPHS2) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: AI590292_seg4WTF (SEQ ID NO:187) forward primer; and AI590292_seg4WTR (SEQ ID NO:188) reverse primer.

The present invention also preferably encompasses any amplicon obtained through the use of any suitable primer pair; for example, for the above experiment, the following amplicon was obtained as a non-limiting illustrative example only of a suitable amplicon: AI590292_seg4WT (SEQ ID NO:189).

Forward Primer (AI590292_seg4WTF (SEQ ID NO: 187):
GTACCAAATCCTCCGGCTTAG
Reverse Primer (AI590292_seg4WTR (SEQ ID NO: 188):
CTTTACGCAGAACCAGATGGA
Amplicon (AI590292_seg4WT (SEQ ID NO: 189):
GTACCAAATCCTCCGGCTTAGGGGCCTGTGAGTGGCTTCTTGTCCTCATT
TCCCTGCTCTTCATCATCATGACCTTCCCTTTTTCCATCTGGTTCTGCGT
AAAG

### Description for Cluster HUMUMOD

Cluster HUMUMOD features 4 transcript(s) and 49 segment(s) of interest, the names for which are given in Tables 92 and 93, respectively. The selected protein variants are given in table 94.

TABLE 92
Transcripts of interest
Transcript Name

HUMUMOD_T11 (SEQ ID NO: 190)
HUMUMOD_T13 (SEQ ID NO: 191)
HUMUMOD_T20 (SEQ ID NO: 192)
HUMUMOD_T31 (SEQ ID NO: 193)

TABLE 93
Segments of interest
Segment Name

HUMUMOD_N7 (SEQ ID NO: 194)
HUMUMOD_N33 (SEQ ID NO: 195)
HUMUMOD_N55 (SEQ ID NO: 196)
HUMUMOD_N56 (SEQ ID NO: 197)
HUMUMOD_N58 (SEQ ID NO: 198)
HUMUMOD_N61 (SEQ ID NO: 199)
HUMUMOD_N64 (SEQ ID NO: 200)
HUMUMOD_N72 (SEQ ID NO: 201)
HUMUMOD_N0 (SEQ ID NO: 202)
HUMUMOD_N11 (SEQ ID NO: 203)
HUMUMOD_N12 (SEQ ID NO: 204)
HUMUMOD_N13 (SEQ ID NO: 205)
HUMUMOD_N14 (SEQ ID NO: 206)
HUMUMOD_N15 (SEQ ID NO: 207)
HUMUMOD_N16 (SEQ ID NO: 208)
HUMUMOD_N17 (SEQ ID NO: 209)
HUMUMOD_N18 (SEQ ID NO: 210)
HUMUMOD_N19 (SEQ ID NO: 211)
HUMUMOD_N20 (SEQ ID NO: 212)
HUMUMOD_N21 (SEQ ID NO: 213)
HUMUMOD_N22 (SEQ ID NO: 214)
HUMUMOD_N23 (SEQ ID NO: 215)
HUMUMOD_N24 (SEQ ID NO: 216)
HUMUMOD_N25 (SEQ ID NO: 79)
HUMUMOD_N26 (SEQ ID NO: 217)
HUMUMOD_N27 (SEQ ID NO: 218)
HUMUMOD_N28 (SEQ ID NO: 219)
HUMUMOD_N29 (SEQ ID NO: 220)
HUMUMOD_N30 (SEQ ID NO: 221)
HUMUMOD_N31 (SEQ ID NO: 222)
HUMUMOD_N32 (SEQ ID NO: 223)
HUMUMOD_N34 (SEQ ID NO: 224)
HUMUMOD_N35 (SEQ ID NO: 225)
HUMUMOD_N36 (SEQ ID NO: 226)
HUMUMOD_N37 (SEQ ID NO: 227)
HUMUMOD_N38 (SEQ ID NO: 228)
HUMUMOD_N39 (SEQ ID NO: 229)
HUMUMOD_N40 (SEQ ID NO: 230)
HUMUMOD_N41 (SEQ ID NO: 231)
HUMUMOD_N44 (SEQ ID NO: 232)
HUMUMOD_N47 (SEQ ID NO: 233)
HUMUMOD_N48 (SEQ ID NO: 234)
HUMUMOD_N50 (SEQ ID NO: 235)
HUMUMOD_N51 (SEQ ID NO: 236)
HUMUMOD_N52 (SEQ ID NO: 237)
HUMUMOD_N53 (SEQ ID NO: 238)
HUMUMOD_N65 (SEQ ID NO: 239)
HUMUMOD_N67 (SEQ ID NO: 240)
HUMUMOD_N70 (SEQ ID NO: 241)

TABLE 94
Proteins of interest

| | Corresponding |
|---|---|
| Protein Name | Transcript(s) |
| HUMUMOD_P6 (SEQ ID NO: 246) | HUMUMOD_T11 |
| | (SEQ ID NO: 190) |
| HUMUMOD_P7 (SEQ ID NO: 247) | HUMUMOD_T13 |
| | (SEQ ID NO: 191) |
| HUMUMOD_P14 (SEQ ID NO: 248) | HUMUMOD_T20 |
| | (SEQ ID NO: 192) |
| HUMUMOD_P24 (SEQ ID NO: 249) | HUMUMOD_T31 |
| | (SEQ ID NO: 193) |

These sequences are variants of the known protein Uromodulin precursor (SEQ ID NO:242) (SwissProt accession identifier UROM_HUMAN (SEQ ID NO:242); known also according to the synonyms Tamm-Horsfall urinary glycoprotein; THP), referred to herein as the previously known protein.

Protein Uromodulin precursor (SEQ ID NO:242) is known or believed to have the following function(s): Not known. May play a role in regulating the circulating activity of cytokines as it binds to IL-1, IL-2 and TNF with high affinity. Known polymorphisms for this sequence are as shown in Table 95.

TABLE 95
Amino acid mutations for Known Protein

| SNP position(s) on | |
|---|---|
| amino acid sequence | Comment |
| 103 | G -> C (in MCKD2). /FTId = VAR_017666 |
| 148 | C -> Y (in HNFJ). /FTId = VAR_017667 |
| 217 | C -> R (in HNFJ). /FTId = VAR_017668 |
| 565 | H -> D |

Protein Uromodulin precursor (SEQ ID NO:242) localization is believed to be Attached to the membrane by a GPI-anchor, then cleaved to produce a soluble form which is secreted in urine.

The following GO Annotation(s) apply to the previously known protein. The following annotation(s) were found: cellular defense response; negative regulation of cell proliferation, which are annotation(s) related to Biological Process; and extrinsic to membrane, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

The variants HUMUMOD_P14 (SEQ ID NO:248) and HUMUMOD_P24 (SEQ ID NO:249) were previously disclosed by the inventors in published PCT application no WO2005/071058 and U.S. application Ser. Nos. 11/043,860 and 10/873,314, hereby incorporated by reference as if fully set forth herein, but have now been shown to have novel and surprising diagnostic uses as described herein for other variants of cluster HUMUMOD.

According to optional but preferred embodiments of the present invention, variants of this cluster according to the present invention (amino acid and/or nucleic acid sequences of HUMUMOD) may optionally have one or more of the following utilities, as described in greater detail below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

A non-limiting example of such a utility is the detection, diagnosis and/or determination of early stage of renal disease and/or renal complications of a disease, particularly diabetes (diabetic nephropathy) but also including any one of: nephropathy, diabetes insipidus, diabetes type I, diabetes II, renal disease (glomerulonephritis, bacterial and viral glomerulonephritides, IgA nephropathy and Henoch-Schenlein Purpura, membranoproliferative glomerulonephritis, membranous nephropathy, Sjogren's syndrome, nephrotic syndrome (minimal change disease, focal glomerulosclerosis and related disorders), acute renal failure, acute tubulointerstitial nephritis, pyelonephritis, GU tract inflammatory disease, Pre-clampsia, renal graft rejection, leprosy, reflux nephropathy, nephrolithiasis), genetic renal disease (medullary cystic, medullar sponge, polycystic kidney disease (autosomal dominant polycystic kidney disease, autosomal recessive polycystic kidney disease, tuborous sclerosis), von Hippel-Lindau disease, familial thin-glomerular basement membrane disease, collagen III glomerulopathy, fibronectin glomerulopathy, Alport's syndrome, Fabry's disease, Nail-Patella Syndrome, congenital urologic anomalies), monoclonal gammopathies (multiple myeloma, amyloidosis and related disorders), febrile illness (familial Mediterranean fever, HIV infection-AIDS), inflammatory disease (systemic vasculitides (polyarteritis nodosa, Wegener's granulomatosis, polyarteritis, necrotizing and crescentic glomerulonephritis), polymyositis-dermatomyositis, pancreatitis, rheumatoid arthritis, systemic lupus erythematosus, gout), blood disorders (sickle cell disease, thrombotic thrombocytopenia purpura, hemolytic-uremic syndrome, acute cortical necrosis, renal thromboembolism), trauma and surgery (extensive injury, bums, abdominal and vascular surgery, induction of anesthesia), drugs (penicillamine, steroids) and drug abuse, malignant disease (epithelial (lung, breast), adenocarcinoma (renal), melanoma, lymphoreticular, multiple myeloma), circulatory disease (myocardial infarction, cardiac failure, peripheral vascular disease, hypertension, coronary heart disease, non-atherosclerotic cardiovascular disease, atherosclerotic cardiovascular disease), skin disease (psoriasis, systemic sclerosis), respiratory disease (COPD, obstructive sleep apnoea, hypoia at high altitude) and endocrine disease (acromegaly, diabetes mellitus, diabetes insipidus). The method comprises detecting a HUMUMOD variant, for example a variant protein, protein fragment, peptide, polynucleotide, polynucleotide fragment and/or oligonucleotide as described herein, optionally and preferably in a serum sample. The expression levels of the HUMUMOD variant as determined in a patient can be further compared to those in a normal individual.

Differential expression of the known Uromodulin for the above utilities is described with regard to US Patent Application No. 2004/0029175, hereby incorporated by reference as if fully set forth herein.

Another non-limiting example of such a utility is the detection, diagnosis and/or determination of diseases of the kidney including, but not limited to, Bartter's syndrome, Gitelman syndrome, nephrolithiasis, renal amyloidosis, hypertension; primary aldosteronism; Addison's disease; renal failure; glomerulonephritis; chronic glomerulonephritis: tubulointerstitial nephritis; cystic disorders of the kidney and dysplastic malformations such as polycystic disease, renal dysplasias, and cortical or medullary cysts; inherited polycystic renal diseases (PRD), such as recessive and autosomal dominant PRD; medullary cystic disease; medullary sponge kidney and tubular dysplasia; Alport's syndrome; non-renal cancers which affect renal physiology, such as bronchogenic tumors of the lungs or tumors of the basal region of the brain; multiple myeloma; adenocarcinomas of the kidney; metastatic renal carcinoma; in addition, nephrotoxic disorders include any functional or morphologic change in the kidney produced by any pharmaceutical, chemical, or biological agent that is ingested, injected, inhaled, or absorbed. The method comprises detecting a HUMUMOD variant, for example a variant protein, protein fragment, peptide, polynucleotide, polynucleotide fragment and/or oligonucleotide as described herein, optionally and preferably in a serum sample. The expression levels of the HUMUMOD variant as determined in a patient can be further compared to those in a normal individual.

Differential expression of the known Uromodulin for the above utilities is described with regard to U.S. Pat. No. 6,277,574, hereby incorporated by reference as if fully set forth herein.

Another non-limiting example of such a utility is the detection, diagnosis and/or determination of renal diseases or a predisposition to renal diseases, including but not limited to renal failure, hyperuricemia, gouty arthritis or enuresis, familial juvenile gouty nephropathy, Medullary cystic kidney disease 2. The method comprises detecting a HUMUMOD variant, for example a variant protein, protein fragment, peptide, polynucleotide, polynucleotide fragment and/or oligonucleotide as described herein, optionally and preferably in a serum sample. The expression levels of the HUMUMOD variant as determined in a patient can be further compared to those in a normal individual.

Differential expression of the known Uromodulin for the above utilities is described with regard to PCT Application No. WO 04/038377, hereby incorporated by reference as if fully set forth herein.

Another non-limiting example of such a utility is the detection, diagnosis and/or determination of multiple sclerosis. The method comprises detecting a HUMUMOD variant, for example a variant protein, protein fragment, peptide, polynucleotide, polynucleotide fragment and/or oligonucleotide as described herein, optionally and preferably in a serum sample. The expression levels of the HUMUMOD variant as determined in a patient can be further compared to those in a normal individual.

Differential expression of the known Uromodulin for the above utility is described with regard to PCT Application No. WO 04/028339, hereby incorporated by reference as if fully set forth herein.

According to optional but preferred embodiments of the present invention, variants of this cluster according to the present invention (amino acid and/or nucleic acid sequences of HUMUMOD) may optionally have one or more of the following utilities, as described with regard to Table 96 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 96
Table of Utilities for Variants of HUMUMOD, related to Uromodulin:

| Utility | Reason | Reference |
|---|---|---|
| mutation for diagnosis of | Mutations of the UMOD | 12471200 |
| kidney disease | gene are responsible for | |
| | medullary cystic kidney | |
| | disease 2 and familial | |
| | juvenile hyperuricaemic | |
| | nephropathy | |

According to other optional embodiments of the present invention, variants of this cluster according to the present invention (amino acid and/or nucleic acid sequences of HUMUMOD) may optionally have one or more of the following utilities, some of which are related to utilities described above. It should be noted that these utilities are optionally and preferably suitable for human and non-human animals as subjects, except where otherwise noted.

Table 97 below describes diagnostic utilities for the cluster HUMUMOD that were found through microarrays, including the statistical significance thereof and a reference. One or more HUMUMOD variants according to the present invention may optionally have one or more of these utilities.

TABLE 97

| Utility | Microarray source |
|---|---|
| identification of kidney | jp_atlas, GNF1, GNF2, |
| cellular damage, due to very | med_all_avg (internal |
| high expression in kidney. | database). |

As noted above, cluster HUMUMOD features 4 transcript(s), which were listed in Table 92 above. These transcript(s) encode for protein(s) which are variant(s) of protein Uromodulin precursor (SEQ ID NO:242). A description of each variant protein according to the present invention is now provided.

Variant protein HUMUMOD_P6 (SEQ ID NO:246) according to the present invention is encoded by transcript HUMUMOD_T11 (SEQ ID NO:190). One or more alignments to one or more previously published protein sequences are given in the alignment table on the attached CD-ROM. A brief description of the relationship of the variant protein according to the present invention to each such aligned protein is as follows:

1. Comparison report between HUMUMOD_P6 (SEQ ID NO:246) and UROM_HUMAN (SEQ ID NO:242):

A. An isolated chimeric polypeptide encoding for HUMUMOD_P6 (SEQ ID NO:246), comprising a first amino acid sequence being at least 90% homologous to MGQPSLTWMLMVVVASWFITTAATDTSEARWCSECHSNATCTEDEAVTTCTCQEGFTG DGLTCVDLDECAIPGAHNCSANSSCVNTPGSFSCVCPEGFRLSPGLGCTDVDECAEPGLS HCHALATCVNVVGSYLCVCPAGYRGDGWHCECSPGSCGPGLDCVPEGDALVCADPCQ AHRTLDEYWRSTEYGEGYACDTDLRGWYRFVGQGGARMAETCVPVLRCNTAAPMWL NGTHPSSDEGIVSRKACAHWSGHCCLWDASVQVKACAGGYYVYNLTAPPECHLAYCT DPSSVEGTCEECSIDEDCKSNNGRWHCQCKQDFNIT corresponding to amino acids 1-324 of UROM_HUMAN (SEQ ID NO:242), which also corresponds to amino acids 1-324 of HUMUMOD_P6 (SEQ ID NO:246), and a second amino acid sequence being at least 70%, optionally at least 80%, preferably at least 85%, more preferably at least 90% and most preferably at least 95% homologous to a polypeptide having the sequence APEHKPGPVSSPPISSGLMTSPPISSVLRARHKPQISHRYLPPGAQAGMWGQ (SEQ ID NO:394) corresponding to amino acids 325-376 of HUMUMOD_P6 (SEQ ID NO:246), wherein said first amino acid sequence and second amino acid sequence are contiguous and in a sequential order.

B. An isolated polypeptide encoding for an edge portion of HUMUMOD_P6 (SEQ ID NO:246), comprising an amino acid sequence being at least 70%, optionally at least about 80%, preferably at least about 85%, more preferably at least about 90% and most preferably at least about 95% homologous to the sequence APEHKPGPVSSPPISSGLMTSPPISSVLRARHKPQISHRYLPPGAQAGMWGQ (SEQ ID NO:394) of HUMUMOD_P6 (SEQ ID NO:246).

4. Comparison report between HUMUMOD_P6 (SEQ ID NO:246) and Q8IYG0_HUMAN (SEQ ID NO:244):

A. An isolated chimeric polypeptide encoding for HUMUMOD_P6 (SEQ ID NO:246), comprising a first amino acid sequence being at least 90% homologous to MGQPSLTWMLMVVVASWFITTAATDTSEARWCSECHSNATCTEDEAVTTCTCQEGFTG DGLTCVDLDECAIPGAHNCSANSSCVNTPGSFSCVCPEGFRLSPGLGCTDVDECAEPGLS HCHALATCVNVVGSYLCVCPAGYRGDGWHCECSPGSCGPGLDCVPEGDALVCADPCQ AHRTLDEYWRSTEYGEGYACDTDLRGWYR corresponding to amino acids 1-204 of Q8IYG0_HUMAN (SEQ ID NO:244), which also corresponds to amino acids 1-204 of HUMUMOD_P6 (SEQ ID NO:246), a second amino acid sequence being at least 70%, optionally at least 80%, preferably at least 85%, more preferably at least 90% and most preferably at least 95% homologous to a polypeptide having the sequence FVGQGGARMAETCVPVLRCNTAAPMWLNGT (SEQ ID NO:395) corresponding to amino acids 205-234 of HUMUMOD_P6 (SEQ ID NO:246), a third amino acid sequence being at least 90% homologous to HPSSDEGIVSRKACAHWSGHCCLWDASVQVKACAGGYYVYNLTAPPECHLAYCTDPSS VEGTCEECSIDEDCKSNNGRWHCQCKQDFNIT corresponding to amino acids 206-295 of Q8IYG0_HUMAN (SEQ ID NO:244), which also corresponds to amino acids 235-324 of HUMUMOD_P6 (SEQ ID NO:246), and a fourth amino acid sequence being at least 70%, optionally at least 80%, preferably at least 85%, more preferably at least 90% and most preferably at least 95% homologous to a polypeptide having the sequence APEHKPGPVSSPPISSGLMTSPPISSVLRARHKPQISHRYLPPGAQAGMWGQ (SEQ ID NO:394) corresponding to amino acids 325-376 of HUMUMOD_P6 (SEQ ID NO:246), wherein said first amino acid sequence, second amino acid sequence, third amino acid sequence and fourth amino acid sequence are contiguous and in a sequential order.

B. An isolated polypeptide encoding for an edge portion of HUMUMOD_P6 (SEQ ID NO:246), comprising an amino acid sequence being at least 70%, optionally at least about 80%, preferably at least about 85%, more preferably at least about 90% and most preferably at least about 95% homologous to the sequence FVGQGGARMAETCVPVLRCNTAAPMWLNGT (SEQ ID NO:395) of HUMUMOD_P6 (SEQ ID NO:246).

C. An isolated polypeptide encoding for an edge portion of HUMUMOD_P6 (SEQ ID NO:246), comprising an amino acid sequence being at least 70%, optionally at least about 80%, preferably at least about 85%, more preferably at least about 90% and most preferably at least about 95% homologous to the sequence APEHKPGPVSSPPISSGLMTSPPISSVLRARHKPQISHRYLPPGAQAGMWGQ (SEQ ID NO:394) of HUMUMOD_P6 (SEQ ID NO:246).

5. Comparison report between HUMUMOD_P6 (SEQ ID NO:246) and Q6ZS84_HUMAN (SEQ ID NO:245):

A. An isolated chimeric polypeptide encoding for HUMUMOD_P6 (SEQ ID NO:246), comprising a first amino acid sequence being at least 90% homologous to MGQPSLTWMLMVVVASWFITTAATDTSEARWCSECHSNATCTEDEAVTTCTCQEGFTG DGLTCVD corresponding to amino acids 1-65 of Q6ZS84_HUMAN (SEQ ID NO:245), which also corresponds to amino acids 1-65 of HUMUMOD_P6 (SEQ ID NO:246), a second amino acid sequence being at least 70%, optionally at least 80%, preferably at least 85%, more preferably at least 90% and most preferably at least 95% homologous to a polypeptide having the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) corresponding to amino acids 66-198 of HUMUMOD_P6 (SEQ ID NO:246), a third amino acid sequence being at least 90% homologous LRGWYRFVGQGGARMAETCVPVLRCNTAAPMWLNGTHPSSDEGIVSRKACAHWSGHC CLWDASVQVKACAGGYYVYNLTAPPECHLAYCTDPSSVEGTCEECSIDEDCKSNNGRW HCQCKQDFNIT corresponding to amino acids 66-191 of Q6ZS84_HUMAN (SEQ ID NO:245), which also corresponds to amino acids 199-324 of HUMUMOD_P6 (SEQ ID NO:246), and a fourth amino acid sequence being at least 70%, optionally at least 80%, preferably at least 85%, more preferably at least 90% and most preferably at least 95% homologous to a polypeptide having the sequence APEHKPGPVSSPPISSGLMTSPPISSVLRARHKPQISHRYLPPGAQAGMWGQ (SEQ ID NO:394) corresponding to amino acids 325-376 of HUMUMOD_P6 (SEQ ID NO:246), wherein said first amino acid sequence, second amino acid sequence, third amino acid sequence and fourth amino acid sequence are contiguous and in a sequential order.

B. An isolated polypeptide encoding for an edge portion of HUMUMOD_P6 (SEQ ID NO:246), comprising an amino acid sequence being at least 70%, optionally at least about 80%, preferably at least about 85%, more preferably at least about 90% and most preferably at least about 95% homologous to the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) of HUMUMOD_P6 (SEQ ID NO:246).

C. An isolated polypeptide encoding for an edge portion of HUMUMOD_P6 (SEQ ID NO:246), comprising an amino acid sequence being at least 70%, optionally at least about 80%, preferably at least about 85%, more preferably at least about 90% and most preferably at least about 95% homologous to the sequence APEHKPGPVSSPPISSGLMTSPPISSVLRARHKPQISHRYLPPGAQAGMWGQ (SEQ ID NO:394) of HUMUMOD_P6 (SEQ ID NO:246).

The localization of the variant protein was determined according to results from a number of different software programs and analyses, including analyses from SignalP and other specialized programs. The variant protein is believed to be secreted.

Variant protein HUMUMOD_P6 (SEQ ID NO:246) also has the following non-silent SNPs (Single Nucleotide Polymorphisms) as listed in Table 98, (given according to their position(s) on the amino acid sequence, with the alternative amino acid(s) listed).

TABLE 98
Amino acid mutations

| SNP position(s) on amino acid | Alternative |
|---|---|
| sequence | amino acid(s) |
| 142 | R -> Q |

The glycosylation sites of variant protein HUMUMOD_P6 (SEQ ID NO:246), as compared to the known protein Uromodulin precursor (SEQ ID NO:242), are described in Table 99 (given according to their position(s) on the amino acid sequence in the first column; the second column indicates whether the glycosylation site is present in the variant protein; and the last column indicates whether the position is different on the variant protein).

TABLE 99
Glycosylation site(s)

| Position(s) on known amino | Present in | Position(s) on |
|---|---|---|
| acid sequence | variant protein? | variant protein |
| 38 | Yes | 38 |
| 76 | Yes | 76 |
| 80 | Yes | 80 |
| 232 | Yes | 232 |
| 275 | Yes | 275 |
| 322 | Yes | 322 |
| 396 | No | |

Variant protein HUMUMOD_P6 (SEQ ID NO:246) is encoded by the transcript HUMUMOD_T11 (SEQ ID NO:190), for which the coding portion starts at position 67 and ends at position 1194. The transcript also has the following SNPs as listed in Table 100 (given according to their position on the nucleotide sequence, with the alternative nucleic acid listed; the last column indicates whether the SNP is known or not; the presence of known SNPs in variant protein HUMUMOD_P6 (SEQ ID NO:246) sequence provides support for the deduced sequence of this variant protein according to the present invention).

TABLE 100
Nucleic acid SNPs

| SNP position(s) on nucleotide | Alternative |
|---|---|
| sequence | nucleic acid(s) |
| 491 | G -> A |
| 588 | T -> C |
| 858 | G -> A |
| 951 | G -> A |
| 1466 | C -> |
| 1553 | G -> T |
| 1945 | A -> G |

Variant protein HUMUMOD_P7 (SEQ ID NO:247) according to the present invention is encoded by transcript HUMUMOD_T13 (SEQ ID NO:191). One or more alignments to one or more previously published protein sequences are given in the alignment table on the attached CD-ROM. A brief description of the relationship of the variant protein according to the present invention to each such aligned protein is as follows:

1. Comparison report between HUMUMOD_P7 (SEQ ID NO:247) and UROM_HUMAN (SEQ ID NO:242):

A. An isolated chimeric polypeptide encoding for HUMUMOD_P7 (SEQ ID NO:247), comprising a first amino acid sequence being at least 90% homologous to MGQPSLTWMLMVVVASWFITTAATDTSEARWCSECHSNATCTEDEAVTTCTCQEGFTG DGLTCVDLDECAIPGAHNCSANSSCVNTPGSFSCVCPEGFRLSPGLGCTDVDECAEPGLS HCHALATCVNVVGSYLCVCPAGYRGDGWHCECSPGSCGPGLDCVPEGDALVCADPCQ AHRTLDEYWRSTEYGEGYACDTDLRGWYRFVGQGGARMAETCVPVLRCNTAAPMWL NGTHPSSDEGIVSRKACAHWSGHCCLWDASVQVKACAGGYYVYNLTAPPECHLAYCT DPSSVEGTCEECSIDEDCKSNNGRWHCQCKQDFNITDISLLEHRLECGANDMKVSLGKC QLKSLGFDKVFMYLSDSRCSGFNDRDNRDWVSVVTPARDGPCGTVLTRNETHATYSNT LYLADEIIIRDLNIKINFACSYPLDMKVSLKTALQPMVS corresponding to amino acids 1-444 of UROM_HUMAN (SEQ ID NO:242), which also corresponds to amino acids 1-444 of HUMUMOD_P7 (SEQ ID NO:247), a second amino acid sequence being at least 70%, optionally at least 80%, preferably at least 85%, more preferably at least 90% and most preferably at least 95% homologous to a polypeptide having the sequence QTQERMLGTDEGAQLTWRKKSAHCKLLAGTSPGLAVSWSNVAPSSAC (SEQ ID NO:397) corresponding to amino acids 445-491 of HUMUMOD_P7 (SEQ ID NO:247), and a third amino acid sequence being at least 90% homologous to ALNIRVGGTGMFTVRMALFQTPSYTQPYQGSSVTLSTEAFLYVGTMLDGGDLSRFALLM TNCYATPSSNATDPLKYFIIQDRCPHTRDSTIQVVENGESSQGRFSVQMFRFAGNYDLVY LHCEVYLCDTMNEKCKPTCSGTRFRSGSVIDQSRVLNLGPITRKGVQATVSRAFSSLGLL KVWLPLLLSATLTLTFQ corresponding to amino acids 445-640 of UROM_HUMAN (SEQ ID NO:242), which also corresponds to amino acids 492-687 of HUMUMOD_P7 (SEQ ID NO:247), wherein said first amino acid sequence, second amino acid sequence and third amino acid sequence are contiguous and in a sequential order.

B. An isolated polypeptide encoding for an edge portion of HUMUMOD_P7 (SEQ ID NO:247), comprising an amino acid sequence being at least 70%, optionally at least about 80%, preferably at least about 85%, more preferably at least about 90% and most preferably at least about 95% homologous to the sequence QTQERMLGTDEGAQLTWRKKSAHCKLLAGTSPGLAVSWSNVAPSSAC (SEQ ID NO:397) of HUMUMOD_P7 (SEQ ID NO:247).

4. Comparison report between HUMUMOD_P7 (SEQ ID NO:247) and Q8IYG0_HUMAN (SEQ ID NO:244):

A. An isolated chimeric polypeptide encoding for HUMUMOD_P7 (SEQ ID NO:247), comprising a first amino acid sequence being at least 90% homologous to MGQPSLTWMLMVVVASWFITTAATDTSEARWCSECHSNATCTEDEAVTTCTCQEGFTG DGLTCVDLDECAIPGAHNCSANSSCVNTPGSFSCVCPEGFRLSPGLGCTDVDECAEPGLS HCHALATCVNVVGSYLCVCPAGYRGDGWHCECSPGSCGPGLDCVPEGDALVCADPCQ AHRTLDEYWRSTEYGEGYACDTDLRGWYR corresponding to amino acids 1-204 of Q8IYG0_HUMAN (SEQ ID NO:244), which also corresponds to amino acids 1-204 of HUMUMOD_P7 (SEQ ID NO:247), a second amino acid sequence being at least 70%, optionally at least 80%, preferably at least 85%, more preferably at least 90% and most preferably at least 95% homologous to a polypeptide having the sequence FVGQGGARMAETCVPVLRCNTAAPMWLNGT (SEQ ID NO:395) corresponding to amino acids 205-234 of HUMUMOD_P7 (SEQ ID NO:247), a third amino acid sequence being at least 90% homologous to HPSSDEGIVSRKCAHWSGHCCLWDASVQVKACAGGYYVYNLTAPPECHLAYCTDPSS VEGTCEECSIDEDCKSNNGRWHCQCKQDFNITDISLLEHRLECGANDMKVSLGKCQLKS LGFDKVFMYLSDSRCSGFNDRDNRDWVSVVTPARDGPCGTVLTRNETHATYSNTLYLA DEIIIRDLNIKINFACSYPLDMKVSLKTALQPMVS corresponding to amino acids 206-415 of Q8IYG0_HUMAN (SEQ ID NO:244), which also corresponds to amino acids 235-444 of HUMUMOD_P7 (SEQ ID NO:247), a fourth amino acid sequence being at least 70%, optionally at least 80%, preferably at least 85%, more preferably at least 90% and most preferably at least 95% homologous to a polypeptide having the sequence QTQERMLGTDEGAQLTWRKKSAHCKLLAGTSPGLAVSWSNVAPS SAC (SEQ ID NO:397) corresponding to amino acids 445-491 of HUMUMOD_P7 (SEQ ID NO:247), and a fifth amino acid sequence being at least 90% homologous to ALNIRVGGTGMFTVRMALFQTPSYTQPYQGSSVTLSTEAFLYVGTMLDGGDLSRFALLM TNCYATPSSNATDPLKYFIIQDRCPHTRDSTIQVVENGESSQGRFSVQMFRFAGNYDLVY LHCEVYLCDTMNEKCKPTCSGTRFRSGSVIDQSRVLNLGPITRKGVQATVSRAFSSLGLL KVWLPLLLSATLTLTFQ corresponding to amino acids 416-611 of Q8IYG0_HUMAN (SEQ ID NO:244), which also corresponds to amino acids 492-687 of HUMUMOD_P7 (SEQ ID NO:247), wherein said first amino acid sequence, second amino acid sequence, third amino acid sequence, fourth amino acid sequence and fifth amino acid sequence are contiguous and in a sequential order.

B. An isolated polypeptide encoding for an edge portion of HUMUMOD_P7 (SEQ ID NO:247), comprising an amino acid sequence being at least 70%, optionally at least about 80%, preferably at least about 85%, more preferably at least about 90% and most preferably at least about 95% homologous to the sequence FVGQGGARMAETCVPVLRCNTAAPMWLNGT (SEQ ID NO:395) of HUMUMOD_P7 (SEQ ID NO:247).

C. An isolated polypeptide encoding for an edge portion of HUMUMOD_P7 (SEQ ID NO:247), comprising an amino acid sequence being at least 70%, optionally at least about 80%, preferably at least about 85%, more preferably at least about 90% and most preferably at least about 95% homologous to the sequence QTQERMLGTDEGAQLTWRKKSAHCKLLAGTSPGLAVSWSNVAPS SAC (SEQ ID NO:397) of HUMUMOD_P7 (SEQ ID NO:247).

5. Comparison report between HUMUMOD_P7 (SEQ ID NO:247) and Q6ZS84_HUMAN (SEQ ID NO:245):

A. An isolated chimeric polypeptide encoding for HUMUMOD_P7 (SEQ ID NO:247), comprising a first amino acid sequence being at least 90% homologous to MGQPSLTWMLMVVVASWFITTAATDTSEARWCSECHSNATCTEDEAVTTCTCQEGFTG DGLTCVD corresponding to amino acids 1-65 of Q6ZS84_HUMAN (SEQ ID NO:245), which also corresponds to amino acids 1-65 of HUMUMOD_P7 (SEQ ID NO:247), a second amino acid sequence being at least 70%, optionally at least 80%, preferably at least 85%, more preferably at least 90% and most preferably at least 95% homologous to a polypeptide having the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) corresponding to amino acids 66-198 of HUMUMOD_P7 (SEQ ID NO:247), a third amino acid sequence being at least 90% homologous LRGWYRFVGQGGARMAETCVPVLRCNTAAPMWLNGTHPSSDEGIVSRKACAHWSGHC CLWDASVQVKACAGGYYVYNLTAPPECHLAYCTDPSSVEGTCEECSIDEDCKSNNGRW HCQCKQDFNITDISLLEHRLECGANDMKVSLGKCQLKSLGFDKVFMYLSDSRCSGFNDR DNRDWVSWTPARDGPCGTVLTRNETHATYSNTLYLADEIIIRDLNIKINFACSYPLDMK VSLKTALQPMVS corresponding to amino acids 66-311 of Q6ZS84_HUMAN (SEQ ID NO:245), which also corresponds to amino acids 199-444 of HUMUMOD_P7 (SEQ ID NO:247), a fourth amino acid sequence being at least 70%, optionally at least 80%, preferably at least 85%, more preferably at least 90% and most preferably at least 95% homologous to a polypeptide having the sequence QTQERMLGTDEGAQLTWRKKSAHCKLLAGTSPGLAVSWSNVAPSSAC (SEQ ID NO:397) corresponding to amino acids 445-491 of HUMUMOD_P7 (SEQ ID NO:247), and a fifth amino acid sequence being at least 90% homologous to ALNIRVGGTGMFTVRMALFQTPSYTQPYQGSSVTLSTEAFLYVGTMLDGGDLSRFALLM TNCYATPSSNATDPLKYFIIQDRCPHTRDSTIQVVENGESSQGRFSVQMFRFAGNYDLVY LHCEVYLCDTMNEKCKPTCSGTRFRSGSVIDQSRVLNLGPITRKGVQATVSRAFSSLGLL KVWLPLLLSATLTLTFQ corresponding to amino acids 312-507 of Q6ZS84_HUMAN (SEQ ID NO:245), which also corresponds to amino acids 492-687 of HUMUMOD_P7 (SEQ ID NO:247), wherein said first amino acid sequence, second amino acid sequence, third amino acid sequence, fourth amino acid sequence and fifth amino acid sequence are contiguous and in a sequential order.

B. An isolated polypeptide encoding for an edge portion of HUMOD_P7 (SEQ ID NO:247), comprising an amino acid sequence being at least 70%, optionally at least about 80%, preferably at least about 85%, more preferably at least about 90% and most preferably at least about 95% homologous to the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) of HUMUMOD_P7 (SEQ ID NO:247).

C. An isolated polypeptide encoding for an edge portion of HUMUMOD_P7 (SEQ ID NO:247), comprising an amino acid sequence being at least 70%, optionally at least about 80%, preferably at least about 85%, more preferably at least about 90% and most preferably at least about 95% homologous to the sequence QTQERMLGTDEGAQLTWRKKSAHCKLLAGTSPGLAVSWSNVAPSSAC (SEQ ID NO:397) of HUMUMOD_P7 (SEQ ID NO:247).

The localization of the variant protein was determined according to results from a number of different software programs and analyses, including analyses from SignalP and other specialized programs. The variant protein is believed to be secreted.

Variant protein HUMUMOD_P7 (SEQ ID NO:247) also has the following non-silent SNPs (Single Nucleotide Polymorphisms) as listed in Table 101, (given according to their position(s) on the amino acid sequence, with the alternative amino acid(s) listed).

TABLE 101
Amino acid mutations

| SNP position(s) on amino acid | Alternative |
|---|---|
| sequence | amino acid(s) |
| 142 | R -> Q |
| 429 | L -> |
| 505 | V -> L |

The glycosylation sites of variant protein HUMUMOD_P7 (SEQ ID NO:247), as compared to the known protein Uromodulin precursor (SEQ ID NO:242), are described in Table 102 (given according to their position(s) on the amino acid sequence in the first column; the second column indicates whether the glycosylation site is present in the variant protein; and the last column indicates whether the position is different on the variant protein).

TABLE 102
Glycosylation site(s)

| Position(s) on known amino | Present in | Position(s) on |
|---|---|---|
| acid sequence | variant protein? | variant protein |
| 38 | Yes | 38 |
| 76 | Yes | 76 |
| 80 | Yes | 80 |
| 232 | Yes | 232 |
| 275 | Yes | 275 |
| 322 | Yes | 322 |
| 396 | Yes | 396 |

Variant protein HUMUMOD_P7 (SEQ ID NO:247) is encoded by the transcript HUMUMOD_T13 (SEQ ID NO:191), for which the coding portion starts at position 67 and ends at position 2127. The transcript also has the following SNPs as listed in Table 103 (given according to their position on the nucleotide sequence, with the alternative nucleic acid listed; the last column indicates whether the SNP is known or not; the presence of known SNPs in variant protein HUMUMOD_P7 (SEQ ID NO:247) sequence provides support for the deduced sequence of this variant protein according to the present invention).

TABLE 103
Nucleic acid SNPs

| SNP position(s) on nucleotide | |
|---|---|
| sequence | Alternative nucleic acid(s) |
| 491 | G -> A |
| 588 | T -> C |
| 858 | G -> A |
| 951 | G -> A |
| 1351 | C -> |
| 1579 | G -> T |
| 1971 | A -> G |

Variant protein HUMUMOD_P14 (SEQ ID NO:248) according to the present invention is encoded by transcript HUMUMOD_T20 (SEQ ID NO:192) One or more alignments to one or more previously published protein sequences are given in the alignment table on the attached CD-ROM. A brief description of the relationship of the variant protein according to the present invention to each such aligned protein is as follows:

1. Comparison report between HUMUMOD_P14 (SEQ ID NO:248) and UROM_HUMAN (SEQ ID NO:242):

A. An isolated chimeric polypeptide encoding for HUMUMOD_P14 (SEQ ID NO:248), comprising a first amino acid sequence being at least 90% homologous to MGQPSLTWMLMVVVASWFITTAATDTSEARWCSECHSNATCTEDEAVTTCTCQEGFTG DGLTCVDLDECAIPGAHNCSANSSCVNTPGSFSCVCPEGFRLSPGLGCTDVDECAEPGLS HCHALATCVNVVGSYLCVCPAGYRGDGWHCECSPGSCGPGLDCVPEGDALVCADPCQ AHRTLDEYWRSTEYGEGYACDTDLRGWYRFVGQGGARMAETCVPVLRCNTAAPMWL NGTHPSSDEGIVSRKACAHWSGHCCLWDASVQVKACAGGYYVYNLTAPPECHLAYCT DPSSVEGTCEECSIDEDCKSNNGRWHCQCKQDFNITDISLLEHRLECGANDMKVSLGKC QLKSLGFDKVFMYLSDSRCSGFNDRDNRDWVSVVTPARDGPCGTVLTRNETHATYSNT LYLADEIIIRDLNIKINFACSYPLDMKVSLKTALQPMV corresponding to amino acids 1-443 of UROM_HUMAN (SEQ ID NO:242), which also corresponds to amino acids 1-443 of HUMUMOD_P14 (SEQ ID NO:248), and a second amino acid sequence being at least 90% homologous to RCPHTRDSTIQVVENGESSQGRFSVQMFRFAGNYDLVYLHCEVYLCDTMNEKCKPTCSG TRFRSGSVIDQSRVLNLGPITRKGVQATVSRAFSSLGLLKVWLPLLLSATLTLTFQ corresponding to amino acids 526-640 of UROM_HUMAN (SEQ ID NO:242), which also corresponds to amino acids 444-558 of HUMUMOD_P14 (SEQ ID NO:248), wherein said first amino acid sequence and second amino acid sequence are contiguous and in a sequential order.

B. An isolated chimeric polypeptide encoding for an edge portion of HUMUMOD_P14 (SEQ ID NO:248), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VR, having a structure as follows: a sequence starting from any of amino acid numbers 443-x to 443; and ending at any of amino acid numbers 444+((n−2)−x), in which x varies from 0 to n−2.

4. Comparison report between HUMUMOD_P14 (SEQ ID NO:248) and Q8IYG0_HUMAN (SEQ ID NO:244):

A. An isolated chimeric polypeptide encoding for HUMUMOD_P14 (SEQ ID NO:248), comprising a first amino acid sequence being at least 90% homologous to MGQPSLTWMLMVVVASWFITTAATDTSEARWCSECHSNATCTEDEAVTTCTCQEGFTG DGLTCVDLDECAIPGAHNCSANSSCVNTPGSFSCVCPEGFRLSPGLGCTDVDECAEPGLS HCHALATCVNVVGSYLCVCPAGYRGDGWHCECSPGSCGPGLDCVPEGDALVCADPCQ AHRTLDEYWRSTEYGEGYACDTDLRGWYR corresponding to amino acids 1-204 of Q8IYG0_HUMAN (SEQ ID NO:244), which also corresponds to amino acids 1 204 of HUMUMOD_P14 (SEQ ID NO:248), a second amino acid sequence being at least 70%, optionally at least 80%, preferably at least 85%, more preferably at least 90% and most preferably at least 95% homologous to a polypeptide having the sequence FVGQGGARMAETCVPVLRCNTAAPMWLNGT (SEQ ID NO:395) corresponding to amino acids 205-234 of HUMUMOD_P14 (SEQ ID NO:248), a third amino acid sequence being at least 90% homologous to HPSSDEGIVSRKACAHWSGHCCLWDASVQVKACAGGYYVYNLTAPPECHLAYCTDPSS VEGTCEECSIDEDCKSNNGRWHCQCKQDFNITDISLLEHRLECGANDMKVSLGKCQLKS LGFDKVFMYLSDSRCSGFNDRDNRDWVSVVTPARDGPCGTVLTRNETHATYSNTLYLA DEIIIRDLNIKINFACSYPLDMKVSLKTALQPMV corresponding to amino acids 206-414 of Q8IYG0_HUMAN (SEQ ID NO:244), which also corresponds to amino acids 235-443 of HUMUMOD_P14 (SEQ ID NO:248), and a fourth amino acid sequence being at least 90% homologous to RCPHTRDSTIQVVENGESSQGRFSVQMFRFAGNYDLVYLHCEVYLCDTMNEKCKPTCSG TRFRSGSVIDQSRVLNLGPITRKGVQATVSRAFSSLGLLKVWLPLLLSATLTLTFQ corresponding to amino acids 497-611 of Q8IYG0_HUMAN (SEQ ID NO:244), which also corresponds to amino acids 444-558 of HUMUMOD_P14 (SEQ ID NO:248), wherein said first amino acid sequence, second amino acid sequence, third amino acid sequence and fourth amino acid sequence are contiguous and in a sequential order.

B. An isolated polypeptide encoding for an edge portion of HUMUMOD_P14 (SEQ ID NO:248), comprising an amino acid sequence being at least 70%, optionally at least about 80%, preferably at least about 85%, more preferably at least about 90% and most preferably at least about 95% homologous to the sequence FVGQGGARMAETCVPVLRCNTAAPMWLNGT (SEQ ID NO:395) of HUMUMOD_P14 (SEQ ID NO:248).

C. An isolated chimeric polypeptide encoding for an edge portion of HUMUMOD_P14 (SEQ ID NO:248), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VR, having a structure as follows: a sequence starting from any of amino acid numbers 443-x to 443; and ending at any of amino acid numbers 444+((n−2)−x), in which x varies from 0 to n−2.

5. Comparison report between HUMUMOD_P14 (SEQ ID NO:248) and Q6ZS84_HUMAN (SEQ ID NO:245):

A. An isolated chimeric polypeptide encoding for HUMUMOD_P14 (SEQ ID NO:248), comprising a first amino acid sequence being at least 90% homologous to MGQPSLTWMLMVVVASWFITTAATDTSEARWCSECHSNATCTEDEAVTTCTCQEGFTG DGLTCVD corresponding to amino acids 1-65 of Q6ZS84_HUMAN (SEQ ID NO:245), which also corresponds to amino acids 1-65 of HUMUMOD_P14 (SEQ ID NO:248), a second amino acid sequence being at least 70%, optionally at least 80%, preferably at least 85%, more preferably at least 90% and most preferably at least 95% homologous to a polypeptide having the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) corresponding to amino acids 66-198 of HUMUMOD_P14 (SEQ ID NO:248), a third amino acid sequence being at least 90% homologous to LRGWYRFVGQGGARMAETCVPVLRCNTAAPMWLNGTHPSSDEGWSRKACAHWSGHC CLWDASVQVKACAGGYYVYNLTAPPECHLAYCTDPSSVEGTCEECSIDEDCKSNNGRW HCQCKQDFNITDISLLEHRLECGANDMKVSLGKCQLKSLGFDKVFMYLSDSRCSGFNDR DNRDWVSVVTPARDGPCGTVLTRNETHATYSNTLYLADEIIIRDLNIKNFACSYPLDMK VSLKTALQPMV corresponding to amino acids 66-310 of Q6ZS84_HUMAN (SEQ ID NO:245), which also corresponds to amino acids 199-443 of HUMUMOD_P14 (SEQ ID NO:248), and a fourth amino acid sequence being at least 90% homologous to RCPHTRDSTIQVVENGESSQGRFSVQMFRFAGNYDLVYLHCEVYLCDTMNEKCKPTCSG TRFRSGSVIDQSRVLNLGPITRKGVQATVSRAFSSLGLLKVWLPLLLSATLTLTFQ corresponding to amino acids 393-507 of Q6ZS84_HUMAN (SEQ ID NO:245), which also corresponds to amino acids 444-558 of HUMUMOD_P14 (SEQ ID NO:248), wherein said first amino acid sequence, second amino acid sequence, third amino acid sequence and fourth amino acid sequence are contiguous and in a sequential order.

B. An isolated polypeptide encoding for an edge portion of HUMUMOD_P14 (SEQ ID NO:248), comprising an amino acid sequence being at least 70%, optionally at least about 80%, preferably at least about 85%, more preferably at least about 90% and most preferably at least about 95% homologous to the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) of HUMUMOD_P14 (SEQ ID NO:248).

C. An isolated chimeric polypeptide encoding for an edge portion of HUMUMOD_P14 (SEQ ID NO:248), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise VR, having a structure as follows: a sequence starting from any of amino acid numbers 443-x to 443; and ending at any of amino acid numbers 444+((n−2)−x), in which x varies from 0 to n−2.

The localization of the variant protein was determined according to results from a number of different software programs and analyses, including analyses from SignalP and other specialized programs. The variant protein is believed to be secreted.

Variant protein HUMUMOD_P14 (SEQ ID NO:248) also has the following non-silent SNPs (Single Nucleotide Polymorphisms) as listed in Table 104, (given according to their position(s) on the amino acid sequence, with the alternative amino acid(s) listed).

TABLE 104
Amino acid mutations

| SNP position(s) on amino acid | |
|---|---|
| sequence | Alternative amino acid(s) |
| 142 | R -> Q |
| 429 | L -> |

The glycosylation sites of variant protein HUMUMOD_P14 (SEQ ID NO:248), as compared to the known protein Uromodulin precursor (SEQ ID NO:242), are described in Table 105 (given according to their position(s) on the amino acid sequence in the first column; the second column indicates whether the glycosylation site is present in the variant protein; and the last column indicates whether the position is different on the variant protein).

TABLE 105
Glycosylation site(s)

| Position(s) on known amino | Present in | Position(s) on |
|---|---|---|
| acid sequence | variant protein? | variant protein |
| 38 | Yes | 38 |
| 76 | Yes | 76 |
| 80 | Yes | 80 |
| 232 | Yes | 232 |
| 275 | Yes | 275 |
| 322 | Yes | 322 |
| 396 | Yes | 396 |

Variant protein HUMUMOD_P14 (SEQ ID NO:248) is encoded by the following transcript(s): HUMUMOD_T20 (SEQ ID NO:192), for which the sequence(s) is/are given at the end of the application. The coding portion of transcript HUMUMOD_T20 (SEQ ID NO:192) is shown in bold; this coding portion starts at position 67 and ends at position 1740. The transcript also has the following SNPs as listed in Table 106 (given according to their position on the nucleotide sequence, with the alternative nucleic acid listed).

TABLE 106
Nucleic acid SNPs

| SNP position(s) on nucleotide | |
|---|---|
| sequence | Alternative nucleic acid(s) |
| 491 | G -> A |
| 588 | T -> C |
| 858 | G -> A |
| 951 | G -> A |
| 1351 | C -> |
| 1584 | A -> G |

Variant protein HUMUMOD_P24 (SEQ ID NO:249) according to the present invention is encoded by transcript HUMUMOD_T31 (SEQ ID NO:193) One or more alignments to one or more previously published protein sequences are given in the alignment table on the attached CD-ROM. A brief description of the relationship of the variant protein according to the present invention to each such aligned protein is as follows:

1. Comparison report between HUMUMOD_P24 (SEQ ID NO:249) and UROM_HUMAN (SEQ ID NO:242):

A. An isolated chimeric polypeptide encoding for HUMUMOD_P24 (SEQ ID NO:249), comprising a first amino acid sequence being at least 90% homologous to MGQPSLTWMLMVVVASWFITTAATDTSEARWCSECHSNATCTEDEAVTTCTCQEGFTG DGLTCVDLDECAIPGAHNCSANSSCVNTPGSFSCVCPEGFRLSPGLGCTDVDECAEPGLS HCHALATCVNVVGSYLCVCPAGYRGDGWHCECSPGSCGPGLDCVPEGDALVCADPCQ AHRTLDEYWRSTEYGEGYACDTDLRGWYRFVGQGGARMAETCVPVLRCNTAAPMWL NGTHPSSDEGIVSRKACAHWSGHCCLWDASVQVKACAGGYYVYNLTAPPECHLAYCT DPSSVEGTCEECSIDEDCKSNNGRWHCQCKQDFNITDISLLEHRLECGANDMKVSLGKC QLKSLGFDKVFMYLSDSRCSGFNDRDNRDWVSVVTPARDGPCGTVLTRNETHATYSNT LYLADEIIIRDLNIKINFACSYPLDMKVSLKTALQPMV corresponding to amino acids 1-443 of UROM_HUMAN (SEQ ID NO:242), which also corresponds to amino acids 1-443 of HUMUMOD_P24 (SEQ ID NO:249), and a second amino acid sequence being at least 70%, optionally at least 80%, preferably at least 85%, more preferably at least 90% and most preferably at least 95% homologous to a polypeptide having the sequence RCGQRGSLGPLDGSNPKPLNHELPCQLPPTGSWE (SEQ ID NO:402) corresponding to amino acids 444-477 of HUMOD_P24 (SEQ ID NO:249), wherein said first amino acid sequence and second amino acid sequence are contiguous and in a sequential order.

B. An isolated polypeptide encoding for an edge portion of HUMUMOD_P24 (SEQ ID NO:249), comprising an amino acid sequence being at least 70%, optionally at least about 80%, preferably at least about 85%, more preferably at least about 90% and most preferably at least about 95% homologous to the sequence RCGQRGSLGPLDGSNPKPLNHELPCQLPPTGSWE (SEQ ID NO:402) of HUMUMOD_P24 (SEQ ID NO:249).

4. Comparison report between HUMUMOD_P24 (SEQ ID NO:249) and Q8IYG0_HUMAN (SEQ ID NO:244):

A. An isolated chimeric polypeptide encoding for HUMUMOD_P24 (SEQ ID NO:249), comprising a first amino acid sequence being at least 90% homologous to MGQPSLTWMLMVVVASWFITTAATDTSEARWCSECHSNATCTEDEAVTTCTCQEGFTG DGLTCVDLDECAIPGAHNCSANSSCVNTPGSFSCVCPEGFRLSPGLGCTDVDECAEPGLS HCHALATCVNVVGSYLCVCPAGYRGDGWHCECSPGSCGPGLDCVPEGDALVCADPCQ AHRTLDEYWRSTEYGEGYACDTDLRGWYR corresponding to amino acids 1-204 of Q8IYG0_HUMAN (SEQ ID NO:244), which also corresponds to amino acids 1-204 of HUMUMOD_P24 (SEQ ID NO:249), a second amino acid sequence being at least 70%, optionally at least 80%, preferably at least 85%, more preferably at least 90% and most preferably at least 95% homologous to a polypeptide having the sequence FVGQGGARMAETCVPVLRCNTAAPMWLNGT (SEQ ID NO:395) corresponding to amino acids 205-234 of HUMUMOD_P24 (SEQ ID NO:249), a third amino acid sequence being at least 90% homologous to HPSSDEGIVSRKACAHWSGHCCLWDASVQVKACAGGYYVYNLTAPPECHLAYCTDPSS VEGTCEECSIDEDCKSNNGRWHCQCKQDFNITDISLLEHRLECGANDMKVSLGKCQLKS LGFDKVFMYLSDSRCSGFNDRDNRDWVSVVTPARDGPCGTVLTRNETHATYSNTLYLA DEIIIRDLNIKINFACSYPLDMKVSLKTALQPMV corresponding to amino acids 206-414 of Q8IYG0_HUMAN (SEQ ID NO:244), which also corresponds to amino acids 235-443 of HUMUMOD_P24 (SEQ ID NO:249), and a fourth amino acid sequence being at least 70%, optionally at least 80%, preferably at least 85%, more preferably at least 90% and most preferably at least 95% homologous to a polypeptide having the sequence RCGQRGSLGPLDGSNPKPLNHELPCQLPPTGSWE (SEQ ID NO:402) corresponding to amino acids 444-477 of HUMUMOD_P24 (SEQ ID NO:249), wherein said first amino acid sequence, second amino acid sequence, third amino acid sequence and fourth amino acid sequence are contiguous and in a sequential order.

B. An isolated polypeptide encoding for an edge portion of HUMMOD_P24 (SEQ ID NO:249), comprising an amino acid sequence being at least 70%, optionally at least about 80%, preferably at least about 85%, more preferably at least about 90% and most preferably at least about 95% homologous to the sequence FVGQGGARMAETCVPVLRCNTAAPMWLNGT (SEQ ID NO:395) of HUMUMOD_P24 (SEQ ID NO:249).

C. An isolated polypeptide encoding for an edge portion of HUMUMOD_P24 (SEQ ID NO:249), comprising an amino acid sequence being at least 70%, optionally at least about 80%, preferably at least about 85%, more preferably at least about 90% and most preferably at least about 95% homologous to the sequence RCGQRGSLGPLDGSNPKPLNHELPCQLPPTGSWE (SEQ ID NO:402) of HUMUMOD_P24 (SEQ ID NO:249).

5. Comparison report between HUMUMOD_P24 (SEQ ID NO:249) and Q6ZS84_HUMAN (SEQ ID NO:245):

A. An isolated chimeric polypeptide encoding for HUMUMOD_P24 (SEQ ID NO:249), comprising a first amino acid sequence being at least 90% homologous to MGQPSLTWMLMVVVASWFITTAATDTSEARWCSECHSNATCTEDEAVTTCTCQEGFTG DGLTCVD corresponding to amino acids 1-65 of Q6ZS84_HUMAN (SEQ ID NO:245), which also corresponds to amino acids 1-65 of HUMUMOD_P24 (SEQ ID NO:249), a second amino acid sequence being at least 70%, optionally at least 80%, preferably at least 85%, more preferably at least 90% and most preferably at least 95% homologous to a polypeptide having the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) corresponding to amino acids 66-198 of HUMUMOD_P24 (SEQ ID NO:249), a third amino acid sequence being at least 90% homologous to LRGWYRFVGQGGARMAETCVPVLRCNTAAPMWLNGTHPSSDEGIVSRKACAHWSGHC CLWDASVQVKACAGGYYVYNLTAPPECHLAYCTDPSSVEGTCEECSIDEDCKSNNGRW HCQCKQDFNITDISLLEHRLECGANDMKVSLGKCQLKSLGFDKVFMYLSDSRCSGFNDR DNRDWVSVVTPARDGPCGTVLTRNETHATYSNTLYLADEIIIDLNIKINFACSYPLDMK VSLKTALQPMV corresponding to amino acids 66-310 of Q6ZS84_HUMAN (SEQ ID NO:245), which also corresponds to amino acids 199-443 of HUMUMOD_P24 (SEQ ID NO:249), and a fourth amino acid sequence being at least 70%, optionally at least 80%, preferably at least 85%, more preferably at least 90% and most preferably at least 95% homologous to a polypeptide having the sequence RCGQRGSLGPLDGSNPKPLNHELPCQLPPTGSWE (SEQ ID NO:402) corresponding to amino acids 444-477 of HUMOD_P24 (SEQ ID NO:249), wherein said first amino acid sequence, second amino acid sequence, third amino acid sequence and fourth amino acid sequence are contiguous and in a sequential order.

B. An isolated polypeptide encoding for an edge portion of HUMUMOD_P24 (SEQ ID NO:249), comprising an amino acid sequence being at least 70%, optionally at least about 80%, preferably at least about 85%, more preferably at least about 90% and most preferably at least about 95% homologous to the sequence LDECAIPGAHNCSANSSCVNTPGSFSCVCPEGFRLSPGLGCTDVDECAEPGLSHCHALAT CVNVVGSYLCVCPAGYRGDGWHCECSPGSCGPGLDCVPEGDALVCADPCQAHRTLDEY WRSTEYGEGYACDTD (SEQ ID NO:396) of HUMUMOD_P24 (SEQ ID NO:249).

C. An isolated polypeptide encoding for an edge portion of HUMUMOD_P24 (SEQ ID NO:249), comprising an amino acid sequence being at least 70%, optionally at least about 80%, preferably at least about 85%, more preferably at least about 90% and most preferably at least about 95% homologous to the sequence RCGQRGSLGPLDGSNPKPLNHELPCQLPPTGSWE (SEQ ID NO:402) of HUMUMOD_P24 (SEQ ID NO:249).

The localization of the variant protein was determined according to results from a number of different software programs and analyses, including analyses from SignalP and other specialized programs. The variant protein is believed to be secreted.

Variant protein HUMUMOD_P24 (SEQ ID NO:249) also has the following non-silent SNPs (Single Nucleotide Polymorphisms) as listed in Table 107, (given according to their position(s) on the amino acid sequence, with the alternative amino acid(s) listed; the last column indicates whether the SNP is known or not; the presence of known SNPs in variant protein HUMUMOD_P24 (SEQ ID NO:249) sequence provides support for the deduced sequence of this variant protein according to the present invention).

TABLE 107
Amino acid mutations

| SNP position(s) on amino acid | |
|---|---|
| sequence | Alternative amino acid(s) |
| 142 | R -> Q |
| 429 | L -> |

The glycosylation sites of variant protein HUMUMOD_P24 (SEQ ID NO:249), as compared to the known protein Uromodulin precursor (SEQ ID NO:242), are described in Table 108 (given according to their position(s) on the amino acid sequence in the first column; the second column indicates whether the glycosylation site is present in the variant protein; and the last column indicates whether the position is different on the variant protein).

TABLE 108
Glycosylation site(s)

| Position(s) on known amino | Present in | Position(s) on |
|---|---|---|
| acid sequence | variant protein? | variant protein |
| 38 | Yes | 38 |
| 76 | Yes | 76 |
| 80 | Yes | 80 |
| 232 | Yes | 232 |
| 275 | Yes | 275 |
| 322 | Yes | 322 |
| 396 | Yes | 396 |

Variant protein HUMUMOD_P24 (SEQ ID NO:249) is encoded by the following transcript(s): HUMUMOD_T31 (SEQ ID NO:193), for which the sequence(s) is/are given at the end of the application. The coding portion of transcript HUMUMOD_T31 (SEQ ID NO:193) is shown in bold; this coding portion starts at position 67 and ends at position 1497. The transcript also has the following SNPs as listed in Table 109 (given according to their position on the nucleotide sequence, with the alternative nucleic acid listed).

TABLE 109
Nucleic acid SNPs

| SNP position(s) on nucleotide | |
|---|---|
| sequence | Alternative nucleic acid(s) |
| 491 | G -> A |
| 588 | T -> C |
| 858 | G -> A |
| 951 | G -> A |
| 1351 | C -> |
| 1804 | C -> A |

As noted above, cluster HUMUMOD features 49 segments, which were listed in Table 93. These segments are portions of nucleic acid sequences which are described herein separately because they are of particular interest. A description of some of the segments according to the present invention is now provided.

Segment cluster HUMUMOD_N55 (SEQ ID NO:196) according to the present invention is supported by 51 libraries. The number of libraries was determined as previously described. This segment can be found in the following transcript(s): HUMUMOD_T11 (SEQ ID NO:190), HUMUMOD_T13 (SEQ ID NO:191), HUMUMOD_T20 (SEQ ID NO:1192) and HUMUMOD_T31 (SEQ ID NO:193). Table 110 below describes the starting and ending position of this segment on each transcript.

TABLE 110
Segment location on transcripts

| | | Segment |
|---|---|---|
| | Segment | ending |
| Transcript name | starting position | position |
| HUMUMOD_T11 (SEQ ID NO: 190) | 1364 | 1512 |
| HUMUMOD_T13 (SEQ ID NO: 191) | 1249 | 1397 |
| HUMUMOD_T20 (SEQ ID NO: 192) | 1249 | 1397 |
| HUMUMOD_T31 (SEQ ID NO: 193) | 1249 | 1397 |

Segment cluster HUMUMOD_N56 (SEQ ID NO:197) according to the present invention is supported by 8 libraries. The number of libraries was determined as previously described. This segment can be found in the following transcript(s): HUMUMOD_T31 (SEQ ID NO:193). Table 111 below describes the starting and ending position of this segment on each transcript.

TABLE 111
Segment location on transcripts

| | Segment | Segment |
|---|---|---|
| Transcript name | starting position | ending position |
| HUMUMOD_T31 | 1398 | 2369 |
| (SEQ ID NO: 193) | | |

Segment cluster HUMUMOD_N58 (SEQ ID NO:198) according to the present invention is supported by 5 libraries. The number of libraries was determined as previously described. This segment can be found in the following transcript(s): HUMUMOD_T13 (SEQ ID NO:191). Table 112 below describes the starting and ending position of this segment on each transcript.

TABLE 112
Segment location on transcripts

| | Segment | Segment |
|---|---|---|
| Transcript name | starting position | ending position |
| HUMUMOD_T13 | 1398 | 1538 |
| (SEQ ID NO: 191) | | |

Segment cluster HUMUMOD_N61 (SEQ ID NO:199) according to the present invention is supported by 61 libraries. The number of libraries was determined as previously described. This segment can be found in the following transcript(s): HUMUMOD_T11 (SEQ ID NO:190) and HUMUMOD_T13 (SEQ ID NO:191). Table 113 below describes the starting and ending position of this segment on each transcript.

TABLE 113
Segment location on transcripts

| | | Segment |
|---|---|---|
| | Segment | ending |
| Transcript name | starting position | position |
| HUMUMOD_T11 (SEQ ID NO: 190) | 1513 | 1758 |
| HUMUMOD_T13 (SEQ ID NO: 191) | 1539 | 1784 |

Segment cluster HUMUMOD_N64 (SEQ ID NO:200) according to the present invention is supported by 53 libraries. The number of libraries was determined as previously described. This segment can be found in the following transcript(s): HUMUMOD_T11 (SEQ ID NO:190), HUMUMOD_T13 (SEQ ID NO:191) and HUMUMOD_T20 (SEQ ID NO:192). Table 114 below describes the starting and ending position of this segment on each transcript.

TABLE 114
Segment location on transcripts

| | | Segment |
|---|---|---|
| | Segment | ending |
| Transcript name | starting position | position |
| HUMUMOD_T11 (SEQ ID NO: 190) | 1759 | 1919 |
| HUMUMOD_T13 (SEQ ID NO: 191) | 1785 | 1945 |
| HUMUMOD_T20 (SEQ ID NO: 192) | 1398 | 1558 |

According to an optional embodiment of the present invention, short segments related to the above cluster are also provided. These segments are up to about 120 bp in length, and so are included in a separate description.

Segment cluster HUMUMOD_N47 (SEQ ID NO:233) according to the present invention is supported by 3 libraries. The number of libraries was determined as previously described. This segment can be found in the following transcript(s): HUMUMOD_T11 (SEQ ID NO:190). Table 115 below describes the starting and ending position of this segment on each transcript.

TABLE 115
Segment location on transcripts

| | Segment | Segment |
|---|---|---|
| Transcript name | starting position | ending position |
| HUMUMOD_T11 | 1040 | 1062 |
| (SEQ ID NO: 190) | | |

Segment cluster HUMUMOD_N48 (SEQ ID NO:234) according to the present invention is supported by 2 libraries. The number of libraries was determined as previously described. This segment can be found in the following transcript(s): HUMUMOD_T11 (SEQ ID NO:190). Table 116 below describes the starting and ending position of this segment on each transcript.

TABLE 116
Segment location on transcripts

| | Segment | Segment |
|---|---|---|
| Transcript name | starting position | ending position |
| HUMUMOD_T11 | 1063 | 1154 |
| (SEQ ID NO: 190) | | |

Expression of Homo sapiens uromodulin (uromucoid, Tamm-Horsfall glycoprotein) (UMOD) HUMUMOD transcripts which are detectable by amplicon as depicted in sequence name HUMUMOD_seg47-48 (SEQ ID NO:252) specifically in kidney tissue

Expression of Homo sapiens uromodulin (uromucoid, Tamm-Horsfall glycoprotein) (UMOD) transcripts detectable by or according to seg47-48—HUMUMOD_seg47-48 (SEQ ID NO:252) amplicon and primers HUMUMOD_seg47-48F (SEQ ID NO:250) and HUMUMOD_seg47-48R (SEQ ID NO:251) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

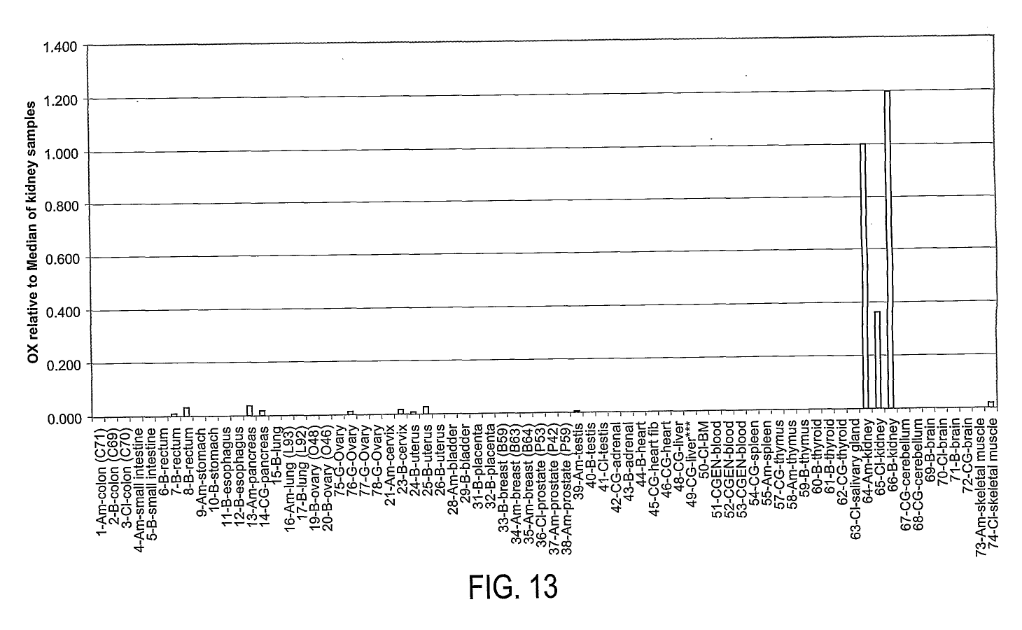

FIG. 13 is a histogram showing relative expression of the above-indicated Homo sapiens uromodulin (uromucoid, Tamm-Horsfall glycoprotein) (UMOD) transcripts in kidney tissue samples as opposed to other tissues.

As is evident from FIG. 13, the expression of Homo sapiens Homo sapiens uromodulin (uromucoid, Tamm-Horsfall glycoprotein) (UMOD) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: HUMUMOD_seg47-48F (SEQ ID NO:250) forward primer; and HUMUMOD_seg47-48R (SEQ ID NO:251) reverse primer.

The present invention also preferably encompasses any amplicon obtained through the use of any suitable primer pair; for example, for the above experiment, the following amplicon was obtained as a non-limiting illustrative example only of a suitable amplicon: HUMUMOD_seg47-48 (SEQ ID NO:252).

Forward Primer (HUMUMOD_seg47-48F
(SEQ ID NO: 250):
GGCCCGGTGAGCAGTC
Reverse Primer (HUMUMOD_seg47-48R
(SEQ ID NO: 251):
CTATGAGAAATCTGAGGCTTGTGC
Amplicon (HUMUMOD_seg47-48 (SEQ ID NO: 252):
GGCCCGGTGAGCAGTCCACCAATCAGCTCAGGCCTGATGACCAGTCCACC
AATCAGCTCAGTACTCCGGGCCAGGCACAAGCCTCAGATTTCTCATAG

Expression of Homo sapiens uromodulin (uromucoid, Tamm-Horsfall glycoprotein) (UMOD) HUMUMOD transcripts which are detectable by amplicon as depicted in sequence name HUMUMOD_seg56 (SEQ ID NO:255) specifically in kidney tissue

Expression of Homo sapiens uromodulin (uromucoid, Tamm-Horsfall glycoprotein) (UMOD) transcripts detectable by or according to seg56—HUMUMOD_seg56 (SEQ ID NO:255) amplicon and primers HUMUMOD_seg56F (SEQ ID NO:253) and HUMUMOD_seg56R (SEQ ID NO:254) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

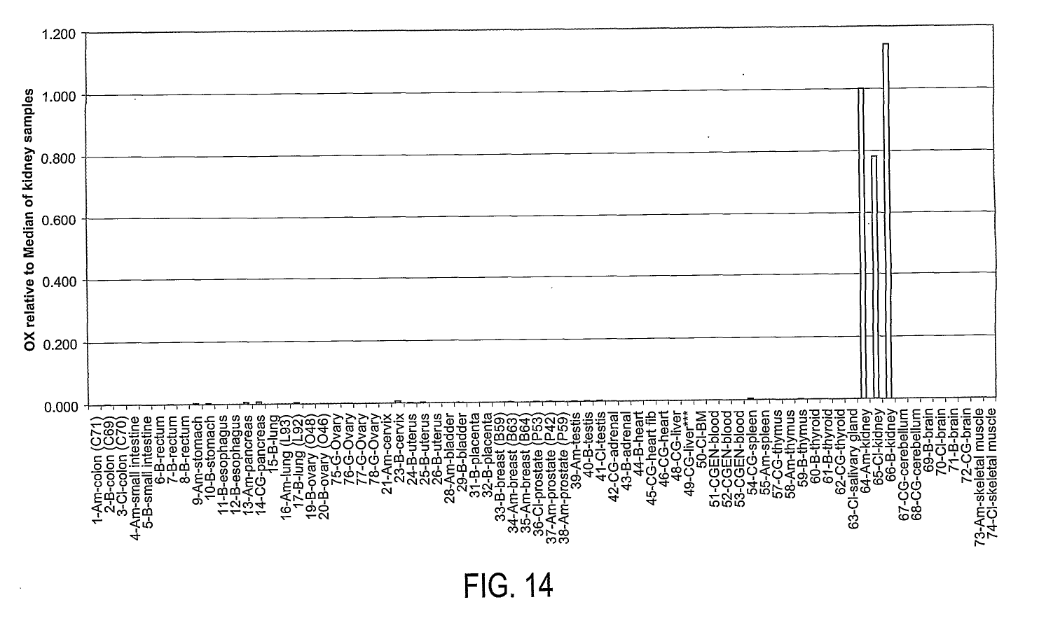

FIG. 14 is a histogram showing relative expression of the above-indicated Homo sapiens uromodulin (uromucoid, Tamm-Horsfall glycoprotein) (UMOD) transcripts in kidney tissue samples as opposed to other tissues.

As is evident from FIG. 14, the expression of Homo sapiens uromodulin (uromucoid, Tamm-Horsfall glycoprotein) (UMOD) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: HUMUMOD_seg56F (SEQ ID NO:253) forward primer; and HUMUMOD_seg56R (SEQ ID NO:254) reverse primer.

The present invention also preferably encompasses any amplicon obtained through the use of any suitable primer pair; for example, for the above experiment, the following amplicon was obtained as a non-limiting illustrative example only of a suitable amplicon: HUMUMOD_seg56 (SEQ ID NO:255).

Forward Primer (HUMUMOD_seg56F (SEQ ID NO: 253):
GTGGCCAGAGAGGGTCCCTA
Reverse Primer (HUMUMOD_seg56R (SEQ ID NO: 254):
CCTGTGGGTGGCAGTTGAC
Amplicon (HUMUMOD_seg56 (SEQ ID NO: 255):
TGGCCAGAGAGGGTCCCTAGGGCCCCTAGATGGTTCTAACCCCAAACCCC
TTAACCATGAGCTTCCCTGTCAACTGCCACCCACAGG

Expression of Homo sapiens uromodulin (uromucoid, Tamm-Horsfall glycoprotein) (UMOD) HUMUMOD transcripts which are detectable by amplicon as depicted in sequence name HUMUMOD_seg61WT (SEQ ID NO:258) specifically in kidney tissue

Expression of Homo sapiens uromodulin (uromucoid, Tamm-Horsfall glycoprotein) (UMOD) transcripts detectable by or according to seg61WT-HUMUMOD_seg61WT (SEQ ID NO:258) amplicon and primers HUMUMOD_seg61WTF (SEQ ID NO:256) and HUMUMOD_seg61WTR (SEQ ID NO:257) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by the median of the quantities of the kidney samples (sample numbers 64, 65 and 66, Table 1_{—}5 above), to obtain a value of relative expression of each sample relative to median of the kidney samples.

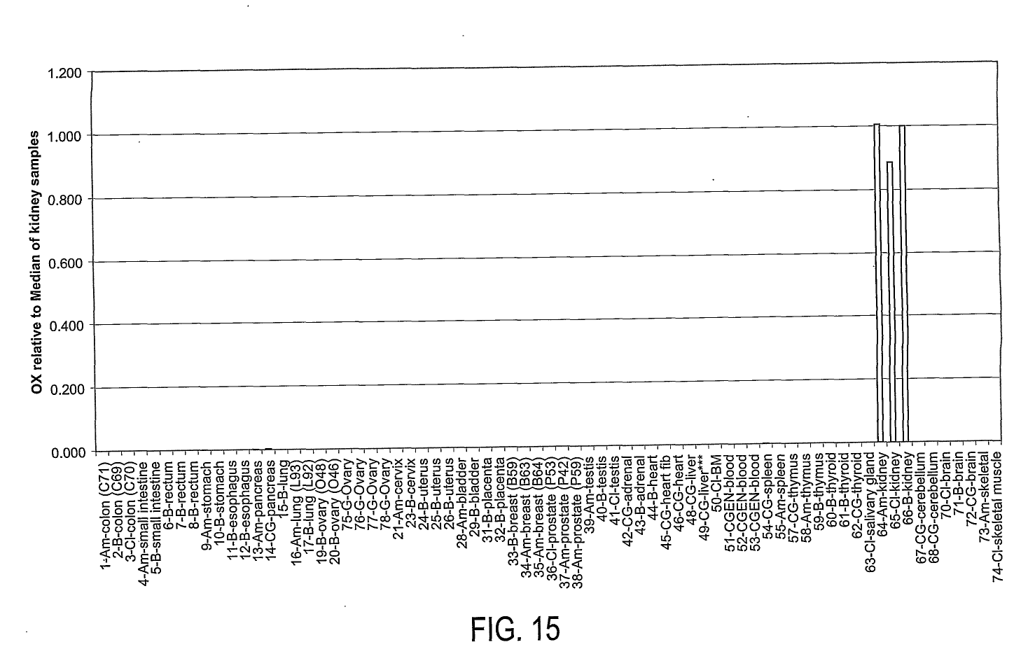

FIG. 15 is a histogram showing relative expression of the above-indicated Homo sapiens uromodulin (uromucoid, Tamm-Horsfall glycoprotein) (UMOD) transcripts in kidney tissue samples as opposed to other tissues.

As is evident from FIG. 15, the expression of Homo sapiens Homo sapiens uromodulin (uromucoid, Tamm-Horsfall glycoprotein) (UMOD) transcripts detectable by the above amplicon in kidney tissue samples was significantly higher than in all the other samples.

Primer pairs are also optionally and preferably encompassed within the present invention; for example, for the above experiment, the following primer pair was used as a non-limiting illustrative example only of a suitable primer pair: HUMUMOD_seg61WTF (SEQ ID NO:256) forward primer; and HUMUMOD_seg61WTR (SEQ ID NO:257) reverse primer.

The present invention also preferably encompasses any amplicon obtained through the use of any suitable primer pair; for example, for the above experiment, the following amplicon was obtained as a non-limiting illustrative example only of a suitable amplicon: HUMUMOD_seg61WT (SEQ ID NO:258).

Forward Primer (HUMUMOD_seg61WTF (SEQ ID NO: 256):
CTACCAAGGCTCCTCCGTGA
Reverse Primer (HUMUMOD_seg61WTR (SEQ ID NO: 257):
TGGTCATGAGCAGTGCAAATC
Amplicon (HUMUMOD_seg61WT (SEQ ID NO: 258):
CTACCAAGGCTCCTCCGTGACACTGTCCACTGAGGCTTTTCTCTACGTGG
GCACCATGTTGGATGGGGGCGACCTGTCCCGATTTGCACTGCTCATGACC
A

### Description for Cluster HSCP2

Cluster HSCP2 features 13 transcript(s) and 36 segment(s) of interest, the names for which are given in Tables 117 and 118, respectively. The selected protein variants are given in table 119.

TABLE 117
Transcripts of interest
Transcript Name

HSCP2_T5 (SEQ ID NO: 259)
HSCP2_T6 (SEQ ID NO: 260)
HSCP2_T7 (SEQ ID NO: 261)
HSCP2_T9 (SEQ ID NO: 262)
HSCP2_T11 (SEQ ID NO: 263)
HSCP2_T18 (SEQ ID NO: 264)
HSCP2_T19 (SEQ ID NO: 265)
HSCP2_T20 (SEQ ID NO: 266)
HSCP2_T21 (SEQ ID NO: 267)
HSCP2_T23 (SEQ ID NO: 268)
HSCP2_T25 (SEQ ID NO: 269)
HSCP2_T28 (SEQ ID NO: 270)
HSCP2_T29 (SEQ ID NO: 271)

TABLE 118
Segments of interest
Segment Name

HSCP2_N0 (SEQ ID NO: 272)
HSCP2_N3 (SEQ ID NO: 273)
HSCP2_N6 (SEQ ID NO: 274)
HSCP2_N10 (SEQ ID NO: 275)
HSCP2_N14 (SEQ ID NO: 276)
HSCP2_N18 (SEQ ID NO: 277)
HSCP2_N20 (SEQ ID NO: 278)
HSCP2_N23 (SEQ ID NO: 279)
HSCP2_N27 (SEQ ID NO: 280)
HSCP2_N29 (SEQ ID NO: 281)
HSCP2_N30 (SEQ ID NO: 282)
HSCP2_N32 (SEQ ID NO: 283)
HSCP2_N36 (SEQ ID NO: 284)
HSCP2_N41 (SEQ ID NO: 285)
HSCP2_N46 (SEQ ID NO: 286)
HSCP2_N52 (SEQ ID NO: 287)
HSCP2_N54 (SEQ ID NO: 288)
HSCP2_N65 (SEQ ID NO: 289)
HSCP2_N66 (SEQ ID NO: 290)
HSCP2_N4 (SEQ ID NO: 291)
HSCP2_N7 (SEQ ID NO: 292)
HSCP2_N13 (SEQ ID NO: 293)
HSCP2_N15 (SEQ ID NO: 294)
HSCP2_N17 (SEQ ID NO: 295)
HSCP2_N24 (SEQ ID NO: 296)
HSCP2_N35 (SEQ ID NO: 297)
HSCP2_N37 (SEQ ID NO: 298)
HSCP2_N44 (SEQ ID NO: 299)
HSCP2_N45 (SEQ ID NO: 300)
HSCP2_N49 (SEQ ID NO: 301)
HSCP2_N50 (SEQ ID NO: 302)
HSCP2_N55 (SEQ ID NO: 303)
HSCP2_N60 (SEQ ID NO: 304)
HSCP2_N61 (SEQ ID NO: 305)
HSCP2_N62 (SEQ ID NO: 306)
HSCP2_N63 (SEQ ID NO: 307)

TABLE 119
Proteins of interest

| Protein Name | Corresponding Transcript(s) |
|---|---|
| HSCP2_P3 (SEQ ID NO: 310) | HSCP2_T5 (SEQ ID NO: 259) |
| HSCP2_P4 (SEQ ID NO: 311) | HSCP2_T6 (SEQ ID NO: 260) |
| HSCP2_P5 (SEQ ID NO: 312) | HSCP2_T7 (SEQ ID NO: 261) |
| HSCP2_P6 (SEQ ID NO: 313) | HSCP2_T9 (SEQ ID NO: 262) |
| HSCP2_P8 (SEQ ID NO: 314) | HSCP2_T11 (SEQ ID NO: 263) |
| HSCP2_P15 (SEQ ID NO: 315) | HSCP2_T18 (SEQ ID NO: 264) |
| HSCP2_P16 (SEQ ID NO: 316) | HSCP2_T19 (SEQ ID NO: 265) |
| HSCP2_P18 (SEQ ID NO: 317) | HSCP2_T21 (SEQ ID NO: 267) |
| HSCP2_P21 (SEQ ID NO: 318) | HSCP2_T25 (SEQ ID NO: 269) |
| HSCP2_P23 (SEQ ID NO: 319) | HSCP2_T28 (SEQ ID NO: 270) |
| HSCP2_P24 (SEQ ID NO: 320) | HSCP2_129 (SEQ ID NO: 271) |
| HSCP2_P37 (SEQ ID NO: 321) | HSCP2_T20 (SEQ ID NO: 266) |
| HSCP2_P39 (SEQ ID NO: 322) | HSCP2_T23 (SEQ ID NO: 268) |

These sequences are variants of the known protein Ceruloplasmin precursor (SEQ ID NO:308) (SwissProt accession identifier CERU_HUMAN (SEQ ID NO:308); known also according to the synonyms EC 1.16.3.1; Ferroxidase), referred to herein as the previously known protein.

Protein Ceruloplasmin precursor (SEQ ID NO:308) is known or believed to have the following function(s): Ceruloplasmin is a blue, copper-binding (6-7 atoms per molecule) glycoprotein found in plasma. Four possible functions are ferroxidase activity, amine oxidase activity, copper transport and homeostasis, and superoxide dismutase activity. Known polymorphisms for this sequence are as shown in Table 120.

TABLE 120
Amino acid mutations for Known Protein

| SNP position(s) on | |
|---|---|
| amino acid sequence | Comment |
| 79 | T -> G. /FTId = VAR_001043 |
| 449 | L -> G. /FTId = VAR_001044 |
| 1060 | E -> EGEYP |

The following GO Annotation(s) apply to the previously known protein. The following annotation(s) were found: iron ion homeostasis, which are annotation(s) related to Biological Process; ferroxidase activity, which are annotation(s) related to Molecular Function; and extracellular space, which are annotation(s) related to Cellular Component.

The GO assignment relies on information from one or more of the SwissProt/TremBl Protein knowledgebase, available from <http://www.expasy.ch/sprot/>; or Locuslink, available from <http://www.ncbi.nlm.nih.gov/projects/LocusLink/>.

The variants HSCP2_P3 (SEQ ID NO:310), HSCP2_P6 (SEQ ID NO:313), HSCP2_P16 (SEQ ID NO:316) and HSCP2_P18 (SEQ ID NO:317) were previously disclosed by the inventors in published PCT application no WO 2005/116850 and U.S. application Ser. No. 11/050,857. The variant HSCP2_P23 (SEQ ID NO:319) was previously disclosed by the inventors in published PCT application no WO 2005/116850, WO2005/071058 and U.S. application Ser. Nos. 11/050,857, 11/043,860, 10/764,503. The variant HSCP2_P5 (SEQ ID NO:312) was previously disclosed by the inventors in published PCT application no WO2005/071058 and U.S. application Ser. No. 11/043,860. The variants HSCP2_P4 (SEQ ID NO:311), and HSCP2_P8 (SEQ ID NO:314) were previously disclosed by the inventors in published PCT application no WO 2005/116850, WO2004/096979, WO2005/071058 and U.S. application Ser. Nos. 11/050,857, 11/043,860, 10/242,799, 10/426,002, 10/764,503, hereby incorporated by reference as if fully set forth herein. The variants HSCP2_P3 (SEQ ID NO:310), HSCP2_P4 (SEQ ID NO:311), HSCP2_P5 (SEQ ID NO:312), HSCP2_P6 (SEQ ID NO:313), HSCP2_P8 (SEQ ID NO:314), HSCP2_P16 (SEQ ID NO:316), HSCP2_P18 (SEQ ID NO:317) and HSCP2_P23 (SEQ ID NO:319) have now been shown to have novel and surprising diagnostic uses as described herein for other variants of cluster HSCP2.

According to optional but preferred embodiments of the present invention, variants of this cluster according to the present invention (amino acid and/or nucleic acid sequences of HSCP2) may optionally have one or more of the following utilities, as described in greater detail below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

A non-limiting example of such a utility is the detection, diagnosis and/or determination of diagnosis of Wilson's disease. The method comprises detecting a HSCP2 variant, for example a variant protein, protein fragment, peptide, polynucleotide, polynucleotide fragment and/or oligonucleotide as described herein, optionally and preferably in a serum sample. The expression levels of the HSCP2 variant as determined in a patient can be further compared to those in a normal individual.

Involvement of the known Ceruloplasmin for the above utility is described with regard to U.S. Pat. No. 6,806,044, hereby incorporated by reference as if fully set forth herein.

Another non-limiting example of such a utility is the detection, diagnosis and/or determination of kidney disease detection and prognosis. The method comprises detecting a HSCP2 variant, for example a variant protein, protein fragment, peptide, polynucleotide, polynucleotide fragment and/or oligonucleotide as described herein, optionally and preferably in a serum sample. The expression levels of the HSCP2 variant as determined in a patient can be further compared to those in a normal individual.

Differential expression of the known Ceruloplasmin for the above utilities is described with regard to US Patent Application No. US20040029175, hereby incorporated by reference as if fully set forth herein.

Another non-limiting example of such a utility is the detection, diagnosis and/or determination of increased risk of preterm delivery. The method comprises detecting a HSCP2 variant, for example a variant protein, protein fragment, peptide, polynucleotide, polynucleotide fragment and/or oligonucleotide as described herein, optionally and preferably in a serum sample. The expression levels of the HSCP2 variant as determined in a patient can be further compared to those in a normal individual.

Differential expression of the known Ceruloplasmin for the above utilities is described with regard to U.S. Pat. No. 5,516,702, hereby incorporated by reference as if fully set forth herein, which describes a method for determining an early indication of increased risk of preterm delivery comprising a) obtaining a secretion sample from the vaginal cavity or the cervical canal from a pregnant patient after week 12 of pregnancy; and b) determining the level of ceruloplasmin.

Another non-limiting example of such a utility is the detection, diagnosis and/or determination of ovarian cancer. The method comprises detecting a HSCP2 variant, for example a variant protein, protein fragment, peptide, polynucleotide, polynucleotide fragment and/or oligonucleotide as described herein, optionally and preferably in a serum sample. The expression levels of the HSCP2 variant as determined in a patient can be further compared to those in a normal individual.

Differential expression of the known Ceruloplasmin for the above utility is described with regard to PCT Application No. WO 01/5177, hereby incorporated by reference as if fully set forth herein.

Another non-limiting example of such a utility is the detection, diagnosis and/or determination of breast cancer. The method comprises detecting a HSCP2 variant, for example a variant protein, protein fragment, peptide, polynucleotide, polynucleotide fragment and/or oligonucleotide as described herein, optionally and preferably in a serum sample. The expression levels of the HSCP2 variant as determined in a patient can be further compared to those in a normal individual.

Differential expression of the known Ceruloplasmin for the above utility is described with regard to PCT Application No. WO 05/043165, hereby incorporated by reference as if fully set forth herein.

Another non-limiting example of such a utility is the detection, diagnosis and/or determination of rheumatoid arthritis or osteoarthritis. The method comprises detecting a HSCP2 variant, for example a variant protein, protein fragment, peptide, polynucleotide, polynucleotide fragment and/or oligonucleotide as described herein, optionally and preferably in a serum sample. The expression levels of the HSCP2 variant as determined in a patient can be further compared to those in a normal individual.

Differential expression of the known Ceruloplasmin for the above utility is described with regard to PCT Application No. WO 04/092410, hereby incorporated by reference as if fully set forth herein.

According to optional but preferred embodiments of the present invention, variants of this cluster according to the present invention (amino acid and/or nucleic acid sequences of HSCP2) may optionally have one or more of the following utilities, as described with regard to Table 121 below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

TABLE 121
Table of Utilities for Variants of HSCP2, related to Ceruloplasmin:

| Utility | Reason | Reference |
|---|---|---|
| mutation are increase | | 15557511 |
| unsuitability to Parkinson | | |
| disease | | |
| marker for brain ischemic | HSCP2 is the direct | 16227395 |
| states in the serum and | transcriptional target of | |
| CSF | HIF1a, a TF activated by | |
| | hypoxia (15741220) | |
| marker for metastasis and | HSCP2 is the direct | 15111541 |
| poor prognosis in various | transcriptional target of | |
| solid tumor types | HIF1a, a TF activated by | |
| | hypoxia (15741220) | |
| over expression in the | | 12708132 |
| urine as a marker for type | | |
| II diabetes (alone or in | | |
| combination with | | |
| immunoglobulin G, | | |
| transferrin, and | | |
| orosomucoid) | | |
| over expression in the | | |
| serum as an indicator for | | |
| prostate and colon cancer | | |

According to other optional embodiments of the present invention, variants of this cluster according to the present invention (amino acid and/or nucleic acid sequences of HSCP2) may optionally have one or more of the following utilities, some of which are related to utilities described above. It should be noted that these utilities are optionally and preferably suitable for human and non-human animals as subjects, except where otherwise noted.

Table 122 below describes diagnostic utilities for the cluster HSCP2 that were found through microarrays, including the statistical significance thereof and a reference. One or more HSCP2 variants according to the present invention may optionally have one or more of these utilities.

TABLE 122

| Utility | Microarray source |
|---|---|
| 1. differential diagnosis of in | 1. GSE3325 |
| situ prostate cancer vs. | |
| metastasis (lower expression | |
| in metastasis) | |
| 2. identification of liver | 2. jp_atlas, med_all_avg |
| cellular damage, due to very | (internal database). |
| high expression in liver. | |

Other non-limiting exemplary utilities for HSCP2 variants according to the present invention are described in greater detail below and also with regard to the previous section on clinical utility.

Cluster HSCP2 can be used as a diagnostic marker according to overexpression of transcripts of this cluster in cancer. Expression of such transcripts in normal tissues is also given according to the previously described methods. The term “number” in the left hand column of the table and the numbers on the y-axis of the figure below refer to weighted expression of ESTs in each category, as “parts per million” (ratio of the expression of ESTs for a particular cluster to the expression of all ESTs in that category, according to parts per million).

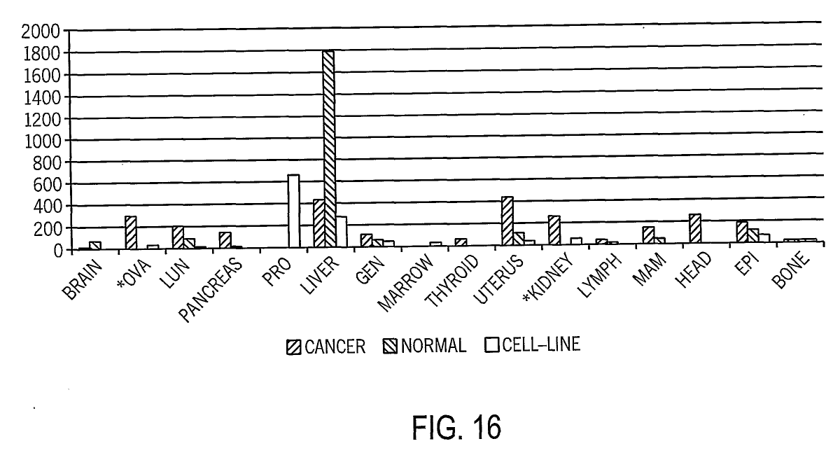

Overall, the following results were obtained as shown with regard to the histograms in FIG. 16 and Table 123. This cluster is overexpressed (at least at a minimum level) in the following pathological conditions: ovarian carcinoma and kidney malignant tumors.

TABLE 123
Normal tissue distribution

| Name of Tissue | Number |
|---|---|
| brain | 70 |
| ovary | 0 |
| lung | 93 |
| pancreas | 10 |
| prostate | 5 |
| liver | 1774 |
| general | 58 |
| bone marrow | 0 |
| Thyroid | 0 |
| uterus | 112 |
| kidney | 4 |
| lymph nodes | 18 |
| breast | 43 |
| head and neck | 0 |
| epithelial | 98 |
| bone | 9 |

TABLE 124
P values and ratios for expression in cancerous tissue

| Name of Tissue | P1 | P2 | SP1 | R3 | SP2 | R4 |
|---|---|---|---|---|---|---|
| brain | 7.7e−01 | 8.1e−01 | 1.0e+00 | 0.2 | 1.0e+00 | 0.1 |
| ovary | 1.6e−02 | 1.3e−02 | 1.1e−03 | 7.0 | 6.5e−03 | 5.2 |
| lung | 8.3e−01 | 8.9e−01 | 4.2e−02 | 1.1 | 5.5e−01 | 0.6 |
| pancreas | 2.1e−01 | 3.7e−01 | 1.2e−03 | 2.5 | 9.3e−03 | 1.9 |
| prostate | 9.7e−01 | 9.2e−01 | N/A | N/A | 9.3e−05 | 1.3 |
| liver | 2.9e−01 | 8.4e−01 | 1.0e+00 | 0.3 | 1.0e+00 | 0.1 |
| general | 5.3e−01 | 7.7e−01 | 9.1e−08 | 1.7 | 2.4e−03 | 1.2 |
| bone marrow | N/A | 7.1e−01 | N/A | N/A | 5.4e−01 | 1.9 |
| Thyroid | 5.7e−01 | 5.7e−01 | 6.8e−01 | 1.5 | 6.8e−01 | 1.5 |
| uterus | 1.9e−01 | 1.3e−01 | 6.2e−04 | 2.1 | 6.9e−02 | 1.3 |
| kidney | 2.5e−01 | 2.7e−01 | 2.6e−04 | 8.7 | 2.1e−03 | 6.2 |
| lymph nodes | 6.3e−01 | 8.3e−01 | 4.9e−01 | 1.7 | 8.2e−01 | 0.9 |
| breast | 3.2e−01 | 4.2e−01 | 2.3e−01 | 2.1 | 5.7e−01 | 1.3 |
| head and neck | 2.1e−01 | 3.3e−01 | 2.4e−01 | 3.5 | 5.7e−01 | 1.8 |
| epithelial | 3.1e−01 | 5.4e−01 | 2.6e−03 | 1.3 | 2.4e−01 | 0.9 |
| bone | 6.3e−01 | 8.3e−01 | N/A | N/A | 7.0e−01 | 1.2 |

As noted above, cluster HSCP2 features 13 transcript(s), which were listed in Table 117 above. These transcript(s) encode for protein(s) which are variant(s) of protein Ceruloplasmin precursor (SEQ ID NO:308). A description of each variant protein according to the present invention is now provided.

Variant protein HSCP2_P3 (SEQ ID NO:310) according to the present invention is encoded by transcript HSCP2_T5 (SEQ ID NO:259) One or more alignments to one or more previously published protein sequences are given in the alignment table on the attached CD-ROM. A brief description of the relationship of the variant protein according to the present invention to each such aligned protein is as follows:

1. Comparison report between HSCP2_P3 (SEQ ID NO:310) and CERU_HUMAN (SEQ ID NO:308):

A. An isolated chimeric polypeptide encoding for HSCP2_P3 (SEQ ID NO:310), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREFVVMFSVVDENFSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKHVRHYYIAAEEIIWNYAPSGIDIFTKENLTAPGSDSAVFFEQGTTRIGGSY KKLVYREYTDASFTNRKERGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTKDIFTGLIGPMKICKKGSLHANGRQKDVDKEFYLFPTVFDENESLLLEDNIRMFT TAPDQVDKEDEDFQESNKMHSMNGFMYGNQPGLTMCKGDSVVWYLFSAGNEADVHGI YFSGNTYLWRGERRDTANLFPQTSLTLHMWPDTEGTFNVECLTTDHYTGGMKQKYTVN QCRRQSEDSTFYLGERTYYIAAVEVEWDYSPQREWEKELHHLQEQNVSNAFLDKGEFYI GSKYKKVVYRQYTDSTFRVPVERKAEEEHLGILGPQLHADVGDKVKIIFKNMATRPYSIH AHGVQTESSTVTPTLPGETLTYVWKIPERSGAGTEDSACIPWAYYSTVDQVKDLYSGLIG PLIVCRRPYLKVFNPRRKLEFALLFLVFDENESWYLDDNIKTYSDHPEKVNKDDEEFIESN KMHAINGRMFGNLQGLTMHVGDEVNWYLMGMGNEIDLHTVHFHGHSFQYKHRGVYS SDVFDIFPGTYQTLEMFPRTPGIWLLHCHVTDHIHAGMETTYTVLQNE corresponding to amino acids 1-1060 of CERU_HUMAN (SEQ ID NO:308), which also corresponds to amino acids 1-1060 of HSCP2_P3 (SEQ ID NO:310), and a second amino acid sequence being at least 70%, optionally at least 80%, preferably at least 85%, more preferably at least 90% and most preferably at least 95% homologous to a polypeptide having the sequence GGTSM (SEQ ID NO:405) corresponding to amino acids 1061-1065 of HSCP2_P3 (SEQ ID NO:310), wherein said first amino acid sequence and second amino acid sequence are contiguous and in a sequential order.

B. An isolated polypeptide encoding for an edge portion of HSCP2_P3 (SEQ ID NO:310), comprising an amino acid sequence being at least 70%, optionally at least about 80%, preferably at least about 85%, more preferably at least about 90% and most preferably at least about 95% homologous to the sequence GGTSM (SEQ ID NO:405) of HSCP2_P3 (SEQ ID NO:310).

3. Comparison report between HSCP2_P3 (SEQ ID NO:310) and Q6NSB2_HUMAN (SEQ ID NO:309):

A. An isolated chimeric polypeptide encoding for HSCP2_P3 (SEQ ID NO:310), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P3 (SEQ ID NO:310), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKHRGVYSSDVFDIFPGTYQTLEMFPRTP GIWLLHCHVTDHIHAGMETTYTVLQNEGGTSM (SEQ ID NO:406) corresponding to amino acids 208-1065 of HSCP2_P3 (SEQ ID NO:310), wherein said first amino acid sequence and second amino acid sequence are contiguous and in a sequential order.

B. An isolated polypeptide encoding for an edge portion of HSCP2_P3 (SEQ ID NO:310), comprising an amino acid sequence being at least 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLIHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKHRGVYSSDVFDIFPGTYQTLEMFPRTP GIWLLHCHVTDHIHAGMETTYTVLQNEGGTSM (SEQ ID NO:406) of HSCP2_P3 (SEQ ID NO:310).

The localization of the variant protein was determined according to results from a number of different software programs and analyses, including analyses from SignalP and other specialized programs. The variant protein is believed to be secreted.

Variant protein HSCP2_P3 (SEQ ID NO:310) also has the following non-silent SNPs (Single Nucleotide Polymorphisms) as listed in Table 125, (given according to their position(s) on the amino acid sequence, with the alternative amino acid(s) listed).

TABLE 125
Amino acid mutations

| SNP position(s) on amino acid | |
|---|---|
| sequence | Alternative amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |
| 660 | F -> S |
| 676 | N -> |
| 727 | F -> S |
| 749 | R -> |
| 759 | Q -> |
| 927 | E -> K |

The glycosylation sites of variant protein HSCP2_P3 (SEQ ID NO:310), as compared to the known protein Ceruloplasmin precursor (SEQ ID NO:308), are described in Table 126 (given according to their position(s) on the amino acid sequence in the first column; the second column indicates whether the glycosylation site is present in the variant protein; and the last column indicates whether the position is different on the variant protein).

TABLE 126
Glycosylation site(s)

| Position(s) on known amino | | Position(s) on |
|---|---|---|
| acid sequence | Present in variant protein? | variant protein |
| 138 | Yes | 138 |
| 358 | Yes | 358 |
| 397 | Yes | 397 |
| 762 | Yes | 762 |

Variant protein HSCP2_P3 (SEQ ID NO:310) is encoded by the following transcript(s): HSCP2_T5 (SEQ ID NO:259), for which the sequence(s) is/are given at the end of the application. The coding portion of transcript HSCP2_T5 (SEQ ID NO:259) is shown in bold; this coding portion starts at position 251 and ends at position 3445. The transcript also has the following SNPs as listed in Table 127 (given according to their position on the nucleotide sequence, with the alternative nucleic acid listed).

TABLE 127
Nucleic acid SNPs

| SNP position(s) on nucleotide | |
|---|---|
| sequence | Alternative nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |
| 2140 | T -> C |
| 2200 | A -> C |
| 2229 | T -> C |
| 2277 | A -> |
| 2365 | C -> T |
| 2430 | T -> C |
| 2495 | A -> |
| 2527 | A -> |
| 2737 | A -> G |
| 2989 | A -> G |
| 3029 | G -> A |
| 3241 | T -> C |
| 3277 | A -> G |
| 5132 | C -> A |

Variant protein HSCP2_P4 (SEQ ID NO:311) according to the present invention is encoded by transcript HSCP2_T6 (SEQ ID NO:260) One or more alignments to one or more previously published protein sequences are given in the alignment table on the attached CD-ROM. A brief description of the relationship of the variant protein according to the present invention to each such aligned protein is as follows:

1. Comparison report between HSCP2_P4 (SEQ ID NO:311) and CERU_HUMAN (SEQ ID NO:308):

A. An isolated chimeric polypeptide encoding for HSCP2_P4 (SEQ ID NO:311), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREFVVMFSVVDENFSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKHVRHYYIAAEEIIWNYAPSGIDIFTKENLTAPGSDSAVFFEQGTTRIGGSY KKLVYREYTDASFTNRKERGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTDIFTGLIGPMKICKKGSLHANGRQKDVDKEFYLFPTVFDENESLLLEDNIRMFT TAPDQVDKEDEDFQESNKMHSMNGFMYGNQPGLTMCKGDSVVWYLFSAGNEADVHGI YFSGNTYLWRGERRDTANLFPQTSLTLHMWPDTEGTFNVECLTTDHYTGGMKQKYTVN QCRRQSEDSTFYLGERTYYIAAVEVEWDYSPQREWEKELHHLQEQ corresponding to amino acids 1-761 of CERU_HUMAN (SEQ ID NO:308), which also corresponds to amino acids 1-761 of HSCP2_P4 (SEQ ID NO:311), and a second amino acid K corresponding to amino acid 762 of HSCP2_P4 (SEQ ID NO:311), wherein said first amino acid sequence and second amino acid sequence are contiguous and in a sequential order.

3. Comparison report between HSCP2_P4 (SEQ ID NO:311) and Q6NSB2_HUMAN (SEQ ID NO:309):

A. An isolated chimeric polypeptide encoding for HSCP2_P4 (SEQ ID NO:311), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTPHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P4 (SEQ ID NO:311), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQK (SEQ ID NO:407) corresponding to amino acids 208-762 of HSCP2_P4 (SEQ ID NO:311), wherein said first amino acid sequence and second amino acid sequence are contiguous and in a sequential order.

B. An isolated polypeptide encoding for an edge portion of HSCP2_P4 (SEQ ID NO:311), comprising an amino acid sequence being at least 70%, optionally at least about 80%, preferably at least about 85%, more preferably at least about 90% and most preferably at least about 95% homologous to the sequence EKEKHIDREFVVMFSWDENFSWYLEDNIKTYCSEPEKVDKDNEDFQESNRMYSVNGYT FGSLPGLSMCAEDRVKWYLFGMGNEVDVHAAFPHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKRGPE EEHLGILGPVIWAEVGDTIRVTFNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHIHLQEQK (SEQ ID NO:407) of HSCP2_P4 (SEQ ID NO:311).

The localization of the variant protein was determined according to results from a number of different software programs and analyses, including analyses from SignalP and other specialized programs. The variant protein is believed to secreted.

Variant protein HSCP2_P4 (SEQ ID NO:311) also has the following non-silent SNPs (Single Nucleotide Polymorphisms) as listed in Table 128, (given according to their position(s) on the amino acid sequence, with the alternative amino acid(s) listed).

TABLE 128
Amino acid mutations

| SNP position(s) on amino acid | |
|---|---|
| sequence | Alternative amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |
| 660 | F -> S |
| 676 | N -> |
| 727 | F -> S |
| 749 | R -> |
| 759 | Q -> |

The glycosylation sites of variant protein HSCP2_P4 (SEQ ID NO:311), as compared to the known protein Ceruloplasmin precursor (SEQ ID NO:308), are described in Table 129 (given according to their position(s) on the amino acid sequence in the first column; the second column indicates whether the glycosylation site is present in the variant protein; and the last column indicates whether the position is different on the variant protein).

TABLE 129
Glycosylation site(s)

| Position(s) on known amino | | Position(s) on |
|---|---|---|
| acid sequence | Present in variant protein? | variant protein |
| 138 | Yes | 138 |
| 358 | Yes | 358 |
| 397 | Yes | 397 |
| 762 | No | |

Variant protein HSCP2_P4 (SEQ ID NO:311) is encoded by the transcript HSCP2_T6 (SEQ ID NO:260), for which the coding portion starts at position 251 and ends at position 2536. The transcript also has the following SNPs as listed in Table 130 (given according to their position on the nucleotide sequence, with the alternative nucleic acid listed).

TABLE 130
Nucleic acid SNPs

| SNP position(s) on nucleotide | |
|---|---|
| sequence | Alternative nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |
| 2140 | T -> C |
| 2200 | A -> C |
| 2229 | T -> C |
| 2277 | A -> |
| 2365 | C -> T |
| 2430 | T -> C |
| 2495 | A -> |
| 2527 | A -> |
| 2798 | A -> G |
| 3050 | A -> G |
| 3090 | G -> A |
| 3302 | T -> C |
| 3338 | A -> G |

Variant protein HSCP2_P5 (SEQ ID NO:312) according to the present invention is encoded by transcript HSCP2_T7 (SEQ ID NO:261) One or more alignments to one or more previously published protein sequences are given in the alignment table on the attached CD-ROM. A brief description of the relationship of the variant protein according to the present invention to each such aligned protein is as follows:

1. Comparison report between HSCP2_P5 (SEQ ID NO:312) and CERU_HUMAN (SEQ ID NO:308):

A. An isolated chimeric polypeptide encoding for HSCP2_P5 (SEQ ID NO:312), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREFVVMFSVVDENFSWYLEDNIKTYCSEP EKVDKDNEDFQESNRMYSVNGYTFGSLPGLSMCAEDRVKWYLFGMGNEVDVHAAFFH GQALTKNYRIDTINLFPATLFDAYMVAQNPGEWMLSCQNLNHLKAGLQAFFQVQECN KSSSKDNIRGKHVRHYYIAAEEIIWNYAPSGIDIFTKENLTAPGSDSAVFFEQGTTRIGGSY KKLVYREYTDASFTNRKERGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTKDIFTGLIGPMKICKKGSLHANGRQKDVDKEFYLFPTVFDENESLLLEDNIRMFT TAPDQVDKEDEDFQESNKMHSMNGFMYGNQPGLTMCKGDSVVWYLFSAGNEADVHGI YFSGNTYLWRGERRDTANLFPQTSLTLHMWPDTEGTFNVECLTTDHYTGGMKQKYTVN QCRRQSEDSTFYLGERTYYIAAVEVEWDYSPQREWEKLHBLQEQNVSNAFLDKGEFYI GSKYKKWYRQYTDSTFRVPVERKAEEEHLGILGPQLHADVGDKVKIIFKNMATRPYSIH AHGVQTESSTVTPTLPGETLTYVWKIPERSGAGTEDSACIPWAYYSTVDQVKDLYSGLIG PLIVCRRPYLKVFNPRRKLEFALLFLVFDENESWYLDDNIKTYSDHPEKVNKDDEEFIESN KMHAINGRMFGNLQGLTMIIVGDEVNWYLMGMGNEIDLHTVHFHGHSFQYKHRGVYS SDVFDIFPGTYQTLEMFPRTPGIWLLHCHVTDHIHAGMETTYTVLQNE corresponding to amino acids 1-1060 of CERU_HUMAN (SEQ ID NO:308), which also corresponds to amino acids 1-1060 of HSCP2_P5 (SEQ ID NO:312), a second amino acid sequence being at least 70%, optionally at least 80%, preferably at least 85%, more preferably at least 90% and most preferably at least 95% homologous to a polypeptide having the sequence GEYP (SEQ ID NO:408) corresponding to amino acids 1061-1064 of HSCP2_P5 (SEQ ID NO:312), and a third amino acid sequence being at least 90% homologous to DTKSG corresponding to amino acids 1061-1065 of CERU_HUMAN (SEQ ID NO:308), which also corresponds to amino acids 1065-1069 of HSCP2_P5 (SEQ ID NO:312), wherein said first amino acid sequence, second amino acid sequence and third amino acid sequence are contiguous and in a sequential order.

B. An isolated polypeptide encoding for an edge portion of HSCP2_P5 (SEQ ID NO:312), comprising an amino acid sequence being at least 70%, optionally at least about 80%, preferably at least about 85%, more preferably at least about 90% and most preferably at least about 95% homologous to the sequence GEYP (SEQ ID NO:408) of HSCP2_P5 (SEQ ID NO:312).

3. Comparison report between HSCP2_P5 (SEQ ID NO:312) and Q6NSB2_HUMAN (SEQ ID NO:309):

A. An isolated chimeric polypeptide encoding for HSCP2_P5 (SEQ ID NO:312), comprising a first amino acid sequence being at least 90% homologous to MKILILGIFLFLCSTPAWAKEKHMGIIETTWDYASDHGEKKLISVDTEHSNRYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P5 (SEQ ID NO:312), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMPTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSPQYKHRGVYSSDVFDIFPGTYQTLEMFPRTP GIWLLHCHVTDHIHAGMETTYTVLQNEGEYPDTKSG (SEQ ID NO:409) corresponding to amino acids 208-1069 of HSCP2_P5 (SEQ ID NO:312), wherein said first amino acid sequence and second amino acid sequence are contiguous and in a sequential order.

B. An isolated polypeptide encoding for an edge portion of HSCP2_P5 (SEQ ID NO:312), comprising an amino acid sequence being at least 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIMAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDILHTVHFHGHSFQYKHRGVYSSDVFDIFPGTYQTLEMFPRTP GIWLLHCHVTDHIHAGMETTYTVLQNEGEYPDTKSG (SEQ ID NO:409) of HSCP2_P5 (SEQ ID NO:312).

The localization of the variant protein was determined according to results from a number of different software programs and analyses, including analyses from SignalP and other specialized programs. The variant protein is believed to be secreted.

Variant protein HSCP2_P5 (SEQ ID NO:312) also has the following non-silent SNPs (Single Nucleotide Polymorphisms) as listed in Table 131, (given according to their position(s) on the amino acid sequence, with the alternative amino acid(s) listed).

TABLE 131
Amino acid mutations

| SNP position(s) on amino acid | |
|---|---|
| sequence | Alternative amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |
| 660 | F -> S |
| 676 | N -> |
| 727 | F -> S |
| 749 | R -> |
| 759 | Q -> |
| 927 | E -> K |

The glycosylation sites of variant protein HSCP2_P5 (SEQ ID NO:312), as compared to the known protein Ceruloplasmin precursor (SEQ ID NO:308), are described in Table 132 (given according to their position(s) on the amino acid sequence in the first column; the second column indicates whether the glycosylation site is present in the variant protein; and the last column indicates whether the position is different on the variant protein).

TABLE 132
Glycosylation site(s)

| Position(s) on known amino | | Position(s) on |
|---|---|---|
| acid sequence | Present in variant protein? | variant protein |
| 138 | Yes | 138 |
| 358 | Yes | 358 |
| 397 | Yes | 397 |
| 762 | Yes | 762 |

Variant protein HSCP2_P5 (SEQ ID NO:312) is encoded by the transcript HSCP2_T7 (SEQ ID NO:261), for which the coding portion starts at position 251 and ends at position 3457. The transcript also has the following SNPs as listed in Table 133 (given according to their position on the nucleotide sequence, with the alternative nucleic acid listed).

TABLE 133
Nucleic acid SNPs

| SNP position(s) | |
|---|---|
| on nucleotide | Alternative |
| sequence | nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |
| 2140 | T -> C |
| 2200 | A -> C |
| 2229 | T -> C |
| 2277 | A -> |
| 2365 | C -> T |
| 2430 | T -> C |
| 2495 | A -> |
| 2527 | A -> |
| 2737 | A -> G |
| 2989 | A -> G |
| 3029 | G -> A |
| 3241 | T -> C |
| 3277 | A -> G |
| 3888 | A -> C |
| 3906 | A -> G |
| 3954 | T -> |
| 3969 | A -> C |
| 4055 | A -> T |
| 4105 | A -> G |
| 4229 | G -> A |
| 4427 | G -> A |
| 4617 | C -> T |

Variant protein HSCP2_P6 (SEQ ID NO:313) according to the present invention is encoded by transcript HSCP2_T9 (SEQ ID NO:262) One or more alignments to one or more previously published protein sequences are given in the alignment table on the attached CD-ROM. A brief description of the relationship of the variant protein according to the present invention to each such aligned protein is as follows:

1. Comparison report between HSCP2_P6 (SEQ ID NO:313) and CERU_HUMAN (SEQ ID NO:308):

A. An isolated chimeric polypeptide encoding for HSCP2_P6 (SEQ ID NO:313), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREFVVMFSVVDENFSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKHVRHYYIAAEEIIWNYAPSGIDIFTKENLTAPGSDSAVFFEQGTTRIGGSY KKLVYREYTDASFTNRKERGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTKDIFTGLIGPMKICKKGSLHANGRQKDVDKEFYLFPTVFDENESLLLEDNIRMFT TAPDQVDKEDEDFQESNKMHSMNGFMYGNQPGLTMCKGDSVVWYLFSAGNEADVHGI YFSGNTYLWRGERRDTANLFPQTSLTLHMWPDTEGTFNVECLTTDHYTGGMKQKYTVN QCRRQSEDSTFYLGERTYYIAAVEVEWDYSPQREWEKELHHLQEQNVSNAFLDKGEFYI GSKYKKVVYRQYTDSTFRVPVERKAEEEHLGILGPQLHADVGDKVKIIFKNMATRPYSIH AHGVQTESSTVTPTLPGETLTYVWKIPERSGAGTEDSACIPWAYYSTVDQVKDLYSGLIG PLIVCRRPYLKVFNPRRKLEFALLFLVFDENESWYLDDNIKTYSDHPEKVNKDDEEFIESN KMHAINGRMFGNLQGLTMHVGDEVNWYLMGMGNEIDLHTVHFHGHSFQYK corresponding to amino acids 1-1006 of CERU_HUMAN (SEQ ID NO:308), which also corresponds to amino acids 1-1006 of HSCP2_P6 (SEQ ID NO:313), and a second amino acid sequence being at least 70%, optionally at least 80%, preferably at least 85%, more preferably at least 90% and most preferably at least 95% homologous to a polypeptide having the sequence GSL (SEQ ID NO:411) corresponding to amino acids 1007-1009 of HSCP2_P6 (SEQ ID NO:313), wherein said first amino acid sequence and second amino acid sequence are contiguous and in a sequential order.

B. An isolated polypeptide encoding for an edge portion of HSCP2_P6 (SEQ ID NO:313), comprising an amino acid sequence being at least 70%, optionally at least about 80%, preferably at least about 85%, more preferably at least about 90% and most preferably at least about 95% homologous to the sequence GSL (SEQ ID NO:411) of HSCP2_P6 (SEQ ID NO:313).

3. Comparison report between HSCP2_P6 (SEQ ID NO:313) and Q6NSB2_HUMAN (SEQ ID NO:309):

A. An isolated chimeric polypeptide encoding for HSCP2_P6 (SEQ ID NO:313), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P6 (SEQ ID NO:313), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFIINKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKGSL (SEQ ID NO:410) corresponding to amino acids 208-1009 of HSCP2_P6 (SEQ ID NO:313), wherein said first amino acid sequence and second amino acid sequence are contiguous and in a sequential order.

B. An isolated polypeptide encoding for an edge portion of HSCP2_P6 (SEQ ID NO:313), comprising an amino acid sequence being at least 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKVVYRQYTDSTFRVP VERKAEEEHLGILGPQLHADVGDKVKIIFKNMATRPYSIHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKGSL (SEQ ID NO:410) of HSCP2_P6 (SEQ ID NO:313).

The localization of the variant protein was determined according to results from a number of different software programs and analyses, including analyses from SignalP and other specialized programs. The variant protein is believed to be secreted.

Variant protein HSCP2_P6 (SEQ ID NO:313) also has the following non-silent SNPs (Single Nucleotide Polymorphisms) as listed in Table 134, (given according to their position(s) on the amino acid sequence, with the alternative amino acid(s) listedn).

TABLE 134
Amino acid mutations

| SNP position(s) | |
|---|---|
| on amino acid | Alternative |
| sequence | amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |
| 660 | F -> S |
| 676 | N -> |
| 727 | F -> S |
| 749 | R -> |
| 759 | Q -> |
| 927 | E -> K |
| 1008 | S -> G |

The glycosylation sites of variant protein HSCP2_P6 (SEQ ID NO:313), as compared to the known protein Ceruloplasmin precursor (SEQ ID NO:308), are described in Table 135 (given according to their position(s) on the amino acid sequence in the first column; the second column indicates whether the glycosylation site is present in the variant protein; and the last column indicates whether the position is different on the variant protein).

TABLE 135
Glycosylation site(s)

| Position(s) on | | |
|---|---|---|
| known amino | Present in | Position(s) on |
| acid sequence | variant protein? | variant protein |
| 138 | Yes | 138 |
| 358 | Yes | 358 |
| 397 | Yes | 397 |
| 762 | Yes | 762 |

Variant protein HSCP2_P6 (SEQ ID NO:313) is encoded by the transcript HSCP2_T9 (SEQ ID NO:262), for which the coding portion starts at position 251 and ends at position 3277. The transcript also has the following SNPs as listed in Table 136 (given according to their position on the nucleotide sequence, with the alternative nucleic acid listed).

TABLE 136
Nucleic acid SNPs

| SNP position(s) | |
|---|---|
| on nucleotide | Alternative |
| sequence | nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |
| 2140 | T -> C |
| 2200 | A -> C |
| 2229 | T -> C |
| 2277 | A -> |
| 2365 | C -> T |
| 2430 | T -> C |
| 2495 | A -> |
| 2527 | A -> |
| 2737 | A -> G |
| 2989 | A -> G |
| 3029 | G -> A |
| 3241 | T -> C |
| 3272 | A -> G |

Variant protein HSCP2_P8 (SEQ ID NO:314) according to the present invention is encoded by transcript HSCP2_T11 (SEQ ID NO:263) One or more alignments to one or more previously published protein sequences are given in the alignment table on the attached CD-ROM. A brief description of the relationship of the variant protein according to the present invention to each such aligned protein is as follows:

1. Comparison report between HSCP2_P8 (SEQ ID NO:314) and CERU_HUMAN (SEQ ID NO:308):

A. An isolated chimeric polypeptide encoding for HSCP2_P8 (SEQ ID NO:314), comprising a first amino acid sequence being at least 90% homologous to MKILILGIFLFLCSTPAWAKEKYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEHIDREFVVMFSVVDENFSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKHVRHYYIAAEEIIWNYAPSGIDIFTKENLTAPGSDSAVFFEQGTTRIGGSY KKLVYREYTDASF RGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTKDIFTGLIGPMKICKKGSLHANGRQKDVDKEFYLFPTVFDENESLLLEDNIRMFT TAPDQVDKEDEDFQESNKMHSMNGFMYGNQPGLTMCKGDSVVWYLFSAGNEADVHGI YFSGNTYLWRGERRDTANLFPQTSLTLHMWPDTEGTFNVECLTTDHYTGGMKQKYTVN QCRRQSEDSTFYLGERTYYIAAVEVEWDYSPQREWEKELHHLQEQNVSNAFLDKGEFYI GSKYKKVVYRQYTDSTFRVPVERKAEEEHLGILGPQLHADVGDKVKIIFKNMATRPYSIH AHGVQTESSTVTPTLPGETLTYVWKIPERSGAGTEDSACIPWAYYSTVDQVKDLYSGLIG PLIVCRRPYLKVFNPRRKLEFALLFLVFDENESWYLDDNIKTYSDHPEKVNKDDEEFIESN KMHAINGRMFGNLQGLTMHVGDEVNWYLMGMGNEIDLHTVHFHGHSFQYK corresponding to amino acids 1-1006 of CERU_HUMAN (SEQ ID NO:308), which also corresponds to amino acids 1-1006 of HSCP2_P8 (SEQ ID NO:314), and a second amino acid sequence being at least 70%, optionally at least 80%, preferably at least 85%, more preferably at least 90% and most preferably at least 95% homologous to a polypeptide having the sequence KCFQEHLEFGYSTAM (SEQ ID NO:412) corresponding to amino acids 1007-1021 of HSCP2_P8 (SEQ ID NO:314), wherein said first amino acid sequence and second amino acid sequence are contiguous and in a sequential order.

B. An isolated polypeptide encoding for an edge portion of HSCP2_P8 (SEQ ID NO:314), comprising an amino acid sequence being at least 70%, optionally at least about 80%, preferably at least about 85%, more preferably at least about 90% and most preferably at least about 95% homologous to the sequence KCFQEHLEFGYSTAM (SEQ ID NO:412) of HSCP2_P8 (SEQ ID NO:314).

3. Comparison report between HSCP2_P8 (SEQ ID NO:314) and Q6NSB2_HUMAN (SEQ ID NO:309):

A. An isolated chimeric polypeptide encoding for HSCP2_P8 (SEQ ID NO:314), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P8 (SEQ ID NO:314), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFIINKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHIIFHGHSFQYKKCFQEHLEFGYSTAM (SEQ ID NO:413) corresponding to amino acids 208-1021 of HSCP2_P8 (SEQ ID NO:314), wherein said first amino acid sequence and second amino acid sequence are contiguous and in a sequential order.

B. An isolated polypeptide encoding for an edge portion of HSCP2_P8 (SEQ ID NO:314), comprising an amino acid sequence being at least 70%, optionally at least about 80%, preferably at least about 85%, more preferably at least about 90% and most preferably at least about 95% homologous to the sequence EKEKHDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKKCFQEHLEFGYSTAM (SEQ ID NO:413) of HSCP2_P8 (SEQ ID NO:314).

The localization of the variant protein was determined according to results from a number of different software programs and analyses, including analyses from SignalP and other specialized programs. The variant protein is believed to be secreted.

Variant protein HSCP2_P8 (SEQ ID NO:314) also has the following non-silent SNPs (Single Nucleotide Polymorphisms) as listed in Table 137, (given according to their position(s) on the amino acid sequence, with the alternative amino acid(s) listed).

TABLE 137
Amino acid mutations

| SNP position(s) | |
|---|---|
| on amino acid | Alternative |
| sequence | amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |
| 660 | F -> S |
| 676 | N -> |
| 727 | F -> S |
| 749 | R -> |
| 759 | Q -> |
| 927 | E -> K |

The glycosylation sites of variant protein HSCP2_P8 (SEQ ID NO:314), as compared to the known protein Ceruloplasmin precursor (SEQ ID NO:308), are described in Table 138 (given according to their position(s) on the amino acid sequence in the first column; the second column indicates whether the glycosylation site is present in the variant protein; and the last column indicates whether the position is different on the variant protein).

TABLE 138
Glycosylation site(s)

| Position(s) on | | |
|---|---|---|
| known amino | Present in | Position(s) on |
| acid sequence | variant protein? | variant protein |
| 138 | Yes | 138 |
| 358 | Yes | 358 |
| 397 | Yes | 397 |
| 762 | Yes | 762 |

Variant protein HSCP2_P8 (SEQ ID NO:314) is encoded by the transcript HSCP2_T11 (SEQ ID NO:263), for which the coding portion starts at position 251 and ends at position 3313. The transcript also has the following SNPs as listed in Table 139 (given according to their position on the nucleotide sequence, with the alternative nucleic acid listed; the last column indicates whether the SNP is known or not; the presence of known SNPs in variant protein HSCP2_P8 (SEQ ID NO:314) sequence provides support for the deduced sequence of this variant protein according to the present invention).

TABLE 139
Nucleic acid SNPs

| SNP position(s) | |
|---|---|
| on nucleotide | Alternative |
| sequence | nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |
| 2140 | T -> C |
| 2200 | A -> C |
| 2229 | T -> C |
| 2277 | A -> |
| 2365 | C -> T |
| 2430 | T -> C |
| 2495 | A -> |
| 2527 | A -> |
| 2737 | A -> G |
| 2989 | A -> G |
| 3029 | G -> A |
| 3241 | T -> C |

Variant protein HSCP2_P15 (SEQ ID NO:315) according to the present invention is encoded by transcript HSCP2_T18 (SEQ ID NO:264) One or more alignments to one or more previously published protein sequences are given in the alignment table oh the attached CD-ROM. A brief description of the relationship of the variant protein according to the present invention to each such aligned protein is as follows:

1. Comparison report between HSCP2_P15 (SEQ ID NO:315) and CERU_HUMAN (SEQ ID NO:308):

A. An isolated chimeric polypeptide encoding for HSCP2_P15 (SEQ ID NO:315), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREFVVMFSVVDENSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKIIVRHYYIAAEEIIWNYAPSGIDIFTKENLTAPGSDSAVFFEQGTTRIGGSY KKLVYREYTDASFTNRKERGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTKDIFTGLIGPMKICKKGSLHANGRQKDVDKEFYLFPTVFDENESLLLEDNIRMFT TAPDQVDKEDEDFQESNKMHSMNGFMYGNQPGLTMCKGDSVVWYLFSAGNEADVHGI YFSGNTYLWRGERRDTANLFPQTSLTLHMWPDTEGTFNVECLTTDHYTGGMKQKYTVN QCRRQSEDSTFYLGERTYYIAAVEVEWDYSPQREWEKELHHLQEQNVSNAFLDKGEFYI GSKYKKVVYRQYTDSTFRVPVERKAEEEHLGILGPQLHADVGDKVKIIFKNATRPYSIH AHGVQTESSTVTPTLPGETLTYVWKIPERSGAGTEDSACIPWAYYSTVDQVKDLYSGLIG PLIVCRRPYLKVFNPRRKLEFALLFLVFDENESWYLDDNIKTYSDHPEKVNKDDEEFIESN KMHAINGRMFGNLQGLTMHVGDEVNWYLMGMGNEIDLHTVHIFHGHSFQYK corresponding to amino acids 1-1006 of CERU_HUMAN (SEQ ID NO:308), which also corresponds to amino acids 1-1006 of HSCP2_P15 (SEQ ID NO:315), and a second amino acid sequence being at least 70%, optionally at least 80%, preferably at least 85%, more preferably at least 90% and most preferably at least 95% homologous to a polypeptide having the sequence VRAIHGNYSCSV (SEQ ID NO:414) corresponding to amino acids 1007-1018 of HSCP2_P15 (SEQ ID NO:315), wherein said first amino acid sequence and second amino acid sequence are contiguous and in a sequential order.

B. An isolated polypeptide encoding for an edge portion of HSCP2_P15 (SEQ ID NO:315), comprising an amino acid sequence being at least 70%, optionally at least about 80%, preferably at least about 85%, more preferably at least about 90% and most preferably at least about 95% homologous to the sequence VRAIHGNYSCSV (SEQ ID NO:414) of HSCP2_P15 (SEQ ID NO:315).

3. Comparison report between HSCP2_P15 (SEQ ID NO:315) and Q6NSB2_HUMAN (SEQ ID NO:309):

A. An isolated chimeric polypeptide encoding for HSCP2_P15 (SEQ ID NO:315), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P15 (SEQ ID NO:315), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKM YYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKVRAIHGNYSCSV (SEQ ID NO:415) corresponding to amino acids 208-1018 of HSCP2_P15 (SEQ ID NO:315), wherein said first amino acid sequence and second amino acid sequence are contiguous and in a sequential order.

B. An isolated polypeptide encoding for an edge portion of HSCP2_P15 (SEQ ID NO:315), comprising an amino acid sequence being at least 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDIIYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGETLT YVWKIPERSGAGTEDSACIPWAYYSTVDQVKDLYSGLIGPLIVCRRPYLKVFNPRRKLEF ALLFLVFDENESWYLDDNIKTYSDHPEKVNKDDEEFIESNKMHAINGRMFGNLQGLTMH VGDEVNWYLMGMGNEIDLHTVHFHGHSFQYKVRAIHGNYSCSV (SEQ ID NO:415) of HSCP2_P15 (SEQ ID NO:315).

The localization of the variant protein was determined according to results from a number of different software programs and analyses, including analyses from SignalP and other specialized programs. The variant protein is believed to be secreted.

Variant protein HSCP2_P15 (SEQ ID NO:315) also has the following non-silent SNPs (Single Nucleotide Polymorphisms) as listed in Table 140, (given according to their position(s) on the amino acid sequence, with the alternative amino acid(s) listed).

TABLE 140
Amino acid mutations

| SNP position(s) | |
|---|---|
| on amino acid | Alternative |
| sequence | amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |
| 660 | F -> S |
| 676 | N -> |
| 727 | F -> S |
| 749 | R -> |
| 759 | Q -> |
| 927 | E -> K |

The glycosylation sites of variant protein HSCP2_P15 (SEQ ID NO:315), as compared to the known protein Ceruloplasmin precursor (SEQ ID NO:308), are described in Table 141 (given according to their position(s) on the amino acid sequence in the first column; the second column indicates whether the glycosylation site is present in the variant protein; and the last column indicates whether the position is different on the variant protein).

TABLE 141
Glycosylation site(s)

| Position(s) on | | |
|---|---|---|
| known amino | Present in | Position(s) on |
| acid sequence | variant protein? | variant protein |
| 138 | Yes | 138 |
| 358 | Yes | 358 |
| 397 | Yes | 397 |
| 762 | Yes | 762 |

Variant protein HSCP2_P15 (SEQ ID NO:315) is encoded by the transcript HSCP2_T18 (SEQ ID NO:264), for which the coding portion starts at position 251 and ends at position 3304. The transcript also has the following SNPs as listed in Table 142 (given according to their position on the nucleotide sequence, with the alternative nucleic acid listed).

TABLE 142
Nucleic acid SNPs

| SNP position(s) | |
|---|---|
| on nucleotide | Alternative |
| sequence | nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |
| 2140 | T -> C |
| 2200 | A -> C |
| 2229 | T -> C |
| 2277 | A -> |
| 2365 | C -> T |
| 2430 | T -> C |
| 2495 | A -> |
| 2527 | A -> |
| 2737 | A -> G |
| 2989 | A -> G |
| 3029 | G -> A |
| 3241 | T -> C |

Variant protein HSCP2_P16 (SEQ ID NO:316) according to the present invention is encoded by transcript HSCP2_T19 (SEQ ID NO:265) One or more alignments to one or more previously published protein sequences are given in the alignment table on the attached CD-ROM. A brief description of the relationship of the variant protein according to the present invention to each such aligned protein is as follows:

1. Comparison report between HSCP2_P16 (SEQ ID NO:316) and CERU_HUMAN (SEQ ID NO:308):

A. An isolated chimeric polypeptide encoding for HSCP2_P16 (SEQ ID NO:316), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREFVVMFSVVDENFSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKIIVRHYYIAAEEIIWNYAPSGIDIFTKENLTAPGSDSAVFFEQGTTRIGGSY KKLVYREYTDASFTNRKERGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTKDIFTGLIGPMKICKKGSLHANGRQKDVDKEFYLFPTVFDENESLLLEDNIRMFT TAPDQVDKEDEDFQESNKMH corresponding to amino acids 1-621 of CERU_HUMAN (SEQ ID NO:308), which also corresponds to amino acids 1-621 of HSCP2_P16 (SEQ ID NO:316), a second bridging amino acid sequence comprising of W, and a third amino acid sequence being at least 90% homologous to TFNVECLTTDHYTGGMKQKYTVNQCRRQSEDSTFYLGERTYYIAAVEVEWDYSPQREW EKELHHLQEQNVSNAFLDKGEFYIGSKYKKVVYRQYTDSTFRVPVERKAEEEHLGILGP QLHADVGDKVKIIFKNMATRPYSIHAHGVQTESSTVTPTLPGETLTYVWKIPERSGAGTE DSACIPWAYYSTVDQVKDLYSGLIGPLIVCRRPYLKVFNPRRKLEFALLFLVFDENESWY LDDNIKTYSDHPEKVNKDDEEFIESNKMHAINGRMFGNLQGLTMHVGDEVNWYLMGM GNEIDLHTVHFHGHSFQYKHRGVYSSDVFDIFPGTYQTLEMFPRTPGIWLLHCHVTDHIH AGMETTYTVLQNEDTKSG corresponding to amino acids 694-1065 of CERU_HUMAN (SEQ ID NO:308), which also corresponds to amino acids 623-994 of HSCP2_P16 (SEQ ID NO:316), wherein said first amino acid sequence, second amino acid sequence and third amino acid sequence are contiguous and in a sequential order.

B. An isolated polypeptide encoding for an edge portion of HSCP2_P16 (SEQ ID NO:316), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise HWT having a structure as follows (numbering according to HSCP2_P16 (SEQ ID NO:316): a sequence starting from any of amino acid numbers 621-x to 621; and ending at any of amino acid numbers 623+((n−3)−x), in which x varies from 0 to n−3.

3. Comparison report between HSCP2_P16 (SEQ ID NO:316) and Q6NSB2_HUMAN (SEQ ID NO:309):

A. An isolated chimeric polypeptide encoding for HSCP2_P16 (SEQ ID NO:316), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P16 (SEQ ID NO:316), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMH WTFNVECLTTDHYTGGMKQKYTVNQCRRQSEDSTFYLGERTYYIAAVEVEWDYSPQRE WEKELHHLQEQNVSNAFLDKGEFYIGSKYKKVVYRQYTDSTFRVPVERKAEEEHLGILG PQLIIADVGDKVKIIFKNMATRPYSIHAHGVQTESSTVTPTLPGETLTYVWIPERSGAGT EDSACIPWAYYSTVDQVKDLYSGLIGPLIVCRRPYLKVFNPRRKLEFALLFLVFDENESW YLDDNIKTYSDHPEKVNKDDEEFIESNKMHAINGRMFGNLQGLTMHVGDEVNWYLMG MGNEIDLHTVHFHGHSFQYKHRGVYSSDVFDIFPGTYQTLEMFPRTPGIWLLHCHVTDHI HAGMETTYTVLQNEDTKSG (SEQ ID NO:416) corresponding to amino acids 208-994 of HSCP2_P16 (SEQ ID NO:316), wherein said first amino acid sequence and second amino acid sequence are contiguous and in a sequential order.

B. An isolated polypeptide encoding for an edge portion of HSCP2_P16 (SEQ ID NO:316), comprising an amino acid sequence being at least 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMH WTFNVECLTTDHYTGGMKQKYTVNQCRRQSEDSTFYLGERTYYIAAVEVEWDYSPQRE WEKELHHLQEQNVSNAFLDKGEFYIGSKYKKVVYRQYTDSTFRVPVERKAEEEHLGILG PQLHADVGDKVKIIFKNMATRPYSIHAHGVQTESSTVTPTLPGETLTYVWKIPERSGAGT EDSACIPWAYYSTVDQVKDLYSGLIGPLIVCRRPYLKVFNPRRKLEFALLFLVFDENESW YLDDNIKTYSDHPEKVNKDDEEFIESNKMHAINGRMFGNLQGLTMHVGDEVNWYLMG MGNEIDLHTVHFHGHSFQYKHRGVYSSDVFDIFPGTYQTLEMFPRTPGIWLLHCHVTDHI HAGMETTYTVLQNEDTKSG (SEQ ID NO:416) of HSCP2_P16 (SEQ ID NO:316).

The localization of the variant protein was determined according to results from a number of different software programs and analyses, including analyses from SignalP and other specialized programs. The variant protein is believed to be secreted.

Variant protein HSCP2_P16 (SEQ ID NO:316) also has the following non-silent SNPs (Single Nucleotide Polymorphisms) as listed in Table 143, (given according to their position(s) on the amino acid sequence, with the alternative amino acid(s) listed).

TABLE 143
Amino acid mutations

| SNP position(s) | |
|---|---|
| on amino acid | Alternative |
| sequence | amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |
| 656 | F -> S |
| 678 | R -> |
| 688 | Q -> |
| 856 | E -> K |

The glycosylation sites of variant protein HSCP2_P16 (SEQ ID NO:316), as compared to the known protein Ceruloplasmin precursor (SEQ ID NO:308), are described in Table 144 (given according to their position(s) on the amino acid sequence in the first column; the second column indicates whether the glycosylation site is present in the variant protein; and the last column indicates whether the position is different on the variant protein).

TABLE 144
Glycosylation site(s)

| Position(s) on | | |
|---|---|---|
| known amino | Present in | Position(s) on |
| acid sequence | variant protein? | variant protein |
| 138 | Yes | 138 |
| 358 | Yes | 358 |
| 397 | Yes | 397 |
| 691 | Yes | 691 |

Variant protein HSCP2_P16 (SEQ ID NO:316) is encoded by the transcript HSCP2_T19 (SEQ ID NO:265), for which the coding portion starts at position 251 and ends at position 3232. The transcript also has the following SNPs as listed in Table 145 (given according to their position on the nucleotide sequence, with the alternative nucleic acid listed).

TABLE 145
Nucleic acid SNPs

| SNP position(s) | |
|---|---|
| on nucleotide | Alternative |
| sequence | nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |
| 2152 | C -> T |
| 2217 | T -> C |
| 2282 | A -> |
| 2314 | A -> |
| 2524 | A -> G |
| 2776 | A -> G |
| 2816 | G -> A |
| 3028 | T -> C |
| 3064 | A -> G |

Variant protein HSCP2_P18 (SEQ ID NO:317) according to the present invention is encoded by transcript HSCP2_T21 (SEQ ID NO:267) One or more alignments to one or more previously published protein sequences are given in the alignment table on the attached CD-ROM. A brief description of the relationship of the variant protein according to the present invention to each such aligned protein is as follows:

1. Comparison report between HSCP2_P18 (SEQ ID NO:317) and CERU_HUMAN (SEQ ID NO:308):

A. An isolated chimeric polypeptide encoding for HSCP2_P18 (SEQ ID NO:317), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHE corresponding to amino acids 1-131 of CERU_HUMAN (SEQ ID NO:308), which also corresponds to amino acids 1-131 of HSCP2_P18 (SEQ ID NO:317), a second bridging amino acid sequence comprising of A, and a third amino acid sequence being at least 90% homologous to VNGYTFGSLPGLSMCAEDRVKWYLFGMGNEVDVHAAFFHGQALTNKNYRIDTINLFPA TLFDAYMVAQNPGEWMLSCQNLNLKAGLQAFFQVQECNKSSSKDNIRGKHVRHYYIA AEEIIWNYAPSGIDIFTKENLTAPGSDSAVFFEQGTTRIGGSYKKLVYREYTDASFTNRKE RGPEEEHLGILGPVIWAEVGDTIRVTFHNKGAYPLSIEPIGVRFNKNNEGTYYSPNYNPQS RSVPPSASIVAPTETFTYEWTVPKEVGPTNADPVCLAKMYYSAVDPTKDIFTGLIGPMKI CKKGSLHANGRQKDVDKEFYLFPTVFDENESLLLEDNIRMFTTAPDQVDKEDEDFQESN KMHSMNGFMYGNQPGLTMCKGDSVVWYLFSAGNEADVHGIYFSGNTYLWRGERRDT ANLFPQTSLTLHMWPDTEGTFNVECLTTDHYTGGMKQKYTVNQCRRQSEDSTFYLGER TYYIAAVEVEWDYSPQREWEKELHHLQEQNVSNAFLDKGEFYIGSKYKKVVYRQYTDS TFRVPVERKAEEEHLGILGPQLHADVGDKVKIIFKNMATRPYSIHAHGVQTESSTVTPTLP GETLTYVWKIPERSGAGTEDSACIPWAYYSTVDQVKDLYSGLIGPLIVCRRPYLKVFNPR RKLEFALLFLVFDENESWYLDDNIKTYSDHPEKVNKDDEEFIESNKMHAINGRMFGNLQ GLTMHVGDEVNWYLMGMGNEIDLHTVHFHGHSFQYKHRGVYSSDVFDIFPGTYQTLE MFPRTPGIWLLHCHVTDHIHAGMETTYTVLQNEDTKSG corresponding to amino acids 262-1065 of CERU_HUMAN (SEQ ID NO:308), which also corresponds to amino acids 133-936 of HSCP2_P18 (SEQ ID NO:317), wherein said first amino acid sequence, second amino acid sequence and third amino acid sequence are contiguous and in a sequential order.

B. An isolated polypeptide encoding for an edge portion of HSCP2_P18 (SEQ ID NO:317), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least 3 amino acids comprise EAV having a structure as follows (numbering according to HSCP2_P18 (SEQ ID NO:317): a sequence starting from any of amino acid numbers 131-x to 131; and ending at any of amino acid numbers 133+((n−3)−x), in which x varies from 0 to n−3.

3. Comparison report between HSCP2_P18 (SEQ ID NO:317) and Q6NSB2_HUMAN (SEQ ID NO:309):

A. An isolated chimeric polypeptide encoding for HSCP2_P18 (SEQ ID NO:317), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHE corresponding to amino acids 1-131 of Q6NSB2_HUMAN (SEQ ID NO:309), which also corresponds to amino acids 1-131 of HSCP2_P18 (SEQ ID NO:317), and a second amino acid sequence being at least 70%, optionally at least 80%, preferably at least 85%, more preferably at least 90% and most preferably at least 95% homologous to a polypeptide having the sequence AVNGYTFGSLPGLSMCAEDRVKWYLFGMGNEVDVHAAFFHGQALTNKNYRTINLFP ATLFDAYMVAQNPGEWMLSCQNLNHLKAGLQAFFQVQECNKSSSKDNIRGKHVRHYYI AAEEIIWNYAPSGIDIFTKENLTAPGSDSAVFFEQGTTRIGGSYKKLVYREYTDASFTNRK ERGPEEEIHLGILGPVIWAEVGDTIRVTFHNKGAYPLSIEPIGVRFNKNNEGTYYSPNYNPQ SRSVPPSASHVAPTETFTYEWTVPKEVGPTNADPVCLAKMYYSAVDPTKDIFTGLIGPMK ICKKGSLHANGRQKDVDKEFYLFPTVFDENESLLLEDNIRMFTTAPDQVDKEDEDFQESN KMHSMNGFMYGNQPGLTMCKGDSVVWYLFSAGNEADVHGIYFSGNTYLWRGERRDT ANLFPQTSLTLHMWPDTEGTFNVECLTTDHYTGGMKQKYTVNQCRRQSEDSTFYLGER TYYIAAVEVEWDYSPQREWEKELHHLQEQNVSNAFLDKGEFYIGSKYKKVVYRQYTDS TFRVPVERKAEEEHLGILGPQLHADVGDKVKnFKNMATRPYSIHAHGVQTESSTVTPTLP GETLTYVWKIPERSGAGTEDSACIPWAYYSTVDQVKDLYSGLIGPLIVCRRPYLKVFNPR RKLEFALLFLVFDENESWYLDDNIKTYSDHPEKVNKDDEEFIESNKMHAINGRMFGNLQ GLTMHVGDEVNWYLMGMGNEIDLHTVHFHGHSFQYKHRGVYSSDVFDIFPGTYQTLE MFPRTPGIWLLHCHVTDHIHAGMETTYTVLQNEDTKSG (SEQ ID NO:417) corresponding to amino acids 132-936 of HSCP2_P18 (SEQ ID NO:317), wherein said first amino acid sequence and second amino acid sequence are contiguous and in a sequential order.

B. An isolated polypeptide encoding for an edge portion of HSCP2_P18 (SEQ ID NO:317), comprising an amino acid sequence being at least 70%, optionally at least about 80%, preferably at least about 85%, more preferably at least about 90% and most preferably at least about 95% homologous to the sequence AVNGYTFGSLPGLSMCAEDRVKWYLFGMGNEVDVHAAFFHGQALTNKNYRIDTINLFP ATLFDAYMVAQNPGEWMLSCQNLNHLKAGLQAFFQVQECNKSSSKDNIRGKHVRHYYI AAEEIIWNYAPSGIDIFTKENLTAPGSDSAVFFEQGTTRIGGSYKKLVYREYTDASFTNRK ERGPEEEHLGILGPVIWAEVGDTIRVTFHNKGAYPLSIEPIGVRFNKNNEGTYYSPNYNPQ SRSVPPSASHVAPTETFTYEWTVPKEVGPTNADPVCLAKMYYSAVDPTKDIFTGLIGPMK ICKKGSLHANGRQKDVDKEFYLFPTVFDENESLLLEDMRMFTTAPDQVDKEDEDFQESN KMHSMNGFMYGNQPGLTMCKGDSVVWYLFSAGNEADVHGIYFSGNTYLWRGERRDT ANLFPQTSLTLHMWPDTEGTFNVECLTTDHYTGGMKQKYTVNQCRRQSEDSTFYLGER TYYIAAVEVEWDYSPQREWEKELHHLQEQNVSNAFLDKGEFYIGSKYKKVVYRQYTDS TFRVPVERKAEEEHLGILGPQLHADVGDKVKIIFKNMATRPYSIHAHGVQTESSTVTPTLP GETLTYVWKIPERSGAGTEDSACIPWAYYSTVDQVKDLYSGLIGPLIVCRRPYLKVFNPR RKEFALLFLVFDENESWYLDDNIKTYSDHPEKVNKDDEEFIESNKMHAINGRMFGNLQ GLTMHVGDEVNWYLMGMGNEIDLHTVHFHGHSFQYKHRGVYSSDVFDIFPGTYQTLE MFPRTPGIWLLHCHVTDHIHAGMETTYTVLQNEDTKSG (SEQ ID NO:417) of HSCP2_P18 (SEQ ID NO:317).

The localization of the variant protein was determined according to results from a number of different software programs and analyses, including analyses from SignalP and other specialized programs. The variant protein is believed to be secreted.

Variant protein HSCP2_P18 (SEQ ID NO:317) also has the following non-silent SNPs (Single Nucleotide Polymorphisms) as listed in Table 146, (given according to their position(s) on the amino acid sequence, with the alternative amino acid(s) listed; the last column indicates whether the SNP is known or not; the presence of known SNPs in variant protein HSCP2_P18 (SEQ ID NO:317) sequence provides support for the deduced sequence of this variant protein according to the present invention).

TABLE 146
Amino acid mutations

| SNP position(s) | |
|---|---|
| on amino acid | Alternative |
| sequence | amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 146 | M -> T |
| 157 | F -> L |
| 169 | F -> S |
| 176 | T -> A |
| 316 | H -> Y |
| 322 | P -> A |
| 348 | P -> L |
| 365 | N -> |
| 378 | S -> P |
| 385 | T -> |
| 385 | T -> A |
| 406 | L -> P |
| 415 | D -> E |
| 455 | V -> A |
| 469 | R -> K |
| 478 | V -> G |
| 531 | F -> S |
| 547 | N -> |
| 598 | F -> S |
| 620 | R -> |
| 630 | Q -> |
| 798 | E -> K |

The glycosylation sites of variant protein HSCP2_P18 (SEQ ID NO:317), as compared to the known protein Ceruloplasmin precursor (SEQ ID NO:308), are described in Table 147 (given according to their position(s) on the amino acid sequence in the first column; the second column indicates whether the glycosylation site is present in the variant protein; and the last column indicates whether the position is different on the variant protein).

TABLE 147
Glycosylation site(s)

| Position(s) on | | |
|---|---|---|
| known amino | Present in | Position(s) on |
| acid sequence | variant protein? | variant protein |
| 138 | No | |
| 229 | Yes | 229 |
| 268 | Yes | 268 |
| 633 | Yes | 633 |

Variant protein HSCP2_P18 (SEQ ID NO:317) is encoded by the transcript HSCP2_T21 (SEQ ID NO:267), for which the coding portion starts at position 251 and ends at position 3058. The transcript also has the following SNPs as listed in Table 148 (given according to their position on the nucleotide sequence, with the alternative nucleic acid listed).

TABLE 148
Nucleic acid SNPs

| SNP position(s) on nucleotide | |
|---|---|
| sequence | Alternative nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 667 | A -> G |
| 687 | T -> C |
| 721 | T -> G |
| 756 | T -> C |
| 776 | A -> G |
| 898 | A -> G |
| 901 | C -> T |
| 1196 | C -> T |
| 1214 | C -> G |
| 1231 | G -> A |
| 1293 | C -> T |
| 1344 | A -> |
| 1382 | T -> C |
| 1403 | A -> G |
| 1403 | A -> |
| 1465 | T -> C |
| 1467 | T -> C |
| 1495 | T -> A |
| 1614 | T -> C |
| 1656 | G -> A |
| 1669 | T -> C |
| 1683 | T -> G |
| 1753 | T -> C |
| 1813 | A -> C |
| 1842 | T -> C |
| 1890 | A -> |
| 1978 | C -> T |
| 2043 | T -> C |
| 2108 | A -> |
| 2140 | A -> |
| 2350 | A -> G |
| 2602 | A -> G |
| 2642 | G -> A |
| 2854 | T -> C |
| 2890 | A -> G |

Variant protein HSCP2_P21 (SEQ ID NO:318) according to the present invention is encoded by transcript HSCP2_T25 (SEQ ID NO:269) One or more alignments to one or more previously published protein sequences are given in the alignment table on the attached CD-ROM. A brief description of the relationship of the variant protein according to the present invention to each such aligned protein is as follows:

1. Comparison report between HSCP2_P21 (SEQ ID NO:318) and CERU_HUMAN (SEQ ID NO:308):

A. An isolated chimeric polypeptide encoding for HSCP2_P21 (SEQ ID NO:318), comprising a first amino acid sequence being at least 90% homologous to MKILILGIFLFLCSTPAWAKEKHYYIGHETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREFVVMFSWDENFSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKHVRHYYIAAEEIIWNYAPSGIDIFTKENLTAPGSDSAVFFEQGTTRIGGSY KKLVYREYTDASFTNRKERGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTKDIFTGLIGPMKICKKGSLHANGRQKDVDKEFYLFPTVFDENESLLLEDNIRMFT TAPDQVDKEDEDFQESNKMHSMNGFMYGNQPGLTMCKGDSVVWYLFSAGNEADVHGI YFSGNTYLWRGERRDTANLFPQTSLTLHMWPDTEGTFNVECLTTDHYTGGMKQKYTVN QCRRQSEDSTFYLGERTYYIAAVEVEWDYSPQREWEKELHHLQEQNVSNAFLDKGEFYI GSKYKKVVYRQYTDSTFRVPVERKAEEEHLGILGPQLHADVGDKVKIIFKNMATRPYSIH AHGVQTESSTVTPTLPG corresponding to amino acids 1-852 of CERU_HUMAN (SEQ ID NO:308), which also corresponds to amino acids 1-852 of HSCP2_P21 (SEQ ID NO:318), and a second amino acid sequence being at least 70%, optionally at least 80%, preferably at least 85%, more preferably at least 90% and most preferably at least 95% homologous to a polypeptide having the sequence THGGGGGGGAF (SEQ ID NO:418) corresponding to amino acids 853-863 of HSCP2_P21 (SEQ ID NO:318), wherein said first amino acid sequence and second amino acid sequence are contiguous and in a sequential order.

B. An isolated polypeptide encoding for an edge portion of HSCP2_P21 (SEQ ID NO:318), comprising an amino acid sequence being at least 70%, optionally at least about 80%, preferably at least about 85%, more preferably at least about 90% and most preferably at least about 95% homologous to the sequence THGGGGGGGAF (SEQ ID NO:418) of HSCP2_P21 (SEQ ID NO:318).

3. Comparison report between HSCP2_P21 (SEQ ID NO:318) and Q6NSB2_HUMAN (SEQ ID NO:309):

A. An isolated chimeric polypeptide encoding for HSCP2_P21 (SEQ ID NO:318), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P21 (SEQ ID NO:318), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGTHG GGGGGGAF (SEQ ID NO:419) corresponding to amino acids 208-863 of HSCP2_P21 (SEQ ID NO:318), wherein said first amino acid sequence and second amino acid sequence are contiguous and in a sequential order.

B. An isolated polypeptide encoding for an edge portion of HSCP2_P21 (SEQ ID NO:318), comprising an amino acid sequence being at least 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNIHLKAGLQAFFQVQECNKSSSKDNIRGKHVRHYYIAAEEII WNYAPSGIDIFTKENLTAPGSDSAVFFEQGTTRIGGSYKKLVYREYTDASFNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHS MNGFMYGNQPGLTMCKGDSVVWYLFSAGNEADVHGIYFSGNTYLWRGERRDTANLFP QTSLTLHMWPDTEGTFNVECLTTDHYTGGMKQKYTVNQCRRQSEDSTFYLGERTYYIA AVEVEWDYSPQREWEKELHHLQEQNVSNAFLDKGEFYIGSKYKKVVYRQYTDSTFRVP VERKAEEEHLGILGPQLHADVGDKVKIIFKNMATRPYSIHAHGVQTESSTVTPTLPGTHG GGGGGGAF (SEQ ID NO:419) of HSCP2_P21 (SEQ ID NO:318).

The localization of the variant protein was determined according to results from a number of different software programs and analyses, including analyses from SignalP and other specialized programs. The variant protein is believed to be secreted.

Variant protein HSCP2_P21 (SEQ ID NO:318) also has the following non-silent SNPs (Single Nucleotide Polymorphisms) as listed in Table 149, (given according to their position(s) on the amino acid sequence, with the alternative amino acid(s) listed).

TABLE 149
Amino acid mutations

| SNP position(s) on amino acid | |
|---|---|
| sequence | Alternative amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |
| 660 | F -> S |
| 676 | N -> |
| 727 | F -> S |
| 749 | R -> |
| 759 | Q -> |

The glycosylation sites of variant protein HSCP2_P21 (SEQ ID NO:318), as compared to the known protein Ceruloplasmin precursor (SEQ ID NO:308), are described in Table 150 (given according to their position(s) on the amino acid sequence in the first column; the second column indicates whether the glycosylation site is present in the variant protein; and the last column indicates whether the position is different on the variant protein).

TABLE 150
Glycosylation site(s)

| Position(s) on known | Present | Position(s) |
|---|---|---|
| amino acid sequence | in variant protein? | on variant protein |
| 138 | Yes | 138 |
| 358 | Yes | 358 |
| 397 | Yes | 397 |
| 762 | Yes | 762 |

Variant protein HSCP2_P21 (SEQ ID NO:318) is encoded by the transcript HSCP2_T25 (SEQ ID NO:269), for which the coding portion starts at position 251 and ends at position 2839. The transcript also has the following SNPs as listed in Table 151 (given according to their position on the nucleotide sequence, with the alternative nucleic acid listed).

TABLE 151
Nucleic acid SNPs

| SNP position(s) on nucleotide | |
|---|---|
| sequence | Alternative nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |
| 2140 | T -> C |
| 2200 | A -> C |
| 2229 | T -> C |
| 2277 | A -> |
| 2365 | C -> T |
| 2430 | T -> C |
| 2495 | A -> |
| 2527 | A -> |
| 2737 | A -> G |
| 2900 | G -> A |
| 3134 | T -> A |
| 3207 | A -> C |
| 3421 | G -> A |
| 3481 | A -> G |

Variant protein HSCP2_P23 (SEQ ID NO:319) according to the present invention is encoded by transcript HSCP2_T28 (SEQ ID NO:270) One or more alignments to one or more previously published protein sequences are given in the alignment table on the attached CD-ROM. A brief description of the relationship of the variant protein according to the present invention to each such aligned protein is as follows:

1. Comparison report between HSCP2_P23 (SEQ ID NO:319) and CERU_HUMAN (SEQ ID NO:308):

A. An isolated chimeric polypeptide encoding for HSCP2_P23 (SEQ ID NO:319), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREFVVMFSVVDENSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKHVRHYYIAAEEIIWNYAPSGIDIFTKENLTAPGSDSAVFFEQGTTRIGGSY KKLVYREYTDASFTNRKERGPEEEHLGILGPVIWAEVGDTIRVTFHNKGAYPLSIEPIGVR FNKNNEGTYYSPNYNPQSRSVPPSASHVAPTETFTYEWTVPKEVGPTNADPVCLAKMYY SAVDPTKDIFTGLIGPMKICKKGSLHANGRQKDVDKEFYLFPTVFDENESLLLEDNIRMFT TAPDQVDKEDEDFQESNKMH corresponding to amino acids 1-621 of CERU_HUMAN (SEQ ID NO:308), which also corresponds to amino acids 1-621 of HSCP2_P23 (SEQ ID NO:319), and a second amino acid sequence being at least 70%, optionally at least 80%, preferably at least 85%, more preferably at least 90% and most preferably at least 95% homologous to a polypeptide having the sequence CKYCIIHQSTKLF (SEQ ID NO:420) corresponding to amino acids 622-634 of HSCP2_P23 (SEQ ID NO:319), wherein said first amino acid sequence and second amino acid sequence are contiguous and in a sequential order.

B. An isolated polypeptide encoding for an edge portion of HSCP2_P23 (SEQ ID NO:319), comprising an amino acid sequence being at least 70%, optionally at least about 80%, preferably at least about 85%, more preferably at least about 90% and most preferably at least about 95% homologous to the sequence CKYCIIHQSTKLF (SEQ ID NO:420) of HSCP2_P23 (SEQ ID NO:319).

3. Comparison report between HSCP2_P23 (SEQ ID NO:319) and Q6NSB2_HUMAN (SEQ ID NO:309):

A. An isolated chimeric polypeptide encoding for HSCP2_P23 (SEQ ID NO:319), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P23 (SEQ ID NO:319), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTINLFPATLFDA YMVAQNPGEWMLSCQNLNIHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHC KYCIIHQSTKLF (SEQ ID NO:421) corresponding to amino acids 208-634 of HSCP2_P23 (SEQ ID NO:319), wherein said first amino acid sequence and second amino acid sequence are contiguous and in a sequential order.

B. An isolated polypeptide encoding for an edge portion of HSCP2_P23 (SEQ ID NO:319), comprising an amino acid sequence being at least 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSVP PSASHVAPTETFTYEWTVPKEVGPTNADPVCLAKMYYSAVDPTKDIFTGLIGPMKICKKG SLHANGRQKDVDKEFYLFPTVFDENESLLLEDNIRMFTTAPDQVDKEDEDFQESNKMHC KYCIIHQSTKLF (SEQ ID NO:421) of HSCP2_P23 (SEQ ID NO:319).

The localization of the variant protein was determined according to results from a number of different software programs and analyses, including analyses from SignalP and other specialized programs. The variant protein is believed to secreted.

Variant protein HSCP2_P23 (SEQ ID NO:319) also has the following non-silent SNPs (Single Nucleotide Polymorphisms) as listed in Table 152, (given according to their position(s) on the amino acid sequence, with the alternative amino acid(s) listed; the last column indicates whether the SNP is known or not; the presence of known SNPs in variant protein HSCP2_P23 (SEQ ID NO:319) sequence provides support for the deduced sequence of this variant protein according to the present invention).

TABLE 152
Amino acid mutations

| SNP position(s) on amino acid | |
|---|---|
| sequence | Alternative amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |
| 507 | S -> P |
| 514 | T -> |
| 514 | T -> A |
| 535 | L -> P |
| 544 | D -> E |
| 584 | V -> A |
| 598 | R -> K |
| 607 | V -> G |

The glycosylation sites of variant protein HSCP2_P23 (SEQ ID NO:319), as compared to the known protein Ceruloplasmin precursor (SEQ ID NO:308), are described in Table 153 (given according to their position(s) on the amino acid sequence in the first column; the second column indicates whether the glycosylation site is present in the variant protein; and the last column indicates whether the position is different on the variant protein).

TABLE 153
Glycosylation site(s)

| Position(s) on known | Present | Position(s) |
|---|---|---|
| amino acid sequence | in variant protein? | on variant protein |
| 138 | Yes | 138 |
| 358 | Yes | 358 |
| 397 | Yes | 397 |
| 762 | No | |

Variant protein HSCP2_P23 (SEQ ID NO:319) is encoded by the transcript HSCP2_T28 (SEQ ID NO:270), for which the coding portion starts at position 251 and ends at position 2152. The transcript also has the following SNPs as listed in Table 154 (given according to their position on the nucleotide sequence, with the alternative nucleic acid listed).

TABLE 154
Nucleic acid SNPs

| SNP position(s) on nucleotide | |
|---|---|
| sequence | Alternative nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |
| 1769 | T -> C |
| 1790 | A -> G |
| 1790 | A -> |
| 1852 | T -> C |
| 1854 | T -> C |
| 1882 | T -> A |
| 2001 | T -> C |
| 2043 | G -> A |
| 2056 | T -> C |
| 2070 | T -> G |

Variant protein HSCP2_P24 (SEQ ID NO:320) according to the present invention is encoded by transcript HSCP2_T29 (SEQ ID NO:271) One or more alignments to one or more previously published protein sequences are given in the alignment table on the attached CD-ROM. A brief description of the relationship of the variant protein according to the present invention to each such aligned protein is as follows:

1. Comparison report between HSCP2_P24 (SEQ ID NO:320) and CERU_HUMAN (SEQ ID NO:308):

A. An isolated chimeric polypeptide encoding for HSCP2_P24 (SEQ ID NO:320), comprising a first amino acid sequence being at least 90% homologous to MKILILGIFLFLCSTPAWAKEKHYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDKEKEKHIDREVVMFSVVDENFSWYLEDNIKTYCSEP EKVDKDNEDFQESNRMYSVNGYTFGSLPGLSMCAEDRVKWYLFGMGNEVDVHAAFFH GQALTNKNYRIDTINLFPATLFDAYMVAQNPGEWMLSCQNLNHLKAGLQAFFQVQECN KSSSKDNIRGKHVRHIYYIAAEEIIWNYAPSGIDIFTKENLTAPGSDSAVFFEQGTTRIGGSY KKLVYREYTDASFTNRKERGPEEEIHLGILGPVIWAEVGDTIRVTFHNKGAYPLSIEPIGVR FNKNNEGTYYSPNYNPQSRS corresponding to amino acids 1-501 of CERU_HUMAN (SEQ ID NO:308), which also corresponds to amino acids 1-501 of HSCP2_P24 (SEQ ID NO:320), and a second amino acid sequence being at least 70%, optionally at least 80%, preferably at least 85%, more preferably at least 90% and most preferably at least 95% homologous to a polypeptide having the sequence EYTALCNK (SEQ ID NO:422) corresponding to amino acids 502-509 of HSCP2_P24 (SEQ ID NO:320), wherein said first amino acid sequence and second amino acid sequence are contiguous and in a sequential order.

B. An isolated polypeptide encoding for an edge portion of HSCP2_P24 (SEQ ID NO:320), comprising an amino acid sequence being at least 70%, optionally at least about 80%, preferably at least about 85%, more preferably at least about 90% and most preferably at least about 95% homologous to the sequence EYTALCNK (SEQ ID NO:422) of HSCP2_P24 (SEQ ID NO:320).

3. Comparison report between HSCP2_P24 (SEQ ID NO:320) and Q6NSB2_HUMAN (SEQ ID NO:309):

A. An isolated chimeric polypeptide encoding for HSCP2_P24 (SEQ ID NO:320), comprising a first amino acid sequence being at least 90% homologous to MKILILGIFLFLCSTPAWAKEKYYIGIIETTWDYASDHGEKKLISVDTEHSNIYLQNGPDR IGRLYKKALYLQYTDETFRTTIEKPVWLGFLGPIIKAETGDKVYVHLKNLASRPYTFHSH GITYYKEHEGAIYPDNTTDFQRADDKVYPGEQYTYMLLATEEQSPGEGDGNCVTRIYHS HIDAPKDIASGLIGPLIICKKDSLDK corresponding to amino acids 1-207 of Q6NSB2_HUMAN (SEQ ID NO:309), which also corresponds to amino acids 1-207 of HSCP2_P24 (SEQ ID NO:320), and a second amino acid sequence being at least 70%, optionally at least 80%, preferably at least 85%, more preferably at least 90% and most preferably at least 95% homologous to a polypeptide having the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSEY TALCNK (SEQ ID NO:423) corresponding to amino acids 208-509 of HSCP2_P24 (SEQ ID NO:320), wherein said first amino acid sequence and second amino acid sequence are contiguous and in a sequential order.

B. An isolated polypeptide encoding for an edge portion of HSCP2_P24 (SEQ ID NO:320), comprising an amino acid sequence being at least 70%, optionally at least about 80%, preferably at least about 85%, more preferably at least about 90% and most preferably at least about 95% homologous to the sequence EKEKHIDREFVVMFSVVDENFSWYLEDNIKTYCSEPEKVDKDNEDFQESNRMYSVNGYT FGSLPGLSMCAEDRVKWYLFGMGNEVDVHAAFFHGQALTNKNYRIDTINLFPATLFDA YMVAQNPGEWMLSCQNLNHLKAGLQAFFQVQECNKSSSKDNIRGKHVRHYYIAAEEII WNYAPSGIDIFTKENLTAPGSDSAVFFEQGTTRIGGSYKKLVYREYTDASFTNRKERGPE EEHLGILGPVIWAEVGDTIRVTFHNKGAYPLSIEPIGVRFNKNNEGTYYSPNYNPQSRSEY TALCNK (SEQ ID NO:423) of HSCP2_P24 (SEQ ID NO:320).

The localization of the variant protein was determined according to results from a number of different software programs and analyses, including analyses from SignalP and other specialized programs. The variant protein is believed to be secreted.

Variant protein HSCP2_P24 (SEQ ID NO:320) also has the following non-silent SNPs (Single Nucleotide Polymorphisms) as listed in Table 155, (given according to their position(s) on the amino acid sequence, with the alternative amino acid(s) listed).

TABLE 155
Amino acid mutations

| SNP position(s) on amino acid | |
|---|---|
| sequence | Alternative amino acid(s) |
| 26 | I -> |
| 29 | I -> |
| 37 | S -> P |
| 47 | V -> |
| 53 | N -> S |
| 54 | I -> V |
| 63 | I -> |
| 92 | F -> S |
| 117 | Y -> N |
| 148 | K -> R |
| 173 | N -> |
| 190 | A -> |
| 190 | A -> G |
| 235 | N -> D |
| 253 | F -> L |
| 275 | M -> T |
| 286 | F -> L |
| 298 | F -> S |
| 305 | T -> A |
| 445 | H -> Y |
| 451 | P -> A |
| 477 | P -> L |
| 494 | N -> |

The glycosylation sites of variant protein HSCP2_P24 (SEQ ID NO:320), as compared to the known protein Ceruloplasmin precursor (SEQ ID NO:308), are described in Table 156 (given according to their position(s) on the amino acid sequence in the first column; the second column indicates whether the glycosylation site is present in the variant protein; and the last column indicates whether the position is different on the variant protein).

TABLE 156
Glycosylation site(s)

| Position(s) on known | Present | Position(s) |
|---|---|---|
| amino acid sequence | in variant protein? | on variant protein |
| 138 | Yes | 138 |
| 358 | Yes | 358 |
| 397 | Yes | 397 |
| 762 | No | |

Variant protein HSCP2_P24 (SEQ ID NO:320) is encoded by the transcript(HSCP2_T29 (SEQ ID NO:271), for which the coding portion starts at position 251 and ends at position 1777. The transcript also has the following SNPs as listed in Table 157 (given according to their position on the nucleotide sequence, with the alternative nucleic acid listed).

TABLE 157
Nucleic acid SNPs

| SNP position(s) on nucleotide | |
|---|---|
| sequence | Alternative nucleic acid(s) |
| 327 | T -> |
| 336 | T -> |
| 359 | T -> C |
| 361 | T -> C |
| 390 | T -> |
| 408 | A -> G |
| 410 | A -> G |
| 438 | T -> |
| 525 | T -> C |
| 592 | T -> C |
| 599 | T -> A |
| 693 | A -> G |
| 769 | T -> |
| 808 | A -> G |
| 819 | C -> G |
| 819 | C -> |
| 838 | T -> C |
| 953 | A -> G |
| 1007 | T -> C |
| 1054 | A -> G |
| 1074 | T -> C |
| 1108 | T -> G |
| 1143 | T -> C |
| 1163 | A -> G |
| 1285 | A -> G |
| 1288 | C -> T |
| 1583 | C -> T |
| 1601 | C -> G |
| 1618 | G -> A |
| 1680 | C -> T |
| 1731 | A -> |

Variant protein HSCP2_P37 (SEQ ID NO:321) according to the present invention is encoded by transcript HSCP2_T20 (SEQ ID NO:266) One or more alignments to one or more previously published protein sequences are given in the alignment table on the attached CD-ROM. A brief description of the relationship of the variant protein according to the present invention to each such aligned protein is as follows:

1. Comparison report between HSCP2_P37 (SEQ ID NO:321) and CERU_HUMAN (SEQ ID NO:308):

A. An isolated chimeric polypeptide encoding for HSCP2_P37 (SEQ ID NO:321), comprising a first amino acid sequence being at least 90% homologous to MKILILGIFLFLCSTPAWAKEKHYYIGIIETTWDYASDHGEKKLISVDT corresponding to amino acids 1-49 of CERU_HUMAN (SEQ ID NO:308), which also corresponds to amino acids 1-49 of HSCP2_P37 (SEQ ID NO:321), and a second amino acid sequence being at least 70%, optionally at least 80%, preferably at least 85%, more preferably at least 90% and most preferably at least 95% homologous to a polypeptide having the sequence GHLP (SEQ ID NO:424) corresponding to amino acids 50-53 of HSCP2_P37 (SEQ ID NO:321), wherein said first amino acid sequence and second amino acid sequence are contiguous and in a sequential order.

B. An isolated polypeptide encoding for an edge portion of HSCP2_P37 (SEQ ID NO:321), comprising an amino acid sequence being at least 70%, optionally at least about 80%, preferably at least about 85%, more preferably at least about 90% and most preferably at least about 95% homologous to the sequence GHLP (SEQ ID NO:424) of HSCP2_P37 (SEQ ID NO:321).

):),),), SEQ ID NO: ),), SEQ ID NO: ).The localization of the variant protein was determined according to results from a number of different software programs and analyses, including analyses from SignalP and other specialized programs. The variant protein is believed to be secreted.

Variant protein HSCP2_P37 (SEQ ID NO:321) also has the following non-silent SNPs (Single Nucleotide Polymorphisms) as listed in Table 158, (given according to their position(s) on the amino acid sequence, with the alternative amino acid(s) listed).

TABLE 158
Amino acid mutations

| SNP position(s) on amino acid | |
|---|---|
| sequence | Alternative amino acid(s) |
| 26 | I -> |
| 29 | I -> |
| 37 | S -> P |
| 47 | V -> |

The glycosylation sites of variant protein HSCP2_P37 (SEQ ID NO:321), as compared to the known protein Ceruloplasmin precursor (SEQ ID NO:308), are described in Table 159 (given according to their position(s) on the amino acid sequence in the first column; the second column indicates whether the glycosylation site is present in the variant protein; and the last column indicates whether the position is different on the variant protein).

TABLE 159
Glycosylation site(s)

| Position(s) on known amino | |
|---|---|
| acid sequence | Present in variant protein? |
| 138 | No |
| 358 | No |
| 397 | No |
| 762 | No |

Variant protein HSCP2_P37 (SEQ ID NO:321) is encoded by the transcript HSCP2_T20 (SEQ ID NO:266), for which the coding portion starts at position 251 and ends at position 409. The transcript also has the following SNPs as listed in Table 160 (given according to their position on the nucleotide sequence, with the alternative nucleic acid listed).

TABLE 160
Nucleic acid SNPs

| SNP position(s) on nucleotide | |
|---|---|
| sequence | Alternative nucleic acid(s) |
| 327 | T -> |
| 336 | T -> |
| 359 | T -> C |
| 361 | T -> C |
| 390 | T -> |
| 445 | A -> G |
| 521 | T -> |
| 560 | A -> G |
| 571 | C -> G |
| 571 | C -> |
| 590 | T -> C |
| 705 | A -> G |
| 759 | T -> C |
| 806 | A -> G |
| 826 | T -> C |
| 860 | T -> G |
| 895 | T -> C |
| 915 | A -> G |
| 1037 | A -> G |
| 1040 | C -> T |
| 1335 | C -> T |
| 1353 | C -> G |
| 1370 | G -> A |
| 1432 | C -> T |
| 1483 | A -> |
| 1521 | T -> C |
| 1542 | A -> G |
| 1542 | A -> |
| 1604 | T -> C |
| 1606 | T -> C |
| 1634 | T -> A |
| 1753 | T -> C |
| 1795 | G -> A |
| 1808 | T -> C |
| 1822 | T -> G |
| 1892 | T -> C |
| 1952 | A -> C |
| 1981 | T -> C |
| 2029 | A -> |
| 2117 | C -> T |
| 2182 | T -> C |
| 2247 | A -> |
| 2279 | A -> |
| 2489 | A -> G |
| 2741 | A -> G |
| 2781 | G -> A |
| 2993 | T -> C |
| 3029 | A -> G |

Variant protein HSCP2_P39 (SEQ ID NO:322) according to the present invention is encoded by transcript HSCP2_T23 (SEQ ID NO:268) One or more alignments to one or more previously published protein sequences are given in the alignment table on the attached CD-ROM. A brief description of the relationship of the variant protein according to the present invention to each such aligned protein is as follows:

1. Comparison report between HSCP2_P39 (SEQ ID NO:322) and CERU_HUMAN (SEQ ID NO:308):

A. An isolated chimeric polypeptide encoding for HSCP2_P39 (SEQ ID NO:322), comprising a first amino acid sequence being at least 90% homologous to MKILILGIFLFLCSTPAWAKEKHYYIGIIETTWDYASDHGEKKLISVDT corresponding to amino acids 1-49 of CERU_HUMAN (SEQ ID NO:308), which also corresponds to amino acids 1-49 of HSCP2_P39 (SEQ ID NO:322), and a second amino acid sequence being at least 70%, optionally at least 80%, preferably at least 85%, more preferably at least 90% and most preferably at least 95% homologous to a polypeptide having the sequence CEWIHFWKSPRTLHVC (SEQ ID NO:425) corresponding to amino acids 50-65 of HSCP2_P39 (SEQ ID NO:322), wherein said first amino acid sequence and second amino acid sequence are contiguous and in a sequential order.

B. An isolated polypeptide encoding for an edge portion of HSCP2_P39 (SEQ ID NO:322), comprising an amino acid sequence being at least 70%, optionally at least about 80%, preferably at least about 85%, more preferably at least about 90% and most preferably at least about 95% homologous to the sequence CEWIHFWKSPRTLHVC (SEQ ID NO:425) of HSCP2_P39 (SEQ ID NO:322).

2. Comparison report between HSCP2_P39 (SEQ ID NO:322) and Q6NSB2_HUMAN (SEQ ID NO:309):

A. An isolated chimeric polypeptide encoding for HSCP2_P39 (SEQ ID NO:322), comprising a first amino acid sequence being at least 90% homologous to MKILILGIFLFLCSTPAWAKEKHYYIGIIETTWDYASDHGEKKLISVDT corresponding to amino acids 1-49 of Q6NSB2_HUMAN (SEQ ID NO:309), which also corresponds to amino acids 1-49 of HSCP2_P39 (SEQ ID NO:322), and a second amino acid sequence being at least 70%, optionally at least 80%, preferably at least 85%, more preferably at least 90% and most preferably at least 95% homologous to a polypeptide having the sequence CEWIHFWKSPRTLHVC (SEQ ID NO:425) corresponding to amino acids 50-65 of HSCP2_P39 (SEQ ID NO:322), wherein said first amino acid sequence and second amino acid sequence are contiguous and in a sequential order.

B. An isolated polypeptide encoding for an edge portion of HSCP2_P39 (SEQ ID NO:322), comprising an amino acid sequence being at least 70%, optionally at least about 80%, preferably at least about 85%, more preferably at least about 90% and most preferably at least about 95% homologous to the sequence CEWIHFWKSPRTLHVC (SEQ ID NO:425) of HSCP2_P39 (SEQ ID NO:322).

The localization of the variant protein was determined according to results from a number of different software programs and analyses, including analyses from SignalP and other specialized programs. The variant protein is believed to be secreted.

Variant protein HSCP2_P39 (SEQ ID NO:322) also has the following non-silent SNPs (Single Nucleotide Polymorphisms) as listed in Table 161, (given according to their position(s) on the amino acid sequence, with the alternative amino acid(s) listed).

TABLE 161
Amino acid mutations

| SNP position(s) on amino acid | |
|---|---|
| sequence | Alternative amino acid(s) |
| 26 | I -> |
| 29 | I -> |
| 37 | S -> P |
| 47 | V -> |
| 57 | K -> E |

The glycosylation sites of variant protein HSCP2_P39 (SEQ ID NO:322), as compared to the known protein Ceruloplasmin precursor (SEQ ID NO:308), are described in Table 162 (given according to their position(s) on the amino acid sequence in the first column; the second column indicates whether the glycosylation site is present in the variant protein; and the last column indicates whether the position is different on the variant protein).

TABLE 162
Glycosylation site(s)

| Position(s) on known amino | |
|---|---|
| acid sequence | Present in variant protein? |
| 138 | No |
| 358 | No |
| 397 | No |
| 762 | No |

Variant protein HSCP2_P39 (SEQ ID NO:322) is encoded by the transcript HSCP2_T23 (SEQ ID NO:268), for which the coding portion starts at position 251 and ends at position 445. The transcript also has the following SNPs as listed in Table 163 (given according to their position on the nucleotide sequence, with the alternative nucleic acid listed).

TABLE 163
Nucleic acid SNPs

| SNP position(s) | |
|---|---|
| on nucleotide | Alternative |
| sequence | nucleic acid(s) |
| 327 | T -> |
| 336 | T -> |
| 359 | T -> C |
| 361 | T -> C |
| 390 | T -> |
| 419 | A -> G |
| 439 | T -> C |
| 473 | T -> G |
| 508 | T -> C |
| 528 | A -> G |
| 650 | A -> G |
| 653 | C -> T |
| 948 | C -> T |
| 966 | C -> G |
| 983 | G -> A |
| 1045 | C -> T |
| 1096 | A -> |
| 1134 | T -> C |
| 1155 | A -> G |
| 1155 | A -> |
| 1217 | T -> C |
| 1219 | T -> C |
| 1247 | T -> A |
| 1366 | T -> C |
| 1408 | G -> A |
| 1421 | T -> C |
| 1435 | T -> G |
| 1505 | T -> C |
| 1565 | A -> C |
| 1594 | T -> C |
| 1642 | A -> |
| 1730 | C -> T |
| 1795 | T -> C |
| 1860 | A -> |
| 1892 | A -> |
| 2102 | A -> G |
| 2354 | A -> G |
| 2394 | G -> A |
| 2606 | T -> C |
| 2642 | A -> G |

As noted above, cluster HSCP2 features 36 segments, which were listed in Table 118. These segments are portions of nucleic acid sequences which are described herein separately because they are of particular interest. A description of some of these segment according to the present invention is now provided.

Segment cluster HSCP2_N0 (SEQ ID NO:272) according to the present invention is supported by 61 libraries. The number of libraries was determined as previously described. This segment can be found in the following transcript(s): HSCP2_T11 (SEQ ID NO:263), HSCP2_T18 (SEQ ID NO:264), HSCP2_T19 (SEQ ID NO:265), HSCP2_T20 (SEQ ID NO:266), HSCP2_T21 (SEQ ID NO:267), HSCP2_T23 (SEQ ID NO:268), HSCP2_T25 (SEQ ID NO:269), HSCP2_T28 (SEQ ID NO:270), HSCP2_T29 (SEQ ID NO:271), HSCP2_T5 (SEQ ID NO:259), HSCP2_T6 (SEQ ID NO:260), HSCP2_T7 (SEQ ID NO:261) and HSCP2_T9 (SEQ ID NO:262). Table 164 below describes the starting and ending position of this segment on each transcript.

TABLE 164
Segment location on transcripts

| | Segment | Segment |
|---|---|---|
| | starting | ending |
| Transcript name | position | position |
| HSCP2_T11 (SEQ ID NO: 263) | 1 | 396 |
| HSCP2_T18 (SEQ ID NO: 264) | 1 | 396 |
| HSCP2_T19 (SEQ ID NO: 265) | 1 | 396 |
| HSCP2_T20 (SEQ ID NO: 266) | 1 | 396 |
| HSCP2_T21 (SEQ ID NO: 267) | 1 | 396 |
| HSCP2_T23 (SEQ ID NO: 268) | 1 | 396 |
| HSCP2_T25 (SEQ ID NO: 269) | 1 | 396 |
| HSCP2_T28 (SEQ ID NO: 270) | 1 | 396 |
| HSCP2_T29 (SEQ ID NO: 271) | 1 | 396 |
| HSCP2_T5 (SEQ ID NO: 259) | 1 | 396 |
| HSCP2_T6 (SEQ ID NO: 260) | 1 | 396 |
| HSCP2_T7 (SEQ ID NO: 261) | 1 | 396 |
| HSCP2_T9 (SEQ ID NO: 262) | 1 | 396 |

Segment cluster HSCP2_N3 (SEQ ID NO:273) according to the present invention is supported by 59 libraries. The number of libraries was determined as previously described. This segment can be found in the following transcript(s): HSCP2_T11 (SEQ ID NO:263), HSCP2_T18 (SEQ ID NO:264), HSCP2_T19 (SEQ ID NO:265), HSCP2_T21 (SEQ ID NO:267), HSCP2_T25 (SEQ ID NO:269), HSCP2_T28 (SEQ ID NO:270), HSCP2_T29 (SEQ ID NO:271), HSCP2_T5 (SEQ ID NO:259), HSCP2_T6 (SEQ ID NO:260), HSCP2_T7 (SEQ ID NO:261) and HSCP2_T9 (SEQ ID NO:262). Table 165 below describes the starting and ending position of this segment on each transcript.

TABLE 165
Segment location on transcripts

| | Segment | Segment |
|---|---|---|
| | starting | ending |
| Transcript name | position | position |
| HSCP2_T11 (SEQ ID NO: 263) | 397 | 588 |
| HSCP2_T18 (SEQ ID NO: 264) | 397 | 588 |
| HSCP2_T19 (SEQ ID NO: 265) | 397 | 588 |
| HSCP2_T21 (SEQ ID NO: 267) | 397 | 588 |
| HSCP2_T25 (SEQ ID NO: 269) | 397 | 588 |
| HSCP2_T28 (SEQ ID NO: 270) | 397 | 588 |
| HSCP2_T29 (SEQ ID NO: 271) | 397 | 588 |
| HSCP2_T5 (SEQ ID NO: 259) | 397 | 588 |
| HSCP2_T6 (SEQ ID NO: 260) | 397 | 588 |
| HSCP2_T7 (SEQ ID NO: 261) | 397 | 588 |
| HSCP2_T9 (SEQ ID NO: 262) | 397 | 588 |

Segment cluster HSCP2_N6 (SEQ ID NO:274) according to the present invention is supported by 66 libraries. The number of libraries was determined as previously described. This segment can be found in the following transcript(s): HSCP2_T11 (SEQ ID NO:263), HSCP2_T18 (SEQ ID NO:264), HSCP2_T19 (SEQ ID NO:265), HSCP2_T20 (SEQ ID NO:266), HSCP2_T25 (SEQ ID NO:269), HSCP2_T28 (SEQ ID NO:270), HSCP2_T29 (SEQ ID NO:271), HSCP2_T5 (SEQ ID NO:259), HSCP2_T6 (SEQ ID NO:260), HSCP2_T7 (SEQ ID NO:261) and HSCP2_T9 (SEQ ID NO:262). Table 166 below describes the starting and ending position of this segment on each transcript.

TABLE 166
Segment location on transcripts

| | Segment | Segment |
|---|---|---|
| | starting | ending |
| Transcript name | position | position |
| HSCP2_T11 (SEQ ID NO: 263) | 645 | 831 |
| HSCP2_T18 (SEQ ID NO: 264) | 645 | 831 |
| HSCP2_T19 (SEQ ID NO: 265) | 645 | 831 |
| HSCP2_T20 (SEQ ID NO: 266) | 397 | 583 |
| HSCP2_T25 (SEQ ID NO: 269) | 645 | 831 |
| HSCP2_T28 (SEQ ID NO: 270) | 645 | 831 |
| HSCP2_T29 (SEQ ID NO: 271) | 645 | 831 |
| HSCP2_T5 (SEQ ID NO: 259) | 645 | 831 |
| HSCP2_T6 (SEQ ID NO: 260) | 645 | 831 |
| HSCP2_T7 (SEQ ID NO: 261) | 645 | 831 |
| HSCP2_T9 (SEQ ID NO: 262) | 645 | 831 |

Segment cluster HSCP2_N29 (SEQ ID NO:281) according to the present invention is supported by 76 libraries. The number of libraries was determined as previously described. This segment can be found in the following transcript(s): HSCP2_T11 (SEQ ID NO:263), HSCP2_T18 (SEQ ID NO:264), HSCP2_T19 (SEQ ID NO:265), HSCP2_T20 (SEQ ID NO:266), HSCP2_T21 (SEQ ID NO:267), HSCP2_T23 (SEQ ID NO:268), HSCP2_T25 (SEQ ID NO:269), HSCP2_T28 (SEQ ID NO:270), HSCP2_T5 (SEQ ID NO:259), HSCP2_T6 (SEQ ID NO:260), HSCP2_T7 (SEQ ID NO:261) and HSCP2_T9 (SEQ ID NO:262). Table 167 below describes the starting and ending position of this segment on each transcript.

TABLE 167
Segment location on transcripts

| | Segment | Segment |
|---|---|---|
| | starting | ending |
| Transcript name | position | position |
| HSCP2_T11 (SEQ ID NO: 263) | 1964 | 2114 |
| HSCP2_T18 (SEQ ID NO: 264) | 1964 | 2114 |
| HSCP2_T19 (SEQ ID NO: 265) | 1964 | 2114 |
| HSCP2_T20 (SEQ ID NO: 266) | 1716 | 1866 |
| HSCP2_T21 (SEQ ID NO: 267) | 1577 | 1727 |
| HSCP2_T23 (SEQ ID NO: 268) | 1329 | 1479 |
| HSCP2_T25 (SEQ ID NO: 269) | 1964 | 2114 |
| HSCP2_T28 (SEQ ID NO: 270) | 1964 | 2114 |
| HSCP2_T5 (SEQ ID NO: 259) | 1964 | 2114 |
| HSCP2_T6 (SEQ ID NO: 260) | 1964 | 2114 |
| HSCP2_T7 (SEQ ID NO: 261) | 1964 | 2114 |
| HSCP2_T9 (SEQ ID NO: 262) | 1964 | 2114 |

Segment cluster HSCP2_N30 (SEQ ID NO:282) according to the present invention is supported by 5 libraries. The number of libraries was determined as previously described. This segment can be found in the following transcript(s): HSCP2_T28 (SEQ ID NO:270). Table 168 below describes the starting and ending position of this segment on each transcript.

TABLE 168
Segment location on transcripts

| | Segment | Segment |
|---|---|---|
| | starting | ending |
| Transcript name | position | position |
| HSCP2_T28 (SEQ ID NO: 270) | 2115 | 2247 |

Segment cluster HSCP2_N46 (SEQ ID NO:286) according to the present invention is supported by 9 libraries. The number of libraries was determined as previously described. This segment can be found in the following transcript(s): HSCP2_T25 (SEQ ID NO:269). Table 169 below describes the starting and ending position of this segment on each transcript.

TABLE 169
Segment location on transcripts

| | Segment | Segment |
|---|---|---|
| | starting | ending |
| Transcript name | position | position |
| HSCP2_T25 (SEQ ID NO: 269) | 2805 | 3996 |

Segment cluster HSCP2_N54 (SEQ ID NO:288) according to the present invention is supported by 95 libraries. The number of libraries was determined as previously described. This segment can be found in the following transcript(s): HSCP2_T11 (SEQ ID NO:263), HSCP2_T18 (SEQ ID NO:264), HSCP2_T19 (SEQ ID NO:265), HSCP2_T20 (SEQ ID NO:266), HSCP2_T21 (SEQ ID NO:267), HSCP2_T23 (SEQ ID NO:268), HSCP2_T5 (SEQ ID NO:259), HSCP2_T6 (SEQ ID NO:260), HSCP2_T7 (SEQ ID NO:261) and HSCP2_T9 (SEQ ID NO:262). Table 170 below describes the starting and ending position of this segment on each transcript.

TABLE 170
Segment location on transcripts

| | Segment | Segment |
|---|---|---|
| | starting | ending |
| Transcript name | position | position |
| HSCP2_T11 (SEQ ID NO: 263) | 3129 | 3268 |
| HSCP2_T18 (SEQ ID NO: 264) | 3129 | 3268 |
| HSCP2_T19 (SEQ ID NO: 265) | 2916 | 3055 |
| HSCP2_T20 (SEQ ID NO: 266) | 2881 | 3020 |
| HSCP2_T21 (SEQ ID NO: 267) | 2742 | 2881 |
| HSCP2_T23 (SEQ ID NO: 268) | 2494 | 2633 |
| HSCP2_T5 (SEQ ID NO: 259) | 3129 | 3268 |
| HSCP2_T6 (SEQ ID NO: 260) | 3190 | 3329 |
| HSCP2_T7 (SEQ ID NO: 261) | 3129 | 3268 |
| HSCP2_T9 (SEQ ID NO: 262) | 3129 | 3268 |

Segment cluster HSCP2_N65 (SEQ ID NO:289) according to the present invention is supported by 28 libraries. The number of libraries was determined as previously described. This segment can be found in the following transcript(s): HSCP2_T5 (SEQ ID NO:259). Table 171 below describes the starting and ending position of this segment on each transcript.

TABLE 171
Segment location on transcripts

| | Segment | Segment |
|---|---|---|
| | starting | ending |
| Transcript name | position | position |
| HSCP2_T5 (SEQ ID NO: 259) | 3432 | 5581 |

According to an optional embodiment of the present invention, short segments related to the above cluster are also provided. These segments are up to about 120 bp in length, and so are included in a separate description.

Segment cluster HSCP2_N4 (SEQ ID NO:291) according to the present invention is supported by 57 libraries. The number of libraries was determined as previously described. This segment can be found in the following transcript(s): HSCP2_T11 (SEQ ID NO:263), HSCP2_T18 (SEQ ID NO:264), HSCP2_T19 (SEQ ID NO:265), HSCP2_T21 (SEQ ID NO:267), HSCP2_T25 (SEQ ID NO:269), HSCP2_T28 (SEQ ID NO:270), HSCP2_T29 (SEQ ID NO:271), HSCP2_T5 (SEQ ID NO:259), HSCP2_T6 (SEQ ID NO:260), HSCP2_T7 (SEQ ID NO:261) and HSCP2_T9 (SEQ ID NO:262). Table 172 below describes the starting and ending position of this segment on each transcript.

TABLE 172
Segment location on transcripts

| | Segment | Segment |
|---|---|---|
| | starting | ending |
| Transcript name | position | position |
| HSCP2_T11 (SEQ ID NO: 263) | 589 | 644 |
| HSCP2_T18 (SEQ ID NO: 264) | 589 | 644 |
| HSCP2_T19 (SEQ ID NO: 265) | 589 | 644 |
| HSCP2_T21 (SEQ ID NO: 267) | 589 | 644 |
| HSCP2_T25 (SEQ ID NO: 269) | 589 | 644 |
| HSCP2_T28 (SEQ ID NO: 270) | 589 | 644 |
| HSCP2_T29 (SEQ ID NO: 271) | 589 | 644 |
| HSCP2_T5 (SEQ ID NO: 259) | 589 | 644 |
| HSCP2_T6 (SEQ ID NO: 260) | 589 | 644 |
| HSCP2_T7 (SEQ ID NO: 261) | 589 | 644 |
| HSCP2_T9 (SEQ ID NO: 262) | 589 | 644 |

Segment cluster HSCP2_N13 (SEQ ID NO:293) according to the present invention is supported by 50 libraries. The number of libraries was determined as previously described. This segment can be found in the following transcript(s): HSCP2_T11 (SEQ ID NO:263), HSCP2_T18 (SEQ ID NO:264), HSCP2_T19 (SEQ ID NO:265), HSCP2_T20 (SEQ ID NO:266), HSCP2_T21 (SEQ ID NO:267), HSCP2_T23 (SEQ ID NO:268), HSCP2_T25 (SEQ ID NO:269), HSCP2_T28 (SEQ ID NO:270), HSCP2_T29 (SEQ ID NO:271), HSCP2_T5 (SEQ ID NO:259), HSCP2_T6 (SEQ ID NO:260), HSCP2_T7 (SEQ ID NO:261) and HSCP2_T9 (SEQ ID NO:262). Table 173 below describes the starting and ending position of this segment on each transcript.

TABLE 173
Segment location on transcripts

| | Segment | Segment |
|---|---|---|
| | starting | ending |
| Transcript name | position | position |
| HSCP2_T11 (SEQ ID NO: 263) | 1032 | 1089 |
| HSCP2_T18 (SEQ ID NO: 264) | 1032 | 1089 |
| HSCP2_T19 (SEQ ID NO: 265) | 1032 | 1089 |
| HSCP2_T20 (SEQ ID NO: 266) | 784 | 841 |
| HSCP2_T21 (SEQ ID NO: 267) | 645 | 702 |
| HSCP2_T23 (SEQ ID NO: 268) | 397 | 454 |
| HSCP2_T25 (SEQ ID NO: 269) | 1032 | 1089 |
| HSCP2_T28 (SEQ ID NO: 270) | 1032 | 1089 |
| HSCP2_T29 (SEQ ID NO: 271) | 1032 | 1089 |
| HSCP2_T5 (SEQ ID NO: 259) | 1032 | 1089 |
| HSCP2_T6 (SEQ ID NO: 260) | 1032 | 1089 |
| HSCP2_T7 (SEQ ID NO: 261) | 1032 | 1089 |
| HSCP2_T9 (SEQ ID NO: 262) | 1032 | 1089 |

Segment cluster HSCP2_N24 (SEQ ID NO:296) according to the present invention is supported by 1 libraries. The number of libraries was determined as previously described. This segment can be found in the following transcript(s): HSCP2 T29 (SEQ ID NO:271). Table 174 below describes the starting and ending position of this segment on each transcript.

TABLE 174
Segment location on transcripts

| | Segment | Segment |
|---|---|---|
| | starting | ending |
| Transcript name | position | position |
| HSCP2_T29 (SEQ ID NO: 271) | 1752 | 1779 |

Segment cluster HSCP2_N35 (SEQ ID NO:297) according to the present invention is supported by 59 libraries. The number of libraries was determined as previously described. This segment can be found in the following transcript(s): HSCP2_T11 (SEQ ID NO:263), HSCP2_T18 (SEQ ID NO:264), HSCP2_T19 (SEQ ID NO:265), HSCP2_T20 (SEQ ID NO:266), HSCP2_T21 (SEQ ID NO:267), HSCP2_T23 (SEQ ID NO:268), HSCP2_T25 (SEQ ID NO:269), HSCP2_T5 (SEQ ID NO:259), HSCP2_T6 (SEQ ID NO:260), HSCP2_T7 (SEQ ID NO:261) and HSCP2_T9 (SEQ ID NO:262). Table 1175 below describes the starting and ending position of this segment on each transcript.

TABLE 175
Segment location on transcripts

| | Segment | Segment |
|---|---|---|
| | starting | ending |
| Transcript name | position | position |
| HSCP2_T11 (SEQ ID NO: 263) | 2328 | 2369 |
| HSCP2_T18 (SEQ ID NO: 264) | 2328 | 2369 |
| HSCP2_T19 (SEQ ID NO: 265) | 2115 | 2156 |
| HSCP2_T20 (SEQ ID NO: 266) | 2080 | 2121 |
| HSCP2_T21 (SEQ ID NO: 267) | 1941 | 1982 |
| HSCP2_T23 (SEQ ID NO: 268) | 1693 | 1734 |
| HSCP2_T25 (SEQ ID NO: 269) | 2328 | 2369 |
| HSCP2_T5 (SEQ ID NO: 259) | 2328 | 2369 |
| HSCP2_T6 (SEQ ID NO: 260) | 2328 | 2369 |
| HSCP2_T7 (SEQ ID NO: 261) | 2328 | 2369 |
| HSCP2_T9 (SEQ ID NO: 262) | 2328 | 2369 |

Segment cluster HSCP2_N37 (SEQ ID NO:298) according to the present invention is supported by 18 libraries. The number of libraries was determined as previously described. This segment can be found in the following transcript(s): HSCP2_T6 (SEQ ID NO:260). Table 176 below describes the starting and ending position of this segment on each transcript.

TABLE 176
Segment location on transcripts

| | Segment | Segment |
|---|---|---|
| | starting | ending |
| Transcript name | position | position |
| HSCP2_T6 (SEQ ID NO: 260) | 2536 | 2596 |

Segment cluster HSCP2_N55 (SEQ ID NO:303) according to the present invention is supported by 1 libraries. The number of libraries was determined as previously described. This segment can be found in the following transcript(s): HSCP2_T18 (SEQ ID NO:264). Table 177 below describes the starting and ending position of this segment on each transcript.

TABLE 177
Segment location on transcripts

| | Segment | Segment |
|---|---|---|
| | starting | ending |
| Transcript name | position | position |
| HSCP2_T18 (SEQ ID NO: 264) | 3269 | 3321 |

Segment cluster HSCP2_N61 (SEQ ID NO:305) according to the present invention is supported by 88 libraries. The number of libraries was determined as previously described. This segment can be found in the following transcript(s): HSCP2_T19 (SEQ ID NO:265), HSCP2_T20 (SEQ ID NO:266), HSCP2_T21 (SEQ ID NO:267), HSCP2_T23 (SEQ ID NO:268), HSCP2_T5 (SEQ ID NO:259), HSCP2_T6 (SEQ ID NO:260), HSCP2_T7 (SEQ ID NO:261) and HSCP2_T9 (SEQ ID NO:262). Table 178 below describes the starting and ending position of this segment on each transcript.

TABLE 178
Segment location on transcripts

| | Segment | Segment |
|---|---|---|
| Transcript name | starting position | ending position |
| HSCP2_T19 (SEQ ID NO: 265) | 3061 | 3116 |
| HSCP2_T20 (SEQ ID NO: 266) | 3026 | 3081 |
| HSCP2_T21 (SEQ ID NO: 267) | 2887 | 2942 |
| HSCP2_T23 (SEQ ID NO: 268) | 2639 | 2694 |
| HSCP2_T5 (SEQ ID NO: 259) | 3274 | 3329 |
| HSCP2_T6 (SEQ ID NO: 260) | 3335 | 3390 |
| HSCP2_T7 (SEQ ID NO: 261) | 3274 | 3329 |
| HSCP2_T9 (SEQ ID NO: 262) | 3269 | 3324 |

Segment cluster HSCP2_N62 (SEQ ID NO:306) according to the present invention is supported by 98 libraries. The number of libraries was determined as previously described. This segment can be found in the following transcript(s): HSCP2_T11 (SEQ ID NO:263), HSCP2_T19 (SEQ ID NO:265), HSCP2_T20 (SEQ ID NO:266), HSCP2_T21 (SEQ ID NO:267), HSCP2_T23 (SEQ ID NO:268), HSCP2_T5 (SEQ ID NO:259), HSCP2_T6 (SEQ ID NO:260), HSCP2_T7 (SEQ ID NO:261) and HSCP2_T9 (SEQ ID NO:262). Table 179 below describes the starting and ending position of this segment on each transcript.

TABLE 179
Segment location on transcripts

| | Segment | Segment |
|---|---|---|
| Transcript name | starting position | ending position |
| HSCP2_T11 (SEQ ID NO: 263) | 3269 | 3370 |
| HSCP2_T19 (SEQ ID NO: 265) | 3117 | 3218 |
| HSCP2_T20 (SEQ ID NO: 266) | 3082 | 3183 |
| HSCP2_T21 (SEQ ID NO: 267) | 2943 | 3044 |
| HSCP2_T23 (SEQ ID NO: 268) | 2695 | 2796 |
| HSCP2_T5 (SEQ ID NO: 259) | 3330 | 3431 |
| HSCP2_T6 (SEQ ID NO: 260) | 3391 | 3492 |
| HSCP2_T7 (SEQ ID NO: 261) | 3330 | 3431 |
| HSCP2_T9 (SEQ ID NO: 262) | 3325 | 3426 |

Segment cluster HSCP2_N63 (SEQ ID NO:307) according to the present invention is supported by 11 libraries. The number of libraries was determined as previously described. This segment can be found in the following transcript(s): HSCP2_T7 (SEQ ID NO:261). Table 180 below describes the starting and ending position of this segment on each transcript.

TABLE 180
Segment location on transcripts

| | Segment | Segment |
|---|---|---|
| Transcript name | starting position | ending position |
| HSCP2_T7 (SEQ ID NO: 261) | 3432 | 3443 |

Expression of Homo sapiens ceruloplasmin (ferroxidase) (CP) HSCP2 transcripts which are detectable by amplicon as depicted in sequence name HSCP2_junc0-13 (SEQ ID NO:325) in normal and cancerous Lung tissues

Expression of Homo sapiens ceruloplasmin (ferroxidase) (CP) transcripts detectable by or according to junc0-13—HSCP2_junc0-13 (SEQ ID NO:325) amplicon and primers HSCP2_junc0-13F (SEQ ID NO:323) and HSCP2_junc0-13R (SEQ ID NO:324) was measured by real time PCR. In parallel the expression of four housekeeping genes—HPRT1 (GenBank Accession No. NM_{—}000194 (SEQ ID NO:5); amplicon—HPRT1-amplicon (SEQ ID NO:8), PBGD (GenBank Accession No. BC019323 (SEQ ID NO:1); amplicon—PBGD-amplicon (SEQ ID NO:4), SDHA (GenBank Accession No. NM_{—}004168 (SEQ ID NO:33); amplicon—SDHA-amplicon (SEQ ID NO:36) and Ubiquitin (GenBank Accession No. BC000449 (SEQ ID NO:29); amplicon—Ubiquitin-amplicon (SEQ ID NO:32) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96 and 99, Table 1_{—}3 above), to obtain a value of fold up-regulation for each sample relative to median of the normal PM samples.

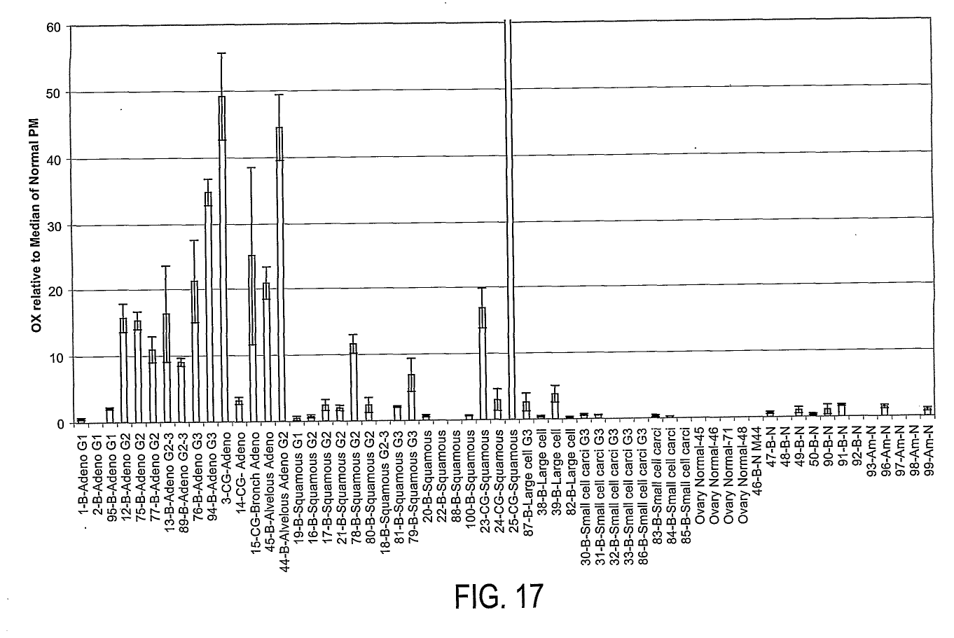

FIG. 17 is a histogram showing over expression of the above-indicated Homo sapiens ceruloplasmin (ferroxidase) (CP) transcripts in cancerous Lung samples relative to the normal samples. Values represent the average of duplicate experiments. Error bars indicate the minimal and maximal values obtained.

As is evident from FIG. 17, the expression of Homo sapiens ceruloplasmin (ferroxidase) (CP) transcripts detectable by the above amplicon in non-small cell carcinoma samples, especially in adenocarcinoma samples was significantly higher than in the non-cancerous samples (sample numbers 47, 48, 49, 50, 90, 91, 92, 93, 96 and 99, Table 1_{—}3 above). Notably an over-expression of at least 5 fold was found in 15 out of 31 non-small cell carcinoma samples, specifically in 11 out of 14 adenocarcinoma samples, and in 4 out of 13 squamous cell carcinoma samples.

Statistical analysis was applied to verify the significance of these results, as described below.

The P value for the difference in the expression levels of Homo sapiens ceruloplasmin (ferroxidase) (CP) transcripts detectable by the above amplicon in Lung adenocarcinoma samples versus the normal tissue samples was determined by T test as 5.63e-004. The P value for the difference in the expression levels of Homo sapiens ceruloplasmin (ferroxidase) (CP) transcripts detectable by the above amplicon in all Lung non-small cell carcinoma samples versus the normal tissue samples was determined by T test as 8.9e-003.

Threshold of 5 fold over expression was found to differentiate between adenocarcinoma and normal samples with P value of 1.3e-003 as checked by exact Fisher test. Threshold of 5 fold over expression was found to differentiate between non-small cell carcinoma and normal samples with P value of 1.94e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HSCP2_junc0-13F (SEQ ID NO:323) forward primer; and HSCP2_junc0-13R (SEQ ID NO:324) reverse primer.

The present invention also preferably encompasses any amplicon obtained through the use of any suitable primer pair; for example, for the above experiment, the following amplicon was obtained as a non-limiting illustrative example only of a suitable amplicon: HSCP2_junc0-13 (SEQ ID NO:325).

Forward Primer (HSCP2_junc0-13F (SEQ ID NO: 323)):
GGAATTATTGAAACGACTTGGGATTA
Reverse Primer (HSCP2_junc0-13R (SEQ ID NO: 324)):
AAAGTGTATCCATTCACAGGTGTCA
Amplicon (HSCP2_junc0-13 (SEQ ID NO: 325)):
GGAATTATTGAAACGACTTGGGATTATGCCTCTGACCATGGGGAAAAGAA
ACTTATTTCTGTTGACACCTGTGAATGGATACACTTT

Expression of Homo sapiens ceruloplasmin (ferroxidase) (CP) HSCP2 transcripts which are detectable by amplicon as depicted in sequence name HSCP2_junc0-13 (SEQ ID NO:325) in different normal tissues

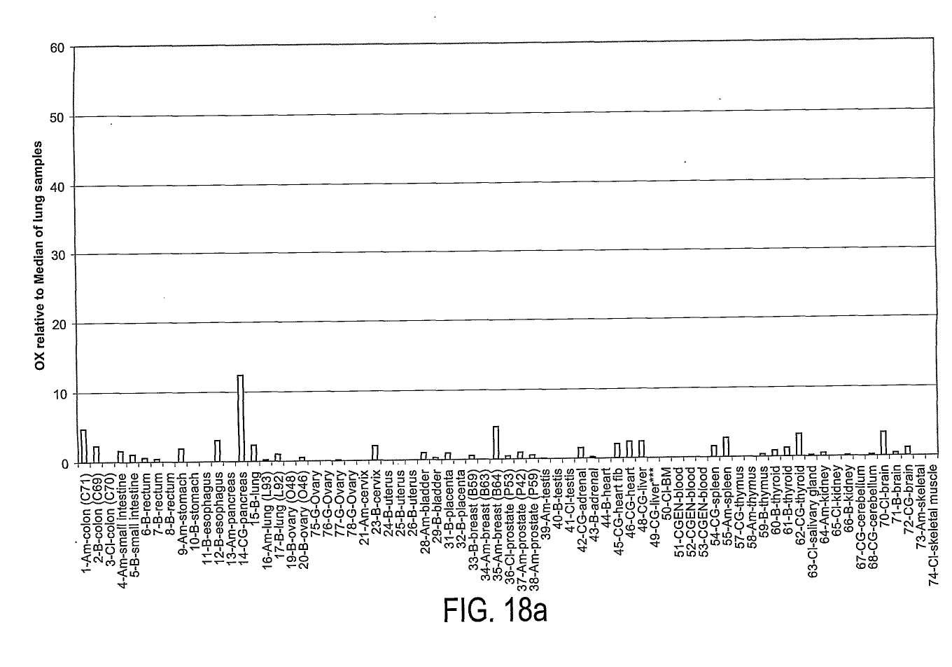

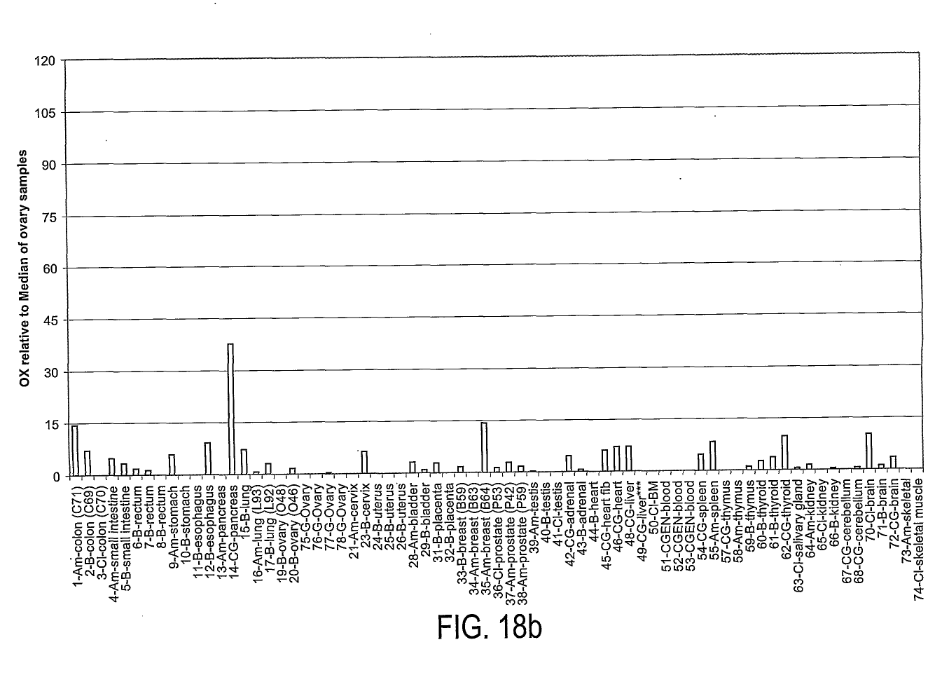

Expression of Homo sapiens ceruloplasmin (ferroxidase) (CP) transcripts detectable by or according to junc0-13—HSCP2_junc0-13 (SEQ ID NO:325) ainplicon and primers HSCP2_junc0-13F (SEQ ID NO:323) and HSCP2_junc0-13R (SEQ ID NO:324) was measured by real time PCR. In parallel the expression of four housekeeping genes—SDHA (GenBank Accession No. NM_{—}004168 (SEQ ID NO:33); amplicon—SDHA-amplicon (SEQ ID NO:36)), Ubiquitin (GenBank Accession No. BC000449 (SEQ ID NO:29); amplicon—Ubiquitin-amplicon (SEQ ID NO:32)), RPL19 (GenBank Accession No. NM_{—}000981 (SEQ ID NO:21); RPL19 amplicon (SEQ ID NO:24)) and TATA box (GenBank Accession No. NM_{—}003194 (SEQ ID NO:25); TATA amplicon (SEQ ID NO:28)) was measured similarly. For each RT sample, the expression of the above amplicon was normalized to the geometric mean of the quantities of the housekeeping genes. The normalized quantity of each RT sample was then divided by either the median of the quantities of the lung samples (sample numbers 15, 16 and 17, Table 1_{—}5 above), to obtain a value of relative expression of each sample relative to median of the lung samples—as presented in FIG. 18a, or by the median of the quantities of the ovary samples (sample numbers 20 and 77 Table 1_{—}5 above), to obtain a value of relative expression of each sample relative to median of the ovary samples—as preasented in FIG. 18b.

Forward Primer (HSCP2_junc0-13F (SEQ ID NO: 323):
GGAATTATTGAAACGACTTGGGATTA
Reverse Primer (HSCP2_junc0-13R (SEQ ID NO: 324):
AAAGTGTATCCATTCACAGGTGTCA
Amplicon (HSCP2_junc0-13 (SEQ ID NO: 325):
GGAATTATTGAAACGACTTGGGATTATGCCTCTGACCATGGGGAAAAGAA
ACTTATTTCTGTTGACACCTGTGAATGGATACACTTT

Expression of Homo sapiens ceruloplasmin (ferroxidase) (CP) HSCP2 transcripts which are detectable by amplicon as depicted in sequence name HSCP2_junc0-13 (SEQ ID NO:325) in normal and cancerous Ovary tissues

Expression of Homo sapiens ceruloplasmin (ferroxidase) (CP) transcripts detectable by or according to junc0-13—HSCP2_Junc0-13 (SEQ ID NO:325) amplicon and primers HSCP2_junc0-13F (SEQ ID NO:323) and HSCP2_junc0-13R (SEQ ID NO:324) was measured by real time PCR. In parallel the expression of four housekeeping genes—SDHA (GenBank Accession No. NM_{—}004168 (SEQ ID NO:33); amplicon—SDHA-amplicon (SEQ ID NO:36), HPRT1 (GenBank Accession No. NM_{—}000194 (SEQ ID NO:5); amplicon—HPRT1-amplicon (SEQ ID NO:8), PBGD (GenBank Accession No. BC019323 (SEQ ID NO:1); amplicon—PBGD-amplicon (SEQ ID NO:4) and GAPDH (GenBank Accession No. BC026907 (SEQ ID NO:9); GAPDH amplicon (SEQ ID NO:12) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 46 and 71, Table 1_{—}1 above), to obtain a value of fold up-regulation for each sample relative to median of the normal PM samples.

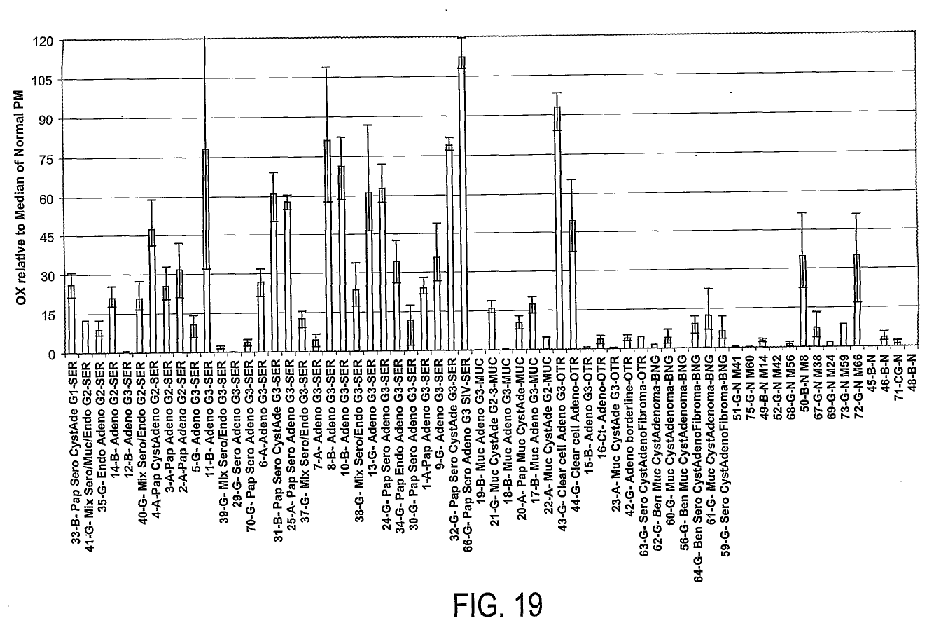

FIG. 19 is a histogram showing over expression of the above-indicated Homo sapiens ceruloplasmin (ferroxidase) (CP) transcripts in cancerous Ovary samples relative to the normal samples. Values represent the average of duplicate experiments. Error bars indicate the minimal and maximal values obtained.

As is evident from FIG. 19, the expression of Homo sapiens ceruloplasmin (ferroxidase) (CP) transcripts detectable by the above amplicon in adenocarcinoma samples was higher than in the non-cancerous samples (sample numbers 46 and 71, Table 1_{—}1 above). Notably an over-expression of at least 5 fold was found in 24 out of 36 adenocarcinoma samples, specifically in 20 out of 27 serous carcinoma samples, and in 2 out of 6 mucinous carcinoma samples.

Statistical analysis was applied to verify the significance of these results, as described below.

The P value for the difference in the expression levels of Homo sapiens ceruloplasmin (ferroxidase) (CP) transcripts detectable by the above amplicon in Ovary serous carcinoma samples versus the normal tissue samples was determined by T test as 1.01e-006. The P value for the difference in the expression levels of Homo sapiens ceruloplasmin (ferroxidase) (CP) transcripts detectable by the above amplicon in Ovary mucinous carcinoma samples versus the normal tissue samples was determined by T test as 8.82e-002. The P value for the difference in the expression levels of Homo sapiens ceruloplasmin (ferroxidase) (CP) transcripts detectable by the above amplicon in Ovary adenocarcinoma samples versus the normal tissue samples was determined by T test as 4.42e-007.

The above values demonstrate statistical significance of the results.

Primer pairs are also optionally and preferably encompassed within the present invention; for example, for the above experiment, the following primer pair was used as a non-limiting illustrative example only of a suitable primer pair: HSCP2_junc0-13F (SEQ ID NO:323) forward primer; and HSCP2_junc0-13R (SEQ ID NO:324) reverse primer.

The present invention also preferably encompasses any amplicon obtained through the use of any suitable primer pair; for example, for the above experiment, the following amplicon was obtained as a non-limiting illustrative example only of a suitable amplicon: HSCP2_junc0-13 (SEQ ID NO:325).

Forward Primer (HSCP2_junc0-13F (SEQ ID NO: 323):
GGAATTATTGAAACGACTTGGGATTA
Reverse Primer (HSCP2_junc0-13R (SEQ ID NO: 324):
AAAGTGTATCCATTCACAGGTGTCA
Amplicon (HSCP2_junc0-13 (SEQ ID NO: 325):
GGAATTATTGAAACGACTTGGGATTATGCCTCTGACCATGGGGAAAAGAA
ACTTATTTCTGTTGACACCTGTGAATGGATACACTTT

Expression of Homo sapiens ceruloplasmin (ferroxidase) (CP) HSCP2 transcripts which are detectable by amplicon as depicted in sequence name HSCP2_seg3WT (SEQ ID NO:328 in normal and cancerous Lung tissues

Expression of Homo sapiens ceruloplasmin (ferroxidase) (CP) transcripts detectable by or according to seg3WT—HSCP2_seg3WT (SEQ ID NO:328 amplicon and primers HSCP2_seg3WTF (SEQ ID NO:326) and HSCP2_seg3WTR (SEQ ID NO:327) was measured by real time PCR. In parallel the expression of four housekeeping genes—HPRT1 (GenBank Accession No. NM_{—}000194 (SEQ ID NO:5); amplicon—HPRT1-amplicon (SEQ ID NO:8), PBGD (GenBank Accession No. BC019323 (SEQ ID NO:1); amplicon—PBGD-amplicon (SEQ ID NO:4), SDHA (GenBank Accession No. NM_{—}004168 (SEQ ID NO:33); amplicon—SDHA-amplicon (SEQ ID NO:36) and Ubiquitin (GenBank Accession No. BC000449 (SEQ ID NO:29); amplicon—Ubiquitin-amplicon (SEQ ID NO:32) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 47, 48, 49, 50, 90, 91, 92, 93, 96, 97, 98 and 99, Table 1_{—}3 above), to obtain a value of fold up-regulation for each sample relative to median of the normal PM samples.

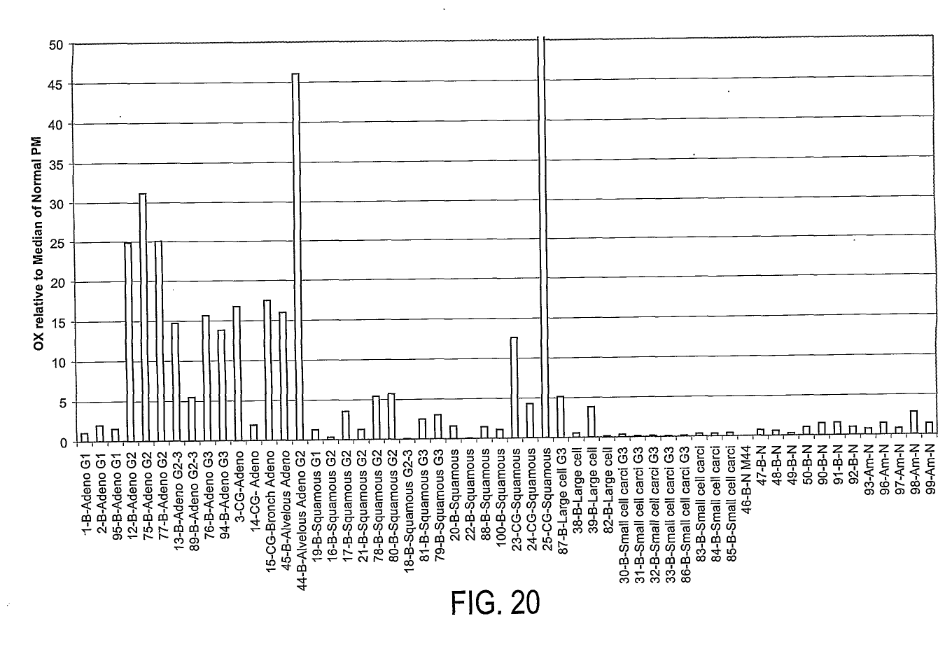

FIG. 20 is a histogram showing over expression of the above-indicated Homo sapiens ceruloplasmin (ferroxidase) (CP) transcripts in cancerous Lung samples relative to the normal samples.

As is evident from FIG. 20, the expression of Homo sapiens ceruloplasmin (ferroxidase) (CP) transcripts detectable by the above amplicon in non-small cell carcinoma samples, especially in adenocarcinoma, was significantly higher than in the non-cancerous samples (sample numbers 47, 48, 49, 50, 90, 91, 92, 93, 96, 97, 98 and 99, Table 1_{—}3 above). Notably an over-expression of at least 5 fold was found in 16 out of 35 non-small cell carcinoma samples, specifically in 11 out of 15 adenocarcinoma samples, and in 4 out of 16 squamous cell carcinoma samples.

Statistical analysis was applied to verify the significance of these results, as described below.

The P value for the difference in the expression levels of Homo sapiens ceruloplasmin (ferroxidase) (CP) transcripts detectable by the above amplicon in Lung adenocarcinoma samples versus the normal tissue samples was determined by T test as 5.92e-004. The P value for the difference in the expression levels of Homo sapiens ceruloplasmin (ferroxidase) (CP) transcripts detectable by the above amplicon in Lung small cell carcinoma samples versus the normal tissue samples was determined by T test as 4.03e-004. The P value for the difference in the expression levels of Homo sapiens ceruloplasmin (ferroxidase) (CP) transcripts detectable by the above amplicon in Lung non-small cell carcinoma samples versus the normal tissue samples was determined by T test as 2.09e-002.

Threshold of 5 fold over expression was found to differentiate between adenocarcinoma and normal samples with P value of 1.05e-004 as checked by exact Fisher test. Threshold of 5 fold over expression was found to differentiate between non-small cell carcinoma and normal samples with P value of 2.70e-003 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HSCP2_seg3WTF (SEQ ID NO:326) forward primer; and HSCP2_seg3WTR (SEQ ID NO:327) reverse primer.

The present invention also preferably encompasses any amplicon obtained through the use of any suitable primer pair; for example, for the above experiment, the following amplicon was obtained as a non-limiting illustrative example only of a suitable amplicon: HSCP2_seg3WT (SEQ ID NO:328.

Forward Primer (HSCP2_seg3WTF (SEQ ID NO: 326):
TCAAAATGGCCCAGATAGAATTG
Reverse Primer (HSCP2_seg3WTR (SEQ ID NO: 327):
GCCAGACCGGTTTTTCTATAGTTGT
Amplicon (HSCP2_seg3WT (SEQ ID NO: 328)):
TCAAAATGGCCCAGATAGAATTGGGAGACTATATAAGAAGGCCCTTTATC
TTCAGTACACAGATGAAACCTTTAGGACAACTATAGAAAAACCGGTCTGG
C

Expression of Homo sapiens ceruloplasmin (ferroxidase) (CP) HSCP2 transcripts which are detectable by amplicon as depicted in sequence name HSCP2_seg3WT (SEQ ID NO:328 in different normal tissues

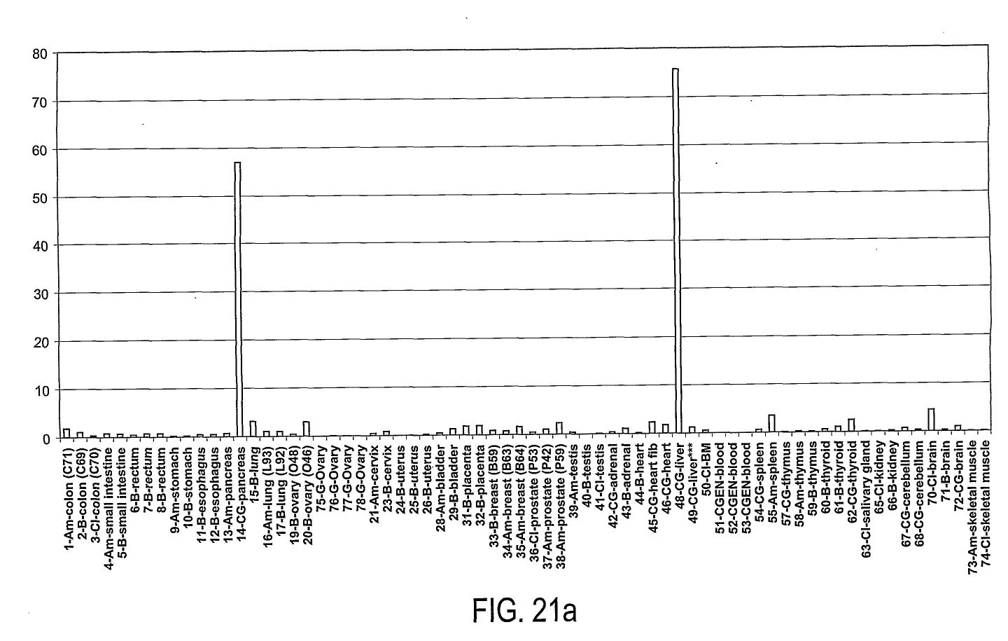

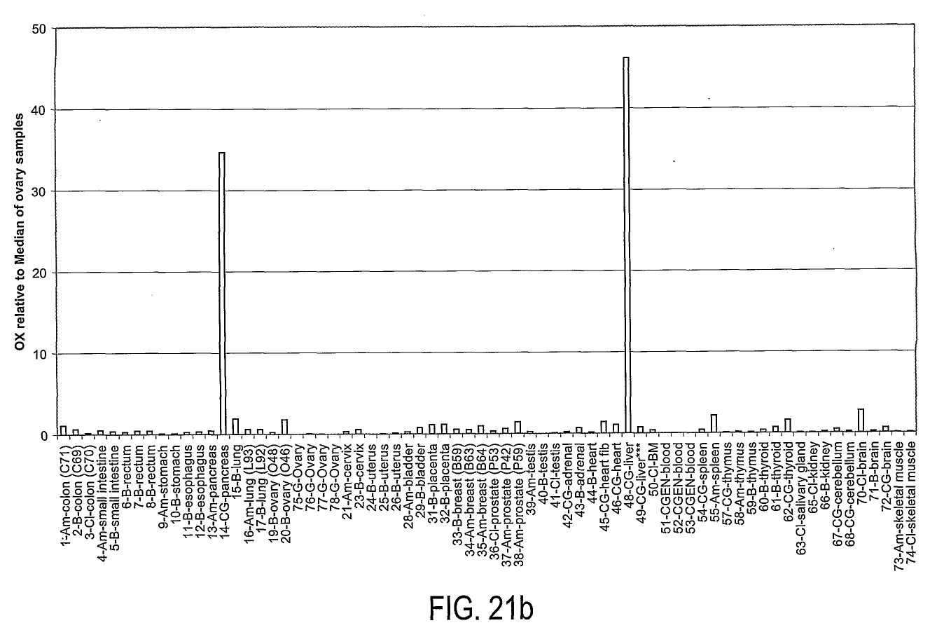

Expression of Homo sapiens ceruloplasmin (ferroxidase) (CP) transcripts detectable by or according to seg3WT—HSCP2_seg3WT (SEQ ID NO:328 amplicon and primers HSCP2_seg3WTF (SEQ ID NO:326) and HSCP2_seg3WTR (SEQ ID NO:327) was measured by real time PCR. In parallel the expression of four housekeeping genes—SDHA (GenBank Accession No. NM_{—}004168 (SEQ ID NO:33); amplicon—SDHA-amplicon (SEQ ID NO:36), Ubiquitin (GenBank Accession No. BC000449 (SEQ ID NO:29); amplicon—Ubiquitin-amplicon (SEQ ID NO:32), RPL19 (GenBank Accession No. NM_{—}000981 (SEQ ID NO:21); RPL19 amplicon (SEQ ID NO:24) and TATA box (GenBank Accession No. NM_{—}003194 (SEQ ID NO:25); TATA amplicon (SEQ ID NO:28) was measured similarly. For each RT sample, the expression of the above amplicon was normalized to the geometric mean of the quantities of the housekeeping genes. The normalized quantity of each RT sample was then divided by either the median of the quantities of the lung samples (sample numbers 15, 16 and 17, Table 1_{—}5 above), to obtain a value of relative expression of each sample relative to median of the lung samples—as presented in FIG. 21a, or by the median of the quantities of the ovary samples (sample numbers 19, 20, Table 1_{—}5 above), to obtain a value of relative expression of each sample relative to median of the ovary samples—as presented in FIG. 21b.

Forward Primer (HSCP2_seg3WTF (SEQ ID NO: 326):
TCAAAATGGCCCAGATAGAATTG
Reverse Primer (HSCP2_seg3WTR (SEQ ID NO: 327):
GCCAGACCGGTTTTTCTATAGTTGT
Amplicon (HSCP2_seg3WT (SEQ ID NO: 328)):
TCAAAATGGCCCAGATAGAATTGGGAGACTATATAAGAAGGCCCTTTATC
TTCAGTACACAGATGAAACCTTTAGGACAACTATAGAAAAACCGGTCTGG
C

Expression of Homo sapiens ceruloplasmin (ferroxidase) (CP) HSCP2 transcripts which are detectable by amplicon as depicted in sequence name HSCP2_seg3WT (SEQ ID NO:328) in normal and cancerous Ovary tissues

Expression of Homo sapiens ceruloplasmin (ferroxidase) (CP) transcripts detectable by or according to seg3WT—HSCP2_seg3WT (SEQ ID NO:328) amplicon and primers HSCP2_seg3WTF (SEQ ID NO:326) and HSCP2_seg3WTR (SEQ ID NO:327) was measured by real time PCR. In parallel the expression of four housekeeping genes—SDHA (GenBank Accession No. NM_{—}004168 (SEQ ID NO:33); amplicon—SDHA-amplicon (SEQ ID NO:36), HPRT1 (GenBank Accession No. NM_{—}000194 (SEQ ID NO:5); amplicon—HPRT1-amplicon (SEQ ID NO:8), PBGD (GenBanK Accession No. BC019323 (SEQ ID NO:1); amplicon—PBGD-amplicon (SEQ ID NO:4) and GAPDH (GenBank Accession No. BC026907 (SEQ ID NO:9); GAPDH amplicon (SEQ ID NO:12) was measured similarly. For each RT sample, the expression of the above amplicon was normalized to the geometric mean of the quantities of the housekeeping genes. The normalized quantity of each RT sample was then divided by the median of the quantities of the normal post-mortem (PM) samples (sample numbers 45, 46, 71 and 48, Table 1_{—}1 above), to obtain a value of fold up-regulation for each sample relative to median of the normal PM samples.

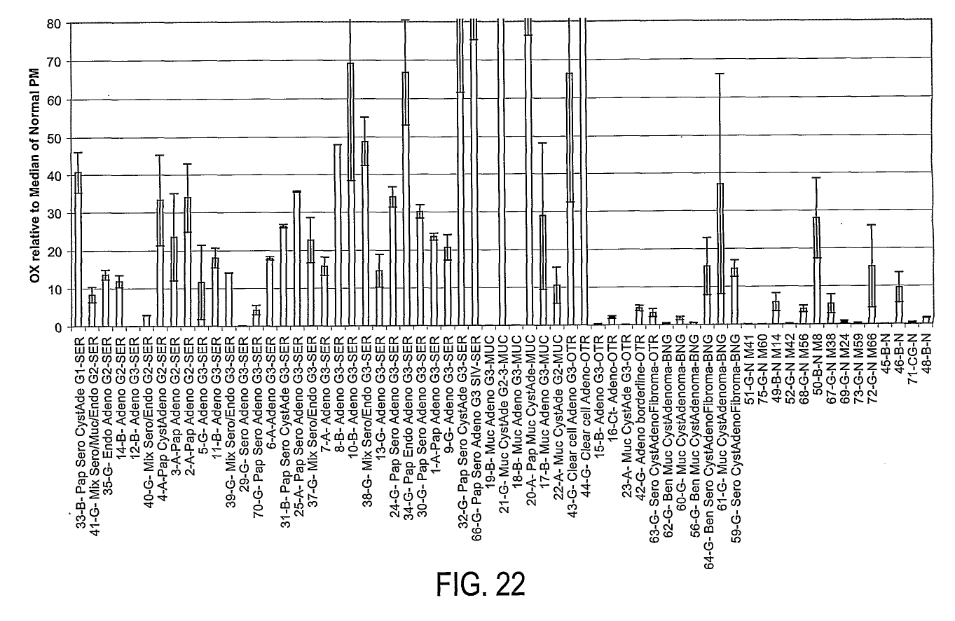

FIG. 22 is a histogram showing over expression of the above-indicated Homo sapiens ceruloplasmin (ferroxidase) (CP) transcripts in cancerous Ovary samples relative to the normal samples. Values represent the average of duplicate experiments. Error bars indicate the minimal and maximal values obtained.

As is evident from FIG. 22, the expression of Homo sapiens ceruloplasmin (ferroxidase) (CP) transcripts detectable by the above amplicon in adenocarcinoma samples was significantly higher than in the non-cancerous samples (sample numbers 45, 46, 71 and 48, Table 1_{—}1 above. Notably an over-expression of at least 5 fold was found in in 32 out of 43 adenocarcinoma samples, specifically in 26 out of 30 serous carcinoma samples, and in 4 out of 6 mucinous carcinoma samples.

Statistical analysis was applied to verify the significance of these results, as described below.

The P value for the difference in the expression levels of Homo sapiens ceruloplasmin (ferroxidase) (CP) transcripts detectable by the above amplicon in Ovary serous carcinoma samples versus the normal tissue samples was determined by T test as 1.58e-005. The P value for the difference in the expression levels of Homo sapiens ceruloplasmin (ferroxidase) (CP) transcripts detectable by the above amplicon in Ovary adenocarcinoma samples versus the normal tissue samples was determined by T test as 1.54e-005.

Threshold of 5 fold over expression was found to differentiate between serous carcinoma and normal samples with P value of 2.09e-002 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: HSCP2_seg3WTF (SEQ ID NO:326) forward primer; and HSCP2_seg3WTR (SEQ ID NO:327) reverse primer.

The present invention also preferably encompasses any amplicon obtained through the use of any suitable primer pair; for example, for the above experiment, the following amplicon was obtained as a non-limiting illustrative example only of a suitable amplicon: HSCP2_seg3WT (SEQ ID NO:328).

Forward Primer (HSCP2_seg3WTF (SEQ ID NO: 326):
TCAAAATGGCCCAGATAGAATTG
Reverse Primer (HSCP2_seg3WTR (SEQ ID NO: 327):
GCCAGACCGGTTTTTCTATAGTTGT
Amplicon (HSCP2_seg3WT (SEQ ID NO: 328):
TCAAAATGGCCCAGATAGAATTGGGAGACTATATAAGAAGGCCCTTTATC
TTCAGTACACAGATGAAACCTTTAGGACAACTATAGAAAAACCGGTCTGG
C

### Description for Cluster S56200

Cluster S56200 features 6 transcript(s) and 26 segment(s) of interest, the names for which are given in Tables 181 and 182, respectively. The selected protein variants are given in table 183.

TABLE 181
Transcripts of interest
Transcript Name

S56200_T3 (SEQ ID NO: 329)
S56200_T5 (SEQ ID NO: 330)
S56200_T7 (SEQ ID NO: 331)
S56200_T8 (SEQ ID NO: 332)
S56200_T10 (SEQ ID NO: 333)
S56200_T11 (SEQ ID NO: 334)

TABLE 182
Segments of interest
Segment Name

S56200_N1 (SEQ ID NO: 335)
S56200_N2 (SEQ ID NO: 336)
S56200_N7 (SEQ ID NO: 337)
S56200_N11 (SEQ ID NO: 338)
S56200_N13 (SEQ ID NO: 339)
S56200_N17 (SEQ ID NO: 340)
S56200_N18 (SEQ ID NO: 341)
S56200_N19 (SEQ ID NO: 342)
S56200_N32 (SEQ ID NO: 343)
S56200_N33 (SEQ ID NO: 344)
S56200_N38 (SEQ ID NO: 345)
S56200_N42 (SEQ ID NO: 346)
S56200_N43 (SEQ ID NO: 347)
S56200_N44 (SEQ ID NO: 348)
S56200_N0 (SEQ ID NO: 349)
S56200_N4 (SEQ ID NO: 350)
S56200_N5 (SEQ ID NO: 351)
S56200_N15 (SEQ ID NO: 352)
S56200_N20 (SEQ ID NO: 353)
S56200_N24 (SEQ ID NO: 354)
S56200_N25 (SEQ ID NO: 355)
S56200_N31 (SEQ ID NO: 356)
S56200_N34 (SEQ ID NO: 357)
S56200_N35 (SEQ ID NO: 358)
S56200_N39 (SEQ ID NO: 359)
S56200_N41 (SEQ ID NO: 360)

TABLE 183
Proteins of interest

| Protein Name | Corresponding Transcript(s) |
|---|---|
| S56200_P6 (SEQ ID NO: 365) | S56200_T7 (SEQ ID NO: 331) |
| S56200_P7 (SEQ ID NO: 366) | S56200_T8 (SEQ ID NO: 332) |
| S56200_P10 (SEQ ID NO: 367) | S56200_T11 (SEQ ID NO: 334) |
| S56200_P21 (SEQ ID NO: 368) | S56200_T3 (SEQ ID NO: 329) |
| S56200_P24 (SEQ ID NO: 369) | S56200_T5 (SEQ ID NO: 330) |
| S56200_P31 (SEQ ID NO: 370) | S56200_T10 (SEQ ID NO: 333) |

These sequences are variants of the known protein Myeloperoxidase precursor (SEQ ID NO:361) (SwissProt accession identifier PERM_HUMAN (SEQ ID NO:371); known also according to the synonyms EC 1.11.1.7; MPO), referred to herein as the previously known protein.

Myeloperoxidase is part of the host defense system of human polymorphonuclear leukocytes. In the stimulated PMN, MPO catalyzes the production of hypohalous acids, primarily hypochlorous acid in physiologic situations, and other toxic intermediates that greatly enhance PMN microbicidal activity. MPO-derived oxidants contribute to tissue damage during inflammation. MPO is located in the nucleus as well as in the cytoplasm. Intranuclear MPO may help to protect DNA against damage resulting from oxygen radicals produced during myeloid cell maturation and function. The primary step in the classification of granules in neutrophils is according to content of MPO.

Myeloperoxidase-catalyzed reactions have been attributed to potentially proatherogenic biological activities throughout the evolution of cardiovascular disease, including during initiation, propagation, and acute complication phases of the atherosclerotic process. As a result, myeloperoxidase and its downstream inflammatory pathways represent attractive targets for both prognostication and therapeutic intervention in the prophylaxis of atherosclerotic cardiovascular disease (Myeloperoxidase and Cardiovascular Disease Am J. Cardiol. 2006; 98(11A):9P-17P).

Known polymorphisms for Myeloperoxidase precursor (SEQ ID NO:361)sequence are as shown in Table 184.

TABLE 184
Amino acid mutations for Known Protein

| SNP position(s) on | |
|---|---|
| amino acid sequence | Comment |
| 173 | Y -> C (in MPD; affects proteolytic processing |
| | and secretion). /FTId = VAR_015377 |
| 251 | M -> T (in MPD). /FTId = VAR_015378 |
| 569 | R -> W (in MPD; suppress posttranslational |
| | processing). /FTId = VAR_015379 |
| 717 | I -> V (in dbSNP: 2759). /FTId = VAR_012066 |
| 36 | L -> V |

The variant S56200_P24 (SEQ ID NO:369) was previously disclosed by the inventors in published PCT application no WO2005/071058 and U.S. application Ser. No. 11/043,860. The variant S56200_P10 (SEQ ID NO:367) was previously disclosed by the inventors in published PCT application no WO2004/096979, WO2005/071058 and U.S. application Ser. Nos. 10/242,799, 10/426,002, 10/873,314, 11/043,860, hereby incorporated by reference as if fully set forth herein. The variants S56200_P24 (SEQ ID NO:369) and S56200_P10 (SEQ ID NO:367) have now been shown to have novel and surprising diagnostic uses as described herein for other variants of cluster S56200.

According to optional but preferred embodiments of the present invention, variants of this cluster according to the present invention (amino acid and/or nucleic acid sequences of S56200) may optionally have one or more of the following utilities, as described in greater detail below. It should be noted that these utilities are optionally and preferably suitable for human and non-human animals as subjects, except where otherwise noted. The reasoning is described with regard to biological and/or physiological and/or other information about the known protein, but is given to demonstrate particular diagnostic utility for the variants according to the present invention.

A non-limiting example of such a utility is the determination of efficacy of prophylaxis therapy for myocardial infarction by measuring the level of MPO. The method comprises detecting a S56200 variant, for example a variant protein, protein fragment, peptide, polynucleotide, polynucleotide fragment and/or oligonucleotide as described herein, optionally and preferably in a serum sample. The expression levels of the S56200 variant as determined in a patient can be further compared to those in a normal individual.

The above use of the known Myeloperoxidase is described with regard to PCT Application No. WO 05/075022, hereby incorporated by reference as if fully set forth herein.

Another non-limiting example of such a utility is the determination of risk of developing cardiac disease by measuring MPO levels. The method comprises detecting a S56200 variant, for example a variant protein, protein fragment, peptide, polynucleotide, polynucleotide fragment and/or oligonucleotide as described herein, optionally and preferably in a serum sample. The expression levels of the S56200 variant as determined in a patient can be further compared to those in a normal individual.

The above use of the known Myeloperoxidase is described with regard to PCT Application No. WO 02/062207, hereby incorporated by reference as if fully set forth herein.

Another non-limiting example of such a utility is the detection or diagnosis of pauci-immune necrotizing and/or crescentic glomerulonephritis by measuring MPO levels. The method comprises detecting a S56200 variant, for example a variant protein, protein fragment, peptide, polynucleotide, polynucleotide fragment and/or oligonucleotide as described herein, optionally and preferably in a serum sample. The expression levels of the S56200 variant as determined in a patient can be further compared to those in a normal individual.

The above use of the known Myeloperoxidase is described with regard to U.S. Pat. No. 5,200,319, hereby incorporated by reference as if fully set forth herein.

Another non-limiting example of such a utility is the determination or diagnosis of acute myocardial infarction by measuring MPO levels. The method comprises detecting a S56200 variant, for example a variant protein, protein fragment, peptide, polynucleotide, polynucleotide fragment and/or oligonucleotide as described herein, optionally and preferably in a serum sample. The expression levels of the S56200 variant as determined in a patient can be further compared to those in a normal individual.

The above use of the known Myeloperoxidase is described with regard to PCT Application No. WO 05/041893, hereby incorporated by reference as if fully set forth herein.

Another non-limiting example of such a utility is the determination of risk of arterial plaque rupture and thrombus formation by measuring MPO levels. The method comprises detecting a S56200 variant, for example a variant protein, protein fragment, peptide, polynucleotide, polynucleotide fragment and/or oligonucleotide as described herein, optionally and preferably in a serum sample. The expression levels of the S56200 variant as determined in a patient can be further compared to those in a normal individual.

The above use of the known Myeloperoxidase is described with regard to PCT Application No. WO 05/045440, hereby incorporated by reference as if fully set forth herein, as an example of a proximal inflammatory marker. Proximal inflammatory markers are in particular associated with the risk that plaques already present in an individual will undergo inflammation, augmenting the probability of plaque rupture and thrombus formation.

Another non-limiting example of the utilities of amino acid and/or nucleic acid sequences of S56200 variants acording to the present invention is determination of lung cancer risk. The reasoning is described with regard to information about the known protein, correlating bewteen polymorphisms and lung cancer (Wu X M, et al., Ai Zheng. 2003 September; 22(9):912-5; Larsen J E, et al., Carcinogenesis. 2006 March; 27(3):525-32. Epub 2005 September 29; Kiyohara C, et al., Genet Med. 2005 September; 7(7):463-78), but is given to demonstrate this particular diagnostic utility for the variants according to the present invention.

Another non-limiting example of the utilities of amino acid and/or nucleic acid sequences of S56200 variants acording to the present invention is determination of Alzheimer's risk. The reasoning is described with regard to information about the known protein, correlating between genetic polymorphism and Alzheimer's risk (Experimental neurology 1999, vol. 155, no 1, pp. 31-41), but is given to demonstrate this particular diagnostic utility for the variants according to the present invention.

According to optional but preferred embodiments of the present invention, variants of this cluster according to the present invention (amino acid and/or nucleic acid sequences of S56200) may optionally have one or more of the following utilities, as described with regard to Table 185 below. The reasoning is described with regard to biological and/or physiological and/or other information about the known protein, but is given to demonstrate particular diagnostic utility for the variants according to the present invention.

TABLE 185

| Indication | Reason | reference |
|---|---|---|
| Diagnosis and prediction of | Initial plasma | N Engl J Med. 2003 Oct |
| myocardial infraction. | myeloperoxidase levels | 23; 349(17): 1595-604. |
| | predicted the risk of | |
| | myocardial infarction, and of | |
| | major adverse cardiac events | |
| | even in patients who are | |
| | negative for troponin T. | |
| | Plasma levels of the enzyme | Am J Cardiol. 2006 Dec |
| | myeloperoxidase, which | 4; 98(11A): 9P-17P. |
| | generates the strong oxidizing | |
| | agent hypochlorous acid, have | |
| | been found to be correlated | |
| | with risk for myocardial | |
| | infarction and endothelial | |
| | dysfunction. | |
| Molecular imaging of | In the future, novel reporter | J Mol Cell Cardiol. |
| myocardial infarction | agents and high-resolution | 2006 Dec; 41(6): 921-33. |
| | cardiac imaging systems | |
| | should enable imaging of | |
| | emerging targets such as | |
| | activated macrophages and | |
| | myeloperoxidase activity, as | |
| | well as stem cell-based and | |
| | gene therapy-based | |
| | myocardial regenerative | |
| | strategies | |
| Diagnosis of AML and AML | The enzyme myeloperoxidase | Leuk Lymphoma. 1994 |
| subtypes | (MPO) is the most specific | Jan; 12(3-4): 233-9 |
| | marker of myeloid lineage. | |
| | The recognition of acute | |
| | myeloid leukaemia (AML) | |
| | with minimally differentiation | |
| | (AML-M0) is established with | |
| | methods that include myeloid | |
| | markers CD13/CD33 and | |
| | detection of MPO. | |
| | The difference in the survival | Leuk Res. 2006 |
| | of MPO High expressing cells | Sep; 30(9): 1105-12. |
| | and MPO Low expressing cells | |
| | was statistically meaningful, | |
| | demonstrating the possible | |
| | prognostic impact of | |
| | the expression of MPO gene in | |
| | AC133 positive leukemia | |
| | cells. | |
| | In situ hybridization (ISH) was | Diagn Mol Pathol. 1995 |
| | used to evaluate MPO gene | Sep; 4(3): 212-9 |
| | expression in myeloid | J Clin Pathol. 2007 |
| | leukemia cell lines and a | Jan; 60(1): 57-61. |
| | variety of well-characterized | |
| | acute leukemias, including six | |
| | cases of AML-M0. Strong | |
| | positivity for MPO mRNA | |
| | was detected in the myeloid | |
| | leukemia cell line HL-60 and | |
| | in 22 of 27 AMLs | |
| Elevated risk to Alzheimer's | The increase in neuronal | Neurology. 2000 Nov |
| disease (AD) due to MPO | myeloperoxidase expression | 14; 55(9): 1284-90 |
| expression and polymorphism | observed in Alzheimer disease | |
| | brains raises the possibility | |
| | that the enzyme contributes to | |
| | the oxidative stress implicated | |
| | in the pathogenesis of the | |
| | neurodegenerative disorder. | |
| | An MPO promoter | Exp Neurol. 1999 |
| | polymorphism (−463G/A) | Jan; 155(1): 31-41 |
| | linked to increased MPO | |
| | expression has been associated | |
| | with increased risk of AD. | |
| | MPO A enhances AD risk by | |
| | 3.8-fold. | |
| | A significantly higher | J Neurochem. 2004 |
| | percentage of male patients | Aug; 90(3): 724-33 |
| | with AD carried the MPO A | |
| | and APOE epsilon4 alleles | |
| | relative to men carrying | |
| | neither allele (p < 0.001; OR, | |
| | 11.4; 95% CI, 3.6 to 6.7). | |
| | Male APOE epsilon4 carriers | Exp Neurol. 2001 |
| | lacking the MPO A allele had | Feb; 167(2): 456-9 |
| | an OR of 3.0 (p = 0.01; 95% | |
| | CI, 1.3 to 6.9), indicating that | |
| | MPO A enhances AD risk by | |
| | 3.8-fold. | |
| | Specifically, the MPO GG | |
| | genotype contributes a 1.57- | |
| | fold increased risk for AD. | |
| Antineutrophil cytoplasmic | Antineutrophil cytoplasmic | Acta Dermatovenerol Croat. |
| antibodies (ANCA) the | antibodies (ANCA) are a | 2004; 12(4): 294-313 |
| pathogenesis of vasculitis. | heterogeneous group of | Nat Clin Pract Rheumatol. |
| | circulating antibodies directed | 2006 Dec; 2(12): 661-70 |
| | toward the cytoplasmic | |
| | constituents of neutrophils and | |
| | monocytes. ANCA recognize | |
| | different target antigens such | |
| | as proteinase 3 (PR3-ANCA), | |
| | myeloperoxidase (MPO- | |
| | ANCA). | |

As noted above, cluster S56200 features 6 transcript(s), which were listed in Table 181 above. These transcript(s) encode for protein(s) which are variant(s) of protein Myeloperoxidase precursor (SEQ ID NO:361). A description of each variant protein according to the present invention is now provided.

Variant protein S56200_P6 (SEQ ID NO:365) according to the present invention is encoded by transcript S56200_T7 (SEQ ID NO:331) One or more alignments to one or more previously published protein sequences are given in the alignment table on the attached CD-ROM. A brief description of the relationship of the variant protein according to the present invention to each such aligned protein is as follows:

1. Comparison report between S56200_P6 (SEQ ID NO:365) and PERM_HUMAN (SEQ ID NO:371):

A. An isolated chimeric polypeptide encoding for S56200_P6 (SEQ ID NO:365), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNRRSPTLGASNRAFVRWLPAEYEDGFSLPYGWTPGVKRNGFPVALARAVSNEIV RFPTDQLTPDQERSLMFMQWGQLLDHDLDFTPEPAARASFVTGVNCETSCVQQPPCFPL KIPPNDPRfKNQADCIPFFRSCPACPGSNITIRNQINALTSFVDASMVYGSEEPLARNLRNM SNQLGLLAVNQRFQDNGRALLPFDNLHDDPCLLTNRSARIPCFLAGDTRSSEMPELTSMH TLLLREHNRLATELKSLNPRWDGERLYQEARKIVGAMVQIITYRDYLPLVLGPTAMRKY LPTYRSYNDSVDPRIANVFTNAFRYGHTLIQPFMFRLDNRYQPMEPNPRVPLSRVFFASW RVVLEGGIDPILRGLMATPAKLNRQNQIAVDEIRERLFEQVMRIGLDLPALNMQRSRDHG LPGYNAWRRFCGLPQPETVGQLGTVLRNLKLARKLMEQYGTPNNIDIWMGGVSEPLKR KGRVGPLLACIIG corresponding to amino acids 1-666 of PERM_HUMAN (SEQ ID NO:371), which also corresponds to amino acids 1-666 of S56200_P6 (SEQ ID NO:365), and a second amino acid sequence being at least 70%, optionally at least 80%, preferably at least 85%, more preferably at least 90% and most preferably at least 95% homologous to a polypeptide having the sequence FGGRTRVCSACSSDRPWPRSHCPGSSATTQASPPCLRTTSSCPTHIPGTLSTAVHFLH (SEQ ID NO:426) corresponding to amino acids 667-724 of S56200_P6 (SEQ ID NO:365), wherein said first amino acid sequence and second amino acid sequence are contiguous and in a sequential order.

B. An isolated polypeptide encoding for an edge portion of S56200_P6 (SEQ ID NO:365), comprising an amino acid sequence being at least 70%, optionally at least about 80%, preferably at least about 85%, more preferably at least about 90% and most preferably at least about 95% homologous to the sequence FGGRTRVCSACSSDRPWPRSHCPGSSATTQASPPCLRTTSSCPTHIPGTLSTAVHFLH (SEQ ID NO:426) of S56200_P6 (SEQ ID NO:365).

3. Comparison report between S56200_P6 (SEQ ID NO:365) and P05164-3 (SEQ ID NO:364):

A. An isolated chimeric polypeptide encoding for S56200_P6 (SEQ ID NO:365), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNR corresponding to amino acids 1-183 of P05164-3 (SEQ ID NO:364), which also corresponds to amino acids 1-183 of S56200_P6 (SEQ ID NO:365), a second amino acid sequence being at least 90% homologous to RSPTLGASNRAFVRWLPAEYEDGFSLPYGWTPGVKRNGFPVALARAVSNEIVRFPTDQL TPDQERSLMFMQWGQLLDHDLDFTPEPAARASFVTGVNCETSCVQQPPCFPLKIPPNDPR IKNQADCIPFFRSCPACPGSNITIRNQINALTSFVDASMVYGSEEPLARNLRNMSNQLGLL AVNQRFQDNGRALLPFDNLHDDPCLLTNRSARIPCFLAGDTRSSEMPELTSMHTLLLREH NRLATELKSLNPRWDGERLYQEARKIVGAMVQIITYRDYLPLVLGPTAMRKYLPTYRSY NDSVDPRIANVFTNAFRYGHTLIQPFMFRLDNRYQPMEPNPRVPLSRVFFASWRVVLEG GIDPILRGLMATPAKLNRQNQIAVDEIRERLFEQVMRIGLDLPALNMQRSRDHGLPGYNA WRRFCGLPQPETVGQLGTVLRNLKLARKLMEQYGTPNNIDIWMGGVSEPLKRKGRVGP LLACIIG corresponding to amino acids 216-698 of P05164-3 (SEQ ID NO:364), which also corresponds to amino acids 184-666 of S56200_P6 (SEQ ID NO:365), and a third amino acid sequence being at least 70%, optionally at least 80%, preferably at least 85%, more preferably at least 90% and most preferably at least 95% homologous to a polypeptide having the sequence FGGRTRVCSACSSDRPWPRSHCPGSSATTQASPPCLRTTSSCPTHIPGTLSTAVHFLH (SEQ ID NO:426) corresponding to amino acids 667-724 of S56200_P6 (SEQ ID NO:365), wherein said first amino acid sequence, second amino acid sequence and third amino acid sequence are contiguous and in a sequential order.

B. An isolated chimeric polypeptide encoding for an edge portion of S56200_P6 (SEQ ID NO:365), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RR, having a structure as follows: a sequence starting from any of amino acid numbers 183-x to 183; and ending at any of amino acid numbers 184+((n−2)−x), in which x varies from 0 to n−2.

C. An isolated polypeptide encoding for an edge portion of S56200_P6 (SEQ ID NO:365), comprising an amino acid sequence being at least 70%, optionally at least about 80%, preferably at least about 85%, more preferably at least about 90% and most preferably at least about 95% homologous to the sequence FGGRTRVCSACSSDRPWPRSHCPGSSATTQASPPCLRTTSSCPTHIPGTLSTAVHFLH (SEQ ID NO:426) of S56200_P6 (SEQ ID NO:365).

4. Comparison report between S56200_P6 (SEQ ID NO:365) and P05164-2 (SEQ ID NO:362):

A. An isolated chimeric polypeptide encoding for S56200_P6 (SEQ ID NO:365), comprising a first amino acid sequence being at least 70%, optionally at least 80%, preferably at least 85%, more preferably at least 90% and most preferably at least 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) corresponding to amino acids 1-95 of S56200_P6 (SEQ ID NO:365), a second amino acid sequence being at least 90% homologous to MELLSYFKQPVAATRTAVRAADYLHVALDLLERKLRSLWRRPFNVTDVLTPAQLNVLS KSSGCAYQDVGVTCPEQDKYRTITGMCNNRRSPTLGASNRAFVRWLPAEYEDGFSLPYG WTPGVKRNGFPVALARAVSNEIVRFPTDQLTPDQERSLMFMQWGQLLDHDLDFTPEPA ARASFVTGVNCETSCVQQPPCFPLKIPPNDPRIKNQADCIPFFRSCPACPGSNITIRNQINAL TSFVDASMVYGSEEPLARNLRNMSNQLGLLAVNQRFQDNGRALLPFDNLHDDPCLLTN RSARIPCFLAGDTRSSEMPELTSMHTLLLREHNRLATELKSLNPRWDGERLYQEARKIVG AMVQIITYRDYLPLVLGPTAMRKYLPTYRSYNDSVDPRIANVFTNAFRYGHTLIQPFMFR LDNRYQPMEPNPRVPLSRVFFASWRVVLEGGIDPILRGLMATPAKLNRQNQIAVDEIRER LFEQVMRIGLDLPALNMQRSRDHGLPGYNAWRRFCGLPQPETVGQLGTVLRNLKLARK LMEQYGTPNNIDIWMGGVSEPLKRKGRVGPLLACIIG corresponding to amino acids 1-571 of P05164-2 (SEQ ID NO:362), which also corresponds to amino acids 96-666 of S56200_P6 (SEQ ID NO:365), and a third amino acid sequence being at least 70%, optionally at least 80%, preferably at least 85%, more preferably at least 90% and most preferably at least 95% homologous to a polypeptide having the sequence FGGRTRVCSACSSDRPWPRSHCPGSSATTQASPPCLRTTSSCPTHIPGTLSTAVHFLH (SEQ ID NO:426) corresponding to amino acids 667-724 of S56200_P6 (SEQ ID NO:365), wherein said first amino acid sequence, second amino acid sequence and third amino acid sequence are contiguous and in a sequential order.

B. An isolated polypeptide encoding for a head of S56200_P6 (SEQ ID NO:365), comprising a polypeptide being at least 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) of S56200_P6 (SEQ ID NO:365).

C. An isolated polypeptide encoding for an edge portion of S56200_P6 (SEQ ID NO:365), comprising an amino acid sequence being at least 70%, optionally at least about 80%, preferably at least about 85%, more preferably at least about 90% and most preferably at least about 95% homologous to the sequence FGGRTRVCSACSSDRPWPRSHCPGSSATTQASPPCLRTTSSCPTHIPGTLSTAVHFLH (SEQ ID NO:426) of S56200_P6 (SEQ ID NO:365).

The localization of the variant protein was determined according to results from a number of different software programs and analyses, including analyses from SignalP and other specialized programs. The variant protein is believed to be located as follows with regard to the cell: secreted.

Variant protein S56200_P6 (SEQ ID NO:365) also has the following non-silent SNPs (Single Nucleotide Polymorphisms) as listed in Table 186, (given according to their position(s) on the amino acid sequence, with the alternative amino acid(s) listed).

TABLE 186
Amino acid mutations

| SNP position(s) on amino acid | |
|---|---|
| sequence | Alternative amino acid(s) |
| 36 | L -> P |
| 36 | L -> R |
| 53 | V -> F |
| 312 | F -> S |
| 330 | I -> T |
| 514 | N -> H |
| 514 | N -> Y |
| 592 | R -> G |
| 682 | P -> |

The glycosylation sites of variant protein S56200_P6 (SEQ ID NO:365), as compared to the known protein Myeloperoxidase precursor (SEQ ID NO:361), are described in Table 187 (given according to their position(s) on the amino acid sequence in the first column; the second column indicates whether the glycosylation site is present in the variant protein; and the last column indicates whether the position is different on the variant protein).

TABLE 187
Glycosylation site(s)

| Position(s) on known amino | | Position(s) on |
|---|---|---|
| acid sequence | Present in variant protein? | variant protein |
| 355 | Yes | 355 |
| 391 | Yes | 391 |
| 483 | Yes | 483 |

The phosphorylation sites of variant protein S56200_P6 (SEQ ID NO:365), as compared to the known protein, are described in Table 188 (given according to their position(s) on the amino acid sequence in the first column; the second column indicates whether the phosphorylation site is present in the variant protein; and the last column indicates whether the position is different on the variant protein).

TABLE 188
Phosphorylation site(s)

| Position(s) on known amino | | Position(s) on |
|---|---|---|
| acid sequence | Present in variant protein? | variant protein |
| 316 | Yes | 316 |

The variant protein has the following domains, as determined by using InterPro. The domains are described in Table 189:

TABLE 189
InterPro domain(s)

| Domain description | Analysis type | Position(s) on protein |
|---|---|---|
| Animal haem peroxidase | FPrintScan | 197-208, 251-266, 399-417, |
| | | 417-437, 442-468, |
| | | 495-505, 623-643 |
| Animal haem peroxidase | HMMPfam | 171-723 |
| Animal haem peroxidase | Profile Scan | 164-724 |
| Haem peroxidase, plant | ScanRegExp | 408-418 |

Variant protein S56200_P6 (SEQ ID NO:365) is encoded by the transcript S56200_T7 (SEQ ID NO:331), for which the coding portion starts at position 196 and ends at position 2367. The transcript also has the following SNPs as listed in Table 190 (given according to their position on the nucleotide sequence, with the alternative nucleic acid listed).

TABLE 190
Nucleic acid SNPs

| SNP position(s) on nucleotide | |
|---|---|
| sequence | Alternative nucleic acid(s) |
| 171 | G -> A |
| 300 | G -> C |
| 302 | T -> G |
| 302 | T -> C |
| 352 | G -> T |
| 1130 | T -> C |
| 1184 | T -> C |
| 1608 | C -> T |
| 1735 | A -> C |
| 1735 | A -> T |
| 1857 | T -> C |
| 1969 | A -> G |
| 2241 | C -> |
| 2310 | A -> G |
| 2510 | A -> C |
| 2552 | C -> T |
| 2960 | T -> A |

Variant protein S56200_P7 (SEQ ID NO:366) according to the present invention is encoded by transcript S56200_T8 (SEQ ID NO:332) One or more alignments to one or more previously published protein sequences are given in the alignment table on the attached CD-ROM. A brief description of the relationship of the variant protein according to the present invention to each such aligned protein is as follows:

1. Comparison report between S56200_P7 (SEQ ID NO:366) and PERM_HUMAN (SEQ ID NO:371):

A. An isolated chimeric polypeptide encoding for S56200_P7 (SEQ ID NO:366), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNRRSPTLGASNRAFVRWLPAEYEDGFSLPYGWTPGVKRNGFPVALARAVSNEIV RFPTDQLTPDQERSLMFMQWGQLLDHDLDFTPEPAARASFVTGVNCETSCVQQPPCFPL KIPPNDPRIKNQADCIPFFRSCPACPGSNITIRNQINALTSFVDASMVYGSEEPLARNLRNM SNQLGLLAVNQRFQDNGRALLPFDNLHDDPCLLTNRSARIPCFLAGDTRSSEMPELTSMH TLLLREHNRLATELKSLNPRWDGERLYQEARKIVGAMVQIITYRDYLPLVLGPTAMRKY LPTYRSYNDSVDPRIANVFTNAFRYGHTLIQPFMFRLDNRYQPMEPNPRVPLSRVFFASW RVVLEG corresponding to amino acids 1-541 of PERM_HUMAN (SEQ ID NO:371), which also corresponds to amino acids 1-541 of S56200_P7 (SEQ ID NO:366), and a second amino acid sequence being at least 70%, optionally at least 80%, preferably at least 85%, more preferably at least 90% and most preferably at least 95% homologous to a polypeptide having the sequence KQDLGQERVG (SEQ ID NO:428) corresponding to amino acids 542-551 of S56200_P7 (SEQ ID NO:366), wherein said first amino acid sequence and second amino acid sequence are contiguous and in a sequential order.

B. An isolated polypeptide encoding for an edge portion of S56200_P7 (SEQ ID NO:366), comprising an amino acid sequence being at least 70%, optionally at least about 80%, preferably at least about 85%, more preferably at least about 90% and most preferably at least about 95% homologous to the sequence KQDLGQERVG (SEQ ID NO:428) of S56200_P7 (SEQ ID NO:366).

3. Comparison report between S56200_P7 (SEQ ID NO:366) and P05164-3 (SEQ ID NO:364):

A. An isolated chimeric polypeptide encoding for S56200_P7 (SEQ ID NO:366), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNR corresponding to amino acids 1-183 of P05164-3 (SEQ ID NO:364), which also corresponds to amino acids 1-183 of S56200_P7 (SEQ ID NO:366), a second amino acid sequence being at least 90% homologous to RSPTLGASNRAFVRWLPAEYEDGFSLPYGWTPGVKRNGFPVALARAVSNEIVRFPTDQL TPDQERSLMFMQWGQLLDHDLDFTPEPAARASFVTGVNCETSCVQQPPCFPLKIPPNDPR IKNQADCIPFFRSCPACPGSNITIRNQINALTSFVDASMVYGSEEPLARNLRNMSNQLGLL AVNQRFQDNGRALLPFDNLHDDPCLLTNRSARIPCFLAGDTRSSEMPELTSMHTLLLREH NRLATELKSLNPRWDGERLYQEARKIVGAMVQIITYRDYLPLVLGPTAMRKYLPTYRSY NDSVDPRIANVFTNAFRYGHTLIQPFMFRLDNRYQPMEPNPRVPLSRVFFASWRWLEG corresponding to amino acids 216-573 of P05164-3 (SEQ ID NO:364), which also corresponds to amino acids 184-541 of S56200_P7 (SEQ ID NO:366), and a third amino acid sequence being at least 70%, optionally at least 80%, preferably at least 85%, more preferably at least 90% and most preferably at least 95% homologous to a polypeptide having the sequence KQDLGQERVG (SEQ ID NO:428) corresponding to amino acids 542-551 of S56200_P7 (SEQ ID NO:366), wherein said first amino acid sequence, second amino acid sequence and third amino acid sequence are contiguous and in a sequential order.

B. An isolated chimeric polypeptide encoding for an edge portion of S56200_P7 (SEQ ID NO:366), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RR, having a structure as follows: a sequence starting from any of amino acid numbers 183-x to 183; and ending at any of amino acid numbers 184+((n−2)−x), in which x varies from 0 to n−2.

C. An isolated polypeptide encoding for an edge portion of S56200_P7 (SEQ ID NO:366), comprising an amino acid sequence being at least 70%, optionally at least about 80%, preferably at least about 85%, more preferably at least about 90% and most preferably at least about 95% homologous to the sequence KQDLGQERVG (SEQ ID NO:428) of S56200_P7 (SEQ ID NO:366).

4. Comparison report between S56200_P7 (SEQ ID NO:366) and P05164-2 (SEQ ID NO:362):

A. An isolated chimeric polypeptide encoding for S56200_P7 (SEQ ID NO:366), comprising a first amino acid sequence being at least 70%, optionally at least 80%, preferably at least 85%, more preferably at least 90% and most preferably at least 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) corresponding to amino acids 1-95 of S56200_P7 (SEQ ID NO:366), a second amino acid sequence being at least 90% homologous to MELLSYFKQPVAATRTAVRAADYLHVALDLLERKLRSLWRRPFNVTDVLTPAQLNVLS KSSGCAYQDVGVTCPEQDKYRTITGMCNNRRSPTLGASNRAFVRWLPAEYEDGFSLPYG WTPGVKRNGFPVALARAVSNEIVRFPTDQLTPDQERSLMFMQWGQLLDHDLDFTPEPA ARASFVTGVNCETSCVQQPPCFPLKIPPNDPRIKNQADCIPFFRSCPACPGSNITIRNQINAL TSFVDASMVYGSEEPLARNLRNMSNQLGLLAVNQRFQDNGRALLPFDNLHDDPCLLTN RSARIPCFLAGDTRSSEMPELTSMHTLLLREHNRLATELKSLNPRWDGERLYQEARKIVG AMVQIITYRDYLPLVLGPTAMRKYLPTYRSYNDSVDPRIANVFTNAFRYGHTLIQPFMFR LDNRYQPMEPNPRVPLSRVFFASWRVVLEG corresponding to amino acids 1-446 of P05164-2 (SEQ ID NO:362), which also corresponds to amino acids 96-541 of S56200_P7 (SEQ ID NO:366), and a third amino acid sequence being at least 70%, optionally at least 80%, preferably at least 85%, more preferably at least 90% and most preferably at least 95% homologous to a polypeptide having the sequence KQDLGQERVG (SEQ ID NO:428) corresponding to amino acids 542-551 of S56200_P7 (SEQ ID NO:366), wherein said first amino acid sequence, second amino acid sequence and third amino acid sequence are contiguous and in a sequential order.

B. An isolated polypeptide encoding for a head of S56200_P7 (SEQ ID NO:366), comprising a polypeptide being at least 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) of S56200_P7 (SEQ ID NO:366).

C. An isolated polypeptide encoding for an edge portion of S56200_P7 (SEQ ID NO:366), comprising an amino acid sequence being at least 70%, optionally at least about 80%, preferably at least about 85%, more preferably at least about 90% and most preferably at least about 95% homologous to the sequence KQDLGQERVG (SEQ ID NO:428) of S56200_P7 (SEQ ID NO:366).

The localization of the variant protein was determined according to results from a number of different software programs and analyses, including analyses from SignalP and other specialized programs. The variant protein is believed to be secreted.

Variant protein S56200_P7 (SEQ ID NO:366) also has the following non-silent SNPs (Single Nucleotide Polymorphisms) as listed in Table 191, (given according to their position(s) on the amino acid sequence, with the alternative amino acid(s) listed).

TABLE 191
Amino acid mutations

| SNP position(s) on amino acid | |
|---|---|
| sequence | Alternative amino acid(s) |
| 36 | L -> P |
| 36 | L -> R |
| 53 | V -> F |
| 312 | F -> S |
| 330 | I -> T |
| 514 | N -> H |
| 514 | N -> Y |

The glycosylation sites of variant protein S56200_P7 (SEQ ID NO:366), as compared to the known protein Myeloperoxidase precursor (SEQ ID NO:361), are described in Table 192 (given according to their position(s) on the amino acid sequence in the first column; the second column indicates whether the glycosylation site is present in the variant protein; and the last column indicates whether the position is different on the variant protein).

TABLE 192
Glycosylation site(s)

| Position(s) on known amino | | Position(s) on |
|---|---|---|
| acid sequence | Present in variant protein? | variant protein |
| 355 | Yes | 355 |
| 391 | Yes | 391 |
| 483 | Yes | 483 |

The phosphorylation sites of variant protein S56200_P7 (SEQ ID NO:366), as compared to the known protein, are described in Table 193 (given according to their position(s) on the amino acid sequence in the first column; the second column indicates whether the phosphorylation site is present in the variant protein; and the last column indicates whether the position is different on the variant protein).

TABLE 193
Phosphorylation site(s)

| Position(s) on known amino | | Position(s) on |
|---|---|---|
| acid sequence | Present in variant protein? | variant protein |
| 316 | Yes | 316 |

The variant protein has the following domains, as determined by using InterPro. The domains are described in Table 194:

TABLE 194
InterPro domain(s)

| Domain description | Analysis type | Position(s) on protein |
|---|---|---|
| Animal haem peroxidase | FPrintScan | 197-208, 251-266, 399-417, |
| | | 417-437, 442-468, 495-505 |
| Animal haem peroxidase | HMMPfam | 171-551 |
| Animal haem peroxidase | ProfileScan | 164-551 |
| Haem peroxidase, plant | ScanRegExp | 408-418 |

Variant protein S56200_P7 (SEQ ID NO:366) is encoded by the transcript S56200_T8 (SEQ ID NO:332), for which the coding portion starts at position 196 and ends at position 1848. The transcript also has the following SNPs as listed in Table 195 (given according to their position on the nucleotide sequence, with the alternative nucleic acid listed).

TABLE 195
Nucleic acid SNPs

| SNP position(s) on nucleotide | |
|---|---|
| sequence | Alternative nucleic acid(s) |
| 171 | G -> A |
| 300 | G -> C |
| 302 | T -> G |
| 302 | T -> C |
| 352 | G -> T |
| 1130 | T -> C |
| 1184 | T -> C |
| 1608 | C -> T |
| 1735 | A -> C |
| 1735 | A -> T |
| 1909 | C -> T |
| 2352 | T -> C |
| 2464 | A -> G |
| 2770 | C -> |
| 2839 | A -> G |
| 3039 | A -> C |
| 3081 | C -> T |
| 3489 | T -> A |

Variant protein S56200_P10 (SEQ ID NO:367) according to the present invention is encoded by transcript S56200_T11 (SEQ ID NO:334) One or more alignments to one or more previously published protein sequences are given in the alignment table on the attached CD-ROM. A brief description of the relationship of the variant protein according to the present invention to each such aligned protein is as follows:

1. Comparison report between S56200_P10 (SEQ ID NO:367) and PERM_HUMAN (SEQ ID NO:371):

A. An isolated chimeric polypeptide encoding for S56200_P10 (SEQ ID NO:367), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNRRSPTLGASNRAFVRWLPAEYEDGFSLPYGWTPGVKRNGFPVALARAVSNEIV RFPTDQLTPDQERSLMFMQWGQLLDHDLDFTPEPAARASFVTGVNCETSCVQQPPCFPL KIPPNDPRIKNQADCIPFFRSCPACPGSNITIRNQINALTSFVDASMVYGSEEPLAR NRNM SNQLGLLAVNQRFQDNGRALLPFDNLHDDPCLLTNRSARIPCFLA corresponding to amino acids 1-401 of PERM_HUMAN (SEQ ID NO:371), which also corresponds to amino acids 1-401 of S56200_P10 (SEQ ID NO:367), and a second amino acid sequence being at least 70%, optionally at least 80%, preferably at least 85%, more preferably at least 90% and most preferably at least 95% homologous to a polypeptide having the sequence DHHLPGLPAPGAGANGHEEVPAHVPFLQ (SEQ ID NO:430) corresponding to amino acids 402-429 of S56200_P10 (SEQ ID NO:367), wherein said first amino acid sequence and second amino acid sequence are contiguous and in a sequential order.

B. An isolated polypeptide encoding for an edge portion of S56200_P10 (SEQ ID NO:367), comprising an amino acid sequence being at least 70%, optionally at least about 80%, preferably at least about 85%, more preferably at least about 90% and most preferably at least about 95% homologous to the sequence DHHLPGLPAPGAGANGHEEVPAHVPFLQ (SEQ ID NO:430) of S56200_P10 (SEQ ID NO:367).

3. Comparison report between S56200_P10 (SEQ ID NO:367) and P05164-3 (SEQ ID NO:364):

A. An isolated chimeric polypeptide encoding for S56200_P10 (SEQ ID NO:367), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNR corresponding to amino acids 1-183 of P05164-3 (SEQ ID NO:364), which also corresponds to amino acids 1-183 of S56200_P10 (SEQ ID NO:367), a second amino acid sequence being at least 90% homologous to RSPTLGASNRAFVRWLPAEYEDGFSLPYGWTPGVKRNGFPVALARAVSNEIVRFPTDQL TPDQERSLMFMQWGQLLDHDLDFTPEPAARASFVTGVNCETSCVQQPPCFPLKIPPNDPR IKNQADCIPFFRSCPACPGSNITIRNQINALTSFVDASMVYGSEEPLARNLRNMSNQLGLL AVNQRFQDNGRALLPFDNLHDDPCLLTNRSARIPCFLA corresponding to amino acids 216-433 of P05164-3 (SEQ ID NO:364), which also corresponds to amino acids 184-401 of S56200_P10 (SEQ ID NO:367), and a third amino acid sequence being at least 70%, optionally at least 80%, preferably at least 85%, more preferably at least 90% and most preferably at least 95% homologous to a polypeptide having the sequence DHHLPGLPAPGAGANGHEEVPAHVPFLQ (SEQ ID NO:430) corresponding to amino acids 402-429 of S56200_P10 (SEQ ID NO:367), wherein said first amino acid sequence, second amino acid sequence and third amino acid sequence are contiguous and in a sequential order.

B. An isolated chimeric polypeptide encoding for an edge portion of S56200_P10 (SEQ ID NO:367), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RR, having a structure as follows: a sequence starting from any of amino acid numbers 183-x to 183; and ending at any of amino acid numbers 184+((n−2)−x), in which x varies from 0 to n−2.

C. An isolated polypeptide encoding for an edge portion of S56200_P10 (SEQ ID NO:367), comprising an amino acid sequence being at least 70%, optionally at least about 80%, preferably at least about 85%, more preferably at least about 90% and most preferably at least about 95% homologous to the sequence DHHLPGLPAPGAGANGHEEVPAHVPFLQ (SEQ ID NO:430) of S56200_P10 (SEQ ID NO:367).

4. Comparison report between S56200_P10 (SEQ ID NO:367) and P05164-2 (SEQ ID NO:362):

A. An isolated chimeric polypeptide encoding for S56200_P10 (SEQ ID NO:367), comprising a first amino acid sequence being at least 70%, optionally at least 80%, preferably at least 85%, more preferably at least 90% and most preferably at least 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) corresponding to amino acids 1-95 of S56200_P10 (SEQ ID NO:367), a second amino acid sequence being at least 90% homologous to MELLSYFKQPVAATRTAVRAADYLHVALDLLERKLRSLWRRPFNVTDVLTPAQLNVLS KSSGCAYQDVGVTCPEQDKYRTITGMCNNRRSPTLGASNRAFVRWLPAEYEDGFSLPYG WTPGVKRNGFPVALARAVSNEIVRFPTDQLTPDQERSLMFMQWGQLLDHDLDFTPEPA ARASFVTGVNCETSCVQQPPCFPLKIPPNDPRIKNQADCIPFFRSCPACPGSNITIRNQINAL TSFVDASMVYGSEEPLARNLRNMSNQLGLLAVNQRFQDNGRALLPFDNLHDDPCLLTN RSARIPCFLA corresponding to amino acids 1-306 of P05164-2 (SEQ ID NO:362), which also corresponds to amino acids 96-401 of S56200_P10 (SEQ ID NO:367), and a third amino acid sequence being at least 70%, optionally at least 80%, preferably at least 85%, more preferably at least 90% and most preferably at least 95% homologous to a polypeptide having the sequence DHHLPGLPAPGAGANGHEEVPAHVPFLQ (SEQ ID NO:430) corresponding to amino acids 402-429 of S56200_P10 (SEQ ID NO:367), wherein said first amino acid sequence, second amino acid sequence and third amino acid sequence are contiguous and in a sequential order.

B. An isolated polypeptide encoding for a head of S56200_P10 (SEQ ID NO:367), comprising a polypeptide being at least 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) of S56200_P10 (SEQ ID NO:367).

C. An isolated polypeptide encoding for an edge portion of S56200_P10 (SEQ ID NO:367), comprising an amino acid sequence being at least 70%, optionally at least about 80%, preferably at least about 85%, more preferably at least about 90% and most preferably at least about 95% homologous to the sequence DHHLPGLPAPGAGANGHEEVPAHVPFLQ (SEQ ID NO:430) of S56200_P10 (SEQ ID NO:367).

The localization of the variant protein was determined according to results from a number of different software programs and analyses, including analyses from SignalP and other specialized programs. The variant protein is believed to be secreted.

Variant protein S56200_P10 (SEQ ID NO:367) also has the following non-silent SNPs (Single Nucleotide Polymorphisms) as listed in Table 196, (given according to their position(s) on the amino acid sequence, with the alternative amino acid(s) listed).

TABLE 196
Amino acid mutations

| SNP position(s) on amino acid | |
|---|---|
| sequence | Alternative amino acid(s) |
| 36 | L -> P |
| 36 | L -> R |
| 53 | V -> F |
| 312 | F -> S |
| 330 | I -> T |
| 418 | H -> Y |

The glycosylation sites of variant protein S56200_P10 (SEQ ID NO:367), as compared to the known protein Myeloperoxidase precursor (SEQ ID NO:361), are described in Table 197 (given according to their position(s) on the amino acid sequence in the first column; the second column indicates whether the glycosylation site is present in the variant protein; and the last column indicates whether the position is different on the variant protein).

TABLE 197
Glycosylation site(s)

| Position(s) on known amino | | Position(s) on |
|---|---|---|
| acid sequence | Present in variant protein? | variant protein |
| 355 | Yes | 355 |
| 391 | Yes | 391 |
| 483 | No | |

The phosphorylation sites of variant protein S56200_P10 (SEQ ID NO:367), as compared to the known protein, are described in Table 198 (given according to their position(s) on the amino acid sequence in the first column; the second column indicates whether the phosphorylation site is present in the variant protein; and the last column indicates whether the position is different on the variant protein).

TABLE 198
Phosphorylation site(s)

| Position(s) on known amino | | Position(s) on |
|---|---|---|
| acid sequence | Present in variant protein? | variant protein |
| 316 | Yes | 316 |

The variant protein has the following domains, as determined by using InterPro. The domains are described in Table 199:

TABLE 199
InterPro domain(s)

| Domain description | Analysis type | Position(s) on protein |
|---|---|---|
| Animal haem peroxidase | FPrintScan | 197-208, 251-266 |
| Animal haem peroxidase | HMMPfam | 171-427 |
| Animal haem peroxidase | ProfileScan | 164-429 |

Variant protein S56200_P10 (SEQ ID NO:367) is encoded by the transcript S56200_T11 (SEQ ID NO:334), for which the coding portion starts at position 196 and ends at position 1482. The transcript also has the following SNPs as listed in Table 200 (given according to their position on the nucleotide sequence, with the alternative nucleic acid listed).

TABLE 200
Nucleic acid SNPs

| SNP position(s) on nucleotide | |
|---|---|
| sequence | Alternative nucleic acid(s) |
| 171 | G -> A |
| 300 | G -> C |
| 302 | T -> G |
| 302 | T -> C |
| 352 | G -> T |
| 1130 | T -> C |
| 1184 | T -> C |
| 1447 | C -> T |
| 1574 | A -> C |
| 1574 | A -> T |
| 1696 | T -> C |
| 1808 | A -> G |
| 2114 | C -> |
| 2183 | A -> G |
| 2383 | A -> C |
| 2425 | C -> T |
| 2833 | T -> A |

Variant protein S56200_P21 (SEQ ID NO:368) according to the present invention is encoded by transcript S56200_T3 (SEQ ID NO:329). An alignment is given to the known protein (Myeloperoxidase precursor (SEQ ID NO:361) at the end of the application. One or more alignments to one or more previously published protein sequences are given in the alignment table on the attached CD-ROM. A brief description of the relationship of the variant protein according to the present invention to each such aligned protein is as follows:

1. Comparison report between S56200_P21 (SEQ ID NO:368) and PERM_HUMAN (SEQ ID NO:371):

A. An isolated chimeric polypeptide encoding for S56200_P21 (SEQ ID NO:368), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNRRSPTLGASNRAFVRWLPAEYEDGFSLPYGWTPGVKRNGFPVALARAVSNEIV RFPTDQLTPDQERSLMFMQWGQLLDHDLDFTPEPAARASFVTGVNCETSCVQQPPCFPL KIPPNDPRIKNQADCIPFFRSCPACPGSNITIRNQINALTSFVDASMVYGSEEPLARNLRNM SNQLGLLAVNQRFQDNGRALLPFDNLHDDPCLLTNRSARIPCFLAG corresponding to amino acids 1-402 of PERM_HUMAN (SEQ ID NO:371), which also corresponds to amino acids 1-402 of S56200_P21 (SEQ ID NO:368), and a second amino acid sequence being at least 70%, optionally at least 80%, preferably at least 85%, more preferably at least 90% and most preferably at least 95% homologous to a polypeptide having the sequence QLWGGDQRWHRRCSLLEPQGHPFQ (SEQ ID NO:432) corresponding to amino acids 403-426 of S56200_P21 (SEQ ID NO:368), wherein said first amino acid sequence and second amino acid sequence are contiguous and in a sequential order.

B. An isolated polypeptide encoding for an edge portion of S56200_P21 (SEQ ID NO:368), comprising an amino acid sequence being at least 70%, optionally at least about 80%, preferably at least about 85%, more preferably at least about 90% and most preferably at least about 95% homologous to the sequence QLWGGDQRWHRRCSLLEPQGHPFQ (SEQ ID NO:432) of S56200_P21 (SEQ ID NO:368).

3. Comparison report between S56200_P21 (SEQ ID NO:368) and P05164-3 (SEQ ID NO:364):

A. An isolated chimeric polypeptide encoding for S56200_P21 (SEQ ID NO:368), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNR corresponding to amino acids 1-183 of P05164-3 (SEQ ID NO:364), which also corresponds to amino acids 1-183 of S56200_P21 (SEQ ID NO:368), a second amino acid sequence being at least 90% homologous to RSPTLGASNRAFVRWLPAEYEDGFSLPYGWTPGVKRNGFPVALARAVSNEIVRFPTDQL TPDQERSLMFMQWGQLLDHDLDFTPEPAARASFVTGVNCETSCVQQPPCFPLKIPPNDPR IKNQADCIPFFRSCPACPGSNITIRNQINALTSFVDASMVYGSEEPLARNLRNMSNQLGLL AVNQRFQDNGRALLPFDNLHDDPCLLTNRSARIPCFLAG corresponding to amino acids 216-434 of P05164-3 (SEQ ID NO:364), which also corresponds to amino acids 184-402 of S56200_P21 (SEQ ID NO:368), and a third amino acid sequence being at least 70%, optionally at least 80%, preferably at least 85%, more preferably at least 90% and most preferably at least 95% homologous to a polypeptide having the sequence QLWGGDQRWHRRCSLLEPQGHPFQ (SEQ ID NO:432) corresponding to amino acids 403-426 of S56200_P21 (SEQ ID NO:368), wherein said first amino acid sequence, second amino acid sequence and third amino acid sequence are contiguous and in a sequential order.

B. An isolated chimeric polypeptide encoding for an edge portion of S56200_P21 (SEQ ID NO:368), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RR, having a structure as follows: a sequence starting from any of amino acid numbers 183-x to 183; and ending at any of amino acid numbers 184+((n−2)−x), in which x varies from 0 to n−2.

C. An isolated polypeptide encoding for an edge portion of S56200_P21 (SEQ ID NO:368), comprising an amino acid sequence being at least 70%, optionally at least about 80%, preferably at least about 85%, more preferably at least about 90% and most preferably at least about 95% homologous to the sequence QLWGGDQRWHRRCSLLEPQGHPFQ (SEQ ID NO:432) of S56200_P21 (SEQ ID NO:368).

4. Comparison report between S56200_P21 (SEQ ID NO:368) and P05164-2 (SEQ ID NO:362):

A. An isolated chimeric polypeptide encoding for S56200_P21 (SEQ ID NO:368), comprising a first amino acid sequence being at least 70%, optionally at least 80%, preferably at least 85%, more preferably at least 90% and most preferably at least 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) corresponding to amino acids 1-95 of S56200_P21 (SEQ ID NO:368), a second amino acid sequence being at least 90% homologous to MELLSYFKQPVAATRTAVRAADYLHVALDLLERKLRSLWRRPFNVTDVLTPAQLNVLS KSSGCAYQDVGVTCPEQDKYRTITGMCNNRRSPTLGASNRAFVRWLPAEYEDGFSLPYG WTPGVKRNGFPVALARAVSNEIVRFPTDQLTPDQERSLMFMQWGQLLDHDLDFTPEPA ARASFVTGVNCETSCVQQPPCFPLKIPPNDPRIKNQADCIPFFRSCPACPGSNITIRNQINAL TSFVDASMVYGSEEPLARNLRNMSNQLGLLAVNQRFQDNGRALLPFDNLHDDPCLLTN RSARIPCFLAG corresponding to amino acids 1-307 of P05164-2 (SEQ ID NO:362), which also corresponds to amino acids 96-402 of S56200_P21 (SEQ ID NO:368), and a third amino acid sequence being at least 70%, optionally at least 80%, preferably at least 85%, more preferably at least 90% and most preferably at least 95% homologous to a polypeptide having the sequence QLWGGDQRWHRRCSLLEPQGHPFQ (SEQ ID NO:432) corresponding to amino acids 403-426 of S56200_P21 (SEQ ID NO:368), wherein said first amino acid sequence, second amino acid sequence and third amino acid sequence are contiguous and in a sequential order.

B. An isolated polypeptide encoding for a head of S56200_P21 (SEQ ID NO:368), comprising a polypeptide being at least 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) of S56200_P21 (SEQ ID NO:368).

C. An isolated polypeptide encoding for an edge portion of S56200_P21 (SEQ ID NO:368), comprising an amino acid sequence being at least 70%, optionally at least about 80%, preferably at least about 85%, more preferably at least about 90% and most preferably at least about 95% homologous to the sequence QLWGGDQRWHRRCSLLEPQGHPFQ (SEQ ID NO:432) of S56200_P21 (SEQ ID NO:368).

The localization of the variant protein was determined according to results from a number of different software programs and analyses, including analyses from SignalP and other specialized programs. The variant protein is believed to be secreted.

Variant protein S56200_P21 (SEQ ID NO:368) also has the following non-silent SNPs (Single Nucleotide Polymorphisms) as listed in Table 201, (given according to their position(s) on the amino acid sequence, with the alternative amino acid(s) listed).

TABLE 201
Amino acid mutations

| SNP position(s) on amino acid | |
|---|---|
| sequence | Alternative amino acid(s) |
| 36 | L -> P |
| 36 | L -> R |
| 53 | V -> F |
| 312 | F -> S |
| 330 | I -> T |

The glycosylation sites of variant protein S56200_P21 (SEQ ID NO:368), as compared to the known protein Myeloperoxidase precursor (SEQ ID NO:361), are described in Table 202 (given according to their position(s) on the amino acid sequence in the first column; the second column indicates whether the glycosylation site is present in the variant protein; and the last column indicates whether the position is different on the variant protein).

TABLE 202
Glycosylation site(s)

| Position(s) on known amino | | Position(s) on |
|---|---|---|
| acid sequence | Present in variant protein? | variant protein |
| 355 | Yes | 355 |
| 391 | Yes | 391 |
| 483 | No | |

The phosphorylation sites of variant protein S56200_P21 (SEQ ID NO:368), as compared to the known protein, are described in Table 203 (given according to their position(s) on the amino acid sequence in the first column; the second column indicates whether the phosphorylation site is present in the variant protein; and the last column indicates whether the position is different on the variant protein).

TABLE 203
Phosphorylation site(s)

| Position(s) on known amino | | Position(s) on |
|---|---|---|
| acid sequence | Present in variant protein? | variant protein |
| 316 | Yes | 316 |

The variant protein has the following domains, as determined by using InterPro. The domains are described in Table 204:

TABLE 204
InterPro domain(s)

| Domain description | Analysis type | Position(s) on protein |
|---|---|---|
| Animal haem peroxidase | FPrintScan | 197-208, 251-266 |
| Animal haem peroxidase | HMMPfam | 171-426 |
| Animal haem peroxidase | ProfileScan | 164-426 |

Variant protein S56200_P21 (SEQ ID NO:368) is encoded by the transcript S56200_T3 (SEQ ID NO:329), for which the coding portion starts at position 196 and ends at position 1473. The transcript also has the following SNPs as listed in Table 205 (given according to their position on the nucleotide sequence, with the alternative nucleic acid listed).

TABLE 205
Nucleic acid SNPs

| SNP position(s) on nucleotide | |
|---|---|
| sequence | Alternative nucleic acid(s) |
| 171 | G -> A |
| 300 | G -> C |
| 302 | T -> G |
| 302 | T -> C |
| 352 | G -> T |
| 1130 | T -> C |
| 1184 | T -> C |
| 1666 | C -> T |
| 1793 | A -> C |
| 1793 | A -> T |
| 1915 | T -> C |
| 2027 | A -> G |
| 2333 | C -> |
| 2402 | A -> G |
| 2602 | A -> C |
| 2644 | C -> T |
| 3052 | T -> A |

Variant protein S56200_P24 (SEQ ID NO:369) according to the present invention is encoded by transcript S56200_T5 (SEQ ID NO:330) One or more alignments to one or more previously published protein sequences are given in the alignment table on the attached CD-ROM. A brief description of the relationship of the variant protein according to the present invention to each such aligned protein is as follows:

1. Comparison report between S56200_P24 (SEQ ID NO:369) and PERM_HUMAN (SEQ ID NO:371):

A. An isolated chimeric polypeptide encoding for S56200_P24 (SEQ ID NO:369), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNRRSPTLGASNRAFVRWLPAEYEDGFSLPYGWTPGVKRNGFPVALARAVSNEIV RFPTDQLTPDQERSLMFMQWGQLLDHDLDFTPEPAARASFVTGVNCETSCVQQPPCFPL K corresponding to amino acids 1-295 of PERM_HUMAN (SEQ ID NO:371), which also corresponds to amino acids 1-295 of S56200_P24 (SEQ ID NO:369), and a second amino acid sequence being at least 70%, optionally at least 80%, preferably at least 85%, more preferably at least 90% and most preferably at least 95% homologous to a polypeptide having the sequence VALLPAHCLGWERGDCFWKGPSPFCAQVSFPRR (SEQ ID NO:434) corresponding to amino acids 296-328 of S56200_P24 (SEQ ID NO:369), wherein said first amino acid sequence and second amino acid sequence are contiguous and in a sequential order.

B. An isolated polypeptide encoding for an edge portion of S56200_P24 (SEQ ID NO:369), comprising an amino acid sequence being at least 70%, optionally at least about 80%, preferably at least about 85%, more preferably at least about 90% and most preferably at least about 95% homologous to the sequence VALLPAHCLGWERGDCFWKGPSPFCAQVSFPRR (SEQ ID NO:434) of S56200_P24 (SEQ ID NO:369).

3. Comparison report between S56200_P24 (SEQ ID NO:369) and P05164-3 (SEQ ID NO:364):

A. An isolated chimeric polypeptide encoding for S56200_P24 (SEQ ID NO:369), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRESIKQRLRSGSASPMELLSYFKQPVAATRTAVRAADY LHVALDLLERKLRSLWRRPFNVTDVLTPAQLNVLSKSSGCAYQDVGVTCPEQDKYRTIT GMCNNR corresponding to amino acids 1-183 of P05164-3 (SEQ ID NO:364), which also corresponds to amino acids 1-183 of S56200_P24 (SEQ ID NO:369), a second amino acid sequence being at least 90% homologous to RSPTLGASNRAFVRWLPAEYEDGFSLPYGWTPGVKRNGFPVALARAVSNEIVRFPTDQL TPDQERSLMFMQWGQLLDHDLDFTPEPAARASFVTGVNCETSCVQQPPCFPLK corresponding to amino acids 216-327 of P05164-3 (SEQ ID NO:364), which also corresponds to amino acids 184-295 of S56200_P24 (SEQ ID NO:369), and a third amino acid sequence being at least 70%, optionally at least 80%, preferably at least 85%, more preferably at least 90% and most preferably at least 95% homologous to a polypeptide having the sequence VALLPAHCLGWERGDCFWKGPSPFCAQVSFPRR (SEQ ID NO:434) corresponding to amino acids 296-328 of S56200_P24 (SEQ ID NO:369), wherein said first amino acid sequence, second amino acid sequence and third amino acid sequence are contiguous and in a sequential order.

B. An isolated chimeric polypeptide encoding for an edge portion of S56200_P24 (SEQ ID NO:369), comprising a polypeptide having a length “n”, wherein n is at least about 10 amino acids in length, optionally at least about 20 amino acids in length, preferably at least about 30 amino acids in length, more preferably at least about 40 amino acids in length and most preferably at least about 50 amino acids in length, wherein at least two amino acids comprise RR, having a structure as follows: a sequence starting from any of amino acid numbers 183-x to 183; and ending at any of amino acid numbers 184+((n−2)−x), in which x varies from 0 to n−2.

C. An isolated polypeptide encoding for an edge portion of S56200_P24 (SEQ ID NO:369), comprising an amino acid sequence being at least 70%, optionally at least about 80%, preferably at least about 85%, more preferably at least about 90% and most preferably at least about 95% homologous to the sequence VALLPAHCLGWERGDCFWKGPSPFCAQVSFPRR (SEQ ID NO:434) of S56200_P24 (SEQ ID NO:369).

4. Comparison report between S56200_P24 (SEQ ID NO:369) and P05164-2 (SEQ ID NO:362):

A. An isolated chimeric polypeptide encoding for S56200_P24 (SEQ ID NO:369), comprising a first amino acid sequence being at least 70%, optionally at least 80%, preferably at least 85%, more preferably at least 90% and most preferably at least 95%, homologous to a polypeptide having the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) corresponding to amino acids 1-95 of S56200_P24 (SEQ ID NO:369), a second amino acid sequence being at least 90% homologous to MELLSYFKQPVAATRTAVRAADYLHVALDLLERKLRSLWRRPFNVTDVLTPAQLNVLS KSSGCAYQDVGVTCPEQDKYRTITGMCNNRRSPTLGASNRAFVRWLPAEYEDGFSLPYG WTPGVKRNGFPVALARAVSNEIVRFPTDQLTPDQERSLMFMQWGQLLDHDLDFTPEPA ARASFVTGVNCETSCVQQPPCFPLK corresponding to amino acids 1-200 of P05164-2 (SEQ ID NO:362), which also corresponds to amino acids 96-295 of S56200_P24 (SEQ ID NO:369), and a third amino acid sequence being at least 70%, optionally at least 80%, preferably at least 85%, more preferably at least 90% and most preferably at least 95% homologous to a polypeptide having the sequence VALLPAHCLGWERGDCFWKGPSPFCAQVSFPRR (SEQ ID NO:434) corresponding to amino acids 296-328 of S56200_P24 (SEQ ID NO:369), wherein said first amino acid sequence, second amino acid sequence and third amino acid sequence are contiguous and in a sequential order.

B. An isolated polypeptide encoding for a head of S56200_P24 (SEQ ID NO:369), comprising a polypeptide being at least 70%, optionally at least about 80%, preferably at least about 85%, more preferably at least about 90% and most preferably at least about 95% homologous to the sequence MGVPFFSSLRCMVDLGPCWAGGLTAEMKLLLALAGLLAILATPQPSEGAAPAVLGEVDT SLVLSSMEEAKQLVDKAYKERRESIKQRLRSGSASP (SEQ ID NO:427) of S56200_P24 (SEQ ID NO:369).

C. An isolated polypeptide encoding for an edge portion of S56200_P24 (SEQ ID NO:369), comprising an amino acid sequence being at least 70%, optionally at least about 80%, preferably at least about 85%, more preferably at least about 90% and most preferably at least about 95% homologous to the sequence VALLPAHCLGWERGDCFWKGPSPFCAQVSFPRR (SEQ ID NO:434) of S56200_P24 (SEQ ID NO:369).

The localization of the variant protein was determined according to results from a number of different software programs and analyses, including analyses from SignalP and other specialized programs. The variant protein is believed to be secreted.

Variant protein S56200_P24 (SEQ ID NO:369) also has the following non-silent SNPs (Single Nucleotide Polymorphisms) as listed in Table 206, (given according to their position(s) on the amino acid sequence, with the alternative amino acid(s) listed).

TABLE 206
Amino acid mutations

| SNP position(s) on amino acid | Alternative | |
|---|---|---|
| sequence | amino acid(s) | Previously known SNP? |
| 36 | L -> P | Yes |
| 36 | L -> R | Yes |
| 53 | V -> F | Yes |

The glycosylation sites of variant protein S56200_P24 (SEQ ID NO:369), as compared to the known protein Myeloperoxidase precursor (SEQ ID NO:361), are described in Table 207 (given according to their position(s) on the amino acid sequence in the first column; the second column indicates whether the glycosylation site is present in the variant protein; and the last column indicates whether the position is different on the variant protein).

TABLE 207
Glycosylation site(s)

| Position(s) on known amino | | Position(s) on |
|---|---|---|
| acid sequence | Present in variant protein? | variant protein |
| 355 | No | |
| 391 | No | |
| 483 | No | |

The phosphorylation sites of variant protein S56200_P24 (SEQ ID NO:369), as compared to the known protein, are described in Table 208 (given according to their position(s) on the amino acid sequence in the first column; the second column indicates whether the phosphorylation site is present in the variant protein; and the last column indicates whether the position is different on the variant protein).

TABLE 208
Phosphorylation site(s)

| Position(s) on known | Present in | Position(s) on |
|---|---|---|
| amino acid sequence | variant protein? | variant protein |
| 316 | No | |

The variant protein has the following domains, as determined by using InterPro. The domains are described in Table 209:

TABLE 209
InterPro domain(s)

| Domain description | Analysis type | Position(s) on protein |
|---|---|---|
| Animal haem peroxidase | FPrintScan | 197-208, 251-266 |
| Animal haem peroxidase | ProfileScan | 164-328 |

Variant protein S56200_P24 (SEQ ID NO:369) is encoded by the transcript S56200_T5 (SEQ ID NO:330), for which the coding portion starts at position 196 and ends at position 1179. The transcript also has the following SNPs as listed in Table 210 (given according to their position on the nucleotide sequence, with the alternative nucleic acid listed).

TABLE 210
Nucleic acid SNPs

| SNP position(s) on | Alternative | Previously |
|---|---|---|
| nucleotide sequence | nucleic acid(s) | known SNP? |
| 171 | G -> A | Yes |
| 300 | G -> C | Yes |
| 302 | T -> G | Yes |
| 302 | T -> C | Yes |
| 352 | G -> T | Yes |
| 1992 | T -> C | No |
| 2046 | T -> C | No |
| 2470 | C -> T | No |
| 2597 | A -> C | No |
| 2597 | A -> T | No |
| 2719 | T -> C | No |
| 2831 | A -> G | No |
| 3137 | C -> | No |
| 3206 | A -> G | Yes |
| 3406 | A -> C | No |
| 3448 | C -> T | Yes |
| 3856 | T -> A | Yes |

Variant protein S56200_P31 (SEQ ID NO:370) according to the present invention is encoded by transcript S56200_T10 (SEQ ID NO:333) One or more alignments to one or more previously published protein sequences are given in the alignment table on the attached CD-ROM. A brief description of the relationship of the variant protein according to the present invention to each such aligned protein is as follows:

1. Comparison report between S56200_P31 (SEQ ID NO:370) and PERM_HUMAN (SEQ ID NO:371):

A. An isolated chimeric polypeptide encoding for S56200_P31 (SEQ ID NO:370), comprising a first amino acid sequence being at least 90% homologous to MGVPFFSSLRCMVDLGPCWAGGLTAEMKLLLALAGLLAILATPQPSEGAAPAVLGEVDT SLVLSSMEEAKQLVDKAYKERRE corresponding to amino acids 1-82 of PERM_HUMAN (SEQ ID NO:371), which also corresponds to amino acids 1-82 of S56200_P31 (SEQ ID NO:370), and a second amino acid sequence being at least 70%, optionally at least 80%, preferably at least 85%, more preferably at least 90% and most preferably at least 95% homologous to a polypeptide having the sequence RWGTRHCPALGKDVPGLSEKQQAAGSLKHQAAASQRLSQPHGTPILLQAAGGSHQDGG EGR (SEQ ID NO:437) corresponding to amino acids 83-143 of S56200_P31 (SEQ ID NO:370), wherein said first amino acid sequence and second amino acid sequence are contiguous and in a sequential order.

B. An isolated polypeptide encoding for an edge portion of S56200_P31 (SEQ ID NO:370), comprising an amino acid sequence being at least 70%, optionally at least about 80%, preferably at least about 85%, more preferably at least about 90% and most preferably at least about 95% homologous to the sequence RWGTRHCPALGKDVPGLSEKQQAAGSLKHQAAASQRLSQPHGTPILLQAAGGSHQDGG EGR (SEQ ID NO:437) of S56200_P31 (SEQ ID NO:370).

The localization of the variant protein was determined according to results from a number of different software programs and analyses, including analyses from SignalP and other specialized programs. The variant protein is believed to be secreted.

Variant protein S56200_P31 (SEQ ID NO:370) also has the following non-silent SNPs (Single Nucleotide Polymorphisms) as listed in Table 211, (given according to their position(s) on the amino acid sequence, with the alternative amino acid(s) listed).

TABLE 211
Amino acid mutations

| SNP position(s) on amino acid | Alternative |
|---|---|
| sequence | amino acid(s) |
| 36 | L -> P |
| 36 | L -> R |
| 53 | V -> F |
| 101 | E -> Q |

The glycosylation sites of variant protein S56200_P31 (SEQ ID NO:370), as compared to the known protein Myeloperoxidase precursor (SEQ ID NO:361), are described in Table 212 (given according to their position(s) on the amino acid sequence in the first column; the second column indicates whether the glycosylation site is present in the variant protein; and the last column indicates whether the position is different on the variant protein).

TABLE 212
Glycosylation site(s)

| Position(s) on known amino | |
|---|---|
| acid sequence | Present in variant protein? |
| 355 | No |
| 391 | No |
| 483 | No |

The phosphorylation sites of variant protein S56200_P31 (SEQ ID NO:370), as compared to the known protein, are described in Table 213 (given according to their position(s) on the amino acid sequence in the first column; the second column indicates whether the phosphorylation site is present in the variant protein; and the last column indicates whether the position is different on the variant protein).

TABLE 213
Phosphorylation site(s)

| Position(s) on known amino | |
|---|---|
| acid sequence | Present in variant protein? |
| 316 | No |

Variant protein S56200_P31 (SEQ ID NO:370) is encoded by the transcript S56200_T10 (SEQ ID NO:333), for which the coding portion starts at position 196 and ends at position 624. The transcript also has the following SNPs as listed in Table 214 (given according to their position on the nucleotide sequence, with the alternative nucleic acid listed).

TABLE 214
Nucleic acid SNPs

| SNP position(s) on nucleotide | Alternative |
|---|---|
| sequence | nucleic acid(s) |
| 171 | G -> A |
| 300 | G -> C |
| 302 | T -> G |
| 302 | T -> C |
| 352 | G -> T |
| 496 | G -> C |
| 1212 | T -> C |
| 1266 | T -> C |
| 1690 | C -> T |
| 1817 | A -> C |
| 1817 | A -> T |
| 1939 | T -> C |
| 2051 | A -> G |
| 2357 | C -> |
| 2426 | A -> G |
| 2626 | A -> C |
| 2668 | C -> T |
| 3076 | T -> A |

As noted above, cluster S56200 features 26 segment(s), which were listed in Table 182 above and for which the sequence(s) are given at the end of the application. These segment(s) are portions of nucleic acid sequence(s) which are described herein separately because they are of particular interest. A description of some of these segments according to the present invention is now provided.

Segment cluster S56200_N18 (SEQ ID NO:341) according to the present invention is supported by 11 libraries. The number of libraries was determined as previously described. This segment can be found in the following transcript(s): S56200_T5 (SEQ ID NO:330). Table 215 below describes the starting and ending position of this segment on each transcript.

TABLE 215
Segment location on transcripts

| | Segment | Segment |
|---|---|---|
| Transcript name | starting position | ending position |
| S56200_T5 (SEQ ID NO: 330) | 1081 | 1942 |

Segment cluster S56200_N19 (SEQ ID NO:342) according to the present invention is supported by 51 libraries. The number of libraries was determined as previously described. This segment can be found in the following transcript(s): S56200_T10 (SEQ ID NO:333), S56200_T11 (SEQ ID NO:334), S56200_T3 (SEQ ID NO:329), S56200_T5 (SEQ ID NO:330), S56200_T7 (SEQ ID NO:331) and S56200_T8 (SEQ ID NO:332). Table 216 below describes the starting and ending position of this segment on each transcript.

TABLE 216
Segment location on transcripts

| | Segment | Segment |
|---|---|---|
| Transcript name | starting position | ending position |
| S56200_T10 (SEQ ID NO: 333) | 1163 | 1481 |
| S56200_T11 (SEQ ID NO: 334) | 1081 | 1399 |
| S56200_T3 (SEQ ID NO: 329) | 1081 | 1399 |
| S56200_T5 (SEQ ID NO: 330) | 1943 | 2261 |
| S56200_T7 (SEQ ID NO: 331) | 1081 | 1399 |
| S56200_T8 (SEQ ID NO: 332) | 1081 | 1399 |

Segment cluster S56200_N33 (SEQ ID NO:344) according to the present invention is supported by 6 libraries. The number of libraries was determined as previously described. This segment can be found in the following transcript(s): S56200_T8 (SEQ ID NO:332). Table 217 below describes the starting and ending position of this segment on each transcript.

TABLE 217
Segment location on transcripts

| | Segment | Segment |
|---|---|---|
| Transcript name | starting position | ending position |
| S56200_T8 (SEQ ID NO: 332) | 1817 | 2311 |

Segment cluster S56200_N38 (SEQ ID NO:345) according to the present invention is supported by 45 libraries. The number of libraries was determined as previously described. This segment can be found in the following transcript(s): S56200_T10 (SEQ ID NO:333), S56200_T11 (SEQ ID NO:334), S56200_T3 (SEQ ID NO:329), S56200_T5 (SEQ ID NO:330), S56200_T7 (SEQ ID NO:331) and S56200_T8 (SEQ ID NO:332). Table 218 below describes the starting and ending position of this segment on each transcript.

TABLE 218
Segment location on transcripts

| | Segment | Segment |
|---|---|---|
| Transcript name | starting position | ending position |
| S56200_T10 (SEQ ID NO: 333) | 2070 | 2273 |
| S56200_T11 (SEQ ID NO: 334) | 1827 | 2030 |
| S56200_T3 (SEQ ID NO: 329) | 2046 | 2249 |
| S56200_T5 (SEQ ID NO: 330) | 2850 | 3053 |
| S56200_T7 (SEQ ID NO: 331) | 1988 | 2191 |
| S56200_T8 (SEQ ID NO: 332) | 2483 | 2686 |

According to an optional embodiment of the present invention, short segments related to the above cluster are also provided. These segments are up to about 120 bp in length, and so are included in a separate description.

Segment cluster S56200_N5 (SEQ ID NO:351) according to the present invention is supported by 1 libraries. The number of libraries was determined as previously described. This segment can be found in the following transcript(s): S56200_T10 (SEQ ID NO:333). Table 219 below describes the starting and ending position of this segment on each transcript.

TABLE 219
Segment location on transcripts

| | Segment | Segment |
|---|---|---|
| Transcript name | starting position | ending position |
| S56200_T10 (SEQ ID NO: 333) | 444 | 525 |

Segment cluster S56200_N20 (SEQ ID NO:353) according to the present invention is supported by 2 libraries. The number of libraries was determined as previously described. This segment can be found in the following transcript(s): S56200_T3 (SEQ ID NO:329). Table 220 below describes the starting and ending position of this segment on each transcript.

TABLE 220
Segment location on transcripts

| | Segment | Segment |
|---|---|---|
| Transcript name | starting position | ending position |
| S56200_T3 (SEQ ID NO: 329) | 1400 | 1457 |

Segment cluster S56200_N31 (SEQ ID NO:356) according to the present invention is supported by 41 libraries. The number of libraries was determined as previously described. This segment can be found in the following transcript(s): S56200_T10 (SEQ ID NO:333), S56200_T11 (SEQ ID NO:334), S56200_T3 (SEQ ID NO:329), S56200_T5 (SEQ ID NO:330), S56200_T7 (SEQ ID NO:331) and S56200_T8 (SEQ ID NO:332). Table 221 below describes the starting and ending position of this segment on each transcript.

TABLE 221
Segment location on transcripts

| | Segment | Segment |
|---|---|---|
| Transcript name | starting position | ending position |
| S56200_T10 (SEQ ID NO: 333) | 1643 | 1672 |
| S56200_T11 (SEQ ID NO: 334) | 1400 | 1429 |
| S56200_T3 (SEQ ID NO: 329) | 1619 | 1648 |
| S56200_T5 (SEQ ID NO: 330) | 2423 | 2452 |
| S56200_T7 (SEQ ID NO: 331) | 1561 | 1590 |
| S56200_T8 (SEQ ID NO: 332) | 1561 | 1590 |

Segment cluster S56200_N41 (SEQ ID NO:360) according to the present invention is supported by 42 libraries. The number of libraries was determined as previously described. This segment can be found in the following transcript(s): S56200_T10 (SEQ ID NO:333), S56200_T11 (SEQ ID NO:334), S56200_T3 (SEQ ID NO:329), S56200_T5 (SEQ ID NO:330), S56200_T7 (SEQ ID NO:331) and S56200_T8 (SEQ ID NO:332). Table 222 below describes the starting and ending position of this segment on each transcript.

TABLE 222
Segment location on transcripts

| | Segment | Segment |
|---|---|---|
| Transcript name | starting position | ending position |
| S56200_T10 (SEQ ID NO: 333) | 2308 | 2365 |
| S56200_T11 (SEQ ID NO: 334) | 2065 | 2122 |
| S56200_T3 (SEQ ID NO: 329) | 2284 | 2341 |
| S56200_T5 (SEQ ID NO: 330) | 3088 | 3145 |
| S56200_T7 (SEQ ID NO: 331) | 2192 | 2249 |
| S56200_T8 (SEQ ID NO: 332) | 2721 | 2778 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-94. (canceled)

95. An isolated polynucleotide comprising a nucleic acid sequence as set forth in a member selected from the group consisting of SEQ ID NOs:105-119 and homologues and fragments thereof.

96. The polynucleotide of claim 95, wherein the nucleic acid sequence encodes a polypeptide having an amino acid sequence as set forth in a member selected from the group consisting of SEQ ID NOs:144-155.

97. The polynucleotide of claim 95, wherein the nucleic acid sequence encodes a polypeptide comprising contiguous amino acids having at least about 70%, 80%, 85%, 90%, 95% or 100% homology to any one of SEQ ID NO:377-386.

98. An isolated polynucleotide, comprising a nucleic acid sequence complementary to any one of the nucleic acid sequences of claim 95.

99. An isolated polynucleotide, comprising a nucleic acid sequence that hybridizes under stringent conditions to any one of the nucleic acid sequences of claim 95.

100. An isolated polypeptide having an amino acid sequence encoded by any one of the nucleic acid sequence of claim 95.

101. An isolated polypeptide comprising an amino acid sequence at least about 70%, 80%, 85%, 90% or 95% homologous to SEQ ID NOs:144-155.

102. The isolated polypeptide of claim 101, having an amino acid sequence as set forth in any one of SEQ ID NOs:144-155.

103. The polypeptide of claim 101 comprising a first portion having an amino acid sequence being at least about 90% homologous to amino acids 1-113 of SEQ ID NO:144, and a second portion having amino acid sequence being at least about 70%, 80%, 85%, 90%, 95% or 100% homologous to SEQ ID NO:377, wherein the first amino acid sequence and the second amino acid sequence are contiguous and in a sequential order.

104. The polypeptide of claim 101 comprising a first portion having an amino acid sequence being at least about 90% homologous to amino acids 1-195 of SEQ ID NO:145, and a second portion having amino acid sequence being at least about 70%, 80%, 85%, 90%, 95% or 100% homologous to SEQ ID NO:378, wherein the first amino acid sequence and the second amino acid sequence are contiguous and in a sequential order.

105. The polypeptide of claim 101 comprising a first portion having an amino acid sequence being at least about 90% homologous to amino acids 1-212 of SEQ ID NO:146, and a second portion having amino acid sequence being at least about 70%, 80%, 85%, 90%, 95% or 100% homologous to SEQ ID NO:379, wherein the first amino acid sequence and the second amino acid sequence are contiguous and in a sequential order.

106. The polypeptide of claim 101 comprising a first portion having an amino acid sequence being at least about 90% homologous to amino acids 1-172 of SEQ ID NO:147, and a second portion having amino acid sequence being at least about 70%, 80%, 85%, 90%, 95% or 100% homologous to SEQ ID NO:380, wherein the first amino acid sequence and the second amino acid sequence are contiguous and in a sequential order.

107. The polypeptide of claim 101 comprising a first portion having an amino acid sequence being at least about 90% homologous to amino acids 1-249 of SEQ ID NO:148, and a second portion having amino acid sequence being at least about 70%, 80%, 85%, 90%, 95% or 100% homologous to SEQ ID NO:381, wherein the first amino acid sequence and the second amino acid sequence are contiguous and in a sequential order.

108. The polypeptide of claim 101 comprising a first portion having an amino acid sequence being at least about 90% homologous to amino acids 1-245 of SEQ ID NO:149, and a second portion having amino acid sequence being at least about 70%, 80%, 85%, 90%, 95% or 100% homologous to SEQ ID NO:382, wherein the first amino acid sequence and the second amino acid sequence are contiguous and in a sequential order.

109. The polypeptide of claim 101 comprising a first portion having an amino acid sequence being at least about 90% homologous to amino acids 1-136 of SEQ ID NO:150, and a second portion having amino acid sequence being at least 90% homologous to amino acids 137-270 of SEQ ID NO:150, wherein said first amino acid sequence and second amino acid sequence are contiguous and in a sequential order.

110. The polypeptide of claim 101 comprising a first portion having an amino acid sequence being at least about 90% homologous to amino acids 1-32 of SEQ ID NO:151, and a second portion having an amino acid sequence being at least 90% homologous to amino acids 33-258 of SEQ ID NO:151, wherein said first amino acid sequence and second amino acid sequence are contiguous and in a sequential order.

111. The polypeptide of claim 101 comprising a first portion having an amino acid sequence being at least about 90% homologous to amino acids 1-113 of SEQ ID NO:152, and a second portion having amino acid sequence being at least about 70%, 80%, 85%, 90%, 95% or 100% homologous to SEQ ID NO:383, wherein the first amino acid sequence and the second amino acid sequence are contiguous and in a sequential order.

112. The polypeptide of claim 101 comprising a first portion having an amino acid sequence being at least about 90% homologous to amino acids 1-139 of SEQ ID NO:153, and a second portion having amino acid sequence being at least about 70%, 80%, 85%, 90%, 95% or 100% homologous to SEQ ID NO:384, wherein the first amino acid sequence and the second amino acid sequence are contiguous and in a sequential order.

113. The polypeptide of claim 101 comprising a first portion having an amino acid sequence being at least about 90% homologous to amino acids 1-32 of SEQ ID NO:154, and a second portion having amino acid sequence being at least about 70%, 80%, 85%, 90%, 95% or 100% homologous to SEQ ID NO:385, wherein the first amino acid sequence and the second amino acid sequence are contiguous and in a sequential order.

114. The polypeptide of claim 101 comprising a first portion having an amino acid sequence 1-4 of SEQ ID NO:155, a second portion having an amino acid sequence being at least about 90% homologous to amino acids 5-31 of SEQ ID NO:155, and a third portion having an amino acid sequence being at least about 70%, 80%, 85%, 90%, 95% or 100% homologous to SEQ ID NO:385, wherein the first amino acid sequence, the second amino acid sequence and the third amino acid sequence are contiguous and in a sequential order.

115. A polypeptide comprising an amino acid sequence at least about 70%, 80%, 85%, 90%, 95% or 100% homologous to any one of SEQ ID NO:377-386.

116. An antibody which binds to at least one epitope of a polypeptide having an amino acid sequence according to claim 101.

117. The antibody of claim 116, wherein said antibody is capable of differentiating between a polypeptide having the epitope and a corresponding known protein.

118. An expression vector comprising any one of the polynucleotide sequence according to claim 95.

119. A host cell comprising the vector according to claim 118.

120. A process for producing a polypeptide comprising: culturing the host cell according to claim 119 under conditions suitable to produce the polypeptide encoded by the polynucleotide; and recovering said polypeptide.

121. An isolated polynucleotide segment, consisting of a nucleic acid sequence selected from the group consisting of: SEQ ID NOs:120-142.

122. An isolated polynucleotide segment consisting of a nucleic acid sequence complementary to any one of the nucleic acid sequences of the segments of claim 121.

123. An isolated polynucleotide segment consisting of a nucleic acid sequence that hybridizes under stringent conditions to any one of the nucleic acid sequences of the segments of claim 121.

124. A kit for detecting renal disease and/or condition, comprising a marker capable of detecting in a sample differential expression of at least one AA703666 protein or a variant thereof or a polynucleotide encoding same selected from the group consisting of:
a polypeptide comprising an amino acid sequence at least about 70%, 80%, 85%, 90%, 95% or 100% homologous to any one of SEQ ID NOs:144-155; and
a polypeptide comprising an amino acid sequence at least about 70%, 80%, 85%, 90%, 95% or 100% homologous to any on of SEQ ID NO:377-386; and
a polynucleotide comprising a nucleic acid sequence set forth in a member selected from the group consisting of SEQ ID NOs:105-119 and homologues and fragments thereof; and
a polynucleotide comprising a nucleic acid sequence set forth in a member selected from the group consisting of SEQ ID NOs:120-142, 158, 161, 164.

125. The kit of claim 124, wherein said kit comprises an antibody which binds to at least one epitope of a polypeptide having an amino acid sequence at least about 70%, 80%, 85%, 90% or 95% homologous to SEQ ID NOs:144-155, and wherein said kit further comprises at least one reagent for performing an immunoassay.

126. The kit of claim 125, wherein said immunoassay is selected from the group consisting of an ELISA, a RIA (radio immunoassay), a slot blot, immunohistochemical assay, FACS (fluorescence activated cell sorting), a radio-imaging assay or a Western blot.

127. The kit of claim 124, wherein said kit comprises at least one oligonucleotide, probe or primer pair.

128. The kit of claim 127, wherein said kit comprises at least one oligonucleotide capable of selectively hybridizing to a nucleic acid sequence as set forth in any one of SEQ ID NOs:105-142, or a homologue or fragment thereof.

129. The kit of claim 127, wherein said at least one primer pair amplifies an amplicon comprising the sequence as set forth in any one of SEQ ID NOs:158, 161, 164.

130. The primer pair of claim 129, comprising a pair of isolated oligonucleotides selected from the group consisting of SEQ ID NO:156 and 157; SEQ ID NO:159 and 160; SEQ ID NO:162 and 163.

131. A method for at least one of detecting renal disease and/or condition, monitoring renal disease and/or condition progression, monitoring renal disease and/or condition-treatment efficacy, detecting acute or chronic exacerbation of renal disease and/or condition and selecting a therapy for renal disease and/or condition, comprising detecting in a sample differential expression of at least one polypeptide of a AA703666 protein or a variant thereof selected from the group consisting of:
a polypeptide comprising an amino acid sequence at least about 70%, 80%, 85%, 90%, 95% or 100% homologous to any one of SEQ ID NOs:144-155; and a polypeptide comprising an amino acid sequence at least about 70%, 80%, 85%, 90%, 95% or 100% homologous to any on of SEQ ID NO:377-386.

132. The method of claim 131, wherein detecting the differential expression of the polypeptide is performed with an antibody which binds to at least one epitope of a polypeptide having an amino acid sequence at least about 70%, 80%, 85%, 90% or 95% homologous to SEQ ID NOs:144-155.

133. A method for at least one of detecting renal disease and/or condition, monitoring renal disease and/or condition progression, monitoring renal disease and/or condition-treatment efficacy, detecting acute or chronic exacerbation of renal disease and/or condition and selecting a therapy for renal disease and/or condition, comprising detecting in a sample differential expression of at least one polynucleotide or a part thereof, encoding a AA703666 protein or a variant thereof, selected from the group consisting of:
a polynucleotide comprising a nucleic acid sequence set forth in a member selected from the group consisting of SEQ ID NOs:105-119 and homologues and fragments thereof; and polynucleotide comprising a nucleic acid sequence set forth in a member selected from the group consisting of SEQ ID NOs: 120-142, 158, 161, 164.

134. The method of claim 133, wherein detecting the differential expression of the at least one polynucleotide is performed using a pair of isolated oligonucleotides selected from the group consisting of SEQ ID NO:156 and 157; SEQ ID NO:159 and 160; SEQ ID NO:162 and 163.
